(19) United States
(12) Patent Application Publication
(10) Pub. No.: US 2023/0203202 A1
(43) Pub. Date: Jun. 29, 2023

(54) PROTEINS BINDING NKG2D, CD16 AND 5T4

(71) Applicant: Dragonfly Therapeutics, Inc., Waltham, MA (US)

(72) Inventors: Mitchell Bigelow, Cambridge, MA (US); Mark DeRose, Wilmington, MA (US); Jinyan Du, Waltham, MA (US); Robin Friedman, Medford, MA (US); Pyae P. Hein, Malden, MA (US); Stuart William Hicks, North Andover, MA (US); Zong Sean Juo, Taipei (TW); Xinbi Li, Waltham, MA (US); Christopher Ryan Morgan, Southborough, MA (US)

(21) Appl. No.: 18/062,453

(22) Filed: Dec. 6, 2022

Related U.S. Application Data
(60) Provisional application No. 63/375,826, filed on Sep. 15, 2022.
(60) Provisional application No. 63/287,524, filed on Dec. 8, 2021.

(51) Int. Cl. C07K 16/46; A61P 35/04

(52) U.S. Cl. CPC C07K 16/468; A61P 35/04; C07K 2317/565; C07K 2317/55; C07K 2317/622; C07K 2317/53; C07K 2317/31

(57) ABSTRACT

Multispecific binding proteins that bind NKG2D receptor, CD16, and 5T4 are described, as well as pharmaceutical compositions, formulations, and therapeutic methods of the multispecific binding proteins useful for the treatment of cancer.

### CROSS-REFERENCE TO RELATED APPLICATIONS

This application claims the benefit under 35 U.S.C. § 119(e) of U.S. Provisional Application No. 63/287,524, filed on Dec. 8, 2021, and U.S. Provisional Application No. 63/375,826, filed on Sep. 15, 2022, which are hereby incorporated herein by reference in their entireties for all purposes.

### FIELD OF THE INVENTION

The present application relates to multispecific binding proteins that bind to NKG2D, CD16, and 5T4 on a cell, pharmaceutical compositions comprising such proteins, and therapeutic methods using such proteins and pharmaceutical compositions, including for the treatment of cancer.

### SEQUENCE LISTING

The instant application contains a Sequence Listing which has been submitted electronically in .XML file format and is hereby incorporated by reference in its entirety. Said .XML copy, created on created on Nov. 22, 2022, is named DFY-124-US—NP_SL.xml and is 572,081 bytes in size.

### BACKGROUND

Despite substantial research efforts, cancer continues to be a significant clinical and financial burden in countries across the globe. According to the World Health Organization (WHO), it is the second leading cause of death. Surgery, radiation therapy, chemotherapy, biological therapy, immunotherapy, hormone therapy, stem-cell transplantation, and precision medicine are among the existing treatment modalities. Despite extensive research in these areas, a highly effective, curative solution, particularly for the most aggressive cancers, has yet to be identified. Furthermore, many of the existing anti-cancer treatment modalities have substantial adverse side effects.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Slower replicating, stem-like cells of the tumor (i.e., cancer stem cells), may be causes of clinical relapse or recurrences after traditional therapies that target the rapidly proliferating cells that comprise the bulk of the tumor. Additionally, the tumor microenvironment, including cancer-associated fibroblasts (CAFs), often promotes malignancy and inhibits cancer therapies.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e., via cytolytic granules that contain perforin and granzymes as well as via death receptor pathways. Activated NK cells also secrete inflammatory cytokines such as IFN-7 and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD16 receptors on their surface. The overall sensitivity of NK cells to activation depends on the sum of stimulatory and inhibitory signals. NKG2D is a type-II transmembrane protein that is expressed by essentially all natural killer cells where NKG2D serves as an activating receptor. NKG2D is also be found on T cells where it acts as a costimulatory receptor. The ability to modulate NK cell function via NKG2D is useful in various therapeutic contexts including malignancy.

The human trophoblast glycoprotein 5T4 is an N-glycosylated transmembrane protein. Its expression is mechanistically associated with the directional movement of cells through epithelial mesenchymal transition, facilitation of CXCL12/CXCR4 chemotaxis, and blocking of canonical Wnt/beta-catenin while favoring non-canonical pathway signaling. These processes are highly regulated in development and in adult tissues, but they help drive the spread of cancer cells. It has been shown that 5T4 has very limited expression in normal adult tissue, but is widespread in many cancers including colorectal cancer, ovarian cancer, non-small cell lung cancer, renal cancer, breast cancer, endometrial cancer, squamous cell carcinoma, head and neck squamous cell carcinoma, uterine cancer, pancreatic cancer, mesothelioma, and gastric cancer. Additionally, 5T4 has been linked to cancer stem cells (Harper J et al. Mol Cancer Ther. 2017). 5T4 may also be associated with the tumor microenvironment.

Therefore, there remains a need in the field for new and useful proteins that bind 5T4 for use in the treatment of cancer.

### SUMMARY

Accordingly, in one aspect, the present disclosure provides a protein comprising: (a) a first antigen-binding site comprising a heavy chain variable domain (VH) and a light chain variable domain (VL) of an anti-NKG2D antibody; (b) a second antigen-binding site comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 138, 139, and 140, respectively, and the VL comprises CDR1, CDR2, and CDR3 sequences comprising the amino acid sequences of SEQ ID NOs: 141, 142, and 143, respectively; and (c) an antibody Fc domain or a portion thereof sufficient to bind CD16. In another aspect, provided is a protein comprising: (a) a first antigen-binding site comprising a heavy chain variable domain (VH) and a light chain variable domain (VL) of an anti-NKG2D antibody; (b) a second antigen-binding site comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 472, 474, and 140, respectively, and the VL comprises CDR1, CDR2, and CDR3 sequences comprising the amino acid sequences of SEQ ID NOs: 141, 142, and 143, respectively, wherein the CDRs are according to Kabat numbering scheme; and (c) an antibody Fc domain or a portion thereof sufficient to bind CD16. In another aspect, provided is a protein comprising: (a) a first antigen-binding site comprising a heavy chain variable domain (VH) and a light chain variable domain (VL) of an anti-NKG2D antibody; (b) a second antigen-binding site comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 138, 482 and 483, respectively, and the VL comprises CDR1, CDR2, and CDR3 sequences comprising the amino acid sequences of SEQ ID NOs: 484, 485 and 486, respectively, wherein the CDRs are according to Chothia; and (c) an antibody Fc domain or a portion thereof sufficient to bind CD16. In another aspect, provided is a protein comprising: (a) a first antigen-binding site comprising a heavy chain variable domain (VH) and a light chain variable domain (VL) of an anti-NKG2D antibody; (b) a second antigen-binding site comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 499, 500 and 501, respectively, and the VL comprises CDR1, CDR2, and CDR3 sequences comprising the amino acid sequences of SEQ ID NOs: 502, 485 and 143, respectively, wherein the CDRs are according to IMGT; and (c) an antibody Fc domain or a portion thereof sufficient to bind CD16. In another aspect, provided is a protein comprising: (a) a first antigen-binding site comprising a heavy chain variable domain (VH) and a light chain variable domain (VL) of an anti-NKG2D antibody; (b) a second antigen-binding site comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 516, 521 and 518, respectively, and the VL comprises CDR1, CDR2, and CDR3 sequences comprising the amino acid sequences of SEQ ID NOs: 519, 522 and 486, respectively, wherein the CDRs are according to Honegger; and (c) an antibody Fc domain or a portion thereof sufficient to bind CD16. In some embodiments, the CD16 is a human CD16. In some embodiments, the human CD16 is a human CD16a (FcγRIIIa).

In some embodiments, the first antigen-binding site comprising the VH and the VL of the anti-NKG2D antibody is a Fab fragment, and the second antigen-binding site comprising the VH and the VL of the anti-5T4 antibody is an scFv. In some embodiments, the first antigen-binding site comprising the VH and the VL of the anti-NKG2D antibody is an scFv, and the second antigen-binding site comprising the VH and the VL of the anti-5T4 antibody is a Fab fragment.

In some embodiments, the protein further comprises an additional antigen-binding site comprising a VH and a VL of an anti-5T4 antibody. In some embodiments, the first antigen-binding site comprising the VH and the VL of the anti-NKG2D antibody is an scFv, and the second and the additional antigen-binding sites comprising the VH and the VL of the anti-5T4 antibody are each a Fab fragment. In some embodiments, the first antigen-binding site comprising the VH and the VL of the anti-NKG2D antibody is an scFv, and the second and the additional antigen-binding sites comprising the VH and the VL of the anti-5T4 antibody are each an scFv. In some embodiments, the amino acid sequences of the second and the additional antigen-binding sites are identical.

In some embodiments, the scFv comprising the VH and the VL of the anti-NKG2D antibody is linked to an antibody constant domain or a portion thereof sufficient to bind CD16 via a hinge comprising Ala-Ser or Gly-Ser. In some embodiments, the scFv comprising the VH and the VL of the anti-NKG2D antibody is linked to an antibody constant domain or a portion thereof sufficient to bind CD16 via a hinge comprising Ala-Ser. In some embodiments, each scFv comprising the VH and the VL of the anti-5T4 antibody is linked to an antibody constant domain or a portion thereof sufficient to bind CD16 via a hinge comprising Ala-Ser or Gly-Ser. In some embodiments, the hinge further comprises an amino acid sequence Thr-Lys-Gly.

In some embodiments, within the scFv comprising the VH and the VL of the anti-NKG2D antibody, the VH of the scFv forms a disulfide bridge with the VL of the scFv. In some embodiments, within each scFv comprising the VH and the VL of the anti-5T4 antibody, the VH of the scFv forms a disulfide bridge with the VL of the scFv. In some embodiments, the disulfide bridge is formed between C44 of the VH and C100 of the VL, numbered under the Kabat numbering scheme.

In some embodiments, within the scFv comprising the VH and the VL of the anti-NKG2D antibody, the VH is linked to the VL via a flexible linker. In some embodiments, within each scFv comprising the VH and the VL of the anti-5T4 antibody, the VH is linked to the VL via a flexible linker. In some embodiments, wherein the flexible linker comprises (G₄S)₄ (SEQ ID NO:119).

In some embodiments, within the scFv comprising the VH and the VL of the anti-NKG2D antibody, the VH is positioned at the C-terminus of the VL. In some embodiments, within each scFv comprising the VH and the VL of the anti-5T4 antibody, the VH is positioned at the C-terminus of the VL. In some embodiments, within the scFv comprising the VH and the VL of the anti-NKG2D antibody, the VH is positioned at the N-terminus of the VL. In some embodiments, within each scFv comprising the VH and the VL of the anti-5T4 antibody, the VH is positioned at the N-terminus of the VL. In some embodiments, the Fab fragment comprising the VH and the VL of the anti-NKG2D antibody is not positioned between an antigen-binding site and the Fc or the portion thereof. In some embodiments, no Fab fragment comprising the VH and the VL of the anti-5T4 antibody is positioned between an antigen-binding site and the Fc or the portion thereof. In some embodiments, the Fab fragment comprising the VH and the VL of the second antigen-binding site comprising the VH and the VL of the anti-5T4 antibody or the additional antigen-binding site comprising a VH and a VL of an anti-5T4 antibody is not positioned between an antigen-binding site and the Fc or the portion thereof.

In some embodiments, the first antigen-binding site binds human NKG2D. In some embodiments, the second antigen-binding site binds human 5T4. In some embodiments, the second antigen-binding site binds human 5T4 within an LRR1 domain.

In some embodiments, the first antigen-binding site comprising the VH and the VL of the anti-NKG2D antibody comprises a VH comprising CDR1, CDR2, and CDR3 comprising the amino acid sequences of SEQ ID NOs: 81, 82, and 112, respectively; and a VL comprising CDR1, CDR2, and CDR3 comprising the amino acid sequences of SEQ ID NOs: 86, 77, and 87, respectively.

In another aspect, the present disclosure provides a protein comprising: (a) a first antigen-binding site comprising a VH and a VL of an anti-NKG2D antibody, wherein the VH comprises CDR1, CDR2, and CDR3 sequences represented by the amino acid sequences of SEQ ID NOs: 81, 82, and 112, respectively, and the VL comprises CDR1, CDR2, and CDR3 sequences represented by the amino acid sequences of SEQ ID NOs: 86, 77, and 87; (b) a second antigen-binding site comprising a VH and a VL of an anti-5T4 antibody, wherein the VH comprises CDR1, CDR2, and CDR3 sequences represented by the amino acid sequences of SEQ ID NOs: 138, 139, and 140, respectively, and the VL comprises CDR1, CDR2, and CDR3 sequences represented by the amino acid sequences of SEQ ID NOs: 141, 142, and 143; and (c) an antibody Fc domain or a portion thereof sufficient to bind CD16. In some embodiments, the first antigen-binding site comprising the VH and the VL of the anti-NKG2D antibody comprises a VH comprising CDR1, CDR2, and CDR3 sequences represented by the amino acid sequences of SEQ ID NOs: 81, 82, and 97, respectively, and a VL comprising CDR1, CDR2, and CDR3 sequences represented by the amino acid sequences of SEQ ID NOs: 86, 77, and 87, respectively.

In another aspect, the present disclosure provides a protein comprising: (a) a first antigen-binding site comprising a VH and a VL of an anti-NKG2D antibody, wherein the VH comprises CDR1, CDR2, and CDR3 sequences represented by the amino acid sequences of SEQ ID NOs: 81, 82, and 112, respectively, and the VL comprises CDR1, CDR2, and CDR3 sequences represented by the amino acid sequences of SEQ ID NOs: 86, 77, and 87, respectively; (b) a second antigen-binding site comprising a VH and a VL of an anti-5T4 antibody, wherein the VH comprises CDR1, CDR2, and CDR3 sequences represented by the amino acid sequences of SEQ ID NOs: 472, 474, 140, respectively, and the VL comprises CDR1, CDR2, and CDR3 sequences represented by the amino acid sequences of SEQ ID NOs: 141, 142, and 143, respectively, wherein the numbering is according to Kabat numbering scheme; and (c) an antibody Fc domain or a portion thereof sufficient to bind CD16. In some embodiments, the first antigen-binding site comprising the VH and the VL of the anti-NKG2D antibody comprises a VH comprising CDR1, CDR2, and CDR3 sequences represented by the amino acid sequences of SEQ ID NOs: 81, 82, and 97, respectively; and a VL comprising CDR1, CDR2, and CDR3 sequences represented by the amino acid sequences of SEQ ID NOs: 86, 77, and 87, respectively.

In another aspect, the present disclosure provides a protein comprising: (a) a first antigen-binding site comprising a VH and a VL of an anti-NKG2D antibody, wherein the VH comprises CDR1, CDR2, and CDR3 sequences represented by the amino acid sequences of SEQ ID NOs: 381, 390 and 391, respectively, and the VL comprises CDR1, CDR2, and CDR3 sequences represented by the amino acid sequences of SEQ ID NOs: 392, 385 and 393, respectively; (b) a second antigen-binding site comprising a VH and a VL of an anti-5T4 antibody, wherein the VH comprises CDR1, CDR2, and CDR3 sequences represented by the amino acid sequences of SEQ ID NOs: 138, 482 and 483, respectively, and the VL comprises CDR1, CDR2, and CDR3 sequences represented by the amino acid sequences of SEQ ID NOs: 484, 485 and 486, respectively, wherein the numbering is according to Chothia; and (c) an antibody Fc domain or a portion thereof sufficient to bind CD16. In some embodiments, the first antigen-binding site comprising the VH and the VL of the anti-NKG2D antibody comprises a VH comprising CDR1, CDR2, and CDR3 sequences represented by the amino acid sequences of SEQ ID NOs: 381, 390 and 395, respectively; and a VL comprising CDR1, CDR2, and CDR3 sequences represented by the amino acid sequences of SEQ ID NOs: 392, 385 and 393, respectively.

In another aspect, the present disclosure provides a protein comprising: (a) a first antigen-binding site comprising a VH and a VL of an anti-NKG2D antibody, wherein the VH comprises CDR1, CDR2, and CDR3 sequences represented by the amino acid sequences of SEQ ID NOs: 422, 423 and 111, respectively, and the VL comprises CDR1, CDR2, and CDR3 sequences represented by the amino acid sequences of SEQ ID NOs: 424, 385 and 87, respectively; (b) a second antigen-binding site comprising a VH and a VL of an anti-5T4 antibody, wherein the VH comprises CDR1, CDR2, and CDR3 sequences represented by the amino acid sequences of SEQ ID NOs: 499, 500 and 501, respectively, and the VL comprises CDR1, CDR2, and CDR3 sequences represented by the amino acid sequences of SEQ ID NOs: 502, 485 and 143, respectively, wherein the numbering is according to IMGT; and (c) an antibody Fc domain or a portion thereof sufficient to bind CD16. In some embodiments, the first antigen-binding site comprising the VH and the VL of the anti-NKG2D antibody comprises a VH comprising CDR1, CDR2, and CDR3 sequences represented by the amino acid sequences of SEQ ID NOs: 422, 423 and 96, respectively; and a VL comprising CDR1, CDR2, and CDR3 sequences represented by the amino acid sequences of SEQ ID NOs: 424, 385 and 87, respectively.

In another aspect, the present disclosure provides a protein comprising: (a) a first antigen-binding site comprising a VH and a VL of an anti-NKG2D antibody, wherein the VH comprises CDR1, CDR2, and CDR3 sequences represented by the amino acid sequences of SEQ ID NOs: 462, 463 and 464, respectively, and the VL comprises CDR1, CDR2, and CDR3 sequences represented by the amino acid sequences of SEQ ID NOs: 465, 459 and 393; (b) a second antigen-binding site comprising a VH and a VL of an anti-5T4 antibody, wherein the VH comprises CDR1, CDR2, and CDR3 sequences represented by the amino acid sequences of SEQ ID NOs: 516, 521 and 518, respectively, and the VL comprises CDR1, CDR2, and CDR3 sequences represented by the amino acid sequences of SEQ ID NOs: 519, 522 and 486, wherein the numbering is according to Honegger; and (c) an antibody Fc domain or a portion thereof sufficient to bind CD16. In some embodiments, the first antigen-binding site comprising the VH and the VL of the anti-NKG2D antibody comprises a VH comprising CDR1, CDR2, and CDR3 sequences represented by the amino acid sequences of SEQ ID NOs: 462, 463 and 467, respectively; and a VL comprising CDR1, CDR2, and CDR3 sequences represented by the amino acid sequences of SEQ ID NOs: 465, 459 and 393, respectively.

In another aspect, the present disclosure provides a protein comprising: (a) a first antigen-binding site comprising a VH and a VL of an anti-NKG2D antibody, wherein the VH comprises CDR1, CDR2, and CDR3 sequences represented by the amino acid sequences of SEQ ID NOs: 81, 82, and 97, respectively, and the VL comprises CDR1, CDR2, and CDR3 sequences represented by the amino acid sequences of SEQ ID NOs: 86, 77, and 87; (b) a second antigen-binding site comprising a VH and a VL of an anti-5T4 antibody, wherein the VH comprises CDR1, CDR2, and CDR3 sequences represented by the amino acid sequences of SEQ ID NOs: 138, 139, and 140, respectively, and the VL comprises CDR1, CDR2, and CDR3 sequences represented by the amino acid sequences of SEQ ID NOs: 141, 142, and 143; and (c) an antibody Fc domain, comprising a first antibody Fc domain polypeptide or a portion thereof sufficient to bind CD16, and a second antibody Fc domain polypeptide or a portion thereof sufficient to bind CD16; and wherein the VH or the VL of the anti-NKG2D antibody is fused to the N-terminus of the first antibody Fc domain polypeptide or portion thereof sufficient to bind CD16, and the VH or the VL of the anti-5T4 antibody is fused to the N-terminus of the second antibody Fc domain polypeptide or portion thereof sufficient to bind CD16.

In some embodiments, the first antigen-binding site comprises a VH comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:95 and a VL comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:85. In some embodiments, the first antigen-binding site comprises a VH comprising an amino acid sequence of SEQ ID NO:95 and a VL comprising an amino acid sequence of SEQ ID NO:85. In some embodiments, the first antigen-binding site comprises a VH comprising an amino acid sequence at least 95% identical to SEQ ID NO:95 and a VL comprising an amino acid sequence at least 95% identical to SEQ ID NO:85. In some embodiments, the first antigen-binding site comprises a VH comprising an amino acid sequence at least 96% identical to SEQ ID NO:95 and a VL comprising an amino acid sequence at least 96% identical to SEQ ID NO:85. In some embodiments, the first antigen-binding site comprises a VH comprising an amino acid sequence at least 97% identical to SEQ ID NO:95 and a VL comprising an amino acid sequence at least 97% identical to SEQ ID NO:85. In some embodiments, the first antigen-binding site comprises a VH comprising an amino acid sequence at least 98% identical to SEQ ID NO:95 and a VL comprising an amino acid sequence at least 98% identical to SEQ ID NO:85. In some embodiments, the first antigen-binding site comprises a VH comprising an amino acid sequence at least 99% identical to SEQ ID NO:95 and a VL comprising an amino acid sequence at least 99% identical to SEQ ID NO:85. In some embodiments, the first antigen-binding site comprises a VH consisting of the amino acid sequence of SEQ ID NO:95 and a VL consisting of the amino acid sequence of SEQ ID NO:85.

In some embodiments, the second antigen-binding site comprises a VH at least 90%, e.g., at least 91%, at least 92%, at least 93%, at least 94%, at least 95%, at least 96%, at least 97%, at least 98%, at least 99% or 100%, identical to SEQ ID NO:144 and a VL at least 90%, e.g., at least 91%, at least 92%, at least 93%, at least 94%, at least 95%, at least 96%, at least 97%, at least 98%, at least 99% or 100%, identical to SEQ ID NO:145. In some embodiments, the second antigen-binding site comprises a VH comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:263 and a VL comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:145. In some embodiments, the second antigen-binding site comprises a VH with a G44C substitution relative to SEQ ID NO:144 and a VL with a G100C substitution relative to SEQ ID NO:145. In some embodiments, the second antigen-binding site comprises a VH comprising the amino acid sequence of SEQ ID NO:144 and a VL comprising the amino acid sequence of SEQ ID NO:145, or a VH comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:146 and a VL comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:147. In some embodiments, the second antigen-binding site comprises a VH comprising the amino acid sequence of SEQ ID NO:146 and a VL comprising the amino acid sequence of SEQ ID NO:147. In some embodiments, the second antigen-binding site comprises a VH comprising the amino acid sequence of SEQ ID NO:144 and a VL comprising the amino acid sequence of SEQ ID NO:145.

In some embodiments, the second antigen-binding site comprises a VH comprising the amino acid sequence at least 90%, e.g., at least 91%, at least 92%, at least 93%, at least 94%, at least 95%, at least 96%, at least 97%, at least 98%, at least 99% or 100%, identical to SEQ ID NO:146 and a VL comprising the amino acid sequence at least 90%, e.g., at least 91%, at least 92%, at least 93%, at least 94%, at least 95%, at least 96%, at least 97%, at least 98%, at least 99% or 100%, identical to SEQ ID NO:147. In some embodiments, the second antigen-binding site comprises a VH comprising the amino acid sequence at least 95% identical to SEQ ID NO:146 and a VL comprising the amino acid sequence at least 95% identical to SEQ ID NO:147. In some embodiments, the second antigen-binding site comprises a VH comprising the amino acid sequence at least 96% identical to SEQ ID NO:146 and a VL comprising the amino acid sequence at least 96% identical to SEQ ID NO:147. In some embodiments, the second antigen-binding site comprises a VH comprising the amino acid sequence at least 97% identical to SEQ ID NO:146 and a VL comprising the amino acid sequence at least 97% identical to SEQ ID NO:147. In some embodiments, the second antigen-binding site comprises a VH comprising the amino acid sequence at least 98% identical to SEQ ID NO:146 and a VL comprising the amino acid sequence at least 98% identical to SEQ ID NO:147. In some embodiments, the second antigen-binding site comprises a VH comprising the amino acid sequence at least 99% identical to SEQ ID NO:146 and a VL comprising the amino acid sequence at least 99% identical to SEQ ID NO:147. In some embodiments, the second antigen-binding site comprises a VH comprising the amino acid sequence of SEQ ID NO:146 and a VL comprising the amino acid sequence of SEQ ID NO:147. In some embodiments, the second antigen-binding site comprises a VH consisting of the amino acid sequence of SEQ ID NO:146 and a VL consisting of the amino acid sequence of SEQ ID NO:147.

In some embodiments, the second antigen-binding site comprises a single-chain fragment variable (scFv), and wherein the scFv comprises a VH comprising the amino acid sequence of SEQ ID NO:146 and a VL comprising the amino acid sequence of SEQ ID NO:147. In some embodiments, the second antigen-binding site comprises a single-chain fragment variable (scFv), and wherein the scFv comprises an amino acid sequence at least 90%, e.g., at least 91%, at least 92%, at least 93%, at least 94%, at least 95%, at least 96%, at least 97%, at least 98%, at least 99% or 100%, identical to a sequence selected from the group consisting of SEQ ID NOs: 148 and 149. In some embodiments, the second antigen-binding site comprises an scFv and the scFv comprises an amino acid sequence at least 90%, e.g., at least 91%, at least 92%, at least 93%, at least 94%, at least 95%, at least 96%, at least 97%, at least 98%, at least 99% or 100%, identical to SEQ ID NO:148. In some embodiments, the second antigen-binding site comprises an scFv and the scFv comprises an amino acid sequence at least 95% identical to SEQ ID NO:148. In some embodiments, the second antigen-binding site comprises an scFv and the scFv comprises an amino acid sequence at least 96% identical to SEQ ID NO:148. In some embodiments, the second antigen-binding site comprises an scFv and the scFv comprises an amino acid sequence at least 97% identical to SEQ ID NO:148. In some embodiments, the second antigen-binding site comprises an scFv and the scFv comprises an amino acid sequence at least 98% identical to SEQ ID NO:148. In some embodiments, the second antigen-binding site comprises an scFv and the scFv comprises an amino acid sequence at least 99% identical to SEQ ID NO:148. In some embodiments, the second antigen-binding site comprises an scFv and the scFv comprises an amino acid sequence of SEQ ID NO:148. In some embodiments, the second antigen-binding site comprises an scFv and the scFv comprises the amino acid sequence of SEQ ID NO:148.

In some embodiments, the protein comprises an amino acid sequence at least 90% identical, e.g., at least 91%, 92%, 93%, 94%, 95%, 96%, 97%, 98% or 99% identical, to SEQ ID NO:198. In some embodiments, the protein comprises an amino acid sequence at least 95% identical to SEQ ID NO:198. In some embodiments, the protein comprises an amino acid sequence at least 96% identical to SEQ ID NO:198. In some embodiments, the protein comprises an amino acid sequence at least 97% identical to SEQ ID NO:198. In some embodiments, the protein comprises an amino acid sequence at least 98% identical to SEQ ID NO:198. the protein comprises an amino acid sequence at least 99% identical to SEQ ID NO: 198. In some embodiments, the protein comprises an amino acid sequence of SEQ ID NO: 198. In some embodiments, the protein comprises the amino acid sequence of SEQ ID NO: 198.

In some embodiments, the present disclosure provides a protein comprising: a first antigen-binding site comprising a VH and a VL of an anti-NKG2D antibody, wherein the VH comprises the amino acid sequence of SEQ ID NO:95 and a VL comprises the amino acid sequence of SEQ ID NO:85; a second antigen-binding site comprising a VH and a VL of an anti-5T4 antibody, wherein the VH comprises the amino acid sequence of SEQ ID NO:146 and a VL comprises the amino acid sequence of SEQ ID NO:147; and an antibody Fc domain, comprising a first antibody Fc domain polypeptide or a portion thereof sufficient to bind CD16, and a second antibody Fc domain polypeptide or a portion thereof sufficient to bind CD16.

In some embodiments, the present disclosure provides a protein comprising: a first antigen-binding site comprising a VH and a VL of an anti-NKG2D antibody, wherein the VH comprises the amino acid sequence of SEQ ID NO:95 and a VL comprises the amino acid sequence of SEQ ID NO:85; a second antigen-binding site comprising the amino acid sequence of SEQ ID NO:148; and an antibody Fc domain, comprising a first antibody Fc domain polypeptide or a portion thereof sufficient to bind CD16, and a second antibody Fc domain polypeptide or a portion thereof sufficient to bind CD16.

In another aspect, provided herein is a protein comprising: (a) a first antigen-binding site comprising a heavy chain variable domain (VH) and a light chain variable domain (VL) of an anti-NKG2D antibody; (b) a second antigen-binding site comprising a VH comprising a CDR1, a CDR2, and a CDR3 sequence selected from Table 2 and a VL comprising a CDR1, a CDR2, and a CDR3 sequence selected from Table 2, or a VH comprising a CDR1, a CDR2, and a CDR3 sequence selected from Table 12 and a VL comprising a CDR1, a CDR2, and a CDR3 sequence selected from Table 12; and (c) an antibody Fc domain or a portion thereof sufficient to bind CD16. In some embodiments, the protein comprises (a) a first antigen-binding site comprising a heavy chain variable domain (VH) and a light chain variable domain (VL) of an anti-NKG2D antibody; (b) a second antigen-binding site comprising a VH comprising a CDR1, a CDR2, and a CDR3 sequence selected from Table 2 and a VL comprising a CDR1, a CDR2, and a CDR3 sequence selected from Table 2, or a VH comprising a CDR1, a CDR2, and a CDR3 sequence selected from Table 12 and a VL comprising a CDR1, a CDR2, and a CDR3 sequence comprising the amino acid sequences of SEQ ID NOs: 189, 190, and 143, respectively; and (c) an antibody Fc domain or a portion thereof sufficient to bind CD16.

In some embodiments, the antibody Fc domain is a human IgG1 antibody Fc domain. In some embodiments, the antibody Fc domain or the portion thereof comprises an amino acid sequence at least 90%, e.g., at least 91%, at least 92%, at least 93%, at least 94%, at least 95%, at least 96%, at least 97%, at least 98%, at least 99% or 100%, identical to SEQ ID NO:118. In some embodiments, the antibody Fc domain or the portion thereof comprises an amino acid sequence at least 95% identical to SEQ ID NO:118. In some embodiments, the antibody Fc domain or the portion thereof comprises an amino acid sequence at least 96% identical to SEQ ID NO:118. In some embodiments, the antibody Fc domain or the portion thereof comprises an amino acid sequence at least 97% identical to SEQ ID NO:118. In some embodiments, the antibody Fc domain or the portion thereof comprises an amino acid sequence at least 98% identical to SEQ ID NO:118. In some embodiments, at least one polypeptide chain of the antibody Fc domain comprises one or more mutations, relative to SEQ ID NO:118, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system. In some embodiments, at least one polypeptide chain of the antibody Fc domain comprises one or more mutations, relative to SEQ ID NO:118, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, F405L, Y407A, Y407I, Y407V, K409F, K409W, K409D, K409R, T411D, T411E, K439D, and K439E, numbered according to the EU numbering system. In some embodiments, one polypeptide chain of the antibody heavy chain constant region comprises one or more mutations, relative to SEQ ID NO:118, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:118, at one or more positions selected from Q347, Y349, L351, S354, E356, E357, S364, T366, L368, K370, N390, K392, T394, D399, D401, F405, Y407, K409, T411, and K439, numbered according to the EU numbering system.

In some embodiments, one polypeptide chain of the antibody heavy chain constant region comprises K360E and K409W substitutions relative to SEQ ID NO:118; and the other polypeptide chain of the antibody heavy chain constant region comprises Q347R, D399V and F405T substitutions relative to SEQ ID NO:118, numbered according to the EU numbering system. In some embodiments, the VH of the anti-NKG2D antibody is fused to the N-terminus of a first antibody Fc domain polypeptide comprising K360E and K409W substitutions relative to SEQ ID NO:118, and the VH of the anti-5T4 antibody is fused to the N-terminus of a second antibody Fc domain polypeptide comprising Q347R, D399V and F405T substitutions relative to SEQ ID NO:118, numbered according to the EU numbering system. In some embodiments, the first antibody Fc domain polypeptide and the second antibody Fc domain polypeptide form a heterodimer. In some embodiments, heterodimer formation is facilitated by the K360E and K409W substitutions in the first antibody Fc domain polypeptide and the Q347R, D399V and F405T substitutions in the second antibody Fc domain polypeptide. In some embodiments, one polypeptide chain of the antibody heavy chain constant region comprises an F405L substitution relative to SEQ ID NO:118; and the other polypeptide chain of the antibody heavy chain constant region comprises a K409R substitution relative to SEQ ID NO:118, numbered according to the EU numbering system. In some embodiments, one polypeptide chain of the antibody heavy chain constant region comprises a Y349C substitution relative to SEQ ID NO:118; and the other polypeptide chain of the antibody heavy chain constant region comprises an S354C substitution relative to SEQ ID NO:118, numbered according to the EU numbering system. In some embodiments, the VH of the anti-NKG2D antibody is fused to the N-terminus of a first antibody Fc domain polypeptide comprising a Y349C substitution relative to SEQ ID NO:118, and the VH of the anti-5T4 antibody is fused to the N-terminus of a second antibody Fc domain polypeptide comprising a S354C substitution relative to SEQ ID NO:118, numbered according to the EU numbering system. In some embodiments, the first antibody Fc domain polypeptide forms a disulfide bridge with the second antibody Fc domain polypeptide. In some embodiments, the disulfide bridge is formed between the Y349C substitution in the first antibody Fc domain polypeptide and the S354C substitution in the second antibody Fc domain polypeptide, numbered according to the EU numbering system.

In one aspect, provided is a trispecific antibody comprising: (a) a human NKG2D-binding site which is a Fab fragment comprising a VH and VL, wherein the VH comprises CDR1, CDR2, and CDR3 sequences represented by the amino acid sequences of SEQ ID NOs: 81, 82, and 97, respectively, and the VL comprises CDR1, CDR2, and CDR3 sequences represented by the amino acid sequences of SEQ ID NOs: 86, 77, and 87, respectively, (b) a human 5T4-binding site which is an scFv comprising a VH and a VL, wherein the VH comprises CDR1, CDR2, and CDR3 sequences comprising the amino acid sequences of SEQ ID NOs: 138, 139, and 140, respectively, and the VL comprises CDR1, CDR2, and CDR3 sequences comprising the amino acid sequences of SEQ ID NOs: 141, 142, and 143, respectively, wherein the VH is positioned at the C-terminus of the VL, wherein the VH is linked to the VL via a flexible linker comprising (G4S)4 (SEQ ID NO:119), and wherein a disulfide bridge is formed between C44 of the VH and C100 of the VL, numbered under the Kabat numbering scheme, and (c) a human CD16a-binding site which is a human IgG1 antibody Fc domain comprising: (i) a first Fc domain polypeptide that comprises an amino acid sequence at least 95% identical to SEQ ID NO:118 and comprising Y349C, K360E, and K409W substitutions relative to SEQ ID NO:118, numbered according to the EU numbering system, and (ii) a second Fc domain polypeptide that comprises an amino acid sequence at least 95% identical to SEQ ID NO:118 and comprising Q347R, S354C, D399V and F405T substitutions relative to SEQ ID NO:118, numbered according to the EU numbering system, wherein a disulfide bridge is formed between C349 of (i) and C354 of (ii), and wherein (i) and (iii) form a heterodimer, wherein the VH of (a) is fused to the N-terminus of the first Fc domain polypeptide, and the VH of (b) is fused to the N-terminus of the second Fc domain polypeptide via a hinge comprising Ala-Ser. In some embodiments of the trispecific antibody, the VH of (a) comprises the amino acid sequence of SEQ ID NO:95, the VL of (a) comprises the amino acid sequence of SEQ ID NO:85, the VH of (b) comprises the amino acid sequence of SEQ ID NO:146, the VL of (b) comprises the amino acid sequence of SEQ ID NO:147, the first Fc domain polypeptide comprises an amino acid sequence at least 98% identical to SEQ ID NO:118, and the second Fc domain polypeptide comprises an amino acid sequence at least 98% identical to SEQ ID NO:118. In some embodiments of the trispecific antibody, (b) comprises the amino acid sequence of SEQ ID NO:148. In some embodiments, the trispecific antibody comprises: (a) a first polypeptide comprising the amino acid sequence of SEQ ID NO:198; (b) a second polypeptide comprising the amino acid sequence of SEQ ID NO:199; and (c) a third polypeptide comprising the amino acid sequence of SEQ ID NO:200.

In another aspect, the present disclosure provides a pharmaceutical formulation comprising: (a) a protein comprising: (i) a first antigen-binding site comprising a heavy chain variable domain (VH) and a light chain variable domain (VL) of an anti-NKG2D antibody; (ii) a second antigen-binding site comprising a VH and a VL of an anti-5T4 antibody; and (iii) an antibody Fc domain or a portion thereof sufficient to bind CD16, (b) a buffer comprising citrate; (c) sucrose; and (d) a polysorbate, wherein the pH of the pharmaceutical formulation is 6.0 to 7.0.

In some embodiments, the concentration of the protein in the pharmaceutical formulation is 1 mg/mL to 125 mg/mL. In some embodiments, the concentration of the protein in the pharmaceutical formulation is 2 mg/mL to 100 mg/mL. In some embodiments, the concentration of the protein in the pharmaceutical formulation is 5 mg/mL to 50 mg/mL. In some embodiments, the concentration of the protein in the pharmaceutical formulation is 7.5 mg/mL to 25 mg/mL. In some embodiments, the concentration of the protein in the pharmaceutical formulation is 10 mg/mL to 20 mg/mL. In some embodiments, the concentration of the protein in the pharmaceutical formulation is about 15 mg/mL.

In some embodiments, the concentration of citrate in the pharmaceutical formulation is 15 mM to 25 mM. In some embodiments, the concentration of citrate in the pharmaceutical formulation is 17.5 mM to 22.5 mM. In some embodiments, the concentration of citrate in the pharmaceutical formulation is about 20 mM. In some embodiments, citrate in the pharmaceutical formulation comprises sodium citrate, citric acid, or a combination thereof. In some embodiments, the buffer in the pharmaceutical formulation comprises a combination of sodium citrate and citric acid. In some embodiments, the concentration of sodium citrate in the pharmaceutical formulation is 17 mM to 21 mM. In some embodiments, the concentration of sodium citrate in the pharmaceutical formulation is about 18.9 mM. In some embodiments, the concentration of citric acid in the pharmaceutical formulation is 0.5 mM to 1.5 mM. In some embodiments, the concentration of citric acid in the pharmaceutical formulation is about 1.1 mM. In some embodiments, the pH of the buffer in the pharmaceutical formulation is 6.0 to 7.0. In some embodiments, the pH of the buffer in the pharmaceutical formulation is 6.5.

In some embodiments, the concentration of sucrose in the pharmaceutical formulation is 170 mM to 180 mM. In some embodiments, the concentration of sucrose in the pharmaceutical formulation is 172.5 mM to 177.5 mM. In some embodiments, the concentration of sucrose in the pharmaceutical formulation is about 175.2 mM.

In some embodiments, the polysorbate in the pharmaceutical formulation is polysorbate 80. In some embodiments, the concentration of the polysorbate in the pharmaceutical formulation is 0.05 mg/mL to 0.15 mg/mL. In some embodiments, the concentration of the polysorbate in the pharmaceutical formulation is about 0.1 mg/mL. In some embodiments, the pH of the pharmaceutical formulation is 6.5.

The present disclosure also provides a vial comprising a pharmaceutical formulation comprising: (a) a protein comprising: (i) a first antigen-binding site comprising a heavy chain variable domain (VH) and a light chain variable domain (VL) of an anti-NKG2D antibody; (ii) a second antigen-binding site comprising a VH and a VL of an anti-5T4 antibody; and (iii) an antibody Fc domain or a portion thereof sufficient to bind CD16; (b) a buffer comprising citrate; (c) sucrose; and (d) a polysorbate, wherein the pH of the pharmaceutical formulation is 6.0 to 7.0. In some embodiments, the vial comprises 100 mg to 200 mg of the protein. In some embodiments, the vial comprises about 150 mg of the protein.

In some embodiments, the vial comprises 50 mg to 60 mg of sodium citrate. In some embodiments, the vial comprises about 55.5 mg of sodium citrate. In some embodiments, the vial comprises 1.5 mg to 3 mg of citric acid. In some embodiments, the vial comprises about 2.3 mg of citric acid. In some embodiments, the vial comprises 500 mg to 700 mg of sucrose. In some embodiments, the vial comprises about 600 mg of sucrose. In some embodiments, the polysorbate in the pharmaceutical formulation is polysorbate 80. In some embodiments, the vial comprises 0.5 mg to 1.5 mg of polysorbate 80. In some embodiments, the vial comprises about 1 mg of polysorbate 80. In some embodiments, the pH of the pharmaceutical formulation is 6.5. In some embodiments, the vial comprises about 10 mL of the pharmaceutical formulation.

In some embodiments, more than 93% of the protein in the pharmaceutical formulation has native conformation as determined by size-exclusion chromatography, after incubation at 50° C. for 28 days.

In some embodiments, the protein in the pharmaceutical formulation comprises: (a) a first antigen-binding site comprising a heavy chain variable domain (VH) and a light chain variable domain (VL) of an anti-NKG2D antibody; (b) a second antigen-binding site comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 138, 139, and 140, respectively, and the VL comprises CDR1, CDR2, and CDR3 sequences comprising the amino acid sequences of SEQ ID NOs: 141, 142, and 143, respectively; and (c) an antibody Fc domain or a portion thereof sufficient to bind CD16.

In some embodiments, the protein in the pharmaceutical formulation comprises a first antigen-binding site wherein the VH and the VL of the anti-NKG2D antibody is a Fab fragment, and the second antigen-binding site comprising the VH and the VL of the anti-5T4 antibody is an scFv. In some embodiments, the first antigen-binding site comprising the VH and the VL of the anti-NKG2D antibody is an scFv, and the second antigen-binding site comprising the VH and the VL of the anti-5T4 antibody is a Fab fragment.

In some embodiments, the protein in the pharmaceutical formulation further comprises an additional antigen-binding site comprising a VH and a VL of an anti-5T4 antibody. In some embodiments, the first antigen-binding site that comprises the VH and the VL of the anti-NKG2D antibody is an scFv, and the second and the additional antigen-binding sites comprising the VH and the VL of the anti-5T4 antibody are each a Fab fragment. In some embodiments, the first antigen-binding site comprising the VH and the VL of the anti-NKG2D antibody is an scFv, and the second and the additional antigen-binding sites comprising the VH and the VL of the anti-5T4 antibody are each an scFv. In some embodiments, the amino acid sequences of the second and the additional antigen-binding sites are identical.

In some embodiments, the protein in the pharmaceutical formulation comprises an scFv comprising the VH and the VL of the anti-NKG2D antibody is linked to an antibody constant domain or a portion thereof sufficient to bind CD16 via a hinge comprising Ala-Ser or Gly-Ser. In some embodiments, the protein in the pharmaceutical formulation comprises an scFv comprising the VH and the VL of the anti-NKG2D antibody is linked to an antibody constant domain or a portion thereof sufficient to bind CD16 via a hinge comprising Ala-Ser. In some embodiments, each scFv comprising the VH and the VL of the anti-5T4 antibody is linked to an antibody constant domain or a portion thereof sufficient to bind CD16 via a hinge comprising Ala-Ser or Gly-Ser. In some embodiments, the hinge further comprises an amino acid sequence Thr-Lys-Gly.

In some embodiments, the protein in the pharmaceutical formulation comprises an scFv comprising the VH and the VL of the anti-NKG2D antibody, wherein the VH of the scFv forms a disulfide bridge with the VL of the scFv. In some embodiments, within each scFv that comprises the VH and the VL of the anti-5T4 antibody, the VH of the scFv forms a disulfide bridge with the VL of the scFv. In some embodiments, the disulfide bridge is formed between C44 of the VH and C100 of the VL, numbered under the Kabat numbering scheme.

In some embodiments, the protein in the pharmaceutical formulation comprises an scFv comprising the VH and the VL of the anti-NKG2D antibody, wherein the VH is linked to the VL via a flexible linker. In some embodiments, within each scFv comprising the VH and the VL of the anti-5T4 antibody, the VH is linked to the VL via a flexible linker. In some embodiments, the flexible linker comprises (G₄S)₄ (SEQ ID NO:119).

In some embodiments, the protein in the pharmaceutical formulation comprises an scFv comprising the VH and the VL of the anti-NKG2D antibody, wherein the VH is positioned at the C-terminus of the VL. In some embodiments, within each scFv comprising the VH and the VL of the anti-5T4 antibody, the VH is positioned at the C-terminus of the VL. In some embodiments, within the scFv comprising the VH and the VL of the anti-NKG2D antibody, the VH is positioned at the N-terminus of the VL. In some embodiments, within each scFv comprising the VH and the VL of the anti-5T4 antibody, the VH is positioned at the N-terminus of the VL.

In some embodiments, the protein in the pharmaceutical formulation comprises a Fab fragment comprising the VH and the VL of the anti-NKG2D antibody wherein the Fab fragment is not positioned between an antigen-binding site and the Fc or the portion thereof. In some embodiments, no Fab fragment comprising the VH and the VL of the anti-5T4 antibody is positioned between an antigen-binding site and the Fc or the portion thereof. In some embodiments, a Fab fragment comprising the VH and the VL of the second antigen-binding site comprising the VH and the VL of the anti-5T4 antibody or the additional antigen-binding site comprising a VH and a VL of an anti-5T4 antibody is not positioned between an antigen-binding site and the Fc or the portion thereof.

In some embodiments, the protein in the pharmaceutical formulation comprises a first antigen-binding site that binds human NKG2D; in some embodiments, the second antigen-binding site binds human 5T4. In some embodiments, the second antigen-binding site binds human 5T4 within an LRR1 domain.

In some embodiments, the protein in the pharmaceutical formulation comprises a first antigen-binding site comprising the VH and the VL of the anti-NKG2D antibody comprising a VH comprising CDR1, CDR2, and CDR3 comprising the amino acid sequences of SEQ ID NOs: 81, 82, and 112, respectively; and a VL comprising CDR1, CDR2, and CDR3 comprising the amino acid sequences of SEQ ID NOs: 86, 77, and 87, respectively.

In some embodiments, the protein in the pharmaceutical formulation comprises: (a) a first antigen-binding site comprising a VH and a VL of an anti-NKG2D antibody, the VH comprising CDR1, CDR2, and CDR3 sequences represented by the amino acid sequences of SEQ ID NOs: 81, 82, and 112, respectively, and the VL comprising CDR1, CDR2, and CDR3 sequences represented by the amino acid sequences of SEQ ID NOs: 86, 77, and 87, respectively; (b) a second antigen-binding site comprising a VH and a VL of an anti-5T4 antibody, the VH comprising CDR1, CDR2, and CDR3 sequences represented by the amino acid sequences of SEQ ID NOs: 138, 139, and 140, respectively, and the VL comprising CDR1, CDR2, and CDR3 sequences represented by the amino acid sequences of SEQ ID NOs: 141, 142, and 143, respectively; and (c) an antibody Fc domain or a portion thereof sufficient to bind CD16. In some embodiments, the first antigen-binding site comprising the VH and the VL of the anti-NKG2D antibody comprises a VH comprising CDR1, CDR2, and CDR3 sequences represented by the amino acid sequences of SEQ ID NOs: 81, 82, and 97, respectively, and a VL comprising CDR1, CDR2, and CDR3 sequences represented by the amino acid sequences of SEQ ID NOs: 86, 77, and 87, respectively.

In some embodiments, the protein in the pharmaceutical formulation comprises: (a) a first antigen-binding site comprising a VH and a VL of an anti-NKG2D antibody, the VH comprising CDR1, CDR2, and CDR3 sequences represented by the amino acid sequences of SEQ ID NOs: 81, 82, and 97, respectively, and the VL comprising CDR1, CDR2, and CDR3 sequences represented by the amino acid sequences of SEQ ID NOs: 86, 77, and 87, respectively; (b) a second antigen-binding site comprising a VH and a VL of an anti-5T4 antibody, the VH comprising CDR1, CDR2, and CDR3 sequences represented by the amino acid sequences of SEQ ID NOs: 138, 139, and 140, respectively, and the VL comprising CDR1, CDR2, and CDR3 sequences represented by the amino acid sequences of SEQ ID NOs: 141, 142, and 143, respectively; and (c) an antibody Fc domain, comprising a first antibody Fc domain polypeptide or a portion thereof sufficient to bind CD16, and a second antibody Fc domain polypeptide or a portion thereof sufficient to bind CD16; and the VH or the VL of the anti-NKG2D antibody is fused to the N-terminus of the first antibody Fc domain polypeptide or portion thereof sufficient to bind CD16, and the VH or the VL of the anti-5T4 antibody is fused to the N-terminus of the second antibody Fc domain polypeptide or portion thereof sufficient to bind CD16.

In some embodiments, the first antigen-binding site comprises a VH comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:95 and a VL comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:85. In some embodiments, the first antigen-binding site comprises a VH comprising an amino acid sequence of SEQ ID NO:95 and a VL comprising an amino acid sequence of SEQ ID NO:85. In some embodiments, the first antigen-binding site comprises a VH comprising an amino acid sequence at least 95% identical to SEQ ID NO:95 and a VL comprising an amino acid sequence at least 95% identical to SEQ ID NO:85. In some embodiments, the first antigen-binding site comprises a VH comprising an amino acid sequence at least 96% identical to SEQ ID NO:95 and a VL comprising an amino acid sequence at least 96% identical to SEQ ID NO:85. In some embodiments, the first antigen-binding site comprises a VH comprising an amino acid sequence at least 97% identical to SEQ ID NO:95 and a VL comprising an amino acid sequence at least 97% identical to SEQ ID NO:85. In some embodiments, the first antigen-binding site comprises a VH comprising an amino acid sequence at least 98% identical to SEQ ID NO:95 and a VL comprising an amino acid sequence at least 98% identical to SEQ ID NO:85. In some embodiments, the first antigen-binding site comprises a VH comprising an amino acid sequence at least 99% identical to SEQ ID NO:95 and a VL comprising an amino acid sequence at least 99% identical to SEQ ID NO:85. In some embodiments, the first antigen-binding site comprises a VH consisting of the amino acid sequence of SEQ ID NO:95 and a VL consisting of the amino acid sequence of SEQ ID NO:85.

In some embodiments, the second antigen-binding site comprises a VH at least 90%, e.g., at least 91%, at least 92%, at least 93%, at least 94%, at least 95%, at least 96%, at least 97%, at least 98%, at least 99% or 100%, identical to SEQ ID NO:144 and a VL at least 90%, e.g., at least 91%, at least 92%, at least 93%, at least 94%, at least 95%, at least 96%, at least 97%, at least 98%, at least 99% or 100%, identical to SEQ ID NO:145. In some embodiments, the second antigen-binding site comprises a VH comprising the amino acid sequence of SEQ ID NO:263 and a VL comprising the amino acid sequence of SEQ ID NO:145. In some embodiments, the second antigen-binding site comprises a VH with a G44C substitution relative to SEQ ID NO:144 or SEQ ID NO:263 and a VL with a G100C substitution relative to SEQ ID NO:145. In some embodiments, the second antigen-binding site comprises a VH comprising the amino acid sequence of SEQ ID NO:144 and a VL comprising the amino acid sequence of SEQ ID NO:145, or a VH comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:146 and a VL comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:147. In some embodiments, the second antigen-binding site comprises a VH comprising the amino acid sequence at least 95% identical to SEQ ID NO:146 and a VL comprising the amino acid sequence at least 95% identical to SEQ ID NO:147. In some embodiments, the second antigen-binding site comprises a VH comprising the amino acid sequence at least 96% identical to SEQ ID NO:146 and a VL comprising the amino acid sequence at least 96% identical to SEQ ID NO:147. In some embodiments, the second antigen-binding site comprises a VH comprising the amino acid sequence at least 97% identical to SEQ ID NO:146 and a VL comprising the amino acid sequence at least 97% identical to SEQ ID NO:147. In some embodiments, the second antigen-binding site comprises a VH comprising the amino acid sequence at least 98% identical to SEQ ID NO:146 and a VL comprising the amino acid sequence at least 98% identical to SEQ ID NO:147. In some embodiments, the second antigen-binding site comprises a VH comprising the amino acid sequence at least 99% identical to SEQ ID NO:146 and a VL comprising the amino acid sequence at least 99% identical to SEQ ID NO:147. In some embodiments, the second antigen-binding site comprises a VH comprising the amino acid sequence of SEQ ID NO:146 and a VL comprising the amino acid sequence of SEQ ID NO:147. In some embodiments, the second antigen-binding site comprises a VH consisting of the amino acid sequence of SEQ ID NO:146 and a VL consisting of the amino acid sequence of SEQ ID NO:147. In some embodiments, the second antigen-binding site comprises a VH comprising the amino acid sequence of SEQ ID NO:144 and a VL comprising the amino acid sequence of SEQ ID NO:145.

In some embodiments, the second antigen-binding site of the protein in the pharmaceutical formulation comprises a single-chain fragment variable (scFv), and the scFv comprises a VH comprising the amino acid sequence of SEQ ID NO:146 and a VL comprising the amino acid sequence of SEQ ID NO:147. In some embodiments, the second antigen-binding site comprises a single-chain fragment variable (scFv), and the scFv comprises an amino acid sequence at least 90%, e.g., at least 91%, at least 92%, at least 93%, at least 94%, at least 95%, at least 96%, at least 97%, at least 98%, at least 99% or 100%, identical to a sequence selected from SEQ ID NOs: 148 and 149. In some embodiments, the second antigen-binding site comprises an scFv, and the scFv comprises an amino acid sequence at least 90%, e.g., at least 91%, at least 92%, at least 93%, at least 94%, at least 95%, at least 96%, at least 97%, at least 98%, at least 99% or 100%, identical to SEQ ID NO:148. In some embodiments, the second antigen-binding site comprises an scFv and the scFv comprises an amino acid sequence of SEQ ID NO:148. In some embodiments, the protein comprises an amino acid sequence of SEQ ID NO:198.

In some embodiments, the present disclosure provides a pharmaceutical composition comprising (a) a protein that comprises: (i) a first antigen-binding site comprising a VH and a VL of an anti-NKG2D antibody, wherein the VH comprises the amino acid sequence of SEQ ID NO:95 and a VL comprises the amino acid sequence of SEQ ID NO:85; (ii) a second antigen-binding site comprising a VH and a VL of an anti-5T4 antibody, wherein the VH comprises the amino acid sequence of SEQ ID NO:146 and a VL comprises the amino acid sequence of SEQ ID NO:147; and (iii) an antibody Fc domain, comprising a first antibody Fc domain polypeptide or a portion thereof sufficient to bind CD16, and a second antibody Fc domain polypeptide or a portion thereof sufficient to bind CD16; (b) a buffer comprising citrate; (c) sucrose; and (d) a polysorbate, wherein the pH of the pharmaceutical formulation is 6.0 to 7.0.

In some embodiments, the present disclosure provides a pharmaceutical composition comprising (a) a protein that comprises: (i) a first antigen-binding site comprising a VH and a VL of an anti-NKG2D antibody, wherein the VH comprises the amino acid sequence of SEQ ID NO:95 and a VL comprises the amino acid sequence of SEQ ID NO:85; (ii) a second antigen-binding site comprising the amino acid sequence of SEQ ID NO:148; and (iii) an antibody Fc domain, comprising a first antibody Fc domain polypeptide or a portion thereof sufficient to bind CD16, and a second antibody Fc domain polypeptide or a portion thereof sufficient to bind CD16; (b) a buffer comprising citrate; (c) sucrose; and (d) a polysorbate, wherein the pH of the pharmaceutical formulation is 6.0 to 7.0.

In some embodiments, the present disclosure provides a vial comprising a pharmaceutical composition comprising (a) a protein that comprises: (i) a first antigen-binding site comprising a VH and a VL of an anti-NKG2D antibody, wherein the VH comprises the amino acid sequence of SEQ ID NO:95 and a VL comprises the amino acid sequence of SEQ ID NO:85; (ii) a second antigen-binding site comprising a VH and a VL of an anti-5T4 antibody, wherein the VH comprises the amino acid sequence of SEQ ID NO:146 and a VL comprises the amino acid sequence of SEQ ID NO:147; and (iii) an antibody Fc domain, comprising a first antibody Fc domain polypeptide or a portion thereof sufficient to bind CD16, and a second antibody Fc domain polypeptide or a portion thereof sufficient to bind CD16; (b) a buffer comprising citrate; (c) sucrose; and (d) a polysorbate, wherein the pH of the pharmaceutical formulation is 6.0 to 7.0.

In some embodiments, the present disclosure provides a vial comprising a pharmaceutical composition comprising (a) a protein that comprises: (i) a first antigen-binding site comprising a VH and a VL of an anti-NKG2D antibody, wherein the VH comprises the amino acid sequence of SEQ ID NO:95 and a VL comprises the amino acid sequence of SEQ ID NO:85; (ii) a second antigen-binding site comprising the amino acid sequence of SEQ ID NO:148; and (iii) an antibody Fc domain, comprising a first antibody Fc domain polypeptide or a portion thereof sufficient to bind CD16, and a second antibody Fc domain polypeptide or a portion thereof sufficient to bind CD16; (b) a buffer comprising citrate; (c) sucrose; and (d) a polysorbate, wherein the pH of the pharmaceutical formulation is 6.0 to 7.0.

In some embodiments, the protein in the pharmaceutical formulation comprises: (a) a first antigen-binding site comprising a heavy chain variable domain (VH) and a light chain variable domain (VL) of an anti-NKG2D antibody; (b) a second antigen-binding site comprising a VH comprising a CDR1, a CDR2, and a CDR3 sequence selected from Table 2 and a VL comprising a CDR1, a CDR2, and a CDR3 sequence selected from Table 2, or a VH comprising a CDR1, a CDR2, and a CDR3 sequence selected from Table 12 and a VL comprising a CDR1, a CDR2, and a CDR3 sequence selected from Table 12; and (c) an antibody Fc domain or a portion thereof sufficient to bind CD16. In some embodiments, the protein in the pharmaceutical formulation comprises: (a) a first antigen-binding site comprising a heavy chain variable domain (VH) and a light chain variable domain (VL) of an anti-NKG2D antibody; (b) a second antigen-binding site comprising a VH comprising a CDR1, a CDR2, and a CDR3 sequence selected from Table 2 and a VL comprising a CDR1, a CDR2, and a CDR3 sequence selected from Table 2, or a VH comprising a CDR1, a CDR2, and a CDR3 sequence selected from Table 12 and a VL comprising a CDR1, a CDR2, and a CDR3 sequence comprising the amino acid sequences of SEQ ID NOs: 189, 190, and 143, respectively; and (c) an antibody Fc domain or a portion thereof sufficient to bind CD16.

In some embodiments, the antibody Fc domain is a human IgG1 antibody Fc domain. In some embodiments, the antibody Fc domain or the portion thereof comprises an amino acid sequence at least 90%, e.g., at least 91%, at least 92%, at least 93%, at least 94%, at least 95%, at least 96%, at least 97%, at least 98%, at least 99% or 100%, identical to SEQ ID NO:118. In some embodiments, at least one polypeptide chain of the antibody Fc domain comprises one or more mutations, relative to SEQ ID NO:118, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system. In some embodiments, at least one polypeptide chain of the antibody Fc domain comprises one or more mutations, relative to SEQ ID NO:118, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, F405L, Y407A, Y407I, Y407V, K409F, K409W, K409D, K409R, T411D, T411E, K439D, and K439E, numbered according to the EU numbering system. In some embodiments, one polypeptide chain of the antibody heavy chain constant region comprises one or more mutations, relative to SEQ ID NO:118, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:118, at one or more positions selected from Q347, Y349, L351, S354, E356, E357, S364, T366, L368, K370, N390, K392, T394, D399, D401, F405, Y407, K409, T411, and K439, numbered according to the EU numbering system. In some embodiments, one polypeptide chain of the antibody heavy chain constant region comprises K360E and K409W substitutions relative to SEQ ID NO:118; and the other polypeptide chain of the antibody heavy chain constant region comprises Q347R, D399V and F405T substitutions relative to SEQ ID NO:118, numbered according to the EU numbering system. In some embodiments, one polypeptide chain of the antibody heavy chain constant region comprises an F405L substitution relative to SEQ ID NO:118; and the other polypeptide chain of the antibody heavy chain constant region comprises a K409R substitution relative to SEQ ID NO:118, numbered according to the EU numbering system. In some embodiments, one polypeptide chain of the antibody heavy chain constant region comprises a Y349C substitution relative to SEQ ID NO:118; and the other polypeptide chain of the antibody heavy chain constant region comprises an S354C substitution relative to SEQ ID NO:118, numbered according to the EU numbering system.

In another aspect, the present disclosure provides a protein comprising: (a) a first polypeptide comprising an amino acid sequence at least 90%, e.g., at least 91%, at least 92%, at least 93%, at least 94%, at least 95%, at least 96%, at least 97%, at least 98%, at least 99%, or 100%, identical to SEQ ID NO:198; (b) a second polypeptide comprising an amino acid sequence at least 90%, e.g., at least 91%, at least 92%, at least 93%, at least 94%, at least 95%, at least 96%, at least 97%, at least 98%, at least 99%, or 100%, identical to SEQ ID NO:199; and (c) a third polypeptide comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:200. In some embodiments, the protein comprises (a) a first polypeptide comprising an amino acid sequence at least 95% identical to SEQ ID NO:198; (b) a second polypeptide comprising an amino acid sequence at least 95% identical to SEQ ID NO:199; and (c) a third polypeptide comprising an amino acid sequence at least 95% identical to SEQ ID NO:200. In some embodiments, the protein comprises (a) a first polypeptide comprising an amino acid sequence at least 96% identical to SEQ ID NO:198; (b) a second polypeptide comprising an amino acid sequence at least 96% identical to SEQ ID NO:199; and (c) a third polypeptide comprising an amino acid sequence at least 96% identical to SEQ ID NO:200. In some embodiments, the protein comprises (a) a first polypeptide comprising an amino acid sequence at least 96% identical to SEQ ID NO:198; (b) a second polypeptide comprising an amino acid sequence at least 96% identical to SEQ ID NO:199; and (c) a third polypeptide comprising an amino acid sequence at least 96% identical to SEQ ID NO:200. In some embodiments, the protein comprises (a) a first polypeptide comprising an amino acid sequence at least 97% identical to SEQ ID NO:198; (b) a second polypeptide comprising an amino acid sequence at least 97% identical to SEQ ID NO:199; and (c) a third polypeptide comprising an amino acid sequence at least 97% identical to SEQ ID NO:200. In some embodiments, the protein comprises (a) a first polypeptide comprising an amino acid sequence at least 98% identical to SEQ ID NO:198; (b) a second polypeptide comprising an amino acid sequence at least 98% identical to SEQ ID NO:199; and (c) a third polypeptide comprising an amino acid sequence at least 98% identical to SEQ ID NO:200. In some embodiments, the protein comprises (a) a first polypeptide comprising an amino acid sequence at least 99% identical to SEQ ID NO:198; (b) a second polypeptide comprising an amino acid sequence at least 99% identical to SEQ ID NO:199; and (c) a third polypeptide comprising an amino acid sequence at least 99% identical to SEQ ID NO:200. In some embodiments, the protein comprises (a) a first polypeptide comprising the amino acid sequence of SEQ ID NO:198; (b) a second polypeptide comprising the amino acid sequence of SEQ ID NO:199; and (c) a third polypeptide comprising the amino acid sequence of SEQ ID NO:200. In certain embodiments of the protein, i) a human NKG2D-binding site is formed by a VH in SEQ ID NO:199 (SEQ ID NO:95) and a VL in SEQ ID NO:200 (SEQ ID NO:85), ii) a human 5T4-binding site is formed by a VH in SEQ ID NO:198 (SEQ ID NO:146) and a VL in SEQ ID NO:198 (SEQ ID NO:147), and iii) a human CD16a-binding site is formed by an Fc binding domain polypeptide in SEQ ID NO:198 and an Fc binding domain polypeptide in SEQ ID NO:199. In certain embodiments of the protein, i) a disulfide bridge is formed between C44 in SEQ ID NO:146 and C100 in SEQ ID NO:147, numbered under the Kabat numbering scheme, ii) a disulfide bridge is formed between C349 in SEQ ID NO:199 and C354 in SEQ ID NO:198, numbered according to the EU numbering system, and iii) a heterodimer is formed between an Fc domain in SEQ ID NO:198 and an Fc domain in SEQ ID NO:199. In some embodiments, the protein is a trispecific antibody. In some embodiments, the trispecific antibody is capable of binding to human NKG2D and human CD16a on the surface of an NK cell and to human 5T4 on the surface of a tumor cell.

In another aspect, the protein in the pharmaceutical formulation or the vial comprises: (i) a first polypeptide comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:198; (ii) a second polypeptide comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:199; and (ii) a third polypeptide comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:200. In some embodiments, the protein comprises (a) a first polypeptide comprising an amino acid sequence at least 95% identical to SEQ ID NO:198; (b) a second polypeptide comprising an amino acid sequence at least 95% identical to SEQ ID NO:199; and (c) a third polypeptide comprising an amino acid sequence at least 95% identical to SEQ ID NO:200. In some embodiments, the protein comprises (a) a first polypeptide comprising an amino acid sequence at least 96% identical to SEQ ID NO:198; (b) a second polypeptide comprising an amino acid sequence at least 96% identical to SEQ ID NO:199; and (c) a third polypeptide comprising an amino acid sequence at least 96% identical to SEQ ID NO:200. In some embodiments, the protein comprises (a) a first polypeptide comprising an amino acid sequence at least 96% identical to SEQ ID NO:198; (b) a second polypeptide comprising an amino acid sequence at least 96% identical to SEQ ID NO:199; and (c) a third polypeptide comprising an amino acid sequence at least 96% identical to SEQ ID NO:200. In some embodiments, the protein comprises (a) a first polypeptide comprising an amino acid sequence at least 97% identical to SEQ ID NO:198; (b) a second polypeptide comprising an amino acid sequence at least 97% identical to SEQ ID NO:199; and (c) a third polypeptide comprising an amino acid sequence at least 97% identical to SEQ ID NO:200. In some embodiments, the protein comprises (a) a first polypeptide comprising an amino acid sequence at least 98% identical to SEQ ID NO:198; (b) a second polypeptide comprising an amino acid sequence at least 98% identical to SEQ ID NO:199; and (c) a third polypeptide comprising an amino acid sequence at least 98% identical to SEQ ID NO:200. In some embodiments, the protein comprises (a) a first polypeptide comprising an amino acid sequence at least 99% identical to SEQ ID NO:198; (b) a second polypeptide comprising an amino acid sequence at least 99% identical to SEQ ID NO:199; and (c) a third polypeptide comprising an amino acid sequence at least 99% identical to SEQ ID NO:200. In some embodiments, the protein comprises (a) a first polypeptide comprising the amino acid sequence of SEQ ID NO:198; (b) a second polypeptide comprising the amino acid sequence of SEQ ID NO:199; and (c) a third polypeptide comprising the amino acid sequence of SEQ ID NO:200. In certain embodiments of the protein, i) a human NKG2D-binding site is formed by a VH in SEQ ID NO:199 (SEQ ID NO:95) and a VL in SEQ ID NO:200 (SEQ ID NO:85), ii) a human 5T4-binding site is formed by a VH in SEQ ID NO:198 (SEQ ID NO:146) and a VL in SEQ ID NO:198 (SEQ ID NO:147), and iii) a human CD16a-binding site is formed by an Fc binding domain polypeptide in SEQ ID NO:198 and an Fc binding domain polypeptide in SEQ ID NO:199. In certain embodiments of the protein, i) a disulfide bridge is formed between C44 in SEQ ID NO:146 and C100 in SEQ ID NO:147, numbered under the Kabat numbering scheme, ii) a disulfide bridge is formed between C349 in SEQ ID NO:199 and C354 in SEQ ID NO:198, numbered according to the EU numbering system, and iii) a heterodimer is formed between an Fc domain in SEQ ID NO:198 and an Fc domain in SEQ ID NO:199. In some embodiments, the protein is a trispecific antibody. In some embodiments, the trispecific antibody is capable of binding to human NKG2D and human CD16a on the surface of an NK cell and to human 5T4 on the surface of a tumor cell.

In another aspect, the present disclosure provides a pharmaceutical composition comprising a protein provided herein and a pharmaceutically acceptable carrier.

In another aspect, the present disclosure provides a cell comprising one or more nucleic acids encoding a protein provided herein.

In another aspect, the present disclosure provides a method of enhancing tumor cell death, the method comprising exposing the tumor cell and a natural killer cell to an effective amount of the protein provided herein or the pharmaceutical composition provided herein. In another aspect, provided the use of a protein provided herein or a pharmaceutical composition provided herein for enhancing tumor cell death by exposing the tumor cell and a natural killer cell to an effective amount of the protein or a pharmaceutical composition comprising such protein. In another aspect, provided is a protein provided herein or a pharmaceutical composition provided herein for use in enhancing tumor cell death by exposing the tumor cell and a natural killer cell to an effective amount of the protein or a pharmaceutical composition comprising such protein.

In another aspect, the present disclosure provides a method of enhancing cancer-associated fibroblast (CAF) cell death, the method comprising exposing the CAF and a natural killer cell to an effective amount of the protein provided herein or the pharmaceutical composition provided herein. In another aspect, provided is the use of a protein provided herein or a pharmaceutical composition provided herein for enhancing cancer-associated fibroblast (CAF) cell death by exposing the CAF and a natural killer cell to an effective amount of the protein or a pharmaceutical composition comprising such protein. In another aspect, provided is a protein provided herein or a pharmaceutical composition provided herein for use in enhancing cancer-associated fibroblast (CAF) cell death by exposing the CAF and a natural killer cell to an effective amount of the protein or a pharmaceutical composition comprising such protein.

In another aspect, the present disclosure provides a method of treating cancer, the method comprising administering to a subject in need thereof an effective amount of the protein provided herein or the pharmaceutical composition provided herein. In another aspect, the provided is the use of a protein provided herein or a pharmaceutical composition provided herein for treating cancer. In another aspect, the provided is a protein provided herein or a pharmaceutical composition provided herein for use in treating cancer. In some embodiments, the cancer is selected from the group consisting of colorectal cancer, ovarian cancer, non-small cell lung cancer, renal cancer, breast cancer (e.g., hormone receptor positive (HR+) breast cancer), endometrial cancer, squamous cell carcinoma, head and neck squamous cell carcinoma, uterine cancer, pancreatic cancer, mesothelioma, and gastric cancer. In some embodiments, the cancer is a metastatic cancer. In some embodiments, the subject is refractory to chemotherapy. In some embodiments, wherein the method increases overall survival and/or progression free survival in the subject.

In some embodiments, 5T4 is expressed by cancer cells. In some embodiments, 5T4 is expressed by cancer-associated fibroblasts. In some embodiments, 5T4 is expressed at high levels relative to normal cells. In some embodiments, 5T4 is expressed at low levels relative to normal cells.

In some embodiments, the protein provided herein is a purified protein. In some embodiments, the trispecific antibody provided herein is a purified trispecific antibody. In some embodiments, the protein or trispecific antibody is purified using a method selected from the group consisting of: centrifugation, depth filtration, cell lysis, homogenization, freeze-thawing, affinity purification, gel filtration, ion exchange chromatography, hydrophobic interaction exchange chromatography, and mixed-mode chromatography.

### BRIEF DESCRIPTION OF THE DRAWINGS

FIG. 1 is a representation of a heterodimeric, multispecific antibody, e.g., a trispecific binding protein (TriNKET®). Each arm can represent either the NKG2D binding domain, or the 5T4 binding domain. In some embodiments, the NKG2D binding domain and the 5T4 binding domains can share a common light chain.

FIGS. 2A-2E illustrate five exemplary formats of a multispecific binding protein, e.g., a trispecific binding protein (TriNKET®). As shown in FIG. 2A, either the NKG2D-binding domain or the 5T4 binding domain can take the scFv format (left arm). An antibody that contains a NKG2D targeting scFv, a 5T4 targeting Fab fragment, and a heterodimerized antibody constant region is referred herein as the F3-TriNKET®. An antibody that contains a 5T4 targeting scFv, a NKG2D targeting Fab fragment, and a heterodimerized antibody constant region/domain that binds CD16 is referred herein as the F3′-TriNKET® (FIG. 2E). As shown in FIG. 2B, both the NKG2D binding domain and 5T4 binding domain can take the scFv format. FIGS. 2C to 2D are illustrations of an antibody with three antigen-binding sites, including two antigen-binding sites that bind 5T4, and the NKG2D-binding site fused to the heterodimerized antibody constant region. These antibody formats are referred herein as F4-TriNKET®. FIG. 2C illustrates that the two 5T4 binding sites are in the Fab fragment format, and the NKG2D binding site in the scFv format. FIG. 2D illustrates that the 5T4 binding sites are in the scFv format, and the NKG2D binding site is in the scFv format. FIG. 2E represents a trispecific antibody (TriNKET®) that contains a 5T4 targeting scFv, a NKG2D targeting Fab fragment, and a heterodimerized antibody constant region/domain (“CD domain”) that binds CD16. The antibody format is referred herein as F3′-TriNKET®. In certain exemplary multispecific binding proteins, heterodimerization mutations on the antibody constant region include K360E and K409W on one constant domain; and Q347R, D399V and F405T on the opposite constant domain (shown as a triangular lock-and-key shape in the CD domains). The bold bar between the heavy and the light chain variable domains of the Fab fragments represents a disulfide bond.

FIG. 3 is a representation of a TriNKET® in the Triomab form, which is a trifunctional, bispecific antibody that maintains an IgG-like shape. This chimera consists of two half antibodies, each with one light and one heavy chain, that originate from two parental antibodies. Triomab form may be a heterodimeric construct containing ½ of rat antibody and ½ of mouse antibody.

FIG. 4 is a representation of a TriNKET® in the KiH Common Light Chain form, which involves the knobs-into-holes (KIHs) technology. KiH is a heterodimer containing 2 Fab fragments binding to target 1 and 2, and an Fc stabilized by heterodimerization mutations. TriNKET® in the KiH format may be a heterodimeric construct with 2 Fab fragments binding to target 1 and target 2, containing two different heavy chains and a common light chain that pairs with both heavy chains.

FIG. 5 is a representation of a TriNKET® in the dual-variable domain immunoglobulin (DVD-Ig™) form, which combines the target-binding domains of two monoclonal antibodies via flexible naturally occurring linkers, and yields a tetravalent IgG-like molecule. DVD-Ig™ is a homodimeric construct where variable domain targeting antigen 2 is fused to the N-terminus of a variable domain of Fab fragment targeting antigen 1. DVD-Ig™ form contains normal Fc.

FIG. 6 is a representation of a TriNKET® in the Orthogonal Fab fragment interface (Ortho-Fab) form, which is a heterodimeric construct that contains 2 Fab fragments binding to target 1 and target 2 fused to Fc. Light chain (LC)-heavy chain (HC) pairing is ensured by orthogonal interface. Heterodimerization is ensured by mutations in the Fc.

FIG. 7 is a representation of a TriNTKET® in the 2-in-1 Ig format.

FIG. 8 is a representation of a TriNKET® in the ES form, which is a heterodimeric construct containing two different Fab fragments binding to target 1 and target 2 fused to the Fc. Heterodimerization is ensured by electrostatic steering mutations in the Fc.

FIG. 9 is a representation of a TriNKET® in the Fab Arm Exchange form: antibodies that exchange Fab fragment arms by swapping a heavy chain and attached light chain (half-molecule) with a heavy-light chain pair from another molecule, resulting in bispecific antibodies. Fab Arm Exchange form (cFae) is a heterodimer containing 2 Fab fragments binding to target 1 and 2, and an Fc stabilized by heterodimerization mutations.

FIG. 10 is a representation of a TriNKET® in the SEED Body form, which is a heterodimer containing 2 Fab fragments binding to target 1 and 2, and an Fc stabilized by heterodimerization mutations.

FIG. 11 is a representation of a TriNKET® in the LuZ-Y form, in which a leucine zipper is used to induce heterodimerization of two different HCs. The LuZ-Y form is a heterodimer containing two different scFabs binding to target 1 and 2, fused to Fc. Heterodimerization is ensured through leucine zipper motifs fused to C-terminus of Fc.

FIG. 12 is a representation of a TriNKET® in the Cov-X-Body form.

FIGS. 13A-13B are representations of TriNKETs® in the Kλ-Body forms, which are heterodimeric constructs with two different Fab fragments fused to Fc stabilized by heterodimerization mutations: one Fab fragment targeting antigen 1 contains kappa LC, and the second Fab fragment targeting antigen 2 contains lambda LC. FIG. 13A is an exemplary representation of one form of a Kλ-Body; FIG. 13B is an exemplary representation of another Kλ-Body.

FIG. 14 is a representation of an OAsc-Fab heterodimeric construct that includes Fab fragment binding to target 1 and scFab binding to target 2, both of which are fused to the Fc domain. Heterodimerization is ensured by mutations in the Fc domain.

FIG. 15 is a representation of a DuetMab, which is a heterodimeric construct containing two different Fab fragments binding to antigens 1 and 2, and an Fc that is stabilized by heterodimerization mutations. Fab fragments 1 and 2 contain differential S-S bridges that ensure correct light chain and heavy chain pairing.

FIG. 16 is a representation of a CrossmAb, which is a heterodimeric construct with two different Fab fragments binding to targets 1 and 2, and an Fc stabilized by heterodimerization mutations. CL and CH1 domains, and VH and VL domains are switched, e.g., CH1 is fused in-line with VL, and CL is fused in-line with VH.

FIG. 17 is a representation of a Fit-Ig, which is a homodimeric construct where Fab fragment binding to antigen 2 is fused to the N-terminus of HC of Fab fragment that binds to antigen 1. The construct contains wild-type Fc.

FIGS. 18A-18C are graphs showing binding and lysis of 5T4⁺ H1975 cells by 5T4-binding TriNKETs® with 5T4 binding sites of the indicated murine antibody clones. FIG. 18A shows concentration curves of binding of 5T4-TriNKETs® to H1975 cells. FIG. 18B shows lysis of H1975 cells induced by KHYG-CD16V cells incubated in the presence of 5T4-TriNKETs® over varying concentrations. FIG. 18C shows lysis of H1975 cells induced by KHYG-CD16V cells incubated in the presence of indicated 08E06-derived 5T4-TriNKETs® over varying concentrations.

FIGS. 19A-19H are graphs showing surface plasmon resonance (SPR) of multispecific binding proteins. FIG. 19A shows binding of AB1310/AB1783-TriNKET® to human 5T4 at pH 7.4. FIG. 19B shows binding of AB0064-TriNKET® to human 5T4 at pH 7.4. FIG. 19C shows binding of AB0064-TriNKET® to cynomolgus 5T4 at pH 7.4. FIG. 19D shows binding of AB0063-TriNKET® to human 5T4 at pH 7.4. FIG. 19E shows binding of AB0063 to cynomolgus 5T4 at pH 7.4. FIG. 19F shows binding of AB1310/AB1783-TriNKET® to human NKG2D at pH 7.4. FIG. 19G shows binding of AB1310/AB1783-TriNKET® to human NKG2D at pH 7.4. FIG. 19H shows binding of AB1310/AB1783-TriNKET® to human CD16a at pH 7.4.

FIGS. 20A-20C are graphs showing concentration curves showing saturation of binding of AB1310/AB1783-TriNKET® and the parental antibody 10F10 to 5T4-expressing cells. FIG. 20A shows binding to KYSE-30 cells. FIG. 20B shows binding to H292 cells. FIG. 20C shows binding to H2172 cells.

FIGS. 21A-21F are graphs showing activation of immune cells as measured by lysis of tumor cells or cytokine release induced by NK cells, T cells, or macrophages incubated in the presence of indicated 5T4-TriNKETs® over varying concentrations. FIG. 21A shows lysis of H292 cells induced by V/F NK cells grown in the presence of AB1310/AB1783-TriNKET®. FIG. 21B shows lysis of H292 cells induced by F/F NK cells grown in the presence of AB1310/AB1783-TriNKET®. FIG. 21C shows lysis of 786-0 cells induced by stimulated CD8+ T cells grown in the presence of AB1310/AB1783-TriNKET®. FIG. 21D shows interferon-gamma (IFNγ) release by H1975 cells induced by primary NK cells grown in the presence of AB1310/AB1783-TriNKET®. FIG. 21E shows phagocytosis of H292 cells by primary M0 macrophages grown in the presence of AB1310/AB1783-TriNKET®. FIG. 21F shows phagocytosis of KYSE-30 esophageal squamous cell carcinoma (EsoSCC) cells by primary M0 macrophages grown in the presence of AB1310/AB1783-TriNKET®.

FIGS. 22A-22D are graphs showing binding or lysis of cancer-associated fibroblasts (CAFs). FIG. 22A is a concentration curve showing saturation of binding of AB1310/AB1783-TriNKET® to CAFs. FIG. 22B is a plot showing observed binding EC₅₀ values of AB1310/AB1783-TriNKET® to tumor cell lines and primary CAFs. FIG. 22C shows lysis of CAFs induced by V/F NK cells in the presence of AB1310/AB1783-TriNKET®. FIG. 22D shows lysis of CAFs induced by F/F NK cells in the presence of AB1310/AB1783-TriNKET®.

FIG. 23 shows graphs of a polyspecificity assay showing AB1310/AB1783-TriNKET® (left panels) or controls (center and right panels) in the absence (top panels) or presence (bottom panels) of poly-specificity reagent (PSR).

FIGS. 24A-24H are graphs summarizing the manufacturability of AB1310/AB1783-TriNKET®. FIG. 24A is a chromatogram showing size-exclusion chromatography (SEC) analysis of AB1310/AB1783-TriNKET®. FIG. 24B is a graph showing non-reduced capillary electrophoresis (NR-CE) of AB1310/AB1783-TriNKET®. FIG. 24C is a graph showing reduced capillary electrophoresis (R-CE) of AB1310/AB1783-TriNKET®. FIG. 24D is a graph showing mass spectrometry analysis of AB1310/AB1783-TriNKET®. FIG. 24E is a graph showing capillary isoelectric focusing (cIEF) analysis of AB1310/AB1783-TriNKET®. FIG. 24F is a graph showing differential scanning calorimetry (DSC) analysis of AB1310/AB1783-TriNKET®. FIG. 24G is a graph showing hydrophobic interaction chromatography (HIC) analysis of AB1310/AB1783-TriNKET®. FIG. 24H is a graph comparing HIC analysis of AB1310/AB1783-TriNKET® relative to known benchmark monoclonal antibodies.

FIGS. 25A-25G are graphs summarizing AB1310/AB1783-TriNKET® stability. FIG. 25A is a graph summarizing SEC analysis of AB1310/AB1783-TriNKET® after 78 hr incubations at various temperatures. FIG. 25B is a graph showing DSC analysis of AB1310/AB1783-TriNKET® in PBS buffer at pH 7.4. FIG. 25C is a graph showing DSC analysis of AB1310/AB1783-TriNKET® in HST buffer at pH 6.0. FIG. 25D is a graph summarizing SEC analysis of AB1310/AB1783-TriNKET® after incubation under indicated conditions. FIG. 25E is a graph showing NR-CE analysis of AB1310/AB1783-TriNKET® after incubation under indicated conditions. FIG. 25F is a graph showing R-CE of AB1310/AB1783-TriNKET® after incubation under indicated conditions. FIG. 25G is a graph showing lysis of H292 cells induced by KHYG-CD16V NK cells incubated in the presence of AB1310/AB1783-TriNKETs® over varying concentrations and after incubation under indicated conditions.

FIGS. 26A-26D are graphs showing binding (fold over background (FOB)) of various concentrations of 10F10 (FIG. 26A and FIG. 26C), 11F09 (FIG. 26B and FIG. 26D), and mutants thereof produced via humanization and sequence liability correction to 5T4+H1975 cells. FIG. 26E and FIG. 26F are graphs showing binding (fold over background (FOB)) of various concentrations of humanized 5T4 binders to 5T4-expressing tumor cells.

FIGS. 27A-27C are protein assays showing binding of AB1310/AB1783-TriNKET® to 5T4 (TPBG) and NKG2D-DAP10 (KLRK1+HCST) for TriNKET® (FIG. 27A), parental mAb (FIG. 27B), and Fc-silent TriNKET® (FIG. 27C).

FIGS. 28A-28B are graphs demonstrating co-engagement of AB1310/AB1783-TriNKET® 5T4 and NKG2D targeting arms, regardless of binding to 5T4 first (FIG. 28A) or NKG2D first (FIG. 28B).

FIG. 29 is a sensorgram depicting AB1310/AB1783-TriNKET® binding both NKG2D and CD16.

FIGS. 30A-30B are graphs showing NK cell activation by AB1310/AB1783-TriNKET® following co-culture with KYSE-30 cells for human (FIG. 30A) and cynomolgus monkey (FIG. 30B) NK cells.

FIGS. 31A-31D are graphs showing effector cell-mediated killing of 5T4⁺ cell lines by AB1310-TriNKET® compared to control. FIG. 31A shows NK cell-mediated killing of KYSE-30 cells. FIG. 31B shows NK cell-mediated killing of H292 cells. FIG. 31C shows peripheral blood mononuclear cell (PBMC)-mediated killing of KYSE-30 cells. FIG. 31D shows PBMC-mediated killing of H292 cells.

FIGS. 32A-32C show SEC results of exemplary formulations including AB1310/AB1783-TriNKET®. FIG. 32A is a chromatogram of AB1310/AB1783-TriNKET® in formulations incubated at 50° C. for 6 days. FIG. 32B is a chromatogram of AB1310/AB1783-TriNKET® in formulations incubated at 40° C. for 21 days. FIG. 32C is a graph showing changes in percent monomer of AB1310/AB1783-TriNKET® in formulations with indicated pH at indicated storage temperatures.

FIGS. 33A-33C are graphs showing changes in charged species as measured by cIEF in acidic (FIG. 33A), neutral (FIG. 33B), and basic (FIG. 33C) regions of the electropherograms of formulations including AB1310/AB1783-TriNKET® at indicated pH levels.

FIGS. 34A-34C show changes in purity (FIG. 34A), number of fragments (FIG. 34B), and high molecular weight species (FIG. 34C) of formulations including AB1310/AB1783-TriNKET® at indicated pH levels as measured by NR-CE.

FIG. 35 shows a percent change in monomers as observed by SEC of AB1310/AB1783-TriNKET® in indicated formulations.

FIG. 36 shows a percent change in peaks in the neutral region of the electropherograms as observed by cIEF of AB1310/AB1783-TriNKET® in indicated formulations.

FIGS. 37A and 37B show purity of AB1310/AB1783-TriNKET® in indicated formulations as measured by R-CE (FIG. 37A) and NR-CE (FIG. 37B).

FIGS. 38A-38D are graphs of dynamic light scattering (DLS) results of AB1310/AB1783-TriNKET® in indicated formulations. FIG. 38A shows DLS results for a formulation containing histidine buffer and polysorbate 80. FIG. 38B shows DLS results for a formulation containing histidine buffer with no polysorbate 80. FIG. 38C shows DLS results for a formulation containing citrate buffer and polysorbate 80. FIG. 38D shows DLS results for a formulation containing citrate buffer with no polysorbate 80.

FIGS. 39A-39C are graphs showing % monomer over time of AB1310/AB1783-TriNKET® in formulations containing indicated concentrations of sucrose (in % w/v) as measured by SEC. FIG. 39A is a graph showing % monomer over time of AB1310/AB1783-TriNKET® in a formulation containing 6% (w/v) sucrose incubated at 30° C. FIG. 39B is a graph showing % monomer over time of AB1310/AB1783-TriNKET® in formulations containing 3%, 6%, or 9% (w/v) sucrose incubated at 40° C. FIG. 39C is a graph showing % monomer over time of AB1310/AB1783-TriNKET® in formulations containing 3%, 6%, or 9% (w/v) sucrose incubated at 50° C.

FIGS. 40A-40C are graphs showing % main peak in the neutral region of the electropherogram over time of AB1310/AB1783-TriNKET® in formulations containing indicated concentrations of sucrose (in % w/v) as measured by cIEF. FIG. 40A is a graph showing % main peak in the neutral region of the electropherogram over time of AB1310/AB1783-TriNKET® in a formulation containing 6% (w/v) sucrose incubated at 30° C. FIG. 40B is a graph showing % main peak in the neutral region of the electropherogram over time of AB1310/AB1783-TriNKET® in formulations containing 3%, 6%, or 9% (w/v) sucrose incubated at 40° C. FIG. 40C is a graph showing % main peak in the neutral region of the electropherogram over time of AB1310/AB1783-TriNKET® in formulations containing 3%, 6%, or 9% (w/v) sucrose incubated at 50° C.

FIG. 41 shows the combined results of FIG. 39B and FIG. 40B over the indicated sucrose concentrations (in % w/v).

FIGS. 42A-42B illustrate lysis of BT-474 (breast cancer (BRCC); left panel) or FaDu (head and neck squamous cell carcinoma (HNSCC); right panel) tumor cells measured in co-culture with primed primary human CD8+ T cells. CD8+ T cells were isolated from Concanavalin A (Con A) and interleukin (IL)-2-activated peripheral blood mononuclear cells (PBMCs) and then expanded and primed for 9 days with IL-15. Each point and error bars represent mean and standard deviation (SD), respectively, of % inhibition from 4 total images from duplicate co-culture wells with a different test article. E:T no-treatment background lysis is marked with a dotted line. Dose-response curves were fit with a nonlinear 4-parameter regression model in GraphPad Prism. The plots depicted are data from a single donor, representative of data from 3 healthy T-cell donors.

FIGS. 43A-43D illustrate long-term lysis of 5T4-expressing tumor cell lines KYSE-30 (esophageal squamous cell (EsoSCC) carcinoma; FIGS. 43A-43B) and NCI-H292 (non-small-cell lung cancer (NSCLC); (FIGS. 43C-43D)) measured in co-culture with overnight-rested NK cells with only low-affinity CD16a variant (158FF or F/F; left column) or with some presence of high-affinity CD16a polymorphism F158V (158VF or V/F; right column) over 72 hours. Rested primary human NK cells were added for a 5:1 effector-to-target cell ratio (E:T) to wells that had been pre-seeded for 4 hours with 3000 tumor cells/well transfected to stably express NucLight™ Green. AB1310/AB1783-TriNKET® (circles) and parental mAb (squares) were dose titrated from 100 nM to 0.25 pM or 100 nM to 0.05 pM, in 1:5 dilutions. Green fluorescent images were taken to assess the growth and survival of tumor cells over time using an Incucyte® Live-Cell Imager. % Inhibition at 72 hours was calculated by comparing treatment wells to no-treatment wells, after normalization to the initial scan to control for variability in cell seeding in the well imaging area. Each point and error bars represent mean and standard deviation (SD), respectively, of % inhibition from 4 total images from duplicate co-culture wells with a different test article. Dose-response curves were fit with a nonlinear 4-parameter regression model in GraphPad Prism. The plots shown are data from a single donor of a given CD16a genotype, representative of data from 1-2 healthy human NK cell donors of the same CD16a genotype.

### DETAILED DESCRIPTION

The present application provides multispecific binding proteins that bind the NKG2D receptor and CD16 receptor on natural killer cells, and 5T4 on a cancer cell. In some embodiments, the multispecific binding proteins further include an additional antigen-binding site that binds 5T4. The application also provides pharmaceutical compositions comprising such multispecific binding proteins, and therapeutic methods using such multispecific binding proteins and pharmaceutical compositions, for purposes such as treating cancer. Various aspects of the multispecific binding proteins described in the present application are set forth below in sections; however, aspects of the multispecific binding proteins described in one particular section are not to be limited to any particular section.

To facilitate an understanding of the present application, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

The amino acid sequence boundaries of a CDR can be determined by one of skill in the art using any of a number of known numbering schemes, including those described by Kabat et al., J. Biol. Chem. 252, 6609-6616 (1977) and Kabat et al., Sequences of protein of immunological interest. (1991) (“Kabat” numbering scheme); Chothia et al., J. Mol. Biol. 196:901-917 (1987), Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegger and Plückthun, J. Mol. Biol., 2001, 309:657-70 (“AHo” or “Honegger” numbering scheme); each of which is incorporated by reference in its entirety. CDRs may be assigned, for example, using antibody numbering software, such as Abnum, available at www.bioinforg.uk/abs/abnum/, and described in Abhinandan and Martin, Immunology, 2008, 45:3832-3839, incorporated by reference in its entirety.

The term “protein” as used herein means a macromolecule that comprises one or more chains of amino acids. Such a chain of amino acids may be referred to as a polypeptide, which is a continuous, unbranched chain of amino acids linked by peptide bonds. Accordingly, a protein may include a single polypeptide or multiple polypeptides.

The term “tumor-associated antigen” as used herein means any antigen including but not limited to a protein, glycoprotein, ganglioside, carbohydrate, or lipid that is associated with cancer. Such antigen can be expressed on malignant cells or in the tumor microenvironment such as on tumor-associated blood vessels, extracellular matrix, mesenchymal stroma, or immune infiltrates. In certain embodiments of the present disclosure, the term “tumor-associated antigen” refers to 5T4, which is targeted by the second and/or the additional antigen-binding site present in a multispecific binding proteins of the present disclosure. It is understood, however, that 5T4 may also be associated with diseases and disorders that are not tumor or cancer.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present application) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

With respect to treatment of cancer, a 5T4-targeting TriNKET® (anti-5T4×NKG2D×CD16 multispecific molecule), as described herein, optionally, with one or more additional therapeutic agents, as described herein, can (i) reduce the number of diseased cells; (ii) reduce tumor size; (iii) inhibit, retard, slow to some extent, and preferably stop the diseased cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with cancer or myeloproliferative disease. In some embodiments, a 5T4-targeting TriNKET® (anti-5T4×NKG2D×CD16 multispecific molecule), as described herein, optionally, with one or more additional therapeutic agents, as described herein, can (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

An “increased” or “enhanced” amount (e.g., with respect to cancer cell proliferation or expansion, antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least²⁰⁰%, at least 500%, or at least 1000% of an amount or level described herein. In various embodiments, tumor burden is determined using linear dimensional methods (e.g. Response Evaluation Criteria in Solid Tumors (RECIST) v1.1 (Eisenhauer, et al., Eur J Cancer. (2009) 45(2):228-47). In various embodiments, tumor burden is determined using volumetric analysis (e.g., positron emission tomography (PET)/computed tomography (CT) scan). See, e.g., Paydary, et al., Mol Imaging Biol. (2019) 21(1):1-10; Li, et al., AJR Am J Roentgenol. (2021) 217(6):1433-1443; and Kerner, et al., EJNMMI Res. (2016) December; 6(1):33.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence or recurrence of a tumor, e.g., a relapse after remission.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound described in the present application which, upon administration to a subject, is capable of providing a compound described in this application or an active metabolite or residue thereof. As is known to those of skill in the art, “salts” of the compounds described in the present application may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, though not in themselves pharmaceutically acceptable, may be employed in the preparation of salts useful as intermediates in obtaining the compounds described in the application and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds described in the present application compounded with a suitable cation such as Na⁺, NH₄⁺, and NW₄⁺ (wherein W is a C₁₋₄ alkyl group), and the like.

For therapeutic use, salts of the compounds described in the present application are contemplated as being pharmaceutically acceptable. However, salts of acids and bases that are non-pharmaceutically acceptable may also find use, for example, in the preparation or purification of a pharmaceutically acceptable compound.

As used herein, 5T4 (also known as Trophoblast glycoprotein, TPBG, Wnt-activated Inhibitory Factor 1, WAIF1, M6P1, and 5T4AG) refers to the protein of Uniprot Accession No. Q13641 and related isoforms and orthologs. The NCBI Gene ID for 5T4 is 7162.

In certain embodiments, low expression of 5T4 refers to about 5,000 to about 20,000 copies/cell, e.g., about 5,000 to about 10,000, about 5,000 to about 15,000, or about 5,000 to about 20,000. In certain embodiments, high expression of 5T4 refers to about 40,000 to about 60,000 copies/cell, e.g., about 40,000 to about 60,000, about 45,000 to about 60,000, about 50,000 to about 55,000, or about 55,000 to about 60,000 copies/cell.

As used herein, NKG2D (also known as Killer Cell Lectin Like Receptor K1, D12S2489E, CD314, KLR, Killer Cell Lectin-Like Receptor Subfamily K, Member 1, NKG2-D Type II Integral Membrane Protein, NKG2-D-Activating NK Receptor, and NK Cell Receptor D) refers to the protein of Uniprot Accession No. P26718 and related isoforms and orthologs. The NCBI Gene ID for NKG2D is 22914.

As used herein, the terms “specific binding,” “specifically binds to,” “specific for,” “selectively binds,” and “selective for,” with regard to the binding of a multispecific binding protein or antigen-binding fragment as described herein to a target molecule, a particular antigen (e.g., a polypeptide target), or an epitope on a particular antigen, mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the multispecific binding protein or antigen-binding fragment to the target molecule is competitively inhibited by the control molecule. A multispecific binding protein or antigen-binding fragment as described herein that “specifically binds to” or is “specific for” a particular polypeptide or an epitope on a particular polypeptide is one that binds to that particular polypeptide or epitope on a particular polypeptide without substantially binding to any other polypeptide or polypeptide epitope. In some instances, the multispecific binding protein or antigen-binding fragment as described herein specifically binds to an antigen, e.g., a polypeptide target, with dissociation constant (K_{D}) as described herein, for example, in the form of an scFv, Fab, or other form of a multispecific binding protein measured at a temperature of about 4° C., 25° C., 37° C., or 42° C. Affinities of a multispecific binding protein or antigen-binding fragment as described herein can be readily determined using conventional techniques, for example, those described by Scatchard et al., Ann. N. Y. Acad. Sci. USA, 51:660 (1949), ELISA assays, biolayer interferometry (BLI) assays, and surface plasmon resonance (SPR) assays. Binding properties of a multispecific binding protein or antigen-binding fragment as described herein to antigens, cells, or tissues thereof may generally be determined and assessed using immunodetection methods including, for example, immunofluorescence-based assays, such as immuno-histochemistry (IHC) and/or fluorescence-activated cell sorting (FACS). Generally, but not necessarily, reference to “binding” means “specific binding.”

Throughout the description, where compositions are described as having, including, or comprising specific components, or where processes and methods are described as having, including, or comprising specific steps, it is contemplated that, additionally, there are compositions described in the present application that consist essentially of, or consist of, the recited components, and that there are processes and methods according to the present application that consist essentially of, or consist of, the recited processing steps.

As a general matter, compositions specifying a percentage are by weight unless otherwise specified. Further, if a variable is not accompanied by a definition, then the previous definition of the variable controls.

### I. Proteins

The present application provides multispecific binding proteins that bind to the NKG2D receptor and CD16 receptor on natural killer cells, and 5T4 expressed on a cancer cell and/or a cancer-associated fibroblast. The multispecific binding proteins are useful in the pharmaceutical compositions and therapeutic methods described herein. Binding of the multispecific binding proteins to the NKG2D receptor and CD16 receptor on a natural killer cell enhances the activity of the natural killer cell toward destruction of tumor cells expressing 5T4 antigen. Binding of the multispecific binding proteins to 5T4-expressing cells brings the cancer cells into proximity with the natural killer cell, which facilitates direct and indirect destruction of the tumor cells by the natural killer cell. Multispecific binding proteins that bind NKG2D, CD16, and another target are disclosed in International Application Publication Nos. WO2018148445 and WO2019157366, which are incorporated herein by reference in their entireties for all purposes. Further description of some exemplary multispecific binding proteins is provided below.

The first component of the multispecific binding protein is an antigen-binding site of an anti-NKG2D antibody that binds to NKG2D. NKG2D is a receptor that can be found on NKG2D-expressing cells, which can include but are not limited to NK cells, 76 T cells and CD8⁺ αβ T cells. Upon NKG2D binding, the multispecific binding proteins may block natural ligands, such as ULBP6 and MICA, from binding to NKG2D and activating NK cells.

The second component of the multispecific binding protein is an antigen-binding site of an anti-5T4 antibody that binds to 5T4. The 5T4-expressing cells may be found, for example, in colorectal cancer, ovarian cancer, non-small cell lung cancer, renal cancer, breast cancer (e.g., hormone receptor positive (HR+) breast cancer), endometrial cancer, squamous cell carcinoma, head and neck squamous cell carcinoma, uterine cancer, pancreatic cancer, mesothelioma, and gastric cancer.

The third component of the multispecific binding proteins is an antibody Fc domain or a portion thereof sufficient to bind CD16, or an antigen-binding site that binds to cells expressing CD16. CD16 is an Fc receptor found on the surface of leukocytes including natural killer cells, macrophages, neutrophils, eosinophils, mast cells, and follicular dendritic cells.

In some embodiments, first and second components of the multispecific binding protein take the form of an antibody fragment described herein. Accordingly, the antigen-binding site having the VH and VL of the anti-NKG2D antibody and the antigen-binding site having the VH and VL of the anti-5T4 antibody can be independently any one of the antibody fragments described herein, In some embodiments, the antigen-binding site having the VH and the VL of the anti-NKG2D antibody is a Fab fragment, and the antigen-binding site having the VH and the VL of the anti-5T4 antibody is an scFv. In some embodiments, the antigen-binding site having the VH and the VL of the anti-NKG2D antibody is an scFv, and the second antigen-binding site comprising the VH and the VL of the anti-5T4 antibody is a Fab fragment.

An additional antigen-binding site of the multispecific binding proteins may also bind 5T4. In certain embodiments, the first antigen-binding site that binds NKG2D is an scFv, and the second and the additional antigen-binding sites that bind 5T4 are each a Fab fragment. In certain embodiments, the first antigen-binding site that binds NKG2D is an scFv, and the second and the additional antigen-binding sites that bind 5T4 are each an scFv. In certain embodiments, the first antigen-binding site that binds NKG2D is a Fab fragment, and the second and the additional antigen-binding sites that bind 5T4 are each an scFv. In certain embodiments, the first antigen-binding site that binds NKG2D is a Fab, and the second and the additional antigen-binding sites that bind 5T4 are each a Fab fragment. In some embodiments, the Fab fragment that binds NKG2D is not positioned between an antigen-binding site and an Fc or the portion thereof. In some embodiments, no Fab fragment that bind 5T4 is positioned between an antigen-binding site and an Fc or the portion thereof.

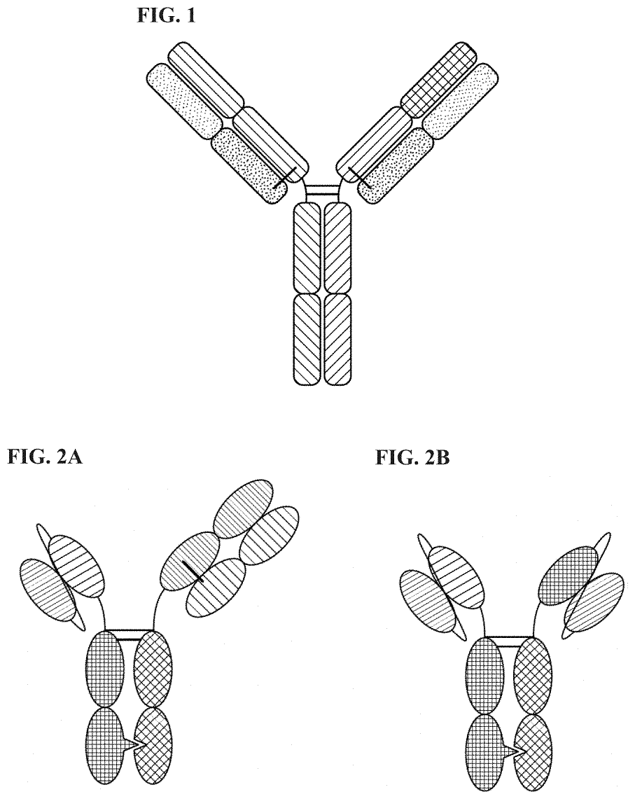

The multispecific binding proteins described herein can take various formats. For example, one format is a heterodimeric, multispecific antibody including a first immunoglobulin heavy chain, a first immunoglobulin light chain, a second immunoglobulin heavy chain and a second immunoglobulin light chain (FIG. 1). The first immunoglobulin heavy chain includes a first Fc (hinge-CH2-CH3) domain, a first heavy chain variable domain and optionally a first CH1 heavy chain domain. The first immunoglobulin light chain includes a first light chain variable domain and optionally a first light chain constant domain. The first immunoglobulin light chain, together with the first immunoglobulin heavy chain, forms an antigen-binding site that binds NKG2D. The second immunoglobulin heavy chain comprises a second Fc (hinge-CH2-CH3) domain, a second heavy chain variable domain and optionally a second CH1 heavy chain domain. The second immunoglobulin light chain includes a second light chain variable domain and optionally a second light chain constant domain. The second immunoglobulin light chain, together with the second immunoglobulin heavy chain, forms an antigen-binding site that binds 5T4. In some embodiments, the first Fc domain and second Fc domain together are able to bind to CD16 (FIG. 1). In some embodiments, the first immunoglobulin light chain is identical to the second immunoglobulin light chain.

The antigen-binding sites may each incorporate an antibody heavy chain variable domain and an antibody light chain variable domain (e.g., arranged as in an antibody, or fused together to form an scFv), or one or more of the antigen-binding sites may be a single domain antibody, such as a V_{H}H antibody like a camelid antibody or a V_{NAR} antibody like those found in cartilaginous fish.

In some embodiments, the second antigen-binding site incorporates a light chain variable domain having an amino acid sequence identical to the amino acid sequence of the light chain variable domain present in the first antigen-binding site.

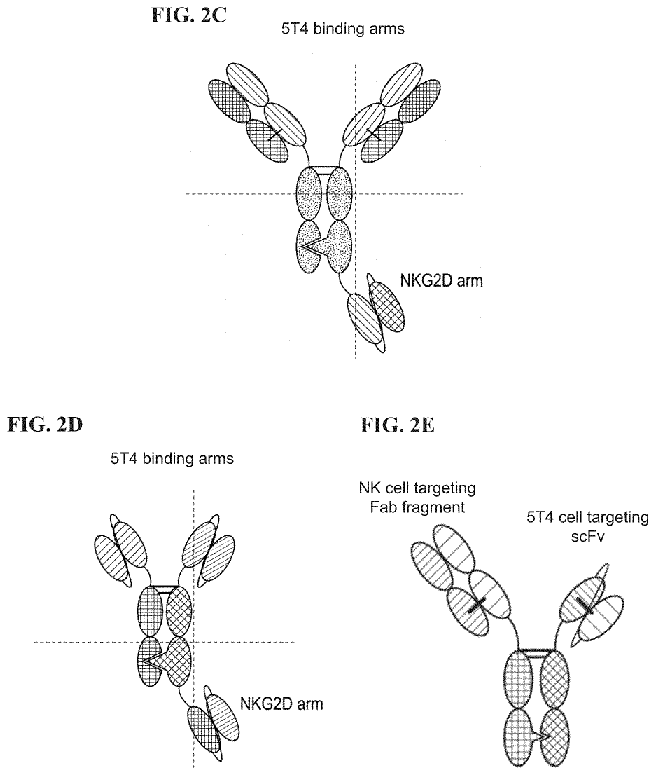

Another exemplary format involves a heterodimeric, multispecific antibody including a first immunoglobulin heavy chain, a second immunoglobulin heavy chain and an immunoglobulin light chain (e.g., FIG. 2A). In some embodiments, the first immunoglobulin heavy chain includes a first Fc (hinge-CH2-CH3) domain fused via either a linker or an antibody hinge to a single-chain variable fragment (scFv) composed of a heavy chain variable domain and light chain variable domain which pair and bind NKG2D, or bind 5T4. In some embodiments, the second immunoglobulin heavy chain includes a second Fc (hinge-CH2-CH3) domain, a second heavy chain variable domain and a CH1 heavy chain domain. The immunoglobulin light chain includes a light chain variable domain and a light chain constant domain. In some embodiments, the second immunoglobulin heavy chain pairs with the immunoglobulin light chain and binds to NKG2D or binds 5T4 with the proviso that when the first Fc domain is fused to an scFv that binds NKG2D, the second immunoglobulin heavy chain paired with the immunoglobulin light chain binds 5T4 but not NKG2D, and vice versa. In some embodiments, the scFv in the first immunoglobulin heavy chain binds 5T4; and the heavy chain variable domain in the second immunoglobulin heavy chain and the light chain variable domain in the immunoglobulin light chain, when paired, bind NKG2D (e.g., FIG. 2E). In some embodiments, the scFv in the first immunoglobulin heavy chain binds NKG2D; and the heavy chain variable domain in the second immunoglobulin heavy chain and the light chain variable domain in the immunoglobulin light chain, when paired, bind 5T4. In some embodiments, the first Fc domain and the second Fc domain together are able to bind to CD16 (e.g., FIG. 2A). In some embodiments, the first Fc domain and the second Fc domain together are able to bind to CD16 (e.g., FIG. 2A).

Another exemplary format involves a heterodimeric, multispecific antibody including a first immunoglobulin heavy chain, and a second immunoglobulin heavy chain (e.g., FIG. 2B). In some embodiments, the first immunoglobulin heavy chain includes a first Fc (hinge-CH2-CH3) domain fused via either a linker or an antibody hinge to a single-chain variable fragment (scFv) composed of a heavy chain variable domain and light chain variable domain, which pair and bind NKG2D, or bind 5T4. In some embodiments, the second immunoglobulin heavy chain includes a second Fc (hinge-CH2-CH3) domain fused via either a linker or an antibody hinge to a single-chain variable fragment (scFv) composed of a heavy chain variable domain and light chain variable domain which pair and bind NKG2D or bind 5T4, with the proviso that when the first Fc domain is fused to an scFv that binds NKG2D, the second Fc domain fused to an scFv binds 5T4, but not NKG2D, and vice versa. In some embodiments, the first Fc domain and the second Fc domain together are able to bind to CD16 (e.g., FIG. 2B).

In some embodiments, the single-chain variable fragment (scFv) described above is linked to the antibody constant domain via a hinge sequence. In some embodiments, the hinge comprises amino acids Ala-Ser or Gly-Ser. In some embodiments, the hinge comprises amino acids Ala-Ser. In some embodiments, the hinge comprises amino acids Ala-Ser. In some embodiments, the hinge connecting an scFv (e.g., an scFv that binds NKG2D or an scFv that binds 5T4) and the antibody heavy chain constant domain comprises amino acids Ala-Ser. In some embodiments, the hinge connecting an scFv (e.g., an scFv that binds NKG2D or an scFv that binds 5T4) and the antibody heavy chain constant domain comprises amino acids Gly-Ser. In some other embodiments, the hinge comprises amino acids Ala-Ser and Thr-Lys-Gly. The hinge sequence can provide flexibility of binding to the target antigen, and balance between flexibility and optimal geometry.

In some embodiments, the single-chain variable fragment (scFv) described above includes a heavy chain variable domain and a light chain variable domain. In some embodiments, the heavy chain variable domain forms a disulfide bridge (a.k.a., disulfide bond) with the light chain variable domain to enhance stability of the scFv. For example, a disulfide bridge can be formed between the C44 residue of the heavy chain variable domain and the C100 residue of the light chain variable domain, the amino acid positions numbered under the Kabat numbering scheme. In some embodiments, the heavy chain variable domain is linked to the light chain variable domain via a flexible linker. Any suitable linker can be used, for example, the (G₄S)₄ linker ((GlyGlyGlyGlySer)₄ (SEQ ID NO:119)). In some embodiments of the scFv, the heavy chain variable domain is positioned at the N-terminus of the light chain variable domain. In some embodiments of the scFv, the heavy chain variable domain is positioned at the C terminus of the light chain variable domain. In some embodiments, within an scFv comprising the VH and the VL of an anti-NKG2D antibody, the VH is positioned at the N-terminus of the VL. In some embodiments, within each scFv comprising the VH and the VL of an anti-5T4 antibody, the VH is positioned at the N-terminus of the VL.

The multispecific binding proteins described herein can further include one or more additional antigen-binding sites. The additional antigen-binding site(s) may be fused to the N-terminus of the constant region CH2 domain or to the C-terminus of the constant region CH3 domain, optionally via a linker sequence. In certain embodiments, the additional antigen-binding site(s) takes the form of a single-chain variable region (scFv) that is optionally disulfide-stabilized, resulting in a tetravalent or trivalent multispecific binding protein. For example, a multispecific binding protein includes a first antigen-binding site that binds NKG2D, a second antigen-binding site that binds 5T4, an additional antigen-binding site that binds 5T4, and an antibody constant region or a portion thereof sufficient to bind CD16 or a fourth antigen-binding site that binds CD16. Any one of these antigen binding sites can either take the form of a Fab fragment or an scFv, such as an scFv described above.

In some embodiments, the additional antigen-binding site binds a different epitope of 5T4 from the second antigen-binding site. In some embodiments, the additional antigen-binding site binds the same epitope as the second antigen-binding site. In some embodiments, the additional antigen-binding site comprises the same heavy chain and light chain CDR sequences as the second antigen-binding site. In some embodiments, the additional antigen-binding site comprises the same heavy chain and light chain variable domain sequences as the second antigen-binding site. In some embodiments, the additional antigen-binding site has the same amino acid sequence(s) as the second antigen-binding site. In some embodiments, the additional antigen-binding site comprises heavy chain and light chain variable domain sequences that are different from the heavy chain and light chain variable domain sequences of the second antigen-binding site. In some embodiments, the additional antigen-binding site has an amino acid sequence that is different from the sequence of the second antigen-binding site. In some embodiments, the second antigen-binding site and the additional antigen-binding site bind different tumor-associated antigens. In some embodiments, the second antigen-binding site and the additional antigen-binding site binds different antigens. Exemplary formats are shown in FIG. 2C and FIG. 2D. Accordingly, the multispecific binding proteins can provide bivalent engagement of 5T4. Bivalent engagement of 5T4 by the multispecific binding proteins can stabilize 5T4 on the tumor cell surface and enhance cytotoxicity of NK cells towards the tumor cells. Bivalent engagement of 5T4 by the multispecific binding proteins can confer stronger binding of the multispecific binding proteins to the tumor cells, thereby facilitating stronger cytotoxic response of NK cells towards the tumor cells, especially towards tumor cells expressing a low level of 5T4.

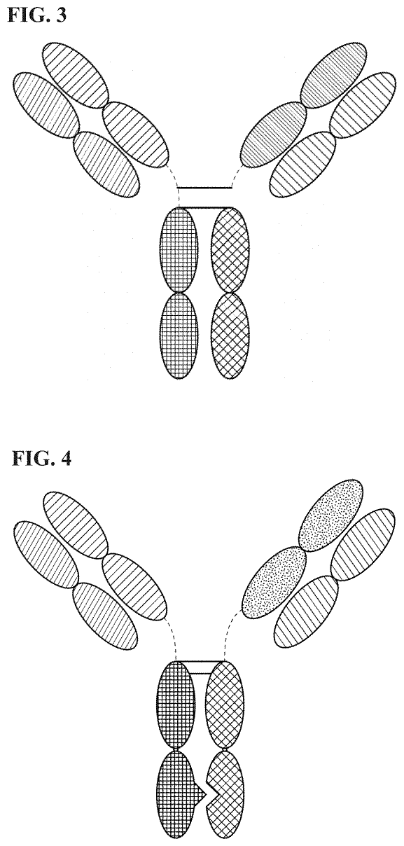

The multispecific binding proteins can take additional formats. In some embodiments, the multispecific binding protein is in the Triomab form (FIG. 3), which is a trifunctional, bispecific antibody that maintains an IgG-like shape. This chimera consists of two half antibodies, each with one light and one heavy chain, that originate from two parental antibodies.

In some embodiments, the multispecific binding protein is in a KiH Common Light Chain (LC) form, which incorporates the knobs-into-holes (KiH) technology (e.g., the multispecific binding protein represented in FIG. 4). The KiH Common LC form is a heterodimer comprising a Fab which binds to a first target, a Fab which binds to a second target, and an Fc domain stabilized by heterodimerization mutations. The two Fabs each comprise a heavy chain and light chain, wherein the heavy chain of each Fab differs from the other, and the light chain that pairs with each respective heavy chain is common to both Fabs.

In some embodiments, the multispecific binding protein is the KiH form, which involves the knobs-into-holes (KiHs) technology. The KiH involves engineering C_{H}3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structured-guided phage library screening (Atwell S, Ridgway J B, Wells J A, Carter P., Stable heterodimers from remodeling the domain interface of a homodimer using a phage display library, J. Mol. Biol. (1997) 270(1):26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426(9):1947-57; Mimoto F, Kadono S, Katada H, Igawa T, Kamikawa T, Hattori K. Crystal structure of a novel asymmetrically engineered Fc variant with improved affinity for FcTRs. Mol. Immunol. (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

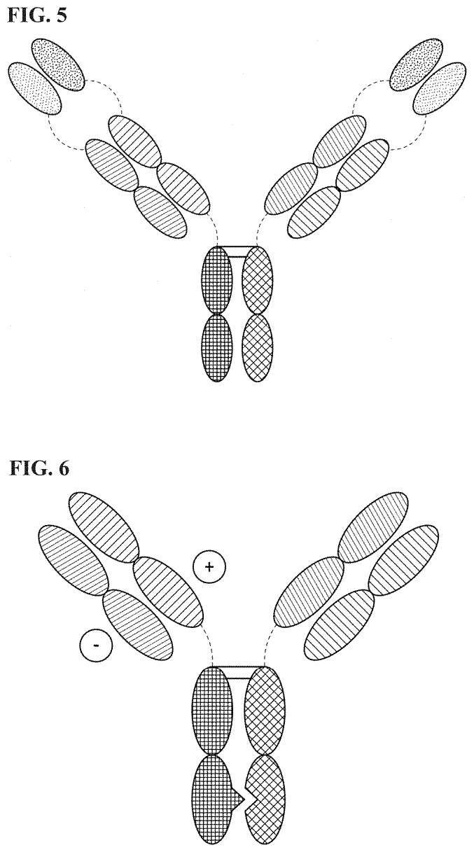

In some embodiments, the multispecific binding protein is in the dual-variable domain immunoglobulin (DVD-Ig™) form (FIG. 5), which combines the target binding domains of two monoclonal antibodies via flexible naturally occurring linkers and yields a tetravalent IgG-like molecule.

In some embodiments, the multispecific binding protein is in the Orthogonal Fab interface (Ortho-Fab) form (FIG. 6). In the ortho-Fab IgG approach (Lewis S M, Wu X, Pustilnik A, Sereno A, Huang F, Rick H L, et al., Generation of bispecific IgG antibodies by structure-based design of an orthogonal Fab interface. Nat. Biotechnol. (2014) 32(2):191-8), structure-based regional design introduces complementary mutations at the LC and HC_{VH-CH1} interface in only one Fab fragment, without any changes being made to the other Fab fragment.

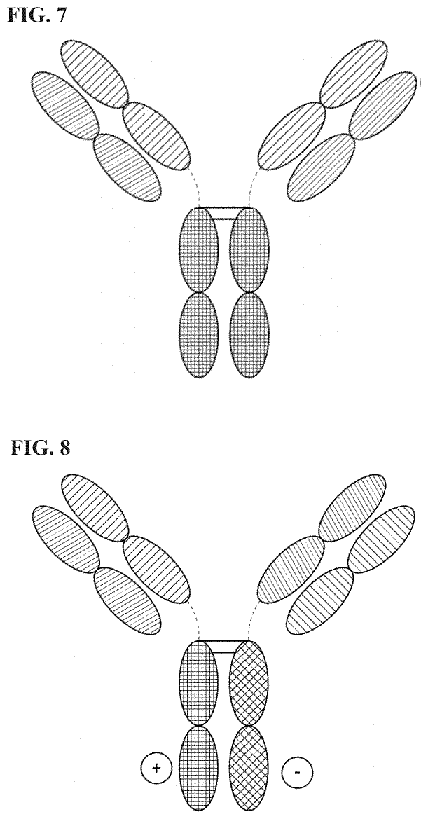

In some embodiments, the multispecific binding protein is in the 2-in-1 Ig format (FIG. 7). In some embodiments, the multispecific binding protein is in the ES form (FIG. 8), which is a heterodimeric construct containing two different Fab fragments binding to targets 1 and target 2 fused to the Fc. Heterodimerization is ensured by electrostatic steering mutations in the Fc.

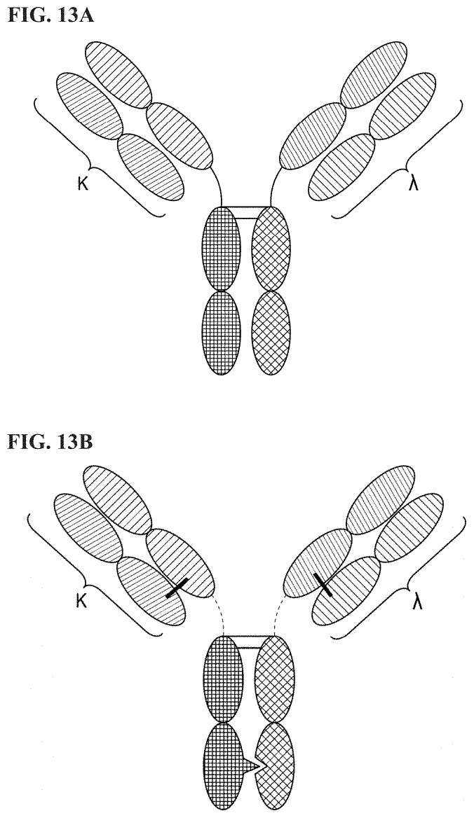

In some embodiments, the multispecific binding protein is in the Kλ-Body form, which is a heterodimeric construct with two different Fab fragments fused to Fc stabilized by heterodimerization mutations: Fab fragment 1 targeting antigen 1 contains kappa LC, and Fab fragment 2 targeting antigen 2 contains lambda LC. FIG. 13A is an exemplary representation of one form of a Kλ-Body; FIG. 13B is an exemplary representation of another Kλ-Body.

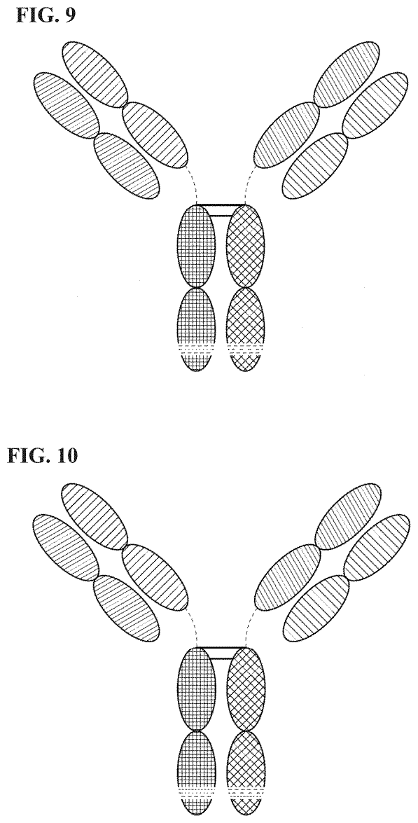

In some embodiments, the multispecific binding protein is in Fab Arm Exchange form (FIG. 9), which exchange Fab fragment arms by swapping a heavy chain and attached light chain (half-molecule) with a heavy-light chain pair from another molecule, which results in bispecific antibodies.

In some embodiments, the multispecific binding protein is in the SEED Body form (FIG. 10). The strand-exchange engineered domain (SEED) platform was designed to generate asymmetric and bispecific antibody-like molecules, a capability that expands therapeutic applications of natural antibodies. This protein engineering platform is based on exchanging structurally related sequences of immunoglobulin within the conserved CH3 domains. The SEED design allows efficient generation of AG/GA heterodimers, whereas disfavoring homodimerization of AG and GA SEED CH3 domains. (Muda M. et al., Protein Eng. Des. Sel. (2011, 24(5):447-54)).

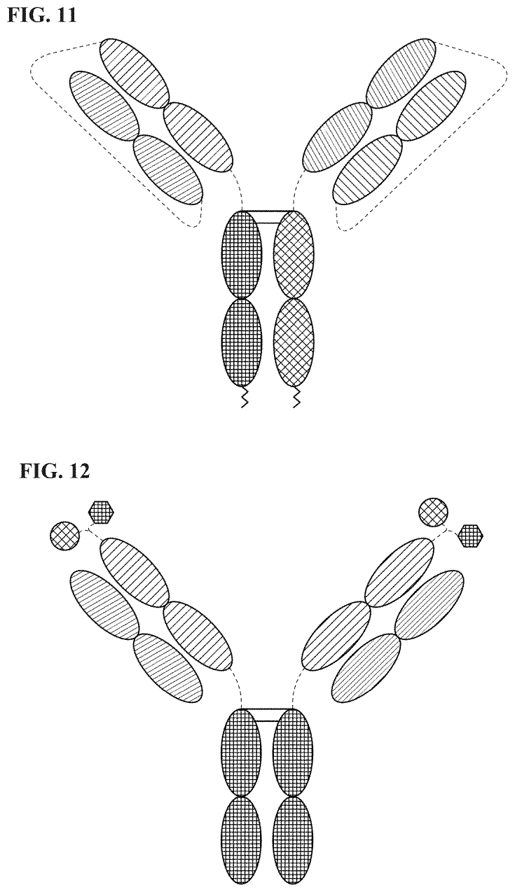

In some embodiments, the multispecific binding protein is in the LuZ-Y form (FIG. 11), in which a leucine zipper is used to induce heterodimerization of two different HCs. (Wranik, B J. et al., J. Biol. Chem. (2012), 287:43331-9).

In some embodiments, the multispecific binding protein is in the Cov-X-Body form (FIG. 12). In bispecific CovX-Bodies, two different peptides are joined together using a branched azetidinone linker and fused to the scaffold antibody under mild conditions in a site-specific manner. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V R et al., PNAS (2010), 107(52); 22611-22616).

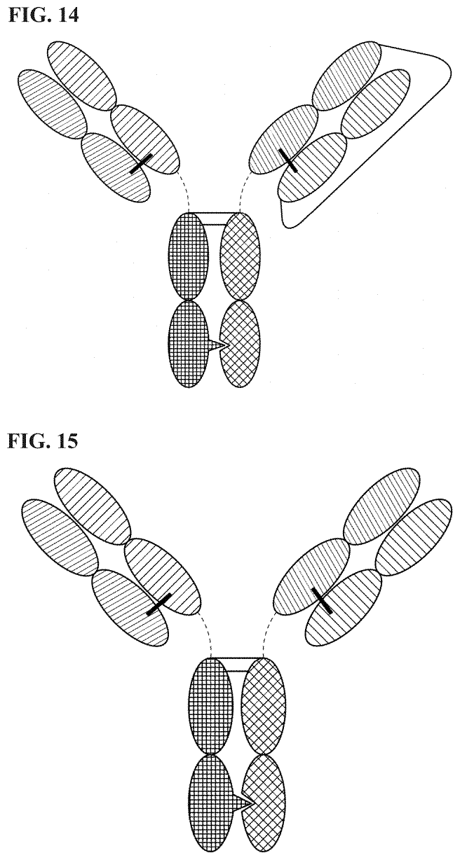

In some embodiments, the multispecific binding protein is in an OAsc-Fab heterodimeric form (FIG. 14) that includes Fab fragment binding to target 1, and scFab binding to target 2 fused to Fc. Heterodimerization is ensured by mutations in the Fc.

In some embodiments, the multispecific binding protein is in a DuetMab form (FIG. 15), which is a heterodimeric construct containing two different Fab fragments binding to antigens 1 and 2, and Fc stabilized by heterodimerization mutations. Fab fragments 1 and 2 contain differential S-S bridges that ensure correct LC and HC pairing.

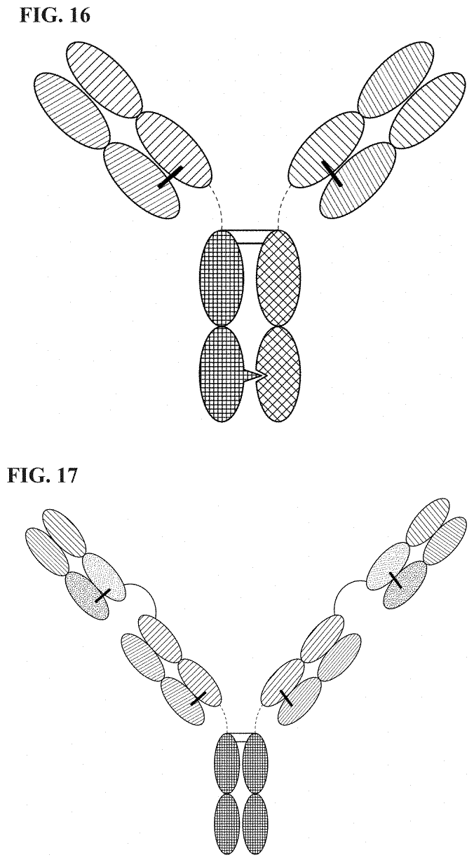

In some embodiments, the multispecific binding protein is in a CrossmAb form (FIG. 16), which is a heterodimeric construct with two different Fab fragments binding to targets 1 and 2, fused to Fc stabilized by heterodimerization. CL and CH1 domains and VH and VL domains are switched, e.g., CH1 is fused in-frame with VL, and CL is fused in-frame with VH.

In some embodiments, the multispecific binding protein is in a Fit-Ig form (FIG. 17), which is a homodimeric construct where Fab fragment binding to antigen 2 is fused to the N terminus of HC of Fab fragment that binds to antigen 1. The construct contains wild-type Fc.

In some embodiments, the multispecific binding protein provided herein may be in a form well known in the art, including, but not limited to a Fab fragment, a Fab′ fragment, F(ab′)₂ fragment, an Fv, a bispecific antibody, a bispecific Fab2, a bispecific (mab)₂, a humanized antibody, bispecific T-cell engager, bispecific NK cell engager, a single chain antibody (e.g., single-chain variable fragment or scFv), triomab, knobs-into-holes (kih) IgG with common light chain, crossmab, ortho-Fab IgG, DVD-Ig, 2 in 1-IgG, IgG-scFv, sdFv2-Fc, bi-nanobody, tandAb, dual-affinity retargeting antibody (DART), DART-Fc, scFv-HSA-scFv (where HSA=human serum albumin), or dock-and-lock (DNL)-Fab3.

Individual components of the multispecific binding proteins are described in more detail below.

### NKG2D-Binding Site or NKG2D-Binding Domain

Upon binding to the NKG2D receptor and CD16 receptor on natural killer cells, and 5T4, the multispecific binding proteins can engage more than one kind of NK-activating receptor, and may block the binding of natural ligands to NKG2D. In certain embodiments, the proteins can agonize NK cells in humans. In some embodiments, the proteins can agonize NK cells in humans and in other species such as rodents and cynomolgus monkeys. In some embodiments, the proteins can agonize NK cells in humans and in other species such as cynomolgus monkeys.

Table 1 lists polypeptide sequences of heavy chain variable domains and light chain variable domains that, in combination, can bind to NKG2D. In some embodiments, the heavy chain variable domain and the light chain variable domain are arranged in Fab format. In some embodiments, the heavy chain variable domain and the light chain variable domain are fused together to form an scFv.

The NKG2D binding sites or NKG2D binding domains listed in Table 1 can vary in their binding affinity to NKG2D, nevertheless, they all activate human NK cells.

Unless indicated otherwise, the CDR sequences provided in Table 1 are determined under Kabat numbering scheme. Table 1A provides CDR sequences according to Kabat numbering scheme. Table 1B provides CDR sequences according to Chothia numbering scheme. Table 1C provides CDR sequences according to IMGT numbering scheme. Table 1D provides CDR sequences according to Honegger numbering scheme.

TABLE 1
Sequences of Exemplary Antigen-Binding Sites or Antigen-Binding
Domains that Bind NKG2D

| | Heavy chain variable region | Light chain variable region |
|---|---|---|
| Clones | amino acid sequence | amino acid sequence |
| ADI- | QVQLQQWGAGLLKPSETLSLTCAVYG | DIQMTQSPSTLSASVGDRVTITCRAS |
| 27705 | GSFSGYYWSWIRQPPGKGLEWIGEID | QS1SSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYNSYPITFGGGTKV |
| | DPWGQGTLVTVSS | EIK |
| | (SEQ ID NO: 1) | (SEQ ID NO: 5) |
| | CDR1 (SEQ ID NO: 2) - | |
| | GSFSGYYWS (non-Kabat) | |
| | CDR2 (SEQ ID NO: 3) - | |
| | EIDHSGSTNYNPSLKS | |
| | CDR3 (SEQ ID NO: 4) - | |
| | ARARGPWSFDP (IMGT) | |
| ADI- | QVQLQQWGAGLLKPSETLSLTCAVYG | EIVLTQSPGTLSLSPGERATLSCRAS |
| 27724 | GSFSGYYWSWIRQPPGKGLEWIGEID | QSVSSSYLAWYQQKPGQAPRLLIYGA |
| | HSGSTNYNPSLKSRVTISVDTSKNQF | SSRATGIPDRFSGSGSGTDFTLTISR |
| | SLKLSSVTAADTAVYYCARARGPWSF | LEPEDFAVYYCQQYGSSPITFGGGTK |
| | DPWGQGTLVTVSS | VEIK |
| | (SEQ ID NO: 1) | (SEQ ID NO: 6) |
| ADI- | QVQLQQWGAGLLKPSETLSLTCAVYG | DIQMTQSPSTLSASVGDRVTITCRAS |
| 27740 | GSFSGYYWSWIRQPPGKGLEWIGEID | QSIGSWLAWYQQKPGKAPKLLIYKAS |
| (A40) | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYHSFYTFGGGTKVE |
| | DPWGQGTLVTVSS | IK |
| | (SEQ ID NO: 1) | (SEQ ID NO: 7) |
| ADI- | QVQLQQWGAGLLKPSETLSLTCAVYG | DIQMTQSPSTLSASVGDRVTITCRAS |
| 27741 | GSFSGYYWSWIRQPPGKGLEWIGEID | QSIGSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQSNSYYTFGGGTKVE |
| | DPWGQGTLVTVSS | IK |
| | (SEQ ID NO: 1) | (SEQ ID NO: 8) |
| ADI- | QVQLQQWGAGLLKPSETLSLTCAVYG | DIQMTQSPSTLSASVGDRVTITCRAS |
| 27743 | GSFSGYYWSWIRQPPGKGLEWIGEID | QSISSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYNSYPTFGGGTKVE |
| | DPWGQGTLVTVSS | IK |
| | (SEQ ID NO: 1) | (SEQ ID NO: 9) |
| ADI- | QVQLQQWGAGLLKPSETLSLTCAVYG | ELQMTQSPSSLSASVGDRVTITCRTS |
| 28153 | GSFSGYYWSWIRQPPGKGLEWIGEID | QSISSYLNWYQQKPGQPPKLLIYWAS |
| | HSGSTNYNPSLKSRVTISVDTSKNQF | TRESGVPDRFSGSGSGTDFTLTISSL |
| | SLKLSSVTAADTAVYYCARARGPWGF | QPEDSATYYCQQSYDIPYTFGQGTKL |
| | DPWGQGTLVTVSS | EIK |
| | (SEQ ID NO: 10) | (SEQ ID NO: 11) |
| ADI- | QVQLQQWGAGLLKPSETLSLTCAVYG | DIQMTQSPSTLSASVGDRVTITCRAS |
| 28226 | GSFSGYYWSWIRQPPGKGLEWIGEID | QSISSWLAWYQQKPGKAPKLLIYKAS |
| (C26) | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYGSFPITFGGGTKV |
| | DPWGQGTLVTVSS | EIK |
| | (SEQ ID NO: 1) | (SEQ ID NO: 12) |
| ADI- | QVQLQQWGAGLLKPSETLSLTCAVYG | DIQMTQSPSTLSASVGDRVTITCRAS |
| 28154 | GSFSGYYWSWIRQPPGKGLEWIGEID | QSISSWLAWYQQKPGKAPKLLIYKAS |
| | HSGSTNYNPSLKSRVTISVDTSKNQF | SLESGVPSRFSGSGSGTDFTLTISSL |
| | SLKLSSVTAADTAVYYCARARGPWSF | QPDDFATYYCQQSKEVPWTFGQGTKV |
| | DPWGQGTLVTVSS | EIK |
| | (SEQ ID NO: 1) | (SEQ ID NO: 13) |
| ADI- | QVQLQQWGAGLLKPSETLSLTCAVYG | DIQMTQSPSTLSASVGDRVTITCRAS |
| 29399 | GSFSGYYWSWIRQPPGKGLEWIGEID | QSISSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYNSFPTFGGGTKVE |
| | DPWGQGTLVTVSS | IK |
| | (SEQ ID NO: 1) | (SEQ ID NO: 14) |
| ADI- | QVQLQQWGAGLLKPSETLSLTCAVYG | DIQMTQSPSTLSASVGDRVTITCRAS |
| 29401 | GSFSGYYWSWIRQPPGKGLEWIGEID | QSIGSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYDIYPTFGGGTKVE |
| | DPWGQGTLVTVSS | IK |
| | (SEQ ID NO: 1) | (SEQ ID NO: 15) |
| ADI- | QVQLQQWGAGLLKPSETLSLTCAVYG | DIQMTQSPSTLSASVGDRVTITCRAS |
| 29403 | GSFSGYYWSWIRQPPGKGLEWIGEID | QSISSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYDSYPTFGGGTKVE |
| | DPWGQGTLVTVSS | IK |
| | (SEQ ID NO: 1) | (SEQ ID NO: 16) |
| ADI- | QVQLQQWGAGLLKPSETLSLTCAVYG | DIQMTQSPSTLSASVGDRVTITCRAS |
| 29405 | GSFSGYYWSWIRQPPGKGLEWIGEID | QSISSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYGSFPTFGGGTKVE |
| | DPWGQGTLVTVSS | IK |
| | (SEQ ID NO: 1) | (SEQ ID NO: 17) |
| ADI- | QVQLQQWGAGLLKPSETLSLTCAVYG | DIQMTQSPSTLSASVGDRVTITCRAS |
| 29407 | GSFSGYYWSWIRQPPGKGLEWIGEID | QSISSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYQSFPTFGGGTKVE |
| | DPWGQGTLVTVSS | IK |
| | (SEQ ID NO: 1) | (SEQ ID NO: 18) |
| ADI- | QVQLQQWGAGLLKPSETLSLTCAVYG | DIQMTQSPSTLSASVGDRVTITCRAS |
| 29419 | GSFSGYYWSWIRQPPGKGLEWIGEID | QSISSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYSSFSTFGGGTKVE |
| | DPWGQGTLVTVSS | IK |
| | (SEQ ID NO: 1) | (SEQ ID NO: 19) |
| ADI- | QVQLQQWGAGLLKPSETLSLTCAVYG | DIQMTQSPSTLSASVGDRVTITCRAS |
| 29421 | GSFSGYYWSWIRQPPGKGLEWIGEID | QSISSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYESYSTFGGGTKVE |
| | DPWGQGTLVTVSS | IK |
| | (SEQ ID NO: 1) | (SEQ ID NO: 20) |
| ADI- | QVQLQQWGAGLLKPSETLSLTCAVYG | DIQMTQSPSTLSASVGDRVTITCRAS |
| 29424 | GSFSGYYWSWIRQPPGKGLEWIGEID | QSISSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYDSFITFGGGTKVE |
| | DPWGQGTLVTVSS | IK |
| | (SEQ ID NO: 1) | (SEQ ID NO: 21) |
| ADI- | QVQLQQWGAGLLKPSETLSLTCAVYG | DIQMTQSPSTLSASVGDRVTITCRAS |
| 29425 | GSFSGYYWSWIRQPPGKGLEWIGEID | QSISSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYQSYPTFGGGTKVE |
| | DPWGQGTLVTVSS | IK |
| | (SEQ ID NO: 1) | (SEQ ID NO: 22) |
| ADI- | QVQLQQWGAGLLKPSETLSLTCAVYG | DIQMTQSPSTLSASVGDRVTITCRAS |
| 29426 | GSFSGYYWSWIRQPPGKGLEWIGEID | QSIGSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYHSFPTFGGGTKVE |
| | DPWGQGTLVTVSS | IK |
| | (SEQ ID NO: 1) | (SEQ ID NO: 23) |
| ADI- | QVQLQQWGAGLLKPSETLSLTCAVYG | DIQMTQSPSTLSASVGDRVTITCRAS |
| 29429 | GSFSGYYWSWIRQPPGKGLEWIGEID | QSIGSWLAWYQQKPGKAPKLLIYKAS |
| | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYELYSYTFGGGTKV |
| | DPWGQGTLVTVSS | EIK |
| | (SEQ ID NO: 1) | (SEQ ID NO: 24) |
| ADI- | QVQLQQWGAGLLKPSETLSLTCAVYG | DIQMTQSPSTLSASVGDRVTITCRAS |
| 29447 | GSFSGYYWSWIRQPPGKGLEWIGEID | QSISSWLAWYQQKPGKAPKLLIYKAS |
| (F47) | HSGSTNYNPSLKSRVTISVDTSKNQF | SLESGVPSRFSGSGSGTEFTLTISSL |
| | SLKLSSVTAADTAVYYCARARGPWSF | QPDDFATYYCQQYDTFITFGGGTKVE |
| | DPWGQGTLVTVSS | IK |
| | (SEQ ID NO: 1) | (SEQ ID NO: 25) |
| ADI- | QVQLVQSGAEVKKPGSSVKVSCKASG | DIVMTQSPDSLAVSLGERATINCKSS |
| 27727 | GTFSSYAISWVRQAPGQGLEWMGGII | QSVLYSSNNKNYLAWYQQKPGQPPKL |
| | PIFGTANYAQKFQGRVTITADESTST | LIYWASTRESGVPDRFSGSGSGTDFT |
| | AYMELSSLRSEDTAVYYCARGDSSIR | LTISSLQAEDVAVYYCQQYYSTPITF |
| | HAYYYYGMDVWGQGTTVTVSS | GGGTKVEIK |
| | (SEQ ID NO: 26) | (SEQ ID NO: 32) |
| | CDR1- GTFSSYAIS (non- | CDR1 (SEQ ID NO: 33) - |
| | Kabat) (SEQ ID NO: 27) or | KSSQSVLYSSNNKNYLA |
| | SYAIS (SEQ ID NO: 28) | CDR2 (SEQ ID NO: 34) - |
| | CDR2 (SEQ ID NO: 29) - | WASTRES |
| | GIIPIFGTANYAQKFQG | CDR3 (SEQ ID NO: 35) - |
| | CDR3 - ARGDSSIRHAYYYYGMDV | QQYYSTPIT |
| | (non-Kabat) (SEQ ID NO: 30) | |
| | or GDSSIRHAYYYYGMDV (SEQ | |
| | ID NO: 31) | |
| ADI- | QLQLQESGPGLVKPSETLSLTCTVSG | EIVLTQSPATLSLSPGERATLSCRAS |
| 29443 | GSISSSSYYWGWIRQPPGKGLEWIGS | QSVSRYLAWYQQKPGQAPRLLIYDAS |
| (F43) | IYYSGSTYYNPSLKSRVTISVDTSKN | NRATGIPARFSGSGSGTDFTLTISSL |
| | QFSLKLSSVTAADTAVYYCARGSDRF | EPEDFAVYYCQQFDTWPPTFGGGTKV |
| | HPYFDYWGQGTLVTVSS | EIK |
| | (SEQ ID NO: 36) | (SEQ ID NO: 42) |
| | CDR1- GSISSSSYYWG (non- | CDR1 (SEQ ID NO: 43) - |
| | Kabat) (SEQ ID NO: 37) or | RASQSVSRYLA |
| | SSSYYWG (SEQ ID NO: 38) | CDR2 (SEQ ID NO: 44) - |
| | CDR2 (SEQ ID NO: 39) - | DASNRAT |
| | SIYYSGSTYYNPSLKS | CDR3 (SEQ ID NO: 45) - |
| | CDR3 - ARGSDRFHPYFDY (non- | QQFDTWPPT |
| | Kabat) (SEQ ID NO: 40) or | |
| | GSDRFHPYFDY (SEQ ID NO: 41) | |
| ADI- | QVQLQQWGAGLLKPSETLSLTCAVYG | DIQMTQSPSTLSASVGDRVTITCRAS |
| 29404 | GSFSGYYWSWIRQPPGKGLEWIGEID | QSISSWLAWYQQKPGKAPKLLIYKAS |
| (F04) | HSGSTNYNPSLKSRVTISVDTSKNQF | SLESGVPSRFSGSGSGTEFTLTISSL |
| | SLKLSSVTAADTAVYYCARARGPWSF | QPDDFATYYCEQYDSYPTFGGGTKVE |
| | DPWGQGTLVTVSS (SEQ ID | IK |
| | NO: 1) | (SEQ ID NO: 46) |
| ADI- | QVQLVQSGAEVKKPGSSVKVSCKASG | DIVMTQSPDSLAVSLGERATINCESS |
| 28200 | GTFSSYAISWVRQAPGQGLEWMGGII | QSLLNSGNQKNYLTWYQQKPGQPPKP |
| | PIFGTANYAQKFQGRVTITADESTST | LIYWASTRESGVPDRFSGSGSGTDFT |
| | AYMELSSLRSEDTAVYYCARRGRKAS | LTISSLQAEDVAVYYCQNDYSYPYTF |
| | GSFYYYYGMDVWGQGTTVTVSS | GQGTKLEIK |
| | (SEQ ID NO: 47) | (SEQ ID NO: 49) |
| | CDR1 (SEQ ID NO: 27) - | CDR1 (SEQ ID NO: 50) - |
| | GTFSSYAIS (non-Kabat) | ESSQSLLNSGNQKNYLT |
| | CDR2 (SEQ ID NO: 29) - | CDR2 (SEQ ID NO: 34) - |
| | G11PIFGTANYAQKFQG | WASTRES |
| | CDR3 (SEQ ID NO: 48) - | CDR3 (SEQ ID NO: 51) - |
| | ARRGRKASGSFYYYYGMDV (IMGT) | QNDYSYPYT |
| ADI- | QVQLVQSGAEVKKPGASVKVSCKASG | EIVMTQSPATLSVSPGERATLSCRAS |
| 29379 | YTFTSYYMHWVRQAPGQGLEWMGIIN | QSVSSNLAWYQQKPGQAPRLLIYGAS |
| (E79) | PSGGSTSYAQKFQGRVTMTRDTSTST | TRATGIPARFSGSGSGTEFTLTISSL |
| | VYMELSSLRSEDTAVYYCARGAPNYG | QSEDFAVYYCQQYDDWPFTFGGGTKV |
| | DTTHDYYYMDVWGKGTTVTVSS | EIK |
| | (SEQ ID NO: 52) | (SEQ ID NO: 58) |
| | CDR1 (SEQ ID NO: 53) - | CDR1 (SEQ ID NO: 59) - |
| | YTFTSYYMH (non-Kabat) or | RASQSVSSNLA |
| | SYYMH (SEQ ID NO: 54) | CDR2 (SEQ ID NO: 60) - |
| | CDR2 (SEQ ID NO: 55) - | GASTRAT |
| | IINPSGGSTSYAQKFQG | CDR3 (SEQ ID NO: 61) - |
| | CDR3 - ARGAPNYGDTTHDYYYMDV | QQYDDWPFT |
| | (non-Kabat) (SEQ ID NO: 56) | |
| | or GAPNYGDTTHDYYYMDV (SEQ | |
| | ID NO: 57) | |
| ADI- | QVQLVQSGAEVKKPGASVKVSCKASG | EIVLTQSPGTLSLSPGERATLSCRAS |
| 29463 | YTFTGYYMHWVRQAPGQGLEWMGWIN | QSVSSNLAWYQQKPGQAPRLLIYGAS |
| (F63) | PNSGGTNYAQKFQGRVTMTRDTSIST | TRATGIPARFSGSGSGTEFTLTISSL |
| | AYMELSRLRSDDTAVYYCARDTGEYY | QSEDFAVYYCQQDDYWPPTFGGGTKV |
| | DTDDHGMDVWGQGTTVTVSS | EIK |
| | (SEQ ID NO: 62) | (SEQ ID NO: 68) |
| | CDR1 - YTFTGYYMH (non- | CDRI (SEQ ID NO: 59) - |
| | Kabat) (SEQ ID NO: 63) or | RASQSVSSNLA |
| | GYYMH (SEQ ID NO: 64) | CDR2 (SEQ ID NO: 60) - |
| | CDR2 (SEQ ID NO: 65) - | GASTRAT |
| | WINPNSGGTNYAQKFQG | CDR3 (SEQ ID NO: 69) - |
| | CDR3 - ARDTGEYYDTDDHGMDV | QQDDYWPPT |
| | (non-Kabat) (SEQ ID NO: 66) | |
| | or DTGEYYDTDDHGMDV (SEQ ID | |
| | NO: 67) | |
| ADI- | EVQLLESGGGLVQPGGSLRLSCAASG | DIQMTQSPSSVSASVGDRVTITCRAS |
| 27744 | FTFSSYAMSWVRQAPGKGLEWVSAIS | QGIDSWLAWYQQKPGKAPKLLIYAAS |
| (A44) | GSGGSTYYADSVKGRFTISRDNSKNT | SLQSGVPSRFSGSGSGTDFTLTISSL |
| | LYLQMNSLRAEDTAVYYCAKDGGYYD | QPEDFATYYCQQGVSYPRTFGGGTKV |
| | SGAGDYWGQGTLVTVSS | EIK |
| | (SEQ ID NO: 70) | (SEQ ID NO: 75) |
| | CDR1- FTFSSYAMS (non- | CDR1 (SEQ ID NO: 76) - |
| | Kabat) (SEQ ID NO:1) or | RASQGIDSWLA |
| | SYAMS (SEQ ID NO: 115) | CDR2 (SEQ ID NO: 77) - |
| | CDR2 (SEQ ID NO: 72) - | AASSLQS |
| | AISGSGGSTYYADSVKG | CDR3 (SEQ ID NO: 78) - |
| | CDR3 - AKDGGYYDSGAGDY | QQGVSYPRT |
| | (non-Kabat) (SEQ ID NO: 73) | |
| | or DGGYYDSGAGDY (SEQ ID | |
| | NO: 74) | |
| ADI- | EVQLVESGGGLVKPGGSLRLSCAASG | DIQMTQSPSSVSASVGDRVTITCRAS |
| 27749 | FTFSSYSMNWVRQAPGKGLEWVSSIS | QGISSWLAWYQQKPGKAPKLLIYAAS |
| (A49) | SSSSYIYYADSVKGRFTISRDNAKNS | SLQSGVPSRFSGSGSGTDFTLTISSL |
| | LYLQMNSLRAEDTAVYYCARGAPMGA | QPEDFATYYCQQGVSFPRTFGGGTKV |
| | AAGWFDPWGQGTLVTVSS | EIK |
| | (SEQ ID NO: 79) | (SEQ ID NO: 85) |
| | CDR1 - FTFSSYSMN (SEQ ID | CDR1 (SEQ ID NO: 86) - |
| | NO: 80) (non-Kabat) or | RASQGISSWLA |
| | SYSMN (SEQ ID NO: 81) | CDR2 (SEQ ID NO: 77) - |
| | CDR2 (SEQ ID NO: 82) - | AASSLQS |
| | SISSSSSYIYYADSVKG | CDR3 (SEQ ID NO: 87) - |
| | CDR3- ARGAPMGAAAGWFDP (SEQ | QQGVSFPRT |
| | ID NO: 83) (non-Kabat) or | |
| | GAPMGAAAGWFDP (SEQ ID | |
| | NO: 84) | |
| | scFv (VL-VH) with Q44C in VH and G100C in VL, linker | |
| | italicized: | |
| | DIQMTQSPSSVSASVGDRVTITCRASQGISSWLAWYQQKPGKAPKLLIYAASSLQ | |
| | SGVPSRFSGSGSGTDFTLTISSLQPEDFATYYCQQGVSFPRTFGCGTKVEIKGGG | |
| | GSGGGGSGGGGSGGGGSEVQLVESGGGLVKPGGSLRLSCAASGFTFSSYSMNWVR | |
| | QAPGKCLEWVSSISSSSSYIYYADSVKGRFTISRDNAKNSLYLQMNSLRAEDTAV | |
| | YYCARGAPMGAAAGWFDPWGQGTLVTVSS (SEQ ID NO: 88) | |
| ADI- | QVQLVQSGAEVKKPGASVKVSCKASG | EIVLTQSPATLSLSPGERATLSCRAS |
| 29378 | YTFTSYYMHWVRQAPGQGLEWMGIIN | QSVSSYLAWYQQKPGQAPRLL1YDAS |
| (E78) | PSGGSTSYAQKFQGRVTMTRDTSTST | NRATGIPARFSGSGSGTDFTLTISSL |
| | VYMELSSLRSEDTAVYYCAREGAGFA | EPEDFAVYYCQQSDNWPFTFGGGTKV |
| | YGMDYYYMDVWGKGTTVTVSS | EIK |
| | (SEQ ID NO: 89) | (SEQ ID NO: 92) |
| | CDR1 - YTFTSYYMH (SEQ ID | CDR1 (SEQ ID NO: 93) - |
| | NO: 53) (non-Kabat) or | RASQSVSSYLA |
| | SYYMH (SEQ ID NO: 54) | CDR2 (SEQ ID NO: 44) - |
| | CDR2 (SEQ ID NO: 55) - | DASNRAT |
| | IINPSGGSTSYAQKFQG | CDR3 (SEQ ID NO: 94) - |
| | CDR3 - AREGAGFAYGMDYYYMDV | QQSDNWPFT |
| | (SEQ ID NO: 90) (non-Kabat) | |
| | or EGAGFAYGMDYYYMDV (SEQ | |
| | ID NO: 91) | |
| A49MI | EVQLVESGGGLVKPGGSLRLSCAASG | DIQMTQSPSSVSASVGDRVTITCRAS |
| | FTFSSYSMNWVRQAPGKGLEWVSSIS | QGISSWLAWYQQKPGKAPKLLIYAAS |
| | SSSSYIYYADSVKGRFTISRDNAKNS | SLQSGVPSRFSGSGSGTDFTLTISSL |
| | LYLQMNSLRAEDTAVYYCARGAPIGA | QPEDFATYYCQQGVSFPRTFGGGTKV |
| | AAGWFDPWGQGTLVTVSS (SEQ ID | EIK |
| | NO: 95) | (SEQ ID NO: 85) |
| | CDR1: FTFSSYSMN (SEQ ID | CDR1 (SEQ ID NO: 86) - |
| | NO: 80) (non-Kabat) or | RASQGISSWLA |
| | SYSMN (SEQ ID NO: 81) | CDR2 (SEQ ID NO: 77) - |
| | CDR2: SISSSSSYIYYADSVKG | AASSLQS |
| | (SEQ ID NO: 82) | CDR3 (SEQ ID NO: 87) - |
| | CDR3: ARGAPIGAAAGWFDP (SEQ | QQGVSFPRT |
| | ID NO: 96) (non-Kabat) or | |
| | GAPIGAAAGWFDP | |
| | (SEQ ID NO: 97) | |
| | scFv (VL-VH) with Q44C in VH and G100C in VL, linker | |
| | italicized: | |
| | DIQMTQSPSSVSASVGDRVTITCRASQGISSWLAWYQQKPGKAPKLLIYAASSLQ | |
| | SGVPSRFSGSGSGTDFTLTISSLQPEDFATYYCQQGVSFPRTFGCGTKVEIKGGG | |
| | GSGGGGSGGGGSGGGGSEVQLVESGGGLVKPGGSLRLSCAASGFTFSSYSMNWVR | |
| | QAPGKCLEWVSSISSSSSYIYYADSVKGRFTISRDNAKNSLYLQMNSLRAEDTAV | |
| | YYCARGAPIGAAAGWFDPWGQGTLVTVSS (SEQ ID NO: 288) | |
| A49MQ | EVQLVESGGGLVKPGGSLRLSCAASG | DIQMTQSPSSVSASVGDRVTITCRAS |
| | FTFSSYSMNWVRQAPGKGLEWVSSIS | QGISSWLAWYQQKPGKAPKLLIYAAS |
| | SSSSYIYYADSVKGRFTISRDNAKNS | SLQSGVPSRFSGSGSGTDFTLTISSL |
| | LYLQMNSLRAEDTAVYYCARGAPQGA | QPEDFATYYCQQGVSFPRTFGGGTKV |
| | AAGWFDPWGQGTLVTVSS | EIK |
| | (SEQ ID NO: 98) | (SEQ ID NO: 85) |
| | CDR1: FTFSSYSMN (SEQ ID | CDR1 (SEQ ID NO: 86) - |
| | NO: 80) (non-Kabat) or | RASQGISSWLA |
| | SYSMN (SEQ ID NO: 81) | CDR2 (SEQ ID NO: 77) - |
| | CDR2: SISSSSSYIYYADSVKG | AASSLQS |
| | (SEQ ID NO: 82) | CDR3 (SEQ ID NO: 87) - |
| | CDR3 - ARGAPQGAAAGWFDP | QQGVSFPRT |
| | (SEQ ID NO: 99) (non-Kabat) | |
| | or GAPQGAAAGWFDP (SEQ ID | |
| | NO: 100) | |
| A49ML | EVQLVESGGGLVKPGGSLRLSCAASG | DIQMTQSPSSVSASVGDRVTITCRAS |
| | FTFSSYSMNWVRQAPGKGLEWVSSIS | QGISSWLAWYQQKPGKAPKLLIYAAS |
| | SSSSYIYYADSVKGRFTISRDNAKNS | SLQSGVPSRFSGSGSGTDFTLTISSL |
| | LYLQMNSLRAEDTAVYYCARGAPLGA | QPEDFATYYCQQGVSFPRTFGGGTKV |
| | AAGWFDPWGQGTLVTVSS | EIK |
| | (SEQ ID NO: 101) | (SEQ ID NO: 85) |
| | CDR1: FTFSSYSMN (SEQ ID | CDR1 (SEQ ID NO: 86) - |
| | NO: 80) (non-Kabat) or | RASQGISSWLA |
| | SYSMN (SEQ ID NO: 81) | CDR2 (SEQ ID NO: 77) - |
| | CDR2: SISSSSSYIYYADSVKG | AASSLQS |
| | (SEQ ID NO: 82) | CDR3 (SEQ ID NO: 87) - |
| | CDR3 - ARGAPLGAAAGWFDP | QQGVSFPRT |
| | (SEQ ID NO: 102) (non- | |
| | Kabat) or GAPLGAAAGWFDP | |
| | (SEQ ID NO: 103) | |
| A49MF | EVQLVESGGGLVKPGGSLRLSCAASG | DIQMTQSPSSVSASVGDRVTITCRAS |
| | FTFSSYSMNWVRQAPGKGLEWVSSIS | QGISSWLAWYQQKPGKAPKLLIYAAS |
| | SSSSYIYYADSVKGRFTISRDNAKNS | SLQSGVPSRFSGSGSGTDFTLTISSL |
| | LYLQMNSLRAEDTAVYYCARGAPFGA | QPEDFATYYCQQGVSFPRTFGGGTKV |
| | AAGWFDPWGQGTLVTVSS | EIK |
| | (SEQ ID NO: 104) | (SEQ ID NO: 85) |
| | CDR1: FTFSSYSMN (SEQ ID | CDR1 (SEQ ID NO: 86) - |
| | NO: 80) (non-Kabat) or | RASQGISSWLA |
| | SYSMN (SEQ ID NO: 81) | CDR2 (SEQ ID NO: 77) - |
| | CDR2: SISSSSSYIYYADSVKG | AASSLQS |
| | (SEQ ID NO: 82) | CDR3 (SEQ ID NO: 87) - |
| | CDR3 - ARGAPFGAAAGWEDP | QQGVSFPRT |
| | (SEQ ID NO: 105) (non- | |
| | Kabat) or GAPFGAAAGWFDP | |
| | (SEQ ID NO: 106) | |
| A49MV | EVQLVESGGGLVKPGGSLRLSCAASG | DIQMTQSPSSVSASVGDRVTITCRAS |
| | FTFSSYSMNWVRQAPGKGLEWVSSIS | QGISSWLAWYQQKPGKAPKLLIYAAS |
| | SSSSYIYYADSVKGRFTISRDNAKNS | SLQSGVPSRFSGSGSGTDFTLTISSL |
| | LYLQMNSLRAEDTAVYYCARGAPVGA | QPEDFATYYCQQGVSFPRTFGGGTKV |
| | AAGWFDPWGQGTLVTVSS | EIK |
| | (SEQ ID NO: 107) | (SEQ ID NO: 85) |
| | CDR1: FTFSSYSMN (SEQ ID | CDR1 (SEQ ID NO: 86) - |
| | NO: 80) (non-Kabat) or | RASQGISSWLA |
| | SYSMN (SEQ ID NO: 81) | CDR2 (SEQ ID NO: 77) - |
| | CDR2: SISSSSSYIYYADSVKG | AASSLQS |
| | (SEQ ID NO: 82) | CDR3 (SEQ ID NO: 87) - |
| | CDR3- ARGAPVGAAAGWFDP (SEQ | QQGVSFPRT |
| | ID NO: 108) (non-Kabat) or | |
| | GAPVGAAAGWFDP (SEQ ID | |
| | NO: 109) | |
| A49- | EVQLVESGGGLVKPGGSLRLSCAASG | DIQMTQSPSSVSASVGDRVTITCRAS |
| consensus | FTFSSYSMNWVRQAPGKGLEWVSSIS | QGISSWLAWYQQKPGKAPKLLIYAAS |
| | SSSSYIYYADSVKGRFTISRDNAKNS | SLQSGVPSRFSGSGSGTDFTLTISSL |
| | LYLQMNSLRAEDTAVYYCARGAPXGA | QPEDFATYYCQQGVSFPRTFGGGTKV |
| | AAGWFDPWGQGTLVTVSS, | EIK |
| | wherein X is M, L, I, V, | (SEQ ID NO: 85) |
| | Q, or F | CDR1 (SEQ ID NO: 86) - |
| | (SEQ ID NO: 110) | RASQGISSWLA |
| | CDR1: FTFSSYSMN (SEQ ID | CDR2 (SEQ ID NO: 77) - |
| | NO: 80) (non-Kabat) or | AASSLQS |
| | SYSMN (SEQ ID NO: 81) | CDR3 (SEQ ID NO: 87) - |
| | CDR2: SISSSSSYIYYADSVKG | QQGVSFPRT |
| | (SEQ ID NO: 82) | |
| | CDR3- ARGAPXGAAAGWFDP (SEQ | |
| | ID NO: 111) (non-Kabat) or | |
| | GAPXGAAAGWFDP (SEQ ID | |
| | NO: 112), wherein X is M, | |
| | L, I, V, Q, or F | |
| NKG2D | QVQLVESGGGLVKPGGSLRLSCAASG | QSALTQPASVSGSPGQSITISCSGSS |
| binder in | FTFSSYGMHWVRQAPGKGLEWVAFIR | SNIGNNAVNWYQQLPGKAPKLLIYYD |
| US | YDGSNKYYADSVKGRFTISRDNSKNT | DLLPSGVSDRFSGSKSGTSAFLAISG |
| 9,273,136 | LYLQMNSLRAEDTAVYYCAKDRGLGD | LQSEDEADYYCAAWDDSLNGPVFGGG |
| | GTYFDYWGQGTTVTVSS (SEQ ID | TKLTVL (SEQ ID NO: 114) |
| | NO: 113) | |
| NKG2D | QVHLQESGPGLVKPSETLSLTCTVSD | EIVLTQSPGTLSLSPGERATLSCRAS |
| binder in | DSISSYYWSWIRQPPGKGLEWIGHIS | QSVSSSYLAWYQQKPGQAPRLLIYGA |
| US | YSGSANYNPSLKSRVTISVDTSKNQF | SSRATGIPDRFSGSGSGTDFTLTISR |
| 7,879,985 | SLKLSSVTAADTAVYYCANWDDAFNI | LEPEDFAVYYCQQYGSSPWTFGQGTK |
| | WGQGTMVTVSS (SEQ ID | VEIK (SEQ ID NO: 117) |
| | NO: 116) | |

TABLE 1A
CDRs for illustrative anti-NKG2D binding antigen binding domains (Kabat)

| Ab | VH - | VH - | VH - | VL - | VL - | VL - |
|---|---|---|---|---|---|---|
| Name | CDR1 | CDR2 | CDR3 | CDR1 | CDR2 | CDR3 |
| 1 | GYYWS | EIDHSGSTNYNPSLKS | ARGPWSFDP | RASQSISSWLA | KASSLES | QQYNSYPIT |
| | SEQ ID | SEQ ID NO: 3 | SEQ ID | SEQ ID | SEQ ID | SEQ ID |
| | NO: 298 | | NO: 299 | NO: 300 | NO: 301 | NO: 302 |
| 2 | GYYWS | EIDHSGSTNYNPSLKS | ARGPWSFDP | RASQSVSSSYLA | GASSRAT | QQYGSSPIT |
| | SEQ ID | SEQ ID NO: 3 | SEQ ID | SEQ ID | SEQ ID | SEQ ID |
| | NO: 298 | | NO: 299 | NO: 303 | NO: 304 | NO: 305 |
| 3 | GYYWS | EIDHSGSTNYNPSLKS | ARGPWSFDP | RASQSIGSWLA | KASSLES | QQYHSFYT |
| | SEQ ID | SEQ ID NO: 3 | SEQ ID | SEQ ID | SEQ ID | SEQ ID |
| | NO: 298 | | NO: 299 | NO: 306 | NO: 301 | NO: 307 |
| 4 | GYYWS | EIDHSGSTNYNPSLKS | ARGPWSFDP | RASQSIGSWLA | KASSLES | QQSNSYYT |
| | SEQ ID | SEQ ID NO: 3 | SEQ ID | SEQ ID | SEQ ID | SEQ ID |
| | NO: 298 | | NO: 299 | NO: 306 | NO: 301 | NO: 308 |
| 5 | GYYWS | EIDHSGSTNYNPSLKS | ARGPWSFDP | RASQSISSWLA | KASSLES | QQYNSYPT |
| | SEQ ID | SEQ ID NO: 3 | SEQ ID | SEQ ID | SEQ ID | SEQ ID |
| | NO: 298 | | NO: 299 | NO: 300 | NO: 301 | NO: 309 |
| 6 | GYYWS | EIDHSGSTNYNPSLKS | ARGPWGFDP | RTSQSISSYLN | WASTRES | QQSYDIPYT |
| | SEQ ID | SEQ ID NO: 3 | SEQ ID | SEQ ID | SEQ ID | SEQ ID |
| | NO: 298 | | NO: 310 | NO: 311 | NO: 34 | NO: 312 |
| 7 | GYYWS | EIDHSGSTNYNPSLKS | ARGPWSFDP | RASQSISSWLA | KASSLES | QQYGSFPIT |
| | SEQ ID | SEQ ID NO: 3 | SEQ ID | SEQ ID | SEQ ID | SEQ ID |
| | NO: 298 | | NO: 299 | NO: 300 | NO: 301 | NO: 313 |
| 8 | GYYWS | EIDHSGSTNYNPSLKS | ARGPWSFDP | RASQSISSWLA | KASSLES | QQSKEVPWT |
| | SEQ ID | SEQ ID NO: 3 | SEQ ID | SEQ ID | SEQ ID | SEQ ID |
| | NO: 298 | | NO: 299 | NO: 300 | NO: 301 | NO: 314 |
| 9 | GYYWS | EIDHSGSTNYNPSLKS | ARGPWSFDP | RASQSISSWLA | KASSLES | QQYNSFPT |
| | SEQ ID | SEQ ID NO: 3 | SEQ ID | SEQ ID | SEQ ID | SEQ ID |
| | NO: 298 | | NO: 299 | NO: 300 | NO: 301 | NO: 315 |
| 10 | GYYWS | EIDHSGSTNYNPSLKS | ARGPWSFDP | RASQSIGSWLA | KASSLES | QQYDIYPT |
| | SEQ ID | SEQ ID NO: 3 | SEQ ID | SEQ ID | SEQ ID | SEQ ID |
| | NO: 298 | | NO: 299 | NO: 306 | NO: 301 | NO: 316 |
| 11 | GYYWS | EIDHSGSTNYNPSLKS | ARGPWSFDP | RASQSISSWLA | KASSLES | QQYDSYPT |
| | SEQ ID | SEQ ID NO: 3 | SEQ ID | SEQ ID | SEQ ID | SEQ ID |
| | NO: 298 | | NO: 299 | NO: 300 | NO: 301 | NO: 317 |
| 12 | GYYWS | EIDHSGSTNYNPSLKS | ARGPWSFDP | RASQSISSWLA | KASSLES | QQYGSFPT |
| | SEQ ID | SEQ ID NO: 3 | SEQ ID | SEQ ID | SEQ ID | SEQ ID |
| | NO: 298 | | NO: 299 | NO: 300 | NO: 301 | NO: 318 |
| 13 | GYYWS | EIDHSGSTNYNPSLKS | ARGPWSFDP | RASQSISSWLA | KASSLES | QQYQSFPT |
| | SEQ ID | SEQ ID NO: 3 | SEQ ID | SEQ ID | SEQ ID | SEQ ID |
| | NO: 298 | | NO: 299 | NO: 300 | NO: 301 | NO: 319 |
| 14 | GYYWS | EIDHSGSTNYNPSLKS | ARGPWSFDP | RASQSISSWLA | KASSLES | QQYSSFST |
| | SEQ ID | SEQ ID NO: 3 | SEQ ID | SEQ ID | SEQ ID | SEQ ID |
| | NO: 298 | | NO: 299 | NO: 300 | NO: 301 | NO: 320 |
| 15 | GYYWS | EIDHSGSTNYNPSLKS | ARGPWSFDP | RASQSISSWLA | KASSLES | QQYESYST |
| | SEQ ID | SEQ ID NO: 3 | SEQ ID | SEQ ID | SEQ ID | SEQ ID |
| | NO: 298 | | NO: 299 | NO: 300 | NO: 301 | NO: 321 |
| 16 | GYYWS | EIDHSGSTNYNPSLKS | ARGPWSFDP | RASQSISSWLA | KASSLES | QQYDSFIT |
| | SEQ ID | SEQ ID NO: 3 | SEQ ID | SEQ ID | SEQ ID | SEQ ID |
| | NO: 298 | | NO: 299 | NO: 300 | NO: 301 | NO: 322 |
| 17 | GYYWS | EIDHSGSTNYNPSLKS | ARGPWSFDP | RASQSISSWLA | KASSLES | QQYQSYPT |
| | SEQ ID | SEQ ID NO: 3 | SEQ ID | SEQ ID | SEQ ID | SEQ ID |
| | NO: 298 | | NO: 299 | NO: 300 | NO: 301 | NO: 323 |
| 18 | GYYWS | EIDHSGSTNYNPSLKS | ARGPWSFDP | RASQSIGSWLA | KASSLES | QQYHSFPT |
| | SEQ ID | SEQ ID NO: 3 | SEQ ID | SEQ ID | SEQ ID | SEQ ID |
| | NO: 298 | | NO: 299 | NO: 306 | NO: 301 | NO: 324 |
| 19 | GYYWS | EIDHSGSTNYNPSLKS | ARGPWSFDP | RASQSIGSWLA | KASSLES | QQYELYSYT |
| | SEQ ID | SEQ ID NO: 3 | SEQ ID | SEQ ID | SEQ ID | SEQ ID |
| | NO: 298 | | NO: 299 | NO: 306 | NO: 301 | NO: 325 |
| 20 | GYYWS | EIDHSGSTNYNPSLKS | ARGPWSFDP | RASQSISSWLA | KASSLES | QQYDTFIT |
| | SEQ ID | SEQ ID NO: 3 | SEQ ID | SEQ ID | SEQ ID | SEQ ID |
| | NO: 298 | | NO: 299 | NO: 300 | NO:301 | NO: 326 |
| 21 | SYAIS | GIIPIFGTANYAQKFQG | GDSSIRHAYYYYG | KSSQSVLYSSNNK | WASTRES | QQYYSTPIT |
| | SEQ ID | SEQ ID NO: 29 | MDV | NYLA | SEQ ID | SEQ ID |
| | NO: 28 | | SEQ ID | SEQ ID | NO: 34 | NO: 35 |
| | | | NO: 31 | NO: 33 | | |
| 22 | SSSYYWG | SIYYSGSTYYNPSLKS | GSDRFHPYFDY | RASQSVSRYLA | DASNRAT | QQFDTWPPT |
| | SEQ ID | SEQ ID NO: 39 | SEQ ID | SEQ ID | SEQ ID | SEQ ID |
| | NO: 38 | | NO: 41 | NO: 43 | NO: 44 | NO: 45 |
| 23 | GYYWS | EIDHSGSTNYNPSLKS | ARGPWSFDP | RASQSIGSWLA | KASSLES | EQYDSYPT |
| | SEQ ID | SEQ ID NO: 3 | SEQ ID | SEQ ID | SEQ ID | SEQ ID |
| | NO: 298 | | NO: 299 | NO: 306 | NO: 301 | NO: 327 |
| 24 | SYAIS | GIIPIFGTANYAQKFQG | RGRKASGSFYYYY | ESSQSLLNSGNQK | WASTRES | QNDYSYPYT |
| | SEQ ID | SEQ ID NO: 29 | GMDV | NYLT | SEQ ID | SEQ ID |
| | NO: 28 | | SEQ ID | SEQ ID | NO: 34 | NO: 51 |
| | | | NO: 328 | NO: 50 | | |
| 25 | SYYMH | IINPSGGSTSYAQKFQG | GAPNYGDTTHDYY | RASQSVSSNLA | GASTRAT | QQYDDWPFT |
| | SEQ ID | SEQ ID NO: 55 | YMDV | SEQ ID | SEQ ID | SEQ ID |
| | NO: 54 | | SEQ ID | NO: 59 | NO: 60 | NO: 61 |
| | | | NO: 57 | | | |
| 26 | GYYMH | WINPNSGGTNYAQKFQG | DTGEYYDTDDHGM | RASQSVSSNLA | GASTRAT | QQDDYWPPT |
| | SEQ ID | SEQ ID NO: 65 | DV | SEQ ID | SEQ ID | SEQ ID |
| | NO: 64 | | SEQ ID | NO: 59 | NO: 60 | NO: 69 |
| | | | NO: 67 | | | |
| 27 | SYAMS | AISGSGGSTYYADSVKG | DGGYYDSGAGDY | RASQGIDSWLA | AASSLQS | QQGVSYPRT |
| | SEQ ID | SEQ ID NO: 72 | SEQ ID | SEQ ID | SEQ ID | SEQ ID |
| | NO: 115 | | NO: 74 | NO: 76 | NO: 77 | NO: 78 |
| 28 | SYYMH | IINPSGGSTSYAQKFQG | EGAGFAYGMDYYY | RASQSVSSYLA | DASNRAT | QQSDNWPFT |
| | SEQ ID | SEQ ID NO: 55 | MDV | SEQ ID | SEQ ID | SEQ ID |
| | NO: 54 | | SEQ ID | NO: 93 | NO: 44 | NO: 94 |
| | | | NO: 91 | | | |
| 29 | SYSMN | SISSSSSYIYYADSVKG | GAPXGAAAGWFDP | RASQGISSWLA | AASSLQS | QQGVSFPRT |
| | SEQ ID | SEQ ID NO: 82 | X = M, I, Q, | SEQ ID | SEQ ID | SEQ ID |
| | NO: 81 | | L, F, V | NO: 86 | NO: 77 | NO: 87 |
| | | | SEQ ID | | | |
| | | | NO: 112 | | | |
| 30 | SYSMN | SISSSSSYIYYADSVKG | GAPMGAAAGWFDP | RASQGISSWLA | AASSLQS | QQGVSFPRT |
| | SEQ ID | SEQ ID NO: 82 | SEQ ID | SEQ ID | SEQ ID | SEQ ID |
| | NO: 81 | | NO: 84 | NO: 86 | NO: 77 | NO: 87 |
| 31 | SYSMN | SISSSSSYIYYADSVKG | GAPIGAAAGWFDP | RASQGISSWLA | AASSLQS | QQGVSFPRT |
| | SEQ ID | SEQ ID NO: 82 | SEQ ID | SEQ ID | SEQ ID | SEQ ID |
| | NO: 81 | | NO: 97 | NO: 86 | NO: 77 | NO: 87 |
| 32 | SYSMN | SISSSSSYIYYADSVKG | GAPQGAAAGWFDP | RASQGISSWLA | AASSLQS | QQGVSFPRT |
| | SEQ ID | SEQ ID NO: 82 | SEQ ID | SEQ ID | SEQ ID | SEQ ID |
| | NO: 81 | | NO: 100 | NO: 86 | NO: 77 | NO: 87 |
| 33 | SYSMN | SISSSSSYIYYADSVKG | GAPLGAAAGWFDP | RASQGISSWLA | AASSLQS | QQGVSFPRT |
| | SEQ ID | SEQ ID NO: 82 | SEQ ID | SEQ ID | SEQ ID | SEQ ID |
| | NO: 81 | | NO: 103 | NO: 86 | NO: 77 | NO: 87 |
| 34 | SYSMN | SISSSSSYIYYADSVKG | GAPFGAAAGWFDP | RASQGISSWLA | AASSLQS | QQGVSFPRT |
| | SEQ ID | SEQ ID NO: 82 | SEQ ID | SEQ ID | SEQ ID | SEQ ID |
| | NO: 81 | | NO: 106 | NO: 86 | NO: 77 | NO: 87 |
| 35 | SYSMN | SISSSSSYIYYADSVKG | GAPVGAAAGWFDP | RASQGISSWLA | AASSLQS | QQGVSFPRT |
| | SEQ ID | SEQ ID NO: 82 | SEQ ID | SEQ ID | SEQ ID | SEQ ID |
| | NO: 81 | | NO: 109 | NO: 86 | NO: 77 | NO: 87 |

TABLE 1B
CDRs for illustrative anti-NKG2D binding antigen binding
domains (Chothia)

| Ab | VH - | VH - | VH - | VL - | VL - | VL - |
|---|---|---|---|---|---|---|
| Name | CDR1 | CDR2 | CDR3 | CDR1 | CDR2 | CDR3 |
| 36 | GGSFSGY | HSG | RGPWSFD | SQSISSW | KAS | YNSYPI |
| | SEQ ID | SEQ ID | SEQ ID | SEQ ID | SEQ ID | SEQ ID |
| | NO: 329 | NO: 330 | NO: 331 | NO: 332 | NO: 333 | NO: 334 |
| 37 | GGSFSGY | HSG | RGPWSFD | SQSVSSSY | GAS | YGSSPI |
| | SEQ ID | SEQ ID | SEQ ID | SEQ ID | SEQ ID | SEQ ID |
| | NO: 329 | NO: 330 | NO: 331 | NO: 335 | NO: 336 | NO: 337 |
| 38 | GGSFSGY | HSG | RGPWSFD | SQSIGSW | KAS | YHSEY |
| | SEQ ID | SEQ ID | SEQ ID | SEQ ID | SEQ ID | SEQ ID |
| | NO: 329 | NO: 330 | NO: 331 | NO: 338 | NO: 333 | NO: 339 |
| 39 | GGSFSGY | HSG | RGPWSFD | SQSIGSW | KAS | SNSYY |
| | SEQ ID | SEQ ID | SEQ ID | SEQ ID | SEQ ID | SEQ ID |
| | NO: 329 | NO: 330 | NO: 331 | NO: 338 | NO: 333 | NO: 340 |
| 40 | GGSFSGY | HSG | RGPWSFD | SQSISSW | KAS | YNSYP |
| | SEQ ID | SEQ ID | SEQ ID | SEQ ID | SEQ ID | SEQ ID |
| | NO: 329 | NO: 330 | NO: 331 | NO: 332 | NO: 333 | NO: 341 |
| 41 | GGSFSGY | HSG | RGPWGFD | SQSISSY | WAS | SYDIPY |
| | SEQ ID | SEQ ID | SEQ ID | SEQ ID | SEQ ID | SEQ ID |
| | NO: 329 | NO: 330 | NO: 551 | NO: 342 | NO: 343 | NO: 344 |
| 42 | GGSFSGY | HSG | RGPWSFD | SQSISSW | KAS | YGSFPI |
| | SEQ ID | SEQ ID | SEQ ID | SEQ ID | SEQ ID | SEQ ID |
| | NO: 329 | NO: 330 | NO: 331 | NO: 332 | NO: 333 | NO: 345 |
| 43 | GGSFSGY | HSG | RGPWSFD | SQSISSW | KAS | SKEVPW |
| | SEQ ID | SEQ ID | SEQ ID | SEQ ID | SEQ ID | SEQ ID |
| | NO: 329 | NO: 330 | NO: 331 | NO: 332 | NO: 333 | NO: 346 |
| 44 | GGSFSGY | HSG | RGPWSFD | SQSISSW | KAS | YNSFP |
| | SEQ ID | SEQ ID | SEQ ID | SEQ ID | SEQ ID | SEQ ID |
| | NO: 329 | NO: 330 | NO: 331 | NO: 332 | NO: 333 | NO: 347 |
| 45 | GGSFSGY | HSG | RGPWSFD | SQSIGSW | KAS | YDIYP |
| | SEQ ID | SEQ ID | SEQ ID | SEQ ID | SEQ ID | SEQ ID |
| | NO: 329 | NO: 330 | NO: 331 | NO: 338 | NO: 333 | NO: 348 |
| 46 | GGSFSGY | HSG | RGPWSFD | SQSISSW | KAS | YDSYP |
| | SEQ ID | SEQ ID | SEQ ID | SEQ ID | SEQ ID | SEQ ID |
| | NO: 329 | NO: 330 | NO: 331 | NO: 332 | NO: 333 | NO: 349 |
| 47 | GGSFSGY | HSG | RGPWSFD | SQSISSW | KAS | YGSFP |
| | SEQ ID | SEQ ID | SEQ ID | SEQ ID | SEQ ID | SEQ ID |
| | NO: 329 | NO: 330 | NO: 331 | NO: 332 | NO: 333 | NO: 350 |
| 47 | GGSFSGY | HSG | RGPWSFD | SQSISSW | KAS | YQSFP |
| | SEQ ID | SEQ ID | SEQ ID | SEQ ID | SEQ ID | SEQ ID |
| | NO: 329 | NO: 330 | NO: 331 | NO: 332 | NO: 333 | NO: 351 |
| 48 | GGSFSGY | HSG | RGPWSFD | SQSISSW | KAS | YSSFS |
| | SEQ ID | SEQ ID | SEQ ID | SEQ ID | SEQ ID | SEQ ID |
| | NO: 329 | NO: 330 | NO: 331 | NO: 332 | NO: 333 | NO: 352 |
| 49 | GGSFSGY | HSG | RGPWSFD | SQSISSW | KAS | YESYS |
| | SEQ ID | SEQ ID | SEQ ID | SEQ ID | SEQ ID | SEQ ID |
| | NO: 329 | NO: 330 | NO: 331 | NO: 332 | NO: 333 | NO: 353 |
| 50 | GGSFSGY | HSG | RGPWSFD | SQSISSW | KAS | YDSEI |
| | SEQ ID | SEQ ID | SEQ ID | SEQ ID | SEQ ID | SEQ ID |
| | NO: 329 | NO: 330 | NO: 331 | NO: 332 | NO: 333 | NO: 354 |
| 51 | GGSFSGY | HSG | RGPWSFD | SQSISSW | KAS | YQSYP |
| | SEQ ID | SEQ ID | SEQ ID | SEQ ID | SEQ ID | SEQ ID |
| | NO: 329 | NO: 330 | NO: 331 | NO: 332 | NO: 333 | NO: 355 |
| 52 | GGSFSGY | HSG | RGPWSFD | SQSIGSW | KAS | YHSFP |
| | SEQ ID | SEQ ID | SEQ ID | SEQ ID | SEQ ID | SEQ ID |
| | NO: 329 | NO: 330 | NO: 331 | NO: 338 | NO: 333 | NO: 356 |
| 53 | GGSFSGY | HSG | RGPWSFD | SQSIGSW | KAS | YELYSY |
| | SEQ ID | SEQ ID | SEQ ID | SEQ ID | SEQ ID | SEQ ID |
| | NO: 329 | NO: 330 | NO: 331 | NO: 338 | NO: 333 | NO: 357 |
| 54 | GGSFSGY | HSG | RGPWSFD | SQSISSW | KAS | YDTFI |
| | SEQ ID | SEQ ID | SEQ ID | SEQ ID | SEQ ID | SEQ ID |
| | NO: 329 | NO: 330 | NO: 331 | NO: 332 | NO: 333 | NO: 358 |
| 55 | GGTFSSY | PIFG | DSSIRHAYYYYGMD | SQSVLYSSNNKNY | WAS | YYSTPI |
| | SEQ ID | SEQ ID | SEQ ID | SEQ ID | SEQ ID | SEQ ID |
| | NO: 359 | NO: 360 | NO: 361 | NO: 362 | NO: 343 | NO: 363 |
| 56 | GGSISSSSY | YSG | SDRFHPYFD | SQSVSRY | DAS | FDTWPP |
| | SEQ ID | SEQ ID | SEQ ID | SEQ ID | SEQ ID | SEQ ID |
| | NO: 364 | NO: 365 | NO: 366 | NO: 367 | NO: 368 | NO: 369 |
| 57 | GGSFSGY | HSG | RGPWSFD | SQSISSW | KAS | YDSYP |
| | SEQ ID | SEQ ID | SEQ ID | SEQ ID | SEQ ID | SEQ ID |
| | NO: 329 | NO: 330 | NO: 331 | NO: 332 | NO: 333 | NO: 349 |
| 58 | GGTFSSY | PIFG | GRKASGSFYYYYGMD | SQSLLNSGNQKNY | WAS | DYSYPY |
| | SEQ ID | SEQ ID | SEQ ID | SEQ ID | SEQ ID | SEQ ID |
| | NO: 359 | NO: 360 | NO: 370 | NO: 371 | NO: 343 | NO: 372 |
| 59 | GYTFTSY | PSGG | APNYGDTTHDYYYMD | SQSVSSN | GAS | YDDWPF |
| | SEQ ID | SEQ ID | SEQ ID | SEQ ID | SEQ ID | SEQ ID |
| | NO: 186 | NO: 373 | NO: 374 | NO: 375 | NO: 336 | NO: 376 |
| 60 | GYTFTGY | PNSG | TGEYYDTDDHGMD | SQSVSSN | GAS | DDYWPP |
| | SEQ ID | SEQ ID | SEQ ID | SEQ ID | SEQ ID | SEQ ID |
| | NO: 377 | NO: 378 | NO: 379 | NO: 375 | NO: 336 | NO: 380 |
| 61 | GFTFSSY | GSGG | GGYYDSGAGD | SQGIDSW | AAS | GVSYPR |
| | SEQ ID | SEQ ID | SEQ ID | SEQ ID | SEQ ID | SEQ ID |
| | NO: 381 | NO: 382 | NO: 383 | NO: 384 | NO: 385 | NO: 386 |
| 62 | GYTFTSY | PSGG | GAGFAYGMDYYYMD | SQSVSSY | DAS | SDNWPF |
| | SEQ ID | SEQ ID | SEQ ID | SEQ ID | SEQ ID | SEQ ID |
| | NO: 186 | NO: 373 | NO: 387 | NO: 388 | NO: 368 | NO: 389 |
| 63 | GFTFSSY | SSSS | APXGAAAGWFD | SQGISSW | AAS | GVSFPR |
| | SEQ ID | SEQ ID | X = M, I, Q, | SEQ ID | SEQ ID | SEQ ID |
| | NO: 381 | NO: 390 | L, F, V | NO: 392 | NO: 385 | NO: 393 |
| | | | SEQ ID | | | |
| | | | NO: 391 | | | |
| 64 | GFTFSSY | SSSS | APMGAAAGWFD | SQGISSW | AAS | GVSFPR |
| | SEQ ID | SEQ ID | SEQ ID | SEQ ID | SEQ ID | SEQ ID |
| | NO: 381 | NO: 390 | NO: 394 | NO: 392 | NO: 385 | NO: 393 |
| 65 | GFTFSSY | SSSS | APIGAAAGWFD | SQGISSW | AAS | GVSFPR |
| | SEQ ID | SEQ ID | SEQ ID | SEQ ID | SEQ ID | SEQ ID |
| | NO: 381 | NO: 390 | NO: 395 | NO: 392 | NO: 385 | NO: 393 |
| 66 | GFTFSSY | SSSS | APQGAAAGWFD | SQGISSW | AAS | GVSFPR |
| | SEQ ID | SEQ ID | SEQ ID | SEQ ID | SEQ ID | SEQ ID |
| | NO: 381 | NO: 390 | NO: 396 | NO: 392 | NO: 385 | NO: 393 |
| 67 | GFTFSSY | SSSS | APLGAAAGWFD | SQGISSW | AAS | GVSFPR |
| | SEQ ID | SEQ ID | SEQ ID | SEQ ID | SEQ ID | SEQ ID |
| | NO: 381 | NO: 390 | NO: 397 | NO: 392 | NO: 385 | NO: 393 |
| 68 | GFTFSSY | SSSS | APFGAAAGWFD | SQGISSW | AAS | GVSFPR |
| | SEQ ID | SEQ ID | SEQ ID | SEQ ID | SEQ ID | SEQ ID |
| | NO: 381 | NO: 390 | NO: 398 | NO: 392 | NO: 385 | NO: 393 |
| 69 | GFTFSSY | SSSS | APVGAAAGWFD | SQGISSW | AAS | GVSFPR |
| | SEQ ID | SEQ ID | SEQ ID | SEQ ID | SEQ ID | SEQ ID |
| | NO: 381 | NO: 390 | NO: 399 | NO: 392 | NO: 385 | NO: 393 |

TABLE 1C
CDRs for illustrative anti-NKG2D binding antigen binding
domains (IMGT)

| Ab | VH - | VH - | VH - | VL - | VL - | VL - |
|---|---|---|---|---|---|---|
| Name | CDR1 | CDR2 | CDR3 | CDR1 | CDR2 | CDR3 |
| 70 | GGSFSGYY | IDHSGST | ARARGPWSFDP | QSISSW | KAS | QQYNSYPIT |
| | SEQ ID | SEQ ID | SEQ ID | SEQ ID | SEQ ID | SEQ ID |
| | NO: 400 | NO: 401 | NO: 4 | NO: 402 | NO: 333 | NO: 302 |
| 71 | GGSFSGYY | IDHSGST | ARARGPWSFDP | QSVSSSY | GAS | QQYGSSPIT |
| | SEQ ID | SEQ ID | SEQ ID | SEQ ID | SEQ ID | SEQ ID |
| | NO: 400 | NO: 401 | NO: 4 | NO: 552 | NO: 336 | NO: 305 |
| 72 | GGSFSGYY | IDHSGST | ARARGPWSFDP | QSIGSW | KAS | QQYHSFYT |
| | SEQ ID | SEQ ID | SEQ ID | SEQ ID | SEQ ID | SEQ ID |
| | NO: 400 | NO: 401 | NO: 4 | NO: 403 | NO: 333 | NO: 307 |
| 73 | GGSFSGYY | IDHSGST | ARARGPWSFDP | QSIGSW | KAS | QQSNSYYT |
| | SEQ ID | SEQ ID | SEQ ID | SEQ ID | SEQ ID | SEQ ID |
| | NO: 400 | NO: 401 | NO: 4 | NO: 403 | NO: 333 | NO: 308 |
| 74 | GGSFSGYY | IDHSGST | ARARGPWSFDP | QSISSW | KAS | QQYNSYPT |
| | SEQ ID | SEQ ID | SEQ ID | SEQ ID | SEQ ID | SEQ ID |
| | NO: 400 | NO: 401 | NO: 4 | NO: 402 | NO: 333 | NO: 309 |
| 75 | GGSFSGYY | IDHSGST | ARARGPWGFDP | QSISSY | WAS | QQSYDIPYT |
| | SEQ ID | SEQ ID | SEQ ID | SEQ ID | SEQ ID | SEQ ID |
| | NO: 400 | NO: 401 | NO: 404 | NO: 405 | NO: 343 | NO: 312 |
| 76 | GGSFSGYY | IDHSGST | ARARGPWSFDP | QSISSW | KAS | QQYGSFPIT |
| | SEQ ID | SEQ ID | SEQ ID | SEQ ID | SEQ ID | SEQ ID |
| | NO: 400 | NO: 401 | NO: 4 | NO: 402 | NO: 333 | NO: 313 |
| 77 | GGSFSGYY | IDHSGST | ARARGPWSFDP | QSISSW | KAS | QQSKEVPWT |
| | SEQ ID | SEQ ID | SEQ ID | SEQ ID | SEQ ID | SEQ ID |
| | NO: 400 | NO: 401 | NO: 4 | NO: 402 | NO: 333 | NO: 314 |
| 78 | GGSFSGYY | IDHSGST | ARARGPWSFDP | QSISSW | KAS | QQYNSFPT |
| | SEQ ID | SEQ ID | SEQ ID | SEQ ID | SEQ ID | SEQ ID |
| | NO: 400 | NO: 401 | NO: 4 | NO: 402 | NO: 333 | NO: 315 |
| 79 | GGSFSGYY | IDHSGST | ARARGPWSFDP | QSIGSW | KAS | QQYDIYPT |
| | SEQ ID | SEQ ID | SEQ ID | SEQ ID | SEQ ID | SEQ ID |
| | NO: 400 | NO: 401 | NO: 4 | NO: 403 | NO: 333 | NO: 316 |
| 80 | GGSFSGYY | IDHSGST | ARARGPWSFDP | QSISSW | KAS | QQYDSYPT |
| | SEQ ID | SEQ ID | SEQ ID | SEQ ID | SEQ ID | SEQ ID |
| | NO: 400 | NO: 401 | NO: 4 | NO: 402 | NO: 333 | NO: 317 |
| 81 | GGSFSGYY | IDHSGST | ARARGPWSFDP | QSISSW | KAS | QQYGSFPT |
| | SEQ ID | SEQ ID | SEQ ID | SEQ ID | SEQ ID | SEQ ID |
| | NO: 400 | NO: 401 | NO: 4 | NO: 402 | NO: 333 | NO: 318 |
| 82 | GGSFSGYY | IDHSGST | ARARGPWSFDP | QSISSW | KAS | QQYQSFPT |
| | SEQ ID | SEQ ID | SEQ ID | SEQ ID | SEQ ID | SEQ ID |
| | NO: 400 | NO: 401 | NO: 4 | NO: 402 | NO: 333 | NO: 319 |
| 83 | GGSFSGYY | IDHSGST | ARARGPWSFDP | QSISSW | KAS | QQYSSFST |
| | SEQ ID | SEQ ID | SEQ ID | SEQ ID | SEQ ID | SEQ ID |
| | NO: 400 | NO: 401 | NO: 4 | NO: 402 | NO: 333 | NO: 320 |
| 84 | GGSFSGYY | IDHSGST | ARARGPWSFDP | QSISSW | KAS | QQYESYST |
| | SEQ ID | SEQ ID | SEQ ID | SEQ ID | SEQ ID | SEQ ID |
| | NO: 400 | NO: 401 | NO: 4 | NO: 402 | NO: 333 | NO: 321 |
| 85 | GGSFSGYY | IDHSGST | ARARGPWSFDP | QSISSW | KAS | QQYDSFIT |
| | SEQ ID | SEQ ID | SEQ ID | SEQ ID | SEQ ID | SEQ ID |
| | NO: 400 | NO: 401 | NO: 4 | NO: 402 | NO: 333 | NO: 322 |
| 86 | GGSFSGYY | IDHSGST | ARARGPWSFDP | QSISSW | KAS | QQYQSYPT |
| | SEQ ID | SEQ ID | SEQ ID | SEQ ID | SEQ ID | SEQ ID |
| | NO: 400 | NO: 401 | NO: 4 | NO: 402 | NO: 333 | NO: 323 |
| 87 | GGSFSGYY | IDHSGST | ARARGPWSFDP | QSIGSW | KAS | QQYHSFPT |
| | SEQ ID | SEQ ID | SEQ ID | SEQ ID | SEQ ID | SEQ ID |
| | NO: 400 | NO: 401 | NO: 4 | NO: 403 | NO: 333 | NO: 324 |
| 88 | GGSFSGYY | IDHSGST | ARARGPWSFDP | QSIGSW | KAS | QQYELYSYT |
| | SEQ ID | SEQ ID | SEQ ID | SEQ ID | SEQ ID | SEQ ID |
| | NO: 400 | NO: 401 | NO: 4 | NO: 403 | NO: 333 | NO: 325 |
| 89 | GGSFSGYY | IDHSGST | ARARGPWSFDP | QSISSW | KAS | QQYDTFIT |
| | SEQ ID | SEQ ID | SEQ ID | SEQ ID | SEQ ID | SEQ ID |
| | NO: 400 | NO: 401 | NO: 4 | NO: 402 | NO: 333 | NO: 326 |
| 90 | GGTFSSYA | IIPIFGTA | ARGDSSIRHAYYYYGMDV | QSVLYSSNNKNY | WAS | QQYYSTPIT |
| | SEQ ID | SEQ ID | SEQ ID | SEQ ID | SEQ ID | SEQ ID |
| | NO: 406 | NO: 407 | NO: 30 | NO: 408 | NO: 343 | NO: 35 |
| 91 | GGSISSSSYY | IYYSGST | ARGSDRFHPYFDY | QSVSRY | DAS | QQFDTWPPT |
| | SEQ ID | SEQ ID | SEQ ID | SEQ ID | SEQ ID | SEQ ID |
| | NO: 409 | NO: 410 | NO: 40 | NO: 411 | NO: 368 | NO: 45 |
| 92 | GGSFSGYY | IDHSGST | ARARGPWSFDP | QSISSW | KAS | EQYDSYPT |
| | SEQ ID | SEQ ID | SEQ ID | SEQ ID | SEQ ID | SEQ ID |
| | NO: 400 | NO: 401 | NO: 4 | NO: 402 | NO: 333 | NO: 327 |
| 93 | GGTFSSYA | IIPIFGTA | ARRGRKASGSFYYYYGMDV | QSLLNSGNQKNY | WAS | QNDYSYPYT |
| | SEQ ID | SEQ ID | SEQ ID | SEQ ID | SEQ ID | SEQ ID |
| | NO: 406 | NO: 407 | NO: 48 | NO: 412 | NO: 343 | NO: 51 |
| 94 | GYTFTSYY | INPSGGST | ARGAPNYGDTTHDYYYMDV | QSVSSN | GAS | QQYDDWPFT |
| | SEQ ID | SEQ ID | SEQ ID | SEQ ID | SEQ ID | SEQ ID |
| | NO: 413 | NO: 414 | NO: 56 | NO: 415 | NO: 336 | NO: 61 |
| 95 | GYTFTGYY | INPNSGGT | ARDTGEYYDTDDHGMDV | QSVSSN | GAS | QQDDYWPPT |
| | SEQ ID | SEQ ID | SEQ ID | SEQ ID | SEQ ID | SEQ ID |
| | NO: 416 | NO: 417 | NO: 66 | NO: 415 | NO: 336 | NO: 69 |
| 96 | GFTFSSYA | ISGSGGST | AKDGGYYDSGAGDY | QGIDSW | AAS | QQGVSYPRT |
| | SEQ ID | SEQ ID | SEQ ID | SEQ ID | SEQ ID | SEQ ID |
| | NO: 418 | NO: 419 | NO: 73 | NO: 420 | NO: 385 | NO: 78 |
| 97 | GYTFTSYY | INPSGGST | AREGAGFAYGMDYYYMDV | QSVSSY | DAS | QQSDNWPFT |
| | SEQ ID | SEQ ID | SEQ ID | SEQ ID | SEQ ID | SEQ ID |
| | NO: 413 | NO: 414 | NO: 90 | NO: 421 | NO: 368 | NO:94 |
| 98 | GFTFSSYS | ISSSSSYI | ARGAPXGAAAGWFDP | QGISSW | AAS | QQGVSFPRT |
| | SEQ ID | SEQ ID | X = M, I, Q, | SEQ ID | SEQ ID | SEQ ID |
| | NO: 422 | NO: 423 | L, F, V | NO: 424 | NO: 385 | NO: 87 |
| | | | SEQ ID | | | |
| | | | NO: 111 | | | |
| 99 | GFTFSSYS | ISSSSSYI | ARGAPMGAAAGWFDP | QGISSW | AAS | QQGVSFPRT |
| | SEQ ID | SEQ ID | SEQ ID | SEQ ID | SEQ ID | SEQ ID |
| | NO: 422 | NO: 423 | NO: 83 | NO: 424 | NO: 385 | NO: 87 |
| 100 | GFTFSSYS | ISSSSSYI | ARGAPIGAAAGWFDP | QGISSW | AAS | QQGVSFPRT |
| | SEQ ID | SEQ ID | SEQ ID | SEQ ID | SEQ ID | SEQ ID |
| | NO: 422 | NO: 423 | NO: 96 | NO: 424 | NO: 385 | NO: 87 |
| 101 | GFTFSSYS | ISSSSSYI | ARGAPQGAAAGWFDP | QGISSW | AAS | QQGVSFPRT |
| | SEQ ID | SEQ ID | SEQ ID | SEQ ID | SEQ ID | SEQ ID |
| | NO: 422 | NO: 423 | NO: 99 | NO: 424 | NO: 385 | NO: 87 |
| 102 | GFTFSSYS | ISSSSSYI | ARGAPLGAAAGWFDP | QGISSW | AAS | QQGVSFPRT |
| | SEQ ID | SEQ ID | SEQ ID | SEQ ID | SEQ ID | SEQ ID |
| | NO: 422 | NO: 423 | NO: 102 | NO: 424 | NO: 385 | NO: 87 |
| 103 | GFTFSSYS | ISSSSSYI | ARGAPFGAAAGWFDP | QGISSW | AAS | QQGVSFPRT |
| | SEQ ID | SEQ ID | SEQ ID | SEQ ID | SEQ ID | SEQ ID |
| | NO: 422 | NO: 423 | NO: 105 | NO: 424 | NO: 385 | NO: 87 |
| 104 | GFTFSSYS | ISSSSSYI | ARGAPVGAAAGWFDP | QGISSW | AAS | QQGVSFPRT |
| | SEQ ID | SEQ ID | SEQ ID | SEQ ID | SEQ ID | SEQ ID |
| | NO: 422 | NO: 423 | NO: 108 | NO: 424 | NO: 385 | NO: 87 |

TABLE 1D
CDRs for illustrative anti-NKG2D binding
antigen binding domains (Honegger)

| Ab | VH - | VH - | VH - | VL - | VL - | VL - |
|---|---|---|---|---|---|---|
| Name | CDR1 | CDR2 | CDR3 | CDR1 | CDR2 | CDR3 |
| 105 | VYGGSFSGYY | IDHSGSTNYNPSLKSR | ARGPWSFD | ASQSISSW | KASSLESGVPSR | YNSYPI |
| | SEQ ID | SEQ ID NO: 426 | SEQ ID | SEQ ID | SEQ ID | SEQ ID |
| | NO: 425 | | NO: 427 | NO: 428 | NO: 429 | NO: 334 |
| 106 | VYGGSFSGYY | IDHSGSTNYNPSLKSR | ARGPWSFD | ASQSVSSSY | GASSRATGIPDR | YGSSPI |
| | SEQ ID | SEQ ID NO: 426 | SEQ ID | SEQ ID | SEQ ID | SEQ ID |
| | NO: 425 | | NO: 427 | NO: 430 | NO: 431 | NO: 337 |
| 107 | VYGGSFSGYY | IDHSGSTNYNPSLKSR | ARGPWSFD | ASQSIGSW | KASSLESGVPSR | YHSEY |
| | SEQ ID | SEQ ID NO: 426 | SEQ ID | SEQ ID | SEQ ID | SEQ ID |
| | NO: 425 | | NO: 427 | NO: 432 | NO: 429 | NO: 339 |
| 108 | VYGGSFSGYY | IDHSGSTNYNPSLKSR | ARGPWSFD | ASQSIGSW | KASSLESGVPSR | SNSYY |
| | SEQ ID | SEQ ID NO: 426 | SEQ ID | SEQ ID | SEQ ID | SEQ ID |
| | NO: 425 | | NO: 427 | NO: 432 | NO: 429 | NO: 340 |
| 109 | VYGGSFSGYY | IDHSGSTNYNPSLKSR | ARGPWSFD | ASQSISSW | KASSLESGVPSR | YNSYP |
| | SEQ ID | SEQ ID NO: 426 | SEQ ID | SEQ ID | SEQ ID | SEQ ID |
| | NO: 425 | | NO: 427 | NO: 428 | NO: 429 | NO: 341 |
| 110 | VYGGSFSGYY | IDHSGSTNYNPSLKSR | ARGPWGFD | TSQSISSY | WASTRESGVPDR | SYDIPY |
| | SEQ ID | SEQ ID NO: 426 | SEQ ID | SEQ ID | SEQ ID | SEQ ID |
| | NO: 425 | | NO: 433 | NO: 434 | NO: 435 | NO: 344 |
| 111 | VYGGSFSGYY | IDHSGSTNYNPSLKSR | ARGPWSFD | ASQSISSW | KASSLESGVPSR | YGSFPI |
| | SEQ ID | SEQ ID NO: 426 | SEQ ID | SEQ ID | SEQ ID | SEQ ID |
| | NO: 425 | | NO: 427 | NO: 428 | NO: 429 | NO: 345 |
| 112 | VYGGSFSGYY | IDHSGSTNYNPSLKSR | ARGPWSFD | ASQSISSW | KASSLESGVPSR | SKEVPW |
| | SEQ ID | SEQ ID NO: 426 | SEQ ID | SEQ ID | SEQ ID | SEQ ID |
| | NO: 425 | | NO: 427 | NO: 428 | NO: 429 | NO: 346 |
| 113 | VYGGSFSGYY | IDHSGSTNYNPSLKSR | ARGPWSFD | ASQSISSW | KASSLESGVPSR | YNSFP |
| | SEQ ID | SEQ ID NO: 426 | SEQ ID | SEQ ID | SEQ ID | SEQ ID |
| | NO: 425 | | NO: 427 | NO: 428 | NO: 429 | NO: 347 |
| 114 | VYGGSFSGYY | IDHSGSTNYNPSLKSR | ARGPWSFD | ASQSIGSW | KASSLESGVPSR | YDIYP |
| | SEQ ID | SEQ ID NO: 426 | SEQ ID | SEQ ID | SEQ ID | SEQ ID |
| | NO: 425 | | NO: 427 | NO: 432 | NO: 429 | NO: 348 |
| 115 | VYGGSFSGYY | IDHSGSTNYNPSLKSR | ARGPWSFD | ASQSISSW | KASSLESGVPSR | YDSYP |
| | SEQ ID | SEQ ID NO: 426 | SEQ ID | SEQ ID | SEQ ID | SEQ ID |
| | NO: 425 | | NO: 427 | NO: 428 | NO: 429 | NO: 349 |
| 116 | VYGGSFSGYY | IDHSGSTNYNPSLKSR | ARGPWSFD | ASQSISSW | KASSLESGVPSR | YGSFP |
| | SEQ ID | SEQ ID NO: 426 | SEQ ID | SEQ ID | SEQ ID | SEQ ID |
| | NO: 425 | | NO: 427 | NO: 428 | NO: 429 | NO: 350 |
| 117 | VYGGSFSGYY | IDHSGSTNYNPSLKSR | ARGPWSFD | ASQSISSW | KASSLESGVPSR | YQSFP |
| | SEQ ID | SEQ ID NO: 426 | SEQ ID | SEQ ID | SEQ ID | SEQ ID |
| | NO: 425 | | NO: 427 | NO: 428 | NO: 429 | NO: 351 |
| 118 | VYGGSFSGYY | IDHSGSTNYNPSLKSR | ARGPWSFD | ASQSISSW | KASSLESGVPSR | YSSES |
| | SEQ ID | SEQ ID NO: 426 | SEQ ID | SEQ ID | SEQ ID | SEQ ID |
| | NO: 425 | | NO: 427 | NO: 428 | NO: 429 | NO: 352 |
| 119 | VYGGSFSGYY | IDHSGSTNYNPSLKSR | ARGPWSFD | ASQSISSW | KASSLESGVPSR | YESYS |
| | SEQ ID | SEQ ID NO: 426 | SEQ ID | SEQ ID | SEQ ID | SEQ ID |
| | NO: 425 | | NO: 427 | NO: 428 | NO: 429 | NO: 353 |
| 120 | VYGGSFSGYY | IDHSGSTNYNPSLKSR | ARGPWSFD | ASQSISSW | KASSLESGVPSR | YDSEI |
| | SEQ ID | SEQ ID NO: 426 | SEQ ID | SEQ ID | SEQ ID | SEQ ID |
| | NO: 425 | | NO: 427 | NO: 428 | NO: 429 | NO: 354 |
| 121 | VYGGSFSGYY | IDHSGSTNYNPSLKSR | ARGPWSFD | ASQSISSW | KASSLESGVPSR | YQSYP |
| | SEQ ID | SEQ ID NO: 426 | SEQ ID | SEQ ID | SEQ ID | SEQ ID |
| | NO: 425 | | NO: 427 | NO: 428 | NO: 429 | NO: 355 |
| 122 | VYGGSFSGYY | IDHSGSTNYNPSLKSR | ARGPWSFD | ASQSIGSW | KASSLESGVPSR | YHSFP |
| | SEQ ID | SEQ ID NO: 426 | SEQ ID | SEQ ID | SEQ ID | SEQ ID |
| | NO: 425 | | NO: 427 | NO: 432 | NO: 429 | NO: 356 |
| 123 | VYGGSFSGYY | IDHSGSTNYNPSLKSR | ARGPWSFD | ASQSIGSW | KASSLESGVPSR | YELYSY |
| | SEQ ID | SEQ ID NO: 426 | SEQ ID | SEQ ID | SEQ ID | SEQ ID |
| | NO: 425 | | NO: 427 | NO: 432 | NO: 429 | NO: 357 |
| 124 | VYGGSFSGYY | IDHSGSTNYNPSLKSR | ARGPWSFD | ASQSISSW | KASSLESGVPSR | YDTFI |
| | SEQ ID | SEQ ID NO: 426 | SEQ ID | SEQ ID | SEQ ID | SEQ ID |
| | NO: 425 | | NO: 427 | NO: 428 | NO: 429 | NO: 358 |
| 125 | ASGGTFSSYA | IIPIFGTANYAQKFQG | GDSSIRHA | SSQSVLYS | WASTRESGVPDR | YYSTPI |
| | SEQ ID | R | YYYYGMD | SNNKNY | SEQ ID | SEQ ID |
| | NO: 436 | SEQ ID NO: 437 | SEQ ID | SEQ ID | NO: 435 | NO: 363 |
| | | | NO: 438 | NO: 439 | | |
| 126 | VSGGSISSSSYY | IYYSGSTYYNPSLKSR | GSDRFHPYFD | ASQSVSRY | DASNRATGIPAR | FDTWPP |
| | SEQ ID | SEQ ID NO: 441 | SEQ ID | SEQ ID | SEQ ID | SEQ ID |
| | NO: 440 | | NO: 442 | NO: 443 | NO: 444 | NO: 369 |
| 127 | VYGGSFSGYY | IDHSGSTNYNPSLKSR | ARGPWSFD | ASQSISSW | KASSLESGVPSR | YDSYP |
| | SEQ ID | SEQ ID NO: 426 | SEQ ID | SEQ ID | SEQ ID | SEQ ID |
| | NO: 425 | | NO: 427 | NO: 428 | NO: 429 | NO: 349 |
| 128 | ASGGTFSSYA | IIPIFGTANYAQKFQG | RGRKASGSF | SSQSLLNS | WASTRESGVPDR | DYSYPY |
| | SEQ ID | R | YYYYGMD | GNQKNY | SEQ ID | SEQ ID |
| | NO: 436 | SEQ ID NO: 437 | SEQ ID | SEQ ID | NO: 435 | NO: 372 |
| | | | NO: 445 | NO: 446 | | |
| 129 | ASGYTFTSYY | INPSGGSTSYAQKFQG | GAPNYGDT | ASQSVSSN | GASTRATGIPAR | YDDWPF |
| | SEQ ID | R | THDYYYMD | SEQ ID | SEQ ID | SEQ ID |
| | NO: 447 | SEQ ID NO: 448 | SEQ ID | NO: 450 | NO: 451 | NO: 376 |
| | | | NO: 449 | | | |
| 130 | ASGYTFTGYY | INPNSGGTNYAQKFQG | DTGEYYDTD | ASQSVSSN | GASTRATGIPAR | DDYWPP |
| | SEQ ID | R | DHGMD | SEQ ID | SEQ ID | SEQ ID |
| | NO: 452 | SEQ ID NO: 453 | SEQ ID | NO: 450 | NO: 451 | NO: 380 |
| | | | NO: 454 | | | |
| 131 | ASGFTFSSYA | ISGSGGSTYYADSVKG | DGGYYDSGAGD | ASQGIDSW | AASSLQSGVPSR | GVSYPR |
| | SEQ ID | R | SEQ ID | SEQ ID | SEQ ID | SEQ ID |
| | NO: 455 | SEQ ID NO: 456 | NO: 457 | NO: 458 | NO: 459 | NO: 386 |
| 132 | ASGYTFTSYY | INPSGGSTSYAQKFQG | EGAGFAYGMDY | ASQSVSSY | DASNRATGIPAR | SDNWPF |
| | SEQ ID | R | YYMD | SEQ ID | SEQ ID | SEQ ID |
| | NO: 447 | SEQ ID NO: 448 | SEQ ID | NO: 461 | NO: 444 | NO: 389 |
| | | | NO: 460 | | | |
| 133 | ASGFTFSSYS | ISSSSSYIYYADSVKG | GAPXGAAAGWFD | ASQGISSW | AASSLQSGVPSR | GVSFPR |
| | SEQ ID | R | X = M, I, Q, | SEQ ID | SEQ ID | SEQ ID |
| | NO: 462 | SEQ ID NO: 463 | L, F, V | NO: 465 | NO: 459 | NO: 393 |
| | | | SEQ ID | | | |
| | | | NO: 464 | | | |
| 134 | ASGFTFSSYS | ISSSSSYIYYADSVKG | GAPMGAAAGWFD | ASQGISSW | AASSLQSGVPSR | GVSFPR |
| | SEQ ID | R | SEQ ID | SEQ ID | SEQ ID | SEQ ID |
| | NO: 462 | SEQ ID NO: 463 | NO: 466 | NO: 465 | NO: 459 | NO: 393 |
| 135 | ASGFTFSSYS | ISSSSSYIYYADSVKG | GAPIGAAAGWFD | ASQGISSW | AASSLQSGVPSR | GVSFPR |
| | SEQ ID | R | SEQ ID | SEQ ID | SEQ ID | SEQ ID |
| | NO: 462 | SEQ ID NO: 463 | NO: 467 | NO: 465 | NO: 459 | NO: 393 |
| 136 | ASGFTFSSYS | ISSSSSYIYYADSVKG | GAPQGAAAGWFD | ASQGISSW | AASSLQSGVPSR | GVSFPR |
| | SEQ ID | R | SEQ ID | SEQ ID | SEQ ID | SEQ ID |
| | NO: 462 | SEQ ID NO: 463 | NO: 468 | NO: 465 | NO: 459 | NO: 393 |
| 137 | ASGFTFSSYS | ISSSSSYIYYADSVKG | GAPLGAAAGWFD | ASQGISSW | AASSLQSGVPSR | GVSFPR |
| | SEQ ID | R | SEQ ID | SEQ ID | SEQ ID | SEQ ID |
| | NO: 462 | SEQ ID NO: 463 | NO: 469 | NO: 465 | NO: 459 | NO: 393 |
| 138 | ASGFTFSSYS | ISSSSSYIYYADSVKG | GAPFGAAAGWFD | ASQGISSW | AASSLQSGVPSR | GVSFPR |
| | SEQ ID | R | SEQ ID | SEQ ID | SEQ ID | SEQ ID |
| | NO: 462 | SEQ ID NO: 463 | NO: 470 | NO: 465 | NO: 459 | NO: 393 |
| 139 | ASGFTFSSYS | ISSSSSYIYYADSVKG | GAPVGAAAGWFD | ASQGISSW | AASSLQSGVPSR | GVSFPR |
| | SEQ ID | R | SEQ ID | SEQ ID | SEQ ID | SEQ ID |
| | NO: 462 | SEQ ID NO: 463 | NO: 471 | NO: 465 | NO: 459 | NO: 393 |

In various embodiments, the first antigen-binding site or first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively:

SEQ ID NOs: 298, 3, 299, 300, 301 and 302;

SEQ ID NOs: 298, 3, 299, 303, 304 and 305;

SEQ ID NOs: 298, 3, 299, 306, 301 and 307;

SEQ ID NOs: 298, 3, 299, 306, 301 and 308;

SEQ ID NOs: 298, 3, 299, 300, 301 and 309;

SEQ ID NOs: 298, 3, 310, 311, 34 and 312;

SEQ ID NOs: 298, 3, 299, 300, 301 and 313;

SEQ ID NOs: 298, 3, 299, 300, 301 and 314;

SEQ ID NOs: 298, 3, 299, 300, 301 and 315;

SEQ ID NOs: 298, 3, 299, 306, 301 and 316;

SEQ ID NOs: 298, 3, 299, 300, 301 and 317;

SEQ ID NOs: 298, 3, 299, 300, 301 and 318;

SEQ ID NOs: 298, 3, 299, 300, 301 and 319;

SEQ ID NOs: 298, 3, 299, 300, 301 and 320;

SEQ ID NOs: 298, 3, 299, 300, 301 and 321;

SEQ ID NOs: 298, 3, 299, 300, 301 and 322;

SEQ ID NOs: 298, 3, 299, 300, 301 and 323;

SEQ ID NOs: 298, 3, 299, 306, 301 and 324;

SEQ ID NOs: 298, 3, 299, 306, 301 and 325;

SEQ ID NOs: 298, 3, 299, 306, 301 and 326;

SEQ ID NOs: 28, 29, 31, 33, 34 and 35;

SEQ ID NOs: 38, 39, 41, 43, 44 and 45;

SEQ ID NOs: 298, 3, 299, 306, 301 and 327;

SEQ ID NOs: 28, 29, 328, 50, 34 and 51;

SEQ ID NOs: 54, 55, 57, 59, 60 and 61;

SEQ ID NOs: 64, 65, 67, 59, 60 and 69;

SEQ ID NOs: 115, 72, 74, 76, 77 and 78;

SEQ ID NOs: 54, 55, 91, 93, 44 and 94;

SEQ ID NOs: 81, 82, 112, 86, 77 and 87;

SEQ ID NOs: 81, 82, 84, 86, 77 and 87;

SEQ ID NOs: 81, 82, 97, 86, 77 and 87;

SEQ ID NOs: 81, 82, 100, 86, 77 and 87;

SEQ ID NOs: 81, 82, 103, 86, 77 and 87;

SEQ ID NOs: 81, 82, 106, 86, 77 and 87; or

SEQ ID NOs: 81, 82, 109, 86, 77 and 87.

In various embodiments, the first antigen-binding site or first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively:

SEQ ID NOs: 81, 82, 112, 86, 77 and 87;

SEQ ID NOs: 81, 82, 84, 86, 77 and 87;

SEQ ID NOs: 81, 82, 97, 86, 77 and 87;

SEQ ID NOs: 81, 82, 100, 86, 77 and 87;

SEQ ID NOs: 81, 82, 103, 86, 77 and 87;

SEQ ID NOs: 81, 82, 106, 86, 77 and 87; or

SEQ ID NOs: 81, 82, 109, 86, 77 and 87.

In some embodiments, the first antigen-binding site or first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 81, 82, 112, 86, 77 and 87. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 81, 82, 84, 86, 77 and 87. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 81, 82, 97, 86, 77 and 87. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 81, 82, 100, 86, 77 and 87. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 81, 82, 103, 86, 77 and 87. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 81, 82, 106, 86, 77 and 87. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 81, 82, 109, 86, 77 and 87.

In various embodiments, the first antigen-binding site or first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Chothia numbering scheme), respectively:

SEQ ID NOs: 329, 330, 331, 332, 333 and 334;

SEQ ID NOs: 329, 330, 331, 335, 336 and 337;

SEQ ID NOs: 329, 330, 331, 338, 333 and 339;

SEQ ID NOs: 329, 330, 331, 338, 333 and 340;

SEQ ID NOs: 329, 330, 331, 332, 333 and 341;

SEQ ID NOs: 329, 330, 331, 551, 343 and 344;

SEQ ID NOs: 329, 330, 331, 332, 333 and 345;

SEQ ID NOs: 329, 330, 331, 332, 333 and 346;

SEQ ID NOs: 329, 330, 331, 332, 333 and 347;

SEQ ID NOs: 329, 330, 331, 332, 333 and 348;

SEQ ID NOs: 329, 330, 331, 332, 333 and 349;

SEQ ID NOs: 329, 330, 331, 332, 333 and 350;

SEQ ID NOs: 329, 330, 331, 332, 333 and 351;

SEQ ID NOs: 329, 330, 331, 332, 333 and 352;

SEQ ID NOs: 329, 330, 331, 332, 333 and 353;

SEQ ID NOs: 329, 330, 331, 332, 333 and 354;

SEQ ID NOs: 329, 330, 331, 332, 333 and 355;

SEQ ID NOs: 329, 330, 331, 338, 333 and 356;

SEQ ID NOs: 329, 330, 331, 338, 333 and 357;

SEQ ID NOs: 329, 330, 331, 332, 333 and 358;

SEQ ID NOs: 359, 360, 361, 362, 343 and 363;

SEQ ID NOs: 364, 365, 366, 367, 368 and 369;

SEQ ID NOs: 359, 360, 370, 371, 343 and 372;

SEQ ID NOs: 186, 373, 374, 375, 336 and 376;

SEQ ID NOs: 377, 378, 379, 375, 336 and 380;

SEQ ID NOs: 186, 373, 387, 388, 368 and 389;

SEQ ID NOs: 381, 390, 391, 392, 385 and 393;

SEQ ID NOs: 381, 390, 394, 392, 385 and 393;

SEQ ID NOs: 381, 390, 395, 392, 385 and 393;

SEQ ID NOs: 381, 390, 396, 392, 385 and 393;

SEQ ID NOs: 381, 390, 397, 392, 385 and 393;

SEQ ID NOs: 381, 390, 398, 392, 385 and 393; or

SEQ ID NOs: 381, 390, 399, 392, 385 and 393.

In various embodiments, the first antigen-binding site or first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Chothia numbering scheme), respectively:

SEQ ID NOs: 381, 390, 391, 392, 385 and 393;

SEQ ID NOs: 381, 390, 394, 392, 385 and 393;

SEQ ID NOs: 381, 390, 395, 392, 385 and 393;

SEQ ID NOs: 381, 390, 396, 392, 385 and 393;

SEQ ID NOs: 381, 390, 397, 392, 385 and 393;

SEQ ID NOs: 381, 390, 398, 392, 385 and 393; or

SEQ ID NOs: 381, 390, 399, 392, 385 and 393.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Chothia numbering scheme), respectively: SEQ ID NOs: 381, 390, 391, 392, 385 and 393. In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Chothia numbering scheme), respectively: SEQ ID NOs: 381, 390, 394, 392, 385 and 393. In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Chothia numbering scheme), respectively: SEQ ID NOs: 381, 390, 395, 392, 385 and 393. In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Chothia numbering scheme), respectively: SEQ ID NOs: 381, 390, 396, 392, 385 and 393. In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Chothia numbering scheme), respectively: SEQ ID NOs: 381, 390, 397, 392, 385 and 393. In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Chothia numbering scheme), respectively: SEQ ID NOs: 381, 390, 398, 392, 385 and 393. In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Chothia numbering scheme), respectively: SEQ ID NOs: 381, 390, 399, 392, 385 and 393.

In various embodiments, the first antigen-binding site or first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to IMGT numbering scheme), respectively:

SEQ ID NOs: 400, 401, 4, 402, 333 and 302;

SEQ ID NOs: 400, 401, 4, 552, 336 and 305;

SEQ ID NOs: 400, 401, 4, 403, 333 and 307;

SEQ ID NOs: 400, 401, 4, 403, 333 and 308;

SEQ ID NOs: 400, 401, 4, 402, 333 and 309;

SEQ ID NOs: 400, 401, 4, 405, 343 and 312;

SEQ ID NOs: 400, 401, 4, 402, 333 and 313;

SEQ ID NOs: 400, 401, 4, 402, 333 and 314;

SEQ ID NOs: 400, 401, 4, 402, 333 and 315;

SEQ ID NOs: 400, 401, 4, 403, 333 and 316;

SEQ ID NOs: 400, 401, 4, 402, 333 and 317;

SEQ ID NOs: 400, 401, 4, 402, 333 and 318;

SEQ ID NOs: 400, 401, 4, 402, 333 and 319;

SEQ ID NOs: 400, 401, 4, 402, 333 and 320;

SEQ ID NOs: 400, 401, 4, 402, 333 and 321;

SEQ ID NOs: 400, 401, 4, 402, 333 and 322;

SEQ ID NOs: 400, 401, 4, 402, 333 and 323;

SEQ ID NOs: 400, 401, 4, 403, 333 and 324;

SEQ ID NOs: 400, 401, 4, 403, 333 and 325;

SEQ ID NOs: 400, 401, 4, 402, 333 and 326;

SEQ ID NOs: 406, 407, 30, 408, 343 and 35;

SEQ ID NOs: 409, 410, 40, 411, 368 and 45;

SEQ ID NOs: 400, 401, 4, 402, 333 and 327;

SEQ ID NOs: 406, 407, 48, 412, 343 and 51;

SEQ ID NOs: 413, 414, 56, 415, 336 and 61;

SEQ ID NOs: 416, 417, 66, 415, 336 and 69;

SEQ ID NOs: 418, 419, 73, 420, 385 and 78;

SEQ ID NOs: 413, 414, 90, 421, 368 and 94;

SEQ ID NOs: 422, 423, 111, 424, 385 and 87;

SEQ ID NOs: 422, 423, 83, 424, 385 and 87;

SEQ ID NOs: 422, 423, 96, 424, 385 and 87;

SEQ ID NOs: 422, 423, 99, 424, 385 and 87;

SEQ ID NOs: 422, 423, 102, 424, 385 and 87;

SEQ ID NOs: 422, 423, 105, 424, 385 and 87; or

SEQ ID NOs: 422, 423, 108, 424, 385 and 87.

In various embodiments, the first antigen-binding site or first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to IMGT numbering scheme), respectively:

SEQ ID NOs: 422, 423, 111, 424, 385 and 87;

SEQ ID NOs: 422, 423, 83, 424, 385 and 87;

SEQ ID NOs: 422, 423, 96, 424, 385 and 87;

SEQ ID NOs: 422, 423, 99, 424, 385 and 87;

SEQ ID NOs: 422, 423, 102, 424, 385 and 87;

SEQ ID NOs: 422, 423, 105, 424, 385 and 87; or

SEQ ID NOs: 422, 423, 108, 424, 385 and 87.

In some embodiments, the first antigen-binding site or first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to IMGT numbering scheme), respectively: SEQ ID NOs: 422, 423, 111, 424, 385 and 87. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to IMGT numbering scheme), respectively: SEQ ID NOs: 422, 423, 83, 424, 385 and 87. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to IMGT numbering scheme), respectively: SEQ ID NOs: 422, 423, 96, 424, 385 and 87. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to IMGT numbering scheme), respectively: SEQ ID NOs: 422, 423, 99, 424, 385 and 87. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to IMGT numbering scheme), respectively: SEQ ID NOs: 422, 423, 102, 424, 385 and 87. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to IMGT numbering scheme), respectively: SEQ ID NOs: 422, 423, 105, 424, 385 and 87. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to IMGT numbering scheme), respectively: SEQ ID NOs: 422, 423, 108, 424, 385 and 87.

In various embodiments, the first antigen-binding site or first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Honegger numbering scheme), respectively:

SEQ ID NOs: 425, 426, 427, 428, 429 and 334;

SEQ ID NOs: 425, 426, 427, 430, 431 and 337;

SEQ ID NOs: 425, 426, 427, 432, 429 and 339;

SEQ ID NOs: 425, 426, 427, 432, 429 and 340;

SEQ ID NOs: 425, 426, 427, 428, 429 and 341;

SEQ ID NOs: 425, 426, 433, 434, 435 and 344;

SEQ ID NOs: 425, 426, 427, 428, 429 and 345;

SEQ ID NOs: 425, 426, 427, 428, 429 and 346;

SEQ ID NOs: 425, 426, 427, 428, 429 and 347;

SEQ ID NOs: 425, 426, 427, 432, 429 and 348;

SEQ ID NOs: 425, 426, 427, 428, 429 and 349;

SEQ ID NOs: 425, 426, 427, 428, 429 and 350;

SEQ ID NOs: 425, 426, 427, 428, 429 and 351;

SEQ ID NOs: 425, 426, 427, 428, 429 and 352;

SEQ ID NOs: 425, 426, 427, 428, 429 and 353;

SEQ ID NOs: 425, 426, 427, 428, 429 and 354;

SEQ ID NOs: 425, 426, 427, 428, 429 and 355;

SEQ ID NOs: 425, 426, 427, 432, 429 and 356;

SEQ ID NOs: 425, 426, 427, 432, 429 and 357;

SEQ ID NOs: 425, 426, 427, 428, 429 and 358;

SEQ ID NOs: 436, 437, 438, 439, 435 and 372;

SEQ ID NOs: 447, 448, 449, 450, 451 and 376;

SEQ ID NOs: 452, 453, 454, 450, 451 and 380;

SEQ ID NOs: 455, 456, 457, 458, 459 and 386;

SEQ ID NOs: 447, 448, 460, 461, 444 and 389;

SEQ ID NOs: 462, 463, 464, 465, 459 and 393;

SEQ ID NOs: 462, 463, 466, 465, 459 and 393;

SEQ ID NOs: 462, 463, 467, 465, 459 and 393;

SEQ ID NOs: 462, 463, 468, 465, 459 and 393;

SEQ ID NOs: 462, 463, 469, 465, 459 and 393;

SEQ ID NOs: 462, 463, 470, 465, 459 and 393; or

SEQ ID NOs: 462, 463, 471, 465, 459 and 393.

In various embodiments, the first antigen-binding site or first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Honegger numbering scheme), respectively:

SEQ ID NOs: 462, 463, 464, 465, 459 and 393;

SEQ ID NOs: 462, 463, 466, 465, 459 and 393;

SEQ ID NOs: 462, 463, 467, 465, 459 and 393;

SEQ ID NOs: 462, 463, 468, 465, 459 and 393;

SEQ ID NOs: 462, 463, 469, 465, 459 and 393;

SEQ ID NOs: 462, 463, 470, 465, 459 and 393; or

SEQ ID NOs: 462, 463, 471, 465, 459 and 393.

In some embodiments, the first antigen-binding site or first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Honegger numbering scheme), respectively: SEQ ID NOs: 462, 463, 464, 465, 459 and 393. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Honegger numbering scheme), respectively: SEQ ID NOs: 462, 463, 466, 465, 459 and 393. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Honegger numbering scheme), respectively: SEQ ID NOs: 462, 463, 467, 465, 459 and 393. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Honegger numbering scheme), respectively: SEQ ID NOs: 462, 463, 468, 465, 459 and 393. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Honegger numbering scheme), respectively: SEQ ID NOs: 462, 463, 469, 465, 459 and 393. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Honegger numbering scheme), respectively: SEQ ID NOs: 462, 463, 470, 465, 459 and 393. In some embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Honegger numbering scheme), respectively: SEQ ID NOs: 462, 463, 471, 465, 459 and 393.

In certain embodiments, the first antigen-binding site that binds NKG2D (e.g., human NKG2D) comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 1, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in Table 1. In certain embodiments, the first antigen-binding site comprises the heavy chain CDR1, CDR2, and CDR3, and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences of an antibody discloses in Table 1. In certain embodiments, the first antigen-binding site comprises the heavy chain CDR1, CDR2, and CDR3, and the light chain CDR1, CDR2, and CDR3 of an antibody disclosed in Tables 1, 1A, 1B, 1C or 1D. Sequence identity can be determined according to the BLAST algorithm (blast.ncbi.nlm.nih.gov/Blast.cgi), using default settings.

In certain embodiments, the first antigen-binding site that binds to NKG2D comprises a heavy chain variable domain derived from SEQ ID NO:1, such as by having an amino acid sequence at least 90% (e.g., at least 90%, at least 91%, at least 92%, at least 93%, at least 94%, at least 95%, at least 96%, at least 97%, at least 98%, at least 99%, or 100%) identical to SEQ ID NO:1, and/or incorporating amino acid sequences identical to the CDR1 (SEQ ID NO:2), CDR2 (SEQ ID NO:3), and CDR3 (SEQ ID NO:4) sequences of SEQ ID NO:1. The heavy chain variable domain related to SEQ ID NO:1 can be coupled with a variety of light chain variable domains to form an NKG2D binding site. For example, the first antigen-binding site that incorporates a heavy chain variable domain related to SEQ ID NO:1 can further incorporate a light chain variable domain selected from the sequences derived from SEQ ID NOs: 5, 6, 7, 8, 9, 12, 13, 14, 15, 16, 17, 18, 19, 20, 21, 22, 23, 24, 25, and 46. For example, the first antigen-binding site incorporates a heavy chain variable domain with amino acid sequences at least 90% (e.g., at least 90%, at least 91%, at least 92%, at least 93%, at least 94%, at least 95%, at least 96%, at least 97%, at least 98%, at least 99%, or 100%) identical to SEQ ID NO:1 and a light chain variable domain with amino acid sequences at least 90% (e.g., at least 90%, at least 91%, at least 92%, at least 93%, at least 94%, at least 95%, at least 96%, at least 97%, at least 98%, at least 99%, or 100%) identical to any one of the sequences selected from SEQ ID NOs: 5, 6, 7, 8, 9, 12, 13, 14, 15, 16, 17, 18, 19, 20, 21, 22, 23, 24, 25, and 46.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:10, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:11.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:26, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:32. In certain embodiments, the VH comprises CDR1, CDR2, and CDR3 comprising the amino acid sequences of SEQ ID NOs: 27 or 28, 29, and 30 or 31, respectively (e.g., SEQ ID NOs: 27, 29, and 30, respectively, or SEQ ID NOs: 28, 29, and 31, respectively). In certain embodiments, the VL comprises CDR1, CDR2, and CDR3 comprising the amino acid sequences of SEQ ID NOs: 33, 34, and 35, respectively. In certain embodiments, the first antigen-binding site comprises (a) a VH that comprises CDR1, CDR2, and CDR3 comprising the amino acid sequences of SEQ ID NOs: 27 or 28, 29, and 30 or 31, respectively (e.g., SEQ ID NOs: 27, 29, and 30, respectively, or SEQ ID NOs: 28, 29, and 31, respectively); and (b) a VL that comprises CDR1, CDR2, and CDR3 comprising the amino acid sequences of SEQ ID NOs: 33, 34, and 35, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:36, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 37 or 38, 39, and 40 or 41, respectively (e.g., SEQ ID NOs: 37, 39, and 40, respectively, or SEQ ID NOs: 38, 39, and 41, respectively). In certain embodiments, the VL comprises CDR1, CDR2, and CDR3 comprising the amino acid sequences of SEQ ID NOs: 43, 44, and 45, respectively. In certain embodiments, the first antigen-binding site comprises (a) a VH that comprises CDR1, CDR2, and CDR3 comprising the amino acid sequences of SEQ ID NOs: 37 or 38, 39, and 40 or 41, respectively (e.g., SEQ ID NOs: 37, 39, and 40, respectively, or SEQ ID NOs: 38, 39, and 41, respectively); and (b) a VL that comprises CDR1, CDR2, and CDR3 comprising the amino acid sequences of SEQ ID NOs: 43, 44, and 45, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:47, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:49. In certain embodiments, the VH comprises CDR1, CDR2, and CDR3 comprising the amino acid sequences of SEQ ID NOs: 27, 29, and 48, respectively. In certain embodiments, the VL comprises CDR1, CDR2, and CDR3 comprising the amino acid sequences of SEQ ID NOs: 50, 34, and 51, respectively. In certain embodiments, the first antigen-binding site comprises (a) a VH that comprises CDR1, CDR2, and CDR3 comprising the amino acid sequences of SEQ ID NOs: 27, 29, and 48, respectively; and (b) a VL that comprises CDR1, CDR2, and CDR3 comprising the amino acid sequences of SEQ ID NOs: 50, 34, and 51, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:52, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:58. In certain embodiments, the VH comprises CDR1, CDR2, and CDR3 comprising the amino acid sequences of SEQ ID NOs: 53 or 54, 55, and 56 or 57, respectively (e.g., SEQ ID NOs: 53, 55, and 56, respectively, or SEQ ID NOs: 54, 55, and 57, respectively). In certain embodiments, the VL comprises CDR1, CDR2, and CDR3 comprising the amino acid sequences of SEQ ID NOs: 59, 60, and 61, respectively. In certain embodiments, the first antigen-binding site comprises (a) a VH that comprises CDR1, CDR2, and CDR3 comprising the amino acid sequences of SEQ ID NOs: 53 or 54, 55, and 56 or 57, respectively (e.g., SEQ ID NOs: 53, 55, and 56, respectively, or SEQ ID NOs: 54, 55, and 57, respectively); and (b) a VL that comprises CDR1, CDR2, and CDR3 comprising the amino acid sequences of SEQ ID NOs: 59, 60, and 61, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:62, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:68. In certain embodiments, the VH comprises CDR1, CDR2, and CDR3 comprising the amino acid sequences of SEQ ID NOs: 63 or 64, 65, and 66 or 67, respectively (e.g., SEQ ID NOs: 63, 65, and 66, respectively, or SEQ ID NOs: 64, 65, and 67, respectively). In certain embodiments, the VL comprises CDR1, CDR2, and CDR3 comprising the amino acid sequences of SEQ ID NOs: 59, 60, and 69, respectively. In certain embodiments, the first antigen-binding site comprises (a) a VH that comprises CDR1, CDR2, and CDR3 comprising the amino acid sequences of SEQ ID NOs: 63 or 64, 65, and 66 or 67, respectively (e.g., SEQ ID NOs: 63, 65, and 66, respectively, or SEQ ID NOs: 64, 65, and 67, respectively); and (b) a VL that comprises CDR1, CDR2, and CDR3 comprising the amino acid sequences of SEQ ID NOs: 59, 60, and 69, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:89, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:92. In certain embodiments, the VH comprises CDR1, CDR2, and CDR3 comprising the amino acid sequences of SEQ ID NOs: 53 or 54, 55, and 90 or 91, respectively (e.g., SEQ ID NOs: 53, 55, and 90, respectively, or SEQ ID NOs: 54, 55, and 91, respectively). In certain embodiments, the VL comprises CDR1, CDR2, and CDR3 comprising the amino acid sequences of SEQ ID NOs: 93, 44, and 94, respectively. In certain embodiments, the first antigen-binding site comprises (a) a VH that comprises CDR1, CDR2, and CDR3 comprising the amino acid sequences of SEQ ID NOs: 53 or 54, 55, and 90 or 91, respectively (e.g., SEQ ID NOs: 53, 55, and 90, respectively, or SEQ ID NOs: 54, 55, and 91, respectively); and (b) a VL that comprises CDR1, CDR2, and CDR3 comprising the amino acid sequences of SEQ ID NOs: 93, 44, and 94, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:70, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:75. In certain embodiments, the VH comprises CDR1, CDR2, and CDR3 comprising the amino acid sequences of SEQ ID NOs: 71 or 115, 72, and 73 or 74, respectively (e.g., SEQ ID NOs: 71, 72, and 73, respectively, or SEQ ID NOs: 115, 72, and 74, respectively). In certain embodiments, the VL comprises CDR1, CDR2, and CDR3 comprising the amino acid sequences of SEQ ID NOs: 76, 77, and 78, respectively. In certain embodiments, the first antigen-binding site comprises (a) a VH that comprises CDR1, CDR2, and CDR3 comprising the amino acid sequences of SEQ ID NOs: 71 or 115, 72, and 73 or 74, respectively (e.g., SEQ ID NOs: 71, 72, and 73, respectively, or SEQ ID NOs: 115, 72, and 74, respectively); and (b) a VL that comprises CDR1, CDR2, and CDR3 comprising the amino acid sequences of SEQ ID NOs: 76, 77, and 78, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:79, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 83 or 84, respectively (e.g., SEQ ID NOs: 80, 82, and 83, respectively, or SEQ ID NOs: 81, 82, and 84, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 83 or 84 respectively (e.g., SEQ ID NOs: 80, 82, and 83, respectively, or SEQ ID NOs: 81, 82, and 84, respectively); and (b) a VL that comprises CDR1, CDR2, and CDR3 comprising the amino acid sequences of SEQ ID NOs: 86, 77, and 87, respectively. In certain embodiments, the first antigen-binding site that binds NKG2D comprises an scFv with a Q44C substitution in VH and G100C substitution in VL. Accordingly, in some embodiments, the first antigen-binding site that binds NKG2D comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:88.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:95, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 96 or 97, respectively (e.g., SEQ ID NOs: 80, 82, and 96, respectively, or SEQ ID NOs: 81, 82, and 97, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 96 or 97, respectively (e.g., SEQ ID NOs: 80, 82, and 96, respectively, or SEQ ID NOs: 81, 82, and 97, respectively); and (b) a VL that comprises CDR1, CDR2, and CDR3 comprising the amino acid sequences of SEQ ID NOs: 86, 77, and 87, respectively. In certain embodiments, the first antigen-binding site that binds NKG2D comprises an scFv with a Q44C substitution in VH and G100C substitution in VL. Accordingly, in some embodiments, the first antigen-binding site that binds NKG2D comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:288.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:98, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 99 or 100, respectively (e.g., SEQ ID NOs: 80, 82, and 99, respectively, or SEQ ID NOs: 81, 82, and 100, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 99 or 100, respectively (e.g., SEQ ID NOs: 80, 82, and 99, respectively, or SEQ ID NOs: 81, 82, and 100, respectively); and (b) a VL that comprises CDR1, CDR2, and CDR3 comprising the amino acid sequences of SEQ ID NOs: 86, 77, and 87, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:101, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 102 or 103, respectively (e.g., SEQ ID NOs: 80, 82, and 102, respectively, or SEQ ID NOs: 81, 82, and 103, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 102 or 103, respectively (e.g., SEQ ID NOs: 80, 82, and 102, respectively, or SEQ ID NOs: 81, 82, and 103, respectively); and (b) a VL that comprises CDR1, CDR2, and CDR3 comprising the amino acid sequences of SEQ ID NOs: 86, 77, and 87, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:104, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 105 or 106, respectively (e.g., SEQ ID NOs: 80, 82, and 105, respectively, or SEQ ID NOs: 81, 82, and 106, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 105 or 106, respectively (e.g., SEQ ID NOs: 80, 82, and 105, respectively, or SEQ ID NOs: 81, 82, and 106, respectively); and (b) a VL that comprises CDR1, CDR2, and CDR3 comprising the amino acid sequences of SEQ ID NOs: 86, 77, and 87, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:107, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 108 or 109, respectively (e.g., SEQ ID NOs: 80, 82, and 108, respectively, or SEQ ID NOs: 81, 82, and 109, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 108 or 109, respectively (e.g., SEQ ID NOs: 80, 82, and 108, respectively, or SEQ ID NOs: 81, 82, and 109, respectively); and (b) a VL that comprises CDR1, CDR2, and CDR3 comprising the amino acid sequences of SEQ ID NOs: 86, 77, and 87, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:110, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 111 or 112, respectively (e.g., SEQ ID NOs: 80, 82, and 111, respectively, or SEQ ID NOs: 81, 82, and 112, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 111 or 112, respectively (e.g., SEQ ID NOs: 80, 82, and 111, respectively, or SEQ ID NOs: 81, 82, and 112, respectively); and (b) a VL that comprises CDR1, CDR2, and CDR3 comprising the amino acid sequences of SEQ ID NOs: 86, 77, and 87, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:113, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:114.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:116, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:117.

The multispecific binding proteins can bind to NKG2D-expressing cells, which include but are not limited to NK cells, 76 T cells and CD8+ap T cells. Upon NKG2D binding, the multispecific binding proteins may block natural ligands, such as ULBP6 and MICA, from binding to NKG2D and activating NK cells.

The multispecific binding proteins binds to cells expressing CD16, an Fc receptor on the surface of leukocytes including natural killer cells, macrophages, neutrophils, eosinophils, mast cells, and follicular dendritic cells.

In certain embodiments, a multispecific binding protein of the present disclosure specifically binds to NKG2D (e.g., human NKG2D) with an affinity of K_{D} (i.e., dissociation constant) of 2 nM to 400 nM, e.g., 2 nM to 390 nM, 2 nM to 390 nM, 2 nM to 380 nM, 2 nM to 370 nM, 2 nM to 360 nM, 2 nM to 350 nM, 2 nM to 340 nM, 2 nM to 330 nM, 2 nM to 320 nM, 2 nM to 310 nM, 2 nM to 300 nM, 2 nM to 290 nM, 2 nM to 280 nM, 2 nM to 270 nM, 2 nM to 260 nM, 2 nM to 250 nM, 2 nM to 240 nM, 2 nM to 230 nM, 2 nM to 220 nM, 2 nM to 210 nM, 2 nM to 200 nM, 2 nM to 190 nM, 2 nM to 180 nM, 2 nM to 170 nM, 2 nM to 160 nM, 2 nM to 150 nM, 2 nM to 140 nM, 2 nM to 130 nM, 2 nM to 120 nM, 2 nM to 110 nM, 2 nM to 100 nM, 2 nM to 90 nM, 2 nM to 80 nM, 2 nM to 70 nM, 2 nM to 60 nM, 2 nM to 50 nM, 2 nM to 40 nM, 2 nM to 30 nM, 2 nM to 20 nM, 2 nM to 10 nM, 5 nM to 400 nM, 10 nM to 400 nM, 20 nM to 400 nM, 30 nM to 400 nM, 40 nM to 400 nM, 50 nM to 400 nM, 60 nM to 400 nM, 70 nM to 400 nM, 80 nM to 400 nM, 90 nM to 400 nM, 100 nM to 400 nM, 110 nM to 400 nM, 120 nM to 400 nM, 130 nM to 400 nM, 140 nM to 400 nM, 150 nM to 400 nM, 160 nM to 400 nM, 170 nM to 400 nM, 180 nM to 400 nM, 190 nM to 400 nM, 200 nM to 400 nM, 210 nM to 400 nM, 220 nM to 400 nM, 230 nM to 400 nM, 240 nM to 400 nM, 250 nM to 400 nM, 260 nM to 400 nM, 270 nM to 400 nM, 280 nM to 400 nM, 290 nM to 400 nM, 300 nM to 400 nM, 310 nM to 400 nM, 320 nM to 400 nM, 330 nM to 400 nM, 340 nM to 400 nM, 350 nM to 400 nM, 360 nM to 400 nM, 370 nM to 400 nM, 380 nM to 400 nM, 390 nM to 400 nM, 100 nM to 380 nM, 200 nM to 380 nM, 300 nM to 380 nM, 350 nM to 380 nM, 360 nM to 380 nM, 100 nM to 200 nM, 200 nM to 300 nM, about 400 nM, about 390 nM, about 380 nM, about 370 nM, about 360 nM, about 350 nM, about 340 nM, about 330 nM, about 310 nM, about 300 nM, about 290 nM, about 280 nM, about 270 nM, about 260 nM, about 250 nM, about 240 nM, about 230 nM, about 220 nM, about 210 nM, about 200 nM, about 190 nM, about 180 nM, about 170 nM, about 160 nM, about 150 nM, about 140 nM, about 130 nM, about 120 nM, about 110 nM, about 100 nM, about 90 nM, about 80 nM, about 70 nM, about 60 nM, about 50 nM, about 40 nM, about 30 nM, about 20 nM, about 15 nM, about 14 nM, about 13 nM, about 12 nM, about 11 nM, about 10 nM, about 9 nM, about 8 nM, about 7 nM, about 6 nM, about 5 nM, about 4.5 nM, about 4 nM, about 3.5 nM, about 3 nM, about 2.5 nM, about 2 nM, about 1.5 nM, about 1 nM, between about 0.5 nM to about 1 nM, about 1 nM to about 2 nM, about 2 nM to 3 nM, about 3 nM to 4 nM, about 4 nM to about 5 nM, about 5 nM to about 6 nM, about 6 nM to about 7 nM, about 7 nM to about 8 nM, about 8 nM to about 9 nM, about 9 nM to about 10 nM, about 1 nM to about 10 nM, about 2 nM to about 10 nM, about 3 nM to about 10 nM, about 4 nM to about 10 nM, about 5 nM to about 10 nM, about 6 nM to about 10 nM, about 7 nM to about 10 nM, about 8 nM to about 10 nM, about 100 nM to about 400 nM, about 200 nM to about 400 nM, or about 300 nM to about 400 nM as measured using standard binding assays, for example, surface plasmon resonance (SPR) (e.g., using the method described in Example 1 infra) or bio-layer interferometry (BLI). In some embodiments, NKG2D-binding sites specifically bind to NKG2D with a K_{D} of 10 to 62 nM. In some embodiments, NKG2D-binding sites specifically bind to NKG2D with a K_{D} of 300 to 400 nM. In some embodiments, NKG2D-binding sites specifically bind to NKG2D with a K_{D} of 360 to 380 nM. In certain embodiments, a multispecific binding protein of the present disclosure specifically binds NKG2D (e.g., human NKG2D) with a K_{d} (i.e., off-rate, also called K_{off}) equal to or lower than 1×10⁻⁵, 1×10⁻⁴, 1×10⁻³, 5×10⁻³, 0.01, 0.02, 0.05, 0.06, 0.07, 0.08, 0.09, 0.10, 0.2, 0.3, 0.4, or 0.5 l/s, as measured by SPR (e.g., using the method described in Example 1 infra) or by VLL.

### 5T4 Binding Site or 5T4 Binding Domain

The 5T4 site of the multispecific binding protein disclosed herein comprises a heavy chain variable domain and a light chain variable domain.

In one aspect, the present disclosure provides multispecific binding proteins that bind to the NKG2D receptor and CD16 receptor on natural killer cells, and 5T4. Table 2 lists some exemplary sequences of heavy chain variable domains and light chain variable domains that, in combination, can bind to 5T4.

CDR sequences in Table 2 are identified under Chothia numbering unless otherwise indicated. Table 2A provides CDR sequences according to Kabat numbering scheme. Table 2B provides CDR sequences according to Chothia numbering scheme. Table 2C provides CDR sequences according to IMGT numbering scheme. Table 2D provides CDR sequences according to Honegger numbering scheme.

TABLE 2
Sequences of Exemplary Antigen-Binding Sites
or Antigen-Binding Domains that AB 1Bind 5T4

| Clone | VH | VL |
|---|---|---|
| 10F10 murine | EVQLQESGGGLVKPGGSRRLSCAA | DIVLTQSPASLAVTLGQRATISCR |
| | SRFTFSDFGMHWVRQAPEKGLEWI | ASQSVTTSNYNYMHWFHQKPGQPP |
| | AYISSGSSTIYYADTVKGRFTISR | KLLIKFASNLESGVPARFSGSGSG |
| | DNAKNTLFLQMTSLRSEDTAMYYC | TDFTLNIHPVEEEDIATYYCQHSW |
| | ASSQSYYRGTLDYWGQGTSVTVSS | EIPWTFGGGTKLEIK |
| | (SEQ ID NO: 136) | (SEQ ID NO: 137) |
| | CDR1-RFTFSDF | CDR1-RASQSVTTSNYNYMH |
| | (SEQ ID NO: 138) | (SEQ ID NO: 141) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| AB1002 | EVQLVESGGGLVKPGGSLRLSCAA | DIQLTQSPSSLSASVGDRVTITCR |
| (humanized | SRFTFSDFGMHWVRQAPGKGLEWV | ASQSVTTSNYNYMHWFQQKPGKAP |
| 10F10, T62S) | SYISSGSSTIYYADSVKGRFTISR | KLLIKFASNLESGVPSRFSGSGSG |
| | DNAKNSLYLQMNSLRAEDTAVYYC | TDFTLTISSLQPEDFATYYCQHSW |
| | ASSQSYYRGTLDYWGQGTTVTVSS | EIPWTFGGGTKVEIK |
| | (SEQ ID NO: 144) | (SEQ ID NO: 145) |
| | CDR1-RFTFSDF | CDR1-RASQSVTTSNYNYMH |
| | (SEQ ID NO: 138) | (SEQ ID NO: 141) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| AB1002 (with | EVQLVESGGGLVKPGGSLRLSCAA | DIQLTQSPSSLSASVGDRVTITCR |
| cysteine | SRFTFSDFGMHWVRQAPGKCLEWV | ASQSVTTSNYNYMHWFQQKPGKAP |
| heterodimerization | SYISSGSSTIYYADSVKGRFTISR | KLLIKFASNLESGVPSRFSGSGSG |
| mutations for | DNAKNSLYLQMNSLRAEDTAVYYC | TDFTLTISSLQPEDFATYYCQHSW |
| disulfide bond | ASSQSYYRGTLDYWGQGTTVTVSS | EIPWTFGCGTKVEIK |
| formation) | (SEQ ID NO: 146) | (SEQ ID NO: 147) |
| | CDR1-RFTFSDF | CDR1-RASQSVTTSNYNYMH |
| | (SEQ ID NO: 138) | (SEQ ID NO: 141) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| AB1002 scFv | DIQLTQSPSSLSASVGDRVTITCRASQSVTTSNYNYMHWFQQKPGKAPK | |
| (VL-VH) | LLIKFASNLESGVPSRFSGSGSGTDFTLTISSLQPEDFATYYCQHSWEI | |
| (AB1310 | PWTFGCGTKVEIKGGGGSGGGGSGGGGSGGGGSEVQLVESGGGLVKPGG | |
| TriNKET ®) | SLRLSCAASRFTFSDFGMHWVRQAPGKCLEWVSYISSGSSTIYYADSVK | |
| | GRFTISRDNAKNSLYLQMNSLRAEDTAVYYCASSQSYYRGTLDYWGQGT | |
| | TVTVSS (SEQ ID NO: 148) | |
| AB1002 scFv | EVQLVESGGGLVKPGGSLRLSCAASRFTFSDFGMHWVRQAPGKCLEWVS | |
| (VH-VL) | YISSGSSTIYYADSVKGRFTISRDNAKNSLYLQMNSLRAEDTAVYYCAS | |
| | SQSYYRGTLDYWGQGTTVTVSSGGGGSGGGGSGGGGSGGGGSDIQLTQS | |
| | PSSLSASVGDRVTITCRASQSVTTSNYNYMHWFQQKPGKAPKLLIKFAS | |
| | NLESGVPSRFSGSGSGTDFTLTISSLQPEDFATYYCQHSWEIPWTFGCG | |
| | TKVEIK (SEQ ID NO: 149) | |
| AB1002 | EVQLVESGGGLVKPGGSLRLSCAA | DIQLTQSPSSLSASVGDRVTITCR |
| (humanized | SRFTFSDFGMHWVRQAPGKCLEWV | ASQSVTTSNYNYLHWFQQKPGKAP |
| 10F10) M33L | SYISSGSSTIYYADSVKGRFTISR | KLLIKFASNLESGVPSRFSGSGSG |
| | DNAKNSLYLQMNSLRAEDTAVYYC | TDFTLTISSLQPEDFATYYCQHSW |
| | ASSQSYYRGTLDYWGQGTTVTVSS | EIPWTFGGGTKVEIK |
| | (SEQ ID NO: 144) | (SEQ ID NO: 289) |
| | CDR1-RFTFSDF | CDR1-RASQSVTTSNYNYLH |
| | (SEQ ID NO: 138) | (SEQ ID NO: 290) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| AB1002 | EVQLVESGGGLVKPGGSLRLSCAA | DIQLTQSPSSLSASVGDRVTITCR |
| (humanized | SRFTFSDFGMHWVRQAPGKGLEWV | ASQSVTTSNYNYVHWFQQKPGKAP |
| 10F10) M33V | SYISSGSSTIYYADSVKGRFTISR | KLLIKFASNLESGVPSRFSGSGSG |
| | DNAKNSLYLQMNSLRAEDTAVYYC | TDFTLTISSLQPEDFATYYCQHSW |
| | ASSQSYYRGTLDYWGQGTTVTVSS | EIPWTFGGGTKVEIK |
| | (SEQ ID NO: 144) | (SEQ ID NO: 291) |
| | CDR1-RFTFSDF | CDR1-RASQSVTTSNYNYVH |
| | (SEQ ID NO: 138) | (SEQ ID NO: 292) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| 05H04 | QVQLVQSGAEVKKPGASVKVSCKA | ENVLTQSPATLSLSPGERATLSCS |
| | SGYKFTDYYMDWVRQAPGQGLEWI | AKSSVSYIHWYQQKPGQAPRLLIY |
| | GYIFPNDASTTYNEKFKGKATLTA | DTSYLGSGIPARFSGSGSGTDYTL |
| | DKSTNTAYMELSSLRSEDTAVYYC | TISSLEPEDFAVYYCQQWSSYPYT |
| | ARSRDADYWGQGTTVTVSS | FGGGTKVEIK |
| | (SEQ ID NO: 150) | (SEQ ID NO: 151) |
| | CDR1-GYKFTDY | CDR1-SAKSSVSYIH |
| | (SEQ ID NO: 152) | (SEQ ID NO: 154) |
| | CDR2-FPNDAS | CDR2-DTSYLGS |
| | (SEQ ID NO: 158) | (SEQ ID NO: 155) |
| | CDR3-SRDADY | CDR3-QQWSSYPYT |
| | (SEQ ID NO: 153) | (SEQ ID NO: 156) |
| 05H04 Q1E | EVQLVQSGAEVKKPGASVKVSCKA | ENVLTQSPATLSLSPGERATLSCS |
| | SGYKFTDYYMDWVRQAPGQGLEWI | AKSSVSYIHWYQQKPGQAPRLLIY |
| | GYIFPNDASTTYNEKFKGKATLTA | DTSYLGSGIPARFSGSGSGTDYTL |
| | DKSTNTAYMELSSLRSEDTAVYYC | TISSLEPEDFAVYYCQQWSSYPYT |
| | ARSRDADYWGQGTTVTVSS (SEQ | FGGGTKVEIK |
| | (SEQ ID NO: 157) | (SEQ ID NO: 151) |
| | CDR1-GYKFTDY | CDR1-SAKSSVSYIH |
| | (SEQ ID NO: 152) | (SEQ ID NO: 154) |
| | CDR2-FPNDAS | CDR2-DTSYLGS |
| | (SEQ ID NO: 158) | (SEQ ID NO: 155) |
| | CDR3-SRDADY | CDR3-QQWSSYPYT |
| | (SEQ ID NO: 153) | (SEQ ID NO: 156) |
| 05H04 scFv | QVQLVQSGAEVKKPGASVKVSCKASGYKFTDYYMDWVRQAPGQCLEWIG | |
| (VH-VL) | YIFPNDASTTYNEKFKGKATLTADKSTNTAYMELSSLRSEDTAVYYCAR | |
| | SRDADYWGQGTTVTVSSGGGGSGGGGSGGGGSGGGGSENVLTQSPATLS | |
| | LSPGERATLSCSAKSSVSYIHWYQQKPGQAPRLLIYDTSYLGSGIPARF | |
| | SGSGSGTDYTLTISSLEPEDFAVYYCQQWSSYPYTFGCGTKVEIK | |
| | (SEQ ID NO: 159) | |
| 05H04 scFv | ENVLTQSPATLSLSPGERATLSCSAKSSVSYIHWYQQKPGQAPRLLIYD | |
| (VL-VH) | TSYLGSGIPARFSGSGSGTDYTLTISSLEPEDFAVYYCQQWSSYPYTFG | |
| | CGTKVEIKGGGGSGGGGSGGGGSGGGGSQVQLVQSGAEVKKPGASVKVS | |
| | CKASGYKFTDYYMDWVRQAPGQCLEWIGYIFPNDASTTYNEKFKGKATL | |
| | TADKSTNTAYMELSSLRSEDTAVYYCARSRDADYWGQGTTVTVSS | |
| | (SEQ ID NO: 221) | |
| 05H04 Q1E | EVQLVQSGAEVKKPGASVKVSCKASGYKFTDYYMDWVRQAPGQCLEWIG | |
| (VH-VL) | YIFPNDASTTYNEKFKGKATLTADKSTNTAYMELSSLRSEDTAVYYCAR | |
| | SRDADYWGQGTTVTVSSGGGGSGGGGSGGGGSGGGGSENVLTQSPATLS | |
| | LSPGERATLSCSAKSSVSYIHWYQQKPGQAPRLLIYDTSYLGSGIPARF | |
| | SGSGSGTDYTLTISSLEPEDFAVYYCQQWSSYPYTFGCGTKVEIK | |
| | (SEQ ID NO: 160) | |
| 11F09 | EVQLVESGGGLVKPGGSLRLSCEA | DIQLTQSPSSLSASVGDRVTITCR |
| | SGFTFSDYGMHWVRQAPGKGLEWL | ASQGVSTSTYTYLHWFQQKPGKAP |
| | AYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 161) | (SEQ ID NO: 162) |
| | CDR1-GFTFSDY | CDR1-RASQGVSTSTYTYLH |
| | (SEQ ID NO: 163) | (SEQ ID NO: 165) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| 10F10 21*05 | EVQLVESGGGLVKPGGSLRLSCAA | DIQLTQSPSSLSASVGDRVTITCR |
| AB1002 parental | SRFTFSDFGMHWVRQAPGKGLEWV | ASQSVTTSNYNYMHWFQQKPGKAP |
| humanized (T62) | SYISSGSSTIYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | ASSQSYYRGTLDYWGQGTTVTVSS | EIPWTFGGGTKVEIK |
| | (SEQ ID NO: 166) | (SEQ ID NO: 145) |
| | CDR1-RFTFSDF | CDR1-RASQSVTTSNYNYMH |
| | (SEQ ID NO: 138) | (SEQ ID NO: 141) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| 10F10 21*05 scFv | DIQLTQSPSSLSASVGDRVTITCRASQSVTTSNYNYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCAASRFTFSDFGMHWVRQAPGKCLEWVSYISSGSSTIYYADTVK | |
| | GRETISRDNAKNSLYLQMNSLRAEDTAVYYCASSQSYYRGTLDYWGQGT | |
| | TVTVSS (SEQ ID NO: 167) | |
| 10F10 23*03 | EVQLLESGGGLVQPGGSLRLSCAA | DIQLTQSPSSLSASVGDRVTITCR |
| humanized variant | SRFTFSDFGMHWVRQAPGKGLEWI | ASQSVTTSNYNYMHWFQQKPGKAP |
| 2 | AYISSGSSTIYYADTVKGRFTISR | KLLIKFASNLESGVPSRFSGSGSG |
| | DNAKNTLYLQMNSLRAEDTAVYYC | TDFTLTISSLQPEDFATYYCQHSW |
| | ASSQSYYRGTLDYWGQGTTVTVSS | EIPWTFGGGTKVEIK |
| | (SEQ ID NO: 168) | (SEQ ID NO: 145) |
| | CDR1-RFTFSDF | CDR1-RASQSVTTSNYNYMH |
| | (SEQ ID NO: 138) | (SEQ ID NO: 141) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| 10F10 23*03 scFv | DIQLTQSPSSLSASVGDRVTITCRASQSVTTSNYNYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLLESGGGLVQPGG | |
| | SLRLSCAASRFTFSDFGMHWVRQAPGKCLEWIAYISSGSSTIYYADTVK | |
| | GRFTISRDNAKNTLYLQMNSLRAEDTAVYYCASSQSYYRGTLDYWGQGT | |
| | TVTVSS (SEQ ID NO: 169) | |
| 10F10 23*03 BM1 | EVQLLESGGGLVQPGGSLRLSCAA | DIQLTQSPSSLSASVGDRVTITCR |
| | SRFTFSDFGMHWVRQAPGKGLEWV | ASQSVTTSNYNYMHWFQQKPGKAP |
| | SYISSGSSTIYYADTVKGRFTISR | KLLIKFASNLESGVPSRFSGSGSG |
| | DNSKNTLYLQMNSLRAEDTAVYYC | TDFTLTISSLQPEDFATYYCQHSW |
| | ASSQSYYRGTLDYWGQGTTVTVSS | EIPWTFGGGTKVEIK |
| | (SEQ ID NO: 236) | (SEQ ID NO: 145) |
| | CDR1-RFTFSDF | CDR1-RASQSVTTSNYNYMH |
| | (SEQ ID NO: 138) | (SEQ ID NO: 141) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| 10F10 23*03 BM1 | DIQLTQSPSSLSASVGDRVTITCRASQSVTTSNYNYMHWFQQKPGKAPK | |
| scFv (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLLESGGGLVQPGG | |
| | SLRLSCAASRFTFSDFGMHWVRQAPGKCLEWVSYISSGSSTIYYADTVK | |
| | GRFTISRDNSKNTLYLQMNSLRAEDTAVYYCASSQSYYRGTLDYWGQGT | |
| | TVTVSS (SEQ ID NO: 293) | |
| 10F10 48*01 | EVQLVESGGGLVQPGGSLRLSCAA | DIQLTQSPSSLSASVGDRVTITCR |
| humanized variant | SRFTFSDFGMHWVRQAPGKGLEWV | ASQSVTTSNYNYMHWFQQKPGKAP |
| 3 | SYISSGSSTIYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | ASSQSYYRGTLDYWGQGTTVTVSS | EIPWTFGGGTKVEIK |
| | (SEQ ID NO: 170) | (SEQ ID NO: 145) |
| | CDR1-RFTFSDF | CDR1-RASQSVTTSNYNYMH |
| | (SEQ ID NO: 138) | (SEQ ID NO: 141) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| 10F10 48*01 scFv | DIQLTQSPSSLSASVGDRVTITCRASQSVTTSNYNYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVQPGG | |
| | SLRLSCAASRFTFSDFGMHWVRQAPGKCLEWVSYISSGSSTIYYADTVK | |
| | GRFTISRDNAKNSLYLQMNSLRAEDTAVYYCASSQSYYRGTLDYWGQGT | |
| | TVTVSS (SEQ ID NO: 171) | |
| 10F10 48*01 BM2 | EVQLVESGGGLVQPGGSLRLSCAA | DIQLTQSPSSLSASVGDRVTITCR |
| | SRFTFSDFGMHWVRQAPGKGLEWI | ASQSVTTSNYNYMHWFQQKPGKAP |
| | AYISSGSSTIYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | ASSQSYYRGTLDYWGQGTTVTVSS | EIPWTFGGGTKVEIK |
| | (SEQ ID NO: 228) | (SEQ ID NO: 145) |
| | CDR1-RFTFSDF | CDR1-RASQSVTTSNYNYMH |
| | (SEQ ID NO: 138) | (SEQ ID NO: 141) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| 10F10 48*01 BM2 | DIQLTQSPSSLSASVGDRVTITCRASQSVTTSNYNYMHWFQQKPGKAPK | |
| scFv (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVQPGG | |
| | SLRLSCAASRFTFSDFGMHWVRQAPGKCLEWIAYISSGSSTIYYADTVK | |
| | GRFTISRDNAKNSLYLQMNSLRAEDTAVYYCASSQSYYRGTLDYWGQGT | |
| | TVTVSS (SEQ ID NO: 229) | |
| 10F10 11*01 | QVQLVESGGGLVKPGGSLRLSCAA | DIQLTQSPSSLSASVGDRVTITCR |
| humanized variant | SRFTFSDFGMHWVRQAPGKGLEWI | ASQSVTTSNYNYMHWFQQKPGKAP |
| 4 | AYISSGSSTIYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | ASSQSYYRGTLDYWGQGTTVTVSS | EIPWTFGGGTKVEIK |
| | (SEQ ID NO: 172) | (SEQ ID NO: 145) |
| | CDR1-RFTFSDF | CDR1-RASQSVTTSNYNYMH |
| | (SEQ ID NO: 138) | (SEQ ID NO: 141) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| 10F10 11*01 scFv | DIQLTQSPSSLSASVGDRVTITCRASQSVTTSNYNYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSQVQLVESGGGLVKPGG | |
| | SLRLSCAASRFTFSDFGMHWVRQAPGKCLEWIAYISSGSSTIYYADTVK | |
| | GRFTISRDNAKNSLYLQMNSLRAEDTAVYYCASSQSYYRGTLDYWGQGT | |
| | TVTVSS (SEQ ID NO: 173) | |
| 10F10 21*05 | EVQLVESGGGLVKPGGSLRLSCAA | DIQLTQSPSSLSASVGDRVTITCR |
| humanized variant | SRFTFSDFGMHWVRQAPGKGLEWI | ASQSVTTSNYNYMHWFQQKPGKAP |
| 5 | AYISSGSSTIYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | ASSQSYYRGTLDYWGQGTTVTVSS | EIPWTFGGGTKVEIK |
| | (SEQ ID NO: 174) | (SEQ ID NO: 145) |
| | CDR1-RFTFSDF | CDR1-RASQSVTTSNYNYMH |
| | (SEQ ID NO: 138) | (SEQ ID NO: 141) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| 10F10 21*05 scFv | DIQLTQSPSSLSASVGDRVTITCRASQSVTTSNYNYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCAASRFTFSDFGMHWVRQAPGKCLEWIAYISSGSSTIYYADTVK | |
| | GRFTISRDNAKNSLYLQMNSLRAEDTAVYYCASSQSYYRGTLDYWGQGT | |
| | TVTVSS (SEQ ID NO: 175) | |
| 10F10 11*01 BM1 | EVQLVESGGGLVKPGGSLRLSCAA | DIQLTQSPSSLSASVGDRVTITCR |
| | SRFTFSDFGMHWIRQAPGKGLEWV | ASQSVTTSNYNYMHWFQQKPGKAP |
| | SYISSGSSTIYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | ASSQSYYRGTLDYWGQGTTVTVSS | EIPWTFGGGTKVEIK |
| | G (SEQ ID NO: 232) | (SEQ ID NO: 145) |
| | CDR1-RFTFSDF | CDR1-RASQSVTTSNYNYMH |
| | (SEQ ID NO: 138) | (SEQ ID NO: 141) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| 10F10 11*01 scFv | DIQLTQSPSSLSASVGDRVTITCRASQSVTTSNYNYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCAASRFTFSDFGMHWIRQAPGKCLEWVSYISSGSSTIYYADTVK | |
| | GRFTISRDNAKNSLYLQMNSLRAEDTAVYYCASSQSYYRGTLDYWGQGT | |
| | TVTVSS (SEQ ID NO: 233) | |
| 10F10 humanized | X₁VQLX₂ESGGGLVX₃PGGSLRLSC | DIQLTQSPSSLSASVGDRVTITCR |
| consensus | AASRFTFSDFGMHWVRQAPGKGLE | ASQSVTTSNYNYMHWFQQKPGKAP |
| sequence | WX₄X₅YISSGSSTIYYADX₆VKGRF | KLLIKFASNLESGVPSRFSGSGSG |
| | TISRDNAKNX₇LYLQMNSLRAEDT | TDFTLTISSLQPEDFATYYCQHSW |
| | AVYYCASSQSYYRGTLDYWGQGTT | EIPWTFGGGTKVEIK |
| | VTVSS (SEQ ID NO: 263) | (SEQ ID NO: 145) |
| | CDR1-RFTFSDF | CDR1-RASQSVTTSNYNYMH |
| | (SEQ ID NO: 138) | (SEQ ID NO: 141) |
| | CDR2-SSGSST | CDR2-FASNLES |
| | (SEQ ID NO: 139) | (SEQ ID NO: 142) |
| | CDR3-SQSYYRGTLDY | CDR3-QHSWEIPWT |
| | (SEQ ID NO: 140) | (SEQ ID NO: 143) |
| | Where X₁ is E or Q; X₂ | |
| | is V or L; X₃ is K or Q; | |
| | X₄ is V or I; X₅ is S or | |
| | A; X₆ is S or T; and X₇ | |
| | is S or T | |
| 10F10 humanized | X₁VQLX₂ESGGGLVX₃PGGSLRLSCAASRFTFSDFGMHWVRQAPGKGLEW | |
| consensus scFv | X₄X₅YISSGSSTIYYADX₆VKGRFTISRDNAKNX₇LYLQMNSLRAEDTAVY | |
| (VH-VL) | YCASSQSYYRGTLDYWGQGTTVTVSSGGGGSGGGGSGGGGSGGGGSDIQ | |
| | LTQSPSSLSASVGDRVTITCRASQSVTTSNYNYMHWFQQKPGKAPKLLI | |
| | KFASNLESGVPSRFSGSGSGTDFTLTISSLQPEDFATYYCQHSWEIPWT | |
| | FGGGTKVEIK (SEQ ID NO: 264) | |
| | Where X₁ is E or Q; X₂ is V or L; X₃ is K or Q; X₄ | |
| | is V or I; X₅ is S or A; X₆ is S or T; and X₇ is S | |
| | or T | |
| 10F10 humanized | DIQLTQSPSSLSASVGDRVTITCRASQSVTTSNYNYMHWFQQKPGKAPK | |
| consensus scFv | LLIKFASNLESGVPSRFSGSGSGTDFTLTISSLQPEDFATYYCQHSWEI | |
| (VL-VH) | PWTFGGGTKVEIKGGGGSGGGGSGGGGSGGGGSX₁VQLX₂ESGGGLVX₃P | |
| | GGSLRLSCAASRFTFSDFGMHWVRQAPGKGLEWX₄X₅YISSGSSTIYYAD | |
| | X₆VKGRFTISRDNAKNX₇LYLQMNSLRAEDTAVYYCASSQSYYRGTLDYW | |
| | GQGTTVTVSS (SEQ ID NO: 265) | |
| | Where X₁ is E or Q; X₂ is V or L; X₃ is K or Q; X₄ | |
| | is V or 1; X₅ is S or A; X₆ is S or T; and X₇ is S | |
| | or T | |
| 10F10 humanized | X₁VQLX₂ESGGGLVX₃PGGSLRLSCAASRFTFSDFGMHWVRQAPGKCLEW | |
| consensus scFv | X₄X₅YISSGSSTIYYADX₆VKGRFTISRDNAKNX₇LYLQMNSLRAEDTAVY | |
| (VH-VL) (with | YCASSQSYYRGTLDYWGQGTTVTVSSGGGGSGGGGSGGGGSGGGGSDIQ | |
| cysteine mutations | LTQSPSSLSASVGDRVTITCRASQSVTTSNYNYMHWFQQKPGKAPKLLI | |
| for disulfide bond | KFASNLESGVPSRFSGSGSGTDFTLTISSLQPEDFATYYCQHSWEIPWT | |
| formation) | FGCGTKVEIK (SEQ ID NO: 296) | |
| | Where X₁ is E or Q; X₂ is V or L; X₃ is K or Q; X₄ | |
| | is V or I; X₅ is S or A; X₆ is S or T; and X₇ is S | |
| | or T | |
| 10F10 humanized | DIQLTQSPSSLSASVGDRVTITCRASQSVTTSNYNYMHWFQQKPGKAPK | |
| consensus scFv | LLIKFASNLESGVPSRFSGSGSGTDFTLTISSLQPEDFATYYCQHSWEI | |
| (VL-VH) (with | PWTFGCGTKVEIKGGGGSGGGGSGGGGSGGGGSX₁VQLX₂ESGGGLVX₃P | |
| cysteine mutations | GGSLRLSCAASRFTFSDFGMHWVRQAPGKCLEWX₄X₅YISSGSSTIYYAD | |
| for disulfide bond | X₆VKGRFTISRDNAKNX₇LYLQMNSLRAEDTAVYYCASSQSYYRGTLDYW | |
| formation) | GQGTTVTVSS (SEQ ID NO: 297) | |
| | Where X₁ is E or Q; X₂ is V or L; X₃ is K or Q; X₄ | |
| | is V or I; X₅ is S or A; X₆ is S or T; and X₇ is S | |
| | or T | |
| 11F09 48*01 | EVQLVESGGGLVQPGGSLRLSCAA | DIQLTQSPSSLSASVGDRVTITCR |
| | SGFTFSDYGMHWVRQAPGKGLEWV | ASQGVSTSTYTYMHWFQQKPGKAP |
| | SYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 176) | (SEQ ID NO: 177) |
| | CDR1-GFTFSDY | CDR1-RASQGVSTSTYTYMH |
| | (SEQ ID NO: 163) | (SEQ ID NO: 178) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| 11F09 48*01 scFv | DIQLTQSPSSLSASVGDRVTITCRASQGVSTSTYTYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVQPGG | |
| | SLRLSCAASGFTFSDYGMHWVRQAPGKCLEWVSYISSGSSTFYYADTVK | |
| | GRFTISRDNAKNSLYLQMNSLRAEDTAVYYCGSSQSYYRGTMDYWGQGT | |
| | TVTVSS (SEQ ID NO: 179) | |
| 11F09 48*01 BM2 | EVQLVESGGGLVQPGGSLRLSCAA | DIQLTQSPSSLSASVGDRVTITCR |
| | SGFTFSDYGMHWVRQAPGKGLEWL | ASQGVSTSTYTYMHWFQQKPGKAP |
| | AYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 245) | (SEQ ID NO: 177) |
| | CDR1-GFTFSDY | CDR1-RASQGVSTSTYTYMH |
| | (SEQ ID NO: 163) | (SEQ ID NO: 178) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| 11F09 48*01 BM2 | DIQLTQSPSSLSASVGDRVTITCRASQGVSTSTYTYMHWFQQKPGKAPK | |
| scFv (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVQPGG | |
| | SLRLSCAASGFTFSDYGMHWVRQAPGKCLEWLAY1SSGSSTFYYADTVK | |
| | GRFTISRDNAKNSLYLQMNSLRAEDTAVYYCGSSQSYYRGTMDYWGQGT | |
| | TVTVSS (SEQ ID NO: 246) | |
| 11F09 21*05 | EVQLVESGGGLVKPGGSLRLSCEA | DIQLTQSPSSLSASVGDRVTITCR |
| | SGFTFSDYGMHWVRQAPGKGLEWL | ASQGVSTSTYTYMHWFQQKPGKAP |
| | AYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 161) | (SEQ ID NO: 177) |
| | CDR1-GFTFSDY | CDR1-RASQGVSTSTYTYMH |
| | (SEQ ID NO: 163) | (SEQ ID NO: 178) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| 11F09 21*05 scFv | DIQLTQSPSSLSASVGDRVTITCRASQGVSTSTYTYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCEASGFTFSDYGMHWVRQAPGKCLEWLAYISSGSSTFYYADTVK | |
| | GRFTISRDNAKNSLYLQMNSLRAEDTAVYYCGSSQSYYRGTMDYWGQGT | |
| | TVTVSS (SEQ ID NO: 202) | |
| 11F09 21*05 BM1 | EVQLVESGGGLVKPGGSLRLSCEA | DIQLTQSPSSLSASVGDRVTITCR |
| | SGFTFSDYGMHWVRQAPGKGLEWV | ASQGVSTSTYTYMHWFQQKPGKAP |
| | SYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 242) | (SEQ ID NO: 177) |
| | CDR1-GFTFSDY | CDR1-RASQGVSTSTYTYMH |
| | (SEQ ID NO: 163) | (SEQ ID NO: 178) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| 11F09 21*05 scFv | DIQLTQSPSSLSASVGDRVTITCRASQGVSTSTYTYMHWFQQKPGKAPK | |
| (VL-VH) BM1 | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCEASGFTFSDYGMHWVRQAPGKCLEWVSYISSGSSTFYYADTVK | |
| | GRFTISRDNAKNSLYLQMNSLRAEDTAVYYCGSSQSYYRGTMDYWGQGT | |
| | TVTVSS (SEQ ID NO: 243) | |
| 11F09 11*01 | QVQLVESGGGLVKPGGSLRLSCAA | DIQLTQSPSSLSASVGDRVTITCR |
| | SGFTFSDYGMHWVRQAPGKGLEWL | ASQGVSTSTYTYMHWFQQKPGKAP |
| | AYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 180) | (SEQ ID NO: 177) |
| | CDR1-GFTFSDY | CDR1-RASQGVSTSTYTYMH |
| | (SEQ ID NO: 163) | (SEQ ID NO: 178) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| 11F09 11*01 scFv | DIQLTQSPSSLSASVGDRVTITCRASQGVSTSTYTYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSQVQLVESGGGLVKPGG | |
| | SLRLSCAASGFTFSDYGMHWVRQAPGKCLEWLAYISSGSSTFYYADTVK | |
| | GRFTISRDNAKNSLYLQMNSLRAEDTAVYYCGSSQSYYRGTMDYWGQGT | |
| | TVTVSS (SEQ ID NO: 181) | |
| 11F09 11*01 BM1 | EVQLVESGGGLVKPGGSLRLSCAA | DIQLTQSPSSLSASVGDRVTITCR |
| | SGFTFSDYGMHWIRQAPGKGLEWV | ASQGVSTSTYTYMHWFQQKPGKAP |
| | SYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 247) | (SEQ ID NO: 177) |
| | CDR1-GFTFSDY | CDR1-RASQGVSTSTYTYMH |
| | (SEQ ID NO: 163) | (SEQ ID NO: 178) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| 11F09 11*01 BM1 | DIQLTQSPSSLSASVGDRVTITCRASQGVSTSTYTYMHWFQQKPGKAPK | |
| scFv (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCAASGFTFSDYGMHWIRQAPGKCLEWVSYISSGSSTFYYADTVK | |
| | GRFTISRDNAKNSLYLQMNSLRAEDTAVYYCGSSQSYYRGTMDYWGQGT | |
| | TVTVSS (SEQ ID NO: 248) | |
| 11F09 23*03 | EVQLLESGGGLVQPGGSLRLSCAA | DIQLTQSPSSLSASVGDRVTITCR |
| | SGFTFSDYGMHWVRQAPGKGLEWV | ASQGVSTSTYTYMHWFQQKPGKAP |
| | SYISSGSSTFYYADTVKGRFTISR | KLLIKFASNLESGVPSRFSGSGSG |
| | DNSKNTLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 182) | (SEQ ID NO: 177) |
| | CDR1-GFTFSDY | CDR1-RASQGVSTSTYTYMH |
| | (SEQ ID NO: 163) | (SEQ ID NO: 178) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| 11F09 23*03 scFv | DIQLTQSPSSLSASVGDRVTITCRASQGVSTSTYTYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLLESGGGLVQPGG | |
| | SLRLSCAASGFTFSDYGMHWVRQAPGKCLEWVSYISSGSSTFYYADTVK | |
| | GRFTISRDNSKNTLYLQMNSLRAEDTAVYYCGSSQSYYRGTMDYWGQGT | |
| | TVTVSS (SEQ ID NO: 183) | |
| 11F09 23*03-BM2 | EVQLLESGGGLVQPGGSLRLSCAA | DIQLTQSPSSLSASVGDRVTITCR |
| | SGFTFSDYGMHWVRQAPGKGLEWL | ASQGVSTSTYTYMHWFQQKPGKAP |
| | AYISSGSSTFYYADTVKGRFTISR | KLLIKFASNLESGVPSRFSGSGSG |
| | DNAKNTLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 250) | (SEQ ID NO: 177) |
| | CDR1-GFTFSDY | CDR1-RASQGVSTSTYTYMH |
| | (SEQ ID NO: 163) | (SEQ ID NO: 178) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| 11F09 23*03-BM2 | DIQLTQSPSSLSASVGDRVTITCRASQGVSTSTYTYMHWFQQKPGKAPK | |
| scFv (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLLESGGGLVQPGG | |
| | SLRLSCAASGFTFSDYGMHWVRQAPGKCLEWLAYISSGSSTFYYADTVK | |
| | GRFTISRDNAKNTLYLQMNSLRAEDTAVYYCGSSQSYYRGTMDYWGQGT | |
| | TVTVSS (SEQ ID NO: 251) | |
| 08E06 | EVQLQESGAEMVRPGASVKLSCKA | DIVMTQSPASLAVSLGQRATISCR |
| | SGYTFTSYWMHWVKQRPGQGLEWI | ASQSVSTSTYSYMHWYQQKPGQSP |
| | GKIDSSDSKTQYNQKFKDKATLTV | KLLITYASNLESGVPARFSGSGSG |
| | DKSSITVYMELNSLTSEDSAVYYC | TDFTLNIHPVEEEDIATYYCQHSW |
| | ARGGYLWFAYWGQGTLVTVSA | EIPWTFGGGTKLEIK |
| | (SEQ ID NO: 184) | (SEQ ID NO: 185) |
| | CDR1-GYTFTSY | CDR1-RASQSVSTSTYSYMH |
| | (SEQ ID NO: 186) | (SEQ ID NO: 189) |
| | CDR2-DSSDSK | CDR2-YASNLES |
| | (SEQ ID NO: 187) | (SEQ ID NO: 190) |
| | CDR3-GGYLWFAY | CDR3-QHSWEIPWT |
| | (SEQ ID NO: 188) | (SEQ ID NO: 143) |
| 08E06-humanized | QVQLVQSGAEVKKPGASVKVSCKA | DIVMTQSPDSLAVSLGERATINCR |
| variant 1 | SGYTFTSYWMHWVRQAPGQGLEWM | ASQSVSTSTYSYMHWYQQKPGQPP |
| | GKIDSSDSKTQYNQKFKDRVTMTR | KLLIYYASNLESGVPDRFSGSGSG |
| | DTSTSTVYMELSSLRSEDTAVYYC | TDFTLTISSLQAEDVAVYYCQHSW |
| | ARGGYLWFAYWGQGTLVTVSS | EIPWTFGGGTKVEIK |
| | (SEQ ID NO: 191) | (SEQ ID NO: 192) |
| | CDR1-GYTFTSY | CDR1-RASQSVSTSTYSYMH |
| | (SEQ ID NO: 186) | (SEQ ID NO: 189) |
| | CDR2-DSSDSK | CDR2-YASNLES |
| | (SEQ ID NO: 187) | (SEQ ID NO: 190) |
| | CDR3-GGYLWFAY | CDR3-QHSWEIPWT |
| | (SEQ ID NO: 188) | (SEQ ID NO: 143) |
| scFv 08E06 (VH- | QVQLVQSGAEVKKPGASVKVSCKASGYTFTSYWMHWVRQAPGQGLEWMG | |
| VL) humanized | KIDSSDSKTQYNQKFKDRVTMTRDTSTSTVYMELSSLRSEDTAVYYCAR | |
| variant 1 | GGYLWFAYWGQGTLVTVSSGGGGSGGGGSGGGGSGGGGSDIVMTQSPDS | |
| | LAVSLGERATINCRASQSVSTSTYSYMHWYQQKPGQPPKLLIYYASNLE | |
| | SGVPDRFSGSGSGTDFTLTISSLQAEDVAVYYCQHSWEIPWTFGGGTKV | |
| | EIK (SEQ ID NO: 193) | |
| scFv08E06 (VL- | DIVMTQSPDSLAVSLGERATINCRASQSVSTSTYSYMHWYQQKPGQPPK | |
| VH) humanized | LLIYYASNLESGVPDRFSGSGSGTDFTLTISSLQAEDVAVYYCQHSWEI | |
| variant 1 | PWTFGGGTKVEIKGGGGSGGGGSGGGGSGGGGSQVQLVQSGAEVKKPGA | |
| | SVKVSCKASGYTFTSYWMHWVRQAPGQGLEWMGKIDSSDSKTQYNQKFK | |
| | DRVTMTRDTSTSTVYMELSSLRSEDTAVYYCARGGYLWFAYWGQGTLVT | |
| | VSS (SEQ ID NO: 194) | |
| scFv 08E06 (VH- | QVQLVQSGAEVKKPGASVKVSCKASGYTFTSYWMHWVRQAPGQCLEWMG | |
| VL) humanized | KIDSSDSKTQYNQKFKDRVTMTRDTSTSTVYMELSSLRSEDTAVYYCAR | |
| variant 1 | GGYLWFAYWGQGTLVTVSSGGGGSGGGGSGGGGSGGGGSDIVMTQSPDS | |
| (with cysteine | LAVSLGERATINCRASQSVSTSTYSYMHWYQQKPGQPPKLLIYYASNLE | |
| mutations for | SGVPDRFSGSGSGTDFTLTISSLQAEDVAVYYCQHSWEIPWTFGCGTKV | |
| disulfide bond | EIK (SEQ ID NO: 294) | |
| formation) | | |
| scFv08E06 (VL- | DIVMTQSPDSLAVSLGERATINCRASQSVSTSTYSYMHWYQQKPGQPPK | |
| VH) humanized | LLIYYASNLESGVPDRFSGSGSGTDFTLTISSLQAEDVAVYYCQHSWEI | |
| variant 1 | PWTFGCGTKVEIKGGGGSGGGGSGGGGSGGGGSQVQLVQSGAEVKKPGA | |
| (with cysteine | SVKVSCKASGYTFTSYWMHWVRQAPGQCLEWMGKIDSSDSKTQYNQKFK | |
| mutations for | DRVTMTRDTSTSTVYMELSSLRSEDTAVYYCARGGYLWFAYWGQGTLVT | |
| disulfide bond | VSS (SEQ ID NO: 295) | |
| formation) | | |
| 08E06-humanized | QVQLVQSGAEVKKPGASVKVSCKA | DIVMTQSPASLAVSLGERATINCR |
| variant 2 | SGYTFTSYWMHWVRQAPGQGLEWM | ASQSVSTSTYSYMHWYQQKPGQPP |
| | GKIDSSDSKTQYNQKFKDRVTMTV | KLLITYASNLESGVPDRFSGSGSG |
| | DTSSSTVYMELSSLRSEDTAVYYC | TDFTLTISSLQAEDVATYYCQHSW |
| | ARGGYLWFAYWGQGTLVTVS | EIPWTFGGGTKVEIK |
| | (SEQ ID NO: 195) | (SEQ ID NO: 196) |
| | CDR1-GYTFTSY | CDR1-RASQSVSTSTYSYMH |
| | (SEQ ID NO: 186) | (SEQ ID NO: 189) |
| | CDR2-DSSDSK | CDR2-YASNLES |
| | (SEQ ID NO: 187) | (SEQ ID NO: 190) |
| | CDR3-GGYLWFAY | CDR3-QHSWEIPWT |
| | (SEQ ID NO: 188) | (SEQ ID NO: 143) |
| AB1011 | EVQLVESGGGLVKPGGSLRLSCAA | DIQLTQSPSSLSASVGDRVTITCR |
| (11F09-VH_BM1- | SGFTFSDYGMHWVRQAPGKGLEWV | ASQGVSTSTYTYMHWFQQKPGKAP |
| VK_BM1) | SYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 266) | (SEQ ID NO: 177) |
| | CDR1-GETESDY | CDR1-RASQGVSTSTYTYMH |
| | (SEQ ID NO: 163) | (SEQ ID NO: 178) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| AB1011 scFv | DIQLTQSPSSLSASVGDRVTITCRASQGVSTSTYTYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCAASGFTFSDYGMHWVRQAPGKCLEWVSYISSGSSTFYYADTVK | |
| | GRFTISRDNAKNSLYLQMNSLRAEDTAVYYCGSSQSYYRGTMDYWGQGT | |
| | TVTVSS (SEQ ID NO: 271) | |
| AB1012 | EVQLVESGGGLVKPGGSLRLSCAA | DIQLTQSPSSLSASVGDRVTITCR |
| (11F09-VH_BM1- | SGFTFSDYGMHWVRQAPGKGLEWV | ASQGVSTSTYTYLHWFQQKPGKAP |
| VK_BM1_M33L) | SYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 266) | (SEQ ID NO: 162) |
| | CDR1-GETESDY | CDR1-RASQGVSTSTYTYLH |
| | (SEQ ID NO: 163) | (SEQ ID NO: 165) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| AB1012 scFv | DIQLTQSPSSLSASVGDRVTITCRASQGVSTSTYTYLHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCAASGFTFSDYGMHWVRQAPGKCLEWVSYISSGSSTFYYADTVK | |
| | GRFTISRDNAKNSLYLQMNSLRAEDTAVYYCGSSQSYYRGTMDYWGQGT | |
| | TVTVSS (SEQ ID NO: 272) | |
| AB1013 | EVQLVESGGGLVKPGGSLRLSCAA | DIQLTQSPSSLSASVGDRVTITCR |
| (11F09-VHBM1- | SGFTFSDYGMHWVRQAPGKGLEWV | ASQGVSTSTYTYVHWFQQKPGKAP |
| VK_BM1_M33V) | SYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 266) | (SEQ ID NO: 267) |
| | CDR1-GETESDY | CDR1-RASQGVSTSTYTYVH |
| | (SEQ ID NO: 163) | (SEQ ID NO: 268) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| AB1013 scFv | DIQLTQSPSSLSASVGDRVTITCRASQGVSTSTYTYVHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCAASGFTFSDYGMHWVRQAPGKCLEWVSYISSGSSTFYYADTVK | |
| | GRFTISRDNAKNSLYLQMNSLRAEDTAVYYCGSSQSYYRGTMDYWGQGT | |
| | TVTVSS (SEQ ID NO: 273) | |
| AB1014 | EVQLVESGGGLVKPGGSLRLSCAA | DIQLTQSPSSLSASVGDRVTITCR |
| (11F09- | SGFTFSDYGMHWVRQAPGKGLEWV | ASQGVSTSTYTYMHWFQQKPGKAP |
| VH_BM1_M100cI- | SYISSGSSTFYYADTVKGRFTISR | KLLIKFASNLESGVPSRFSGSGSG |
| VK_BM1) | DNAKNSLYLQMNSLRAEDTAVYYC | TDFTLTISSLQPEDFATYYCQHSW |
| | GSSQSYYRGTIDYWGQGTTVTVSS | EIPWTFGGGTKVEIK |
| | (SEQ ID NO: 269) | (SEQ ID NO: 177) |
| | CDR1-GFTFSDY | CDR1-RASQGVSTSTYTYMH |
| | (SEQ ID NO: 163) | (SEQ ID NO: 178) |
| | CDR2-SSGSST | CDR2-FASNLES |
| | (SEQ ID NO: 139) | (SEQ ID NO: 142) |
| | CDR3-SQSYYRGTIDY | CDR3-QHSWEIPWT |
| | (SEQ ID NO: 270) | (SEQ ID NO: 143) |
| AB1014 scFv | DIQLTQSPSSLSASVGDRVTITCRASQGVSTSTYTYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCAASGFTFSDYGMHWVRQAPGKCLEWVSYISSGSSTFYYADTVK | |
| | GRFTISRDNAKNSLYLQMNSLRAEDTAVYYCGSSQSYYRGTIDYWGQGT | |
| | TVTVSS (SEQ ID NO: 274) | |
| AB1015 | EVQLVESGGGLVKPGGSLRLSCAA | DIQLTQSPSSLSASVGDRVTITCR |
| (11F09- | SGFTFSDYGMHWVRQAPGKGLEWV | ASQGVSTSTYTYLHWFQQKPGKAP |
| VH_BM1_M100cI- | SYISSGSSTFYYADTVKGRFTISR | KLLIKFASNLESGVPSRFSGSGSG |
| VK_BM1_M33L) | DNAKNSLYLQMNSLRAEDTAVYYC | TDFTLTISSLQPEDFATYYCQHSW |
| | GSSQSYYRGTIDYWGQGTTVTVSS | EIPWTFGGGTKVEIK |
| | (SEQ ID NO: 269) | (SEQ ID NO: 162) |
| | CDR1-GFTFSDY | CDR1-RASQGVSTSTYTYLH |
| | (SEQ ID NO: 163 | (SEQ ID NO: 165) |
| | CDR2-SSGSST | CDR2-FASNLES |
| | (SEQ ID NO: 139) | (SEQ ID NO: 142) |
| | CDR3-SQSYYRGTIDY | CDR3-QHSWEIPWT |
| | (SEQ ID NO: 270) | (SEQ ID NO: 143) |
| AB1015 scFv | DIQLTQSPSSLSASVGDRVTITCRASQGVSTSTYTYLHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCAASGFTFSDYGMHWVRQAPGKCLEWVSYISSGSSTFYYADTVK | |
| | GRFTISRDNAKNSLYLQMNSLRAEDTAVYYCGSSQSYYRGTIDYWGQGT | |
| | TVTVSS (SEQ ID NO: 275) | |
| AB1016 | EVQLVESGGGLVKPGGSLRLSCAA | DIQLTQSPSSLSASVGDRVTITCR |
| (11F09- | SGFTFSDYGMHWVRQAPGKGLEWV | ASQGVSTSTYTYVHWFQQKPGKAP |
| VH_BM1_M100cI- | SYISSGSSTFYYADTVKGRFTISR | KLLIKFASNLESGVPSRFSGSGSG |
| VK_BM1_M33V) | DNAKNSLYLQMNSLRAEDTAVYYC | TDFTLTISSLQPEDFATYYCQHSW |
| | GSSQSYYRGTIDYWGQGTTVTVSS | EIPWTFGGGTKVEIK |
| | (SEQ ID NO: 269) | (SEQ ID NO: 267) |
| | CDR1-GFTFSDY | CDR1-RASQGVSTSTYTYVH |
| | (SEQ ID NO: 163) | (SEQ ID NO: 268) |
| | CDR2-SSGSST | CDR2-FASNLES |
| | (SEQ ID NO: 139) | (SEQ ID NO: 142) |
| | CDR3-SQSYYRGTIDY | CDR3-QHSWEIPWT |
| | (SEQ ID NO: 270) | (SEQ ID NO: 143) |
| AB1016 scFv | DIQLTQSPSSLSASVGDRVTITCRASQGVSTSTYTYVHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCAASGFTFSDYGMHWVRQAPGKCLEWVSYISSGSSTFYYADTVK | |
| | GRFTISRDNAKNSLYLQMNSLRAEDTAVYYCGSSQSYYRGTIDYWGQGT | |
| | TVTVSS (SEQ ID NO: 276) | |
| AB1017 | EVQLVESGGGLVKPGGSLRLSCEA | DIQLTQSPSSLSASVGDRVTITCR |
| (11F09-VH_BM2- | SGFTFSDYGMHWVRQAPGKGLEWL | ASQGVSTSTYTYMHWFQQKPGKAP |
| VKBM1) | AYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 161) | (SEQ ID NO: 177) |
| | CDR1-GFTFSDY | CDR1-RASQGVSTSTYTYMH |
| | (SEQ ID NO: 163) | (SEQ ID NO: 178) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| AB1017 scFv | DIQLTQSPSSLSASVGDRVTITCRASQGVSTSTYTYMHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCEASGFTFSDYGMHWVRQAPGKCLEWLAYISSGSSTFYYADTVK | |
| | GRFTISRDNAKNSLYLQMNSLRAEDTAVYYCGSSQSYYRGTMDYWGQGT | |
| | TVTVSS (SEQ ID NO: 277) | |
| AB1018 | EVQLVESGGGLVKPGGSLRLSCEA | DIQLTQSPSSLSASVGDRVTITCR |
| (11F09-VH_BM2- | SGFTFSDYGMHWVRQAPGKGLEWL | ASQGVSTSTYTYLHWFQQKPGKAP |
| VKBM1M33L) | AYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 161) | (SEQ ID NO: 162) |
| | CDR1-GETFSDY | CDR1-RASQGVSTSTYTYLH |
| | (SEQ ID NO: 163) | (SEQ ID NO: 165) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |
| AB1018 scFv | DIQLTQSPSSLSASVGDRVTITCRASQGVSTSTYTYLHWFQQKPGKAPK | |
| (VL-VH) | LLIKFASNLESGVPSRFSGSGSGTDFTLTISSLQPEDFATYYCQHSWEI | |
| | PWTFGCGTKVEIKGGGGSGGGGSGGGGSGGGGSEVQLVESGGGLVKPGG | |
| | SLRLSCEASGFTFSDYGMHWVRQAPGKCLEWLAYISSGSSTFYYADTVK | |
| | GRFTISRDNAKNSLYLQMNSLRAEDTAVYYCGSSQSYYRGTMDYWGQGT | |
| | TVTVSS (SEQ ID NO: 278) | |
| AB1019 | EVQLVESGGGLVKPGGSLRLSCEA | DIQLTQSPSSLSASVGDRVTITCR |
| (11F09-VH_BM2- | SGFTFSDYGMHWVRQAPGKGLEWL | ASQGVSTSTYTYVHWFQQKPGKAP |
| VKBM1M33V) | AYISSGSSTFYYADTVKGRFTISR | KLLIKFASNLESGVPSRFSGSGSG |
| | DNAKNSLYLQMNSLRAEDTAVYYC | TDFTLTISSLQPEDFATYYCQHSW |
| | GSSQSYYRGTMDYWGQGTTVTVSS | EIPWTFGGGTKVEIK |
| | (SEQ ID NO: 161) | (SEQ ID NO: 267) |
| | CDR1-GETFSDY | CDR1-RASQGVSTSTYTYVH |
| | (SEQ ID NO: 163) | (SEQ ID NO: 268) |
| | CDR2-SSGSST | CDR2-FASNLES |
| | (SEQ ID NO: 139) | (SEQ ID NO: 142) |
| | CDR3-SQSYYRGTMDY | CDR3-QHSWEIPWT |
| | (SEQ ID NO: 164) | (SEQ ID NO: 143) |

| | |
|---|---|
| AB1019 scFv | DIQLTQSPSSLSASVGDRVTITCRASQGVSTSTYTYVHWFQQKPGKAPK |
| (VL-VH) | LLIKFASNLESGVPSRFSGSGSGTDFTLTISSLQPEDFATYYCQHSWEI |
| | PWTFGCGTKVEIKGGGGSGGGGSGGGGSGGGGSEVQLVESGGGLVKPGG |
| | SLRLSCEASGFTFSDYGMHWVRQAPGKCLEWLAYISSGSSTFYYADTVK |
| | GRFTISRDNAKNSLYLQMNSLRAEDTAVYYCGSSQSYYRGTMDYWGQGT |
| | TVTVSS (SEQ ID NO: 279) |

TABLE 2A
CDRs for illustrative anti-5T4 binding antigen binding domains (Kabat)

| Ab | VH- | | | | VL- | |
|---|---|---|---|---|---|---|
| Name | CDR1 | VH-CDR2 | VH-CDR3 | VL-CDR1 | CDR2 | VL-CDR3 |
| 140 | DFGMH | YISSGSSTIYYADTVKG | SQSYYRGTLDY | RASQSVTTSNYNYMH | FASNLES | QHSWEIPWT |
| | SEQ ID | SEQ ID NO: 473 | SEQ ID NO: 140 | SEQ ID NO: 141 | SEQ ID | SEQ ID |
| | NO: 472 | | | | NO: 142 | NO: 143 |
| 141 | DFGMH | YISSGSSTIYYADSVKG | SQSYYRGTLDY | RASQSVTTSNYNYMH | FASNLES | QHSWEIPWT |
| | SEQ ID | SEQ ID NO: 474 | SEQ ID NO: 140 | SEQ ID NO: 141 | SEQ ID | SEQ ID |
| | NO: 472 | | | | NO: 142 | NO: 143 |
| 142 | DFGMH | YISSGSSTIYYADSVKG | SQSYYRGTLDY | RASQSVTTSNYNYLH | FASNLES | QHSWEIPWT |
| | SEQ ID | SEQ ID NO: 474 | SEQ ID NO: 140 | SEQ ID NO: 290 | SEQ ID | SEQ ID |
| | NO: 472 | | | | NO: 142 | NO: 143 |
| 143 | DFGMH | YISSGSSTIYYADSVKG | SQSYYRGTLDY | RASQSVTTSNYNYVH | FASNLES | QHSWEIPWT |
| | SEQ ID | SEQ ID NO: 474 | SEQ ID NO: 140 | SEQ ID NO: 292 | SEQ ID | SEQ ID |
| | NO: 472 | | | | NO: 142 | NO: 143 |
| 144 | DFGMH | YISSGSSTIYYADXVKG | SQSYYRGTLDY | RASQSVTTSNYNYX₈H | FASNLES | QHSWEIPWT |
| | SEQ ID | X₆ = S or T | SEQ ID NO: 140 | X₈ = L, M or V | SEQ ID | SEQ ID |
| | NO: 472 | SEQ ID NO: 475 | | SEQ ID NO: 541 | NO: 142 | NO: 143 |
| 145 | DYYMD | YIFPNDASTTYNEKFKG | SRDADY | SAKSSVSYIH | DTSYLGS | QQWSSYPYT |
| | SEQ ID | SEQ ID NO: 477 | SEQ ID NO: 153 | SEQ ID NO: 154 | SEQ ID | SEQ ID |
| | NO: 476 | | | | NO: 155 | NO: 156 |
| 146 | DYGMH | YISSGSSTFYYADTVKG | SQSYYRGTMDY | RASQGVSTSTYTYLH | FASNLES | QHSWEIPWT |
| | SEQ ID | SEQ ID NO: 479 | SEQ ID NO: 164 | SEQ ID NO: 165 | SEQ ID | SEQ ID |
| | NO: 478 | | | | NO: 142 | NO: 143 |
| 147 | DYGMH | YISSGSSTFYYADTVKG | SQSYYRGTMDY | RASQGVSTSTYTYMH | FASNLES | QHSWEIPWT |
| | SEQ ID | SEQ ID NO: 479 | SEQ ID NO: 164 | SEQ ID NO: 178 | SEQ ID | SEQ ID |
| | NO: 478 | | | | NO: 142 | NO: 143 |
| 148 | DYGMH | YISSGSSTFYYADTVKG | SQSYYRGTMDY | RASQGVSTSTYTYVH | FASNLES | QHSWEIPWT |
| | SEQ ID | SEQ ID NO: 479 | SEQ ID NO: 164 | SEQ ID NO: 268 | SEQ ID | SEQ ID |
| | NO: 478 | | | | NO: 142 | NO: 143 |
| 149 | DYGMH | YISSGSSTFYYADTVKG | SQSYYRGTIDY | RASQGVSTSTYTYMH | FASNLES | QHSWEIPWT |
| | SEQ ID | SEQ ID NO: 479 | SEQ ID NO: 270 | SEQ ID NO: 178 | SEQ ID | SEQ ID |
| | NO: 478 | | | | NO: 142 | NO: 143 |
| 150 | DYGMH | YISSGSSTFYYADTVKG | SQSYYRGTIDY | RASQGVSTSTYTYLH | FASNLES | QHSWEIPWT |
| | SEQ ID | SEQ ID NO: 479 | SEQ ID NO: 270 | SEQ ID NO: 165 | SEQ ID | SEQ ID |
| | NO: 478 | | | | NO: 142 | NO: 143 |
| 151 | DYGMH | YISSGSSTFYYADTVKG | SQSYYRGTIDY | RASQGVSTSTYTYVH | FASNLES | QHSWEIPWT |
| | SEQ ID | SEQ ID NO: 479 | SEQ ID NO: 270 | SEQ ID NO: 268 | SEQ ID | SEQ ID |
| | NO: 478 | | | | NO: 142 | NO: 143 |
| 152 | SYWMH | KIDSSDSKTQYNQKFKD | GGYLWFAY | RASQSVSTSTYSYMH | YASNLES | QHSWEIPWT |
| | SEQ ID | SEQ ID NO: 481 | SEQ ID NO: 188 | SEQ ID NO: 189 | SEQ ID | SEQ ID |
| | NO: 480 | | | | NO: 190 | NO: 143 |

TABLE 2B
CDRs for illustrative anti-5T4 binding antigen binding domains (Chothia)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 153 | RFTFSDF | SGSS | QSYYRGTLD | SQSVTTSNYNY | FAS | SWEIPW |
| | SEQ ID | SEQ ID | SEQ ID NO: 483 | SEQ ID NO: 484 | SEQ ID | SEQ ID |
| | NO: 138 | NO: 482 | | | NO: 485 | NO: 486 |
| 154 | GYKFTDY | PNDA | RDAD | KSSVSY | DTS | WSSYPY |
| | SEQ ID | SEQ ID | SEQ ID NO: 488 | SEQ ID NO: 489 | SEQ ID | SEQ ID |
| | NO: 152 | NO: 487 | | | NO: 490 | NO: 491 |
| 155 | GETESDY | SGSS | QSYYRGTMD | SQGVSTSTYTY | FAS | SWEIPW |
| | SEQ ID | SEQ ID | SEQ ID NO: 492 | SEQ ID NO: 493 | SEQ ID | SEQ ID |
| | NO: 163 | NO: 482 | | | NO: 485 | NO: 486 |
| 156 | GETESDY | SGSS | QSYYRGTID | SQGVSTSTYTY | FAS | SWEIPW |
| | SEQ ID | SEQ ID | SEQ ID NO: 494 | SEQ ID NO: 493 | SEQ ID | SEQ ID |
| | NO: 163 | NO: 482 | | | NO: 485 | NO: 486 |
| 157 | GYTFTSY | SSDS | GYLWFA | SQSVSTSTYSY | YAS | SWEIPW |
| | SEQ ID | SEQ ID | SEQ ID NO: 496 | SEQ ID NO: 497 | SEQ ID | SEQ ID |
| | NO: 186 | NO: 495 | | | NO: 498 | NO: 486 |

TABLE 2C
CDRs for illustrative anti-5T4 binding antigen binding domains (IMGT)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 158 | RFTFSDFG | ISSGSSTI | ASSQSYYRGTLDY | QSVTTSNYNY | FAS | QHSWEIPWT |
| | SEQ ID | SEQ ID NO: 500 | SEQ ID NO: 501 | SEQ ID NO: 502 | SEQ ID | SEQ ID |
| | NO: 499 | | | | NO: 485 | NO: 143 |
| 159 | GYKFTDYY | IFPNDAST | ARSRDADY | SSVSY | DTS | QQWSSYPYT |
| | SEQ ID | SEQ ID NO: 504 | SEQ ID NO: 505 | SEQ ID NO: 506 | SEQ ID | SEQ ID |
| | NO: 503 | | | | NO: 490 | NO: 156 |
| 160 | GETESDYG | ISSGSSTF | GSSQSYYRGTMDY | QGVSTSTYTY | FAS | QHSWEIPWT |
| | SEQ ID | SEQ ID NO: 508 | SEQ ID NO: 509 | SEQ ID NO: 510 | SEQ ID | SEQ ID |
| | NO: 507 | | | | NO: 485 | NO: 143 |
| 161 | GETESDYG | ISSGSSTF | GSSQSYYRGTIDY | QGVSTSTYTY | FAS | QHSWEIPWT |
| | SEQ ID | SEQ ID NO: 508 | SEQ ID NO: 511 | SEQ ID NO: 510 | SEQ ID | SEQ ID |
| | NO: 507 | | | | NO: 485 | NO: 143 |
| 162 | GYTFTSYW | IDSSDSKT | ARGGYLWFAY | QSVSTSTYSY | YAS | QHSWEIPWT |
| | SEQ ID | SEQ ID NO: 513 | SEQ ID NO: 514 | SEQ ID NO: 515 | SEQ ID | SEQ ID |
| | NO: 512 | | | | NO: 498 | NO: 143 |

TABLE 2D
CDRs for illustrative anti-5T4 binding antigen binding domains (Honegger)

| Ab | | | | | | VL- |
|---|---|---|---|---|---|---|
| Name | VH-CDR1 | VH-CDR2 | VH-CDR3 | VL-CDR1 | VL-CDR2 | CDR3 |
| 163 | ASRFTFSDFG | ISSGSSTIYYADTVKGR | SQSYYRGTLD | ASQSVTTSNYNY | FASNLESGVPAR | SWEIPW |
| | SEQ ID | SEQ ID NO: 517 | SEQ ID NO: 518 | SEQ ID NO: 519 | SEQ ID NO: 520 | SEQ ID |
| | NO: 516 | | | | | NO: 486 |
| 164 | ASRFTFSDFG | ISSGSSTIYYADSVKGR | SQSYYRGTLD | ASQSVTTSNYNY | FASNLESGVPSR | SWEIPW |
| | SEQ ID | SEQ ID NO: 521 | SEQ ID NO: 518 | SEQ ID NO: 519 | SEQ ID NO: 522 | SEQ ID |
| | NO: 516 | | | | | NO: 486 |
| 165 | ASRFTFSDFG | ISSGSSTIYYADX₆VKGR | SQSYYRGTLD | ASQSVTTSNYNY | FASNLESGVPSR | SWEIPW |
| | SEQ ID | X₆ = S or T | SEQ ID NO: 518 | SEQ ID NO: 519 | SEQ ID NO: 522 | SEQ ID |
| | NO: 516 | SEQ ID NO: 523 | | | | NO: 486 |
| 166 | ASRFTFSDFG | ISSGSSTIYYADX₆VKGR | SQSYYRGTLD | ASQSVTTSNYNY | FASNLESGVPX₉R | SWEIPW |
| | SEQ ID | X₆ = S or T | SEQ ID NO: 518 | SEQ ID NO: 519 | X₉ = A or S | SEQ ID |
| | NO: 516 | SEQ ID NO: 523 | | | SEQ ID NO: 542 | NO: 486 |
| 167 | ASGYKFTDYY | IFPNDASTTYNEKFKGK | SRDAD | AKSSVSY | DTSYLGSGIPAR | WSSYPY |
| | SEQ ID | SEQ ID NO: 525 | SEQ ID NO: 526 | SEQ ID NO: 527 | SEQ ID NO: 528 | SEQ ID |
| | NO: 524 | | | | | NO: 491 |
| 168 | ASGFTFSDYG | ISSGSSTFYYADTVKGR | SQSYYRGTMD | ASQGVSTSTYTY | FASNLESGVPSR | SWEIPW |
| | SEQ ID | SEQ ID NO: 530 | SEQ ID NO: 531 | SEQ ID NO: 532 | SEQ ID NO: 522 | SEQ ID |
| | NO: 529 | | | | | NO: 486 |
| 169 | ASGFTFSDYG | ISSGSSTFYYADTVKGR | SQSYYRGTID | ASQGVSTSTYTY | FASNLESGVPSR | SWEIPW |
| | SEQ ID | SEQ ID NO: 530 | SEQ ID NO: 533 | SEQ ID NO: 532 | SEQ ID NO: 522 | SEQ ID |
| | NO: 529 | | | | | NO: 486 |
| 170 | ASGYTFTSYW | IDSSDSKTQYNQKFKDK | GGYLWFA | ASQSVSTSTYSY | YASNLESGVPAR | SWEIPW |
| | SEQ ID | SEQ ID NO: 535 | SEQ ID NO: 536 | SEQ ID NO: 537 | SEQ ID NO: 538 | SEQ ID |
| | NO: 534 | | | | | NO: 486 |
| 171 | ASGYTFTSYW | IDSSDSKTQYNQKFKDR | GGYLWFA | ASQSVSTSTYSY | YASNLESGVPDR | SWEIPW |
| | SEQ ID | SEQ ID NO: 539 | SEQ ID NO: 536 | SEQ ID NO: 537 | SEQ ID NO: 540 | SEQ ID |
| | NO: 534 | | | | | NO: 486 |

In various embodiments, the second antigen binding site or second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Kabat numbering scheme), respectively:

SEQ ID NOs: 472, 473, 140, 141, 142 and 143;

SEQ ID NOs: 472, 474, 140, 141, 142 and 143;

SEQ ID NOs: 472, 474, 140, 290, 142 and 143;

SEQ ID NOs: 472, 474, 140, 292, 142 and 143;

SEQ ID NOs: 472, 475, 140, 141, 142 and 143;

SEQ ID NOs: 472, 475, 140, 541, 142 and 143;

SEQ ID NOs: 476, 477, 153, 154, 155 and 156;

SEQ ID NOs: 478, 479, 164, 165, 142 and 143;

SEQ ID NOs: 478, 479, 164, 178, 142 and 143;

SEQ ID NOs: 478, 479, 164, 268, 142 and 143;

SEQ ID NOs: 478, 479, 270, 165, 142 and 143;

SEQ ID NOs: 478, 479, 270, 178, 142 and 143;

SEQ ID NOs: 478, 479, 270, 268, 142 and 143; or

SEQ ID NOs: 480, 481, 188, 189, 190 and 143.

In various embodiments, the second antigen binding site or second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Kabat numbering scheme), respectively:

SEQ ID NOs: 472, 473, 140, 141, 142 and 143;

SEQ ID NOs: 472, 474, 140, 141, 142 and 143;

SEQ ID NOs: 472, 474, 140, 290, 142 and 143;

SEQ ID NOs: 472, 474, 140, 292, 142 and 143;

SEQ ID NOs: 472, 475, 140, 141, 142 and 143; or

SEQ ID NOs: 472, 475, 140, 541, 142 and 143.

In some embodiments, the second antigen binding site or second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 472, 473, 140, 141, 142 and 143. In some embodiments, the present antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 472, 474, 140, 141, 142 and 143. In some embodiments, the present antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 472, 474, 140, 290, 142 and 143. In some embodiments, the present antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 472, 474, 140, 292, 142 and 143. In some embodiments, the present antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 472, 475, 140, 141, 142 and 143. In some embodiments, the present antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 472, 475, 140, 541, 142 and 143.

In various embodiments, the second antigen binding site or second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Chothia numbering scheme), respectively:

SEQ ID NOs: 138, 482, 483, 484, 485 and 486;

SEQ ID NOs: 152, 487, 488, 489, 490 and 491;

SEQ ID NOs: 163, 482, 492, 493, 485 and 486;

SEQ ID NOs: 163, 482, 492, 494, 485 and 486; or

SEQ ID NOs: 186, 495, 496, 497, 498 and 486.

In various embodiments, the second antigen binding site or second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Chothia numbering scheme), respectively: SEQ ID NOs: 138, 482, 483, 484, 485 and 486.

In various embodiments, the second antigen binding site or second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to IMGT numbering scheme), respectively:

SEQ ID NOs: 499, 500, 501, 502, 485 and 143;

SEQ ID NOs: 503, 504, 505, 506, 490 and 156;

SEQ ID NOs: 507, 508, 509, 510, 485 and 143;

SEQ ID NOs: 507, 508, 511, 510, 485 and 143; or

SEQ ID NOs: 512, 513, 514, 515, 498 and 143.

In various embodiments, the second antigen binding site or second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to IMGT numbering scheme), respectively: SEQ ID NOs: 499, 500, 501, 502, 485 and 143.

In various embodiments, the second antigen binding site or second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Honegger numbering scheme), respectively:

SEQ ID NOs: 516, 517, 518, 519, 520 and 486;

SEQ ID NOs: 516, 521, 518, 519, 522 and 486;

SEQ ID NOs: 516, 523, 518, 519, 522 and 486;

SEQ ID NOs: 516, 523, 518, 519, 542 and 486;

SEQ ID NOs: 524, 525, 526, 527, 528 and 491;

SEQ ID NOs: 529, 530, 531, 532, 522 and 486;

SEQ ID NOs: 529, 530, 533, 532, 522 and 486;

SEQ ID NOs: 534, 535, 536, 537, 538 and 486; or

SEQ ID NOs: 534, 539, 536, 537, 540 and 486.

In various embodiments, the second antigen binding site or second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Honegger numbering scheme), respectively:

SEQ ID NOs: 516, 517, 518, 519, 520 and 486;

SEQ ID NOs: 516, 521, 518, 519, 522 and 486;

SEQ ID NOs: 516, 523, 518, 519, 522 and 486; or

SEQ ID NOs: 516, 523, 518, 519, 542 and 486.

In various embodiments, the second antigen binding site or second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Honegger numbering scheme), respectively: SEQ ID NOs: 516, 517, 518, 519, 520 and 486. In various embodiments, the present antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Honegger numbering scheme), respectively: SEQ ID NOs: 516, 517, 518, 519, 522 and 486. In various embodiments, the present antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Honegger numbering scheme), respectively: SEQ ID NOs: 516, 523, 518, 519, 522 and 486. In various embodiments, the present antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Honegger numbering scheme), respectively: SEQ ID NOs: 516, 523, 518, 519, 542 and 486.

In certain embodiments, the second antigen-binding site that binds 5T4 (e.g., human 5T4) comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 2, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in Table 2. In certain embodiments, the second antigen-binding site comprises the heavy chain CDR1, CDR2, and CDR3, and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), or any other CDR determination method known in the art, of the VH and VL sequences of an antigen-binding site disclosed in Table 2. In certain embodiments, the second antigen-binding site comprises the heavy chain CDR1, CDR2, and CDR3, and the light chain CDR1, CDR2, and CDR3 of an antibody disclosed in Tables 2, 2A, 2B, 2C or 2D.

In certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:263, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:145. In certain embodiments, the antigen-biding site comprises a VL with a G100C substitution relative to SEQ ID NO:145. In certain embodiments, the VH comprises CDR1, CDR2, and CDR3 comprising the amino acid sequences of SEQ ID NOs: 138, 139, and 140, respectively. In certain embodiments, the VL comprises CDR1, CDR2, and CDR3 comprising the amino acid sequences of SEQ ID NOs: 141, 142, and 143, respectively.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:264 or SEQ ID NO:265.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:296 or SEQ ID NO:297.

In certain embodiments, the second antigen-binding site comprises (a) a VH that comprises CDR1, CDR2, and CDR3 comprising the amino acid sequences of SEQ ID NOs: 138, 139, and 140, respectively; and (b) a VL that comprises CDR1, CDR2, and CDR3 comprising the amino acid sequences of SEQ ID NOs: 141, 142, and 143, respectively.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:144, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:145. In certain embodiments, the second antigen-binding site that binds 5T4 comprises a VH that comprises the amino acid sequence of SEQ ID NO:144, and a VL that comprises the amino acid sequence of SEQ ID NO:145. In certain embodiments, the antigen-biding site comprises a VH with a G44C substitution relative to SEQ ID NO:144. In certain embodiments, the antigen-biding site comprises a VL with a G100C substitution relative to SEQ ID NO:145. In certain embodiments, the VH comprises CDR1, CDR2, and CDR3 comprising the amino acid sequences of SEQ ID NOs: 138, 139, and 140, respectively. In certain embodiments, the VL comprises CDR1, CDR2, and CDR3 comprising the amino acid sequences of SEQ ID NOs: 141, 142, and 143, respectively. In certain embodiments, the VL comprises a substitution of leucine (L) at position 33, according to the Kabat numbering scheme. Accordingly, in some embodiments, the VL comprises the amino acid sequence of SEQ ID NO:289 or the VL comprises CDR1, CDR2, and CDR3 comprising the amino acid sequences of SEQ ID NOs: 290, 142, and 143. In certain embodiments, the VL comprises a substitution of valine (V) at position 33, according to the Kabat numbering scheme. Accordingly, in some embodiments, the VL comprises the amino acid sequence of SEQ ID NO:291 or the VL comprises CDR1, CDR2, and CDR3 comprising the amino acid sequences of SEQ ID NOs: 292, 142, and 143.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:166, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:145. In certain embodiments, the VH comprises CDR1, CDR2, and CDR3 comprising the amino acid sequences of SEQ ID NOs: 138, 139, and 140, respectively. In certain embodiments, the VL comprises CDR1, CDR2, and CDR3 comprising the amino acid sequences of SEQ ID NOs: 141, 142, and 143, respectively.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:168, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:145. In certain embodiments, the VH comprises CDR1, CDR2, and CDR3 comprising the amino acid sequences of SEQ ID NOs: 138, 139, and 140, respectively. In certain embodiments, the VL comprises CDR1, CDR2, and CDR3 comprising the amino acid sequences of SEQ ID NOs: 141, 142, and 143, respectively. In certain embodiments, the VH comprises a substitution of serine (S) at position 62, according to the Kabat numbering scheme.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:236, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:145. In certain embodiments, the VH comprises CDR1, CDR2, and CDR3 comprising the amino acid sequences of SEQ ID NOs: 138, 139, and 140, respectively. In certain embodiments, the VL comprises CDR1, CDR2, and CDR3 comprising the amino acid sequences of SEQ ID NOs: 141, 142, and 143, respectively. In certain embodiments, the VH comprises a substitution of serine (S) at position 62, according to the Kabat numbering scheme.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:170, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:145. In certain embodiments, the VH comprises CDR1, CDR2, and CDR3 comprising the amino acid sequences of SEQ ID NOs: 138, 139, and 140, respectively. In certain embodiments, the VL comprises CDR1, CDR2, and CDR3 comprising the amino acid sequences of SEQ ID NOs: 141, 142, and 143, respectively. In certain embodiments, the VH comprises a substitution of serine (S) at position 62, according to the Kabat numbering scheme.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:228, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:145. In certain embodiments, the VH comprises CDR1, CDR2, and CDR3 comprising the amino acid sequences of SEQ ID NOs: 138, 139, and 140, respectively. In certain embodiments, the VL comprises CDR1, CDR2, and CDR3 comprising the amino acid sequences of SEQ ID NOs: 141, 142, and 143, respectively. In certain embodiments, the VH comprises a substitution of serine (S) at position 62, according to the Kabat numbering scheme.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:172, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:145. In certain embodiments, the VH comprises CDR1, CDR2, and CDR3 comprising the amino acid sequences of SEQ ID NOs: 138, 139, and 140, respectively. In certain embodiments, the VL comprises CDR1, CDR2, and CDR3 comprising the amino acid sequences of SEQ ID NOs: 141, 142, and 143, respectively. In certain embodiments, the VH comprises a substitution of serine (S) at position 62, according to the Kabat numbering scheme.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:174, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:145. In certain embodiments, the VH comprises CDR1, CDR2, and CDR3 comprising the amino acid sequences of SEQ ID NOs: 138, 139, and 140, respectively. In certain embodiments, the VL comprises CDR1, CDR2, and CDR3 comprising the amino acid sequences of SEQ ID NOs: 141, 142, and 143, respectively. In certain embodiments, the VH comprises a substitution of serine (S) at position 62, according to the Kabat numbering scheme.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:232, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:145. In certain embodiments, the VH comprises CDR1, CDR2, and CDR3 comprising the amino acid sequences of SEQ ID NOs: 138, 139, and 140, respectively. In certain embodiments, the VL comprises CDR1, CDR2, and CDR3 comprising the amino acid sequences of SEQ ID NOs: 141, 142, and 143, respectively. In certain embodiments, the VH comprises a substitution of serine (S) at position 62, according to the Kabat numbering scheme.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:146, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:147. In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:146, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:147. In certain embodiments, the second antigen-binding site that binds 5T4 comprises a VH that comprises an amino acid sequence at least 95% identical to the amino acid sequence of SEQ ID NO:146, and a VL that comprises an amino acid sequence at least 95% identical to the amino acid sequence of SEQ ID NO:147. In certain embodiments, the second antigen-binding site that binds 5T4 comprises a VH that comprises an amino acid sequence at least 96% identical to the amino acid sequence of SEQ ID NO:146, and a VL that comprises an amino acid sequence at least 96% identical to the amino acid sequence of SEQ ID NO:147. In certain embodiments, the second antigen-binding site that binds 5T4 comprises a VH that comprises an amino acid sequence at least 97% identical to the amino acid sequence of SEQ ID NO:146, and a VL that comprises an amino acid sequence at least 97% identical to the amino acid sequence of SEQ ID NO:147. In certain embodiments, the second antigen-binding site that binds 5T4 comprises a VH that comprises an amino acid sequence at least 98% identical to the amino acid sequence of SEQ ID NO:146, and a VL that comprises an amino acid sequence at least 98% identical to the amino acid sequence of SEQ ID NO:147. In certain embodiments, the second antigen-binding site that binds 5T4 comprises a VH that comprises an amino acid sequence at least 99% identical to the amino acid sequence of SEQ ID NO:146, and a VL that comprises an amino acid sequence at least 99% identical to the amino acid sequence of SEQ ID NO:147. In certain embodiments, the second antigen-binding site that binds 5T4 comprises a VH that comprises the amino acid sequence of SEQ ID NO:146, and a VL that comprises the amino acid sequence of SEQ ID NO:147.

In certain embodiments, the second antigen-binding site is present as an scFv, wherein the scFv comprises a VH comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:146 and a VL comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:147.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:148 or SEQ ID NO:149.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:148. In certain embodiments, the second antigen-binding site is present as an scFv, wherein the scFv comprises an amino acid sequence at least 95%, identical to the amino acid sequence of SEQ ID NO:148. In certain embodiments, the second antigen-binding site is present as an scFv, wherein the scFv comprises an amino acid sequence at least 96%, identical to the amino acid sequence of SEQ ID NO:148. In certain embodiments, the second antigen-binding site is present as an scFv, wherein the scFv comprises an amino acid sequence at least 97%, identical to the amino acid sequence of SEQ ID NO:148. In certain embodiments, the second antigen-binding site is present as an scFv, wherein the scFv comprises an amino acid sequence at least 98%, identical to the amino acid sequence of SEQ ID NO:148. In certain embodiments, the second antigen-binding site is present as an scFv, wherein the scFv comprises an amino acid sequence at least 99%, identical to the amino acid sequence of SEQ ID NO:148. In certain embodiments, the second antigen-binding site is present as an scFv, wherein the scFv comprises the amino acid sequence of SEQ ID NO:148.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:167.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:169.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:293.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:171.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:229.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:173.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:175.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:233.

In certain embodiments, the second antigen-binding site comprises (a) a VH that comprises CDR1, CDR2, and CDR3 comprising the amino acid sequences of SEQ ID NOs: 152, 158, and 153, respectively; and (b) a VL that comprises CDR1, CDR2, and CDR3 comprising the amino acid sequences of SEQ ID NOs: 154, 155, and 156, respectively.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:150, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:151. In certain embodiments, the VH comprises CDR1, CDR2, and CDR3 comprising the amino acid sequences of SEQ ID NOs: 152, 158, and 153, respectively. In certain embodiments, the VL comprises CDR1, CDR2, and CDR3 comprising the amino acid sequences of SEQ ID NOs: 154, 155, and 156, respectively. In certain embodiments, the VH comprises a substitution of glutamic acid (E) at position 1, according to the Kabat numbering scheme. Accordingly, in some embodiments, the VH comprises the amino acid sequence of SEQ ID NO:157.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:159, SEQ ID NO:221 or SEQ ID NO:160.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:221.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:159.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:160.

In certain embodiments, the second antigen-binding site comprises (a) a VH that comprises CDR1, CDR2, and CDR3 comprising the amino acid sequences of SEQ ID NOs: 163, 139, and 164, respectively; and (b) a VL that comprises CDR1, CDR2, and CDR3 comprising the amino acid sequences of SEQ ID NOs: 165, 142, and 143, respectively.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:161, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:162. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 165, 142, and 143, respectively.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:176, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:177. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 178, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:179.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:242, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:177. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 178, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:243.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:245, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:177. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 178, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:246.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:161, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:177. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 178, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:202.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:180, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:177. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 178, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:181.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:247, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:177. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 178, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:248.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:182, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:177. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 178, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:183.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:250, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:177. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 178, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:251.

In certain embodiments, the second antigen-binding site comprises (a) a VH that comprises CDR1, CDR2, and CDR3 comprising the amino acid sequences of SEQ ID NOs: 186, 187, and 188, respectively; and (b) a VL that comprises CDR1, CDR2, and CDR3 comprising the amino acid sequences of SEQ ID NOs: 189, 190, and 143, respectively.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:184, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:185. In certain embodiments, the VH comprises CDR1, CDR2, and CDR3 comprising the amino acid sequences of SEQ ID NOs: 186, 187, 188, respectively. In certain embodiments, the VL comprises CDR1, CDR2, and CDR3 comprising the amino acid sequences of SEQ ID NOs: 189, 190, and 143, respectively.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:191, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:192. In certain embodiments, the VH comprises CDR1, CDR2, and CDR3 comprising the amino acid sequences of SEQ ID NOs: 186, 187, and 188, respectively. In certain embodiments, the VL comprises CDR1, CDR2, and CDR3 comprising the amino acid sequences of SEQ ID NOs: 189, 190, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:193 or SEQ ID NO:194.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:294 or SEQ ID NO:295.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:195, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:196. In certain embodiments, the VH comprises CDR1, CDR2, and CDR3 comprising the amino acid sequences of SEQ ID NOs: 186, 187, and 188, respectively. In certain embodiments, the VL comprises CDR1, CDR2, and CDR3 comprising the amino acid sequences of SEQ ID NOs: 189, 190, and 143, respectively.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:266, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:177. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 178, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:271.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:266, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:162. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 165, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:272.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:266, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:267. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 268, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:273.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:269, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:177. In certain embodiments, the VH comprises CDR1, CDR2, and CDR3 comprising the amino acid sequences of SEQ ID NOs: 163, 139, and 270, respectively. In certain embodiments, the VL comprises CDR1, CDR2, and CDR3 comprising the amino acid sequences of SEQ ID NOs: 178, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:274.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:269, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:162. In certain embodiments, the VH comprises CDR1, CDR2, and CDR3 comprising the amino acid sequences of SEQ ID NOs: 163, 139, and 270, respectively. In certain embodiments, the VL comprises CDR1, CDR2, and CDR3 comprising the amino acid sequences of SEQ ID NOs: 165, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:275.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:269, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:267. In certain embodiments, the VH comprises CDR1, CDR2, and CDR3 comprising the amino acid sequences of SEQ ID NOs: 163, 139, and 270, respectively. In certain embodiments, the VL comprises CDR1, CDR2, and CDR3 comprising the amino acid sequences of SEQ ID NOs: 268, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:276.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:161, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:177. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 178, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:277.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:161, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:162. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 165, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:278.

In certain embodiments, the second antigen-binding site that binds 5T4 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:161, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:267. In certain embodiments, the VH comprises CDR1, CDR2, and CDR3 comprising the amino acid sequences of SEQ ID NOs: 163, 139, and 164, respectively. In certain embodiments, the VL comprises CDR1, CDR2, and CDR3 comprising the amino acid sequences of SEQ ID NOs: 268, 142, and 143, respectively.

In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:279.

In certain embodiments, the second-antigen binding site comprises a VH comprising CDR1, CDR2, and CDR3 comprising the amino acid sequences of SEQ ID NOs:138, 139, and 140, respectively, and a VL comprising CDR1, CDR2, and CDR3 comprising the amino acid sequences of SEQ ID NOs:141, 142, and 143, respectively, wherein the antigen-binding site binds 5T4 within an LRR1 domain. CDR sequences are recognized as features driving antigen-binding properties, accordingly, one of skill in the art understands that an antigen-binding site comprising the same CDRs is expected to exhibit similar antigen-binding properties. In some embodiments, the antigen-binding site that comprises a VH comprising CDR1, CDR2, and CDR3 comprising the amino acid sequences of SEQ ID NOs: 138, 139, and 140, respectively, and a VL comprising CDR1, CDR2, and CDR3 comprising the amino acid sequences of SEQ ID NOs: 141, 142, and 143, respectively, is a human antigen-binding site. In some embodiments, the antigen-binding site that comprises a VH comprising CDR1, CDR2, and CDR3 comprising the amino acid sequences of SEQ ID NOs: 138, 139, and 140, respectively, and a VL comprising CDR1, CDR2, and CDR3 comprising the amino acid sequences of SEQ ID NOs: 141, 142, and 143, respectively, is a murine antigen-binding site.

In certain embodiments, the second-antigen binding site comprises a VH comprising CDR1, CDR2, and CDR3 comprising the amino acid sequences of SEQ ID NOs: 163, 139, and 164, respectively, and a VL comprising CDR1, CDR2, and CDR3 comprising the amino acid sequences of SEQ ID NOs: 165, 142, and 143, respectively, wherein the antigen-binding site binds 5T4 within an LRR1 domain. CDR sequences are recognized as features driving antigen-binding properties, accordingly, one of skill in the art understands that an antigen-binding site comprising the same CDRs is expected to exhibit similar antigen-binding properties. In some embodiments, the antigen-binding site that comprises a VH comprising CDR1, CDR2, and CDR3 comprising the amino acid sequences of SEQ ID NOs: 163, 139, and 164, respectively, and a VL comprising CDR1, CDR2, and CDR3 comprising the amino acid sequences of SEQ ID NOs: 165, 142, and 143, respectively, is a human antigen-binding site. In some embodiments, the antigen-binding site that comprises a VH comprising CDR1, CDR2, and CDR3 comprising the amino acid sequences of SEQ ID NOs: 163, 139, and 164, respectively, and a VL comprising CDR1, CDR2, and CDR3 comprising the amino acid sequences of SEQ ID NOs: 165, 142, and 143, respectively, is a murine antigen-binding site.

In certain embodiments, the second-antigen binding site comprises a VH comprising CDR1, CDR2, and CDR3 comprising the amino acid sequences of SEQ ID NOs: 186, 187, and 188, respectively, and a VL comprising CDR1, CDR2, and CDR3 comprising the amino acid sequences of SEQ ID NOs: 189, 190, and 143, respectively, wherein the antigen-binding site binds 5T4 within the LRR2 domain. CDR sequences are recognized as features driving antigen-binding properties, accordingly, one of skill in the art understands that an antigen-binding site comprising the same CDRs is expected to exhibit similar antigen-binding properties. In some embodiments, the antigen-binding site that comprises a VH comprising CDR1, CDR2, and CDR3 comprising the amino acid sequences of SEQ ID NOs: 186, 187, and 188, respectively, and a VL comprising CDR1, CDR2, and CDR3 comprising the amino acid sequences of SEQ ID NOs: 189, 190, and 143, respectively, is a human antigen-binding site. In some embodiments, the antigen-binding site that comprises a VH comprising CDR1, CDR2, and CDR3 comprising the amino acid sequences of SEQ ID NOs: 186, 187, and 188, respectively, and a VL comprising CDR1, CDR2, and CDR3 comprising the amino acid sequences of SEQ ID NOs: 189, 190, and 143, respectively, is a murine antigen-binding site.

In certain embodiments, the second antigen-binding site that binds 5T4 is an scFv. For example, in certain embodiments, the second antigen-binding site comprises the amino acid sequence of SEQ ID NO: 148, 149, 159, 160, 167, 169, 171, 173, 175, 179, 181, 183, 193, 194, 202, 221, 229, 233, 243, 246, 248, 251, 264, 265, 271, 272, 273, 274, 275, 276, 277, 278, 279, 293, 294, 295, 296, or 297. In certain embodiments, the second antigen-binding site comprises the amino acid sequence of SEQ ID NO: 148 or 149. In certain embodiments, the second antigen-binding site comprises the amino acid sequence of SEQ ID NO:148.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences, respectively:

SEQ ID NOs: 81, 82, 112, 86, 77 and 87; or

SEQ ID NOs: 81, 82, 97, 86, 77 and 87; and

the second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences, respectively:

SEQ ID NOs: 138, 139, 140, 141, 142 and 143.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively:

SEQ ID NOs: 81, 82, 112, 86, 77 and 87;

SEQ ID NOs: 81, 82, 84, 86, 77 and 87;

SEQ ID NOs: 81, 82, 97, 86, 77 and 87;

SEQ ID NOs: 81, 82, 100, 86, 77 and 87;

SEQ ID NOs: 81, 82, 103, 86, 77 and 87;

SEQ ID NOs: 81, 82, 106, 86, 77 and 87; or

SEQ ID NOs: 81, 82, 109, 86, 77 and 87; and

the second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Kabat numbering scheme), respectively:

SEQ ID NOs: 472, 473, 140, 141, 142 and 143;

SEQ ID NOs: 472, 474, 140, 141, 142 and 143;

SEQ ID NOs: 472, 474, 140, 290, 142 and 143;

SEQ ID NOs: 472, 474, 140, 292, 142 and 143;

SEQ ID NOs: 472, 475, 140, 141, 142 and 143; or

SEQ ID NOs: 472, 475, 140, 541, 142 and 143.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively:

SEQ ID NOs: 81, 82, 112, 86, 77 and 87; or

SEQ ID NOs: 81, 82, 97, 86, 77 and 87; and

the second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Kabat numbering scheme), respectively:

SEQ ID NOs: 472, 474, 140, 141, 142 and 143.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 81, 82, 112, 86, 77 and 87; and

the second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Kabat numbering scheme), respectively:

SEQ ID NOs: 472, 475, 140, 141, 142 and 143; or

SEQ ID NOs: 472, 475, 140, 541, 142 and 143.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 81, 82, 97, 86, 77 and 87; and the second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Kabat numbering scheme), respectively: SEQ ID NOs: 472, 474, 140, 141, 142 and 143.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Chothia numbering scheme), respectively:

SEQ ID NOs: 381, 390, 391, 392, 385 and 393;

SEQ ID NOs: 381, 390, 394, 392, 385 and 393;

SEQ ID NOs: 381, 390, 395, 392, 385 and 393;

SEQ ID NOs: 381, 390, 396, 392, 385 and 393;

SEQ ID NOs: 381, 390, 397, 392, 385 and 393;

SEQ ID NOs: 381, 390, 398, 392, 385 and 393; or

SEQ ID NOs: 381, 390, 399, 392, 385 and 393; and

the second first antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Chothia numbering scheme), respectively:

SEQ ID NOs: 138, 482, 483, 484, 485 and 486;

SEQ ID NOs: 152, 487, 488, 489, 490 and 491;

SEQ ID NOs: 163, 482, 492, 493, 485 and 486;

SEQ ID NOs: 163, 482, 492, 494, 485 and 486; or

SEQ ID NOs: 186, 495, 496, 497, 498 and 486.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Chothia numbering scheme), respectively:

SEQ ID NOs: 381, 390, 391, 392, 385 and 393; or

SEQ ID NOs: 381, 390, 395, 392, 385 and 393; and

the second first antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Chothia numbering scheme), respectively:

SEQ ID NOs: 138, 482, 483, 484, 485 and 486.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Chothia numbering scheme), respectively: SEQ ID NOs: 381, 390, 395, 392, 385 and 393; and

the second first antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Chothia numbering scheme), respectively: SEQ ID NOs: 138, 482, 483, 484, 485 and 486.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to IMGT numbering scheme), respectively:

SEQ ID NOs: 422, 423, 111, 424, 385 and 87;

SEQ ID NOs: 422, 423, 83, 424, 385 and 87;

SEQ ID NOs: 422, 423, 96, 424, 385 and 87;

SEQ ID NOs: 422, 423, 99, 424, 385 and 87;

SEQ ID NOs: 422, 423, 102, 424, 385 and 87;

SEQ ID NOs: 422, 423, 105, 424, 385 and 87; or

SEQ ID NOs: 422, 423, 108, 424, 385 and 87; and

the second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to IMGT numbering scheme), respectively:

SEQ ID NOs: 499, 500, 501, 502, 485 and 143;

SEQ ID NOs: 503, 504, 505, 506, 490 and 156;

SEQ ID NOs: 507, 508, 509, 510, 485 and 143;

SEQ ID NOs: 507, 508, 511, 510, 485 and 143; or

SEQ ID NOs: 512, 513, 514, 515, 498 and 143.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to IMGT numbering scheme), respectively:

SEQ ID NOs: 422, 423, 111, 424, 385 and 87; or

SEQ ID NOs: 422, 423, 96, 424, 385 and 87; and

the second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to IMGT numbering scheme), respectively:

SEQ ID NOs: 499, 500, 501, 502, 485 and 143.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to IMGT numbering scheme), respectively: SEQ ID NOs: 422, 423, 96, 424, 385 and 87; and the second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to IMGT numbering scheme), respectively: SEQ ID NOs: 499, 500, 501, 502, 485 and 143.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Honegger numbering scheme), respectively:

SEQ ID NOs: 462, 463, 464, 465, 459 and 393;

SEQ ID NOs: 462, 463, 466, 465, 459 and 393;

SEQ ID NOs: 462, 463, 467, 465, 459 and 393;

SEQ ID NOs: 462, 463, 468, 465, 459 and 393;

SEQ ID NOs: 462, 463, 469, 465, 459 and 393;

SEQ ID NOs: 462, 463, 470, 465, 459 and 393; or

SEQ ID NOs: 462, 463, 471, 465, 459 and 393; and

the second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Honegger numbering scheme), respectively:

SEQ ID NOs: 516, 517, 518, 519, 520 and 486;

SEQ ID NOs: 516, 521, 518, 519, 522 and 486;

SEQ ID NOs: 516, 523, 518, 519, 522 and 486; or

SEQ ID NOs: 516, 523, 518, 519, 542 and 486.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Honegger numbering scheme), respectively:

SEQ ID NOs: 462, 463, 464, 465, 459 and 393; or

SEQ ID NOs: 462, 463, 467, 465, 459 and 393; and

the second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Honegger numbering scheme), respectively:

SEQ ID NOs: 516, 521, 518, 519, 522 and 486.

In various embodiments, the first antigen-binding domain that binds NKG2D (e.g., human NKG2D) comprises a VH-CDR1, a VH-CDR2, a VH-CDR3, a VL-CDR1, a VL-CDR2 and a VL-CDR3 comprising the following amino acid sequences (according to Honegger numbering scheme), respectively: SEQ ID NOs: 462, 463, 467, 465, 459 and 393; and

the second antigen-binding domain that binds 5T4 (e.g., human 5T4) comprises a VH-CDR1, a VH-CDR2, a VH-CDR3, a VL-CDR1, a VL-CDR2 and a VL-CDR3 comprising the following amino acid sequences (according to Honegger numbering scheme), respectively:

SEQ ID NOs: 516, 521, 518, 519, 522 and 486;

SEQ ID NOs: 516, 523, 518, 519, 522 and 486; or

SEQ ID NOs: 516, 523, 518, 519, 542 and 486.

In certain embodiments, the second antigen-binding site that binds to 5T4 comprises a VH comprising a CDR1, a CDR2, and a CDR3 sequence selected from Table 12 and a VL comprising a CDR1, a CDR2, and a CDR3 sequence comprising the amino acid sequences of SEQ ID NOs: 189, 190, and 143, respectively. For example, in certain embodiments, the second antigen-binding site comprises a VH comprising a CDR1, a CDR2, and a CDR3 sequence selected from the group consisting of: (a) GYTFTSY (SEQ ID NO:186), DSSDSK (SEQ ID NO:187), and GGYLWFAY (SEQ ID NO:188); (b) GYTFGSY (SEQ ID NO:203), DASTEK (SEQ ID NO:204), and GGYLWFQY (SEQ ID NO:205); (c) GYLFTSY (SEQ ID NO:206), SVSDAK (SEQ ID NO:207), and GGYLWFKY (SEQ ID NO:208); (d) GYTFGSY (SEQ ID NO: 203), DARSAK (SEQ ID NO:209), and GGYLWFKY(SEQ ID NO: 208); (e) GYRFTSY (SEQ ID NO:210), DASSAK (SEQ ID NO:211), and GGYLWFKY (SEQ ID NO: 208); (f) GYGFTSY (SEQ ID NO:212), DARTAK (SEQ ID NO:213), and GGYLWYAY (SEQ ID NO:214); (g) GYTFTSY (SEQ ID NO: 186), DASDAK (SEQ ID NO:215), and GGYLWYHY (SEQ ID NO:216); (h) GYTFTSY (SEQ ID NO: 186), DASDAK (SEQ ID NO:215), and GGYLWYSY (SEQ ID NO:217); (i) GYTFTSY (SEQ ID NO: 186), DASDAK (SEQ ID NO:215), and GGYLWYAY (SEQ ID NO:214); (j) GYSFTSY (SEQ ID NO:218), DASDAK (SEQ ID NO:215), and GGYLWFKY (SEQ ID NO: 208); (k) GYTFTSY (SEQ ID NO: 186), DASDAK (SEQ ID NO:215), and GGYLWFKY (SEQ ID NO: 208); and (1) GYGFTSY (SEQ ID NO:212), DARTAK (SEQ ID NO:213), and GGHLWYAY (SEQ ID NO:219). Such second antigen-binding site that binds to 5T4 can be formed by combining ane one of these VHs with a VL comprising a CDR1, a CDR2, and a CDR3 sequence comprising the amino acid sequences of SEQ ID NOs: 189, 190, and 143, respectively.

Alternatively, novel antigen-binding sites that can bind to 5T4 can be identified by screening for binding to the amino acid sequence defined by binding to the amino acid sequence defined by SEQ ID NO:197, a variant thereof, a mature extracellular fragment thereof or a fragment containing a domain of 5T4.

(SEQ ID NO: 197)
MPGGCSRGPAAGDGRLRLARLALVLLGWVSSSSPTSSASSFSSSAPF
LASAVSAQPPLPDQCPALCECSEAARTVKCVNRNLTEVPTDLPAYVR
NLFLTGNQLAVLPAGAFARRPPLAELAALNLSGSRLDEVRAGAFEHL
PSLRQLDLSHNPLADLSPFAFSGSNASVSAPSPLVELILNHIVPPED
ERQNRSFEGMVVAALLAGRALQGLRRLELASNHFLYLPRDVLAQLPS
LRHLDLSNNSLVSLTYVSFRNLTHLESLHLEDNALKVLHNGTLAELQ
GLPHIRVFLDNNPWVCDCHMADMVTWLKETEWQGKDRLTCAYPEKMR
NRVLLELNSADLDCDPILPPSLQTSYVFLGIVLALIGAIELL

It is contemplated that in an scFv, a VH and a VL can be connected by a linker, e.g., (GlyGlyGlyGlySer)₄ i.e. (G₄S)₄ linker (SEQ ID NO:119). A skilled person in the art would appreciate that any of the other disclosed linkers (see, e.g., Table 10) may be used in an scFv having a VH and VL sequence disclosed herein (e.g., in Table 2).

In each of the foregoing embodiments, it is contemplated herein that the scFv, VH and/or VL sequences that bind 5T4 may contain amino acid alterations (e.g., at least 1, 2, 3, 4, 5, or 10 amino acid substitutions, deletions, or additions) in the framework regions of the VH and/or VL without affecting their ability to 5T4. For example, it is contemplated herein that scFv, VH and/or VL sequences that bind 5T4 may contain cysteine heterodimerization mutations, facilitating formation of a disulfide bridge between the VH and VL of the scFv.

In certain embodiments, the second antigen-binding site competes for binding to 5T4 with a corresponding antigen-binding site described above.

In certain embodiments, a multispecific binding protein of the present disclosure specifically binds 5T4 (e.g., human 5T4 or cynomolgus 5T4) with a K_{D} (i.e., dissociation constant) of 25 nM, 20 nM, 15 nM, 10 nM, 9 nM, 8 nM, 7 nM, 6 nM, 5 nM, 4 nM, 3 nM, 2 nM, 1 nM, 0.1 nM or lower, as measured using standard binding assays, for example, surface plasmon resonance (SPR) (e.g., using the method described in Example 1 infra) or bio-layer interferometry (BLI). In certain embodiments, the multispecific binding protein as disclosed herein specifically binds 5T4 with a K_{D} less than 9 nM. In certain embodiments, the multispecific binding protein as disclosed herein specifically binds 5T4 with a K_{D} less than 8 nM. In certain embodiments, the multispecific binding protein as disclosed herein specifically binds 5T4 with a K_{D} less than 7 nM. In certain embodiments, the multispecific binding protein as disclosed herein specifically binds 5T4 with a K_{D} less than 6 nM. In certain embodiments, the multispecific binding protein as disclosed herein specifically binds 5T4 with a K_{D} less than 5 nM. In certain embodiments, a multispecific binding protein of the present disclosure specifically binds 5T4 (e.g., human 5T4 or cynomolgus 5T4) with a K_{d}(i.e., off-rate, also called Koff) equal to or lower than 1×10⁻⁵, 9×10⁻⁴, 8×10⁻⁴, 7×10⁻⁴, 6×10⁻⁴, 5×10⁻⁴, 4×10⁻⁴, 3×10⁻⁴, 2×10⁻⁴, 1×10⁻⁴, 1×10⁻³, 5×10³, 0.01, 0.02, or 0.05 l/s, as measured by SPR (e.g., using the method described in Example 1 infra) or by BLI.

### Fc Domain

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction. Accordingly, in certain embodiments, the antibody Fc domain or the portion thereof comprises a hinge and a CH2 domain.

In some embodiments, a multispecific binding protein described herein includes the VH or VL of one or more antigen binding sites fused to the N-terminus of an antibody Fc domain polypeptide or portion thereof. Such antigen binding sites can include the VH or the VL of the anti-NKG2D antibody or the anti-5T4 antibody as described herein. Accordingly, in some embodiments, the VH or the VL of the anti-NKG2D antibody is fused to the N-terminus of an antibody Fc domain polypeptide or portion thereof sufficient to bind CD16. In some embodiments, the VH or the VL of the anti-5T4 antibody is fused to the N-terminus of an antibody Fc domain polypeptide or portion thereof sufficient to bind CD16. In some embodiments, the VH or the VL of the anti-NKG2D antibody is fused to the N-terminus of a first antibody Fc domain polypeptide or portion thereof sufficient to bind CD16, and the VH or the VL of the anti-5T4 antibody is fused to the N-terminus of a second antibody Fc domain polypeptide or portion thereof sufficient to bind CD16. In some embodiments, the VH of the anti-NKG2D antibody is fused to the N-terminus of the first antibody Fc domain polypeptide or portion thereof sufficient to bind CD16, and the VH of the anti-5T4 antibody is fused to the N-terminus of the second antibody Fc domain polypeptide or portion thereof sufficient to bind CD16.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, U.S. Ser. No. 13/642,253, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that these conventions consist of nonsequential numbering in specific regions of an immunoglobulin sequence, enabling a normalized reference to conserved positions in immunoglobulin families. Accordingly, the positions of any given immunoglobulin as defined by the EU index or by the Kabat numbering scheme will not necessarily correspond to its sequential sequence.

With knowledge of the residue number according to Kabat or EU index numbering, one of ordinary skill can apply the teachings of the art to identify amino acid sequence modifications within the present disclosure, according to any commonly used numbering convention. It is understood that the SEQ ID NOs provide sequential numbering of amino acids within a given polypeptide and, thus, may not conform to the corresponding amino acid numbers as provided by Kabat or EU index.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain described in the application can optionally be coupled to an amino acid sequence at least 90% (e.g., at least 91%, at least 92%, at least 93%, at least 94%, at least 95%, at least 96%, at least 97%, at least 98%, at least 99% or 100%) identical to an antibody constant region, such as an IgG constant region including hinge, CH2 and CH3 domains with or without CH1 domain. In some embodiments, the amino acid sequence of the constant region is at least 90% (e.g., at least 91%, at least 92%, at least 93%, at least 94%, at least 95%, at least 96%, at least 97%, at least 98%, at least 99% or 100%) identical to a human antibody constant region, such as a human IgG1 constant region, an IgG2 constant region, IgG3 constant region, or IgG4 constant region. In one embodiment, the antibody Fc domain or a portion thereof sufficient to bind CD16 comprises an amino acid sequence at least 90% (e.g., at least 91%, at least 92%, at least 93%, at least 94%, at least 95%, at least 96%, at least 97%, at least 98%, at least 99%, or 100%) identical to wild-type human IgG1 Fc sequence set forth below;

(SEQ ID NO: 118)
DKTHTCPPCPAPELLGGPSVELEPPKPKDTLMISRTPEVTCVVVDVSHE
DPEVKENWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPG.

In some embodiments, the antibody Fc domain or a portion thereof comprises an amino acid sequence at least 95% identical to the amino acid sequence of SEQ ID NO:118. In some embodiments, the antibody Fc domain or a portion thereof comprises an amino acid sequence at least 96% identical to the amino acid sequence of SEQ ID NO:118. In some embodiments, the antibody Fc domain or a portion thereof comprises an amino acid sequence at least 97% identical to the amino acid sequence of SEQ ID NO:118. In some embodiments, the antibody Fc domain or a portion thereof comprises an amino acid sequence at least 98% identical to the amino acid sequence of SEQ ID NO:118. In some embodiments, the antibody Fc domain or a portion thereof comprises an amino acid sequence at least 99% identical to the amino acid sequence of SEQ ID NO:118. In some embodiments, the antibody Fc domain or a portion thereof comprises the amino acid sequence of SEQ ID NO:118.

In some other embodiments, the amino acid sequence of the constant region is at least 90% (e.g., at least 91%, at least 92%, at least 93%, at least 94%, at least 95%, at least 96%, at least 97%, at least 98%, at least 99% or 100%) identical to an antibody constant region from another mammal, such as rabbit, dog, cat, mouse, or horse.

In some embodiments, the multispecific binding protein described herein comprises an Fc domain or portion thereof that is sufficient to bind to CD16 (e.g., human CD16). In some embodiments, the antibody constant domain linked to the scFv or the Fab fragment is able to bind to CD16 (e.g., human CD16). In some embodiments, the protein incorporates a portion of an antibody Fc domain (for example, a portion of an antibody Fc domain sufficient to bind CD16 (e.g., human CD16)), wherein the antibody Fc domain comprises a hinge and a CH2 domain (for example, a hinge and a CH2 domain of a human IgG1 antibody), and/or amino acid sequences at least 90% (e.g., at least 91%, at least 92%, at least 93%, at least 94%, at least 95%, at least 96%, at least 97%, at least 98%, at least 99% or 100%) identical to amino acid sequence 234-332 of a human IgG antibody. In some embodiments, the CD16 is human CD16. In some embodiments, the human CD16 is human CD16a (FcγRIIIa).

One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, F405L, Y407A, Y407I, Y407V, K409F, K409W, K409D, K409R, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

Alternatively, amino acid substitutions could be selected from the following sets of substitutions shown in Table 3.

TABLE 3

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 4.

TABLE 4

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following sets of substitutions shown in Table 5.

TABLE 5

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 6.

TABLE 6

| First Polypeptide | Second Polypeptide |
|---|---|
| L351Y, D399R, D399K, S400K, | T366V, T366I, T366L, T366M, |
| S400R, Y407A, Y407I, Y407V | N390D, N390E, K392L, K392M, |
| | K392V, K392F K392D, K392E, |
| | K409F, K409W, T411D and |
| | T411E |

Alternatively, at least one amino acid substitution could be selected from the following sets of substitutions in Table 7, where the position(s) indicated in the First Polypeptide column is replaced by any known negatively-charged amino acid, and the position(s) indicated in the Second Polypeptide Column is replaced by any known positively-charged amino acid.

TABLE 7

| First Polypeptide | Second Polypeptide |
|---|---|
| K392, K370, K409, or K439 | D399, E356, or E357 |

Alternatively, at least one amino acid substitution could be selected from the following set in Table 8, where the position(s) indicated in the First Polypeptide column is replaced by any known positively-charged amino acid, and the position(s) indicated in the Second Polypeptide Column is replaced by any known negatively-charged amino acid.

TABLE 8

| First Polypeptide | Second Polypeptide |
|---|---|
| D399, E356, or E357 | K409, K439, K370, or K392 |

Alternatively, amino acid substitutions could be selected from the following sets in Table 9.

TABLE 9

| First Polypeptide | Second Polypeptide |
|---|---|
| T350V, L351Y, F405A, and | T350V, T366L, K392L, and |
| Y407V | T394W |

Alternatively, or in addition, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

In some embodiments, the amino acid sequence of one polypeptide chain of the antibody constant region differs from the amino acid sequence of an IgG1 (e.g., human IgG1) constant region at position T366, and wherein the amino acid sequence of the other polypeptide chain of the antibody constant region differs from the amino acid sequence of an IgG1 (e.g., human IgG1) constant region at one or more positions selected from the group consisting of T366, L368 and Y407.

In some embodiments, the amino acid sequence of one polypeptide chain of the antibody constant region differs from the amino acid sequence of an IgG1 (e.g., human IgG1) constant region at one or more positions selected from the group consisting of T366, L368 and Y407, and wherein the amino acid sequence of the other polypeptide chain of the antibody constant region differs from the amino acid sequence of an IgG1 (e.g., human IgG1) constant region at position T366.

In some embodiments, the amino acid sequence of one polypeptide chain of the antibody constant region differs from the amino acid sequence of an IgG1 (e.g., human IgG1) constant region at one or more positions selected from the group consisting of E357, K360, Q362, S364, L368, K370, T394, D401, F405, and T411 and wherein the amino acid sequence of the other polypeptide chain of the antibody constant region differs from the amino acid sequence of an IgG1 (e.g., human IgG1) constant region at one or more positions selected from the group consisting of Y349, E357, S364, L368, K370, T394, D401, F405 and T411.

In some embodiments, the amino acid sequence of one polypeptide chain of the antibody constant region differs from the amino acid sequence of an IgG1 (e.g., human IgG1) constant region at one or more positions selected from the group consisting of Y349, E357, S364, L368, K370, T394, D401, F405 and T411 and wherein the amino acid sequence of the other polypeptide chain of the antibody constant region differs from the amino acid sequence of an IgG1 (e.g., human IgG1) constant region at one or more positions selected from the group consisting of E357, K360, Q362, S364, L368, K370, T394, D401, F405, and T411.

In some embodiments, the amino acid sequence of one polypeptide chain of the antibody constant region differs from the amino acid sequence of an IgG1 (e.g., human IgG1) constant region at one or more positions selected from the group consisting of L351, D399, S400 and Y407 and wherein the amino acid sequence of the other polypeptide chain of the antibody constant region differs from the amino acid sequence of an IgG1 (e.g., human IgG1) constant region at one or more positions selected from the group consisting of T366, N390, K392, K409 and T411.

In some embodiments, the amino acid sequence of one polypeptide chain of the antibody constant region differs from the amino acid sequence of an IgG1 (e.g., human IgG1) constant region at one or more positions selected from the group consisting of T366, N390, K392, K409 and T411 and wherein the amino acid sequence of the other polypeptide chain of the antibody constant region differs from the amino acid sequence of an IgG1 (e.g., human IgG1) constant region at one or more positions selected from the group consisting of L351, D399, S400 and Y407.

In some embodiments, the amino acid sequence of one polypeptide chain of the antibody constant region differs from the amino acid sequence of an IgG1 (e.g., human IgG1) constant region at one or more positions selected from the group consisting of Q347, Y349, K360, and K409, and wherein the amino acid sequence of the other polypeptide chain of the antibody constant region differs from the amino acid sequence of an IgG1 (e.g., human IgG1) constant region at one or more positions selected from the group consisting of Q347, E357, D399 and F405.

In some embodiments, the amino acid sequence of one polypeptide chain of the antibody constant region differs from the amino acid sequence of an IgG1 (e.g., human IgG1) constant region at one or more positions selected from the group consisting of Q347, E357, D399 and F405, and wherein the amino acid sequence of the other polypeptide chain of the antibody constant region differs from the amino acid sequence of an IgG1 (e.g., human IgG1) constant region at one or more positions selected from the group consisting of Y349, K360, Q347 and K409.

In some embodiments, the amino acid sequence of one polypeptide chain of the antibody constant region differs from the amino acid sequence of an IgG1 (e.g., human IgG1) constant region at one or more positions selected from the group consisting of K370, K392, K409 and K439, and wherein the amino acid sequence of the other polypeptide chain of the antibody constant region differs from the amino acid sequence of an IgG1 (e.g., human IgG1) constant region at one or more positions selected from the group consisting of D356, E357 and D399.

In some embodiments, the amino acid sequence of one polypeptide chain of the antibody constant region differs from the amino acid sequence of an IgG1 (e.g., human IgG1) constant region at one or more positions selected from the group consisting of D356, E357 and D399, and wherein the amino acid sequence of the other polypeptide chain of the antibody constant region differs from the amino acid sequence of an IgG1 (e.g., human IgG1) constant region at one or more positions selected from the group consisting of K370, K392, K409 and K439.

In some embodiments, the amino acid sequence of one polypeptide chain of the antibody constant region differs from the amino acid sequence of an IgG1 (e.g., human IgG1) constant region at one or more positions selected from the group consisting of L351, E356, T366 and D399, and wherein the amino acid sequence of the other polypeptide chain of the antibody constant region differs from the amino acid sequence of an IgG1 (e.g., human IgG1) constant region at one or more positions selected from the group consisting of Y349, L351, L368, K392 and K409.

In some embodiments, the amino acid sequence of one polypeptide chain of the antibody constant region differs from the amino acid sequence of an IgG1 (e.g., human IgG1) constant region at one or more positions selected from the group consisting of Y349, L351, L368, K392 and K409, and wherein the amino acid sequence of the other polypeptide chain of the antibody constant region differs from the amino acid sequence of an IgG1 (e.g., human IgG1) constant region at one or more positions selected from the group consisting of L351, E356, T366 and D399.

In some embodiments, the amino acid sequence of one polypeptide chain of the antibody constant region differs from the amino acid sequence of an IgG1 (e.g., human IgG1) constant region by an S354C substitution and wherein the amino acid sequence of the other polypeptide chain of the antibody constant region differs from the amino acid sequence of an IgG1 (e.g., human IgG1) constant region by a Y349C substitution. In some embodiments, the one polypeptide chain comprising the S354C substitution is fused to a VH of an anti-NKG2D antibody described herein. In some embodiments, the one polypeptide chain comprising the Y349C substitution is fused to a VH of an anti-5T4 antibody described herein. Accordingly, in some embodiments, the VH of the anti-NKG2D antibody is fused to the N-terminus of an Fc domain polypeptide comprising a S354C substitution, and the VH of the anti-5T4 antibody is fused to the N-terminus of an Fc domain polypeptide comprising Y349C substitution.

In some embodiments, the amino acid sequence of one polypeptide chain of the antibody constant region differs from the amino acid sequence of an IgG1 (e.g., human IgG1) constant region by a Y349C substitution and wherein the amino acid sequence of the other polypeptide chain of the antibody constant region differs from the amino acid sequence of an IgG1 (e.g., human IgG1) constant region by an S354C substitution. In some embodiments, the one polypeptide chain comprising the Y349C substitution is fused to a VH of an anti-NKG2D antibody described herein. In some embodiments, the one polypeptide chain comprising the S354C substitution is fused to a VH of an anti-5T4 antibody described herein. Accordingly, in some embodiments, the VH of the anti-NKG2D antibody is fused to the N-terminus of an Fc domain polypeptide comprising a Y349C substitution, and the VH of the anti-5T4 antibody is fused to the N-terminus of an Fc domain polypeptide comprising S354C substitution.

In some embodiments, the amino acid sequence of one polypeptide chain of the antibody constant region differs from the amino acid sequence of an IgG1 (e.g., human IgG1) constant region by K360E and K409W substitutions and wherein the amino acid sequence of the other polypeptide chain of the antibody constant region differs from the amino acid sequence of an IgG1 (e.g., human IgG1) constant region by Q347R, D399V and F405T substitutions. In some embodiments, the one polypeptide chain comprising the K360E and K409W substitutions is fused to a VH of an anti-NKG2D antibody described herein. In some embodiments, the one polypeptide chain comprising the Q347R, D399V and F405T substitutions is fused to a VH of an anti-5T4 antibody described herein. Accordingly, in some embodiments, the VH of the anti-NKG2D antibody is fused to the N-terminus of an Fc domain polypeptide comprising K360E and K409W substitutions, and the VH of the anti-5T4 antibody is fused to the N-terminus of an Fc domain polypeptide comprising Q347R, D399V and F405T substitutions.

In some embodiments, the amino acid sequence of one polypeptide chain of the antibody constant region differs from the amino acid sequence of an IgG1 (e.g., human IgG1) constant region by Q347R, D399V and F405T substitutions and wherein the amino acid sequence of the other polypeptide chain of the antibody constant region differs from the amino acid sequence of an IgG1 (e.g., human IgG1) constant region by K360E and K409W substitutions.

In some embodiments, the amino acid sequence of one polypeptide chain of the antibody constant region differs from the amino acid sequence of an IgG1 (e.g., human IgG1) constant region by a T366W substitutions and wherein the amino acid sequence of the other polypeptide chain of the antibody constant region differs from the amino acid sequence of an IgG1 (e.g., human IgG1) constant region by T366S, T368A, and Y407V substitutions.

In some embodiments, the amino acid sequence of one polypeptide chain of the antibody constant region differs from the amino acid sequence of an IgG1 (e.g., human IgG1) constant region by T366S, T368A, and Y407V substitutions and wherein the amino acid sequence of the other polypeptide chain of the antibody constant region differs from the amino acid sequence of an IgG1 (e.g., human IgG1) constant region by a T366W substitution.

In some embodiments, the amino acid sequence of one polypeptide chain of the antibody constant region differs from the amino acid sequence of an IgG1 (e.g., human IgG1) constant region by T350V, L351Y, F405A, and Y407V substitutions and wherein the amino acid sequence of the other polypeptide chain of the antibody constant region differs from the amino acid sequence of an IgG1 (e.g., human IgG1) constant region by T350V, T366L, K392L, and T394W substitutions.

In some embodiments, the amino acid sequence of one polypeptide chain of the antibody constant region differs from the amino acid sequence of an IgG1 (e.g., human IgG1) constant region by T350V, T366L, K392L, and T394W substitutions and wherein the amino acid sequence of the other polypeptide chain of the antibody constant region differs from the amino acid sequence of an IgG1 (e.g., human IgG1) constant region by T350V, L351Y, F405A, and Y407V substitutions.

In some embodiments, the amino acid sequence of one polypeptide chain of the antibody constant region differs from the amino acid sequence of an IgG1 (e.g., human IgG1) constant region by an F405L substitution and wherein the amino acid sequence of the other polypeptide chain of the antibody constant region differs from the amino acid sequence of an IgG1 (e.g., human IgG1) constant region by a K409R substitution.

### Exemplary Multispecific Binding Proteins

Listed below are examples of TriNKETs® (Tri-specific NK cell Engager Therapy) comprising an antigen-binding site that binds 5T4 and an antigen-binding site that binds NKG2D each linked to an antibody constant region, wherein the antibody constant regions include mutations that enable heterodimerization of two Fc chains.

Exemplary 5T4-targeting TriNKETs® are contemplated in the F3′, F4, and 2-Fab formats. As described above, in the F3′ format, the antigen-binding site that binds 5T4 is an scFv and the antigen-binding site that binds NKG2D is a Fab. In the F4 format, the antigen binding-sites that bind 5T4 are Fab fragments and the antigen-binding site that binds NKG2D is an scFv. In each TriNKET®, the scFv may comprise substitution of Cys in the VH and VL regions, facilitating formation of a disulfide bridge between the VH and VL of the scFv. In the 2-Fab format, both the antigen-binding site that binds 5T4 and the antigen-binding site that binds NKG2D are Fabs.

The VH and VL of an scFv can be connected via a linker, e.g., a peptide linker. In certain embodiments, the peptide linker is a flexible linker. Regarding the amino acid composition of the linker, peptides are selected with properties that confer flexibility, do not interfere with the structure and function of the other domains of the proteins described in the present application, and resist cleavage from proteases. For example, glycine and serine residues generally provide protease resistance. In certain embodiments, the VL is linked N-terminal or C-terminal to the VH via a (GlyGlyGlyGlySer)₄ ((G₄S)₄) linker (SEQ ID NO:119).

The length of the linker (e.g., flexible linker) can be “short,” e.g., 0, 1, 2, 3, 4, 5, 6, 7, 8, 9, 10, 11 or 12 amino acid residues, or “long,” e.g., at least 13 amino acid residues. In certain embodiments, the linker is 10-50, 10-40, 10-30, 10-25, 10-20, 15-50, 15-40, 15-30, 15-25, 15-20, 20-50, 20-40, 20-30, or 20-25 amino acid residues in length.

In certain embodiments, the linker comprises or consists of a (GS). (SEQ ID NO:120), (GGS)ₙ (SEQ ID NO:121), (GGGS)ₙ (SEQ ID NO:122), (GGSG)ₙ (SEQ ID NO:123), (GGSGG)ₙ (SEQ ID NO:124), and (GGGGS)ₙ (SEQ ID NO:125) sequence, wherein n is 1, 2, 3, 4, 5, 6, 7, 8, 9, 10, 11, 12, 13, 14, 15, 16, 17, 18, 19, or 20. In certain embodiments, the linker comprises or consists of an amino acid sequence selected from SEQ ID NO:119, SEQ ID NOs:119, 126-135 and SEQ ID NOs: 126-134, as listed in Table 10.

TABLE 10

| SEQ ID | Amino Acid Sequence |
|---|---|
| SEQ ID NO: 126 | GSGSGSGSGSGSGSGSGSGS |
| SEQ ID NO: 127 | GGSGGSGGSGGSGGSGGSGGSGGSGGSGGS |
| SEQ ID NO: 128 | GGGSGGGSGGGSGGGSGGGSGGGSGGGSGGGSGGGSGGGS |
| SEQ ID NO: 129 | GGSGGGSGGGSGGGSGGGSGGGSGGGSGGGSGGGSGGGSG |
| SEQ ID NO: 130 | GGSGGGGSGGGGSGGGGSGGGGSGGGGSGGGGSGGGGSGGGGSG |
| | GGGSGG |
| SEQ ID NO: 131 | GGGGSGGGGSGGGGSGGGGSGGGGSGGGGSGGGGSGGGGSGGGG |
| | SGGGGS |
| SEQ ID NO: 119 | GGGGSGGGGSGGGGSGGGGS |
| SEQ ID NO: 132 | GGGGSGGGGSGGGGS |
| SEQ ID NO: 133 | GGGGSGGGGSGGGGSGGGGSGGGGSGGGGSGGGGSGGGGSGGGG |
| | SGGGGSGGGGSGGGGSGGGGSGGGGSGGGGSGGGGSGGGGSGGG |
| | GSGGGGSGGGGS |
| SEQ ID NO: 134 | GGSGGGGSGGGGSGGGGSGGGGSGGGGSGGGGSGGGGSGGGGSG |
| | GGGSGGGGSGGGGSGGGGSGGGGSGGGGSGGGGSGGGGSGGGGS |
| | GGGGSGGGGSGG |
| SEQ ID NO: 135 | SGSGGGGS |

In the F3′-TriNKETs®, the 5T4 binding scFv is linked to the N-terminus of an Fc via an Ala-Ser or Gly-Ser linker. The Ala-Ser or Gly-Ser linker is included at the elbow hinge region sequence to balance between flexibility and optimal geometry. In certain embodiments, an additional amino acid sequence Thr-Lys-Gly can be added N-terminal or C-terminal to the Ala-Ser or Gly-Ser sequence at the hinge. In the F4 TriNKETs®, the NKG2D-binding scFv is linked to the C-terminus of an Fc via a short linker comprising the amino acid sequence SGSGGGGS (SEQ ID NO:135).

As used herein to describe these exemplary TriNKETs®, an Fc includes an antibody hinge, CH2, and CH3. In each exemplary TriNKET®, the Fc domain linked to an scFv comprises the mutations of Q347R, D399V, and F405T, and the Fc domain linked to a Fab comprises matching mutations K360E and K409W for forming a heterodimer. The Fc domain linked to the scFv further includes an S354C substitution in the CH3 domain, which forms a disulfide bond with a Y349C substitution on the Fc linked to the Fab. These substitutions are bold in the sequences described in this subsection. In certain embodiments, for each of the exemplary TriNKETs® described below, the Fc domain linked to an scFv comprises the mutations of K360E and K409W, and the Fc domain linked to a Fab comprises matching mutations Q347R, D399V, and F405T for forming a heterodimer. In these embodiments, the Fc domain linked to the scFv further includes an Y349C substitution in the CH3 domain, which forms a disulfide bond with a S354C substitution on the Fc linked to the Fab.

For example, a TriNKET® described in the present disclosure is AB1310/AB1783-TriNKET®. AB1310/AB1783-TriNKET® includes (a) a 5T4-binding scFv sequence comprising the VH and VL sequences of AB1002 described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1310/AB1783-TriNKET® includes three polypeptides: scFv-AB1002-VL-VH-Fc (SEQ ID NO:198), A49MI-VH-CH1-Fc (SEQ ID NO:199), and A49MI-VL-CL (SEQ ID NO:200).

scFv-AB1002-VL-VH-Fc (″Chain S″)
(SEQ ID NO: 198)
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLVESGGGLVKPGGSLRLSCAASRFTFSDFGMHWVRQAPGKCLEWVSYISSGS
STIYYADSVKGRFTISRDNAKNSLYLQMNSLRAEDTAVYYCASSQSYYRGTLDYWGQGTTVT
VSSASDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG
A49MI-VH-CH1-Fc (″Chain H″)
(SEQ ID NO: 199)
EVQLVESGGGLVKPGGSLRLSCAASGFTFSSYSMNWVRQAPGKGLEWVSSISSSSSYIYYAD
SVKGRFTISRDNAKNSLYLQMNSLRAEDTAVYYCARGAPIGAAAGWFDPWGQGTLVTVSS
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVCTLPPSRDELTENQVSL
TCLVKGFYPSDIAVEWESNGQPENNYKTTPPVLDSDGSFFLYSWLTVDKSRWQQGNVFSCSV
MHEALHNHYTQKSLSLSPG
A49MI-VL-CL (″Chain L″)
(SEQ ID NO: 200)
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC

scFv-AB1002-VL-VH-Fc (SEQ ID NO:198) represents the full sequence of a 5T4 binding scFv linked to an Fc domain via a hinge comprising Ala-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:148, which includes a heavy chain variable domain of AB1002 connected to the C-terminus of a light chain variable domain of AB1002 via a (G₄S)₄ linker (SEQ ID NO:119). The scFv comprises substitution of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:198. In certain embodiments, the TriNKET® comprises an amino acid sequence at least 95%, identical to the amino acid sequence of SEQ ID NO:198. In certain embodiments, the TriNKET® comprises an amino acid sequence at least 96%, identical to the amino acid sequence of SEQ ID NO:198. In certain embodiments, the TriNKET® comprises an amino acid sequence at least 97%, identical to the amino acid sequence of SEQ ID NO:198. In certain embodiments, the TriNKET® comprises an amino acid sequence at least 98%, identical to the amino acid sequence of SEQ ID NO:198. In certain embodiments, the TriNKET® comprises an amino acid sequence at least 99%, identical to the amino acid sequence of SEQ ID NO:198. In certain embodiments, the TriNKET® comprises the amino acid sequence of SEQ ID NO:198.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-AB1002-VL-VH-Fc. In A49MI-VH-CH1-Fc, the Fc domain also includes K360E and K409W substitutions for heterodimerization with the Fc in scFv-AB1002-VL-VH-Fc.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:199. In certain embodiments, the TriNKET® comprises an amino acid sequence at least 95%, identical to the amino acid sequence of SEQ ID NO:199. In certain embodiments, the TriNKET® comprises an amino acid sequence at least 96%, identical to the amino acid sequence of SEQ ID NO:199. In certain embodiments, the TriNKET® comprises an amino acid sequence at least 97%, identical to the amino acid sequence of SEQ ID NO:199. In certain embodiments, the TriNKET® comprises an amino acid sequence at least 98%, identical to the amino acid sequence of SEQ ID NO:199. In certain embodiments, the TriNKET® comprises an amino acid sequence at least 99%, identical to the amino acid sequence of SEQ ID NO:199. In certain embodiments, the TriNKET® comprises the amino acid sequence of SEQ ID NO:199.

A49MI-VL-CL (SEQ ID NO:200) represents the light chain portion of the Fab fragment comprising a light chain variable domain of NKG2D-binding A49MI (SEQ ID NO:85) and a light chain constant domain.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:200. In certain embodiments, the TriNKET® comprises an amino acid sequence at least 95%, identical to the amino acid sequence of SEQ ID NO:200. In certain embodiments, the TriNKET® comprises an amino acid sequence at least 96%, identical to the amino acid sequence of SEQ ID NO:200. In certain embodiments, the TriNKET® comprises an amino acid sequence at least 97%, identical to the amino acid sequence of SEQ ID NO:200. In certain embodiments, the TriNKET® comprises an amino acid sequence at least 98%, identical to the amino acid sequence of SEQ ID NO:200. In certain embodiments, the TriNKET® comprises an amino acid sequence at least 99%, identical to the amino acid sequence of SEQ ID NO:200. In certain embodiments, the TriNKET® comprises the amino acid sequence of SEQ ID NO:200.

In certain embodiments, the Fc domain linked to the scFv includes K360E and K409W substitutions for heterodimerization and an Y349C substitution for forming a disulfide bond with a S354C substitution in A49MI-VH-CH1-Fc, which includes Q347R, D399V, and F405T.

In certain embodiments of the TriNKET® described above, portions of the chains within the TriNKET® form binding sites that bind a specific target protein. Accordingly, in some embodiments, an antigen binding site that binds to NKG2D (e.g., a human NKG2D-binding site) is formed by a VH in SEQ ID NO:199 (SEQ ID NO:95) and a VL in SEQ ID NO:200 (SEQ ID NO:85). In some embodiments, an antigen binding site that binds to 5T4 (e.g., a human 5T4-binding site) is formed by a VH in SEQ ID NO:198 (SEQ ID NO:146) and a VL in SEQ ID NO:198 (SEQ ID NO:147). In some embodiments, a binding site that binds CD16 (e.g., human CD16a-binding site) is formed by an Fc binding domain polypeptide in SEQ ID NO:198 and an Fc binding domain polypeptide in SEQ ID NO:199.

In certain embodiments of the TriNKET® described above, additional capabilities can be found. For example, in some embodiments, a disulfide bridge is formed between C44 in SEQ ID NO:146 and C100 in SEQ ID NO:147, numbered under the Kabat numbering scheme. In some embodiments, a disulfide bridge is formed between C349 in SEQ ID NO:199 and C354 in SEQ ID NO:198, numbered according to the EU numbering system. In some embodiments, a heterodimer is formed between an Fc domain in SEQ ID NO:198 and an Fc domain in SEQ ID NO:199. In some embodiments, the TriNKET® is capable of binding to NKG2D (e.g., human NKG2D) and CD16 (e.g., human CD16a) on the surface of an NK cell and to 5T4 (e.g., human 5T4) on the surface of a tumor cell.

Another TriNKET® described in the present disclosure is AB1310/AB1783-VH-VL-TriNKET®. AB1310/AB1783 VH-VL-TriNKET® includes (a) a 5T4-binding scFv sequence comprising the VH and VL sequences of AB1002 described in Table 2, in the orientation of VH positioned N-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1310/AB1783-VH-VL-TriNKET® includes three polypeptides: scFv-AB1002-VH-VL-Fc (SEQ ID NO:201), A49MI-VH-CH1-Fc (SEQ ID NO:199), and A49MI-VL-CL (SEQ ID NO:200).

scFv-AB1002-VH-VL-Fc (″Chain S″)
(SEQ ID NO: 201)
EVQLVESGGGLVKPGGSLRLSCAASRFTFSDFGMHWVRQAPGKCLEWVSYISSGSSTIYYAD
SVKGRFTISRDNAKNSLYLQMNSLRAEDTAVYYCASSQSYYRGTLDYWGQGTTVTVSS
GGGGSGGGGSGGGGSGGGGS
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIK
AS
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPRVYTLPPCRDELTKNQVSLTCLVKGFYPSDIAVEWESNGQPENNYKTTPPVLVSDGSFTL
YSKLTVDKSRWQQGNVFSCSVMHEALHNHYTQKSLSLSPG

scFv-AB1002-VH-VL-Fc (SEQ ID NO:201) represents the full sequence of a 5T4 binding scFv linked to an Fc domain via a hinge comprising Ala-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:149, which includes a heavy chain variable domain of AB1002 connected to the N-terminus of a light chain variable domain of AB1002 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:201. In certain embodiments, the TriNKET® comprises an amino acid sequence at least 95%, identical to the amino acid sequence of SEQ ID NO:201. In certain embodiments, the TriNKET® comprises an amino acid sequence at least 96%, identical to the amino acid sequence of SEQ ID NO:201. In certain embodiments, the TriNKET® comprises an amino acid sequence at least 97%, identical to the amino acid sequence of SEQ ID NO:201. In certain embodiments, the TriNKET® comprises an amino acid sequence at least 98%, identical to the amino acid sequence of SEQ ID NO:201. In certain embodiments, the TriNKET® comprises an amino acid sequence at least 99%, identical to the amino acid sequence of SEQ ID NO:201. In certain embodiments, the TriNKET® comprises the amino acid sequence of SEQ ID NO:201.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-AB1002-VH-VL-Fc. In A49MI-VH-CH1-Fc, the Fc domain also includes K360E and K409W substitutions for heterodimerization with the Fc in scFv-AB1002-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB2092-VL-VH-TriNKET®. AB2092-TriNKET® includes (a) a 5T4-binding scFv sequence comprising the VH and VL sequences of 05H04 described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB2092-VL-VH-TriNKET® includes three polypeptides: scFv-05H04-VL-VH-Fc (SEQ ID NO:220), A49MI-VH-CH1-Fc (SEQ ID NO:199), and A49MI-VL-CL (SEQ ID NO:200).

scFv-05H04-VL-VH-Fc (″Chain S″)
(SEQ ID NO: 220)
ENVLTQSPATLSLSPGERATLSCSAKSSVSYIHWYQQKPGQAPRLLIYDTSYLGSGIPARES
GSGSGTDYTLTISSLEPEDFAVYYCQQWSSYPYTFGCGTKVEIK
GGGGSGGGGSGGGGSGGGGS
QVQLVQSGAEVKKPGASVKVSCKASGYKFTDYYMDWVRQAPGQCLEWIGYIFPNDASTTYNE
KFKGKATLTADKSTNTAYMELSSLRSEDTAVYYCARSRDADYWGQGTTVTVSS
GS
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPRVYTLPPCRDELTKNQVSLTCLVKGFYPSDIAVEWESNGQPENNYKTTPPVLVSDGSFTL
YSKLTVDKSRWQQGNVFSCSVMHEALHNHYTQKSLSLSPG

scFv-05H04-VL-VH-Fc (SEQ ID NO:220) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:221, which includes a heavy chain variable domain of 05H04 connected to the C-terminus of a light chain variable domain of 05H04 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:220. In certain embodiments, the TriNKET® comprises an amino acid sequence at least 95%, identical to the amino acid sequence of SEQ ID NO:220. In certain embodiments, the TriNKET® comprises an amino acid sequence at least 96%, identical to the amino acid sequence of SEQ ID NO:220. In certain embodiments, the TriNKET® comprises an amino acid sequence at least 97%, identical to the amino acid sequence of SEQ ID NO:220. In certain embodiments, the TriNKET® comprises an amino acid sequence at least 98%, identical to the amino acid sequence of SEQ ID NO:200. In certain embodiments, the TriNKET® comprises an amino acid sequence at least 99%, identical to the amino acid sequence of SEQ ID NO:220. In certain embodiments, the TriNKET® comprises the amino acid sequence of SEQ ID NO:220.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-05H04-VL-VH-Fc. In A49MI-VH-CH1-Fc, the Fc domain also includes K360E and K409W substitutions for heterodimerization with the Fc in scFv-05H04-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB2093-VH-VL-TriNKET®. AB2093-TriNKET® includes (a) a 5T4-binding scFv sequence comprising the VH and VL sequences of 05H04 described in Table 2, in the orientation of VH positioned N-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB2093-VH-VL-TriNKET® includes three polypeptides: scFv-05H04-VH-VL-Fc (SEQ ID NO:222), A49MI-VH-CH1-Fc (SEQ ID NO:199), and A49MI-VL-CL (SEQ ID NO:200).

scFv-05H04-VH-VL-Fc (″Chain S″)
(SEQ ID NO: 222)
QVQLVQSGAEVKKPGASVKVSCKASGYKFTDYYMDWVRQAPGQCLEWIGYIFPNDASTTYNE
KFKGKATLTADKSTNTAYMELSSLRSEDTAVYYCARSRDADYWGQGTTVTVSS
GGGGSGGGGSGGGGSGGGGS
ENVLTQSPATLSLSPGERATLSCSAKSSVSYIHWYQQKPGQAPRLLIYDTSYLGSGIPARES
GSGSGTDYTLTISSLEPEDFAVYYCQQWSSYPYTFGCGTKVEIK
GS
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPRVYTLPPCRDELTKNQVSLTCLVKGFYPSDIAVEWESNGQPENNYKTTPPVLVSDGSFTL
YSKLTVDKSRWQQGNVFSCSVMHEALHNHYTQKSLSLSPG

scFv-05H04-VH-VL-Fc (SEQ ID NO:222) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:159, which includes a heavy chain variable domain of 05H04 connected to the N-terminus of a light chain variable domain of 05H04 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:222. In certain embodiments, the TriNKET® comprises an amino acid sequence at least 95%, identical to the amino acid sequence of SEQ ID NO:222. In certain embodiments, the TriNKET® comprises an amino acid sequence at least 96%, identical to the amino acid sequence of SEQ ID NO:222. In certain embodiments, the TriNKET® comprises an amino acid sequence at least 97%, identical to the amino acid sequence of SEQ ID NO:222. In certain embodiments, the TriNKET® comprises an amino acid sequence at least 98%, identical to the amino acid sequence of SEQ ID NO:222. In certain embodiments, the TriNKET® comprises an amino acid sequence at least 99%, identical to the amino acid sequence of SEQ ID NO:222. In certain embodiments, the TriNKET® comprises the amino acid sequence of SEQ ID NO:222.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-05H04-VH-VL-Fc. In A49MI-VH-CH1-Fc, the Fc domain also includes K360E and K409W substitutions for heterodimerization with the Fc in scFv-05H04-VH-VL-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB2143-VH-VL-Q1E-TriNKET®. AB2143-Q1E-TriNKET® includes (a) a 5T4-binding scFv sequence comprising the VH and VL sequences of 05H04-Q1E described in Table 2, in the orientation of VH positioned N-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB2143-VH-VL-TriNKET® includes three polypeptides: scFv-05H04-VH-VL-Q1E-Fc (SEQ ID NO:223), A49MI-VH-CH1-Fc (SEQ ID NO:199), and A49MI-VL-CL (SEQ ID NO:200).

scFv-05H04-VH-VL-Q1E-Fc (″Chain S″)
(SEQ ID NO: 223)
EVQLVQSGAEVKKPGASVKVSCKASGYKFTDYYMDWVRQAPGQCLEWIGYIFPNDASTTYNE
KFKGKATLTADKSTNTAYMELSSLRSEDTAVYYCARSRDADYWGQGTTVTVSS
GGGGSGGGGSGGGGSGGGGS
ENVLTQSPATLSLSPGERATLSCSAKSSVSYIHWYQQKPGQAPRLLIYDTSYLGSGIPARES
GSGSGTDYTLTISSLEPEDFAVYYCQQWSSYPYTFGCGTKVEIK
GS
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPRVYTLPPCRDELTKNQVSLTCLVKGFYPSDIAVEWESNGQPENNYKTTPPVLVSDGSFTL
YSKLTVDKSRWQQGNVFSCSVMHEALHNHYTQKSLSLSPG

scFv-05H04-VH-VL-Q1E-Fc (SEQ ID NO:223) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:160, which includes a heavy chain variable domain of 05H04-Q1E connected to the N-terminus of a light chain variable domain of 05H04 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:223. In certain embodiments, the TriNKET® comprises an amino acid sequence at least 95%, identical to the amino acid sequence of SEQ ID NO:223. In certain embodiments, the TriNKET® comprises an amino acid sequence at least 96%, identical to the amino acid sequence of SEQ ID NO:223. In certain embodiments, the TriNKET® comprises an amino acid sequence at least 97%, identical to the amino acid sequence of SEQ ID NO:223. In certain embodiments, the TriNKET® comprises an amino acid sequence at least 98%, identical to the amino acid sequence of SEQ ID NO:223. In certain embodiments, the TriNKET® comprises an amino acid sequence at least 99%, identical to the amino acid sequence of SEQ ID NO:223. In certain embodiments, the TriNKET® comprises the amino acid sequence of SEQ ID NO:223.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-05H04-Q1E-VH-VL-Fc. In A49MI-VH-CH1-Fc, the Fc domain also includes K360E and K409W substitutions for heterodimerization with the Fc in scFv-05H04-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1878-VL-VH TriNKET®. AB1878-TriNKET® includes (a) a 5T4-binding scFv sequence comprising the VH and VL sequences of 10F10 21*05 described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1878-TriNKET® includes three polypeptides: scFv-10F10 21*05-VL-VH-Fc (SEQ ID NO:224), A49MI-VH-CH1-Fc (SEQ ID NO:199), and A49MI-VL-CL (SEQ ID NO:200).

scFv-10F10 21*05-VL-VH-Fc (″Chain S″)
(SEQ ID NO: 224)
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIK
GGGGSGGGGSGGGGSGGGGS
EVQLVESGGGLVKPGGSLRLSCAASRFTFSDFGMHWVRQAPGKCLEWVSYISSGSSTIYYAD
TVKGRFTISRDNAKNSLYLQMNSLRAEDTAVYYCASSQSYYRGTLDYWGQGTTVTVSS
GS
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPRVYTLPPCRDELTKNQVSLTCLVKGFYPSDIAVEWESNGQPENNYKTTPPVLVSDGSFTL
YSKLTVDKSRWQQGNVFSCSVMHEALHNHYTQKSLSLSPG

scFv-10F10 21*05-VL-VH-Fc (SEQ ID NO:224) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:167, which includes a heavy chain variable domain of 10F10 21*05 connected to the C-terminus of a light chain variable domain of 10F10 21*05 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:224. In certain embodiments, the TriNKET® comprises an amino acid sequence at least 95%, identical to the amino acid sequence of SEQ ID NO:224. In certain embodiments, the TriNKET® comprises an amino acid sequence at least 96%, identical to the amino acid sequence of SEQ ID NO:224. In certain embodiments, the TriNKET® comprises an amino acid sequence at least 97%, identical to the amino acid sequence of SEQ ID NO:224. In certain embodiments, the TriNKET® comprises an amino acid sequence at least 98%, identical to the amino acid sequence of SEQ ID NO:224. In certain embodiments, the TriNKET® comprises an amino acid sequence at least 99%, identical to the amino acid sequence of SEQ ID NO:224. In certain embodiments, the TriNKET® comprises the amino acid sequence of SEQ ID NO:224.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-10F10 21*05-VL-VH-Fc. In A49MI-VH-CH1-Fc, the Fc domain also includes K360E and K409W substitutions for heterodimerization with the Fc in scFv-10F10 21*05-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1881-VL-VH TriNKET®. AB1881-TriNKET® includes (a) a 5T4-binding scFv sequence comprising the VH and VL sequences of 10F10 23*03 described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1881-TriNKET® includes three polypeptides: scFv-10F10 23*03-VL-VH-Fc (SEQ ID NO:225), A49MI-VH-CH1-Fc (SEQ ID NO:199), and A49MI-VL-CL (SEQ ID NO:200).

scFv-10F10 21*03-VL-VH-Fc (″Chain S″)
(SEQ ID NO: 225)
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIK
GGGGSGGGGSGGGGSGGGGS
EVQLLESGGGLVQPGGSLRLSCAASRFTFSDFGMHWVRQAPGKCLEWIAYISSGSSTIYYAD
TVKGRFTISRDNAKNTLYLQMNSLRAEDTAVYYCASSQSYYRGTLDYWGQGTTVTVSS
GS
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPRVYTLPPCRDELTKNQVSLTCLVKGFYPSDIAVEWESNGQPENNYKTTPPVLVSDGSFTL
YSKLTVDKSRWQQGNVFSCSVMHEALHNHYTQKSLSLSPG

scFv-10F10 23*03-VL-VH-Fc (SEQ ID NO:225) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:169, which includes a heavy chain variable domain of 10F10 23*03 connected to the C-terminus of a light chain variable domain of 10F10 23*03 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:225. In certain embodiments, the TriNKET® comprises an amino acid sequence at least 95%, identical to the amino acid sequence of SEQ ID NO:225. In certain embodiments, the TriNKET® comprises an amino acid sequence at least 96%, identical to the amino acid sequence of SEQ ID NO:225. In certain embodiments, the TriNKET® comprises an amino acid sequence at least 97%, identical to the amino acid sequence of SEQ ID NO:225. In certain embodiments, the TriNKET® comprises an amino acid sequence at least 98%, identical to the amino acid sequence of SEQ ID NO:225. In certain embodiments, the TriNKET® comprises an amino acid sequence at least 99%, identical to the amino acid sequence of SEQ ID NO:225. In certain embodiments, the TriNKET® comprises the amino acid sequence of SEQ ID NO:225.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-10F10 23*03-VL-VH-Fc. In A49MI-VH-CH1-Fc, the Fc domain also includes K360E and K409W substitutions for heterodimerization with the Fc in scFv-10F10 23*03-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1882-VL-VH TriNKET®. AB1882-TriNKET® includes (a) a 5T4-binding scFv sequence comprising the VH and VL sequences of 10F10 48*01 described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1882-TriNKET® includes three polypeptides: scFv-10F10 48*01-VL-VH-Fc (SEQ ID NO:226), A49MI-VH-CH1-Fc (SEQ ID NO:199), and A49MI-VL-CL (SEQ ID NO:200).

scFv-10F10 48*01-VL-VH-Fc (″Chain S″)
(SEQ ID NO: 226)
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIK
GGGGSGGGGSGGGGSGGGGS
EVQLVESGGGLVQPGGSLRLSCAASRFTFSDFGMHWVRQAPGKCLEWVSYISSGSSTIYYAD
TVKGRFTISRDNAKNSLYLQMNSLRAEDTAVYYCASSQSYYRGTLDYWGQGTTVTVSS
GS
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPRVYTLPPCRDELTKNQVSLTCLVKGFYPSDIAVEWESNGQPENNYKTTPPVLVSDGSFTL
YSKLTVDKSRWQQGNVFSCSVMHEALHNHYTQKSLSLSPG

scFv-10F10 48*01-VL-VH-Fc (SEQ ID NO:226) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:171, which includes a heavy chain variable domain of 10F10 48*01 connected to the C-terminus of a light chain variable domain of 10F10 48*01 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:226. In certain embodiments, the TriNKET® comprises an amino acid sequence at least 95%, identical to the amino acid sequence of SEQ ID NO:226. In certain embodiments, the TriNKET® comprises an amino acid sequence at least 96%, identical to the amino acid sequence of SEQ ID NO:226. In certain embodiments, the TriNKET® comprises an amino acid sequence at least 97%, identical to the amino acid sequence of SEQ ID NO:226. In certain embodiments, the TriNKET® comprises an amino acid sequence at least 98%, identical to the amino acid sequence of SEQ ID NO:226. In certain embodiments, the TriNKET® comprises an amino acid sequence at least 99%, identical to the amino acid sequence of SEQ ID NO:226. In certain embodiments, the TriNKET® comprises the amino acid sequence of SEQ ID NO:226.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-10F10 48*01-VL-VH-Fc. In A49MI-VH-CH1-Fc, the Fc domain also includes K360E and K409W substitutions for heterodimerization with the Fc in scFv-10F10 48*01-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1884-VL-VH TriNKET®. AB1884-TriNKET® includes (a) a 5T4-binding scFv sequence comprising the VH and VL sequences of 10F10 48*01 BM2 scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1884-TriNKET® includes three polypeptides: scFv-10F10 48*01 BM2-VL-VH-Fc (SEQ ID NO:227), A49MI-VH-CH1-Fc (SEQ ID NO:199), and A49MI-VL-CL (SEQ ID NO:200).

scFv-10F10 48*01 BM2-VL-VH-Fc (″Chain S″)
(SEQ ID NO: 227)
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIK
GGGGSGGGGSGGGGSGGGGS
EVQLVESGGGLVQPGGSLRLSCAASRFTFSDFGMHWVRQAPGKCLEWIAYISSGSSTIYYAD
TVKGRFTISRDNAKNSLYLQMNSLRAEDTAVYYCASSQSYYRGTLDYWGQGTTVTVSS
GS
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGOPR
EPRVYTLPPCRDELTKNQVSLTCLVKGFYPSDIAVEWESNGQPENNYKTTPPVLVSDGSFTL
YSKLTVDKSRWQQGNVFSCSVMHEALHNHYTQKSLSLSPG

scFv-10F10 48*01 BM2-VL-VH-Fc (SEQ ID NO:227) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:229, which includes a heavy chain variable domain of 10F10 48*01 BM2 connected to the C-terminus of a light chain variable domain of 10F10 48*01 BM2 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:227. In certain embodiments, the TriNKET® comprises an amino acid sequence at least 95%, identical to the amino acid sequence of SEQ ID NO:227. In certain embodiments, the TriNKET® comprises an amino acid sequence at least 96%, identical to the amino acid sequence of SEQ ID NO:227. In certain embodiments, the TriNKET® comprises an amino acid sequence at least 97%, identical to the amino acid sequence of SEQ ID NO:227. In certain embodiments, the TriNKET® comprises an amino acid sequence at least 98%, identical to the amino acid sequence of SEQ ID NO:227. In certain embodiments, the TriNKET® comprises an amino acid sequence at least 99%, identical to the amino acid sequence of SEQ ID NO:227. In certain embodiments, the TriNKET® comprises the amino acid sequence of SEQ ID NO:227.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-10F10 48*01 BM2-VL-VH-Fc. In A49MI-VH-CH1-Fc, the Fc domain also includes K360E and K409W substitutions for heterodimerization with the Fc in scFv-10F10 48*01 BM3-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1885-VL-VH TriNKET®. AB1885-TriNKET® includes (a) a 5T4-binding scFv sequence comprising the VH and VL sequences of 10F10 11*01 scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1885-TriNKET® includes three polypeptides: scFv-10F10 11*01-VL-VH-Fc (SEQ ID NO:230), A49MI-VH-CH1-Fc (SEQ ID NO:199), and A49MI-VL-CL (SEQ ID NO:200).

scFv-10F10 11*01-VL-VH-Fc (″Chain S″)
(SEQ ID NO: 230)
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIK
GGGGSGGGGSGGGGSGGGGS
QVQLVESGGGLVKPGGSLRLSCAASRFTFSDFGMHWVRQAPGKCLEWIAYISSGSSTIYYAD
TVKGRFTISRDNAKNSLYLQMNSLRAEDTAVYYCASSQSYYRGTLDYWGQGTTVTVSS
GS
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPRVYTLPPCRDELTKNQVSLTCLVKGFYPSDIAVEWESNGQPENNYKTTPPVLVSDGSFTL
YSKLTVDKSRWQQGNVFSCSVMHEALHNHYTQKSLSLSPG

scFv-10F10 11*01-VL-VH-Fc (SEQ ID NO:230) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:173, which includes a heavy chain variable domain of 10F10 11*01 connected to the C-terminus of a light chain variable domain of 10F10 11*01 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:230. In certain embodiments, the TriNKET® comprises an amino acid sequence at least 95%, identical to the amino acid sequence of SEQ ID NO:230. In certain embodiments, the TriNKET® comprises an amino acid sequence at least 96%, identical to the amino acid sequence of SEQ ID NO:230. In certain embodiments, the TriNKET® comprises an amino acid sequence at least 97%, identical to the amino acid sequence of SEQ ID NO:230. In certain embodiments, the TriNKET® comprises an amino acid sequence at least 98%, identical to the amino acid sequence of SEQ ID NO:230. In certain embodiments, the TriNKET® comprises an amino acid sequence at least 99%, identical to the amino acid sequence of SEQ ID NO:230. In certain embodiments, the TriNKET® comprises the amino acid sequence of SEQ ID NO:230.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-10F10 11*01-VL-VH-Fc. In A49MI-VH-CH1-Fc, the Fc domain also includes K360E and K409W substitutions for heterodimerization with the Fc in scFv-10F10 11*01-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1886-VL-VH TriNKET®. AB1886-TriNKET® includes (a) a 5T4-binding scFv sequence comprising the VH and VL sequences of 10F10-11*01 BM1 scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1886-TriNKET® includes three polypeptides: scFv-10F10-11*01 BM1-VL-VH-Fc (SEQ ID NO:231), A49MI-VH-CH1-Fc (SEQ ID NO:199), and A49MI-VL-CL (SEQ ID NO:200).

scFv-10F10 11*01-VL-VH-Fc (″Chain S″)
(SEQ ID NO: 231)
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIK
GGGGSGGGGSGGGGSGGGGS
EVQLVESGGGLVKPGGSLRLSCAASRFTFSDFGMHWIRQAPGKCLEWVSYISSGSSTIYYAD
TVKGRFTISRDNAKNSLYLQMNSLRAEDTAVYYCASSQSYYRGTLDYWGQGTTVTVSS
GS
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPRVYTLPPCRDELTKNQVSLTCLVKGFYPSDIAVEWESNGQPENNYKTTPPVLVSDGSFTL
YSKLTVDKSRWQQGNVFSCSVMHEALHNHYTQKSLSLSPG

scFv-10F10 11*01 BM1-VL-VH-Fc (SEQ ID NO:231) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:233, which includes a heavy chain variable domain of 10F10 11*01 BM1 connected to the C-terminus of a light chain variable domain of 10F10 11*01 BM1 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:231. In certain embodiments, the TriNKET® comprises an amino acid sequence at least 95%, identical to the amino acid sequence of SEQ ID NO:231. In certain embodiments, the TriNKET® comprises an amino acid sequence at least 96%, identical to the amino acid sequence of SEQ ID NO:231. In certain embodiments, the TriNKET® comprises an amino acid sequence at least 97%, identical to the amino acid sequence of SEQ ID NO:231. In certain embodiments, the TriNKET® comprises an amino acid sequence at least 98%, identical to the amino acid sequence of SEQ ID NO:231. In certain embodiments, the TriNKET® comprises an amino acid sequence at least 99%, identical to the amino acid sequence of SEQ ID NO:231. In certain embodiments, the TriNKET® comprises the amino acid sequence of SEQ ID NO:231.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-10F10 11*01 BM1-VL-VH-Fc. In A49MI-VH-CH1-Fc, the Fc domain also includes K360E and K409W substitutions for heterodimerization with the Fc in scFv-10F10 11*01 BM1-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1887-VL-VH TriNKET®. AB1887-TriNKET® includes (a) a 5T4-binding scFv sequence comprising the VH and VL sequences of 10F10-21*05-scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1887-TriNKET® includes three polypeptides: scFv-10F10 21*05-VL-VH-Fc (SEQ ID NO:234), A49MI-VH-CH1-Fc (SEQ ID NO:199), and A49MI-VL-CL (SEQ ID NO:200).

scFv-10F10 21*05-VL-VH-Fc (″Chain S″)
(SEQ ID NO: 234)
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIK
GGGGSGGGGSGGGGSGGGGS
EVQLVESGGGLVKPGGSLRLSCAASRFTFSDFGMHWVRQAPGKCLEWIAYISSGSSTIYYAD
TVKGRFTISRDNAKNSLYLQMNSLRAEDTAVYYCASSQSYYRGTLDYWGQGTTVTVSS
GS
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPRVYTLPPCRDELTKNQVSLTCLVKGFYPSDIAVEWESNGQPENNYKTTPPVLVSDGSFTL
YSKLTVDKSRWQQGNVFSCSVMHEALHNHYTQKSLSLSPG

scFv-10F10-21*05-VL-VH-Fc (SEQ ID NO:234) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:175, which includes a heavy chain variable domain of 10F10-21*05 connected to the C-terminus of a light chain variable domain of 10F10-21*05 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:234. In certain embodiments, the TriNKET® comprises an amino acid sequence at least 95%, identical to the amino acid sequence of SEQ ID NO:234. In certain embodiments, the TriNKET® comprises an amino acid sequence at least 96%, identical to the amino acid sequence of SEQ ID NO:234. In certain embodiments, the TriNKET® comprises an amino acid sequence at least 97%, identical to the amino acid sequence of SEQ ID NO:234. In certain embodiments, the TriNKET® comprises an amino acid sequence at least 98%, identical to the amino acid sequence of SEQ ID NO:234. In certain embodiments, the TriNKET® comprises an amino acid sequence at least 99%, identical to the amino acid sequence of SEQ ID NO:234. In certain embodiments, the TriNKET® comprises the amino acid sequence of SEQ ID NO:234.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-10F10-21*05-VL-VH-Fc. In A49MI-VH-CH1-Fc, the Fc domain also includes K360E and K409W substitutions for heterodimerization with the Fc in scFv-10F10-21*05-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1892-VL-VH TriNKET®. AB1892-TriNKET® includes (a) a 5T4-binding scFv sequence comprising the VH and VL sequences of the 10F10-23*03 BM1 scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1892-TriNKET® includes three polypeptides: scFv-10F10-23*03 BM1-VL-VH-Fc (SEQ ID NO:235), A49MI-VH-CH1-Fc (SEQ ID NO:199), and A49MI-VL-CL (SEQ ID NO:200).

scFv-10F10-23*03 BM1-VL-VH-Fc (″Chain S″)
(SEQ ID NO: 235)
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIK
GGGGSGGGGSGGGGSGGGGS
EVQLLESGGGLVQPGGSLRLSCAASRFTFSDFGMHWVRQAPGKCLEWVSYISSGSSTIYYAD
TVKGRFTISRDNSKNTLYLQMNSLRAEDTAVYYCASSQSYYRGTLDYWGQGTTVTVSS
GS
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPRVYTLPPCRDELTKNQVSLTCLVKGFYPSDIAVEWESNGQPENNYKTTPPVLVSDGSFTL
YSKLTVDKSRWQQGNVFSCSVMHEALHNHYTQKSLSLSPG

scFv-10F10-23*03 BM1-VL-VH-Fc (SEQ ID NO:235) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:293, which includes a heavy chain variable domain of 10F10-23*03 BM1 connected to the C-terminus of a light chain variable domain of 10F10-23*03 BM1 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:235. In certain embodiments, the TriNKET® comprises an amino acid sequence at least 95%, identical to the amino acid sequence of SEQ ID NO:235. In certain embodiments, the TriNKET® comprises an amino acid sequence at least 96%, identical to the amino acid sequence of SEQ ID NO:235. In certain embodiments, the TriNKET® comprises an amino acid sequence at least 97%, identical to the amino acid sequence of SEQ ID NO:235. In certain embodiments, the TriNKET® comprises an amino acid sequence at least 98%, identical to the amino acid sequence of SEQ ID NO:235. In certain embodiments, the TriNKET® comprises an amino acid sequence at least 99%, identical to the amino acid sequence of SEQ ID NO:235. In certain embodiments, the TriNKET® comprises the amino acid sequence of SEQ ID NO:235.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-10F10-23*03 BM1-VL-VH-Fc. In A49MI-VH-CH1-Fc, the Fc domain also includes K360E and K409W substitutions for heterodimerization with the Fc in scFv-10F10-23*03 BM1-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1319-VL-VH TriNKET®. AB1319-TriNKET® includes (a) a 5T4-binding scFv sequence comprising the VH and VL sequences of the 11F09 21*05 scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1319-TriNKET® includes three polypeptides: scFv-11F09 21*05-VL-VH-Fc (SEQ ID NO:237), A49MI-VH-CH1-Fc (SEQ ID NO:199), and A49MI-VL-CL (SEQ ID NO:200).

scFv-11F09 21*05-VL-VH-Fc (“Chain S”)
(SEQ ID NO: 237)
DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVKPGGSLR
LSCEASGFTFSDYGMHWVRQAPGKCLEWLAYISSGSSTFYYADTVKGRFT
ISRDNAKNSLYLQMNSLRAEDTAVYYCGSSQSYYRGTMDYWGQGTTVTVS
SASDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGOPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG

scFv-11F09 21*05-VL-VH-Fc (SEQ ID NO:237) represents the full sequence of a 5T4 binding scFv linked to an Fc domain via a hinge comprising Ala-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:202, which includes a heavy chain variable domain of 11F09 21*05 connected to the C-terminus of a light chain variable domain of 11F09 21*05 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:237. In certain embodiments, the TriNKET® comprises an amino acid sequence at least 95%, identical to the amino acid sequence of SEQ ID NO:237. In certain embodiments, the TriNKET® comprises an amino acid sequence at least 96%, identical to the amino acid sequence of SEQ ID NO:237. In certain embodiments, the TriNKET® comprises an amino acid sequence at least 97%, identical to the amino acid sequence of SEQ ID NO:237. In certain embodiments, the TriNKET® comprises an amino acid sequence at least 98%, identical to the amino acid sequence of SEQ ID NO:237. In certain embodiments, the TriNKET® comprises an amino acid sequence at least 99%, identical to the amino acid sequence of SEQ ID NO:237. In certain embodiments, the TriNKET® comprises the amino acid sequence of SEQ ID NO:237.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-11F09 21*05-VL-VH-Fc. In A49MI-VH-CH1-Fc, the Fc domain also includes K360E and K409W substitutions for heterodimerization with the Fc in scFv-11F09 21*05-VL-VH-Fc.

A49M1-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1879-VL-VH TriNKET®. AB1879-TriNKET® includes (a) a 5T4-binding scFv sequence comprising the VH and VL sequences of the 11F09 48*01 scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1879-TriNKET® includes three polypeptides: scFv-11F09 48*01-VL-VH-Fc (SEQ ID NO:238), A49MI-VH-CH1-Fc (SEQ ID NO:199), and A49MI-VL-CL (SEQ ID NO:200).

scFv-11F09 48*01-VL-VH-Fc (“Chain S”)
(SEQ ID NO: 238)
DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVQPGGSLR
LSCAASGFTFSDYGMHWVRQAPGKCLEWVSYISSGSSTFYYADTVKGRFT
ISRDNAKNSLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG

scFv-11F09 48*01-VL-VH-Fc (SEQ ID NO:238) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:179, which includes a heavy chain variable domain of 11F09 48*01 connected to the C-terminus of a light chain variable domain of 11F09 48*01 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:238. In certain embodiments, the TriNKET® comprises an amino acid sequence at least 95%, identical to the amino acid sequence of SEQ ID NO:238. In certain embodiments, the TriNKET® comprises an amino acid sequence at least 96%, identical to the amino acid sequence of SEQ ID NO:238. In certain embodiments, the TriNKET® comprises an amino acid sequence at least 97%, identical to the amino acid sequence of SEQ ID NO:238. In certain embodiments, the TriNKET® comprises an amino acid sequence at least 98%, identical to the amino acid sequence of SEQ ID NO:238. In certain embodiments, the TriNKET® comprises an amino acid sequence at least 99%, identical to the amino acid sequence of SEQ ID NO:238. In certain embodiments, the TriNKET® comprises the amino acid sequence of SEQ ID NO:238.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-11F09 48*01-VL-VH-Fc. In A49MI-VH-CH1-Fc, the Fc domain also includes K360E and K409W substitutions for heterodimerization with the Fc in scFv-11F09 48*01-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1880-VL-VH TriNKET®. AB1880-TriNKET® includes (a) a 5T4-binding scFv sequence comprising the VH and VL sequences of the 11F09 21*05 scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1880-TriNKET® includes three polypeptides: scFv-11F09 21*05-VL-VH-Fc (SEQ ID NO:239), A49MI-VH-CH1-Fc (SEQ ID NO:199), and A49MI-VL-CL (SEQ ID NO:200).

scFv-11F09 21*05-VL-VH-Fc (“Chain S”)
(SEQ ID NO: 239)
DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVKPGGSLR
LSCEASGFTFSDYGMHWVRQAPGKCLEWLAYISSGSSTFYYADTVKGRFT
ISRDNAKNSLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG

scFv-11F09 21*05-VL-VH-Fc (SEQ ID NO:239) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:202, which includes a heavy chain variable domain of 21*05 connected to the C-terminus of a light chain variable domain of 21*05 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:239. In certain embodiments, the TriNKET® comprises an amino acid sequence at least 95%, identical to the amino acid sequence of SEQ ID NO:239. In certain embodiments, the TriNKET® comprises an amino acid sequence at least 96%, identical to the amino acid sequence of SEQ ID NO:239. In certain embodiments, the TriNKET® comprises an amino acid sequence at least 97%, identical to the amino acid sequence of SEQ ID NO:239. In certain embodiments, the TriNKET® comprises an amino acid sequence at least 98%, identical to the amino acid sequence of SEQ ID NO:239. In certain embodiments, the TriNKET® comprises an amino acid sequence at least 99%, identical to the amino acid sequence of SEQ ID NO:239. In certain embodiments, the TriNKET® comprises the amino acid sequence of SEQ ID NO:239.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-11F09 21*05-VL-VH-Fc. In A49MI-VH-CH1-Fc, the Fc domain also includes K360E and K409W substitutions for heterodimerization with the Fc in scFv-11F09 21*05-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1883-VL-VH TriNKET®. AB1883-TriNKET® includes (a) a 5T4-binding scFv sequence comprising the VH and VL sequences of 11F09 11*01 scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1883-TriNKET® includes three polypeptides: scFv-11F09 11*01-VL-VH-Fc (SEQ ID NO:240), A49MI-VH-CH1-Fc (SEQ ID NO:199), and A49MI-VL-CL (SEQ ID NO:200).

scFv-11F09 11*01-VL-VH-Fc (“Chain S”)
(SEQ ID NO: 240)
DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSQVQLVESGGGLVKPGGSLR
LSCAASGFTFSDYGMHWVRQAPGKCLEWLAYISSGSSTFYYADTVKGRFT
ISRDNAKNSLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG

scFv-11F09 11*01-VL-VH-Fc (SEQ ID NO:240) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:181, which includes a heavy chain variable domain of 11*01 connected to the C-terminus of a light chain variable domain of 11*01 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:240. In certain embodiments, the TriNKET® comprises an amino acid sequence at least 95%, identical to the amino acid sequence of SEQ ID NO:240. In certain embodiments, the TriNKET® comprises an amino acid sequence at least 96%, identical to the amino acid sequence of SEQ ID NO:240. In certain embodiments, the TriNKET® comprises an amino acid sequence at least 97%, identical to the amino acid sequence of SEQ ID NO:240. In certain embodiments, the TriNKET® comprises an amino acid sequence at least 98%, identical to the amino acid sequence of SEQ ID NO:240. In certain embodiments, the TriNKET® comprises an amino acid sequence at least 99%, identical to the amino acid sequence of SEQ ID NO:240. In certain embodiments, the TriNKET® comprises the amino acid sequence of SEQ ID NO:240.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-11F09 11*01-VL-VH-Fc. In A49MI-VH-CH1-Fc, the Fc domain also includes K360E and K409W substitutions for heterodimerization with the Fc in scFv-11F09 11*01-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1888-VL-VH TriNKET®. AB1888-TriNKET® includes (a) a 5T4-binding scFv sequence comprising the VH and VL sequences of the 11F09 21*05 BM1 scFv/AB1011 described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1888-TriNKET® includes three polypeptides: scFv-11F09 21*05 BM1-VL-VH-Fc (SEQ ID NO:241), A49MI-VH-CH1-Fc (SEQ ID NO:199), and A49MI-VL-CL (SEQ ID NO:200).

scFv-11F09 21*05 BM1-VL-VH-Fc (“Chain S”)
(SEQ ID NO: 241)
DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVKPGGSLR
LSCAASGFTFSDYGMHWVRQAPGKCLEWVSYISSGSSTFYYADTVKGRFT
ISRDNAKNSLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG

scFv-11F09 21*05 BM1-VL-VH-Fc (SEQ ID NO:241) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:271, which includes a heavy chain variable domain of 11F09 21*05 BM1 (AB1011 in Table 2) connected to the C-terminus of a light chain variable domain of 11F09 21*05 BM1 (AB1011 in Table 2) via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:241. In certain embodiments, the TriNKET® comprises an amino acid sequence at least 95%, identical to the amino acid sequence of SEQ ID NO:241. In certain embodiments, the TriNKET® comprises an amino acid sequence at least 96%, identical to the amino acid sequence of SEQ ID NO:241. In certain embodiments, the TriNKET® comprises an amino acid sequence at least 97%, identical to the amino acid sequence of SEQ ID NO:241. In certain embodiments, the TriNKET® comprises an amino acid sequence at least 98%, identical to the amino acid sequence of SEQ ID NO:241. In certain embodiments, the TriNKET® comprises an amino acid sequence at least 99%, identical to the amino acid sequence of SEQ ID NO:241. In certain embodiments, the TriNKET® comprises the amino acid sequence of SEQ ID NO:241.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-11F09 21*05 BM1-VL-VH-Fc. In A49MI-VH-CH1-Fc, the Fc domain also includes K360E and K409W substitutions for heterodimerization with the Fc in scFv-11F09 21*05 BM1-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1889-VL-VH TriNKET®. AB1889-TriNKET® includes (a) a 5T4-binding scFv sequence comprising the VH and VL sequences of the 11F09 48*01 BM2 scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1889-TriNKET® includes three polypeptides: scFv-11F09 48*01 BM2-VL-VH-Fc (SEQ ID NO:244), A49MI-VH-CH1-Fc (SEQ ID NO:199), and A49MI-VL-CL (SEQ ID NO:200).

scFv-11F09 48*01 BM2-VL-VH-Fc (“Chain S”)
(SEQ ID NO: 244)
DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVQPGGSLR
LSCAASGFTFSDYGMHWVRQAPGKCLEWLAYISSGSSTFYYADTVKGRFT
ISRDNAKNSLYLQMNSLRAEDTAVYYCGSSQSYYRGTMDYWGQGTTVTVS
SGSDKTHTCPPCPAPELLGGPSVELEPPKPKDTLMISRTPEVTCVVVDVS
HEDPEVKENWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG

scFv-11F09 48*01 BM2-VL-VH-Fc (SEQ ID NO:244) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:246, which includes a heavy chain variable domain of 11F09 48*01 BM2 connected to the C-terminus of a light chain variable domain of 11F09 48*01 BM2 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:244. In certain embodiments, the TriNKET® comprises an amino acid sequence at least 95%, identical to the amino acid sequence of SEQ ID NO:244. In certain embodiments, the TriNKET® comprises an amino acid sequence at least 96%, identical to the amino acid sequence of SEQ ID NO:244. In certain embodiments, the TriNKET® comprises an amino acid sequence at least 97%, identical to the amino acid sequence of SEQ ID NO:244. In certain embodiments, the TriNKET® comprises an amino acid sequence at least 98%, identical to the amino acid sequence of SEQ ID NO:244. In certain embodiments, the TriNKET® comprises an amino acid sequence at least 99%, identical to the amino acid sequence of SEQ ID NO:244. In certain embodiments, the TriNKET® comprises the amino acid sequence of SEQ ID NO:244.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-11F09 48*01 BM2-VL-VH-Fc. In A49MI-VH-CH1-Fc, the Fc domain also includes K360E and K409W substitutions for heterodimerization with the Fc in scFv-11F09 48*01 BM2-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1890-VL-VH TriNKET®. AB1890-TriNKET® includes (a) a 5T4-binding scFv sequence comprising the VH and VL sequences of the 11F09 23*03 scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1890-TriNKET® includes three polypeptides: scFv-11F09 23*03-VL-VH-Fc (SEQ ID NO:286), A49MI-VH-CH1-Fc (SEQ ID NO:199), and A49MI-VL-CL (SEQ ID NO:200).

scFv-11F09 23*03-VL-VH-Fc (“Chain S”)
(SEQ ID NO: 286)
DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLLESGGGLVQPGGSLR
LSCAASGFTFSDYGMHWVRQAPGKCLEWVSYISSGSSTFYYADTVKGRFT
ISRDNSKNTLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG

scFv-11F09 23*03-VL-VH-Fc (SEQ ID NO:286) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:183, which includes a heavy chain variable domain of 11F09 23*03 connected to the C-terminus of a light chain variable domain of 11F09 23*03 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:286. In certain embodiments, the TriNKET® comprises an amino acid sequence at least 95%, identical to the amino acid sequence of SEQ ID NO:286. In certain embodiments, the TriNKET® comprises an amino acid sequence at least 96%, identical to the amino acid sequence of SEQ ID NO:286. In certain embodiments, the TriNKET® comprises an amino acid sequence at least 97%, identical to the amino acid sequence of SEQ ID NO:286. In certain embodiments, the TriNKET® comprises an amino acid sequence at least 98%, identical to the amino acid sequence of SEQ ID NO:286. In certain embodiments, the TriNKET® comprises an amino acid sequence at least 99%, identical to the amino acid sequence of SEQ ID NO:286. In certain embodiments, the TriNKET® comprises the amino acid sequence of SEQ ID NO:286.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-11F09 23*03-VL-VH-Fc. In A49MI-VH-CH1-Fc, the Fc domain also includes K360E and K409W substitutions for heterodimerization with the Fc in scFv-11F09 11F09 23*03-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1891-VL-VH TriNKET®. AB1891-TriNKET® includes (a) a 5T4-binding scFv sequence comprising the VH and VL sequences of the 11F09 11*01-BM1 scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1891-TriNKET® includes three polypeptides: scFv-11F09 11*01-BM1-VL-VH-Fc (SEQ ID NO:287), A49MI-VH-CH1-Fc (SEQ ID NO:199), and A49MI-VL-CL (SEQ ID NO:200).

scFv-11F09 23*03-VL-VH-Fc (“Chain S”)
(SEQ ID NO: 287)
DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVKPGGSLR
LSCAASGFTFSDYGMHWIRQAPGKCLEWVSYISSGSSTFYYADTVKGRFT
ISRDNAKNSLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG

scFv-11F09 11*01-BM1-VL-VH-Fc (SEQ ID NO:287) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:248, which includes a heavy chain variable domain of 11F09 11*01-BM1 connected to the C-terminus of a light chain variable domain of 11F09 11*01-BM1 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:287. In certain embodiments, the TriNKET® comprises an amino acid sequence at least 95%, identical to the amino acid sequence of SEQ ID NO:287. In certain embodiments, the TriNKET® comprises an amino acid sequence at least 96%, identical to the amino acid sequence of SEQ ID NO:287. In certain embodiments, the TriNKET® comprises an amino acid sequence at least 97%, identical to the amino acid sequence of SEQ ID NO:287. In certain embodiments, the TriNKET® comprises an amino acid sequence at least 98%, identical to the amino acid sequence of SEQ ID NO:287. In certain embodiments, the TriNKET® comprises an amino acid sequence at least 99%, identical to the amino acid sequence of SEQ ID NO:287. In certain embodiments, the TriNKET® comprises the amino acid sequence of SEQ ID NO:287.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-11F09 11*01-BM1-VL-VH-Fc. In A49MI-VH-CH1-Fc, the Fc domain also includes K360E and K409W substitutions for heterodimerization with the Fc in scFv-11F09 11F09 11*01-BM1-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB1893-VL-VH TriNKET®. AB1893-TriNKET® includes (a) a 5T4-binding scFv sequence comprising the VH and VL sequences of the 11F09 23*03-BM2 scFv described in Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1893-TriNKET® includes three polypeptides: scFv-11F09 23*03-BM2-VL-VH-Fc (SEQ ID NO:249), A49MI-VH-CH1-Fc (SEQ ID NO:199), and A49MI-VL-CL (SEQ ID NO:200).

scFv-11F09 23*03-BM2-VL-VH-Fc (“Chain S”)
(SEQ ID NO: 249)
DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLLESGGGLVQPGGSLR
LSCAASGFTFSDYGMHWVRQAPGKCLEWLAYISSGSSTFYYADTVKGRFT
ISRDNAKNTLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG

scFv-11F09 23*03-BM2-VL-VH-Fc (SEQ ID NO:249) represents the full sequence of a 5T4 binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:251, which includes a heavy chain variable domain of 11F09 23*03-BM2 connected to the C-terminus of a light chain variable domain of 11F09 23*03-BM2 via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:249. In certain embodiments, the TriNKET® comprises an amino acid sequence at least 95%, identical to the amino acid sequence of SEQ ID NO:249. In certain embodiments, the TriNKET® comprises an amino acid sequence at least 96%, identical to the amino acid sequence of SEQ ID NO:249. In certain embodiments, the TriNKET® comprises an amino acid sequence at least 97%, identical to the amino acid sequence of SEQ ID NO:249. In certain embodiments, the TriNKET® comprises an amino acid sequence at least 98%, identical to the amino acid sequence of SEQ ID NO:249. In certain embodiments, the TriNKET® comprises an amino acid sequence at least 99%, identical to the amino acid sequence of SEQ ID NO:249. In certain embodiments, the TriNKET® comprises the amino acid sequence of SEQ ID NO:249.

A49MI-VH-CH1-Fc (SEQ ID NO:199) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-11F09 23*03-BM2-VL-VH-Fc. In A49MI-VH-CH1-Fc, the Fc domain also includes K360E and K409W substitutions for heterodimerization with the Fc in scFv-11F09 11F09 23*03-BM2-VL-VH-Fc.

A49MI-VL-CL (SEQ ID NO:200) represents the light chain portion of the Fab fragment comprising a light chain variable domain of NKG2D-binding A49MI (SEQ ID NO:85) and a light chain constant domain.

Another TriNKET® described in the present disclosure is AB2509-F3-TriNKET®. AB2509-F3-TriNKET® includes (a) an NKG2D-binding scFv sequence comprising the VH and VL sequences of A49MI scFv described in Table 2, in the orientation of VH positioned C-terminal to VL, linked to an Fc domain and (b) a 5T4-binding Fab fragment derived from 05H04, including a heavy chain portion comprising a heavy chain variable domain and a CH1 domain, and a light chain portion comprising a light chain variable domain and a light chain constant domain, wherein the CH1 domain is connected to the Fc domain. AB2509-F3-TriNKET® includes three polypeptides: scFv-A49MI-VL-VH-Fc (SEQ ID NO:252), 05H04-VH-CH1-Fc (SEQ ID NO:253), and 05H04-VL-CL (SEQ ID NO:254).

scFv-A49MI-VL-VH-Fc (“Chain S”)
(SEQ ID NO: 252)
DIQMTQSPSSVSASVGDRVTITCRASQGISSWLAWYQQKPGKAPKLLIYA
ASSLQSGVPSRESGSGSGTDFTLTISSLQPEDFATYYCQQGVSFPRTFGC
GTKVEIKGGGGSGGGGSGGGGSGGGGSEVQLVESGGGLVKPGGSLRLSCA
ASGFTFSSYSMNWVRQAPGKCLEWVSSISSSSSYIYYADSVKGRFTISRD
NAKNSLYLQMNSLRAEDTAVYYCARGAPIGAAAGWFDPWGQGTLVTVSSG
SDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPRVYTLPPCRDELTKNQVSLTCLV
KGFYPSDIAVEWESNGQPENNYKTTPPVLVSDGSFTLYSKLTVDKSRWQQ
GNVFSCSVMHEALHNHYTQKSLSLSPG
05H04-VH-CH1-FC (“Chain H”)
(SEQ ID NO: 253)
QVQLVQSGAEVKKPGASVKVSCKASGYKFTDYYMDWVRQAPGQGLEWIGY
IFPNDASTTYNEKFKGKATLTADKSTNTAYMELSSLRSEDTAVYYCARSR
DADYWGQGTTVTVSSAS TKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVCTL
PPSRDELTENQVSLTCLVKGFYPSDIAVEWESNGQPENNYKTTPPVLDSD
GSFFLYSWLTVDKSRWQQGNVFSCSVMHEALHNHYTQKSLSLSPG
05H04-VL-CL (“Chain L”)
(SEQ ID NO: 254)
ENVLTQSPATLSLSPGERATLSCSAKSSVSYIHWYQQKPGQAPRLLIYDT
SYLGSGIPARESGSGSGTDYTLTISSLEPEDFAVYYCQQWSSYPYTFGGG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

scFv-A49MI-VL-VH-Fc (SEQ ID NO:252) represents the full sequence of an NKG2D binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in 05H04-VH-CH1-Fc as described below. The scFv has the amino acid sequence of SEQ ID NO:288, which includes a heavy chain variable domain of A49MI connected to the C-terminus of a light chain variable domain of A49MI via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:252. In certain embodiments, the TriNKET® comprises an amino acid sequence at least 95%, identical to the amino acid sequence of SEQ ID NO:252. In certain embodiments, the TriNKET® comprises an amino acid sequence at least 96%, identical to the amino acid sequence of SEQ ID NO:252. In certain embodiments, the TriNKET® comprises an amino acid sequence at least 97%, identical to the amino acid sequence of SEQ ID NO:252. In certain embodiments, the TriNKET® comprises an amino acid sequence at least 98%, identical to the amino acid sequence of SEQ ID NO:252. In certain embodiments, the TriNKET® comprises an amino acid sequence at least 99%, identical to the amino acid sequence of SEQ ID NO:252. In certain embodiments, the TriNKET® comprises the amino acid sequence of SEQ ID NO:252.

05H04-VH-CH1-Fc (SEQ ID NO:253) represents the heavy chain portion of the Fab fragment, which comprises a heavy chain variable domain of 5T4-binding 05H04 (SEQ ID NO:150) and a CH1 domain, connected to an Fc domain. The Fc domain in 05H04-VH-CH1-Fc includes a Y349C substitution in the CH3 domain, which forms a disulfide bond with an S354C substitution on the Fc in scFv-A49MI-VL-VH-Fc. In 05H04-VH-CH1-Fc, the Fc domain also includes K360E and K409W substitutions for heterodimerization with the Fc in scFv-A49MI-VL-VH-Fc.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:253. In certain embodiments, the TriNKET® comprises an amino acid sequence at least 95%, identical to the amino acid sequence of SEQ ID NO:253. In certain embodiments, the TriNKET® comprises an amino acid sequence at least 96%, identical to the amino acid sequence of SEQ ID NO:253. In certain embodiments, the TriNKET® comprises an amino acid sequence at least 97%, identical to the amino acid sequence of SEQ ID NO:253. In certain embodiments, the TriNKET® comprises an amino acid sequence at least 98%, identical to the amino acid sequence of SEQ ID NO:253. In certain embodiments, the TriNKET® comprises an amino acid sequence at least 99%, identical to the amino acid sequence of SEQ ID NO:253. In certain embodiments, the TriNKET® comprises the amino acid sequence of SEQ ID NO:253.

05H04-VL-CL (SEQ ID NO:254) represents the light chain portion of the Fab fragment comprising a light chain variable domain of 5T4-binding 05H04 (SEQ ID NO:151) and a light chain constant domain.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:254. In certain embodiments, the TriNKET® comprises an amino acid sequence at least 95%, identical to the amino acid sequence of SEQ ID NO:254. In certain embodiments, the TriNKET® comprises an amino acid sequence at least 96%, identical to the amino acid sequence of SEQ ID NO:254. In certain embodiments, the TriNKET® comprises an amino acid sequence at least 97%, identical to the amino acid sequence of SEQ ID NO:254. In certain embodiments, the TriNKET® comprises an amino acid sequence at least 98%, identical to the amino acid sequence of SEQ ID NO:254. In certain embodiments, the TriNKET® comprises an amino acid sequence at least 99%, identical to the amino acid sequence of SEQ ID NO:254. In certain embodiments, the TriNKET® comprises the amino acid sequence of SEQ ID NO:254.

Another TriNKET® described in the present disclosure is AB2511-F3-TriNKET®. AB2511-F3-TriNKET® includes (a) an NKG2D-binding scFv sequence comprising the VH and VL sequences of A49MI scFv described in Table 1, in the orientation of VH positioned C-terminal to VL, linked to an Fc domain and (b) a 5T4-binding Fab fragment derived from 11F09 21*05, including a heavy chain portion comprising a heavy chain variable domain and a CH1 domain, and a light chain portion comprising a light chain variable domain and a light chain constant domain, wherein the CH1 domain is connected to the Fc domain. AB2511-F3-TriNKET® includes three polypeptides: scFv-A49MI-VL-VH-Fc (SEQ ID NO:252), 11F09 21*05-VH-CH1-Fc (SEQ ID NO:255), and 11F09 21*05-VL-CL (SEQ ID NO:256).

11F09 21*05-VH-CH1-Fc (“Chain H”)
(SEQ ID NO: 255)
EVQLVESGGGLVKPGGSLRLSCEASGFTFSDYGMHWVRQAPGKGLEWLAY
ISSGSSTFYYADTVKGRFTISRDNAKNSLYLQMNSLRAEDTAVYYCGSSQ
SYYRGTMDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VCTLPPSRDELTENQVSLTCLVKGFYPSDIAVEWESNGQPENNYKTTPPV
LDSDGSFFLYSWLTVDKSRWQQGNVFSCSVMHEALHNHYTQKSLSLSPG
11F09 21*05-VL-CL (“Chain L”)
(SEQ ID NO: 256)
DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

scFv-A49MI-VL-VH-Fc (SEQ ID NO:252) represents the full sequence of an NKG2D binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in 11F09 21*05-VH-CH1-Fc as described below. The scFv has the amino acid sequence of SEQ ID NO:288, which includes a heavy chain variable domain of A49MI connected to the C-terminus of a light chain variable domain of A49MI via a (G₄S)₄ linker (SEQ ID NO:119). The scFv comprises substitutions of Cys in the VH and VL regions at G44 and G100, facilitating formation of a disulfide bridge between the VH and VL of the scFv.

11F09 21*05-VH-CH1-Fc (SEQ ID NO:255) represents the heavy chain portion of the Fab fragment, which comprises a heavy chain variable domain of 5T4-binding 11F09 21*05 (SEQ ID NO:161) and a CH1 domain, connected to an Fc domain. The Fc domain in 11F09 21*05-VH-CH1-Fc includes a Y349C substitution in the CH3 domain, which forms a disulfide bond with an S354C substitution on the Fc in scFv-A49MI-VL-VH-Fc. In 11F09 21*05-VH-CH1-Fc, the Fc domain also includes K360E and K409W substitutions for heterodimerization with the Fc in scFv-A49MI-VL-VH-Fc.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:255. In certain embodiments, the TriNKET® comprises an amino acid sequence at least 95%, identical to the amino acid sequence of SEQ ID NO:255. In certain embodiments, the TriNKET® comprises an amino acid sequence at least 96%, identical to the amino acid sequence of SEQ ID NO:255. In certain embodiments, the TriNKET® comprises an amino acid sequence at least 97%, identical to the amino acid sequence of SEQ ID NO:255. In certain embodiments, the TriNKET® comprises an amino acid sequence at least 98%, identical to the amino acid sequence of SEQ ID NO:255. In certain embodiments, the TriNKET® comprises an amino acid sequence at least 99%, identical to the amino acid sequence of SEQ ID NO:255. In certain embodiments, the TriNKET® comprises the amino acid sequence of SEQ ID NO:255.

11F09 21*05-VL-CL (SEQ ID NO:256) represents the light chain portion of the Fab fragment comprising a light chain variable domain of 5T4-binding 11F09 21*05 (SEQ ID NO:177) and a light chain constant domain.

Another TriNKET® described in the present disclosure is AB2512-F3-TriNKET®. AB2512-F3-TriNKET® includes (a) an NKG2D-binding scFv sequence comprising the VH and VL sequences of A49MI scFv described in Table 2, in the orientation of VH positioned C-terminal to VL, linked to an Fc domain and (b) a 5T4-binding Fab fragment derived from 11F09, including a heavy chain portion comprising a heavy chain variable domain and a CH1 domain, and a light chain portion comprising a light chain variable domain and a light chain constant domain, wherein the CH1 domain is connected to the Fc domain. AB2512-F3-TriNKET® includes three polypeptides: scFv-A49MI-VL-VH-Fc (SEQ ID NO:252), 11F09-VH-CH1-Fc (SEQ ID NO:257), and 11F09-VL-CL (SEQ ID NO:258).

11F09-VH-CH1-Fc (“Chain H”)
(SEQ ID NO: 257)
EVQLVESGGGLVKPGGSLRLSCEASGFTFSDYGMHWVRQAPGKGLEWLAY
ISSGSSTFYYADTVKGRFTISRDNAKNSLYLQMNSLRAEDTAVYYCGSSQ
SYYRGTMDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VCTLPPSRDELTENQVSLTCLVKGFYPSDIAVEWESNGQPENNYKTTPPV
LDSDGSFFLYSWLTVDKSRWQQGNVFSCSVMHEALHNHYTQKSLSLSPG
11F09-VL-CL (“Chain L”)
(SEQ ID NO: 258)
DIQLTQSPSSLSASVGDRVTITCRASQGVSTSTYTYLHWFQQKPGKAPKL
LIKFASNLESGVPSRFSGSGSGTDFTLTISSLQPEDFATYYCQHSWEIPW
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

scFv-A49MI-VL-VH-Fc (SEQ ID NO:252) represents the full sequence of an NKG2D binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in 11F09-VH-CH1-Fc as described below. The scFv has the amino acid sequence of SEQ ID NO:288, which includes a heavy chain variable domain of A49MI connected to the C-terminus of a light chain variable domain of A49MI via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

11F09-VH-CH1-Fc (SEQ ID NO:257) represents the heavy chain portion of the Fab fragment, which comprises a heavy chain variable domain of 5T4-binding 11F09 (SEQ ID NO:161) and a CH1 domain, connected to an Fc domain. The Fc domain in 11F09-VH-CH1-Fc includes a Y349C substitution in the CH3 domain, which forms a disulfide bond with an S354C substitution on the Fc in scFv-A49MI-VL-VH-Fc. In 11F09-VH-CH1-Fc, the Fc domain also includes K360E and K409W substitutions for heterodimerization with the Fc in scFv-A49MI-VL-VH-Fc.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:257. In certain embodiments, the TriNKET® comprises an amino acid sequence at least 95%, identical to the amino acid sequence of SEQ ID NO:257. In certain embodiments, the TriNKET® comprises an amino acid sequence at least 96%, identical to the amino acid sequence of SEQ ID NO:257. In certain embodiments, the TriNKET® comprises an amino acid sequence at least 97%, identical to the amino acid sequence of SEQ ID NO:257. In certain embodiments, the TriNKET® comprises an amino acid sequence at least 98%, identical to the amino acid sequence of SEQ ID NO:257. In certain embodiments, the TriNKET® comprises an amino acid sequence at least 99%, identical to the amino acid sequence of SEQ ID NO:257. In certain embodiments, the TriNKET® comprises the amino acid sequence of SEQ ID NO:257.

11F09-VL-CL (SEQ ID NO:258) represents the light chain portion of the Fab fragment comprising a light chain variable domain of 5T4-binding 11F09 (SEQ ID NO:162) and a light chain constant domain.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:258. In certain embodiments, the TriNKET® comprises an amino acid sequence at least 95%, identical to the amino acid sequence of SEQ ID NO:258. In certain embodiments, the TriNKET® comprises an amino acid sequence at least 96%, identical to the amino acid sequence of SEQ ID NO:258. In certain embodiments, the TriNKET® comprises an amino acid sequence at least 97%, identical to the amino acid sequence of SEQ ID NO:258. In certain embodiments, the TriNKET® comprises an amino acid sequence at least 98%, identical to the amino acid sequence of SEQ ID NO:258. In certain embodiments, the TriNKET® comprises an amino acid sequence at least 99%, identical to the amino acid sequence of SEQ ID NO:258. In certain embodiments, the TriNKET® comprises the amino acid sequence of SEQ ID NO:258.

Another TriNKET® described in the present disclosure is AB2513-F3-TriNKET®. AB2513-F3-TriNKET® includes (a) an NKG2D-binding scFv sequence comprising the VH and VL sequences of A49MI scFv described in Table 1, in the orientation of VH positioned C-terminal to VL, linked to an Fc domain and (b) a 5T4-binding Fab fragment derived from 10F10 21*05, including a heavy chain portion comprising a heavy chain variable domain and a CH1 domain, and a light chain portion comprising a light chain variable domain and a light chain constant domain, wherein the CH1 domain is connected to the Fc domain. AB2513-F3-TriNKET® includes three polypeptides: scFv-A49MI-VL-VH-Fc (SEQ ID NO:252), 10F10 21*05-VH-CH1-Fc (SEQ ID NO:259), and 10F10 21*05-VL-CL (SEQ ID NO:260).

10F10 21*05-VH-CH1-Fc (“Chain H”)
(SEQ ID NO: 259)
EVQLVESGGGLVKPGGSLRLSCAASRFTFSDFGMHWVRQAPGKGLEWVSY
ISSGSSTIYYADTVKGRFTISRDNAKNSLYLQMNSLRAEDTAVYYCASSQ
SYYRGTLDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VCTLPPSRDELTENQVSLTCLVKGFYPSDIAVEWESNGQPENNYKTTPPV
LDSDGSFFLYSWLTVDKSRWQQGNVFSCSVMHEALHNHYTQKSLSLSPG
10F10 21*05-VL-CL (“Chain L”)
(SEQ ID NO: 260)
DIQLTQSPSSLSASVGDRVTITCRASQSVTTSNYNYMHWFQQKPGKAPKL
LIKFASNLESGVPSRFSGSGSGTDFTLTISSLQPEDFATYYCQHSWEIPW
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

scFv-A49MI-VL-VH-Fc (SEQ ID NO:252) represents the full sequence of an NKG2D binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in 10F10-VH-CH1-Fc as described below. The scFv has the amino acid sequence of SEQ ID NO:288, which includes a heavy chain variable domain of A49MI connected to the C-terminus of a light chain variable domain of A49MI via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

10F10 21*05-VH-CH1-Fc (SEQ ID NO:259) represents the heavy chain portion of the Fab fragment, which comprises a heavy chain variable domain of 5T4-binding 10F10 21*05 (SEQ ID NO:166) and a CH1 domain, connected to an Fc domain. The Fc domain in 10F10-VH-CH1-Fc includes a Y349C substitution in the CH3 domain, which forms a disulfide bond with an S354C substitution on the Fc in scFv-A49MI-VL-VH-Fc. In 10F10-VH-CH1-Fc, the Fc domain also includes K360E and K409W substitutions for heterodimerization with the Fc in scFv-A49MI-VL-VH-Fc.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:259. In certain embodiments, the TriNKET® comprises an amino acid sequence at least 95%, identical to the amino acid sequence of SEQ ID NO:259. In certain embodiments, the TriNKET® comprises an amino acid sequence at least 96%, identical to the amino acid sequence of SEQ ID NO:259. In certain embodiments, the TriNKET® comprises an amino acid sequence at least 97%, identical to the amino acid sequence of SEQ ID NO:259. In certain embodiments, the TriNKET® comprises an amino acid sequence at least 98%, identical to the amino acid sequence of SEQ ID NO:259. In certain embodiments, the TriNKET® comprises an amino acid sequence at least 99%, identical to the amino acid sequence of SEQ ID NO:259. In certain embodiments, the TriNKET® comprises the amino acid sequence of SEQ ID NO:259.

1010 21*05-VL-CL (SEQ ID NO:260) represents the light chain portion of the Fab fragment comprising a light chain variable domain of 5T4-binding 10F10 21*05 (SEQ ID NO:145) and a light chain constant domain.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:260. In certain embodiments, the TriNKET® comprises an amino acid sequence at least 95%, identical to the amino acid sequence of SEQ ID NO:260. In certain embodiments, the TriNKET® comprises an amino acid sequence at least 96%, identical to the amino acid sequence of SEQ ID NO:260. In certain embodiments, the TriNKET® comprises an amino acid sequence at least 97%, identical to the amino acid sequence of SEQ ID NO:260. In certain embodiments, the TriNKET® comprises an amino acid sequence at least 98%, identical to the amino acid sequence of SEQ ID NO:260. In certain embodiments, the TriNKET® comprises an amino acid sequence at least 99%, identical to the amino acid sequence of SEQ ID NO:260. In certain embodiments, the TriNKET® comprises the amino acid sequence of SEQ ID NO:260.

Another TriNKET® described in the present disclosure is AB1002-F3-TriNKET®. AB1002-F3-TriNKET® includes (a) an NKG2D-binding scFv sequence comprising the VH and VL sequences of A49MI scFv described in Table 1, in the orientation of VH positioned C-terminal to VL, linked to an Fc domain and (b) a 5T4-binding Fab fragment derived from AB1002, including a heavy chain portion comprising a heavy chain variable domain and a CH1 domain, and a light chain portion comprising a light chain variable domain and a light chain constant domain, wherein the CH1 domain is connected to the Fc domain. AB1002-F3-TriNKET® includes three polypeptides: scFv-A49MI-VL-VH-Fc (SEQ ID NO:252), AB1002-VH-CH1-Fc (SEQ ID NO:261), and 10F10-VL-CL (SEQ ID NO:262).

AB1002-VH-CH1-FC (“Chain H”)
(SEQ ID NO: 261)
EVQLVESGGGLVKPGGSLRLSCAASRFTFSDFGMHWVRQAPGKGLEWVSY
ISSGSSTIYYADSVKGRFTISRDNAKNSLYLQMNSLRAEDTAVYYCASSQ
SYYRGTLDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VCTLPPSRDELTENQVSLTCLVKGFYPSDIAVEWESNGQPENNYKTTPPV
LDSDGSFFLYSWLTVDKSRWQQGNVFSCSVMHEALHNHYTQKSLSLSPG
AB1002-VL-CL (“Chain L”)
(SEQ ID NO: 262)
DIQLTQSPSSLSASVGDRVTITCRASQSVTTSNYNYMHWFQQKPGKAPKL
LIKFASNLESGVPSRFSGSGSGTDFTLTISSLQPEDFATYYCQHSWEIPW
TFGGGTKVEIKRTVAAPSVFIFPPSDEQLKSGTASVVCLLNNEYPREAKV
QWKVDNALQSGNSQESVTEQDSKDSTYSLSSTLTLSKADYEKHKVYACEV
THQGLSSPVTKSFNRGEC

scFv-A49MI-VL-VH-Fc (SEQ ID NO:252) represents the full sequence of an NKG2D binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in AB1002-VH-CH1-Fc as described below. The scFv has the amino acid sequence of SEQ ID NO:288, which includes a heavy chain variable domain of A49MI connected to the C-terminus of a light chain variable domain of A49MI via a (G₄S)₄ linker (SEQ ID NO:119). The scFv comprises substitutions of Cys in the VH and VL regions at G44 and G100, resulting in G44C and G100C substitutions, facilitating formation of a disulfide bridge between the VH and VL of the scFv.

AB1002-VH-CH1-Fc (SEQ ID NO:261) represents the heavy chain portion of the Fab fragment, which comprises a heavy chain variable domain of 5T4-binding AB1002 (SEQ ID NO:144) and a CH1 domain, connected to an Fc domain. The Fc domain in AB1002-VH-CH1-Fc includes a Y349C substitution in the CH3 domain, which forms a disulfide bond with an S354C substitution on the Fc in scFv-A49MI-VL-VH-Fc. In AB1002-VH-CH1-Fc, the Fc domain also includes K360E and K409W substitutions for heterodimerization with the Fc in scFv-A49MI-VL-VH-Fc.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:261. In certain embodiments, the TriNKET® comprises an amino acid sequence at least 95%, identical to the amino acid sequence of SEQ ID NO:261. In certain embodiments, the TriNKET® comprises an amino acid sequence at least 96%, identical to the amino acid sequence of SEQ ID NO:261. In certain embodiments, the TriNKET® comprises an amino acid sequence at least 97%, identical to the amino acid sequence of SEQ ID NO:261. In certain embodiments, the TriNKET® comprises an amino acid sequence at least 98%, identical to the amino acid sequence of SEQ ID NO:261. In certain embodiments, the TriNKET® comprises an amino acid sequence at least 99%, identical to the amino acid sequence of SEQ ID NO:261. In certain embodiments, the TriNKET® comprises the amino acid sequence of SEQ ID NO:261.

AB1002-VL-CL (SEQ ID NO:262) represents the light chain portion of the Fab fragment comprising a light chain variable domain of 5T4-binding AB1002 (SEQ ID NO:145) and a light chain constant domain.

In certain embodiments, the TriNKET® provided herein comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:266. In certain embodiments, the TriNKET® comprises an amino acid sequence at least 95%, identical to the amino acid sequence of SEQ ID NO:266. In certain embodiments, the TriNKET® comprises an amino acid sequence at least 96%, identical to the amino acid sequence of SEQ ID NO:266. In certain embodiments, the TriNKET® comprises an amino acid sequence at least 97%, identical to the amino acid sequence of SEQ ID NO:266. In certain embodiments, the TriNKET® comprises an amino acid sequence at least 98%, identical to the amino acid sequence of SEQ ID NO:266. In certain embodiments, the TriNKET® comprises an amino acid sequence at least 99%, identical to the amino acid sequence of SEQ ID NO:266. In certain embodiments, the TriNKET® comprises the amino acid sequence of SEQ ID NO:266.

In certain embodiments, an F3′ TriNKET® described in the present disclosure is identical to one of the exemplary TriNKETs® described above, except that (a) the Fc domain linked to the NKG2D-binding Fab fragment includes Q347R, D399V, and F405T substitutions in the CH3 domain for heterodimerization, and the Fc domain linked to the 5T4-binding scFv includes matching K360E and K409W substitution in the CH3 domain; and/or (b) the Fc domain linked to the NKG2D-binding Fab fragment includes an S354C substitution in the CH3 domain, and the Fc domain linked to the 5T4-binding scFv includes a matching Y349C substitution in the CH3 domain for forming a disulfide bond.

In certain embodiments, a 2-Fab TriNKET® described in the present disclosure is identical to one of the exemplary TriNKETs® described above, except that the Fc domain linked to the NKG2D-binding Fab fragment includes a F405L substitution in the CH3 domain for heterodimerization, and the Fc domain linked to the 5T4-binding Fab fragment includes a matching K409R substitution in the CH3 domain.

In certain embodiments, a TriNKET® described herein comprises a human NKG2D-binding site which is a Fab fragment comprising a VH and VL, wherein the VH comprises CDR1, CDR2, and CDR3 sequences represented by the amino acid sequences of SEQ ID NOs: 81, 82, and 97, respectively, and the VL comprises CDR1, CDR2, and CDR3 sequences represented by the amino acid sequences of SEQ ID NOs: 86, 77, and 87, respectively. In certain embodiments, a TriNKET® described herein comprises a human 5T4-binding site which is an scFv comprising a VH and a VL, wherein the VH comprises CDR1, CDR2, and CDR3 sequences comprising the amino acid sequences of SEQ ID NOs: 138, 139, and 140, respectively, and the VL comprises CDR1, CDR2, and CDR3 sequences comprising the amino acid sequences of SEQ ID NOs: 141, 142, and 143, respectively, wherein the VH is positioned at the C-terminus of the VL, wherein the VH is linked to the VL via a flexible linker comprising (G₄S)₄ (SEQ ID NO:119), and wherein a disulfide bridge is formed between C44 of the VH and C100 of the VL, numbered under the Kabat numbering scheme. In certain embodiments, a TriNKET® described herein comprises a human CD16a-binding site which is a human IgG1 antibody Fc domain comprising: (i) a first Fc domain polypeptide that comprises an amino acid sequence at least 95% identical to SEQ ID NO:118 and comprising Y349C, K360E, and K409W substitutions relative to SEQ ID NO:118, numbered according to the EU numbering system, and (ii) a second Fc domain polypeptide that comprises an amino acid sequence at least 95% identical to SEQ ID NO:118 and comprising Q347R, S354C, D399V and F405T substitutions relative to SEQ ID NO:118, numbered according to the EU numbering system, wherein a disulfide bridge is formed between C349 of (i) and C354 of (ii), and wherein (i) and (iii) form a heterodimer, wherein the VH of (a) is fused to the N-terminus of the first Fc domain polypeptide, and the VH of (b) is fused to the N-terminus of the second Fc domain polypeptide via a hinge comprising Ala-Ser. In some embodiments, for a TriNKET® having the features described herein, the VH of the human NKG2D-binding site comprises the amino acid sequence of SEQ ID NO:95, and the VL of the human NKG2D-binding site comprises the amino acid sequence of SEQ ID NO:85. In some embodiments, for a TriNKET® having the features described herein, the VH of the human 5T4-binding site comprises the amino acid sequence of SEQ ID NO:146 and the VL of the human 5T4-binding site comprises the amino acid sequence of SEQ ID NO:147. In some embodiments, for a TriNKET® having the features described herein, the first Fc domain polypeptide comprises an amino acid sequence at least 98% identical to SEQ ID NO:118, and the second Fc domain polypeptide comprises an amino acid sequence at least 98% identical to SEQ ID NO:118. In some embodiments, for a TriNKET® having the features described herein, the scFV comprising a VH and a VL comprises the amino acid sequence of SEQ ID NO:148. In some embodiments, a TriNKET® having the features described herein comprises a first polypeptide, a second polypeptide and a third polypeptide, wherein the first polypeptide comprising the amino acid sequence of SEQ ID NO:198; the second polypeptide comprising the amino acid sequence of SEQ ID NO:199; and the third polypeptide comprising the amino acid sequence of SEQ ID NO:200.

A skilled person in the art would appreciate that during production and/or storage of proteins, N-terminal glutamate (E) or glutamine (Q) can be cyclized to form a lactam (e.g., spontaneously or catalyzed by an enzyme present during production and/or storage). Accordingly, in some embodiments where the N-terminal residue of an amino acid sequence of a polypeptide is E or Q, a corresponding amino acid sequence with the E or Q replaced with pyroglutamate is also contemplated herein.

A skilled person in the art would also appreciate that during protein production and/or storage, the C-terminal lysine (K) of a protein can be removed (e.g., spontaneously or catalyzed by an enzyme present during production and/or storage). Such removal of K is often observed with proteins that comprise an Fc domain at its C-terminus. Accordingly, in some embodiments where the C-terminal residue of an amino acid sequence of a polypeptide (e.g., an Fc domain sequence) is K, a corresponding amino acid sequence with the K removed is also contemplated herein.

The multispecific binding proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells or chromosomally integrated into the genome of host cells to produce the multimeric proteins.

To achieve the highest yield of the multispecific binding protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multispecific binding protein. The multispecific binding proteins can be isolated and purified. Such multispecific binding proteins that have been isolated and purified, in some embodiments, are substantially free of at least one component as compared to the multispecific binding protein produced in the culture. Therefore, a purified multispecific binding protein can be partly or completely separated from one or more other substances as it is generated, stored or subsisted in non-naturally occurring environments. The multispecific binding proteins can be isolated and purified from a cell culture using methods known in the art including centrifugation, depth filtration, cell lysis, homogenization, freeze-thawing, affinity purification, gel filtration, ammonium sulfate or ethanol precipitation, ion exchange chromatography (anion or cation), hydrophobic interaction exchange chromatography, and mixed-mode chromatography. Other well-known methods are described in Process Scale Purification of Antibodies, Second Edition, U. Gottschalk (Ed.), John Wiley & Sons, Inc., Hoboken, N.J. (2017). Alternatively, the multispecific binding proteins provided herein can be obtained using well-known recombinant methods (see, for example, Sambrook et al., Molecular Cloning: A Laboratory Manual, Third Ed., Cold Spring Harbor Laboratory, New York (2001); and Ausubel et al., Current Protocols in Molecular Biology, John Wiley & Sons, Baltimore, MID (1999)). The methods and conditions for purification of the multispecific binding proteins provided herein can be chosen by those skilled in the art, and purification monitored, for example, by a binding and/or functional assay as described herein.

The present disclosure also provides for nucleic acids encoding one or more of the chains comprising a multispecific binding protein as described herein. In some embodiments, the nucleic acid encodes a VH that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 1. In some embodiments, the nucleic acid encodes a VL that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in Table 1. In some embodiments, the nucleic acid encodes a VH that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 2. In some embodiments, the nucleic acid encodes a VL that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in Table 2. In some embodiments, the nucleic acid encodes one or more of the chains of the exemplary multispecific binding proteins described herein. Accordingly, in some embodiments, the nucleic acid encodes one or more of Chain S, Chain H, and Chain L described herein. In some embodiments, the nucleic acid encodes a Chain S. In some embodiments, the nucleic acid encodes a Chain H. In some embodiments, the nucleic acid encodes a Chain S. In some embodiments, the nucleic acid encodes a Chain S and a Chain H. In some embodiments, the nucleic acid encodes a Chain S and Chain L. In some embodiments, the nucleic acid encodes a Chain H and Chain L. In some embodiments, the nucleic acid encodes a Chain S, a Chain H, and Chain L. The present disclosure still further provides for nucleic acids encoding an Fc domain or portion thereof as described herein, including chains forming any one of the TriNKET forms described herein.

Exemplary nucleic acid sequences of the present disclosure are provided in Table 11. AB1310, as used herein, refers to a multispecific binding protein comprising the AB1002 scFv (VL-VH). AB1783, as used herein, refers to a multispecific binding protein comprising the AB1002 scFv (VL-VH). The amino acid sequences of AB1310 and AB1783 are identical. The nucleic acid sequences, as shown in Table 11, are distinct for expression in different host cells. AB1783 was optimized for expression in CHO cells.

TABLE 11
Sequences of Exemplary Nucleic Acid Sequences
Encoding Antigen-Binding Sites that Bind 5T4

| Clone | Sequence |
|---|---|
| AB1310 | GTTAGGCGTTTTGCGCTGCTTCGCGATGTACGGGCCAGATATACGCGTTGACATT |
| 5T4-scFv | GATTATTGAGTAGTTATTAATAGTAATCAATTACGGGGTCATTAGTTCATAGCCC |
| (scFv- | ATATATGGAGTTCCGCGTTACATAACTTACGGTAAATGGCCCGCCTGGCTGACCG |
| AB1002- | CCCAACGACCCCCGCCCATTGACGTCAATAATGACGTATGTTCCCATAGTAACGC |
| VL-VH- | CAATAGGGACTTTCCATTGACGTCAATGGGTGGAGTATTTACGGTAAACTGCCCA |
| Fc) | CTTGGGAGTAGATCAAGTGTATCATATGCCAAGTACGCCCCCTATTGAGGTCAAT |
| | GACGGTAAATGGCCCGCCTGGCATTATGCCCAGTACATGACCTTATGGGACTTTC |
| | CTACTTGGCAGTACATCTACGTATTAGTCATCGCTATTACCATGGTGATGCGGTT |
| | TTGGCAGTACATCAATGGGCGTGGATAGCGGTTTGACTCACGGGGATTTCCAAGT |
| | CTCCACCCCATTGACGTCAATGGGAGTTTGTTTTGGCACCAAAATCAACGGGACT |
| | TTCCAAAATGTCGTAACAACTCCGCCCCATTGACGCAAATGGGCGGTAGGCGTGT |
| | ACGGTGGGAGGTCTATATAAGCAGAGCTCGTTTAGTGAACCGTCAGATCGCCTGG |
| | AGACGCCATCCACGCTGTTTTGACCTCCATAGAAGACACCGGGACCGATCCAGCC |
| | TCCGGACTCTAGAGGATCGAACCCTTAAGCTTGCCACCATGGACATGAGAGTGCC |
| | CGCTCAACTGCTGGGACTGCTGCTGCTTTGGCTTCCTGGCGCTAGATGCGACATC |
| | CAGCTGACACAGAGCCCTAGCAGCCTGTCTGCCTCTGTGGGCGACAGAGTGACCA |
| | TCACCTGTAGAGCCAGCCAGAGCGTGACCACCAGCAACTACAACTACATGCACTG |
| | GTTCCAGCAGAAGCCCGGCAAGGCCCCTAAGCTGCTGATCAAGTTCGCCAGCAAC |
| | CTGGAAAGCGGCGTGCCCAGCAGATTTTCTGGCAGCGGCTCTGGCACCGACTTCA |
| | CCCTGACCATATCTAGCCTGCAGCCTGAGGACTTCGCCACCTACTACTGTCAGCA |
| | CAGCTGGGAGATCCCTTGGACCTTTGGCTGTGGCACCAAGGTGGAAATCAAAGGC |
| | GGCGGAGGATCTGGCGGAGGTGGAAGCGGAGGCGGAGGAAGCGGTGGCGGCGGAT |
| | CTGAAGTTCAGCTGGTTGAAAGTGGCGGAGGCCTGGTTAAGCCTGGCGGATCTCT |
| | GAGACTGAGCTGTGCCGCCTCCAGATTCACCTTCAGCGACTTCGGAATGCACTGG |
| | GTCCGACAGGCCCCTGGCAAATGTCTGGAATGGGTGTCCTACATCAGCAGCGGCA |
| | GCAGCACCATCTACTACGCCGATAGCGTGAAGGGCAGATTCACAATCAGCCGGGA |
| | CAACGCCAAGAACAGCCTGTACCTGCAGATGAACTCCCTGAGAGCCGAGGACACC |
| | GCCGTGTACTATTGTGCCAGCAGCCAGTCCTACTACCGGGGCACACTGGATTATT |
| | GGGGCCAGGGCACAACCGTGACAGTGTCCAGCGCCAGCGATAAGACCCACACCTG |
| | TCCTCCATGTCCTGCTCCAGAACTGCTCGGCGGACCTTCCGTGTTCCTGTTTCCT |
| | CCAAAGCCTAAGGACACCCTGATGATCTCCAGAACACCCGAAGTGACCTGCGTGG |
| | TGGTGGATGTGTCTCACGAGGACCCTGAAGTGAAGTTCAATTGGTACGTGGACGG |
| | CGTGGAAGTGCACAATGCCAAGACCAAGCCTAGAGAGGAACAGTACAACAGCACC |
| | TACAGAGTGGTGTCCGTGCTGACCGTGCTGCACCAGGATTGGCTGAACGGCAAAG |
| | AGTACAAGTGCAAGGTGTCCAACAAGGCCCTGCCTGCTCCTATCGAGAAAACCAT |
| | CAGCAAGGCCAAGGGCCAGCCTCGCGAGCCTAGAGTGTATACCTTGCCTCCATGC |
| | CGGGACGAGCTGACCAAGAATCAGGTGTCCCTGACCTGCCTGGTCAAGGGCTTCT |
| | ACCCTTCCGATATCGCCGTGGAATGGGAGAGCAATGGCCAGCCTGAGAACAACTA |
| | CAAGACCACACCTCCTGTGCTGGTGTCCGACGGCAGCTTTACCCTGTACAGCAAG |
| | CTGACAGTGGACAAGAGCAGATGGCAGCAGGGCAACGTGTTCAGCTGCAGCGTGA |
| | TGCACGAGGCCCTGCACAACCACTACACCCAGAAGTCCCTGAGCCTGTCTCCTGG |
| | ATGATAATCTAGAAAGGGTTCGATCCCTACCGGTTAGTAATGAGTTTGATATCTC |
| | GAGAATCAACCTCTGGATTACAAAATTTGTGAAAGATTGACTGGTATTCTTAACT |
| | ATGTTGCTCCTTTTACGCTATGTGGATACGCTGCTTTAATGCCTTTGTATCATGC |
| | TATTGCTTCCCGTATGGCTTTCATTTTCTCCTCCTTGTATAAATCCTGGTTGCTG |
| | TCTCTTTATGAGGAGTTGTGGCCCGTTGTCAGGCAACGTGGCGTGGTGTGCACTG |
| | TGTTTGCTGACGCAACCCCCACTGGTTGGGGCATTGCCACCACCTGTCAGCTCCT |
| | TTCCGGGACTTTCGCTTTCCCCCTCCCTATTGCCACGGCGGAACTCATCGCCGCC |
| | TGCCTTGCCCGCTGCTGGACAGGGGCTCGGCTGTTGGGCACTGACAATTCCGTGG |
| | TGTTGTCGGGGAAGCTGACGTCCTTTCCATGGCTGCTCGCCTGTGTTGCCACCTG |
| | GATTCTGCGCGGGACGTCCTTCTGCTACGTCCCTTCGGCCCTCAATCCAGCGGAC |
| | CTTCCTTCCCGCGGCCTGCTGCCGGCTCTGCGGCCTCTTCCGCGTCTTCGCCTTC |
| | GCCCTCAGACGAGTCGGATCTCCCTTTGGGCCGCCTCCCCGCCTGGAACGGGGGA |
| | GGCTAACTGAAACACGGAAGGAGACAATACCGGAAGGAACCCGCGCTATGACGGC |
| | AATAAAAAGACAGAATAAAACGCACGGGTGTTGGGTCGTTTGTTCATAAACGCGG |
| | GGTTCGGTCCCAGGGCTGGCACTCTGTCGATACCCCACCGAGACCCCATTGGGGC |
| | CAATACGCCCGCGTTTCTTCCTTTTCCCCACCCCACCCCCCAAGTTCGGGTGAAG |
| | GCCCAGGGCTCGCAGCCAACGTCGGGGCGGCAGGCCCTGCCATAGCAGATCTGCG |
| | CAGCTGGGGCTCTAGGGGGTATCCCCACGCGCCCTGTAGCGGCGCATTAAGCGCG |
| | GCGGGTGTGGTGGTTACGCGCAGCGTGACCGCTACACTTGCCAGCGCCCTAGCGC |
| | CCGCTCCTTTCGCTTTCTTCCCTTCCTTTCTCGCCACGTTCGCCGGCTTTCCCCG |
| | TCAAGCTCTAAATCGGGGGCTCCCTTTAGGGTTCCGATTTAGTGCTTTACGGCAC |
| | CTCGACCCCAAAAAACTTGATTAGGGTGATGGTTCACGTAGTGGGCCATCGCCCT |
| | GATAGACGGTTTTTCGCCCTTTGACGTTGGAGTCCACGTTCTTTAATAGTGGACT |
| | CTTGTTCCAAACTGGAACAACACTCAACCCTATCTCGGTCTATTCTTTTGATTTA |
| | TAAGGGATTTTGCCGATTTCGGCCTATTGGTTAAAAAATGAGCTGATTTAACAAA |
| | AATTTAACGCGAATTAATTCTGTGGAATGTGTGTCAGTTAGGGTGTGGAAAGTCC |
| | CCAGGCTCCCCAGCAGGCAGAAGTATGCAAAGCATGCATCTCAATTAGTCAGCAA |
| | CCAGGTGTGGAAAGTCCCCAGGCTCCCCAGCAGGCAGAAGTATGCAAAGCATGCA |
| | TCTCAATTAGTCAGCAACCATAGTCCCGCCCCTAACTCCGCCCATCCCGCCCCTA |
| | ACTCCGCCCAGTTCCGCCCATTCTCCGCCCCATGGCTGACTAATTTTTTTTATTT |
| | ATGCAGAGGCCGAGGCCGCCTCTGCCTCTGAGCTATTCCAGAAGTAGTGAGGAGG |
| | CTTTTTTGGAGGCCTAGGCTTTTGGAAAAAGCTCCCGGGAGCTTGTATATCCATT |
| | TTCGGATCTGATCAAGAGACAGGATGAGGATCGTTTCGCATGATTGAACAAGATG |
| | GATTGCACGCAGGTTCTCCGGCCGCTTGGGTGGAGAGGCTATTCGGCTATGACTG |
| | GGCACAACAGACAATCGGCTGCTCTGATGCCGCCGTGTTCCGGCTGTCAGCGCAG |
| | GGGCGCCCGGTTCTTTTTGTCAAGACCGACCTGTCCGGTGCCCTGAATGAACTGC |
| | AGGACGAGGCAGCGCGGCTATCGTGGCTGGCCACGACGGGCGTTCCTTGCGCAGC |
| | TGTGCTCGACGTTGTCACTGAAGCGGGAAGGGACTGGCTGCTATTGGGCGAAGTG |
| | CCGGGGCAGGATCTCCTGTCATCTCACCTTGCTCCTGCCGAGAAAGTATCCATCA |
| | TGGCTGATGCAATGCGGCGGCTGCATACGCTTGATCCGGCTACCTGCCCATTCGA |
| | CCACCAAGCGAAACATCGCATCGAGCGAGCACGTACTCGGATGGAAGCCGGTCTT |
| | GTCGATCAGGATGATCTGGACGAAGAGCATCAGGGGCTCGCGCCAGCCGAACTGT |
| | TCGCCAGGCTCAAGGCGCGCATGCCCGACGGCGAGGATCTCGTCGTGACCCATGG |
| | CGATGCCTGCTTGCCGAATATCATGGTGGAAAATGGCCGCTTTTCTGGATTCATC |
| | GACTGTGGCCGGCTGGGTGTGGCGGACCGCTATCAGGACATAGCGTTGGCTACCC |
| | GTGATATTGCTGAAGAGCTTGGCGGCGAATGGGCTGACCGCTTCCTCGTGCTTTA |
| | CGGTATCGCCGCTCCCGATTCGCAGCGCATCGCCTTCTATCGCCTTCTTGACGAG |
| | TTCTTCTGAGCGGGACTCTGGGGTTCGCGAAATGACCGACCAAGCGACGCCCAAC |
| | CTGCCATCACGAGATTTCGATTCCACCGCCGCCTTCTATGAAAGGTTGGGCTTCG |
| | GAATCGTTTTCCGGGACGCCGGCTGGATGATCCTCCAGCGCGGGGATCTCATGCT |
| | GGAGTTCTTCGCCCACCCCAACTTGTTTATTGCAGCTTATAATGGTTACAAATAA |
| | AGCAATAGCATCACAAATTTCACAAATAAAGCATTTTTTTCACTGCATTCTAGTT |
| | GTGGTTTGTCCAAACTCATCAATGTATCTTATCATGTCTGTATACCGTCGACCTC |
| | TAGCTAGAGCTTGGCGTAATCATGGTCATAGCTGTTTCCTGTGTGAAATTGTTAT |
| | CCGCTCACAATTCCACACAACATACGAGCCGGAAGCATAAAGTGTAAAGCCTGGG |
| | GTGCCTAATGAGTGAGCTAACTCACATTAATTGCGTTGCGCTCACTGCCCGCTTT |
| | CCAGTCGGGAAACCTGTCGTGCCAGCTGCATTAATGAATCGGCCAACGCGCGGGG |
| | AGAGGCGGTTTGCGTATTGGGCGCTCTTCCGCTTCCTCGCTCACTGACTCGCTGC |
| | GCTCGGTCGTTCGGCTGCGGCGAGCGGTATCAGCTCACTCAAAGGCGGTAATACG |
| | GTTATCCACAGAATCAGGGGATAACGCAGGAAAGAACATGTGAGCAAAAGGCCAG |
| | CAAAAGGCCAGGAACCGTAAAAAGGCCGCGTTGCTGGCGTTTTTCCATAGGCTCC |
| | GCCCCCCTGACGAGCATCACAAAAATCGACGCTCAAGTCAGAGGTGGCGAAACCC |
| | GACAGGACTATAAAGATACCAGGCGTTTCCCCCTGGAAGCTCCCTCGTGCGCTCT |
| | CCTGTTCCGACCCTGCCGCTTACCGGATACCTGTCCGCCTTTCTCCCTTCGGGAA |
| | GCGTGGCGCTTTCTCATAGCTCACGCTGTAGGTATCTCAGTTCGGTGTAGGTCGT |
| | TCGCTCCAAGCTGGGCTGTGTGCACGAACCCCCCGTTCAGCCCGACCGCTGCGCC |
| | TTATCCGGTAACTATCGTCTTGAGTCCAACCCGGTAAGACACGACTTATCGCCAC |
| | TGGCAGCAGCCACTGGTAACAGGATTAGCAGAGCGAGGTATGTAGGCGGTGCTAC |
| | AGAGTTCTTGAAGTGGTGGCCTAACTACGGCTACACTAGAAGAACAGTATTTGGT |
| | ATCTGCGCTCTGCTGAAGCCAGTTACCTTCGGAAAAAGAGTTGGTAGCTCTTGAT |
| | CCGGCAAACAAACCACCGCTGGTAGCGGTGGTTTTTTTGTTTGCAAGCAGCAGAT |
| | TACGCGCAGAAAAAAAGGATCTCAAGAAGATCCTTTGATCTTTTCTACGGGGTCT |
| | GACGCTCAGTGGAACGAAAACTCACGTTAAGGGATTTTGGTCATGAGATTATCAA |
| | AAAGGATCTTCACCTAGATCCTTTTAAATTAAAAATGAAGTTTTAAATCAATCTA |
| | AAGTATATATGAGTAAAGTTGGTCTGACAGTTACCAATGCTTAATGAGTGAGGCA |
| | CCTATCTCAGCGATCTGTCTATTTCGTTCATCCATAGTTGCCTGACTCCCCGTCG |
| | TGTAGATAACTACGATACGGGAGGGCTTACCATCTGGCCCCAGTGCTGCAATGAT |
| | ACCGCGAGACCCACGCTCACCGGCTCCAGATTTATCAGCAATAAACCAGCCAGCC |
| | GGAAGGGCCGAGCGCAGAAGTGGTCCTGCAACTTTATCCGCCTCCATCCAGTCTA |
| | TTAATTGTTGCCGGGAAGCTAGAGTAAGTAGTTCGCCAGTTAATAGTTTGCGCAA |
| | CGTTGTTGCCATTGCTACAGGCATCGTGGTGTCACGCTCGTCGTTTGGTATGGCT |
| | TCATTCAGCTCCGGTTCCCAACGATCAAGGCGAGTTACATGATCCCCCATGTTGT |
| | GCAAAAAAGCGGTTAGCTCCTTCGGTCCTCCGATCGTTGTCAGAAGTAAGTTGGC |
| | CGCAGTGTTATCACTCATGGTTATGGCAGCACTGCATAATTCTCTTACTGTCATG |
| | CCATCCGTAAGATGCTTTTCTGTGACTGGTGAGTACTCAACCAAGTCATTCTGAG |
| | AATAGTGTATGCGGCGACCGAGTTGCTCTTGCCCGGCGTCAATACGGGATAATAC |
| | CGCGCCACATAGCAGAACTTTAAAAGTGCTCATCATTGGAAAACGTTCTTCGGGG |
| | CGAAAACTCTCAAGGATCTTACCGCTGTTGAGATCCAGTTCGATGTAACCCACTC |
| | GTGCACCCAACTGATCTTCAGCATCTTTTACTTTCACCAGCGTTTCTGGGTGAGC |
| | AAAAACAGGAAGGCAAAATGCCGCAAAAAAGGGAATAAGGGCGACACGGAAATGT |
| | TGAATACTCATACTCTTCCTTTTTCAATATTATTGAAGCATTTATCAGGGTTATT |
| | GTCTCATGAGCGGATACATATTTGAATGTATTTAGAAAAATAAACAAATAGGGGT |
| | TCCGCGCACATTTCCCCGAAAAGTGCCACCTGACGTCGACGGATCGGGAGATCTC |
| | CCGATCCCCTATGGTCGACTCTCAGTACAATCTGCTCTGATGCCGCATAGTTAAG |
| | CCAGTATCTGCTCCCTGCTTGTGTGTTGGAGGTCGCTGAGTAGTGCGCGAGCAAA |
| | ATTTAAGCTACAACAAGGCAAGGCTTGACCGACAATTGCATGAAGAATCTGCTTA |
| | GG (SEQ ID NO: 280) |
| AB1310 | GACATCCAGCTGACACAGAGCCCTAGCAGCCTGTCTGCCTCTGTGGGCGACAGAG |
| 5T4-scFv | TGACCATCACCTGTAGAGCCAGCCAGAGCGTGACCACCAGCAACTACAACTAGAT |
| (scFv- | GCACTGGTTCCAGCAGAAGCCCGGCAAGGCCCCTAAGCTGCTGATCAAGTTCGCC |
| AB1002- | AGCAACCTGGAAAGCGGCGTGCCCAGCAGATTTTCTGGCAGCGGCTCTGGCACCG |
| VL-VH- | ACTTCACCCTGACCATATCTAGCCTGCAGCCTGAGGACTTCGCCACCTACTACTG |
| Fc) | TCAGCACAGCTGGGAGATCCCTTGGACCTTTGGCTGTGGCACCAAGGTGGAAATC |
| | AAAGGCGGCGGAGGATCTGGCGGAGGTGGAAGCGGAGGCGGAGGAAGCGGTGGCG |
| | GCGGATCTGAAGTTCAGCTGGTTGAAAGTGGCGGAGGCCTGGTTAAGCCTGGCGG |
| | ATCTCTGAGACTGAGCTGTGCCGCCTCCAGATTCACCTTCAGCGACTTCGGAATG |
| | CACTGGGTCCGACAGGCCCCTGGCAAATGTCTGGAATGGGTGTCCTACATCAGCA |
| | GCGGCAGCAGCACCATCTACTACGCCGATAGCGTGAAGGGCAGATTCACAATCAG |
| | CCGGGACAACGCCAAGAACAGCCTGTACCTGCAGATGAACTCCCTGAGAGCCGAG |
| | GACACCGCCGTGTACTATTGTGCCAGCAGCCAGTCCTACTACCGGGGCACACTGG |
| | ATTATTGGGGCCAGGGCACAACCGTGACAGTGTCCAGCGCCAGCGATAAGACCCA |
| | CACCTGTCCTCCATGTCCTGCTCCAGAACTGCTCGGCGGACCTTCCGTGTTCCTG |
| | TTTCCTCCAAAGCCTAAGGAGACCCTGATGATCTCCAGAACACCCGAAGTGAGCT |
| | GCGTGGTGGTGGATGTGTCTCACGAGGACCCTGAAGTGAAGTTCAATTGGTACGT |
| | GGACGGCGTGGAAGTGCACAATGCCAAGACCAAGCCTAGAGAGGAACAGTACAAC |
| | AGCACCTACAGAGTGGTGTCCGTGCTGACCGTGCTGCACCAGGATTGGCTGAACG |
| | GCAAAGAGTACAAGTGCAAGGTGTCCAACAAGGCCCTGCCTGCTCCTATCGAGAA |
| | AACCATCAGCAAGGCCAAGGGCCAGCCTCGCGAGCCTAGAGTGTATACCTTGCCT |
| | CCATGCCGGGACGAGCTGACCAAGAATCAGGTGTCCCTGACCTGCCTGGTCAAGG |
| | GCTTCTACCCTTCCGATATCGCCGTGGAATGGGAGAGCAATGGCCAGCCTGAGAA |
| | CAACTACAAGACCACACCTCCTGTGCTGGTGTCCGACGGCAGCTTTACCCTGTAC |
| | AGCAAGCTGACAGTGGACAAGAGCAGATGGCAGCAGGGCAACGTGTTCAGCTGCA |
| | GCGTGATGCACGAGGCCCTGCACAACCACTACACCCAGAAGTCCCTGAGCCTGTC |
| | TCCTGGA (SEQ ID NO: 545) |
| AB1310 | GTTAGGCGTTTTGCGCTGCTTCGCGATGTACGGGCCAGATATACGCGTTGACATT |
| A49MI- | GATTATTGACTAGTTATTAATAGTAATCAATTACGGGGTCATTAGTTCATAGCCC |
| VH-CH1- | ATATATGGAGTTCCGCGTTACATAACTTACGGTAAATGGCCCGCCTGGCTGACCG |
| Fc | CCCAACGACCCCCGCCCATTGACGTCAATAATGACGTATGTTCCCATAGTAACGC |
| | CAATAGGGACTTTCCATTGACGTCAATGGGTGGAGTATTTACGGTAAACTGCCCA |
| | CTTGGCAGTAGATCAAGTGTATCATATGCCAAGTACGCCCCCTATTGACGTCAAT |
| | GACGGTAAATGGCCCGCCTGGCATTATGCCCAGTACATGACCTTATGGGACTTTC |
| | CTACTTGGCAGTACATCTACGTATTAGTCATCGCTATTACCATGGTGATGCGGTT |
| | TTGGCAGTACATCAATGGGCGTGGATAGCGGTTTGACTCACGGGGATTTCCAAGT |
| | CTCCACCCCATTGACGTCAATGGGAGTTTGTTTTGGCACCAAAATCAACGGGACT |
| | TTCCAAAATGTCGTAACAACTCCGCCCCATTGACGCAAATGGGCGGTAGGCGTGT |
| | ACGGTGGGAGGTCTATATAAGCAGAGCTCGTTTAGTGAACCGTCAGATCGCCTGG |
| | AGACGCCATCCACGCTGTTTTGACCTCCATAGAAGACACCGGGACCGATCCAGCC |
| | TCCGGACTCTAGAGGATCGAACCCTTGAATTCGCCACCATGGGCTGGTCCTGCAT |
| | CATCCTGTTTCTGGTGGCCACAGCCACAGGCGTGCACTCTGAGGTGCAGCTGGTT |
| | GAATCTGGCGGCGGACTTGTGAAGCCTGGCGGATCTCTGAGACTGAGCTGTGCCG |
| | CCAGCGGCTTCACCTTTAGCAGCTACAGCATGAACTGGGTCCGACAGGCCCCTGG |
| | CAAAGGCCTTGAATGGGTGTCCAGCATCAGCAGCAGCTCCAGCTACATCTACTAC |
| | GCCGACAGCGTGAAGGGCAGATTCACCATCAGCCGGGACAACGCCAAGAACAGCC |
| | TGTACCTGCAGATGAACTCCCTGAGAGCCGAGGACACCGCCGTGTACTATTGTGC |
| | TAGAGGCGCCCCTATTGGAGCCGCCGCTGGATGGTTCGATCCTTGGGGACAGGGA |
| | ACCCTGGTCACCGTTTCTTCTGCCAGCACAAAGGGCCCTAGCGTTTTCCCACTGG |
| | CTCCCAGCAGCAAGAGCACAAGCGGAGGAACAGCTGCCCTGGGCTGTCTGGTCAA |
| | GGACTACTTTCCTGAGCCTGTGACCGTGTCCTGGAACAGCGGAGCACTGACTAGC |
| | GGCGTGCACACATTTCCAGCCGTGCTGCAAAGCAGCGGCCTGTACTCTCTGAGCA |
| | GCGTCGTGACAGTGCCTAGCAGCTCTCTGGGCACCCAGACCTACATCTGCAATGT |
| | GAACCACAAGCCTAGCAACACCAAGGTGGACAAGAAGGTGGAACCCAAGAGCTGC |
| | GACAAGACCCACACCTGTCCTCCATGTCCTGCTCCAGAACTGCTCGGCGGACCTT |
| | CCGTGTTCCTGTTTCCTCCAAAGCCTAAGGACACCCTGATGATCAGCAGAACCCC |
| | TGAAGTGACCTGCGTGGTGGTGGATGTGTCTCACGAGGACCCCGAAGTGAAGTTC |
| | AATTGGTACGTGGACGGCGTGGAAGTGCACAACGCCAAGACCAAGCCTAGAGAGG |
| | AACAGTACAACAGCACCTACAGAGTGGTGTCCGTGCTGACAGTGCTGCACCAGGA |
| | TTGGCTGAACGGCAAAGAGTACAAGTGCAAGGTGTCCAACAAGGCCCTGCCTGCT |
| | CCTATCGAGAAAACCATCAGCAAGGCCAAGGGCCAGCCTCGCGAACCTCAAGTCT |
| | GTACACTGCCTCCTAGCCGGGATGAGCTGACCGAGAATCAGGTGTCCCTGACCTG |
| | TCTCGTGAAGGGCTTCTACCCCTCCGATATCGCCGTGGAATGGGAGAGCAATGGC |
| | CAGCCAGAGAACAACTACAAGACAACCCCTCCTGTGCTGGACAGCGACGGCTCAT |
| | TCTTCCTGTACAGCTGGCTGACCGTGGACAAGTCCAGATGGCAGCAGGGCAACGT |
| | GTTCTCCTGCAGCGTGATGCACGAGGCCCTGCACAACCACTACACCCAGAAGTCC |
| | CTGTCTCTGAGCCCAGGCTAAGGATCCAAGGGTTCGATCCCTACCGGTTAGTAAT |
| | GAGTTTGATATCTCGAGAATCAACCTCTGGATTACAAAATTTGTGAAAGATTGAG |
| | TGGTATTCTTAACTATGTTGCTCCTTTTACGCTATGTGGATACGCTGCTTTAATG |
| | CCTTTGTATCATGCTATTGCTTCCCGTATGGCTTTCATTTTCTCCTCCTTGTATA |
| | AATCCTGGTTGCTGTCTCTTTATGAGGAGTTGTGGCCCGTTGTCAGGCAACGTGG |
| | CGTGGTGTGCACTGTGTTTGCTGACGCAACCCCCACTGGTTGGGGCATTGCCACC |
| | ACCTGTCAGCTCCTTTCCGGGACTTTCGCTTTCCCCCTCCCTATTGCCACGGCGG |
| | AACTCATCGCCGCCTGCCTTGCCCGCTGCTGGACAGGGGCTCGGCTGTTGGGCAC |
| | TGACAATTCCGTGGTGTTGTCGGGGAAGCTGACGTCCTTTCCATGGCTGCTCGCC |
| | TGTGTTGCCACCTGGATTCTGCGCGGGACGTCCTTCTGCTACGTCCCTTCGGCCC |
| | TCAATCCAGCGGACCTTCCTTCCCGCGGCCTGCTGCCGGCTCTGCGGCCTCTTCC |
| | GCGTCTTCGCCTTCGCCCTCAGACGAGTCGGATCTCCCTTTGGGCCGCCTCCCCG |
| | CCTGGAACGGGGGAGGCTAACTGAAACACGGAAGGAGACAATACCGGAAGGAACC |
| | CGCGCTATGACGGCAATAAAAAGACAGAATAAAACGCACGGGTGTTGGGTCGTTT |
| | GTTCATAAACGCGGGGTTCGGTCCCAGGGCTGGCACTCTGTCGATACCCCACCGA |
| | GACCCCATTGGGGCCAATACGCCCGCGTTTCTTCCTTTTCCCCACCCCACCCCCC |
| | AAGTTCGGGTGAAGGCCCAGGGCTCGCAGCCAACGTCGGGGCGGCAGGCCCTGCC |
| | ATAGCAGATCTGCGCAGCTGGGGCTCTAGGGGGTATCCCCACGCGCCCTGTAGCG |
| | GCGCATTAAGCGCGGCGGGTGTGGTGGTTACGCGCAGCGTGACCGCTACACTTGC |
| | CAGCGCCCTAGCGCCCGCTCCTTTCGCTTTCTTCCCTTCCTTTCTCGCCACGTTC |
| | GCCGGCTTTCCCCGTCAAGCTCTAAATCGGGGGCTCCCTTTAGGGTTCCGATTTA |
| | GTGCTTTACGGCACCTCGACCCCAAAAAACTTGATTAGGGTGATGGTTCACGTAG |
| | TGGGCCATCGCCCTGATAGACGGTTTTTCGCCCTTTGACGTTGGAGTCCACGTTC |
| | TTTAATAGTGGACTCTTGTTCCAAACTGGAACAACACTCAACCCTATCTCGGTCT |
| | ATTCTTTTGATTTATAAGGGATTTTGCCGATTTCGGCCTATTGGTTAAAAAATGA |
| | GCTGATTTAACAAAAATTTAACGCGAATTAATTCTGTGGAATGTGTGTCAGTTAG |
| | GGTGTGGAAAGTCCCCAGGCTCCCCAGCAGGCAGAAGTATGCAAAGCATGCATCT |
| | CAATTAGTCAGCAACCAGGTGTGGAAAGTCCCCAGGCTCCCCAGCAGGCAGAAGT |
| | ATGCAAAGCATGCATCTCAATTAGTCAGCAACCATAGTCCCGCCCCTAACTCCGC |
| | CCATCCCGCCCCTAACTCCGCCCAGTTCCGCCCATTCTCCGCCCCATGGCTGACT |
| | AATTTTTTTTATTTATGGAGAGGGGGAGGGGGGGTGTGGGTGTGAGGTATTGGAG |
| | AAGTAGTGAGGAGGCTTTTTTGGAGGCCTAGGCTTTTGCAAAAAGCTCCCGGGAG |
| | CTTGTATATCCATTTTCGGATCTGATCAAGAGACAGGATGAGGATCGTTTCGCAT |
| | GATTGAACAAGATGGATTGCACGCAGGTTCTCCGGCCGCTTGGGTGGAGAGGCTA |
| | TTCGGCTATGACTGGGCACAACAGACAATCGGCTGCTCTGATGCCGCCGTGTTCC |
| | GGCTGTCAGCGCAGGGGCGCCCGGTTCTTTTTGTCAAGACCGACCTGTCCGGTGC |
| | CCTGAATGAACTGCAGGACGAGGCAGCGCGGCTATCGTGGCTGGCCACGACGGGC |
| | GTTCCTTGCGCAGCTGTGCTCGACGTTGTCACTGAAGCGGGAAGGGACTGGCTGC |
| | TATTGGGCGAAGTGCCGGGGCAGGATCTCCTGTCATCTCACCTTGCTCCTGCCGA |
| | GAAAGTATCCATCATGGCTGATGCAATGCGGCGGCTGCATACGCTTGATCCGGCT |
| | ACCTGCCCATTCGACCACCAAGCGAAACATCGCATCGAGCGAGCACGTACTCGGA |
| | TGGAAGCCGGTCTTGTCGATCAGGATGATCTGGACGAAGAGCATCAGGGGCTCGC |
| | GCCAGCCGAACTGTTCGCCAGGCTCAAGGCGCGCATGCCCGACGGCGAGGATCTC |
| | GTCGTGACCCATGGCGATGCCTGCTTGCCGAATATCATGGTGGAAAATGGCCGCT |
| | TTTCTGGATTCATCGACTGTGGCCGGCTGGGTGTGGCGGACCGCTATCAGGACAT |
| | AGCGTTGGCTACCCGTGATATTGCTGAAGAGCTTGGCGGCGAATGGGCTGACCGC |
| | TTCCTCGTGCTTTACGGTATCGCCGCTCCCGATTCGCAGCGCATCGCCTTCTATC |
| | GCCTTCTTGACGAGTTCTTCTGAGCGGGACTCTGGGGTTCGCGAAATGACCGACC |
| | AAGCGACGCCCAACCTGCCATCACGAGATTTCGATTCCACCGCCGCCTTCTATGA |
| | AAGGTTGGGCTTCGGAATCGTTTTCCGGGACGCCGGCTGGATGATCCTCCAGCGC |
| | GGGGATCTCATGCTGGAGTTCTTCGCCCACCCCAACTTGTTTATTGCAGCTTATA |
| | ATGGTTACAAATAAAGCAATAGCATCACAAATTTCACAAATAAAGCATTTTTTTC |
| | ACTGCATTCTAGTTGTGGTTTGTCCAAACTCATCAATGTATCTTATCATGTCTGT |
| | ATACCGTCGACCTCTAGCTAGAGCTTGGCGTAATCATGGTCATAGCTGTTTCCTG |
| | TGTGAAATTGTTATCCGCTCACAATTCCACACAACATACGAGCCGGAAGCATAAA |
| | GTGTAAAGCCTGGGGTGCCTAATGAGTGAGCTAACTCACATTAATTGCGTTGCGC |
| | TCACTGCCCGCTTTCCAGTCGGGAAACCTGTCGTGCCAGCTGCATTAATGAATCG |
| | GCCAACGCGCGGGGAGAGGCGGTTTGCGTATTGGGCGCTCTTCCGCTTCCTCGCT |
| | CACTGACTCGCTGCGCTCGGTCGTTCGGCTGCGGCGAGCGGTATCAGCTCACTCA |
| | AAGGCGGTAATACGGTTATCCACAGAATCAGGGGATAACGCAGGAAAGAACATGT |
| | GAGCAAAAGGCCAGCAAAAGGCCAGGAACCGTAAAAAGGCCGCGTTGCTGGCGTT |
| | TTTCCATAGGCTCCGCCCCCCTGACGAGCATCACAAAAATCGACGCTCAAGTCAG |
| | AGGTGGCGAAACCCGACAGGACTATAAAGATACCAGGCGTTTCCCCCTGGAAGCT |
| | CCCTCGTGCGCTCTCCTGTTCCGACCCTGCCGCTTACCGGATACCTGTCCGCCTT |
| | TCTCCCTTCGGGAAGCGTGGCGCTTTCTCATAGCTCACGCTGTAGGTATCTCAGT |
| | TCGGTGTAGGTCGTTCGCTCCAAGCTGGGCTGTGTGCACGAACCCCCCGTTCAGC |
| | CCGACCGCTGCGCCTTATCCGGTAACTATCGTCTTGAGTCCAACCCGGTAAGACA |
| | CGACTTATCGCCACTGGCAGCAGCCACTGGTAACAGGATTAGCAGAGCGAGGTAT |
| | GTAGGCGGTGCTACAGAGTTCTTGAAGTGGTGGCCTAACTACGGCTACACTAGAA |
| | GAACAGTATTTGGTATCTGCGCTCTGCTGAAGCCAGTTACCTTCGGAAAAAGAGT |
| | TGGTAGCTCTTGATCCGGCAAACAAACCACCGCTGGTAGCGGTGGTTTTTTTGTT |
| | TGCAAGCAGCAGATTACGCGCAGAAAAAAAGGATCTCAAGAAGATCCTTTGATCT |
| | TTTCTACGGGGTCTGACGCTCAGTGGAACGAAAACTCACGTTAAGGGATTTTGGT |
| | CATGAGATTATCAAAAAGGATCTTCACCTAGATCCTTTTAAATTAAAAATGAAGT |
| | TTTAAATCAATCTAAAGTATATATGAGTAAACTTGGTCTGACAGTTACCAATGCT |
| | TAATCAGTGAGGCACCTATCTCAGCGATCTGTCTATTTCGTTCATCCATAGTTGC |
| | CTGACTCCCCGTCGTGTAGATAACTACGATACGGGAGGGCTTACCATCTGGCCCC |
| | AGTGCTGCAATGATACCGCGAGACCCACGCTCACCGGCTCCAGATTTATCAGCAA |
| | TAAACCAGCCAGCCGGAAGGGCCGAGCGCAGAAGTGGTCCTGCAACTTTATCCGC |
| | CTCCATCCAGTCTATTAATTGTTGCCGGGAAGCTAGAGTAAGTAGTTCGCCAGTT |
| | AATAGTTTGCGCAACGTTGTTGCCATTGCTACAGGCATCGTGGTGTCACGCTCGT |
| | CGTTTGGTATGGCTTCATTCAGCTCCGGTTCCCAACGATCAAGGCGAGTTACATG |
| | ATCCCCCATGTTGTGCAAAAAAGCGGTTAGCTCCTTCGGTCCTCCGATCGTTGTC |
| | AGAAGTAAGTTGGCCGCAGTGTTATCACTCATGGTTATGGCAGCACTGCATAATT |
| | CTCTTACTGTCATGCCATCCGTAAGATGCTTTTCTGTGACTGGTGAGTACTCAAC |
| | CAAGTCATTCTGAGAATAGTGTATGCGGCGACCGAGTTGCTCTTGCCCGGCGTCA |
| | ATACGGGATAATACCGCGCCACATAGCAGAACTTTAAAAGTGCTCATCATTGGAA |
| | AACGTTCTTCGGGGCGAAAACTCTCAAGGATCTTACCGCTGTTGAGATCCAGTTC |
| | GATGTAACCCACTCGTGCACCCAACTGATCTTCAGCATCTTTTACTTTCACCAGC |
| | GTTTCTGGGTGAGCAAAAACAGGAAGGCAAAATGCCGCAAAAAAGGGAATAAGGG |
| | CGAGACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAAT |
| | AAACAAATAGGGGTTCCGCGCACATTTCCCCGAAAAGTGCCACCTGACGTCGACG |
| | GATCGGGAGATCTCCCGATCCCCTATGGTCGACTCTCAGTACAATCTGCTCTGAT |
| | GCCGCATAGTTAAGCCAGTATCTGCTCCCTGCTTGTGTGTTGGAGGTCGCTGAGT |
| | AGTGCGCGAGCAAAATTTAAGCTACAACAAGGCAAGGCTTGACCGACAATTGCAT |
| | GAAGAATCTGCTTAGG (SEQ ID NO: 282) |
| AB1310 | GAGGTGCAGCTGGTTGAATCTGGCGGCGGACTTGTGAAGCCTGGCGGATCTCTGA |
| A49MI- | GACTGAGCTGTGCCGCCAGCGGCTTCACCTTTAGCAGCTACAGCATGAACTGGGT |
| VH-CH1- | CCGACAGGCCCCTGGCAAAGGCCTTGAATGGGTGTCCAGCATCAGCAGCAGCTCC |
| Fc | AGCTACATCTACTACGCCGACAGCGTGAAGGGCAGATTCACCATCAGCCGGGACA |
| | ACGCCAAGAACAGCCTGTACCTGCAGATGAACTCCCTGAGAGCCGAGGACACCGC |
| | CGTGTACTATTGTGCTAGAGGCGCCCCTATTGGAGCCGCCGCTGGATGGTTCGAT |
| | CCTTGGGGACAGGGAACCCTGGTCACCGTTTCTTCTGCCAGCACAAAGGGCCCTA |
| | GCGTTTTCCCACTGGCTCCCAGCAGCAAGAGCACAAGCGGAGGAACAGCTGCCCT |
| | GGGCTGTCTGGTCAAGGACTACTTTCCTGAGCCTGTGACCGTGTCCTGGAACAGC |
| | GGAGCACTGACTAGCGGCGTGCACACATTTCCAGCCGTGCTGCAAAGCAGCGGCC |
| | TGTACTCTCTGAGCAGCGTCGTGACAGTGCCTAGCAGCTCTCTGGGCACCCAGAC |
| | CTACATCTGCAATGTGAACCACAAGCCTAGCAACACCAAGGTGGACAAGAAGGTG |
| | GAACCCAAGAGCTGCGACAAGACCCACACCTGTCCTCCATGTCCTGCTCCAGAAC |
| | TGCTCGGCGGACCTTCCGTGTTCCTGTTTCCTCCAAAGCCTAAGGACACCCTGAT |
| | GATCAGCAGAACCCCTGAAGTGACCTGCGTGGTGGTGGATGTGTCTCACGAGGAC |
| | CCCGAAGTGAAGTTCAATTGGTACGTGGACGGCGTGGAAGTGCACAACGCCAAGA |
| | CCAAGCCTAGAGAGGAACAGTACAACAGCACCTACAGAGTGGTGTCCGTGCTGAC |
| | AGTGCTGCACCAGGATTGGCTGAACGGCAAAGAGTACAAGTGCAAGGTGTCCAAC |
| | AAGGCCCTGCCTGCTCCTATCGAGAAAACCATCAGCAAGGCCAAGGGCCAGCCTC |
| | GCGAACCTCAAGTCTGTACACTGCCTCCTAGCCGGGATGAGCTGACCGAGAATCA |
| | GGTGTCCCTGACCTGTCTCGTGAAGGGCTTCTACCCCTCCGATATCGCCGTGGAA |
| | TGGGAGAGCAATGGCCAGCCAGAGAACAACTACAAGACAACCCCTCCTGTGCTGG |
| | ACAGCGACGGCTCATTCTTCCTGTACAGCTGGCTGACCGTGGACAAGTCCAGATG |
| | GCAGCAGGGCAACGTGTTCTCCTGCAGCGTGATGCACGAGGCCCTGCACAACCAC |
| | TACACCCAGAAGTCCCTGTCTCTGAGCCCAGGC (SEQ ID NO: 546) |
| AB1310 | CTCTCCTGTTCCGACCCTGCCGCTTACCGGATACCTGTCCGCCTTTCTCCCTTCG |
| A49MI- | GGAAGCGTGGCGCTTTCTCATAGCTCACGCTGTAGGTATCTCAGTTCGGTGTAGG |
| VL-CL | TCGTTCGCTCCAAGCTGGGCTGTGTGCACGAACCCCCCGTTCAGCCCGACCGCTG |
| | CGCCTTATCCGGTAACTATCGTCTTGAGTCCAACCCGGTAAGACACGACTTATCG |
| | CCACTGGCAGCAGCCACTGGTAACAGGATTAGCAGAGCGAGGTATGTAGGCGGTG |
| | CTACAGAGTTCTTGAAGTGGTGGCCTAACTACGGCTACACTAGAAGAACAGTATT |
| | TGGTATCTGCGCTCTGCTGAAGCCAGTTACCTTCGGAAAAAGAGTTGGTAGCTCT |
| | TGATCCGGCAAACAAACCACCGCTGGTAGCGGTGGTTTTTTTGTTTGCAAGCAGC |
| | AGATTACGCGCAGAAAAAAAGGATCTCAAGAAGATCCTTTGATCTTTTCTACGGG |
| | GTCTGACGCTCAGTGGAACGAAAACTCACGTTAAGGGATTTTGGTCATGAGATTA |
| | TCAAAAAGGATCTTCACCTAGATCCTTTTAAATTAAAAATGAAGTTTTAAATCAA |
| | TCTAAAGTATATATGAGTAAACTTGGTCTGAGAGTTACCAATGCTTAATGAGTGA |
| | GGCACCTATCTCAGCGATCTGTCTATTTCGTTCATCCATAGTTGCCTGACTCCCC |
| | GTCGTGTAGATAACTACGATACGGGAGGGCTTACCATCTGGCCCCAGTGCTGCAA |
| | TGATACCGCGAGACCCACGCTCACCGGCTCCAGATTTATCAGCAATAAACCAGCC |
| | AGCCGGAAGGGCCGAGCGCAGAAGTGGTCCTGCAACTTTATCCGCCTCCATCCAG |
| | TCTATTAATTGTTGCCGGGAAGCTAGAGTAAGTAGTTCGCCAGTTAATAGTTTGC |
| | GCAACGTTGTTGCCATTGCTACAGGCATCGTGGTGTCACGCTCGTCGTTTGGTAT |
| | GGCTTCATTCAGCTCCGGTTCCCAACGATCAAGGCGAGTTACATGATCCCCCATG |
| | TTGTGCAAAAAAGCGGTTAGCTCCTTCGGTCCTCCGATCGTTGTCAGAAGTAAGT |
| | TGGCCGCAGTGTTATCACTCATGGTTATGGCAGCACTGCATAATTCTCTTACTGT |
| | CATGCCATCCGTAAGATGCTTTTCTGTGACTGGTGAGTACTCAACCAAGTCATTC |
| | TGAGAATAGTGTATGCGGCGACCGAGTTGCTCTTGCCCGGCGTCAATACGGGATA |
| | ATACCGCGCCACATAGCAGAACTTTAAAAGTGCTCATCATTGGAAAACGTTCTTC |
| | GGGGCGAAAACTCTCAAGGATCTTACCGCTGTTGAGATCCAGTTCGATGTAACCC |
| | ACTCGTGCACCCAACTGATCTTCAGCATCTTTTACTTTCACCAGCGTTTCTGGGT |
| | GAGCAAAAACAGGAAGGCAAAATGCCGCAAAAAAGGGAATAAGGGCGACACGGAA |
| | ATGTTGAATACTCATACTCTTCCTTTTTCAATATTATTGAAGCATTTATCAGGGT |
| | TATTGTCTCATGAGCGGATACATATTTGAATGTATTTAGAAAAATAAACAAATAG |
| | GGGTTCCGCGCACATTTCCCCGAAAAGTGCCACCTGACGTCGACGGATCGGGAGA |
| | TCTCCCGATCCCCTATGGTCGACTCTCAGTACAATCTGCTCTGATGCCGCATAGT |
| | TAAGCCAGTATCTGCTCCCTGCTTGTGTGTTGGAGGTCGCTGAGTAGTGCGCGAG |
| | CAAAATTTAAGCTACAACAAGGCAAGGCTTGACCGACAATTGCATGAAGAATCTG |
| | CTTAGGGTTAGGCGTTTTGCGCTGCTTCGCGATGTACGGGCCAGATATACGCGTT |
| | GAGATTGATTATTGAGTAGTTATTAATAGTAATCAATTACGGGGTCATTAGTTCA |
| | TAGCCCATATATGGAGTTCCGCGTTACATAACTTACGGTAAATGGCCCGCCTGGC |
| | TGACCGCCCAACGACCCCCGCCCATTGACGTCAATAATGACGTATGTTCCCATAG |
| | TAACGCCAATAGGGACTTTCCATTGACGTCAATGGGTGGAGTATTTACGGTAAAC |
| | TGCCCACTTGGCAGTACATCAAGTGTATCATATGCCAAGTACGCCCCCTATTGAC |
| | GTCAATGACGGTAAATGGCCCGCCTGGCATTATGCCCAGTACATGACCTTATGGG |
| | ACTTTCCTACTTGGCAGTAGATCTACGTATTAGTCATCGCTATTACCATGGTGAT |
| | GCGGTTTTGGCAGTACATCAATGGGCGTGGATAGCGGTTTGACTCACGGGGATTT |
| | CCAAGTCTCCACCCCATTGACGTCAATGGGAGTTTGTTTTGGCACCAAAATCAAC |
| | GGGACTTTCCAAAATGTCGTAACAACTCCGCCCCATTGACGCAAATGGGCGGTAG |
| | GCGTGTACGGTGGGAGGTCTATATAAGCAGAGCTCGTTTAGTGAACCGTCAGATC |
| | GCCTGGAGACGCCATCCACGCTGTTTTGACCTCCATAGAAGACACCGGGACCGAT |
| | CCAGCCTCCGGACTCTAGAGGATCGAACCCTTGCCACCATGGACATGAGAGTTCC |
| | AGCTCAGCTGCTGGGCCTGCTGCTGCTTTGGCTTCCTGGCGCTAGATGCGACATC |
| | CAGATGACACAGAGCCCCAGCTCCGTGTCTGCCTCTGTGGGAGACAGAGTGACCA |
| | TCACCTGTAGAGCCAGCCAGGGCATCTCTTCTTGGCTGGCCTGGTATCAGCAGAA |
| | GCCTGGCAAGGCCCCTAAGCTGCTGATCTATGCCGCTAGCTCTCTGCAGTCTGGC |
| | GTGCCCTCTAGATTTTCTGGCAGCGGCTCTGGCACCGACTTCACCCTGACCATAT |
| | CTAGCCTGCAGCCTGAGGACTTCGCCACCTACTATTGTCAGCAGGGCGTGTCCTT |
| | TCCACGGACCTTTGGCGGCGGAACAAAGGTGGAAATCAAGCGGACAGTGGCCGCT |
| | CCTAGCGTGTTCATCTTTCCACCTAGCGACGAGCAGCTGAAGTCCGGCACAGCCT |
| | CTGTTGTGTGCCTGCTGAACAACTTCTACCCCAGAGAAGCCAAGGTGCAGTGGAA |
| | GGTGGACAATGCCCTGCAGAGCGGCAACAGCCAAGAGAGCGTGACAGAGCAGGAC |
| | AGCAAGGACTCCACCTACAGCCTGAGCAGCACCCTGACACTGAGCAAGGCCGACT |
| | ACGAGAAGCACAAAGTGTACGCCTGCGAAGTGACCCACCAGGGCCTTTCTAGCCC |
| | TGTGACCAAGAGCTTCAACCGGGGCGAGTGTTGAAAGGGTTCGATCCCTACCGGT |
| | TAGTAATGAGTTTGATATCTCGACAATCAACCTCTGGATTACAAAATTTGTGAAA |
| | GATTGACTGGTATTCTTAACTATGTTGCTCCTTTTACGCTATGTGGATACGCTGC |
| | TTTAATGCCTTTGTATCATGCTATTGCTTCCCGTATGGCTTTCATTTTCTCCTCC |
| | TTGTATAAATCCTGGTTGCTGTCTCTTTATGAGGAGTTGTGGCCCGTTGTCAGGC |
| | AACGTGGCGTGGTGTGCACTGTGTTTGCTGACGCAACCCCCACTGGTTGGGGCAT |
| | TGCCACCACCTGTCAGCTCCTTTCCGGGACTTTCGCTTTCCCCCTCCCTATTGCC |
| | ACGGCGGAACTCATCGCCGCCTGCCTTGCCCGCTGCTGGACAGGGGCTCGGCTGT |
| | TGGGCACTGACAATTCCGTGGTGTTGTCGGGGAAGCTGACGTCCTTTCCATGGCT |
| | GCTCGCCTGTGTTGCCACCTGGATTCTGCGCGGGACGTCCTTCTGCTACGTCCCT |
| | TCGGCCCTCAATCCAGCGGACCTTCCTTCCCGCGGCCTGCTGCCGGCTCTGCGGC |
| | CTCTTCCGCGTCTTCGCCTTCGCCCTCAGACGAGTCGGATCTCCCTTTGGGCCGC |
| | CTCCCCGCCTGGAACGGGGGAGGCTAACTGAAACACGGAAGGAGACAATACCGGA |
| | AGGAACCCGCGCTATGACGGCAATAAAAAGACAGAATAAAACGCACGGGTGTTGG |
| | GTCGTTTGTTCATAAACGCGGGGTTCGGTCCCAGGGCTGGCACTCTGTCGATACC |
| | CCACCGAGACCCCATTGGGGCCAATACGCCCGCGTTTCTTCCTTTTCCCCACCCC |
| | ACCCCCCAAGTTCGGGTGAAGGCCCAGGGCTCGCAGCCAACGTCGGGGCGGCAGG |
| | CCCTGCCATAGCAGATCTGCGCAGCTGGGGCTCTAGGGGGTATCCCCACGCGCCC |
| | TGTAGCGGCGCATTAAGCGCGGCGGGTGTGGTGGTTACGCGCAGCGTGACCGCTA |
| | CACTTGCCAGCGCCCTAGCGCCCGCTCCTTTCGCTTTCTTCCCTTCCTTTCTCGC |
| | CACGTTCGCCGGCTTTCCCCGTCAAGCTCTAAATCGGGGGCTCCCTTTAGGGTTC |
| | CGATTTAGTGCTTTACGGCACCTCGACCCCAAAAAACTTGATTAGGGTGATGGTT |
| | CACGTAGTGGGCCATCGCCCTGATAGACGGTTTTTCGCCCTTTGACGTTGGAGTC |
| | CACGTTCTTTAATAGTGGACTCTTGTTCCAAACTGGAACAACACTCAACCCTATC |
| | TCGGTCTATTCTTTTGATTTATAAGGGATTTTGCCGATTTCGGCCTATTGGTTAA |
| | AAAATGAGCTGATTTAACAAAAATTTAACGCGAATTAATTCTGTGGAATGTGTGT |
| | CAGTTAGGGTGTGGAAAGTCCCCAGGCTCCCCAGCAGGCAGAAGTATGCAAAGCA |
| | TGCATCTCAATTAGTCAGCAACCAGGTGTGGAAAGTCCCCAGGCTCCCCAGCAGG |
| | CAGAAGTATGCAAAGCATGCATCTCAATTAGTCAGCAACCATAGTCCCGCCCCTA |
| | ACTCCGCCCATCCCGCCCCTAACTCCGCCCAGTTCCGCCCATTCTCCGCCCCATG |
| | GCTGACTAATTTTTTTTATTTATGCAGAGGCCGAGGCCGCCTCTGCCTCTGAGCT |
| | ATTCCAGAAGTAGTGAGGAGGCTTTTTTGGAGGCCTAGGCTTTTGCAAAAAGCTC |
| | CCGGGAGCTTGTATATCCATTTTCGGATCTGATCAAGAGACAGGATGAGGATCGT |
| | TTCGCATGATTGAACAAGATGGATTGCACGCAGGTTCTCCGGCCGCTTGGGTGGA |
| | GAGGCTATTCGGCTATGACTGGGCACAACAGACAATCGGCTGCTCTGATGCCGCC |
| | GTGTTCCGGCTGTCAGCGCAGGGGCGCCCGGTTCTTTTTGTCAAGACCGACCTGT |
| | CCGGTGCCCTGAATGAACTGCAGGACGAGGCAGCGCGGCTATCGTGGCTGGCCAC |
| | GACGGGCGTTCCTTGCGCAGCTGTGCTCGACGTTGTCACTGAAGCGGGAAGGGAC |
| | TGGCTGCTATTGGGCGAAGTGCCGGGGCAGGATCTCCTGTCATCTCACCTTGCTC |
| | CTGCCGAGAAAGTATCCATCATGGCTGATGCAATGCGGCGGCTGCATACGCTTGA |
| | TCCGGCTACCTGCCCATTCGACCACCAAGCGAAACATCGCATCGAGCGAGCACGT |
| | ACTCGGATGGAAGCCGGTCTTGTCGATCAGGATGATCTGGACGAAGAGCATCAGG |
| | GGCTCGCGCCAGCCGAACTGTTCGCCAGGCTCAAGGCGCGCATGCCCGACGGCGA |
| | GGATCTCGTCGTGACCCATGGCGATGCCTGCTTGCCGAATATCATGGTGGAAAAT |
| | GGCCGCTTTTCTGGATTCATCGACTGTGGCCGGCTGGGTGTGGCGGACCGCTATC |
| | AGGACATAGCGTTGGCTACCCGTGATATTGCTGAAGAGCTTGGCGGCGAATGGGC |
| | TGACCGCTTCCTCGTGCTTTACGGTATCGCCGCTCCCGATTCGCAGCGCATCGCC |
| | TTCTATCGCCTTCTTGACGAGTTCTTCTGAGCGGGACTCTGGGGTTCGCGAAATG |
| | ACCGACCAAGCGACGCCCAACCTGCCATCACGAGATTTCGATTCCACCGCCGCCT |
| | TCTATGAAAGGTTGGGCTTCGGAATCGTTTTCCGGGACGCCGGCTGGATGATCCT |
| | CCAGCGCGGGGATCTCATGCTGGAGTTCTTCGCCCACCCCAACTTGTTTATTGCA |
| | GCTTATAATGGTTACAAATAAAGCAATAGCATCACAAATTTCACAAATAAAGCAT |
| | TTTTTTCACTGCATTCTAGTTGTGGTTTGTCCAAACTCATCAATGTATCTTATCA |
| | TGTCTGTATACCGTCGACCTCTAGCTAGAGCTTGGCGTAATCATGGTCATAGCTG |
| | TTTCCTGTGTGAAATTGTTATCCGCTCACAATTCCACACAACATACGAGCCGGAA |
| | GCATAAAGTGTAAAGCCTGGGGTGCCTAATGAGTGAGCTAACTCACATTAATTGC |
| | GTTGCGCTCACTGCCCGCTTTCCAGTCGGGAAACCTGTCGTGCCAGCTGCATTAA |
| | TGAATCGGCCAACGCGCGGGGAGAGGCGGTTTGCGTATTGGGCGCTCTTCCGCTT |
| | CCTCGCTCACTGACTCGCTGCGCTCGGTCGTTCGGCTGCGGCGAGCGGTATCAGC |
| | TCACTCAAAGGCGGTAATACGGTTATCCACAGAATCAGGGGATAACGCAGGAAAG |
| | AACATGTGAGCAAAAGGCCAGCAAAAGGCCAGGAACCGTAAAAAGGCCGCGTTGC |
| | TGGCGTTTTTCCATAGGCTCCGCCCCCCTGACGAGCATCACAAAAATCGACGCTC |
| | AAGTCAGAGGTGGCGAAACCCGACAGGACTATAAAGATACCAGGCGTTTCCCCCT |
| | GGAAGCTCCCTCGTGCG (SEQ ID NO: 283) |
| AB1310 | GACATCCAGATGACACAGAGCCCCAGCTCCGTGTCTGCCTCTGTGGGAGACAGAG |
| A49MI- | TGACCATCACCTGTAGAGCCAGCCAGGGCATCTCTTCTTGGCTGGCCTGGTATCA |
| VL-CL | GCAGAAGCCTGGCAAGGCCCCTAAGCTGCTGATCTATGCCGCTAGCTCTCTGCAG |
| | TCTGGCGTGCCCTCTAGATTTTCTGGCAGCGGCTCTGGCACCGACTTCACCCTGA |
| | CCATATCTAGCCTGCAGCCTGAGGACTTCGCCACCTACTATTGTCAGCAGGGCGT |
| | GTCCTTTCCACGGACCTTTGGCGGCGGAACAAAGGTGGAAATCAAGCGGACAGTG |
| | GCCGCTCCTAGCGTGTTCATCTTTCCACCTAGCGACGAGCAGCTGAAGTCCGGCA |
| | CAGCCTCTGTTGTGTGCCTGCTGAACAACTTCTACCCCAGAGAAGCCAAGGTGCA |
| | GTGGAAGGTGGACAATGCCCTGCAGAGCGGCAACAGCCAAGAGAGCGTGACAGAG |
| | CAGGACAGCAAGGACTCCACCTACAGCCTGAGCAGCACCCTGACACTGAGCAAGG |
| | CCGACTACGAGAAGCACAAAGTGTACGCCTGCGAAGTGACCCACCAGGGCCTTTC |
| | TAGCCCTGTGACCAAGAGCTTCAACCGGGGCGAGTGT (SEQ ID NO: 547) |
| AB1783 | AAGCTTACCGCCACCATGGACATGCGCGTCCCCGCGCAGCTCCTCGGCCTCCTGC |
| 5T4-scFv | TGCTCTGGCTCCCCGGCGCCCGCTGCGACATCCAACTGACCCAGTCCCCGTCCTC |
| (scFv- | CCTGAGCGCATCTGTCGGCGACCGGGTGACCATCACCTGCCGGGCCTCGCAGTCC |
| AB1002- | GTGACCACGTCGAACTACAACTACATGCACTGGTTCCAGCAGAAGCCCGGTAAGG |
| VL-VH- | CGCCCAAGCTGCTCATCAAGTTCGCGTCCAACCTGGAGTCGGGCGTGCCGTCGCG |
| Fc) | GTTCTCCGGCTCCGGCAGCGGCACCGACTTCACGCTGACGATCTCGTCCCTCCAG |
| | CCCGAGGACTTCGCCACGTACTACTGCCAGCACTCCTGGGAGATCCCGTGGACGT |
| | TCGGGTGCGGCACCAAGGTCGAGATCAAGGGAGGCGGGGGCAGTGGGGGCGGAGG |
| | CTCTGGGGGCGGCGGCAGTGGCGGCGGCGGCTCGGAGGTCCAGCTGGTAGAGTCC |
| | GGAGGCGGCCTCGTGAAGCCGGGCGGCTCCCTGCGCCTGTCCTGCGCGGCCAGCC |
| | GTTTCACCTTCTCGGACTTCGGGATGCATTGGGTGCGCCAGGCCCCAGGCAAGTG |
| | CCTGGAGTGGGTTTCCTACATCTCGTCCGGCTCGAGTACCATCTATTACGCCGAC |
| | AGCGTCAAGGGACGCTTCACGATCTCGCGGGACAACGCCAAGAACAGCCTTTACC |
| | TGCAGATGAACAGCCTGCGCGCGGAGGACACCGCCGTCTACTACTGCGCCTCGTC |
| | CCAGAGCTATTACCGGGGTACCCTCGACTACTGGGGCCAGGGGACGACGGTGACC |
| | GTCAGCTCCGCCAGCGACAAGACCCACACGTGCCCCCCGTGCCCGGCCCCGGAGC |
| | TGCTGGGAGGCCCGTCCGTGTTCCTGTTCCCCCCGAAGCCCAAGGACACCCTGAT |
| | GATCTCGCGCACCCCGGAGGTGACCTGCGTAGTCGTCGACGTGTCCCACGAGGAC |
| | CCCGAGGTGAAGTTCAACTGGTACGTGGACGGCGTCGAGGTCCACAACGCGAAGA |
| | CCAAGCCGCGCGAGGAGCAGTACAACAGCACCTACCGGGTCGTGAGCGTCCTGAC |
| | CGTGCTGCACCAAGACTGGCTCAACGGTAAGGAGTACAAGTGTAAGGTGAGCAAC |
| | AAGGCCCTCCCTGCCCCGATTGAGAAGACCATCTCGAAGGCTAAGGGCCAGCCGC |
| | GGGAGCCCCGGGTGTACACGCTCCCGCCCTGCCGGGACGAACTGACCAAAAACCA |
| | GGTGTCCCTGACGTGTCTGGTGAAGGGCTTCTACCCCTCCGACATCGCCGTCGAG |
| | TGGGAGTCCAATGGTCAGCCCGAGAACAACTACAAGACGACCCCGCCCGTGCTGG |
| | TGAGCGACGGGTCCTTCACCCTGTATTCCAAGCTCACGGTCGACAAGTCCCGCTG |
| | GCAGCAGGGCAACGTCTTCTCGTGCTCCGTGATGCACGAGGCCCTCCACAACCAC |
| | TACACCCAGAAGTCACTGTCACTCTCCCCCGGCTGATAATCTAGA (SEQ ID |
| | NO: 281) |
| AB1783 | GACATCCAACTGACCCAGTCCCCGTCCTCCCTGAGCGCATCTGTCGGCGACCGGG |
| 5T4-scFv | TGACCATCACCTGCCGGGCCTCGCAGTCCGTGACCACGTCGAACTACAACTACAT |
| (scFv- | GCACTGGTTCCAGCAGAAGCCCGGTAAGGCGCCCAAGCTGCTCATCAAGTTCGCG |
| AB1002- | TCCAACCTGGAGTCGGGCGTGCCGTCGCGGTTCTCCGGCTCCGGCAGCGGCACCG |
| VL-VH- | ACTTCACGCTGACGATCTCGTCCCTCCAGCCCGAGGACTTCGCCACGTACTACTG |
| Fc) | CCAGCACTCCTGGGAGATCCCGTGGACGTTCGGGTGCGGCACCAAGGTCGAGATC |
| | AAGGGAGGCGGGGGCAGTGGGGGCGGAGGCTCTGGGGGCGGCGGCAGTGGCGGCG |
| | GCGGCTCGGAGGTCCAGCTGGTAGAGTCCGGAGGCGGCCTCGTGAAGCCGGGCGG |
| | CTCCCTGCGCCTGTCCTGCGCGGCCAGCCGTTTCACCTTCTCGGACTTCGGGATG |
| | CATTGGGTGCGCCAGGCCCCAGGCAAGTGCCTGGAGTGGGTTTCCTACATCTCGT |
| | CCGGCTCGAGTACCATCTATTACGCCGACAGCGTCAAGGGACGCTTCACGATCTC |
| | GCGGGACAACGCCAAGAACAGCCTTTACCTGCAGATGAACAGCCTGCGCGCGGAG |
| | GACACCGCCGTCTACTACTGCGCCTCGTCCCAGAGCTATTACCGGGGTACCCTCG |
| | ACTACTGGGGCCAGGGGACGACGGTGACCGTCAGCTCCGCCAGCGACAAGACCCA |
| | CACGTGCCCCCCGTGCCCGGCCCCGGAGCTGCTGGGAGGCCCGTCCGTGTTCCTG |
| | TTCCCCCCGAAGCCCAAGGACACCCTGATGATCTCGCGCACCCCGGAGGTGACCT |
| | GCGTAGTCGTCGACGTGTCCCACGAGGACCCCGAGGTGAAGTTCAACTGGTACGT |
| | GGACGGCGTCGAGGTCCACAACGCGAAGACCAAGCCGCGCGAGGAGCAGTACAAC |
| | AGCACCTACCGGGTCGTGAGCGTCCTGACCGTGCTGCACCAAGACTGGCTCAACG |
| | GTAAGGAGTACAAGTGTAAGGTGAGCAACAAGGCCCTCCCTGCCCCGATTGAGAA |
| | GACCATCTCGAAGGCTAAGGGCCAGCCGCGGGAGCCCCGGGTGTACACGCTCCCG |
| | CCCTGCCGGGACGAACTGACCAAAAACCAGGTGTCCCTGACGTGTCTGGTGAAGG |
| | GCTTCTACCCCTCCGACATCGCCGTCGAGTGGGAGTCCAATGGTCAGCCCGAGAA |
| | CAACTACAAGACGACCCCGCCCGTGCTGGTGAGCGACGGGTCCTTCACCCTGTAT |
| | TCCAAGCTCACGGTCGACAAGTCCCGCTGGCAGCAGGGCAACGTCTTCTCGTGCT |
| | CCGTGATGCACGAGGCCCTCCACAACCACTACACCCAGAAGTCACTGTCACTCTC |
| | CCCCGGC (SEQ ID NO: 548) |
| AB1783 | GTTAGGCGTTTTGCGCTGCTTCGCGATGTACGGGCCAGATATACGCGTTGACATT |
| A49MI- | GATTATTGAGTAGTTATTAATAGTAATCAATTACGGGGTCATTAGTTCATAGCCC |
| VH-CH1- | ATATATGGAGTTCCGCGTTACATAACTTACGGTAAATGGCCCGCCTGGCTGACCG |
| Fc | CCCAACGACCCCCGCCCATTGACGTCAATAATGACGTATGTTCCCATAGTAACGC |
| | CAATAGGGACTTTCCATTGACGTCAATGGGTGGAGTATTTACGGTAAACTGCCCA |
| | CTTGGGAGTAGATCAAGTGTATCATATGCCAAGTACGCCCCCTATTGAGGTCAAT |
| | GACGGTAAATGGCCCGCCTGGCATTATGCCCAGTACATGACCTTATGGGACTTTC |
| | CTACTTGGCAGTACATCTACGTATTAGTCATCGCTATTACCATGGTGATGCGGTT |
| | TTGGCAGTACATCAATGGGCGTGGATAGCGGTTTGACTCACGGGGATTTCCAAGT |
| | CTCCACCCCATTGACGTCAATGGGAGTTTGTTTTGGCACCAAAATCAACGGGACT |
| | TTCCAAAATGTCGTAACAACTCCGCCCCATTGACGCAAATGGGCGGTAGGCGTGT |
| | ACGGTGGGAGGTCTATATAAGCAGAGCTCGTTTAGTGAACCGTCAGATCGCCTGG |
| | AGACGCCATCCACGCTGTTTTGACCTCCATAGAAGACACCGGGACCGATCCAGCC |
| | TCCGGACTCTAGAGGATCGAACCCTTAAGCTTACCGCCACCATGGACATGAGAGT |
| | GCCCGCTCAGCTGCTGGGACTGCTGTTGTTGTGGTTGCCTGGCGCTAGATGCGAG |
| | GTGCAGCTGGTTGAATCTGGCGGCGGACTTGTGAAGCCTGGCGGATCTCTGAGAC |
| | TGTCTTGTGCCGCCTCCGGCTTCACCTTCTCCAGCTACTCTATGAACTGGGTCCG |
| | ACAGGCCCCTGGCAAAGGACTGGAATGGGTGTCCTCTATCTCCTCCAGCTCCTCC |
| | TACATCTACTACGCCGACTCCGTGAAGGGCAGATTCACCATCAGCCGGGACAACG |
| | CCAAGAACTCCCTGTACCTGCAGATGAACAGCCTGAGAGCCGAGGACACCGCCGT |
| | GTACTATTGTGCTAGAGGCGCTCCTATCGGCGCTGCCGCTGGATGGTTCGATCCT |
| | TGGGGACAGGGAACCCTGGTCACCGTGTCCTCTGCTTCTACCAAGGGACCCAGCG |
| | TGTTCCCTCTGGCTCCTTCCAGCAAGTCTACCTCTGGCGGAACAGCTGCTCTGGG |
| | CTGCCTGGTCAAGGACTACTTTCCTGAGCCTGTGACCGTGTCTTGGAACTCTGGC |
| | GCTCTGACATCCGGCGTGCACACATTTCCAGCTGTGCTGCAGTCCTCCGGCCTGT |
| | ACTCTCTGTCCTCTGTCGTGACCGTGCCTTCCAGCTCTCTGGGAACCCAGACCTA |
| | CATCTGCAATGTGAACCACAAGCCTTCCAACACCAAGGTGGACAAGAAGGTGGAA |
| | CCCAAGTCCTGCGACAAGACCCACACCTGTCCTCCATGTCCTGCTCCAGAACTGC |
| | TCGGCGGACCTTCCGTGTTCCTGTTTCCTCCAAAGCCTAAGGACACCCTGATGAT |
| | CTCTCGGACCCCTGAAGTGACCTGCGTGGTGGTGGATGTGTCTCACGAGGATCCC |
| | GAAGTGAAGTTCAATTGGTACGTGGACGGCGTGGAAGTGCACAATGCCAAGACCA |
| | AGCCTAGAGAGGAACAGTACAACTCCACCTACAGAGTGGTGTCCGTGCTGACCGT |
| | GCTGCACCAGGATTGGCTGAACGGCAAAGAGTACAAGTGCAAGGTGTCCAACAAG |
| | GCCCTGCCTGCTCCAATCGAAAAGACCATCTCCAAGGCCAAGGGCCAGCCTCGGG |
| | AACCTCAAGTCTGTACCTTGCCTCCTAGCCGGGATGAGCTGACCGAGAATCAGGT |
| | GTCCCTGACCTGCCTCGTGAAGGGATTCTACCCCTCCGATATCGCCGTGGAATGG |
| | GAGTCTAATGGCCAGCCAGAGAACAACTACAAGACAACCCCTCCTGTGCTGGACT |
| | CCGACGGCTCCTTCTTTCTGTACTCCTGGCTGACAGTGGACAAGTCCAGATGGCA |
| | GCAGGGCAACGTGTTCTCCTGCTCCGTGATGCACGAGGCCCTGCACAATCACTAC |
| | ACCCAGAAGTCCCTGTCTCTGTCCCCTGGCTGATGATCTAGAAAGGGTTCGATCC |
| | CTACCGGTTAGTAATGAGTTTGATATCTCGAGAATCAACCTCTGGATTACAAAAT |
| | TTGTGAAAGATTGACTGGTATTCTTAACTATGTTGCTCCTTTTACGCTATGTGGA |
| | TACGCTGCTTTAATGCCTTTGTATCATGCTATTGCTTCCCGTATGGCTTTCATTT |
| | TCTCCTCCTTGTATAAATCCTGGTTGCTGTCTCTTTATGAGGAGTTGTGGCCCGT |
| | TGTCAGGCAACGTGGCGTGGTGTGCACTGTGTTTGCTGACGCAACCCCCACTGGT |
| | TGGGGCATTGCCACCACCTGTCAGCTCCTTTCCGGGACTTTCGCTTTCCCCCTCC |
| | CTATTGCCACGGCGGAACTCATCGCCGCCTGCCTTGCCCGCTGCTGGACAGGGGC |
| | TCGGCTGTTGGGCACTGACAATTCCGTGGTGTTGTCGGGGAAGCTGACGTCCTTT |
| | CCATGGCTGCTCGCCTGTGTTGCCACCTGGATTCTGCGCGGGACGTCCTTCTGCT |
| | ACGTCCCTTCGGCCCTCAATCCAGCGGACCTTCCTTCCCGCGGCCTGCTGCCGGC |
| | TCTGCGGCCTCTTCCGCGTCTTCGCCTTCGCCCTCAGACGAGTCGGATCTCCCTT |
| | TGGGCCGCCTCCCCGCCTGGAACGGGGGAGGCTAACTGAAACACGGAAGGAGACA |
| | ATACCGGAAGGAACCCGCGCTATGACGGCAATAAAAAGACAGAATAAAACGCACG |
| | GGTGTTGGGTCGTTTGTTCATAAACGCGGGGTTCGGTCCCAGGGCTGGCACTCTG |
| | TCGATACCCCACCGAGACCCCATTGGGGCCAATACGCCCGCGTTTCTTCCTTTTC |
| | CCCACCCCACCCCCCAAGTTCGGGTGAAGGCCCAGGGCTCGCAGCCAACGTCGGG |
| | GCGGCAGGCCCTGCCATAGCAGATCTGCGCAGCTGGGGCTCTAGGGGGTATCCCC |
| | ACGCGCCCTGTAGCGGCGCATTAAGCGCGGCGGGTGTGGTGGTTACGCGCAGCGT |
| | GACCGCTACACTTGCCAGCGCCCTAGCGCCCGCTCCTTTCGCTTTCTTCCCTTCC |
| | TTTCTCGCCACGTTCGCCGGCTTTCCCCGTCAAGCTCTAAATCGGGGGCTCCCTT |
| | TAGGGTTCCGATTTAGTGCTTTACGGCACCTCGACCCCAAAAAACTTGATTAGGG |
| | TGATGGTTCACGTAGTGGGCCATCGCCCTGATAGACGGTTTTTCGCCCTTTGACG |
| | TTGGAGTCCACGTTCTTTAATAGTGGACTCTTGTTCCAAACTGGAACAACACTCA |
| | ACCCTATCTCGGTCTATTCTTTTGATTTATAAGGGATTTTGCCGATTTCGGCCTA |
| | TTGGTTAAAAAATGAGCTGATTTAACAAAAATTTAACGCGAATTAATTCTGTGGA |
| | ATGTGTGTCAGTTAGGGTGTGGAAAGTCCCCAGGCTCCCCAGCAGGCAGAAGTAT |
| | GCAAAGCATGCATCTCAATTAGTCAGCAACCAGGTGTGGAAAGTCCCCAGGCTCC |
| | CCAGCAGGCAGAAGTATGCAAAGCATGCATCTCAATTAGTCAGCAACCATAGTCC |
| | CGCCCCTAACTCCGCCCATCCCGCCCCTAACTCCGCCCAGTTCCGCCCATTCTCC |
| | GCCCCATGGCTGACTAATTTTTTTTATTTATGCAGAGGCCGAGGCCGCCTCTGCC |
| | TCTGAGCTATTCCAGAAGTAGTGAGGAGGCTTTTTTGGAGGCCTAGGCTTTTGCA |
| | AAAAGCTCCCGGGAGCTTGTATATCCATTTTCGGATCTGATCAAGAGACAGGATG |
| | AGGATCGTTTCGCATGATTGAACAAGATGGATTGCACGCAGGTTCTCCGGCCGCT |
| | TGGGTGGAGAGGCTATTCGGCTATGACTGGGCACAACAGACAATCGGCTGCTCTG |
| | ATGCCGCCGTGTTCCGGCTGTCAGCGCAGGGGCGCCCGGTTCTTTTTGTCAAGAC |
| | CGACCTGTCCGGTGCCCTGAATGAACTGCAGGACGAGGCAGCGCGGCTATCGTGG |
| | CTGGCCACGACGGGCGTTCCTTGCGCAGCTGTGCTCGACGTTGTCACTGAAGCGG |
| | GAAGGGACTGGCTGCTATTGGGCGAAGTGCCGGGGCAGGATCTCCTGTCATCTCA |
| | CCTTGCTCCTGCCGAGAAAGTATCCATCATGGCTGATGCAATGCGGCGGCTGCAT |
| | ACGCTTGATCCGGCTACCTGCCCATTCGACCACCAAGCGAAACATCGCATCGAGC |
| | GAGCACGTACTCGGATGGAAGCCGGTCTTGTCGATCAGGATGATCTGGACGAAGA |
| | GCATCAGGGGCTCGCGCCAGCCGAACTGTTCGCCAGGCTCAAGGCGCGCATGCCC |
| | GACGGCGAGGATCTCGTCGTGACCCATGGCGATGCCTGCTTGCCGAATATCATGG |
| | TGGAAAATGGCCGCTTTTCTGGATTCATCGACTGTGGCCGGCTGGGTGTGGCGGA |
| | CCGCTATCAGGACATAGCGTTGGCTACCCGTGATATTGCTGAAGAGCTTGGCGGC |
| | GAATGGGCTGACCGCTTCCTCGTGCTTTACGGTATCGCCGCTCCCGATTCGCAGC |
| | GCATCGCCTTCTATCGCCTTCTTGACGAGTTCTTCTGAGCGGGACTCTGGGGTTC |
| | GCGAAATGAGCGAGCAAGCGAGGCCCAACCTGCCATCACGAGATTTCGATTCGAG |
| | CGCCGCCTTCTATGAAAGGTTGGGCTTCGGAATCGTTTTCCGGGACGCCGGCTGG |
| | ATGATCCTCCAGCGCGGGGATCTCATGCTGGAGTTCTTCGCCCACCCCAACTTGT |
| | TTATTGCAGCTTATAATGGTTACAAATAAAGCAATAGCATCACAAATTTCACAAA |
| | TAAAGCATTTTTTTCACTGCATTCTAGTTGTGGTTTGTCCAAACTCATCAATGTA |
| | TCTTATCATGTCTGTATACCGTCGACCTCTAGCTAGAGCTTGGCGTAATCATGGT |
| | CATAGCTGTTTCCTGTGTGAAATTGTTATCCGCTCACAATTCCACACAACATACG |
| | AGCCGGAAGCATAAAGTGTAAAGCCTGGGGTGCCTAATGAGTGAGCTAACTCACA |
| | TTAATTGCGTTGCGCTCACTGCCCGCTTTCCAGTCGGGAAACCTGTCGTGCCAGC |
| | TGCATTAATGAATCGGCCAACGCGCGGGGAGAGGCGGTTTGCGTATTGGGCGCTC |
| | TTCCGCTTCCTCGCTCACTGACTCGCTGCGCTCGGTCGTTCGGCTGCGGCGAGCG |
| | GTATCAGCTCACTCAAAGGCGGTAATACGGTTATCCACAGAATCAGGGGATAACG |
| | CAGGAAAGAACATGTGAGCAAAAGGCCAGCAAAAGGCCAGGAACCGTAAAAAGGC |
| | CGCGTTGCTGGCGTTTTTCCATAGGCTCCGCCCCCCTGACGAGCATCACAAAAAT |
| | CGACGCTCAAGTCAGAGGTGGCGAAACCCGACAGGACTATAAAGATACCAGGCGT |
| | TTCCCCCTGGAAGCTCCCTCGTGCGCTCTCCTGTTCCGACCCTGCCGCTTACCGG |
| | ATACCTGTCCGCCTTTCTCCCTTCGGGAAGCGTGGCGCTTTCTCATAGCTCACGC |
| | TGTAGGTATCTCAGTTCGGTGTAGGTCGTTCGCTCCAAGCTGGGCTGTGTGCACG |
| | AACCCCCCGTTCAGCCCGACCGCTGCGCCTTATCCGGTAACTATCGTCTTGAGTC |
| | CAACCCGGTAAGACACGACTTATCGCCACTGGCAGCAGCCACTGGTAACAGGATT |
| | AGCAGAGCGAGGTATGTAGGCGGTGCTACAGAGTTCTTGAAGTGGTGGCCTAACT |
| | ACGGCTACACTAGAAGAACAGTATTTGGTATCTGCGCTCTGCTGAAGCCAGTTAC |
| | CTTCGGAAAAAGAGTTGGTAGCTCTTGATCCGGCAAACAAACCACCGCTGGTAGC |
| | GGTGGTTTTTTTGTTTGCAAGCAGCAGATTACGCGCAGAAAAAAAGGATCTCAAG |
| | AAGATCCTTTGATCTTTTCTACGGGGTCTGACGCTCAGTGGAACGAAAACTCACG |
| | TTAAGGGATTTTGGTCATGAGATTATCAAAAAGGATCTTCACCTAGATCCTTTTA |
| | AATTAAAAATGAAGTTTTAAATCAATCTAAAGTATATATGAGTAAACTTGGTCTG |
| | ACAGTTACCAATGCTTAATCAGTGAGGCACCTATCTCAGCGATCTGTCTATTTCG |
| | TTCATCCATAGTTGCCTGACTCCCCGTCGTGTAGATAACTACGATACGGGAGGGC |
| | TTACCATCTGGCCCCAGTGCTGCAATGATACCGCGAGACCCACGCTCACCGGCTC |
| | CAGATTTATCAGCAATAAACCAGCCAGCCGGAAGGGCCGAGCGCAGAAGTGGTCC |
| | TGCAACTTTATCCGCCTCCATCCAGTCTATTAATTGTTGCCGGGAAGCTAGAGTA |
| | AGTAGTTCGCCAGTTAATAGTTTGCGCAACGTTGTTGCCATTGCTACAGGCATCG |
| | TGGTGTCACGCTCGTCGTTTGGTATGGCTTCATTCAGCTCCGGTTCCCAACGATC |
| | AAGGCGAGTTACATGATCCCCCATGTTGTGCAAAAAAGCGGTTAGCTCCTTCGGT |
| | CCTCCGATCGTTGTCAGAAGTAAGTTGGCCGCAGTGTTATCACTCATGGTTATGG |
| | CAGCACTGCATAATTCTCTTACTGTCATGCCATCCGTAAGATGCTTTTCTGTGAC |
| | TGGTGAGTACTCAACCAAGTCATTCTGAGAATAGTGTATGCGGCGACCGAGTTGC |
| | TCTTGCCCGGCGTCAATACGGGATAATACCGCGCCACATAGCAGAACTTTAAAAG |
| | TGCTCATCATTGGAAAACGTTCTTCGGGGCGAAAACTCTCAAGGATCTTACCGCT |
| | GTTGAGATCCAGTTCGATGTAACCCACTCGTGCACCCAACTGATCTTCAGCATCT |
| | TTTACTTTCACCAGCGTTTCTGGGTGAGCAAAAACAGGAAGGCAAAATGCCGCAA |
| | AAAAGGGAATAAGGGCGACACGGAAATGTTGAATACTCATACTCTTCCTTTTTCA |
| | ATATTATTGAAGCATTTATCAGGGTTATTGTCTCATGAGCGGATACATATTTGAA |
| | TGTATTTAGAAAAATAAACAAATAGGGGTTCCGCGCACATTTCCCCGAAAAGTGC |
| | CACCTGACGTCGACGGATCGGGAGATCTCCCGATCCCCTATGGTCGACTCTCAGT |
| | ACAATCTGCTCTGATGCCGCATAGTTAAGCCAGTATCTGCTCCCTGCTTGTGTGT |
| | TGGAGGTCGCTGAGTAGTGCGCGAGCAAAATTTAAGCTACAACAAGGCAAGGCTT |
| | GACCGACAATTGCATGAAGAATCTGCTTAGG (SEQ ID NO: 284) |
| AB1783 | GAGGTGCAGCTGGTTGAATCTGGCGGCGGACTTGTGAAGCCTGGCGGATCTCTGA |
| A49MI- | GACTGTCTTGTGCCGCCTCCGGCTTCACCTTCTCCAGCTACTCTATGAACTGGGT |
| VH-CH1- | CCGACAGGCCCCTGGCAAAGGACTGGAATGGGTGTCCTCTATCTCCTCCAGCTCC |
| Fc | TCCTACATCTACTACGCCGACTCCGTGAAGGGCAGATTCACCATCAGCCGGGACA |
| | ACGCCAAGAACTCCCTGTACCTGCAGATGAACAGCCTGAGAGCCGAGGACACCGC |
| | CGTGTACTATTGTGCTAGAGGCGCTCCTATCGGCGCTGCCGCTGGATGGTTCGAT |
| | CCTTGGGGACAGGGAACCCTGGTCACCGTGTCCTCTGCTTCTACCAAGGGACCCA |
| | GCGTGTTCCCTCTGGCTCCTTCCAGCAAGTCTACCTCTGGCGGAACAGCTGCTCT |
| | GGGCTGCCTGGTCAAGGACTACTTTCCTGAGCCTGTGACCGTGTCTTGGAACTCT |
| | GGCGCTCTGACATCCGGCGTGCACACATTTCCAGCTGTGCTGCAGTCCTCCGGCC |
| | TGTACTCTCTGTCCTCTGTCGTGACCGTGCCTTCCAGCTCTCTGGGAACCCAGAC |
| | CTACATCTGCAATGTGAACCACAAGCCTTCCAACACCAAGGTGGACAAGAAGGTG |
| | GAACCCAAGTCCTGCGACAAGACCCACACCTGTCCTCCATGTCCTGCTCCAGAAC |
| | TGCTCGGCGGACCTTCCGTGTTCCTGTTTCCTCCAAAGCCTAAGGACACCCTGAT |
| | GATCTCTCGGACCCCTGAAGTGACCTGCGTGGTGGTGGATGTGTCTCACGAGGAT |
| | CCCGAAGTGAAGTTCAATTGGTACGTGGACGGCGTGGAAGTGCACAATGCCAAGA |
| | CCAAGCCTAGAGAGGAACAGTACAACTCCACCTACAGAGTGGTGTCCGTGCTGAC |
| | CGTGCTGCACCAGGATTGGCTGAACGGCAAAGAGTACAAGTGCAAGGTGTCCAAC |
| | AAGGCCCTGCCTGCTCCAATCGAAAAGACCATCTCCAAGGCCAAGGGCCAGCCTC |
| | GGGAACCTCAAGTCTGTACCTTGCCTCCTAGCCGGGATGAGCTGACCGAGAATCA |
| | GGTGTCCCTGACCTGCCTCGTGAAGGGATTCTACCCCTCCGATATCGCCGTGGAA |
| | TGGGAGTCTAATGGCCAGCCAGAGAACAACTACAAGACAACCCCTCCTGTGCTGG |
| | ACTCCGACGGCTCCTTCTTTCTGTACTCCTGGCTGACAGTGGACAAGTCCAGATG |
| | GCAGCAGGGCAACGTGTTCTCCTGCTCCGTGATGCACGAGGCCCTGCACAATCAC |
| | TACACCCAGAAGTCCCTGTCTCTGTCCCCTGGC (SEQ ID NO: 549) |
| AB1783 | GTTAGGCGTTTTGCGCTGCTTCGCGATGTACGGGCCAGATATACGCGTTGACATT |
| A49MI- | GATTATTGAGTAGTTATTAATAGTAATCAATTACGGGGTCATTAGTTCATAGCCC |
| VL-CL | ATATATGGAGTTCCGCGTTACATAACTTACGGTAAATGGCCCGCCTGGCTGACCG |
| | CCCAACGACCCCCGCCCATTGACGTCAATAATGACGTATGTTCCCATAGTAACGC |
| | CAATAGGGACTTTCCATTGACGTCAATGGGTGGAGTATTTACGGTAAACTGCCCA |
| | CTTGGGAGTAGATCAAGTGTATCATATGCCAAGTACGCCCCCTATTGAGGTCAAT |
| | GACGGTAAATGGCCCGCCTGGCATTATGCCCAGTACATGACCTTATGGGACTTTC |
| | CTACTTGGCAGTACATCTACGTATTAGTCATCGCTATTACCATGGTGATGCGGTT |
| | TTGGCAGTACATCAATGGGCGTGGATAGCGGTTTGACTCACGGGGATTTCCAAGT |
| | CTCCACCCCATTGACGTCAATGGGAGTTTGTTTTGGCACCAAAATCAACGGGACT |
| | TTCCAAAATGTCGTAACAACTCCGCCCCATTGACGCAAATGGGCGGTAGGCGTGT |
| | ACGGTGGGAGGTCTATATAAGCAGAGCTCGTTTAGTGAACCGTCAGATCGCCTGG |
| | AGACGCCATCCACGCTGTTTTGACCTCCATAGAAGACACCGGGACCGATCCAGCC |
| | TCCGGACTCTAGAGGATCGAACCCTTAAGCTTACCGCCACCATGGACATGAGAGT |
| | GCCCGCTCAGCTGCTGGGACTGCTGTTGTTGTGGTTGCCTGGCGCTAGATGCGAC |
| | ATCCAGATGACCCAGTCTCCATCCTCCGTGTCTGCCTCTGTGGGCGACAGAGTGA |
| | CCATCACCTGTAGAGCCAGCCAGGGCATCTCTTCTTGGCTGGCCTGGTATCAGCA |
| | GAAGCCTGGCAAGGCTCCCAAGCTGCTGATCTACGCTGCTTCCAGTCTGCAGTCT |
| | GGCGTGCCCTCCAGATTTTCCGGCTCTGGCTCTGGCACCGACTTTACCCTGACAA |
| | TCTCCAGCCTGCAGCCTGAGGACTTCGCCACCTACTATTGTCAGCAGGGCGTGTC |
| | CTTTCCTCGGACCTTTGGCGGAGGCACCAAGGTGGAAATCAAGCGGACAGTGGCC |
| | GCTCCTTCCGTGTTCATCTTCCCACCTTCCGACGAGCAGCTGAAGTCCGGCACAG |
| | CTTCTGTCGTGTGCCTGCTGAACAACTTCTACCCTCGGGAAGCCAAGGTGCAGTG |
| | GAAGGTGGACAATGCTCTGCAGTCCGGCAACTCCCAAGAGTCCGTGACCGAGCAG |
| | GACTCCAAGGACTCTACCTACAGCCTGTCCTCCACACTGACCCTGTCCAAGGCCG |
| | ACTACGAGAAGCACAAGGTGTACGCCTGCGAAGTGACCCATCAGGGCCTGTCTAG |
| | CCCTGTGACCAAGTCTTTCAACCGGGGCGAGTGCTGATGATCTAGAAAGGGTTCG |
| | ATCCCTACCGGTTAGTAATGAGTTTGATATCTCGACAATCAAGCTCTGGATTACA |
| | AAATTTGTGAAAGATTGACTGGTATTCTTAACTATGTTGCTCCTTTTACGCTATG |
| | TGGATACGCTGCTTTAATGCCTTTGTATCATGCTATTGCTTCCCGTATGGCTTTC |
| | ATTTTCTCCTCCTTGTATAAATCCTGGTTGCTGTCTCTTTATGAGGAGTTGTGGC |
| | CCGTTGTCAGGCAACGTGGCGTGGTGTGCACTGTGTTTGCTGACGCAACCCCCAC |
| | TGGTTGGGGCATTGCCACCACCTGTCAGCTCCTTTCCGGGACTTTCGCTTTCCCC |
| | CTCCCTATTGCCACGGCGGAACTCATCGCCGCCTGCCTTGCCCGCTGCTGGACAG |
| | GGGCTCGGCTGTTGGGCACTGACAATTCCGTGGTGTTGTCGGGGAAGCTGACGTC |
| | CTTTCCATGGCTGCTCGCCTGTGTTGCCACCTGGATTCTGCGCGGGACGTCCTTC |
| | TGCTACGTCCCTTCGGCCCTCAATCCAGCGGACCTTCCTTCCCGCGGCCTGCTGC |
| | CGGCTCTGCGGCCTCTTCCGCGTCTTCGCCTTCGCCCTCAGACGAGTCGGATCTC |
| | CCTTTGGGCCGCCTCCCCGCCTGGAACGGGGGAGGCTAACTGAAACACGGAAGGA |
| | GACAATACCGGAAGGAACCCGCGCTATGACGGCAATAAAAAGACAGAATAAAACG |
| | CACGGGTGTTGGGTCGTTTGTTCATAAACGCGGGGTTCGGTCCCAGGGCTGGCAC |
| | TCTGTCGATACCCCACCGAGACCCCATTGGGGCCAATACGCCCGCGTTTCTTCCT |
| | TTTCCCCACCCCACCCCCCAAGTTCGGGTGAAGGCCCAGGGCTCGCAGCCAACGT |
| | CGGGGCGGCAGGCCCTGCCATAGCAGATCTGCGCAGCTGGGGCTCTAGGGGGTAT |
| | CCCCACGCGCCCTGTAGCGGCGCATTAAGCGCGGCGGGTGTGGTGGTTACGCGCA |
| | GCGTGACCGCTACACTTGCCAGCGCCCTAGCGCCCGCTCCTTTCGCTTTCTTCCC |
| | TTCCTTTCTCGCCACGTTCGCCGGCTTTCCCCGTCAAGCTCTAAATCGGGGGCTC |
| | CCTTTAGGGTTCCGATTTAGTGCTTTACGGCACCTCGACCCCAAAAAACTTGATT |
| | AGGGTGATGGTTCACGTAGTGGGCCATCGCCCTGATAGACGGTTTTTCGCCCTTT |
| | GACGTTGGAGTCCACGTTCTTTAATAGTGGACTCTTGTTCCAAACTGGAACAACA |
| | CTCAACCCTATCTCGGTCTATTCTTTTGATTTATAAGGGATTTTGCCGATTTCGG |
| | CCTATTGGTTAAAAAATGAGCTGATTTAACAAAAATTTAACGCGAATTAATTCTG |
| | TGGAATGTGTGTCAGTTAGGGTGTGGAAAGTCCCCAGGCTCCCCAGCAGGCAGAA |
| | GTATGCAAAGCATGCATCTCAATTAGTCAGCAACCAGGTGTGGAAAGTCCCCAGG |
| | CTCCCCAGCAGGCAGAAGTATGCAAAGCATGCATCTCAATTAGTCAGCAACCATA |
| | GTCCCGCCCCTAACTCCGCCCATCCCGCCCCTAACTCCGCCCAGTTCCGCCCATT |
| | CTCCGCCCCATGGCTGACTAATTTTTTTTATTTATGCAGAGGCCGAGGCCGCCTC |
| | TGCCTCTGAGCTATTCCAGAAGTAGTGAGGAGGCTTTTTTGGAGGCCTAGGCTTT |
| | TGCAAAAAGCTCCCGGGAGCTTGTATATCCATTTTCGGATCTGATCAAGAGACAG |
| | GATGAGGATCGTTTCGCATGATTGAACAAGATGGATTGCACGCAGGTTCTCCGGC |
| | CGCTTGGGTGGAGAGGCTATTCGGCTATGACTGGGCACAACAGACAATCGGCTGC |
| | TCTGATGCCGCCGTGTTCCGGCTGTCAGCGCAGGGGCGCCCGGTTCTTTTTGTCA |
| | AGACCGACCTGTCCGGTGCCCTGAATGAACTGCAGGACGAGGCAGCGCGGCTATC |
| | GTGGCTGGCCACGACGGGCGTTCCTTGCGCAGCTGTGCTCGACGTTGTCACTGAA |
| | GCGGGAAGGGACTGGCTGCTATTGGGCGAAGTGCCGGGGCAGGATCTCCTGTCAT |
| | CTCACCTTGCTCCTGCCGAGAAAGTATCCATCATGGCTGATGCAATGCGGCGGCT |
| | GCATACGCTTGATCCGGCTACCTGCCCATTCGACCACCAAGCGAAACATCGCATC |
| | GAGCGAGCACGTACTCGGATGGAAGCCGGTCTTGTCGATCAGGATGATCTGGACG |
| | AAGAGCATCAGGGGCTCGCGCCAGCCGAACTGTTCGCCAGGCTCAAGGCGCGCAT |
| | GCCCGACGGCGAGGATCTCGTCGTGACCCATGGCGATGCCTGCTTGCCGAATATC |
| | ATGGTGGAAAATGGCCGCTTTTCTGGATTCATCGACTGTGGCCGGCTGGGTGTGG |
| | CGGACCGCTATCAGGACATAGCGTTGGCTACCCGTGATATTGCTGAAGAGCTTGG |
| | CGGCGAATGGGCTGACCGCTTCCTCGTGCTTTACGGTATCGCCGCTCCCGATTCG |
| | CAGCGCATCGCCTTCTATCGCCTTCTTGACGAGTTCTTCTGAGCGGGACTCTGGG |
| | GTTCGCGAAATGACCGACCAAGCGACGCCCAACCTGCCATCACGAGATTTCGATT |
| | CCACCGCCGCCTTCTATGAAAGGTTGGGCTTCGGAATCGTTTTCCGGGACGCCGG |
| | CTGGATGATCCTCCAGCGCGGGGATCTCATGCTGGAGTTCTTCGCCCACCCCAAC |
| | TTGTTTATTGCAGCTTATAATGGTTACAAATAAAGCAATAGCATCACAAATTTGA |
| | CAAATAAAGCATTTTTTTCACTGCATTCTAGTTGTGGTTTGTCCAAACTCATCAA |
| | TGTATCTTATCATGTCTGTATACCGTCGACCTCTAGCTAGAGCTTGGCGTAATCA |
| | TGGTCATAGCTGTTTCCTGTGTGAAATTGTTATCCGCTCACAATTCCACACAACA |
| | TACGAGCCGGAAGCATAAAGTGTAAAGCCTGGGGTGCCTAATGAGTGAGCTAACT |
| | CACATTAATTGCGTTGCGCTCACTGCCCGCTTTCCAGTCGGGAAACCTGTCGTGC |
| | CAGCTGCATTAATGAATCGGCCAACGCGCGGGGAGAGGCGGTTTGCGTATTGGGC |
| | GCTCTTCCGCTTCCTCGCTCACTGACTCGCTGCGCTCGGTCGTTCGGCTGCGGCG |
| | AGCGGTATCAGCTCACTCAAAGGCGGTAATACGGTTATCCACAGAATCAGGGGAT |
| | AACGCAGGAAAGAACATGTGAGCAAAAGGCCAGCAAAAGGCCAGGAACCGTAAAA |
| | AGGCCGCGTTGCTGGCGTTTTTCCATAGGCTCCGCCCCCCTGACGAGCATCACAA |
| | AAATCGACGCTCAAGTCAGAGGTGGCGAAACCCGACAGGACTATAAAGATACCAG |
| | GCGTTTCCCCCTGGAAGCTCCCTCGTGCGCTCTCCTGTTCCGACCCTGCCGCTTA |
| | CCGGATACCTGTCCGCCTTTCTCCCTTCGGGAAGCGTGGCGCTTTCTCATAGCTC |
| | ACGCTGTAGGTATCTCAGTTCGGTGTAGGTCGTTCGCTCCAAGCTGGGCTGTGTG |
| | CACGAACCCCCCGTTCAGCCCGACCGCTGCGCCTTATCCGGTAACTATCGTCTTG |
| | AGTCCAACCCGGTAAGACACGACTTATCGCCACTGGCAGCAGCCACTGGTAACAG |
| | GATTAGCAGAGCGAGGTATGTAGGCGGTGCTACAGAGTTCTTGAAGTGGTGGCCT |
| | AACTACGGCTACACTAGAAGAACAGTATTTGGTATCTGCGCTCTGCTGAAGCCAG |
| | TTACCTTCGGAAAAAGAGTTGGTAGCTCTTGATCCGGCAAACAAACCACCGCTGG |
| | TAGCGGTGGTTTTTTTGTTTGCAAGCAGCAGATTACGCGCAGAAAAAAAGGATCT |
| | CAAGAAGATCCTTTGATCTTTTCTACGGGGTCTGACGCTCAGTGGAACGAAAACT |
| | GAGGTTAAGGGATTTTGGTCATGAGATTATCAAAAAGGATCTTCACCTAGATGCT |
| | TTTAAATTAAAAATGAAGTTTTAAATCAATCTAAAGTATATATGAGTAAACTTGG |
| | TCTGACAGTTACCAATGCTTAATCAGTGAGGCACCTATCTCAGCGATCTGTCTAT |
| | TTCGTTCATCCATAGTTGCCTGACTCCCCGTCGTGTAGATAACTACGATACGGGA |
| | GGGCTTACCATCTGGCCCCAGTGCTGCAATGATACCGCGAGACCCACGCTCACCG |
| | GCTCCAGATTTATCAGCAATAAACCAGCCAGCCGGAAGGGCCGAGCGCAGAAGTG |
| | GTCCTGCAACTTTATCCGCCTCCATCCAGTCTATTAATTGTTGCCGGGAAGCTAG |
| | AGTAAGTAGTTCGCCAGTTAATAGTTTGCGCAACGTTGTTGCCATTGCTACAGGC |
| | ATCGTGGTGTCACGCTCGTCGTTTGGTATGGCTTCATTCAGCTCCGGTTCCCAAC |
| | GATCAAGGCGAGTTACATGATCCCCCATGTTGTGCAAAAAAGCGGTTAGCTCCTT |
| | CGGTCCTCCGATCGTTGTCAGAAGTAAGTTGGCCGCAGTGTTATCACTCATGGTT |
| | ATGGCAGCACTGCATAATTCTCTTACTGTCATGCCATCCGTAAGATGCTTTTCTG |
| | TGACTGGTGAGTACTCAACCAAGTCATTCTGAGAATAGTGTATGCGGCGACCGAG |
| | TTGCTCTTGCCCGGCGTCAATACGGGATAATACCGCGCCACATAGCAGAACTTTA |
| | AAAGTGCTCATCATTGGAAAACGTTCTTCGGGGCGAAAACTCTCAAGGATCTTAC |
| | CGCTGTTGAGATCCAGTTCGATGTAACCCACTCGTGCACCCAACTGATCTTCAGC |
| | ATCTTTTACTTTCACCAGCGTTTCTGGGTGAGCAAAAACAGGAAGGCAAAATGCC |
| | GCAAAAAAGGGAATAAGGGCGACACGGAAATGTTGAATACTCATACTCTTCCTTT |
| | TTCAATATTATTGAAGCATTTATCAGGGTTATTGTCTCATGAGCGGATACATATT |
| | TGAATGTATTTAGAAAAATAAACAAATAGGGGTTCCGCGCACATTTCCCCGAAAA |
| | GTGCCACCTGACGTCGACGGATCGGGAGATCTCCCGATCCCCTATGGTCGACTCT |
| | CAGTACAATCTGCTCTGATGCCGCATAGTTAAGCCAGTATCTGCTCCCTGCTTGT |
| | GTGTTGGAGGTCGCTGAGTAGTGCGCGAGCAAAATTTAAGCTACAACAAGGCAAG |
| | GCTTGACCGACAATTGCATGAAGAATCTGCTTAGG (SEQ ID NO: 285) |
| AB1783 | GACATCCAGATGACCCAGTCTCCATCCTCCGTGTCTGCCTCTGTGGGCGACAGAG |
| A49MI- | TGACCATCACCTGTAGAGCCAGCCAGGGCATCTCTTCTTGGCTGGCCTGGTATCA |
| VL-CL | GCAGAAGCCTGGCAAGGCTCCCAAGCTGCTGATCTACGCTGCTTCCAGTCTGCAG |
| | TCTGGCGTGCCCTCCAGATTTTCCGGCTCTGGCTCTGGCACCGACTTTACCCTGA |
| | CAATCTCCAGCCTGCAGCCTGAGGACTTCGCCACCTACTATTGTCAGCAGGGCGT |
| | GTCCTTTCCTCGGACCTTTGGCGGAGGCACCAAGGTGGAAATCAAGCGGACAGTG |
| | GCCGCTCCTTCCGTGTTCATCTTCCCACCTTCCGACGAGCAGCTGAAGTCCGGCA |
| | CAGCTTCTGTCGTGTGCCTGCTGAACAACTTCTACCCTCGGGAAGCCAAGGTGCA |
| | GTGGAAGGTGGACAATGCTCTGCAGTCCGGCAACTCCCAAGAGTCCGTGACCGAG |
| | CAGGACTCCAAGGACTCTACCTACAGCCTGTCCTCCACACTGACCCTGTCCAAGG |
| | CCGACTACGAGAAGCACAAGGTGTACGCCTGCGAAGTGACCCATCAGGGCCTGTC |
| | TAGCCCTGTGACCAAGTCTTTCAACCGGGGCGAGTGC (SEQ ID NO: 550) |

In certain embodiments, a nucleic acid molecule of the present disclosure comprises SEQ ID NO:280. In certain embodiments, a nucleic acid molecule of the present disclosure comprises SEQ ID NO:281. In certain embodiments, a nucleic acid molecule of the present disclosure comprises SEQ ID NO:282. In certain embodiments, a nucleic acid molecule of the present disclosure comprises SEQ ID NO:283. In certain embodiments, a nucleic acid molecule of the present disclosure comprises SEQ ID NO:284. In certain embodiments, a nucleic acid molecule of the present disclosure comprises SEQ ID NO:285. In certain embodiments, a nucleic acid molecule of the present disclosure comprises SEQ ID NO:545. In certain embodiments, a nucleic acid molecule of the present disclosure comprises SEQ ID NO:546. In certain embodiments, a nucleic acid molecule of the present disclosure comprises SEQ ID NO:547. In certain embodiments, a nucleic acid molecule of the present disclosure comprises SEQ ID NO:548. In certain embodiments, a nucleic acid molecule of the present disclosure comprises SEQ ID NO:549. In certain embodiments, a nucleic acid molecule of the present disclosure comprises SEQ ID NO:550.

### II. Characteristics of the Multispecific Binding Proteins

The multispecific binding proteins described herein include an NKG2D-binding site, a 5T4 binding site, and an antibody Fc domain or a portion thereof sufficient to bind CD16, or an antigen-binding site that binds CD16. In some embodiments, the multispecific binding proteins contains an additional antigen-binding site that binds 5T4, as exemplified in the F4-TriNKET® format (e.g., FIGS. 2C and 2D).

In some embodiments, the multispecific binding proteins described herein, which include a binding site for 5T4, binds to a cell expressing 5T4 (e.g., a tumor/cancer cell, a cancer-associated fibroblast, or a cancer stem cell) with an EC₅₀ value of about 3 nM to about 52 nM (e.g., 3-52 nM, 3-51 nM, 3-50 nM, 3-49 nM, 3-48 nM, 3-47 nM, 3-46 nM, 3-45 nM, 3-44 nM, 3-43 nM, 3-42 nM, 3-41 nM, 3-40 nM, 3-39 nM, 3-38 nM, 3-37 nM, 3-36 nM, 3-35 nM, 3-34 nM, 3-33 nM, 3-32 nM, 3-31 nM, 3-30 nM, 3-29 nM, 3-28 nM, 3-27 nM, 3-26 nM, 3-25 nM, 3-24 nM, 3-23 nM, 3-22 nM, 3-21 nM, 3-20 nM, 3-19 nM, 3-18 nM, 3-17 nM, 3-16 nM, 3-15 nM, 3-14 nM, 3-13 nM, 3-12 nM, 3-11 nM, 3-10 nM, 3-9 nM, 3-8 nM, 3-7 nM, 3-6 nM, 3-5 nM, 3-4 nM, 4-52 nM, 5-52 nM, 6-52 nM, 7-52 nM, 8-52 nM, 9-52 nM, 10-52 nM, 11-52 nM, 12-52 nM, 13-52 nM, 14-52 nM, 15-52 nM, 16-52 nM, 17-52 nM, 18-52 nM, 19-52 nM, 20-52 nM, 21-52 nM, 22-52 nM, 23-52 nM, 24-52 nM, 25-52 nM, 26-52 nM, 27-52 nM, 28-52 nM, 29-52 nM, 30-52 nM, 31-52 nM, 32-52 nM, 33-52 nM, 34-52 nM, 35-52 nM, 36-52 nM, 37-52 nM, 38-52 nM, 39-52 nM, 40-52 nM, 41-52 nM, 42-52 nM, 43-52 nM, 44-52 nM, 45-52 nM, 46-52 nM, 47-52 nM, 48-52 nM, 49-52 nM, 50-52 nM, or 51-52 nM). In some embodiments, the multispecific binding proteins described herein (e.g., a protein comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 138, 139, and 140, respectively, and the VL comprises CDR1, CDR2, and CDR3 sequences comprising the amino acid sequences of SEQ ID NOs: 141, 142, and 143, respectively) binds to a cell expressing 5T4 (e.g., a tumor/cancer cell, a cancer-associated fibroblast, or a cancer stem cell) with an EC₅₀ value of about 3 nM-6 nM (e.g., 3 nM-5.5 nM, 3 nM-5.4 nM, 3 nM-5.3 nM, 3 nM-5.2 nM, 3 nM-4 nM, 3.1 nM-6 nM, 3.2 nM-6 nM, 3.3 nM-6 nM, 3.4 nM-6 nM, 3.5 nM-6 nM, 3.6 nM-6 nM, 3.7 nM-6 nM, 3.8 nM-6 nM, 3.9 nM-6 nM, 4 nM-6 nM, 4.1 nM-6 nM, 4.2 nM-6 nM, 4.3 nM-6 nM, 4.4 nM-6 nM, 4.5 nM-6 nM, 4.6 nM-6 nM, 4.7 nM-6 nM, 4.8 nM-6 nM, 4.9 nM-6 nM, 5 nM-6 nM, 5.1 nM-6 nM, 5.2 nM-6 nM, 5.3 nM-6 nM, 5.4 nM-6 nM, 5.5 nM-6 nM, 5.6 nM-6 nM, 5.7 nM-6 nM, 5.8 nM-6 nM, or 5.9 nM-6 nM).

In some embodiments, the multispecific binding proteins display similar thermal stability to the corresponding monoclonal antibody, i.e., a monoclonal antibody containing the same 5T4 binding site as the one incorporated in the multispecific binding proteins.

In some embodiments, the multispecific binding proteins simultaneously bind to cells expressing NKG2D and/or CD16, such as NK cells, and cells expressing 5T4, such as certain tumor cells. Binding of the multispecific binding proteins to NK cells can enhance the activity of the NK cells toward destruction of the 5T4 expressing cells (e.g., 5T4 expressing tumor cells). It has been reported that NK cells exhibit more potent cytotoxicity against target cells that are stressed (see Chan et al., (2014) Cell Death Differ. 21(1):5-14). Without wishing to be bound by theory, it is hypothesized that when NK cells are engaged to a population of cells by a TriNKET®, the NK cells may selectively kill the target cells that are stressed (e.g., malignant cells and cells in a tumor microenvironment). This mechanism could contribute to increased specificity and reduced toxicity of TriNKETs®, making it possible to selectively clear the stressed cells even if expression of 5T4 is not limited to the desired target cells.

In some embodiments, the multispecific binding proteins bind to 5T4 with a similar affinity to the corresponding the anti-5T4 monoclonal antibody (i.e., a monoclonal antibody containing the same 5T4 binding site as the one incorporated in the multispecific binding proteins). In some embodiments, the multispecific binding proteins are more effective in killing the tumor cells expressing 5T4 than the corresponding monoclonal antibodies.

In certain embodiments, the multispecific binding proteins described herein, which include a binding site for 5T4, activate primary human NK cells when co-culturing with cells expressing 5T4. NK cell activation is marked by the increase in CD107a degranulation and IFN-γ cytokine production. Furthermore, compared to a corresponding anti-5T4 monoclonal antibody, the multispecific binding proteins can show superior activation of human NK cells in the presence of cells expressing 5T4.

In some embodiments, the multispecific binding proteins described herein, which include a binding site for 5T4, enhance the activity of rested and IL-2-activated human NK cells when co-culturing with cells expressing 5T4.

In some embodiments, compared to the corresponding monoclonal antibody that binds to 5T4, the multispecific binding proteins offer an advantage in targeting tumor cells that express medium and low levels of 5T4.

In some embodiments, the bivalent F4 format of the TriNKETs® (i.e., TriNKETs® include an additional antigen-binding site that binds to 5T4) improve the avidity with which the TriNKETs® bind to 5T4. In some embodiments, the F4-TriNKETs® mediate more potent killing of tumor cells than the corresponding F3-TriNKETs® or F3′-TriNKETs®.

### III. Therapeutic Applications

The present application also describes methods for treating cancer using a multispecific binding protein described herein and/or a pharmaceutical composition described herein. Such methods include administering to a subject in need thereof an effective amount of any one of the multispecific binding proteins described herein, including administering to a subject in need thereof the multispecific binding protein in the form of an effective amount of the protein, or a pharmaceutical composition, formulation, or dosage thereof described herein. The multispecific binding proteins can be administered to a subject using any route well known in the art for administration of an antibody or antibody fragment, including without limitation intravenous and subcutaneous administration. The methods of the present application can improve a variety of clinical endpoints. For example, in some embodiments, the method increases overall survival in the subject relative to individuals not receiving treatment. In some embodiments, the method increases progression free survival in the subject relative to individuals not receiving treatment. In some embodiments, the method increases overall survival and progression free survival in the subject relative to individuals not receiving treatment.

The methods of the present application may be used to treat a variety of cancers expressing 5T4. Accordingly, in some embodiments, the 5T4 is expressed by cancer cells. In some embodiments, the 5T4 is expressed by cancer-associated fibroblasts. In some embodiments, the 5T4 is expressed at high levels relative to normal cells. In some embodiments, the 5T4 is expressed at low levels relative to normal cells.

The therapeutic method can be characterized according to the cancer to be treated. The cancer to be treated can be characterized according to the presence of a particular antigen expressed on the surface of the cancer cell, e.g., 5T4.

Cancers characterized by the expression of 5T4, include, without limitation, colorectal cancer, ovarian cancer, cervical cancer, lung (e.g., non-small cell lung cancer), renal cancer, bladder cancer, prostate cancer, breast cancer (e.g., hormone receptor positive (HR+) breast cancer), uterine cancer, endometrial cancer, squamous cell carcinoma, head and neck squamous cell carcinoma, uterine cancer, pancreatic cancer, mesothelioma, esophageal cancer, and gastric cancer. See, e.g., Stern, et al., Cancer Immunol Immunother (2017) 66:415-426.

It is contemplated that the protein, conjugate, cells, and/or pharmaceutical compositions described in the present disclosure can be used to treat a variety of cancers, not limited to cancers in which the cancer cells or the cells in the cancer microenvironment express 5T4. It is also contemplated that the subject treated with the protein, conjugate, cells, and/or pharmaceutical compositions described in the present disclosure has previously received treatment, including chemotherapy for cancer. As such, in some embodiments, the subject treated by the protein, conjugate, cells, and/or pharmaceutical compositions described in the present disclosure is refractory to chemotherapy.

In certain embodiments, the cancer is a solid tumor. In certain embodiments, the cancer is a metastatic cancer. In certain other embodiments, the cancer is brain cancer, bladder cancer, breast cancer (e.g., hormone receptor positive (HR+) breast cancer), cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, Bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma, nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain embodiments, the cancer is selected from the group consisting of colorectal cancer, ovarian cancer, cervical cancer, lung (e.g., non-small cell lung cancer), renal cancer, bladder cancer, prostate cancer, breast cancer (e.g., hormone receptor positive (HR+) breast cancer), uterine cancer, endometrial cancer, squamous cell carcinoma, head and neck squamous cell carcinoma, uterine cancer, pancreatic cancer, mesothelioma, esophageal cancer, and gastric cancer. In certain embodiments, the cancer is selected from the group consisting of breast cancer, cervical cancer, lung (e.g., non-small cell lung cancer), renal cancer, bladder cancer, head and neck squamous cell carcinoma, pancreatic cancer and gastric cancer.

### IV. Combination Therapy

Another aspect of the present application provides for combination therapy. A multispecific binding protein described herein can be used in combination with additional therapeutic agents to treat cancer.

Exemplary therapeutic agents that may be used as part of a combination therapy in treating cancer include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma (IFN-7), colony stimulating factor-1, colony stimulating factor-2, denileukin diftitox, interleukin-2, luteinizing hormone releasing factor and variations of the aforementioned agents that may exhibit differential binding to its cognate receptor, or increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine, e.g., selected from IL-2, IL-12, IL-15, GM-CSF, G-CSF, and variants thereof.

In some embodiments, the 5T4-targeting TriNKET® (anti-5T4×NKG2D×CD16 multispecific molecule), as described herein, are co-administered with one or more therapeutic agents selected from a PI3K inhibitor, a FLT3R agonist, a PD-1 antagonist, a PD-Li antagonist, a CD47 inhibitor, a Trop-2 inhibitor, an MCL1 inhibitor, a CCR8 binding agent, an HPK1 antagonist, a DGKα inhibitor, a CISH inhibitor, a PARP-7 inhibitor, a Cbl-b inhibitor, a KRAS inhibitor (e.g., a KRAS G12C or G12D inhibitor), a KRAS degrader, a beta-catenin degrader, a helios degrader, a CD73 inhibitor, an adenosine receptor antagonist, a TIGIT antagonist, a TREM1 binding agent, a TREM2 binding agent, a CD137 agonist, a GITR binding agent, an OX40 binding agent, and a CAR-T cell therapy.

In some embodiments, the 5T4-targeting TriNKET® (anti-5T4×NKG2D×CD16 multispecific molecule), as described herein, are co-administered with one or more therapeutic agents selected from a PI3K6 inhibitor (e.g., idealisib), a FLT3L-Fc fusion protein (e.g., GS-3583), an anti-PD-1 antibody (pembrolizumab, nivolumab, zimberelimab), a small molecule PD-L1 inhibitor (e.g., GS-4224), an anti-PD-L1 antibody (e.g., atezolizumab, avelumab), a CD47 inhibitor (e.g., magrolimab), a Trop-2 inhibitor (e.g., sacituzumab govitecan (TRODELVY™)), a small molecule MCL1 inhibitor (e.g., GS-9716), a small molecule HPK1 inhibitor (e.g., GS-6451), a HPK1 degrader (PROTAC; e.g., ARV-766), a small molecule DGKα inhibitor, a small molecule CD73 inhibitor (e.g., quemliclustat (AB680)), an anti-CD73 antibody (e.g., oleclumab), a dual A2a/A2b adenosine receptor antagonist (e.g., etrumadenant (AB928)), an anti-TIGIT antibody (e.g., tiragolumab, vibostolimab, domvanalimab, AB308), an anti-TREM1 antibody (e.g., PY159), an anti-TREM2 antibody (e.g., PY314), a CD137 agonist (e.g., AGEN-2373), a GITR/OX40 binding agent (e.g., AGEN-1223), an IL-2 receptor agonist (e.g., GS-4528) and a CAR-T cell therapy (e.g., axicabtagene ciloleucel, brexucabtagene autoleucel, tisagenlecleucel).

In some embodiments, the 5T4-targeting TriNKET® (anti-5T4×NKG2D×CD16 multispecific molecule), as described herein, are co-administered with one or more therapeutic agents selected from magrolimab, sacituzumab govitecan (TRODELVY™), GS-4528, idealisib, GS-3583, zimberelimab, GS-4224, GS-9716, GS-6451, quemliclustat (AB680), etrumadenant (AB928), domvanalimab, AB308, PY159, PY314, AGEN-1223, AGEN-2373, axicabtagene ciloleucel and brexucabtagene autoleucel.

In various embodiments, the 5T4-targeting TriNKET® (anti-5T4×NKG2D×CD16 multispecific molecule), as described herein, is further combined with an agent that inhibits binding between CD47 and SIRPα. In various embodiments, the agent that inhibits binding between CD47 and SIRPPα is an antibody or antigen-binding fragment thereof that binds to CD47 (a.k.a., IAP, MER6, OA3; NCBI Gene ID: 961; UniProt Q08722). In various embodiments, an antibody that binds to CD47 has an Fc having effector function. In various embodiments, an antibody that binds to CD47 is an IgG4 or an IgG1. Examples of anti-CD47 antibodies of use include without limitation: magrolimab, lemzoparlimab, letaplimab, ligufalimab (AK117), AO-176, IBI-322, ZL-1201, IMC-002, SRF-231, CC-90002 (a.k.a., INBRX-103), NI-1701 (a.k.a., TG-1801), STI-6643 (Vx-1004), CNTO-7108, RCT-1938, RRx-001, DSP-107, VT-1021 and SGN-CD47M.

In various embodiments, the agent that inhibits binding between CD47 and SIRPα CD47 is an antibody or antigen-binding fragment thereof that binds to signal regulatory protein alpha (SIRPα) (NCBI Gene ID: 140885; UniProt P78324). Illustrative antibodies that bind to SIRPα include without limitation: anzurstobart (a.k.a., CC-95251), GS-0189 (FSI-189), ES-004, BI765063 and ADU1805.

In various embodiments, the agent that inhibits binding between CD47 and SIRPα CD47 is a SIRPα-Fc fusion protein or a “high affinity SIRPα reagent”, which includes SIRPα-derived polypeptides and analogs thereof. Illustrative SIRPα-Fc fusion proteins of use include ALX-148 (a.k.a., evorpacept, described in WO2013109752), TTI-621 or TTI-622 (described in WO2014094122), SIRPα-F8, JY002-M2G1(N297A), JMT601 (CPO107), SS002M91, SIRPalpha-lgG4-Fc-Fc, and hCD172a(SIRPα)-Fc-LIGHT.

In various embodiments, the 5T4-targeting TriNKET® (anti-5T4×NKG2D×CD16 multispecific molecule), as described herein, is further combined with an agonist of fms related receptor tyrosine kinase 3 (FLT3); FLK2; STK1; CD135; FLK-2; NCBI Gene ID: 2322). Examples of FLT3 agonists include, but are not limited to, CDX-301 and GS-3583. GS-3583 is described, e.g., in WO 2020/263830, hereby incorporated herein by reference in its entirety for all purposes.

In various embodiments, the 5T4-targeting TriNKET® (anti-5T4×NKG2D×CD16 multispecific molecule), as described herein, is further combined with an anti-CD19 agent or antibody. Examples of anti-CD19 agents or antibodies that can be co-administered include without limitation: blinatumomab, tafasitamab, XmAb5574 (Xencor), AFM-11, inebilizumab, loncastuximab, MEDI 551 (Cellective Therapeutics); and MDX-1342 (Medarex).

In various embodiments, the 5T4-targeting TriNKET® (anti-5T4×NKG2D×CD16 multispecific molecule), as described herein, is further combined with an anti-CD20 agent or antibody. Examples of anti-CD20 agents or antibodies that can be co-administered include without limitation: IGN-002, PF-05280586; Rituximab (Rituxan/Biogen Idec), Ofatumumab (Arzerra/Genmab), Obinutuzumab (Gazyva/Roche Glycart Biotech), Alemtuzumab, Veltuzumab, Veltuzumab, Ocrelizumab (Ocrevus/Biogen Idec; Genentech), Ocaratuzumab and Ublituximab, and LFB-R603 (LFB Biotech.; rEVO Biologics).

In various embodiments, the 5T4-targeting TriNKET® (anti-5T4×NKG2D×CD16 multispecific molecule), as described herein, is further combined with an anti-CD22 agent or antibody. Examples of anti-CD22 agents or antibodies that can be co-administered include without limitation: Epratuzumab, AMG-412, and IMMU-103 (Immunomedics).

In various embodiments, the 5T4-targeting TriNKET® (anti-5T4×NKG2D×CD16 multispecific molecule), as described herein, is further combined with an anti-CD30 agent or antibody. Examples of anti-CD30 agents or antibodies that can be co-administered include without limitation: Brentuximab vedotin (Seattle Genetics).

In various embodiments, the 5T4-targeting TriNKET® (anti-5T4×NKG2D×CD16 multispecific molecule), as described herein, is further combined with an anti-CD33 agent or antibody. Examples of anti-CD33 agents or antibodies that can be co-administered include without limitation: gemtuzumab, lintuzumab, vadastuximab, CIK-CAR.CD33; CD33CART, AMG-330 (CD33/CD3), AMG-673 (CD33/CD3), GEM-333 (CD3/CD33), and IMGN-779.

In various embodiments, the 5T4-targeting TriNKET® (anti-5T4×NKG2D×CD16 multispecific molecule), as described herein, is further combined with an anti-CD37 agent or antibody. Examples of anti-CD37 agents or antibodies that can be co-administered include without limitation: BI836826 (Boehringer Ingelheim), Otlertuzumab, and TRU-016 (Trubion Pharmaceuticals).

In various embodiments, the 5T4-targeting TriNKET® (anti-5T4×NKG2D×CD16 multispecific molecule), as described herein, is further combined with an anti-CD38 agent or antibody. Examples of anti-CD38 agents or antibodies that can be co-administered include without limitation: CD38, such as T-007, UCART-38; Darzalex (Genmab), Daratumumab, JNJ-54767414 (Darzalex/Genmab), Isatuximab, SAR650984 (ImmunoGen), MOR202, MOR03087 (MorphoSys), TAK-079; and anti-CD38-attenukine, such as TAK573.

In various embodiments, the 5T4-targeting TriNKET® (anti-5T4×NKG2D×CD16 multispecific molecule), as described herein, is further combined with an anti-CD52 agent or antibody. Examples of anti-CD52 agents or antibodies that can be co-administered include without limitation: anti-CD52 antibodies, such as Alemtuzumab (Campath/University of Cambridge).

In various embodiments, the 5T4-targeting TriNKET® (anti-5T4×NKG2D×CD16 multispecific molecule), as described herein, is further combined with an anti-CD98 (4F2, FRP-1) agent or antibody. Examples of anti-CD98 agents or antibodies that can be co-administered include without limitation: IGN523 (Igenica).

In various embodiments, the 5T4-targeting TriNKET® (anti-5T4×NKG2D×CD16 multispecific molecule), as described herein, is further combined with an anti-CD157 (BST-1) agent or antibody. Examples of anti-CD157 agents or antibodies that can be co-administered include without limitation: OBT357, and MEN1112 (Menarini; Oxford BioTherapeutics).

In various embodiments, the 5T4-targeting TriNKET® (anti-5T4×NKG2D×CD16 multispecific molecule), as described herein, is further combined with an anti-DKK-1 agent or antibody. Examples of anti-DKK-1 agents or antibodies that can be co-administered include without limitation: BHQ880 (MorphoSys; Novartis), and DKN-01, LY-2812176 (Eli Lilly).

In various embodiments, the 5T4-targeting TriNKET® (anti-5T4×NKG2D×CD16 multispecific molecule), as described herein, is further combined with an anti-GRP78 (BiP) agent or antibody. Examples of anti-GRP78 agents or antibodies that can be co-administered include without limitation: PAT-SM6 (OncoMab GmbH).

In various embodiments, the 5T4-targeting TriNKET® (anti-5T4×NKG2D×CD16 multispecific molecule), as described herein, is further combined with an anti-NOTCH1 agent or antibody. Examples of anti-NOTCH1 agents or antibodies that can be co-administered include without limitation: Brontictuzumab, and OMP-52M51 (OncoMed Pharmaceuticals).

In various embodiments, the 5T4-targeting TriNKET® (anti-5T4×NKG2D×CD16 multispecific molecule), as described herein, is further combined with an anti-ROR1 agent or antibody. Examples of anti-ROR1 agents or antibodies that can be co-administered include without limitation: Mapatumumab, TRM1, and HGS-1012 (Cambridge Antibody Technology).

In various embodiments, the 5T4-targeting TriNKET® (anti-5T4×NKG2D×CD16 multispecific molecule), as described herein, is further combined with an anti-SLAMF7 (CS1, CD319) agent or antibody. Examples of anti-SLAMF7 agents or antibodies that can be co-administered include without limitation: Elotuzumab, HuLuc63, BMS-901608 (Empliciti/PDL BioPharma), and Mogamulizumab (KW-0761).

In various embodiments, the 5T4-targeting TriNKET® (anti-5T4×NKG2D×CD16 multispecific molecule), as described herein, is further combined with an anti-TNFRSF10A (DR4; APO2; CD261; TRAILR1; TRAILR-1) agent or antibody. Examples of anti-TNFRSF10A agents or antibodies that can be co-administered include without limitation: Mapatumumab, TRM1, and HGS-1012 (Cambridge Antibody Technology).

In various embodiments, the 5T4-targeting TriNKET® (anti-5T4×NKG2D×CD16 multispecific molecule), as described herein, is further combined with an anti-Transferrin Receptor (TFRC; CD71) agent or antibody. Examples of anti-Transferrin Receptor agents or antibodies that can be co-administered include without limitation: E2.3/A27.15 (University of Arizona).

In various embodiments, the 5T4-targeting TriNKET® (anti-5T4×NKG2D×CD16 multispecific molecule), as described herein, is further combined with an anti-EPHA3 agent or antibody. Examples of anti-EPHA3 agents or antibodies that can be co-administered include without limitation: Ifabotuzumab, and KB004 (Ludwig Institute for Cancer Research).

In various embodiments, the 5T4-targeting TriNKET® (anti-5T4×NKG2D×CD16 multispecific molecule), as described herein, is further combined with an anti-CCR4 agent or antibody. Examples of anti-CCR4 agents or antibodies that can be co-administered include without limitation: Mogamulizumab, and KW-0761 (Poteligeo/Kyowa Hakko Kirin Co.).

In various embodiments, the 5T4-targeting TriNKET® (anti-5T4×NKG2D×CD16 multispecific molecule), as described herein, is further combined with an anti-CXCR4 agent or antibody. Examples of anti-CXCR4 agents or antibodies that can be co-administered include without limitation: Ulocuplumab, BMS-936564, MDX-1338 (Medarex), and PF-06747143 (Pfizer).

In various embodiments, the 5T4-targeting TriNKET® (anti-5T4×NKG2D×CD16 multispecific molecule), as described herein, is further combined with an anti-BAFF agent or antibody. Examples of anti-BAFF agents or antibodies that can be co-administered include without limitation: Tabalumab, and LY2127399 (Eli Lilly).

In various embodiments, the 5T4-targeting TriNKET® (anti-5T4×NKG2D×CD16 multispecific molecule), as described herein, is further combined with an anti-BAFF Receptor (BAFF-R) agent or antibody. Examples of anti-BAFF-R agents or antibodies that can be co-administered include without limitation: VAY736 (MorphoSys; Novartis).

In various embodiments, the 5T4-targeting TriNKET® (anti-5T4×NKG2D×CD16 multispecific molecule), as described herein, is further combined with an anti-RANKL agent or antibody. Examples of anti-RANKL agents or antibodies that can be co-administered include without limitation: Denosumab, and AMG-162 (Prolia; Ranmark; Xgeva/Amgen).

In various embodiments, the 5T4-targeting TriNKET® (anti-5T4×NKG2D×CD16 multispecific molecule), as described herein, is further combined with an anti-IL-6 agent or antibody. Examples of anti-IL-6 agents or antibodies that can be co-administered include without limitation: Siltuximab, and CNTO-328 (Sylvant/Centocor).

In various embodiments, the 5T4-targeting TriNKET® (anti-5T4×NKG2D×CD16 multispecific molecule), as described herein, is further combined with an anti-IL-6 Receptor (IL-6R) agent or antibody. Examples of anti-IL-6R agents or antibodies that can be co-administered include without limitation: Tocilizumab, R-1569 (Actemra/Chugai Pharmaceutical; Osaka University), and AS-101 (CB-06-02, IVX-Q-101).

In various embodiments, the 5T4-targeting TriNKET® (anti-5T4×NKG2D×CD16 multispecific molecule), as described herein, is further combined with an anti-IL3RA (CD123) agent or antibody. Examples of anti-IL3RA (CD123) agents or antibodies that can be co-administered include without limitation: tagraxofusp, talacotuzumab (JNJ-56022473; CSL362 (CSL)), pivekimab sunirine (IMGN632), MB-102 (Mustang Bio), CSL360 (CSL); vibecotamab (XmAb14045; Xencor); KHK2823 (Kyowa Hakko Kirin Co.); MGD-024 (CD123/CD3; Macrogenics), APVO436 (CD123/CD3); flotetuzumab (CD123/CD3); JNJ-63709178 (CD123/CD3); and XmAb-14045 (CD123/CD3) (Xencor).

In various embodiments, the 5T4-targeting TriNKET® (anti-5T4×NKG2D×CD16 multispecific molecule), as described herein, is further combined with an anti-IL2RA (CD25) agent or antibody. Examples of anti-IL2RA agents or antibodies that can be co-administered include without limitation: Basiliximab, SDZ-CH1-621 (Simulect/Novartis), and Daclizumab.

In various embodiments, the 5T4-targeting TriNKET® (anti-5T4×NKG2D×CD16 multispecific molecule), as described herein, is further combined with an anti-IGF-1R (CD221) agent or antibody. Examples of anti-IGF-1R agents or antibodies that can be co-administered include without limitation: Ganitumab, AMG-479 (Amgen); Ganitumab, AMG-479 (Amgen), Dalotuzumab, MK-0646 (Pierre Fabre), and AVE1642 (ImmunoGen).

In various embodiments, the 5T4-targeting TriNKET® (anti-5T4×NKG2D×CD16 multispecific molecule), as described herein, is further combined with an anti-GM-CSF (CSF2) agent or antibody. Examples of anti-GM-CSF agents or antibodies that can be co-administered include without limitation: Lenzilumab (a.k.a., KB003; KaloBios Pharmaceuticals).

In various embodiments, the 5T4-targeting TriNKET® (anti-5T4×NKG2D×CD16 multispecific molecule), as described herein, is further combined with an anti-HGF agent or antibody. Examples of anti-HGF agents or antibodies that can be co-administered include without limitation: Ficlatuzumab, AV-299 (AVEO Pharmaceuticals).

In various embodiments, the 5T4-targeting TriNKET® (anti-5T4×NKG2D×CD16 multispecific molecule), as described herein, is further combined with an anti-CD44 agent or antibody. Examples of anti-CD44 agents or antibodies that can be co-administered include without limitation: RG7356, R05429083 (Chugai Biopharmaceuticals; Roche).

In various embodiments, the 5T4-targeting TriNKET® (anti-5T4×NKG2D×CD16 multispecific molecule), as described herein, is further combined with an anti-VLA-4 (CD49d) agent or antibody. Examples of anti-VLA-4 agents or antibodies that can be co-administered include without limitation: Natalizumab, and BG-0002-E (Tysabri/Elan Corporation).

In various embodiments, the 5T4-targeting TriNKET® (anti-5T4×NKG2D×CD16 multispecific molecule), as described herein, is further combined with an anti-ICAM-1 (CD54) agent or antibody. Examples of anti-ICAM-1 agents or antibodies that can be co-administered include without limitation: BI-505 (BioInvent International).

In various embodiments, the 5T4-targeting TriNKET® (anti-5T4×NKG2D×CD16 multispecific molecule), as described herein, is further combined with an anti-VEGF-A agent or antibody. Examples of anti-VEGF-A agents or antibodies that can be co-administered include without limitation: Bevacizumab (Avastin/Genentech; Hackensack University Medical Center).

In various embodiments, the 5T4-targeting TriNKET® (anti-5T4×NKG2D×CD16 multispecific molecule), as described herein, is further combined with an anti-Endosialin (CD248, TEM1) agent or antibody. Examples of anti-Endosialin agents or antibodies that can be co-administered include without limitation: Ontecizumab, and MORAB-004 (Ludwig Institute for Cancer Research; Morphotek).

In various embodiments, the 5T4-targeting TriNKET® (anti-5T4×NKG2D×CD16 multispecific molecule), as described herein, is further combined with an anti-CD79 agent or antibody. Examples of anti-CD79 agents or antibodies that can be co-administered include without limitation: polatuzumab, DCDS4501A, and RG7596 (Genentech).

In various embodiments, the 5T4-targeting TriNKET® (anti-5T4×NKG2D×CD16 multispecific molecule), as described herein, is further combined with an anti-Isocitrate dehydrogenase (IDH) agent or antibody. Examples of anti-IDH agents or antibodies that can be co-administered include without limitation: IDH1 inhibitor ivosidenib (Tibsovo; Agios) and the IDH2 inhibitor enasidenib (Idhifa; Celgene/Agios).

In various embodiments, the 5T4-targeting TriNKET® (anti-5T4×NKG2D×CD16 multispecific molecule), as described herein, is further combined with an antibody that targets tumor associated calcium signal transducer 2 (TACSTD2) (NCBI Gene ID: 4070; EGP-1, EGP1, GA733-1, GA7331, GP50, M1S1, TROP2), such as sacituzumab, e.g., sacituzumab govitecan (TRODELVY™).

In various embodiments, the 5T4-targeting TriNKET® (anti-5T4×NKG2D×CD16 multispecific molecule), as described herein, is further combined with an anti-major histocompatibility complex, class I, G (HLA-G; NCBI Gene ID: 3135) antibody, such as TTX-080.

In various embodiments, the 5T4-targeting TriNKET® (anti-5T4×NKG2D×CD16 multispecific molecule), as described herein, is further combined with an anti-leukocyte immunoglobulin like receptor B2 (LILRB2, a.k.a., CD85D, ILT4; NCBI Gene ID: 10288) antibody, such as JTX-8064 or MK-4830.

### TNF Receptor Superfamily (TNFRSF) Member Agonists or Activators

In various embodiments, the 5T4-targeting TriNKET® (anti-5T4×NKG2D×CD16 multispecific molecule), as described herein, is further combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1B, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628, each of which is hereby incorporated by reference in its entirety.

Examples anti-TNF receptor superfamily member 10b (TNFRSF10B, DR5, TRAILR2) antibodies that can be co-administered include without limitation: DS-8273, CTB-006, INBRX-109, and GEN-1029.

Examples of anti-TNFRSF5 (CD40) antibodies that can be co-administered include without limitation: selicrelumab (R07009789), mitazalimab (a.k.a., vanalimab, ADC-1013, JNJ-64457107), RG7876, SEA-CD40, APX-005M and ABBV-428, ABBV-927, and JNJ-64457107.

Examples of anti-TNFRSF7 (CD27) that can be co-administered include without limitation: varlilumab (CDX-1127).

Examples of anti-TNFRSF9 (4-1BB, CD137) antibodies that can be co-administered include without limitation: urelumab, utomilumab (PF-05082566), AGEN2373, and ADG-106, BT-7480, and QL1806.

Examples of anti-TNFRSF17 (BCMA) that can be co-administered include without limitation: GSK-2857916.

Examples of anti-TNFRSF18 (GITR) antibodies that can be co-administered include without limitation: MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628, each of which is hereby incorporated by reference in its entirety.

Example anti-TRAILR1, anti-TRAILR2, anti-TRAILR3, anti-TRAILR4 antibodies that can be co-administered include without limitation: ABBV-621.

Examples of bi-specific antibodies targeting TNFRSF family members that can be co-administered include without limitation: PRS-343 (CD-137/HER2), AFM26 (BCMA/CD16A), AFM-13 (CD16/CD30), REGN-1979 (CD20/CD3), AMG-420 (BCMA/CD3), INHIBRX-105 (4-1BB/PDL1), FAP-4-IBBL (4-1BB/FAP), XmAb-13676 (CD3/CD20), RG-7828 (CD20/CD3), CC-93269 (CD3/BCMA), REGN-5458 (CD3/BCMA), and IMM-0306 (CD47/CD20), and AMG-424 (CD38.CD3).

Examples of inhibitors of PVR related immunoglobulin domain containing (PVRIG, CD112R) that can be co-administered include without limitation: COM-701.

Examples of inhibitors of T cell immunoreceptor with Ig and ITIM domains (TIGIT; NCBI Gene ID: 201633) that can be co-administered include without limitation: BMS-986207, RG-6058, AGEN-1307, and COM-902, etigilimab, tiragolumab (a.k.a., MTIG-7192A; RG-6058; RO 7092284), AGEN1777, IBI-939, AB154, MG1131, and EOS884448 (EOS-448).

Examples of inhibitors of hepatitis A virus cellular receptor 2 (HAVCR2, TIMD3, TIM-3) that can be co-administered include without limitation: cobolimab (TSR-022), LY-3321367, sabatolimab (MBG-453), INCAGN-2390, RO-7121661 (PD-1/TIM-3), LY-3415244 (TIM-3/PDL1), and RG7769 (PD-1/TIM-3).

Examples of inhibitors of lymphocyte activating 3 (LAG-3, CD223) that can be co-administered include without limitation: relatlimab (ONO-4482), LAG-525, MK-4280, REGN-3767, INCAGN2385, TSR-033, MGD-013 (PD-1/LAG-3), and FS-118 (LAG-3/PD-L1).

Examples of anti-V-set immunoregulatory receptor (VSIR, B7H5, VISTA) antibodies that can be co-administered include without limitation: HM11BD-002, and CA-170 (PD-L1/VISTA).

Examples of anti-CD70 antibodies that can be co-administered include without limitation: AMG-172.

Examples of anti-ICOS antibodies that can be co-administered include without limitation: JTX-2011, and GSK3359609.

Examples of ICOS agonists that can be co-administered include without limitation: ICOS-L.COMP (Gariepy, et al. 106th Annu Meet Am Assoc Immunologists (AAI) (May 9-13, San Diego) 2019, Abst 71.5).

### Immune Checkpoint Inhibitors

In various embodiments, the 5T4-targeting TriNKET® (anti-5T4×NKG2D×CD16 multispecific molecule), as described herein, is further combined with one or more immune checkpoint inhibitors. In some embodiments, the one or more immune checkpoint inhibitors is a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4.

Examples of inhibitors of CTLA4 that can be co-administered include without limitation: ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, HBM-4003, as well as multi-specific inhibitors FPT-155 (CTLA4/PD-L1/CD28), PF-06936308 (PD 1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors/antibodies of PD-L1 (CD274) or PD-1 (PDCD1) that can be co-administered include without limitation: zimberelimab, pembrolizumab (KEYTRUDA®, MK-3477), nivolumab (OPDIVO®, BMS-936558, MDX-1106), cemiplimab, pidilizumab, spartalizumab (PDR-001), atezolizumab (RG 7446; TECENTRIQ, MPDL3280A), durvalumab (MEDI-4736), avelumab (MSB0010718C), tislelizumab (BGB-A317), toripalimab (JS-001), genolimzumab (CBT-501), camrelizumab (SHR-1210), dostarlimab (TSR-042), sintilimab (IBI-308), tislelizumab (BGB-A317), cemiplimab (REGN-2810), lambrolizumab (CAS Reg. No. 1374853-91-4), AMG-404, AMP-224, MEDI0680 (AMP-514), BMS-936559, CK-301, PF-06801591, GEN-1046 (PD-L1/4-1BB), GLS-010 (WBP-3055), AK-103 (HX-008), AK-105, CS-1003, HLX-10, MGA-012, BI-754091, AGEN-2034, JNJ-63723283, LZM-009, BCD-100, LY-3300054, SHR-1201, Sym-021, ABBV-181, PD1-PIK, BAT-1306, CX-072, CBT-502, MSB-2311, JTX-4014, BGB-A333, SHR-1316, CS-1001 (WBP-3155, KN-035, HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), RO-7247669 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM-3/PDL1), RG7769 (PD-1/TIM-3) and INBRX-105 (4-1BB/PDL1), GNS-1480 (PD-L1/EGFR), SCH-900475, PF-06801591, AGEN-2034, AK-105, PD1-PIK, BAT-1306, BMS-936559, CK-301, MEDI-0680, PDR001+Tafinlar®+Mekinist®, and those described, e.g., in Intl. Patent Publ. Nos. WO2018195321, WO2020014643, WO2019160882, and WO2018195321.

In various embodiments, the 5T4-targeting TriNKET® (anti-5T4×NKG2D×CD16 multispecific molecule), as described herein, is combined with an inhibitor of MCL1 apoptosis regulator, BCL2 family member (MCL1, TM; EAT; MCL1L; MCL1S; Mcl-1; BCL2L3; MCL1-ES; bcl2-L-3; mcl1/EAT; NCBI Gene ID: 4170). Examples of MCL1 inhibitors include AMG-176, AMG-397, S-64315, and AZD-5991, 483-LM, A-1210477, UMI-77, JKY-5-037, and those described in WO2018183418, WO2016033486, and WO2017147410.

### Toll-Like Receptor (TLR) Agonists

In various embodiments, the 5T4-targeting TriNKET® (anti-5T4×NKG2D×CD16 multispecific molecule), as described herein, is combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation: DS-0509, GS-9620, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An TLR7/TLR8 agonist that can be co-administered is NKTR-262. Example TLR8 agonists that can be co-administered include without limitation: E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include without limitation: AST-008, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, leftolimod (MGN-1703), CYT-003, CYT-003-QbG10, and PUL-042. Examples of TLR3 agonist include rintatolimod, poly-ICLC, RIBOXXON®, Apoxxim, RIBOXXIM®, IPH-33, MCT-465, MCT-475, and ND-1.1.

Examples of TLR8 inhibitors include, but are not limited to, E-6887, IMO-8400, IMO-9200, and VTX-763.

Examples of TLR8 agonists include, but are not limited to, MCT-465, motolimod, GS-9688, and VTX-1463.

Examples of TLR9 agonists include but are not limited to, AST-008, IMO 2055, IMO-2125, lefitolimod, litenimod, MGN-1601, and PUL-042.

Examples of TLR7/TLR8 agonists include without limitation: NKTR-262, IMO-4200, MEDI-9197 (telratolimod), and resiquimod.

Examples of TLR agonists include without limitation: lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, and telratolimod.

In some embodiments, the therapeutic agent is a stimulator of interferon genes (STING) In some embodiments, the STING receptor agonist or activator is selected from ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP), and cyclic-di-AMP.

### Hematopoietic Progenitor Kinase 1 (HPK1) Inhibitors

In various embodiments, the 5T4-targeting TriNKET® (anti-5T4×NKG2D×CD16 multispecific molecule), as described herein, is further combined with an inhibitor of mitogen-activated protein kinase kinase kinase kinase 1 (MAP4K1, HPK1; NCBI Gene ID: 11184). Examples of Hematopoietic Progenitor Kinase 1 (HPK1) inhibitors include without limitation, those described in WO-2018183956, WO-2018183964, WO-2018167147, WO-2018183964, WO-2016205942, WO-2018049214, WO-2018049200, WO-2018049191, WO-2018102366, WO-2018049152, WO2020092528, WO2020092621, and WO-2016090300.

### Apoptosis Signal-Regulating Kinase (ASK) Inhibitors

In various embodiments, the 5T4-targeting TriNKET® (anti-5T4×NKG2D×CD16 multispecific molecule), as described herein, is further combined with an inhibitor of an ASK inhibitor, e.g., mitogen-activated protein kinase kinase kinase 5 (MAP3K5; ASK1, MAPKKK5, MEKK5; NCBI Gene ID: 4217). Examples of ASK1 inhibitors include without limitation, those described in WO 2011/008709 (Gilead Sciences) and WO 2013/112741 (Gilead Sciences).

### Bruton Tyrosine Kinase (BTK) Inhibitors

In various embodiments, the 5T4-targeting TriNKET® (anti-5T4×NKG2D×CD16 multispecific molecule), as described herein, is further combined with an inhibitor of Bruton tyrosine kinase (BTK, AGMX1, AT, ATK, BPK, IGHD3, IMD1, PSCTK1, XLA; NCBI Gene ID: 695). Examples of BTK inhibitors include without limitation, (S)-6-amino-9-(1-(but-2-ynoyl)pyrrolidin-3-yl)-7-(4-phenoxyphenyl)-7H-purin-8(9H)-one, acalabrutinib (ACP-196), BGB-3111, CB988, HM71224, ibrutinib (Imbruvica), M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, TAS-5315, Calquence+AZD6738, and Calquence+danvatirsen.

### Cyclin-Dependent Kinase (CDK) Inhibitors

In various embodiments, the 5T4-targeting TriNKET® (anti-5T4×NKG2D×CD16 multispecific molecule), as described herein, is further combined with an inhibitor of cyclin dependent kinase 1 (CDK1, CDC2; CDC28A; P34CDC2; NCBI Gene ID: 983); cyclin dependent kinase 2 (CDK2, CDKN2; p33(CDK2); NCBI Gene ID: 1017); cyclin dependent kinase 3 (CDK3; NCBI Gene ID: 1018); cyclin dependent kinase 4 (CDK4, CMM3; PSK-J3; NCBI Gene ID: 1019); cyclin dependent kinase 6 (CDK6, MCPH12; PLSTIRE; NCBI Gene ID: 1021); cyclin dependent kinase 7 (CDK7, CAK; CAK1; HCAK; MO15; STK1; CDKN7; p39MO15; NCBI Gene ID: 1022); cyclin dependent kinase 9 (CDK9, TAK; C-2k; CTK1; CDC2L4; PITALRE; NCBI Gene ID: 1025). Inhibitors of CDK 1, 2, 3, 4, 6, 7 and/or 9, include without limitation: abemaciclib, alvocidib (HMR-1275, flavopiridol), AT-7519, dinaciclib, ibrance, FLX-925, LEE001, palbociclib, ribociclib, rigosertib, selinexor, UCN-01, SY1365, CT-7001, SY-1365, G1T38, milciclib, trilaciclib, PF-06873600, AZD4573, and TG-02.

### Discoidin Domain Receptor (DDR) Inhibitors.

In various embodiments, the 5T4-targeting TriNKET® (anti-5T4×NKG2D×CD16 multispecific molecule), as described herein, is further combined with an inhibitor of discoidin domain receptor tyrosine kinase 1 (DDR1, CAK, CD167, DDR, EDDR1, HGK2, MCK10, NEP, NTRK4, PTK3, PTK3A, RTK6, TRKE; NCBI Gene ID: 780); and/or discoidin domain receptor tyrosine kinase 2 (DDR2, MIG20a, NTRKR3, TKT, TYRO10, WRCN; NCBI Gene ID: 4921). Examples of DDR inhibitors include without limitation, dasatinib and those disclosed in WO2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO2013/034933 (Imperial Innovations).

### Histone Deacetylase (HDAC) Inhibitors

In various embodiments, the 5T4-targeting TriNKET® (anti-5T4×NKG2D×CD16 multispecific molecule), as described herein, is further combined with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HIBI-8000), CUDC-907 (fimepinostat), entinostat, givinostat, mocetinostat, panobinostat, pracinostat, quisinostat (JNJ-26481585), resminostat, ricolinostat, SHP-141, valproic acid (VAL-001), vorinostat, tinostamustine, remetinostat, entinostat, romidepsin, and tucidinostat.

Indoleamine-pyrrole-2,3-dioxygenase (IDO1) Inhibitors

In various embodiments, the 5T4-targeting TriNKET® (anti-5T4×NKG2D×CD16 multispecific molecule), as described herein, is further combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Janus Kinase (JAK) Inhibitors

In various embodiments, the 5T4-targeting TriNKET® (anti-5T4×NKG2D×CD16 multispecific molecule), as described herein, is further combined with an inhibitor of Janus kinase 1 (JAK1, JAK1A, JAK1B, JTK3; NCBI Gene ID: 3716); Janus kinase 2 (JAK2, JTK10, THCYT3; NCBI Gene ID: 3717); and/or Janus kinase 3 (JAK3, JAK-3, JAK3_HUMAN, JAKL, L-JAK, LJAK; NCBI Gene ID: 3718). Examples of JAK inhibitors include without limitation, AT9283, AZD1480, baricitinib, BMS-911543, fedratinib, filgotinib (GLPG0634), gandotinib (LY2784544), INCB039110 (itacitinib), lestaurtinib, momelotinib (CYT0387), NS-018, pacritinib (SB1518), peficitinib (ASP015K), ruxolitinib, tofacitinib (formerly tasocitinib), INCB052793, and XL019.

### Matrix Metalloprotease (MMP) Inhibitors

In various embodiments, the 5T4-targeting TriNKET® (anti-5T4×NKG2D×CD16 multispecific molecule), as described herein, is further combined with an inhibitor of a matrix metallopeptidase (MMP), e.g., an inhibitor of MMP1 (NCBI Gene ID: 4312), MMP2 (NCBI Gene ID: 4313), MMP3 (NCBI Gene ID: 4314), MMP7 (NCBI Gene ID: 4316), MMP8 (NCBI Gene ID: 4317), MMP9 (NCBI Gene ID: 4318); MMP10 (NCBI Gene ID: 4319); MMP11 (NCBI Gene ID: 4320); MMP12 (NCBI Gene ID: 4321), MMP13 (NCBI Gene ID: 4322), MMP14 (NCBI Gene ID: 4323), MMP15 (NCBI Gene ID: 4324), MMP16 (NCBI Gene ID: 4325), MMP17 (NCBI Gene ID: 4326), MMP19 (NCBI Gene ID: 4327), MMP20 (NCBI Gene ID: 9313), MMP21 (NCBI Gene ID: 118856), MMP24 (NCBI Gene ID: 10893), MMP25 (NCBI Gene ID: 64386), MMP26 (NCBI Gene ID: 56547), MMP27 (NCBI Gene ID: 64066), and/or MMP28 (NCBI Gene ID: 79148). Examples of MMP9 inhibitors include without limitation, marimastat (BB-2516), cipemastat (Ro 32-3555), GS-5745 (andecaliximab), and those described in WO 2012/027721 (Gilead Biologics).

### RAS and RAS Pathway Inhibitors

In various embodiments, the 5T4-targeting TriNKET® (anti-5T4×NKG2D×CD16 multispecific molecule), as described herein, is further combined with an inhibitor of KRAS proto-oncogene, GTPase (KRAS; a.k.a., NS; NS3; CFC2; RALD; K-Ras; KRAS1; KRAS2; RASK2; KI-RAS; C-K-RAS; K-RAS2A; K-RAS2B; K-RAS4A; K-RAS4B; c-Ki-ras2; NCBI Gene ID: 3845); NRAS proto-oncogene, GTPase (NRAS; a.k.a., NS6; CMNS; NCMS; ALPS4; N-ras; NRAS1; NCBI Gene ID: 4893); HRas proto-oncogene, GTPase (HRAS; a.k.a., CTLO; KRAS; HAMSV; HRAS1; KRAS2; RASH1; RASK2; Ki-Ras; p21ras; C-H-RAS; c-K-ras; H-RASIDX; c-Ki-ras; C-BAS/HAS; aC-HA-RAS1; and NCBI Gene ID: 3265). The Ras inhibitors can inhibit Ras at either the polynucleotide (e.g., transcriptional inhibitor) or polypeptide (e.g., GTPase enzyme inhibitor) level. In some embodiments, the inhibitors target one or more proteins in the Ras pathway, e.g., inhibit one or more of EGFR, Ras, Raf (A-Raf, B-Raf, C-Raf), MEK (MEK1, MEK2), ERK, PI3K, AKT, and mTOR.

In various embodiments, the 5T4-targeting TriNKET® (anti-5T4×NKG2D×CD16 multispecific molecule), as described herein, is further combined with an inhibitor of KRAS. Examples of KRAS inhibitors include AMG-510, COTI-219, MRTX-1257, ARS-3248, ARS-853, WDB-178, BI-3406, BI-1701963, ARS-1620 (G12C), SML-8-73-1 (G12C), Compound 3144 (G12D), Kobe0065/2602 (Ras GTP), RT11, MRTX-849 (G12C), and K-Ras(G12D)-selective inhibitory peptides, including KRpep-2 (Ac-RRCPLYISYDPVCRR-NH2) (SEQ ID NO: 543) and KRpep-2d (Ac-RRRRCPLYISYDPVCRRRR-NH2) (SEQ ID NO: 544).

In various embodiments, the 5T4-targeting TriNKET® (anti-5T4×NKG2D×CD16 multispecific molecule), as described herein, is further combined with an inhibitor of KRAS mRNA. Illustrative KRAS mRNA inhibitors include anti-KRAS U1 adaptor, AZD-4785, siG12D-LODER™, and siG12D exosomes.

In various embodiments, the 5T4-targeting TriNKET® (anti-5T4×NKG2D×CD16 multispecific molecule), as described herein, is further combined with an inhibitor of MEK. Illustrative MEK inhibitors that can be co-administered include binimetinib, cobimetinib, PD-0325901, pimasertib, RG-7304, selumetinib, trametinib, and selumetinib.

In various embodiments, the 5T4-targeting TriNKET® (anti-5T4×NKG2D×CD16 multispecific molecule), as described herein, is further combined with an inhibitor of AKT. Illustrative AKT inhibitors that can be co-administered include RG7440, MK-2206, ipatasertib, afuresertib, AZD5363, and ARQ-092, capivasertib, triciribine, and ABTL-0812 (PI3K/Akt/mTOR).

In various embodiments, the 5T4-targeting TriNKET® (anti-5T4×NKG2D×CD16 multispecific molecule), as described herein, is further combined with an inhibitor of Raf. Illustrative Raf inhibitors that can be co-administered BGB-283 (Raf/EGFR), HM-95573, LXH-254, LY-3009120, RG7304, TAK-580, dabrafenib, vemurafenib, encorafenib (LGX818), PLX8394. RAF-265 (Raf/VEGFR), and ASN-003 (Raf/PI3K).

In various embodiments, the 5T4-targeting TriNKET® (anti-5T4×NKG2D×CD16 multispecific molecule), as described herein, is further combined with an inhibitor of ERK. Illustrative ERK inhibitors that can be co-administered include LTT-462, LY-3214996, MK-8353, ravoxertinib, GDC-0994, and ulixertinib.

In various embodiments, the 5T4-targeting TriNKET® (anti-5T4×NKG2D×CD16 multispecific molecule), as described herein, is further combined with an inhibitor of PI3K. Illustrative PI3K inhibitors that can be co-administered include idelalisib (Zydelig®), alpelisib, buparlisib, pictilisib, eganelisib (IPI-549). Illustrative PI3K/mTOR inhibitors that can be co-administered include dactolisib, omipalisib, voxtalisib, gedatolisib, GSK2141795, and RG6114.

In various embodiments, the 5T4-targeting TriNKET® (anti-5T4×NKG2D×CD16 multispecific molecule), as described herein, is further combined with an inhibitor of mTOR. Illustrative mTOR inhibitors that can be co-administered include as sapanisertib, vistusertib (AZD2014), ME-344, sirolimus (oral nano-amorphous formulation, cancer), and TYME-88 (mTOR/cytochrome P450 3A4).

In certain embodiments, Ras-driven cancers (e.g., NSCLC) having CDKN2A mutations can be inhibited by co-administration of the MEK inhibitor selumetinib and the CDK4/6 inhibitor palbociclib. See, e.g., Zhou, et al., Cancer Lett. 2017 Nov. 1; 408:130-137. Also, K-RAS and mutant N-RAS can be reduced by the irreversible ERBB1/2/4 inhibitor neratinib. See, e.g., Booth, et al., Cancer Biol Ther. 2018 Feb. 1; 19(2):132-137.

In various embodiments, the 5T4-targeting TriNKET® (anti-5T4×NKG2D×CD16 multispecific molecule), as described herein, is further combined with an inhibitor of RAS. Examples of RAS inhibitors include NEO-100 and rigosertib.

In various embodiments, the 5T4-targeting TriNKET® (anti-5T4×NKG2D×CD16 multispecific molecule), as described herein, is further combined with an antagonist of EGFR, such as AMG-595, necitumumab, ABBV-221, depatuxizumab mafodotin (ABT-414), tomuzotuximab, ABT-806, vectibix, modotuximab, and RM-1929.

In various embodiments, the 5T4-targeting TriNKET® (anti-5T4×NKG2D×CD16 multispecific molecule), as described herein, is further combined with an inhibitor of protein tyrosine phosphatase non-receptor type 11 (PTPN11; BPTP3, CFC, JMML, METCDS, NS1, PTP-1D, PTP2C, SH-PTP2, SH-PTP3, SHP2; NCBI Gene ID: 5781). Examples of SHP2 inhibitors include TNO155 (SHP-099), RMC-4550, JAB-3068, RMC-4630, SAR442720, and those described in WO2018172984 and WO2017211303.

In various embodiments, the 5T4-targeting TriNKET® (anti-5T4×NKG2D×CD16 multispecific molecule), as described herein, is further combined with an inhibitor of mitogen-activated protein kinase 7 (MAP2K7, JNKK2, MAPKK7, MEK, MEK 7, MKK7, PRKMK7, SAPKK-4, SAPKK4; NCBI Gene ID: 5609). Examples of MEK inhibitors include antroquinonol, binimetinib, CK-127, cobimetinib (GDC-0973, XL-518), MT-144, selumetinib (AZD6244), sorafenib, trametinib (GSK1120212), uprosertib+trametinib, PD-0325901, pimasertib, LTT462, AS703988, CC-90003, refametinib, TAK-733, CI-1040, and RG7421.

In various embodiments, the 5T4-targeting TriNKET® (anti-5T4×NKG2D×CD16 multispecific molecule), as described herein, is further combined with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p110-alpha; NCBI Gene ID: 5290); phosphatidylinositol-4,5-bisphosphate 3-kinase catalytic subunit beta (PIK3CB, P110BETA, PI3K, PI3KBETA, PIK3C1; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PI3K; Gene ID: 5494); and/or phosphatidylinositol-4,5-bisphosphate 3-kinase catalytic subunit delta (PIK3CD, APDS, IMD14, P110DELTA, PI3K, p110D, NCBI Gene ID: 5293). In some embodiments, the PI3K inhibitor is a pan-PI3K inhibitor. Examples of PI3K inhibitors include without limitation, ACP-319, AEZA-129, AMG-319, AS252424, AZD8186, BAY 1082439, BEZ235, bimiralisib (PQR309), buparlisib (BKM120), BYL719 (alpelisib), carboxyamidotriazole orotate (CTO), CH5132799, CLR-457, CLR-1401, copanlisib (BAY 80-6946), DS-7423, dactolisib, duvelisib (IPI-145), fimepinostat (CUDC-907), gedatolisib (PF-05212384), GDC-0032, GDC-0084 (RG7666), GDC-0077, pictilisib (GDC-0941), GDC-0980, GSK2636771, GSK2269577, GSK2141795, idelalisib (Zydelig®), INCB040093, INCB50465, IPI-443, IPI-549, KAR4141, LY294002, LY3023414, NERLYNX® (neratinib), nemiralisib (GSK2269557), omipalisib (GSK2126458, GSK458), OXY111A, panulisib (P7170, AK151761), PA799, perifosine (KRX-0401), Pilaralisib (SAR245408; XL147), puquitinib mesylate (XC-302), SAR260301, seletalisib (UCB-5857), serabelisib (INK-1117, MLN-1117, TAK-117), SF1126, sonolisib (PX-866), RG6114, RG7604, rigosertib sodium (ON-01910 sodium), RP5090, tenalisib (RP6530), RV-1729, SRX3177, taselisib, TG100115, umbralisib (TGR-1202), TGX221, voxtalisib (SAR245409), VS-5584, WX-037, X-339, X-414, XL499, XL756, wortmannin, ZSTK474, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).

### Spleen Tyrosine Kinase (SYK) Inhibitors

In various embodiments, the 5T4-targeting TriNKET® (anti-5T4×NKG2D×CD16 multispecific molecule), as described herein, is further combined with an inhibitor of spleen associated tyrosine kinase (SYK, p72-Syk, Gene ID: 6850). Examples of SYK inhibitors include without limitation, 6-(1H-indazol-6-yl)-N-(4-morpholinophenyl)imidazo[1,2-a]pyrazin-8-amine, BAY-61-3606, cerdulatinib (PRT-062607), entospletinib, fostamatinib (R788), HMPL-523, NVP-QAB 205 AA, R112, R343, tamatinib (R406), and those described in U.S. Pat. No. 8,450,321 (Gilead Connecticut) and those described in U.S. 2015/0175616.

### Tyrosine-kinase Inhibitors (TKIs)

In various embodiments, the 5T4-targeting TriNKET® (anti-5T4×NKG2D×CD16 multispecific molecule), as described herein, is further combined with a tyrosine kinase inhibitor (TKI). TKIs may target epidermal growth factor receptors (EGFRs) and receptors for fibroblast growth factor (FGF), platelet-derived growth factor (PDGF), and vascular endothelial growth factor (VEGF). Examples of TKIs include without limitation, axitinib, afatinib, ARQ-087 (derazantinib), asp5878, AZD3759, AZD4547, bosutinib, brigatinib, cabozantinib, cediranib, crenolanib, crizotinib, dacomitinib, dasatinib, dovitinib, E-6201, erdafitinib, erlotinib, gefitinib, gilteritinib (ASP-2215), FP-1039, HM61713, icotinib, imatinib, KX2-391 (Src), lapatinib, lestaurtinib, lenvatinib, midostaurin, nintedanib, ODM-203, olmutinib, osimertinib (AZD-9291), pazopanib, ponatinib, poziotinib, quizartinib, radotinib, rociletinib, sulfatinib (HMPL-012), sunitinib, famitinib L-malate, (MAC-4), tivoanib, TH-4000, MEDI-575 (anti-PDGFR antibody), and TAK-659.

Proteins of the present application can also be used as an adjunct to surgical removal of the primary lesion.

The amount of multispecific binding protein and additional therapeutic agent, and the relative timing of administration, may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multispecific binding protein may be administered during a time when the additional therapeutic agent(s) exerts its prophylactic or therapeutic effect, or vice versa.

### V. Pharmaceutical Compositions and Formulations

The present disclosure also describes pharmaceutical compositions and formulations that contain a therapeutically effective amount of a protein described herein. The composition or formulation can be formulated for use in a variety of drug delivery systems. One or more physiologically acceptable excipients or carriers can also be included in the composition for proper formulation. Suitable formulations for use in the present disclosure are found, for example, in Remington's Pharmaceutical Sciences, Mack Publishing Company, Philadelphia, Pa., 17th ed., 1985 and Steven Shire, “Monoclonal Antibodies: Meeting the Challenges in Manufacturing, Formulation, Delivery and Stability of Final Drug Product,” Woodhead Publishing; 1st edition (Apr. 24, 2015). For a brief review of methods for drug delivery, see, e.g., Langer (Science 249:1527-1533, 1990).

The intravenous drug delivery formulation described in the present application may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may be freeze-dried (lyophilized) and contained in about 12-60 vials. In certain embodiments, the formulation may be freeze-dried and 45 mg of the freeze-dried formulation may be contained in one vial. In certain embodiments, the about 40 mg to about 100 mg of freeze-dried formulation may be contained in one vial. In certain embodiments, freeze-dried formulation from 12, 27, or 45 vials are combined to obtain a therapeutic dose of the protein in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial.

The protein could exist in a liquid aqueous pharmaceutical formulation including a therapeutically effective amount of the protein in a buffered solution forming a formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, for example between 5 and 9 or between 6 and 8, and in certain embodiments, between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity. The pharmaceutical formulation includes one or more excipients and is maintained at a certain pH. The term “excipient,” as used herein, means any non-therapeutic agent added to the formulation to provide a desired physical or chemical property, for example, pH, osmolarity, viscosity, clarity, color, isotonicity, odor, sterility, stability, rate of dissolution or release, adsorption, or penetration.

The multi-specific binding proteins of the present disclosure can be formulated in a pharmaceutical formulation at various concentrations. In some embodiments, the pharmaceutical formulation includes greater than or equal to 1 mg/mL, greater than or equal to 10 mg/mL, greater than or equal to 20 mg/mL, greater than or equal to 30 mg/mL, greater than or equal to 40 mg/mL, greater than or equal to 50 mg/mL, greater than or equal to 60 mg/mL, greater than or equal to 70 mg/mL, greater than or equal to 80 mg/mL, greater than or equal to 90 mg/mL, greater than or equal to 100 mg/mL, greater than or equal to 125 mg/mL, greater than or equal to 150 mg/mL, greater than or equal to 175 mg/mL, or greater than or equal to 200 mg/mL of the multi-specific binding protein. In certain embodiments, the pharmaceutical formulation includes 1 mg/ml to 200 mg/ml, 2 mg/ml to 200 mg/ml, 5 mg/ml to 200 mg/ml, 7.5 mg/ml to 200 mg/ml, 10 mg/ml to 200 mg/ml, 12.5 mg/ml to 200 mg/ml, 15 mg/ml to 200 mg/ml, 20 mg/ml to 200 mg/ml, 25 mg/ml to 200 mg/ml, 50 mg/ml to 200 mg/ml, 75 mg/ml to 200 mg/ml, 100 mg/ml to 200 mg/ml, 125 mg/ml to 200 mg/ml, 150 mg/ml to 200 mg/ml, 175 mg/ml to 200 mg/ml, 1 mg/ml to 150 mg/ml, 2 mg/ml to 150 mg/ml, 5 mg/ml to 150 mg/ml, 7.5 mg/ml to 150 mg/ml, 10 mg/ml to 150 mg/ml, 12.5 mg/ml to 150 mg/ml, 15 mg/ml to 150 mg/ml, 20 mg/ml to 150 mg/ml, 25 mg/ml to 150 mg/ml, 50 mg/ml to 150 mg/ml, 75 mg/ml to 150 mg/ml, 100 mg/ml to 150 mg/ml, 125 mg/ml to 150 mg/ml, 1 mg/ml to 100 mg/ml, 2 mg/ml to 100 mg/ml, 5 mg/ml to 100 mg/ml, 7.5 mg/ml to 100 mg/ml, 10 mg/ml to 100 mg/ml, 12.5 mg/ml to 100 mg/ml, 15 mg/ml to 100 mg/ml, 20 mg/ml to 100 mg/ml, 25 mg/ml to 100 mg/ml, 50 mg/ml to 100 mg/ml, 75 mg/ml to 100 mg/ml, 1 mg/ml to 50 mg/ml, 2 mg/ml to 50 mg/ml, 5 mg/ml to 50 mg/ml, 7.5 mg/ml to 50 mg/ml, 10 mg/ml to 50 mg/ml, 12.5 mg/ml to 50 mg/ml, 15 mg/ml to 50 mg/ml, 20 mg/ml to 50 mg/ml, 25 mg/ml to 50 mg/ml, 1 mg/ml to 25 mg/ml, 2 mg/ml to 25 mg/ml, 5 mg/ml to 25 mg/ml, 7.5 mg/ml to 25 mg/ml, 10 mg/ml to 25 mg/ml, 12.5 mg/ml to 25 mg/ml, 15 mg/ml to 25 mg/ml, 20 mg/ml to 25 mg/ml, 1 mg/ml to 20 mg/ml, 2 mg/ml to 20 mg/ml, 5 mg/ml to 20 mg/ml, 7.5 mg/ml to 20 mg/ml, 10 mg/ml to 20 mg/ml, 12.5 mg/ml to 20 mg/ml, or 15 mg/ml to 20 mg/ml of the multi-specific binding protein. In some embodiments, the pharmaceutical formulation includes about 5 mg/ml, about 7.5 mg/ml, about 10 mg/ml. about 12.5 mg/ml, about 15 mg/ml, about 20 mg/ml, about 25 mg/ml, or about 50 mg/ml of the multi-specific binding protein. In certain embodiments, the pharmaceutical formulation includes about 15 mg/ml of the multi-specific binding protein.

In certain embodiments, the present application describes a formulation with an extended shelf life including a multispecific binding protein as described herein, in combination with mannitol, citric acid monohydrate, sodium citrate, disodium phosphate dihydrate, sodium dihydrogen phosphate dihydrate, sodium chloride, polysorbate 80, water, and sodium hydroxide.

### Excipients and pH

One or more excipients in the pharmaceutical formulation of the present disclosure may include a buffering agent. The term “buffering agent,” as used herein, refers to one or more components that when added to an aqueous solution is able to protect the solution against variations in pH when adding acid or alkali, or upon dilution with a solvent. In some embodiments, citrate, phosphate buffers, glycinate, carbonate, histidine buffers and the like can be used, in which case, sodium, potassium or ammonium ions can serve as counterions.

In certain embodiments, the buffer or buffer system includes at least one buffer that has a buffering range that overlaps fully or in part with the range of pH 6.0 to 7.0. In certain embodiments, the buffer has a pKa of about 6.0 to 7.0. In certain embodiments, the buffer includes citrate. In certain embodiments, the citrate is present at a concentration of 5 to 100 mM, 7.5 to 100 mM, 10 to 100 mM, 12.5 to 100 mM, 15 to 100 mM, 17.5 to 100 mM, 20 to 100 mM, 22.5 to 100 mM, 25 to 100 mM, 50 mM to 100 mM, 75 mM to 100 mM, 5 to 75 mM, 7.5 to 75 mM, 10 to 75 mM, 12.5 to 75 mM, 15 to 75 mM, 17.5 to 75 mM, 20 to 75 mM, 22.5 to 75 mM, 25 to 75 mM, 50 mM to 75 mM, 5 to 50 mM, 7.5 to 50 mM, 10 to 50 mM, 12.5 to 50 mM, 15 to 50 mM, 17.5 to 50 mM, 20 to 50 mM, 22.5 to 50 mM, 25 to 50 mM, 5 to 25 mM, 7.5 to 25 mM, 10 to 25 mM, 12.5 to 25 mM, 15 to 25 mM, 17.5 to 25 mM, 20 to 25 mM, 5 mM to 20 mM, 7.5 to 20 mM, 10 mM to 20 mM, 12.5 to 20 mM, 15 mM to 20 mM, 17.5 to 20 mM, 5 mM to 15 mM, 7.5 to 15 mM, or 10 mM to 15 mM. In certain embodiments, the citrate is present at a concentration of about 5 mM, about 7.5 mM, about 10 mM, about 12.5 mM, about 15 mM, about 17.5 mM about 20 mM, about 22.5 mM, about 25 mM, or about 50 mM. In certain embodiments, the citrate is present at a concentration of 20 mM.

In some embodiments, the citrate comprises sodium citrate, citric acid, or a combination thereof. In certain embodiments, the sodium citrate is present at a concentration of 15 mM to 25 mM, 17 mM to 21 mM, or 17.5 mM to 20.5 mM. In certain embodiments, the sodium citrate is present at a concentration of about 15 mM, about 16 mM, about 17 mM, about 18 mM, about 19 mM, about 20 mM, about 21 mM, about 22 mM, about 23 mM, about 24 mM, or about 25 mM. In certain embodiments, the sodium citrate is present at a concentration of about 18.9 mM. In certain embodiments, the citric acid is present at a concentration of 0.5 mM to 1.5 mM, 0.7 mM to 1.3 mM, or 1.0 mM to 1.2. In certain embodiments, the citric acid is present at a concentration of about 0.5 mM, about 0.6 mM, about 0.7 mM, about 0.8 mM, about 0.9 mM, about 1.0 mM, about 1.1 mM, about 1.2 mM, about 1.3 mM, about 1.4 mM, or about 1.5 mM. In certain embodiments, the citric acid is present in an amount of about 2.3 mg. In certain embodiments, the citrate is at pH 6.5.

The pharmaceutical formulation disclosed herein may have a pH of 6.0 to 7.0. For example, in certain embodiments, the pharmaceutical formulation has a pH of 6.1 to 7.0, 6.2 to 7.0, 6.3 to 7.0, 6.4 to 7.0, 6.5 to 7.0, 6.6 to 7.0, 6.7 to 7.0, 6.8 to 7.0, 6.9 to 7.0, 6.1 to 6.9, 6.2 to 6.9, 6.3 to 6.9, 6.4 to 6.9, 6.5 to 6.9, 6.6 to 6.9, 6.7 to 6.9, 6.8 to 6.9, 6.1 to 6.8, 6.2 to 6.8, 6.3 to 6.8, 6.4 to 6.8, 6.5 to 6.8, 6.6 to 6.8, 6.7 to 6.8, 6.1 to 6.7, 6.2 to 6.7, 6.3 to 6.7, 6.4 to 6.7, 6.5 to 6.7, 6.6 to 6.7, 6.1 to 6.6, 6.2 to 6.6, 6.3 to 6.6, 6.4 to 6.6, 6.5 to 6.6, 6.1 to 6.5, 6.2 to 6.5, 6.3 to 6.5, 6.4 to 6.5, 6.1 to 6.4, 6.2 to 6.4, 6.3 to 6.4, 6.1 to 6.3, 6.2 to 6.3, or 6.1 to 6.2. In certain embodiments, the pharmaceutical composition or pharmaceutical formulation has a pH of about 6.2, about 6.3, about 6.4, about 6.5, about 6.6, about 6.7, or about 6.8. In certain embodiments, the pharmaceutical formulation has a pH of about 6.5. Under the rules of scientific rounding, a pH greater than or equal to 5.95 and smaller than or equal to 6.05 is rounded as 6.0.

In certain embodiments, an aqueous formulation is prepared including a protein of the present disclosure in a pH-buffered solution. The buffer of the formulation may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86 mg/mL), and about 6.2 mg/mL of sodium chloride (e.g., 6.165 mg/mL). In certain embodiments, the buffer system includes about 1 to about 1.5 mg/mL of citric acid, about 0.25 to about 0.5 mg/mL of sodium citrate, about 1.25 to about 1.75 mg/mL of disodium phosphate dihydrate, about 0.7 to about 1.1 mg/mL of sodium dihydrogen phosphate dihydrate, and about 6.0 to about 6.4 mg/mL of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to about 15 mg/mL. In certain embodiments, the concentration of mannitol may be about 10 to about 14 mg/mL. In certain embodiments, the concentration of mannitol may be about 12 mg/mL. In certain embodiments, the polyol sorbitol may be included in the formulation.

A sugar, which acts as a tonicifier and may stabilize the antibody, may also be included in the formulation. The sugar is added to the formulation in an amount which may vary with respect to the desired isotonicity of the formulation. In certain embodiments, the aqueous formulation may be isotonic. In certain embodiments, the sugar which may be used in the formulation as a tonicity agent is sucrose. In certain embodiments, the sucrose concentration may be 150 mM to 200 mM. In certain embodiments, the concentration of sucrose may be 160 mM to 190 mM. In certain embodiments, the concentration of sucrose may be 170 mM to 180 mM. In certain embodiments, the concentration of sucrose may be 172.5 mM to 177.5 mM. In certain embodiments, the concentration of sucrose may be about 175.3 mM.

The one or more excipients in the pharmaceutical formulation disclosed herein further includes a detergent or surfactant. The term “surfactant,” as used herein, refers to a surface active molecule containing both a hydrophobic portion (e.g., alkyl chain) and a hydrophilic portion (e.g., carboxyl and carboxylate groups). Surfactants are useful in pharmaceutical formulations for reducing aggregation of a therapeutic protein. A detergent or surfactant may also be added to the formulation. Exemplary detergents include nonionic detergents such as polysorbates (e.g., polysorbates 20, 80, etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. Surfactants suitable for use in the pharmaceutical formulations are generally non-ionic surfactants and include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68, etc.). In certain embodiments, the surfactant is a polysorbate. In certain embodiments, the formulation may contain the surfactants polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

Surfactants suitable for use in the pharmaceutical formulations are generally non-ionic surfactants and include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl- or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.). In certain embodiments, the surfactant is a polysorbate. In certain embodiments, the surfactant is polysorbate 80.

The amount of a non-ionic surfactant contained within the pharmaceutical composition or pharmaceutical formulation of the present disclosure may vary depending on the specific properties desired of the formulation, as well as the particular circumstances and purposes for which the formulations are intended to be used. In certain embodiments, the pharmaceutical formulation includes 0.005% to 0.5% (w/v), 0.005% to 0.25% (w/v), 0.005% to 0.2% (w/v), 0.005% to 0.1% (w/v), 0.005% to 0.05% (w/v), 0.005% to 0.025% (w/v), 0.005% to 0.02% (w/v), 0.005% to 0.01% (w/v), 0.0075% to 0.5% (w/v), 0.0075% to 0.2% (w/v), 0.0075% to 0.25% (w/v), 0.0075% to 0.1% (w/v), 0.0075% to 0.05% (w/v), 0.0075% to 0.025% (w/v), 0.0075% to 0.02% (w/v), 0.0075% to 0.01% (w/v), 0.01% to 0.5% (w/v), 0.01% to 0.25% (w/v), 0.01% to 0.2% (w/v), 0.01% to 0.1% (w/v), 0.01% to 0.05% (w/v), 0.01% to 0.025% (w/v), or 0.01% to 0.02% (w/v) of the non-ionic surfactant (e.g., polysorbate 80). In certain embodiments, the pharmaceutical formulation includes 0.005% (w/v), 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.15% (w/v), 0.2% (w/v), 0.25% (w/v), 0.3% (w/v), 0.35% (w/v), 0.4% (w/v), 0.45% (w/v), or 0.5% (w/v) of polysorbate 80. In certain embodiments, the pharmaceutical formulation includes about 0.01% (w/v) polysorbate 80.

The amount of a non-ionic surfactant contained within the pharmaceutical composition or pharmaceutical formulation of the present disclosure may vary depending on the specific properties desired of the formulation, as well as the particular circumstances and purposes for which the formulations are intended to be used. In certain embodiments, the pharmaceutical formulation includes 0.005 mg/mL to 0.5 mg/mL, 0.005 mg/mL to 0.25 mg/mL, 0.005 mg/mL to 0.2 mg/mL, 0.005 mg/mL to 0.1 mg/mL, 0.005 mg/mL to 0.05 mg/mL, 0.005 mg/mL to 0.025 mg/mL, 0.005 mg/mL to 0.02 mg/mL, 0.005 mg/mL to 0.01 mg/mL, 0.0075 mg/mL to 0.5 mg/mL, 0.0075 mg/mL to 0.2 mg/mL, 0.0075 mg/mL to 0.25 mg/mL, 0.0075 mg/mL to 0.1 mg/mL, 0.0075 mg/mL to 0.05 mg/mL, 0.0075 mg/mL to 0.025 mg/mL, 0.0075 mg/mL to 0.02 mg/mL, 0.0075 mg/mL to 0.01 mg/mL, 0.01 mg/mL to 0.5 mg/mL, 0.01 mg/mL to 0.25 mg/mL, 0.01 mg/mL to 0.2 mg/mL, 0.01 mg/mL to 0.1 mg/mL, 0.01 mg/mL to 0.05 mg/mL, 0.01 mg/mL to 0.025 mg/mL, or 0.01 mg/mL to 0.02 mg/mL of the non-ionic surfactant (e.g., polysorbate 80). In certain embodiments, the pharmaceutical formulation includes 0.005 mg/mL, 0.01 mg/mL, 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.15 mg/mL, 0.2 mg/mL, 0.25 mg/mL, 0.3 mg/mL, 0.35 mg/mL, 0.4 mg/mL, 0.45 mg/mL, or 0.5 mg/mL of polysorbate 80. In certain embodiments, the pharmaceutical formulation includes about 0.1 mg/mL polysorbate 80.

The one or more excipients in the pharmaceutical formulation disclosed herein may further include a sugar or sugar alcohol. Sugars and sugar alcohols are useful in pharmaceutical formulations as thermal stabilizers. In certain embodiments, the pharmaceutical formulation includes a sugar alcohol, for example, a sugar alcohol derived from a monosaccharide (e.g., mannitol, sorbitol, or xylitol), a sugar alcohol derived from a disaccharide (e.g., lactitol or maltitol), or a sugar alcohol derived from an oligosaccharide. In certain embodiments, the pharmaceutical formulation includes a sugar, for example, a monosaccharide (glucose, xylose, or erythritol), a disaccharide (e.g., sucrose, trehalose, maltose, or galactose), or an oligosaccharide (e.g., stachyose). In specific embodiments, the pharmaceutical formulation includes sucrose.

The amount of the sugar or sugar alcohol contained within the formulation can vary depending on the specific circumstances and intended purposes for which the formulation is used. In certain embodiments, the pharmaceutical formulation includes the sugar or sugar alcohol at 2% to 10% (w/v), 3% to 10% (w/v), 4% to 10% (w/v), 5% to 10% (w/v), 6% to 10% (w/v), 7% to 10% (w/v), 8% to 10% (w/v), 9% to 10% (w/v), 2% to 9% (w/v), 3% to 9% (w/v), 4% to 9% (w/v), 5% to 9% (w/v), 6% to 9% (w/v), 7% to 9% (w/v), 8% to 9% (w/v), 2% to 8% (w/v), 3% to 8% (w/v), 4% to 8% (w/v), 5% to 8% (w/v), 6% to 8% (w/v), 7% to 8% (w/v), 2% to 7% (w/v), 3% to 7% (w/v), 4% to 7% (w/v), 5% to 7% (w/v), 6% to 7% (w/v), 2% to 6% (w/v), 3% to 6% (w/v), 4% to 6% (w/v), 5% to 6% (w/v), 2% to 5% (w/v), 3% to 5% (w/v), 4% to 5% (w/v), 2% to 4% (w/v), 3% to 4% (w/v), or 2% to 3% (w/v).

The amount of the sugar or sugar alcohol contained within the formulation can vary depending on the specific circumstances and intended purposes for which the formulation is used. In certain embodiments, the pharmaceutical formulation, includes sucrose at a concentration of 170 mM to 180 mM, 170.5 to 179.5, 171 mM to 179 mM, 171.5 to 178.5, 172 mM to 178 mM, 172.5 to 177.5, 173 mM to 177 mM, 173.5 to 176.5, 174 mM to 176 mM. In certain embodiments, the pharmaceutical formulation includes about 170 mM, about 172.5 mM, about 175 mM, about 177. mM 5, or about 180 mM sucrose. In certain embodiments, the pharmaceutical formulation includes about 175.2 mM sucrose.

In certain embodiments, the pharmaceutical formulation is isotonic. An “isotonic” formulation is one which has essentially the same osmotic pressure as human blood. Isotonic formulations generally have an osmotic pressure from about 250 to 350 mOsmol/kgH₂O. Isotonicity can be measured using a vapor pressure or ice-freezing type osmometer. In certain embodiments, the osmolarity of the pharmaceutical composition or pharmaceutical formulation is 250 to 350 mOsmol/kgH₂O. In certain embodiments, the osmolarity of the pharmaceutical composition or pharmaceutical formulation is 300 to 350 mOsmol/kgH₂O. Substances such as sugar, sugar alcohol, and NaCl can be included in the pharmaceutical formulation for desired osmolarity.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the multispecific binding proteins described in the present application may contain such bulking agents. A preservative reduces bacterial action and may, for example, facilitate the production of a multi-use (multiple-dose) formulation.

A preservative may be optionally added to the formulations herein to reduce bacterial action. The addition of a preservative may, for example, facilitate the production of a multi-use (multiple-dose) formulation.

In certain embodiments, the lyophilized drug product may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution.

In certain embodiments, the lyophilized drug product is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product is constituted to about 4.5 mL water for injection and diluted with 0.9% saline solution (sodium chloride solution).

### Exemplary Formulations

In some embodiments, the pharmaceutical formulation of the present disclosure includes a multi-specific binding protein, and one or more of: citrate; a sugar or sugar alcohol; and a polysorbate, at pH 6.0 to 7.0. In some embodiments, the pharmaceutical formulation of the present disclosure includes the multi-specific binding protein, citrate, a sugar or sugar alcohol, and a polysorbate, at pH 6.0 to 7.0. Also provided in the present disclosure are any one of the formulations above, further including one or more of: (a) citrate, (b) a sugar or sugar alcohol, and (c) a polysorbate.

In some embodiments, the present disclosure provides formulations consisting essentially of: (a) a multi-specific protein as described herein, (b) citrate, (c) a sugar or sugar alcohol, and (d) a polysorbate. The concentration of each of the components in that formulation can be any one of the concentrations or ranges as described in the present disclosure.

In some embodiments, the concentration of the multi-specific binding protein in the pharmaceutical formulation is 1 mg/mL to 125 mg/mL, 2 mg/mL to 100 mg/mL, 5 mg/mL to 50 mg/mL, 5 mg/mL to 20 mg/mL, or 10 mg/mL to 20 mg/mL. In some embodiments, the concentration of the multi-specific binding protein in the pharmaceutical formulation is about 15 mg/mL. In some embodiments, the formulation is diluted with a suitable diluent in the range of 1:0 to 1:10 prior to administration to a subject. In some embodiments, the concentration of citrate in the pharmaceutical formulation is 15 mM to 25 mM or 17.5 mM to 22.5 mM. In some embodiments, pharmaceutical formulations of the present disclosure contain about 20 mM citrate. In some embodiments, the formulation also contains a sugar. In some embodiments, the sugar is sucrose. In some embodiments, the concentration of sucrose is 170 mM to 180 mM or 172.5 mM to 177.5 mM. In some embodiments, the concentration of sucrose is about 175.2 mM. In some embodiments, the formulation includes a polysorbate and the polysorbate is 80. In some embodiments, the concentration of polysorbate 80 is 0.05 mg/m: to 0.15 mg/mL. In some embodiments, the concentration of polysorbate 80 is about 0.1 mg/mL. In some embodiments, the pH of the formulation is 6.2 to 6.8, or 6.4 to 6.6. In some embodiments, the pH of the formulation is about 6.5. In some embodiments, the formulation contains: (a) 5 mg/mL to 50 mg/mL of the multi-specific binding protein, (b) 15 mM to 25 mM citrate, (c) 170 mM to 180 mM sucrose, and (d) 0.05 mg/mL to 0.15 mg/mL polysorbate 80. In some embodiments, the formulation is at pH 6.2 to 6.8. In some embodiments, the formulation contains: (a) 10 mg/mL to 20 mg/mL of the multi-specific binding protein, (b) 17.5 mM to 22.5 mM citrate, (c) 172.5 to 177.5 mM sucrose, and 0.05 mg/mL to 0.15 mg/mL polysorbate 80. In some embodiments, the formulation is at pH 6.4 to 6.6. In some embodiments, the formulation contains: (a) 15 mg/mL of the multi-specific binding protein, (b) 20 mM citrate, (c) 175.2 mM citrate, and (d) 0.1 mg/mL polysorbate 80. In some embodiments, the formulation is at about pH 6.5.

In some embodiments, the concentration of the multi-specific binding protein in the pharmaceutical formulation is 1 mg/mL to 125 mg/mL, 2 mg/mL to 100 mg/mL, 5 mg/mL to 50 mg/mL, 5 mg/mL to 20 mg/mL, or 10 mg/mL to 20 mg/mL. In some embodiments, the concentration of the multi-specific binding protein in the pharmaceutical formulation is about 15 mg/mL. In some embodiments, the formulation is diluted with a suitable diluent in the range of 1:0 to 1:10 prior to administration to a subject. In some embodiments, the concentration of citrate in the pharmaceutical formulation is 15 mM to 25 mM or 17.5 mM to 22.5 mM. In some embodiments, pharmaceutical formulations of the present disclosure contain about 20 mM citrate. In some embodiments, the formulation also contains a sugar. In some embodiments, the sugar is sucrose. In some embodiments, the concentration of sucrose is 170 mM to 180 mM or 172.5 mM to 177.5 mM. In some embodiments, the concentration of sucrose is about 175.2 mM. In some embodiments, the formulation includes a polysorbate and the polysorbate is polysorbate 80. In some embodiments, the concentration of polysorbate 80 is 0.005% to 0.05% (w/v) or 0.0075% to 0.025% (w/v). In some embodiments, the concentration of polysorbate 80 is about 0.01% (w/v). In some embodiments, the pH of the formulation is 6.2 to 6.8, or 6.4 to 6.6. In some embodiments, the pH of the formulation is about 6.5. In some embodiments, the formulation contains: (a) 5 mg/mL to 50 mg/mL of the multi-specific binding protein, (b) 15 mM to 25 mM citrate, (c) 170 mM to 180 mM sucrose, and 0.005% to 0.05% (w/v) polysorbate 80. In some embodiments, the formulation is at pH 6.2 to 6.8. In some embodiments, the formulation contains: (a) 10 mg/mL to 20 mg/mL of the multi-specific binding protein, (b) 17.5 mM to 22.5 mM citrate, (c) 172.5 mM to 177.5 mM sucrose, and 0.0075% to 0.025% (w/v) polysorbate 80. In some embodiments, the formulation is at pH 6.4 to 6.6. In some embodiments, the formulation contains: (a) 15 mg/mL of the multi-specific binding protein, (b) 20 mM citrate, (c) 175.2 mM sucrose, and (d) 0.01% (w/v) polysorbate 80. In some embodiments, the formulation is at about pH 6.5.

In some embodiments, the pharmaceutical formulation of the present disclosure is contained in a vial. Accordingly, in some embodiments, provided herein are vials comprising: a) a multispecific binding protein as described herein; b) a buffer comprising citrate; c) sucrose; and d) a polysorbate. Such vials can comprise a pharmaceutical formulation having certain components and properties as described herein, including for example, wherein the pH of the pharmaceutical formulation being between 6.0 to 7.0, or about 6.5. In some embodiments, the vial comprises a certain amount of the multispecific binding protein, such as 100 mg to 200 mg of the multispecific binding protein or about 150 mg of the multispecific binding protein. In some embodiments, the vial comprises a certain amount of sodium citrate, such as 50 mg to 60 mg of sodium citrate, or about 55.5 mg of sodium citrate. In some embodiments, the vial comprises a certain amount of citric acid, such as 1.5 mg to 3 mg of citric acid, or about 2.3 mg of citric acid. In some embodiments, the vial comprises a certain amount of sucrose, such as 500 mg to 700 mg of sucrose, or about 600 mg of sucrose. In some embodiments, the vial comprises a certain amount of polysorbate 80, such as 0.5 mg to 1.5 mg of polysorbate 80, or about 1 mg of polysorbate 80. In some embodiments, the vial comprises a certain volume of the pharmaceutical composition, such as about 10 mL of the pharmaceutical formulation.

### Stability of the Multi-Specific Binding Protein

The pharmaceutical formulations disclosed herein exhibit high levels of stability. A pharmaceutical formulation is stable when the multi-specific binding protein within the formulation retains an acceptable physical property, chemical structure, and/or biological function after storage under defined conditions.

Stability can be measured by determining the percentage of the multi-specific binding protein in the formulation that remains in a native conformation after storage for a defined amount of time at a defined temperature. The percentage of a protein in a native conformation can be determined by, for example, size exclusion chromatography (e.g., size exclusion high performance liquid chromatography), where a protein in the native conformation is not aggregated (eluted in a high molecular weight fraction) or degraded (eluted in a low molecular weight fraction). In certain embodiments, more than 90%, 91%, 92%, 93%, 94%, 95%, 96%, 97%, 98%, or 99% of the multi-specific binding protein has native conformation, as determined by size-exclusion chromatography, after incubation at 30° C. for 28 days. In certain embodiments, more than 90%, 91%, 92%, 93%, 94%, 95%, 96%, 97%, 98%, or 99% of the multi-specific binding protein has native conformation, as determined by size-exclusion chromatography, after incubation at 40° C. for 28 days. In certain embodiments, more than 90%, 91%, 92%, 93%, 94%, 95%, 96%, 97%, 98%, or 99% of the multi-specific binding protein has native conformation, as determined by size-exclusion chromatography, after incubation at 50° C. for 28 days.

Stability can also be measured by determining the percentage of multi-specific binding protein present in a more acidic fraction (“acidic form”) relative to the main fraction of protein (“main charge form”). While not wishing to be bound by theory, deamidation of a protein may cause it to become more negatively charged and thus more acidic relative to the non-deamidated protein (see, e.g., Robinson, Protein Deamidation, (2002) PNAS 99(8):5283-88). The percentage of the acidic form of a protein can be determined by ion exchange chromatography (e.g., cation exchange high performance liquid chromatography) or imaged capillary isoelectric focusing (icIEF). In certain embodiments, at least 50%, 60%, 70%, 80%, or 90% of the multi-specific binding protein in the pharmaceutical formulation is in the main charge (neutral) form after incubation at 30° C. for 28 days. In certain embodiments, at least 50%, 60%, 70%, 80%, or 90% of the multi-specific binding protein in the pharmaceutical formulation is in the main charge (neutral) form after incubation at 40° C. for 28 days. In certain embodiments, at least 15%, 20%, 25%, 30%, 40%, 50%, 60%, 70%, 80%, or 90% of the multi-specific binding protein in the pharmaceutical formulation is in the main charge (neutral) form after incubation at 50° C. for 28 days.

In some embodiments, the pharmaceutical formulation is stable at room temperature for at least 1 month, at least 2 months, at least 3 months, at least 4 months, at least 5 months, at least 6 months, at least 7 months, at least 8 months, at least 9 months, at least 10 months, at least 11 months, at least 1 year, at least 1.5 years, or at least 2 years. In some embodiments, the pharmaceutical formulation is stable at −80° C. for at least 1 month, at least 2 months, at least 3 months, at least 4 months, at least 5 months, at least 6 months, at least 7 months, at least 8 months, at least 9 months, at least 10 months, at least 11 months at least 1 year, at least 1.5 years, or at least 2 years. In some embodiments, the pharmaceutical formulation is stable at −20° C. for at least 1 month, at least 2 months, at least 3 months, at least 4 months, at least 5 months, at least 6 months, at least 7 months, at least 8 months, at least 9 months, at least 10 months, at least 11 months, at least 1 year, at least 1.5 years, or at least 2 years. In some embodiments, the pharmaceutical formulation is stable at −5° C. for at least 1 month, at least 2 months, at least 3 months, at least 4 months, at least 5 months, at least 6 months, at least 7 months, at least 8 months, at least 9 months, at least 10 months, at least 11 months, at least 1 year, at least 1.5 years, or at least 2 years. In some embodiments, the pharmaceutical formulation is stable at refrigerated temperatures for at least 1 month, at least 2 months, at least 3 months, at least 4 months, at least 5 months, at least 6 months, at least 7 months, at least 8 months, at least 9 months, at least 10 months, at least 11 months, at least 1 year, at least 1.5 years, or at least 2 years.

As used herein, “room temperatures” are temperatures refers to ambient temperatures as understood by one of ordinary skill in the art. For example, room temperatures can include 10-30° C. or 20-25° C. For example, room temperature includes, but is not limited to, 10-30° C., 15-30° C., 20-30° C., 25-30° C., 10-25° C., 15-25° C., 20-25° C., 10-20° C., 15-20° C., or 10-15° C.

As used herein, “refrigerated temperatures” are temperatures at or below 4° C. For example, refrigerated temperatures include, but are not limited to, 2 to 4° C., 1 to 4° C., 0 to 4° C., −2 to 4° C., −3 to 4° C., −4 to 4° C., or −5 to 4° C.

### Dosage Forms

The pharmaceutical formulation can be prepared and stored as a liquid formulation or a lyophilized form. In certain embodiments, the pharmaceutical formulation is a liquid formulation for storage at 2-8° C. (e.g., 4° C.) or a frozen formulation for storage at-20° C. or lower. The sugar or sugar alcohol in the formulation is used as a lyoprotectant.

Prior to pharmaceutical use, the pharmaceutical formulation can be diluted in an appropriate buffer or diluent. In some embodiments, the pharmaceutical formulation can be diluted in 15 mM to 25 mM citrate (e.g., 15 mM to 25 mM, 16 mM to 25 mM, 17 mM to 25 mM, 18 mM to 25 mM, 19 mM to 25 mM, 20 mM to 25 mM, 21 mM to 25 mM, 22 mM to 25 mM, 23 mM to 25 mM, 24 mM to 25 mM, 15 mM to 24 mM, 15 mM to 23 mM, 15 mM to 22 mM, 15 mM to 23 mM, 15 mM to 22 mM, 15 mM to 21 mM, 15 mM to 20 mM, 15 mM to 19 mM, 15 mM to 18 mM, 15 mM to 17 mM, 15 mM to 16 mM, or about 20 mM); 170 mM to 180 mM sucrose (e.g., 170.5 to 179.5, 171 mM to 179 mM, 171.5 to 178.5, 172 mM to 178 mM, 172.5 to 177.5, 173 mM to 177 mM, 173.5 to 176.5, 174 mM to 176 mM), at pH 6.2 to 6.8. In some embodiments, the pharmaceutical formulation can be diluted in 20 mM citrate; about 175.2 mM sucrose; and 0.01% (w/v) polysorbate 80, at pH 6.5. In some embodiments, the pharmaceutical formulation can be diluted with a suitable diluent in the range of 1:0, 1:1, 1:1.5, 1:2, 1:3, 1:4, 1:5, 1:6, 1:7, 1:8, 1:8, 1:10, 1:15, 1:20, 1:25, 1:50, or 1:100 prior to administration to a subject.

In alternative embodiments, the pharmaceutical formulation can be diluted and/or reconstituted in an aqueous carrier that is suitable for the route of administration. Other exemplary carriers include sterile water for injection (SWFI), bacteriostatic water for injection (BWFI), a pH buffered solution (e.g., phosphate-buffered saline), sterile saline solution, Ringer's solution, or dextrose solution. For example, when prepared for intravenous administration, the pharmaceutical formulation can be diluted in a 0.9% sodium chloride (NaCl) solution. In certain embodiments, the diluted pharmaceutical formulation is isotonic and suitable for administration by intravenous infusion.

The pharmaceutical formulation includes the multi-specific binding protein at a concentration suitable for storage. In certain embodiments, the pharmaceutical formulation includes the multi-specific binding protein at a concentration of 1 mg/ml to 200 mg/ml, 2 mg/ml to 200 mg/ml, 5 mg/ml to 200 mg/ml, 7.5 mg/ml to 200 mg/ml, 10 mg/ml to 200 mg/ml, 12.5 mg/ml to 200 mg/ml, 15 mg/ml to 200 mg/ml, 20 mg/ml to 200 mg/ml, 25 mg/ml to 200 mg/ml, 50 mg/ml to 200 mg/ml, 75 mg/ml to 200 mg/ml, 100 mg/ml to 200 mg/ml, 125 mg/ml to 200 mg/ml, 150 mg/ml to 200 mg/ml, 175 mg/ml to 200 mg/ml, 1 mg/ml to 150 mg/ml, 2 mg/ml to 150 mg/ml, 5 mg/ml to 150 mg/ml, 7.5 mg/ml to 150 mg/ml, 10 mg/ml to 150 mg/ml, 12.5 mg/ml to 150 mg/ml, 15 mg/ml to 150 mg/ml, 20 mg/ml to 150 mg/ml, 25 mg/ml to 150 mg/ml, 50 mg/ml to 150 mg/ml, 75 mg/ml to 150 mg/ml, 100 mg/ml to 150 mg/ml, 125 mg/ml to 150 mg/ml, 1 mg/ml to 100 mg/ml, 2 mg/ml to 100 mg/ml, 5 mg/ml to 100 mg/ml, 7.5 mg/ml to 100 mg/ml, 10 mg/ml to 100 mg/ml, 12.5 mg/ml to 100 mg/ml, 15 mg/ml to 100 mg/ml, 20 mg/ml to 100 mg/ml, 25 mg/ml to 100 mg/ml, 50 mg/ml to 100 mg/ml, 75 mg/ml to 100 mg/ml, 1 mg/ml to 50 mg/ml, 2 mg/ml to 50 mg/ml, 5 mg/ml to 50 mg/ml, 7.5 mg/ml to 50 mg/ml, 10 mg/ml to 50 mg/ml, 12.5 mg/ml to 50 mg/ml, 15 mg/ml to 50 mg/ml, 20 mg/ml to 50 mg/ml, 25 mg/ml to 50 mg/ml, 1 mg/ml to 25 mg/ml, 2 mg/ml to 25 mg/ml, 5 mg/ml to 25 mg/ml, 7.5 mg/ml to 25 mg/ml, 10 mg/ml to 25 mg/ml, 12.5 mg/ml to 25 mg/ml, 15 mg/ml to 25 mg/ml, 20 mg/ml to 25 mg/ml, 1 mg/ml to 20 mg/ml, 2 mg/ml to 20 mg/ml, 5 mg/ml to 20 mg/ml, 7.5 mg/ml to 20 mg/ml, 10 mg/ml to 20 mg/ml, 12.5 mg/ml to 20 mg/ml, or 15 mg/ml to 20 mg/ml.

In certain embodiments, the pharmaceutical formulation is packaged in a container (e.g., a vial, bag, pen, or syringe). In certain embodiments, the formulation may be a lyophilized formulation or a liquid formulation. In certain embodiments, the amount of multi-specific binding protein in the container is suitable for administration as a single dose. In certain embodiments, the amount of multi-specific binding protein in the container is suitable for administration in multiple doses. In certain embodiments, the pharmaceutical formulation includes the multi-specific binding protein at an amount of 0.1 to 2000 mg. In certain embodiments, the pharmaceutical formulation includes the multi-specific binding protein at an amount of 1 to 2000 mg, 10 to 2000 mg, 20 to 2000 mg, 50 to 2000 mg, 100 to 2000 mg, 200 to 2000 mg, 500 to 2000 mg, 1000 to 2000 mg, 0.1 to 1000 mg, 1 to 1000 mg, 10 to 1000 mg, 20 to 1000 mg, 50 to 1000 mg, 100 to 1000 mg, 200 to 1000 mg, 500 to 1000 mg, 0.1 to 500 mg, 1 to 500 mg, 10 to 500 mg, 20 to 500 mg, 50 to 500 mg, 100 to 500 mg, 200 to 500 mg, 0.1 to 200 mg, 1 to 200 mg, 10 to 200 mg, 20 to 200 mg, 50 to 200 mg, 100 to 200 mg, 0.1 to 100 mg, 1 to 100 mg, 10 to 100 mg, 20 to 100 mg, 50 to 100 mg, 0.1 to 50 mg, 1 to 50 mg, 10 to 50 mg, 20 to 50 mg, 0.1 to 20 mg, 1 to 20 mg, 10 to 20 mg, 0.1 to 10 mg, 1 to 10 mg, or 0.1 to 1 mg. In certain embodiments, the pharmaceutical formulation includes the multi-specific binding protein at an amount of 0.1 mg, 1 mg, 2 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 150 mg, 200 mg, 250 mg, 300 mg, 400 mg, 500 mg, 600 mg, 700 mg, 800 mg, 900 mg, 1000 mg, 1500 mg, or 2000 mg. In certain embodiments, about 10 mL of the pharmaceutical formulation comprises about 150 mg of the multispecific binding protein.

Actual dosage levels of the active ingredients in the pharmaceutical compositions of multispecific binding proteins described in this application may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

In general, dosages based on body weight are from about 0.01 μg to about 100 mg per kg of body weight, such as about 0.01 μg to about 100 mg/kg of body weight, about 0.01 g to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 g to about 1 mg/kg of body weight, about 10 μg to about 100 μg/kg of body weight, about g to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

Doses may be given once or more times daily, weekly, monthly or yearly, or even once every 2 to 20 years. In some embodiments, the one or more administered doses are in the range of from about 50 μg/kg/week to about 20 mg/kg/week, e.g., from about 100 g/kg/week to about 12 mg/kg/week, e.g., from about 100 μg/kg/week to about 6 mg/kg/week, e.g., from about 100 μg/kg/week to about 3 mg/kg/week. In some embodiments, one or more doses of about 50 μg/kg/week are administered. In some embodiments, one or more doses of about 100 μg/kg/week are administered. In some embodiments, one or more doses of about 300 μg/kg/week are administered. In some embodiments, one or more doses of about 1 mg/kg/week are administered. In some embodiments, one or more doses of about 3 mg/kg/week are administered. In some embodiments, one or more doses of about 6 mg/kg/week are administered. In some embodiments, one or more doses of about 12 mg/kg/week are administered. In some embodiments, one or more doses of about 20 mg/kg/week are administered.

Persons of ordinary skill in the art can easily estimate repetition rates for dosing based on measured residence times and concentrations of the targetable construct or complex in bodily fluids or tissues. Administration of the multispecific binding proteins described in the present application could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

Administration of the pharmaceutical formulations described herein can be intravenous, intraarterial, intraperitoneal, intramuscular, subcutaneous, intrapleural, intrathecal, intracavitary, by perfusion through a catheter or by direct intralesional injection.

In embodiments, a multispecific binding protein as described in the present application is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments vials may be filled with 61.2 mL of the protein product solution in order to allow an extractable volume of 60 mL. In certain embodiments, the liquid formulation may be diluted with 0.9% saline solution.

In certain embodiments, the liquid formulation as described in this application may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be a disaccharide, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 Dalton mass decrease of the parent peptide. The subsequent hydrolysis results in an 18 Dalton mass increase. Isolation of the succinimide intermediate is difficult due to instability under aqueous conditions. As such, deamidation is typically detectable as 1 Dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation as described in this application may be preserved under conditions of pH and humidity to prevent deamination of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations described herein to reduce bacterial action. The addition of a preservative may, for example, facilitate the production of a multi-use (multiple-dose) formulation.

Intravenous (IV) formulations may be an administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A multispecific binding protein as described in the present application could exist in a lyophilized formulation including the proteins and a lyoprotectant. The lyoprotectant may be a sugar, e.g., a disaccharide. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing a protein of the present disclosure may be adjusted between 6 to 8. In certain embodiments, the pH range for the lyophilized drug product may be from 7 to 8.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

### Kits

The formulation of a multispecific binding protein described herein is prepared as a lyophilized formulation or a liquid formulation. For preparing the lyophilized formulation, freeze-dried a multispecific binding protein is sterilized and stored in single-use glass vials. Several such glass vials are then packaged in a kit for delivering a dose to a subject diagnosed with a cancer or a tumor.

In one aspect, the present application provides a kit including one or more vessels collectively including a formulation of about 10 mg, about 20 mg, about 30 mg, about 40 mg, about 50 mg, about 60 mg, about 70 mg, about 80 mg, about 90 mg, about 100 mg, about 120 mg, about 130 mg, about 140 mg, about 150 mg, about 200 mg, about 300 mg, about 400 mg, about 500 mg, about 600 mg, about 700 mg, about 800 mg, about 900 mg, or about 1 g of a multispecific binding protein. In certain embodiments, the present disclosure provides a kit including one or more vessels collectively including a formulation of about 150 mg of a multispecific binding protein.

In certain embodiments, the formulation is prepared and packaged as a liquid formulation and stored as about as about 100 mg/vial to 200 mg/vial (e.g., 110 mg/vial to 190 mg/vial, 120 mg/vial to 180 mg/vial, 130 mg/vial to 170 mg/vial, or 140 mg/vial to 160 mg/vial). In certain embodiments, the formulation is stored as about as about 100 mg/vial, about 110 mg/vial, about 120 mg/vial, about 130 mg/vial, about 140 mg/vial, about 150 mg/vial, about 160 mg/vial, about 170 mg/vial, about 180 mg/vial, about 190 mg/vial, or about 200 mg/vial. In certain embodiments, the formulation is a liquid formulation and stored as about 150 mg/vial.

In certain embodiments, the formulation is prepared and packaged as a lyophilized formulation and stored as about as about 100 mg/vial to 200 mg/vial (e.g., 110 mg/vial to 190 mg/vial, 120 mg/vial to 180 mg/vial, 130 mg/vial to 170 mg/vial, or 140 mg/vial to 160 mg/vial). In certain embodiments, the formulation is stored as about as about 100 mg/vial, about 110 mg/vial, about 120 mg/vial, about 130 mg/vial, about 140 mg/vial, about 150 mg/vial, about 160 mg/vial, about 170 mg/vial, about 180 mg/vial, about 190 mg/vial, or about 200 mg/vial. In certain embodiments, the formulation is a liquid formulation and stored as about 150 mg/vial.

In certain embodiments, the formulation in the vessels may be a lyophilized formulation. In certain embodiments, the formulation in the vessels may be a liquid formulation.

In certain embodiments, the formulation may be packed in kits containing a suitable number of vials. The information on the medication may be included, which are in accordance with approved submission documents. The kit may be shipped in transport cool containers (2° C. to 8° C.) that are monitored with temperature control devices.

The formulation may be stored at 2° C. to 8° C. until use. The vials of the formulations may be sterile and nonpyrogenic, and may not contain bacteriostatic preservatives.

The description above provides multiple aspects and embodiments of the multispecific binding proteins described in the application. The patent application specifically contemplates all combinations and permutations of the aspects and embodiments. The use of any and all examples, or exemplary language herein, for example, “such as” or “including,” is intended merely to illustrate better the multispecific binding proteins described in the present application, and does not pose a limitation on the scope of the disclosure, unless so expressly stated. No language in the specification should be construed as indicating any non-claimed element as essential to the practice of the multispecific binding proteins described in the present application.

### EXAMPLES

The following examples are merely illustrative and are not intended to limit the scope or content of the multispecific binding proteins described in the present application in any way.

### Example 1—Assessment of TriNKET® Binding to Cell Expressed Human 5T4

Generation and Characterization of 5T4 Binding mAbs

This Example describes newly identified binders of 5T4 from an antibody discovery campaign. One binder, AB1002-scFv, was chosen for further development and is characterized below.

### 5T4-Antibody Generation

5T4-specific antibodies were selected starting with 131 hybridoma antibodies that bind to recombinant human 5T4-His. Of these, 113 were found to bind to human 5T4 (h5T4) on the cell surface. From these, 78 clones showed binding to human 5T4 by surface plasmon resonance (SPR). 62 clones showed binding to rhesus 5T4 (r5T4) by SPR. 15 clones bound to h5T4 & r5T4 equally well and satisfied affinity criteria. These studies identified murine 10F10 as a binder displaying properties appropriate for a biologics drug candidate. Additional murine binders 11F09 and 08E06 were also identified as having desirable characteristics, and murine 05H04 was identified as having a subset of desirable characteristics (though lacking in binding to cynomolgus 5T4).

### Affinity Maturation of Clone 08E06

To search for variants of clone 08E06 with improved binding affinities, a yeast display affinity maturation library was created by mutating the CDRH3 residue (GGYLWFAY (SEQ ID NO:188). To enrich for scFvs that have higher affinity towards human 5T4 (h5T4), two rounds of selection were carried out with biotinylated h5T4-R-hFc-His at 1 nM. Affinities between the parental clone 08E06 and representative individual library clones were compared, and multiple rounds of FACS were performed.

Outcomes from the CDRH3 focused affinity maturation studies demonstrated an improvement in affinity, and further improvement was highly desirable. Thus, the CDRH1 and CDRH2 sequences were selected for affinity maturation (CDRH1: GYTFTSY (SEQ ID NO:186) and CDRH2: DSSDSK (SEQ ID NO: 187)) using the matured CDRH3 backbone. The goal was to engineer and select binders with improved affinity over the parental clone (08E06 scFv) or the CDRH3 optimized variants described above. This created a library with a randomized CDRH1 and CDRH2 while retaining an optimized CDRH3. Multiple rounds of FACS were performed to enrich for high affinity binders of h5T4. 53 affinity matured clones were obtained in total from these processes. CDR sequences of select resultant affinity matured variants of clone 08E06 are shown in Table 12.

TABLE 12
Affinity Matured Variants of clone 08E06

| Clone | CDRH1 | CDRH2 | CDRH3 |
|---|---|---|---|
| 08E06 | GYTFTSY (SEQ | DSSDSK (SEQ | GGYLWFAY (SEQ |
| | ID NO: 186) | ID NO: 187) | ID NO: 188) |
| AB1558 | GYTFGSY (SEQ | DASTEK (SEQ | GGYLWFQY (SEQ |
| | ID NO: 203) | ID NO: 204) | ID NO: 205) |
| AB1566 | GYLFTSY (SEQ | SVSDAK (SEQ | GGYLWFKY (SEQ |
| | ID NO: 206) | ID NO: 207) | ID NO: 208) |
| AB1554 | GYTFGSY (SEQ | DARSAK (SEQ | GGYLWFKY (SEQ |
| | ID NO: 203) | ID NO: 209) | ID NO: 208) |
| AB1574 | GYRFTSY (SEQ | DASSAK (SEQ | GGYLWFKY (SEQ |
| | NQ:210) | ID NO: 211) | ID NO: 208) |
| AB1579 | GYGFTSY (SEQ | DARTAK (SEQ | GGYLWYAY (SEQ |
| | ID NO: 212) | ID NO: 213) | ID NO: 214) |
| AB1540 | GYTFTSY (SEQ | DASDAK (SEQ | GGYLWYHY (SEQ |
| | ID NO: 186) | ID NO: 215) | ID NO: 216) |
| AB1541 | GYTFTSY (SEQ | DASDAK (SEQ | GGYLWYSY (SEQ |
| | ID NO: 186) | ID NO: 215) | ID NO: 217) |
| AB1552 | GYTFTSY (SEQ | DASDAK (SEQ | GGYLWYAY (SEQ |
| | ID NO: 186) | ID NO: 215) | ID NO: 214) |
| AB1565 | GYSFTSY (SEQ | DASDAK (SEQ | GGYLWFKY (SEQ |
| | ID NO: 218) | ID NO: 215) | ID NO: 208) |
| AB1534 | GYTFTSY (SEQ | DASDAK (SEQ | GGYLWFKY (SEQ |
| | ID NO: 186) | ID NO: 215) | ID NO: 208) |
| AB1573 | GYGFTSY (SEQ | DARTAK (SEQ | GGHLWYAY (SEQ |
| | ID NO: 212) | ID NO: 213) | ID NO: 219) |

### Humanization and Sequence Liability Assessment

Clones 10F10 and 11F09 were humanized into multiple framework sequences; the sequences of these humanized clones are provided in Table 2 above. Amino acids that could negatively impact protein expression, stability, or immunogenicity, were replaced with alternatives which may address these issues. Sequences of these putative liability-corrected clones are also provided in Table 2, above.

AB1002 (a humanized variant of murine 10F10, with VH T62S correction to replace rare residue T62), was ultimately selected for further development.

### Epitope Mapping

Binding of 5T4 binders in relation to reference 5T4 antibodies was performed to determine binding epitope. The epitopes of murine 10F10 and murine 11F09 were mapped onto 5T4 in the leucine-rich repeat 1 (LRR1) domain. Furthermore, the epitope of murine 08E06 was mapped onto 5T4 in the leucine-rich repeat 2 (LRR2) domain. See, e.g., Zhao, et al., Structure (2014) 22(4):612-20.

### Surface Plasmon Resonance (SPR)

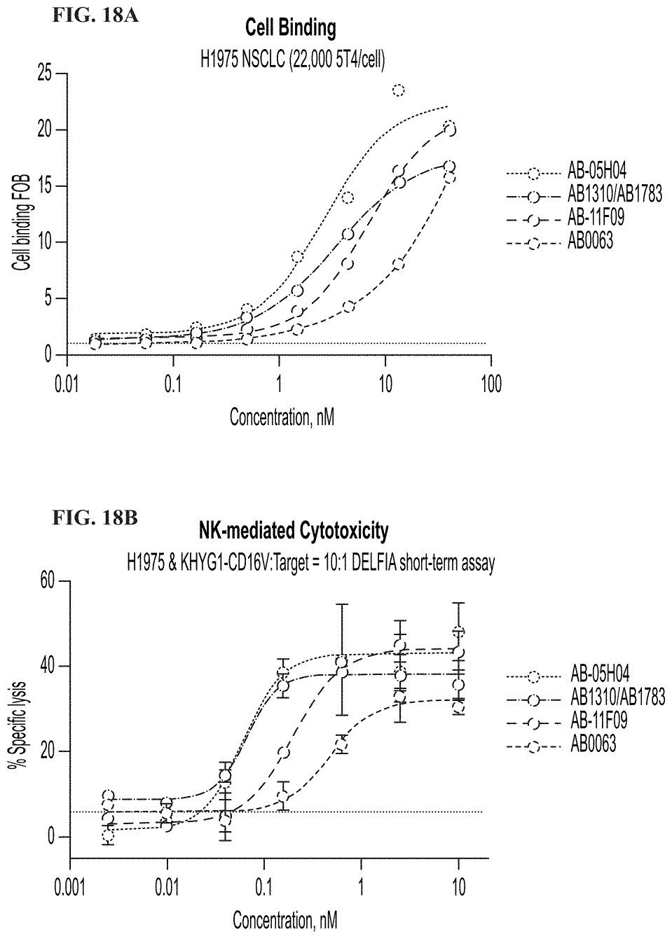

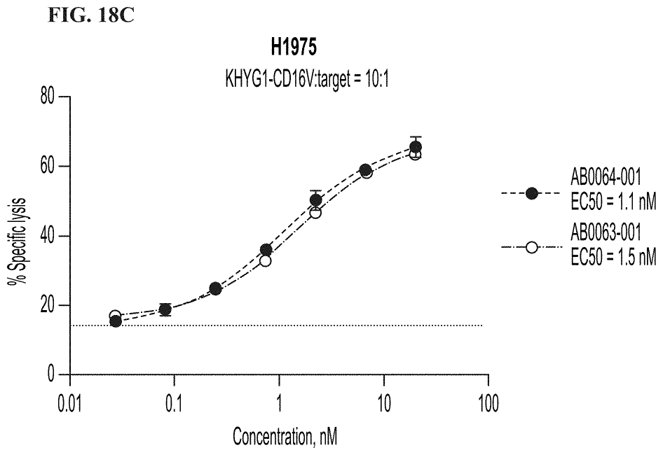

10F10 (AB1002 scFv) was converted into a multispecific binding protein comprising the 5T4-scFv, and two non-5T4 binders, to yield AB1310/AB1783-TriNKET®. Additionally, 08E06 was converted similarly to two multispecific binding proteins (AB0063-TriNKET® (VH-VL) and AB0064-TriNKET® (VL-VH)). Clones 05H04 and 11F09, were similarly used to produce multispecific binding proteins. The abilities of these multispecific binding protein to bind to 5T4-expressing cells is shown in FIG. 18A. Additionally, the abilities of these multispecific binding proteins to induce NK cell-mediated cytotoxicity of 5T4-expressing cells is shown in FIG. 18B and FIG. 18C. Results of cell binding and cytotoxicity analyses of the multispecific binding proteins are summarized in Table 13.

TABLE 13
Cell Binding and Cytotoxicity Analysis of
Candidate Multispecific Binding Proteins

| Clone | Cell Binding | | Cytotoxicity | |
|---|---|---|---|---|
| (F0) | EC50 (nM) | Max (FOB) | EC50 (nM) | Max. lysis (%) |
| 5H04* | 2.7 | 22.6 | 0.054 | 46 |
| 10F10 | 3.4 | 17.9 | 0.059 | 40 |
| 11F09 | 6.8 | 21.6 | 0.21 | 47 |
| 8E06 | >12 | — | 0.51 | 35 |

The binding affinities of AB1310/AB1783-TriNKET® to 5T4 were measured by surface plasmon resonance (SPR). Briefly, SPR was performed using a Biacore 8K instrument at physiological temperature of 37° C. Briefly, human Fc specific antibodies were covalently immobilized at a density of about 8000-10000 resonance units (RU) on carboxy methyl dextran matrix of a CM5 biosensor chip to create an anti-hFc IgG chip. Samples were injected on the anti-hFc IgG chip at a flow rate of 10 μL/min for 60 seconds. Protein was serially diluted (300 nM-0.14 nM) in three-fold dilutions with running buffer and injected at a flow rate of 30 μl/min over the captured test articles. Association was monitored for 240-300 seconds, and dissociation was monitored for 300-900 seconds. Surfaces were regenerated between cycles with three pulses of 10 mM glycine-HCl (pH 1.7) injected for 20 seconds at 100 μL/min.

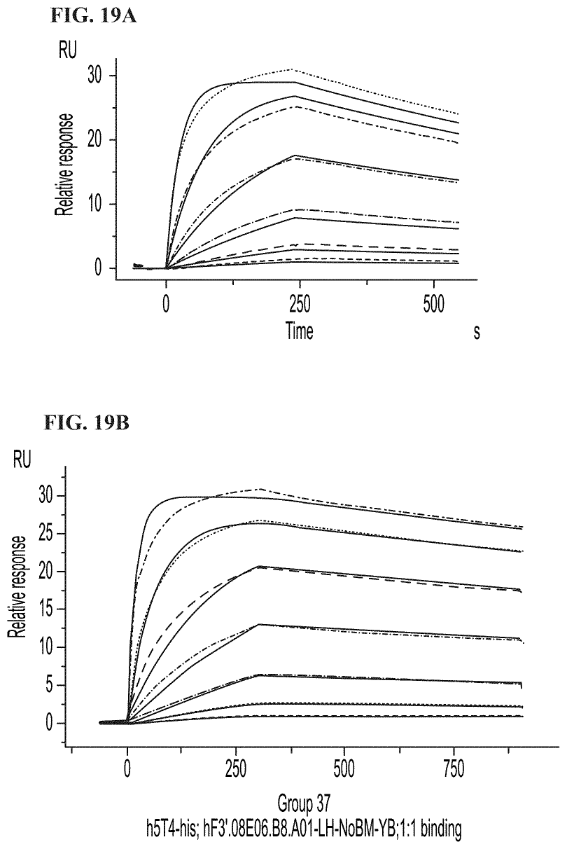

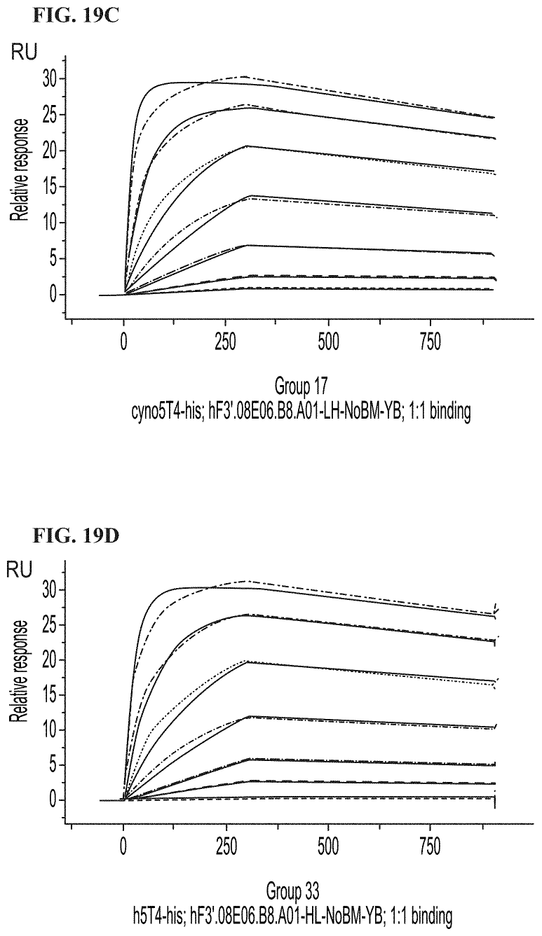

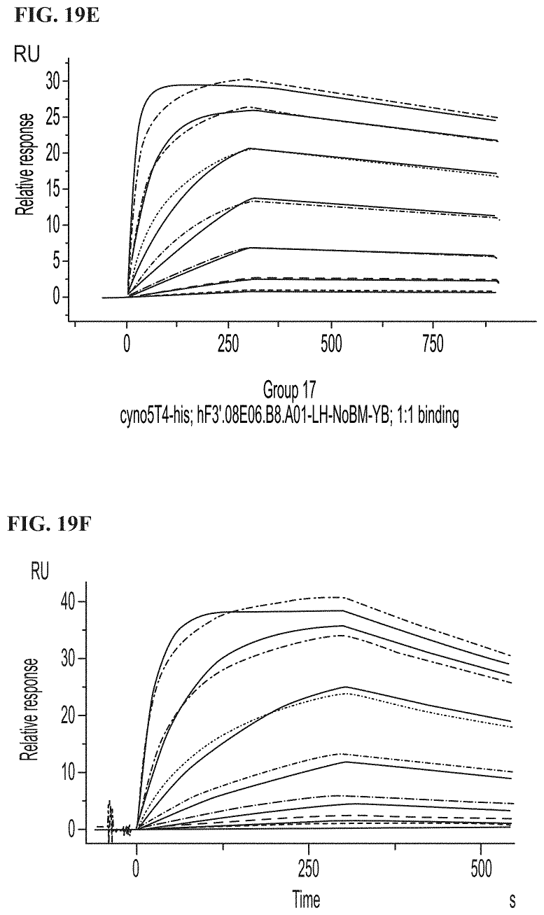

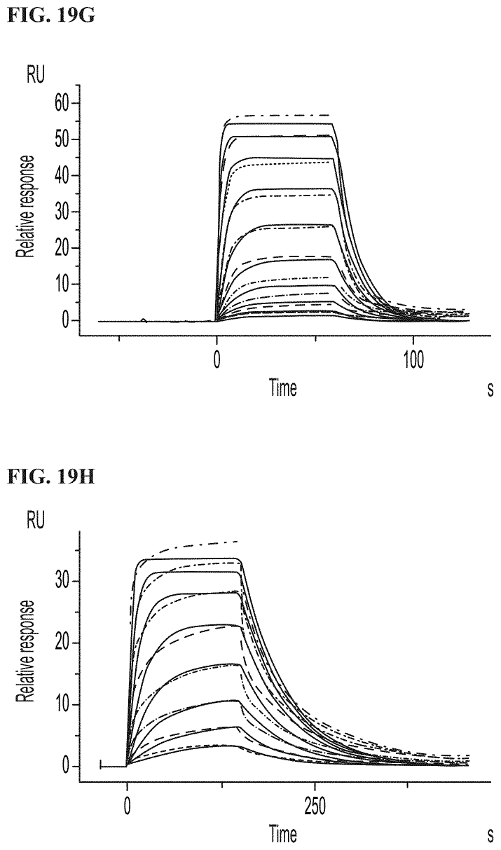

SPR analysis showed that AB1310/AB1783-TriNKET®, AB0063-TriNKET®, and AB0064-TriNKET® had high affinity for 5T4 (FIGS. 19A-19E). This binding affinity was maintained at a lower pH (6.0), which is representative of the tumor microenvironment (FIG. 19F). AB1310/AB1783-TriNKET® was also assayed for its binding to NKG2D and CD16A (FIGS. 19G and 19H). Details of SPR analysis of 5T4-TriNKET® binding to 5T4 are shown in Tables 14 and 15, and comparative binding to human and cynomolgus monkey 5T4 for AB1310/AB1783-TriNKET® is shown in Table 16.

TABLE 14
Binding affinities of AB1310/AB1783, AB0063, and
AB0064 TriNKETs ® to human 5T4

| | | | | Kinetics fit |
|---|---|---|---|---|
| Molecule | pH | kₐ (M⁻¹s⁻¹) | k_{d} (s⁻¹) | K_{D} (nM) |
| AB1310/ | 7.4 | (1.3 ± 0.0) × 10⁵ | (7.7 ± 0.3) × 10⁻⁴ | 6.1 ± 0.2 |
| AB1783- | 6.0 | (1.2 ± 0.1) × 10⁵ | (1.2 ± 0.0) × 10⁻³ | 9.4 ± 0.6 |
| TriNKET ® | | | | |
| AB0064- | 7.4 | 1.56 × 10⁵ | 2.49 × 10⁻⁴ | 1.6 |
| TriNKET ® | | | | |
| AB0063- | 7.4 | 1.28 × 10⁵ | 2.42 × 10⁻⁴ | 1.9 |
| TriNKET ® | | | | |

TABLE 15
Binding affinities of AB1310/AB1783-TriNKET ® to
human 5T4, NKG2D, and CD16a

| | | | Kinetics fit |
|---|---|---|---|
| Target | kₐ (M⁻¹s⁻¹) | k_{d} (s⁻¹) | K_{D} (nM) |
| Human 5T4 | (1.3 ± 0.0) × 10⁵ | (7.7 ± 0.3) × 10⁻⁴ | 6.1 ± 0.2 |
| Human NKG2D | (2.4 ± 0.1) × 10⁵ | (8.8 ± 0.0) × 10⁻² | 369.5 ± 9.2 |
| Human CD16aV | (1.5 ± 0.0) × 10⁵ | (1.7 ± 0.1) × 10⁻² | 111.8 ± 4.0 |

TABLE 16
Binding affinity of AB1310/AB1783-TriNKET ® in SPR assay

| Species | K_{D} 5T4 (nM) |
|---|---|
| Human | 6.2 ± 0.4 |
| Cynomolgus monkey | 8.3 ± 2.2 |

### Assessment of Specificity and Cell-Induced Cytotoxictiy

Studies were conducted to assess the binding affinity of the 5T4-targeting TriNKET® to 5T4 expressed on the cell surface. The KYSE-30 and H292 human cancer cell lines, derived from esophageal squamous cell carcinoma and non-small cell lung carcinoma, respectively, were used. KYSE-30 cells express higher levels of surface 5T4 than is found on H292 cells. Briefly, tumor cells were incubated with 5T4-TriNKET® or 5T4-mAb at 4° C. for 1 hour. After incubation, binding patterns of the TriNKET® and mAb to 5T4+ cells were detected using a fluorophore conjugated anti-human IgG secondary antibody. Cells were analyzed by flow cytometry and fold MFI over secondary-only controls reported. Additionally, the same protocol was used to assess binding of 5T4-TriNKET® to 5T4-expressing primary lung cancer-associated fibroblasts.

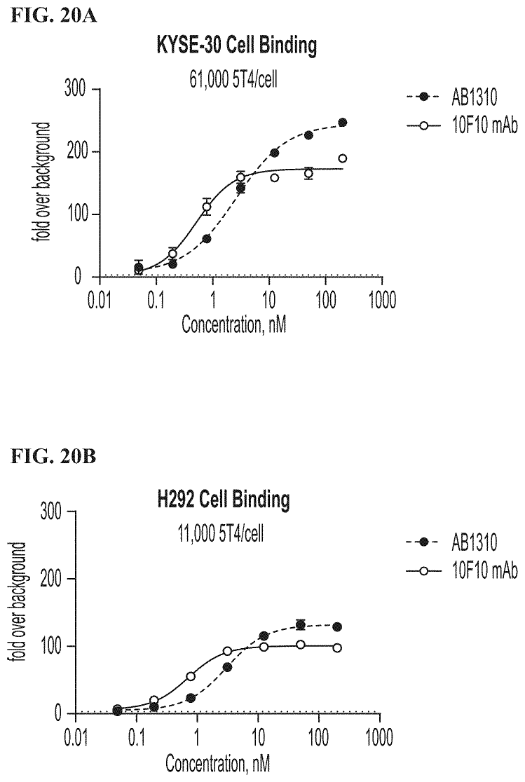

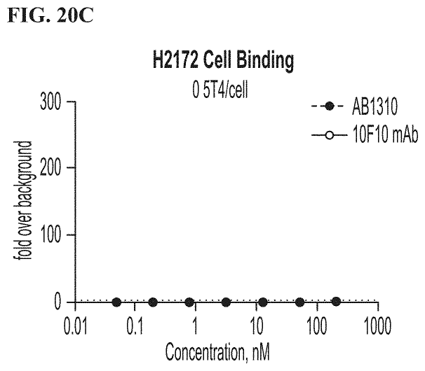

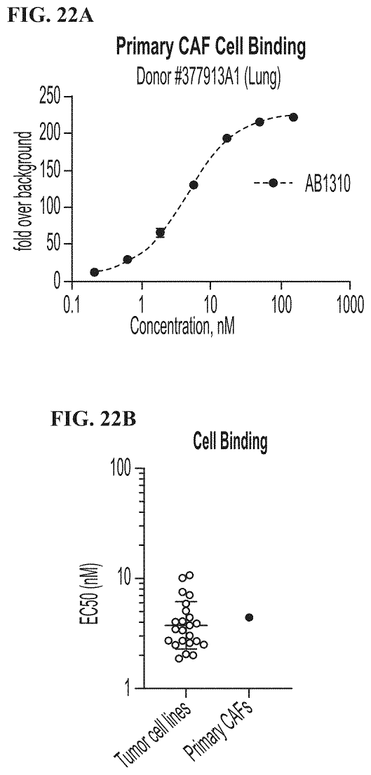

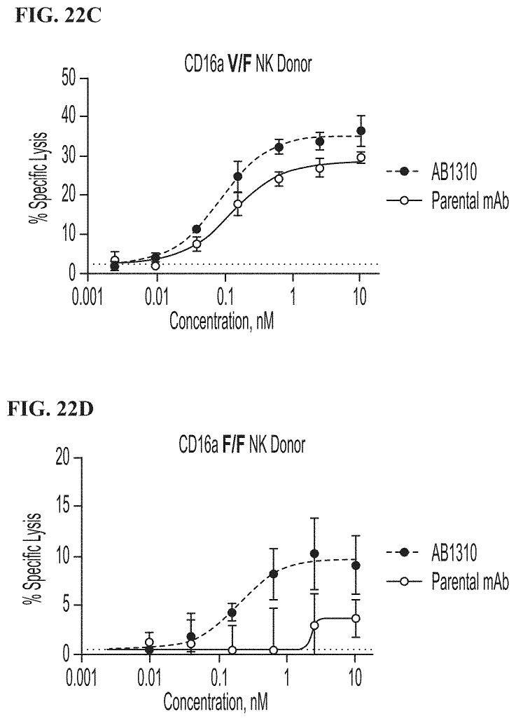

FIG. 20A, FIG. 20B, FIG. 22A and FIG. 22B show binding to 5T4-positive target cells after incubation with 5T4-TriNKET® or 5T4-mAb. TriNKET® bound to a higher magnitude on KYSE-30 cells, which express greater levels of surface 5T4 than H292 cells do. The 5T4-targeting TriNKET® bound the cells with single-digit nM concentrations and with higher maximum binding than 5T4-mAb. In addition, robust binding of 5T4-TriNKET® was observed to primary cancer-associated fibroblasts. By contrast, neither AB1310/AB1783-TriNKET® nor the parental monoclonal antibody (10F10), showed binding to the 5T4⁻ cell line H2712 (FIG. 20C).

Studies were conducted to assess the ability of the 5T4-targeting TriNKETs® to mediate cytotoxicity of immune effector cells against 5T4-expressing cancer cells. Briefly, peripheral blood mononuclear cells (PBMCs) were isolated from human peripheral blood buffy coats using density gradient centrifugation. Isolated PBMCs were washed and prepared for NK or CD8+ cell isolation. NK cells were isolated using a negative selection technique with magnetic beads, and the purity of isolated NK cells was typically >90% CD3-CD56+. Isolated NK cells were rested overnight in culture media without supplemental cytokines and used the following day in cytotoxicity assays. CD8+ were isolated using a negative selection technique with magnetic beads, and the purity of CD8+ cells was typically >90% CD3+CD56−CD8+. Isolated CD8+ T cells were incubated in media with IL-15 for 10 days for expansion.

For the cytotoxicity assays, human cancer cell lines or primary cancer-associated fibroblasts expressing 5T4 were harvested from culture, cells were washed with HBS, and resuspended in growth media at 10⁶/mL for labeling with BATDA reagent (Perkin Elmer ADO 116). Manufacturer instructions were followed for labeling of the target cells. After labeling, cells were washed 3× with HBS, and were resuspended at 0.5-1.0×10⁵/mL in culture media. To prepare the background wells, an aliquot of the labeled cells was put aside, and the cells were spun out of the media. 100 μL of the media was carefully added to wells in triplicate to avoid disturbing the pelleted cells. 100 μL of BATDA labeled cells were added to each well of the 96-well plate. Wells were saved for spontaneous release from target cells, and wells were prepared for maximum lysis of target cells by addition of 1% Triton-X. Monoclonal antibody (i.e., 5T4-mAb) or TriNKET® against 5T4 (i.e., 5T4-TriNKET®) were diluted in culture media, and 50 μL of diluted mAb or TriNKET® was added to each well. Rested NK cells were harvested from culture, cells were washed, and were resuspended at 10⁵-2.0×10⁶ cells/mL in culture media depending on the desired effector-to-target (E:T) ratio. 50 μL of NK cell suspension was added to each well of the plate to make a total of 200 μL culture volume. The plate was incubated at 37° C. with 5% CO₂ for 2-3 hours before developing the assay.

After culturing for 2-3 hours, the plate was removed from the incubator and the cells were pelleted by centrifugation at 200 g for 5 minutes. 20 μL of culture supernatant was transferred to a clean microplate provided by the manufacturer, and 200 μL of room temperature europium solution was added to each well. The plate was protected from the light and incubated on a plate shaker at 250 rpm for 15 minutes, then read using either Victor 3 or SpectraMax i3X instruments. % Specific lysis was calculated as follows:

% Specific lysis=((Experimental release−Spontaneous release)/(Maximum release−Spontaneous release))*100%

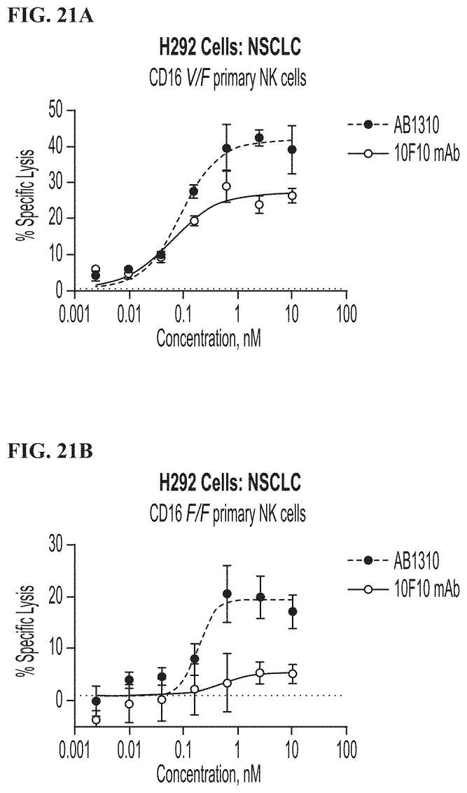

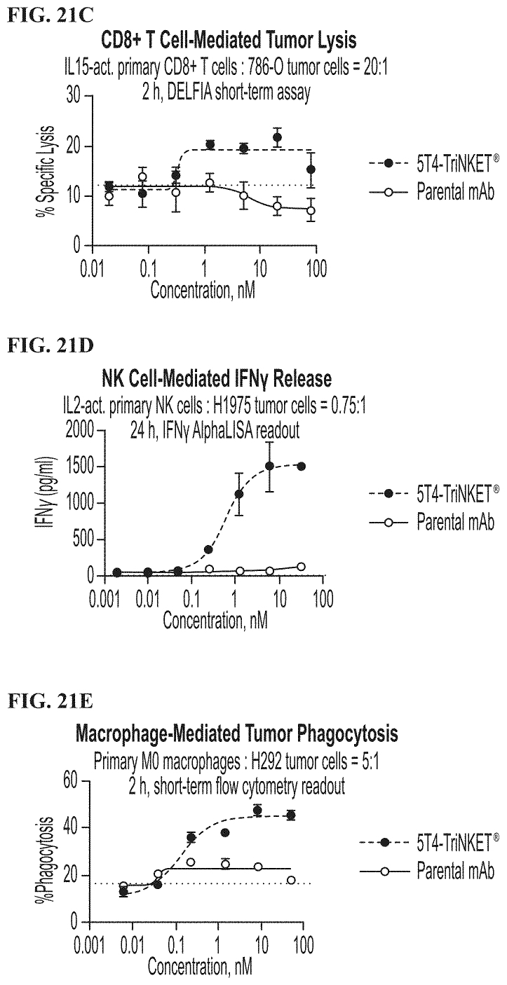

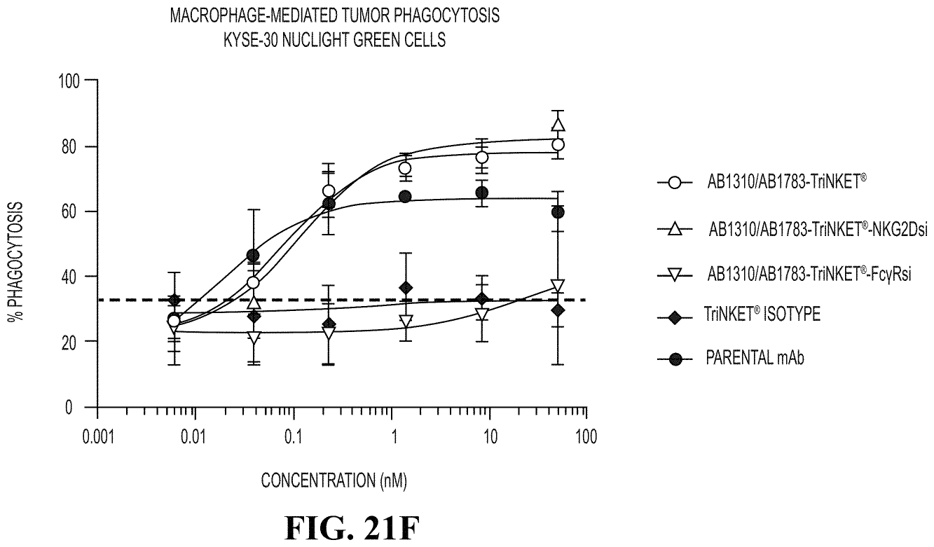

FIG. 21A and FIG. 21B show human NK cell-mediated lysis of H292 cells, in the presence of 5T4-TriNKET® or 5T4-mAb, using NK cells from 2 distinct healthy human donors (both V/F and F/F CD16-expressing NK cells). The 5T4-mAb demonstrated meaningful or negligible activity in an NK cell donor-dependent fashion, while 5T4-TriNKET® triggered superior maximal lysis compared to the mAb with sub-nM EC50 values in both contexts. In similar fashion, FIG. 22C and FIG. 22D show superior enhancement of NK cell-mediated lysis of primary 5T4-expressing human fibroblasts by 5T4-TriNKET® compared to 5T4-mAb (both V/F and F/F CD16-expressing NK cells). FIG. 21C shows CD8+ T cell-mediated lysis of 786-0 cells in the presence of 5T4-TriNKET® or 5T4 mAb. While inclusion of 5T4-TriNKET® triggered enhanced lysis of tumor cells by IL-15 pre-stimulated CD8+ T cells, 5T4-mAb activity above baseline was absent. FIG. 21D shows release of interferon-gamma (IFNγ) by 5T4+H1975 tumor cells induced by primary NK cells in the presence of 5T4-TriNKET® or 5T4-mAb. The IFNγ release induced by 5T4-mAb was minimal at all tested concentrations, whereas 5T4-TriNKET® induced superior IFNγ release, especially at higher concentrations. FIG. 21E shows phagocytosis of 5T4-expressing H292 cells by primary MO macrophages in in the presence of 5T4-TriNKET® or 5T4-mAb. FIG. 21F shows phagocytosis of 5T4-expressing KYSE-30 esophageal squamous cell carcinoma (EsoSCC) cells by primary M0 macrophages in the presence of 5T4-TriNKET® or 5T4-mAb. Against both tumor cell types, 5T4-mAb showed an induction of phagocytosis only slightly over baseline, whereas the 5T4-TriNKET® induced a large increase in phagocytosis. The data are summarized in Table 17.

TABLE 17
Summary of ADCP Activity of AB1310/AB1783-TriNKET ®

| | | AB1310/AB1783-TriNKET ® | | | Parental mAb | |
|---|---|---|---|---|---|---|
| NucLight ™ | Average | | | Max | | Max |
| Green Cell | 5T4 per | EC₅₀ | EC₉₀ | Phagocytosis | EC₅₀ | Phagocytosis |
| Line | Cell | (nM) | (nM) | (%) | (nM) | (%) |
| KYSE-30 | 47,000 | 0.089 ± 0.040 | 0.80 ± 0.36 | 57 ± 4 | 0.020 ± 0.008 | 48 ± 13 |
| NCI-H292 | 7,900 | 0.15 ± 0.02 | 1.3 ± 0.2 | 23 ± 6 | NR | 12 ± 1 |

ADCP: antibody-dependent cellular phagocytosis; EC50: concentration resulting in half-maximal response (based on 3-parameter, nonlinear regression curve fit); EC90: concentration resulting in 90% of maximal response (based on 3-parameter, nonlinear regression curve fit); Max: maximum increase mediated by test articles (based on 3-parameter, nonlinear regression curve fit); NR: not recorded. Mean ± standard deviation (SD) values were calculated using n = 3 healthy human macrophage donors. Each sample was run in duplicate.

A flow cytometry based PSR assay allows the filtering out of antibodies that have a higher probability to bind non-specifically to unrelated proteins. PSR assay correlates well with cross-interaction chromatography, a surrogate for antibody solubility, as well as with baculovirus particle enzyme-linked immunosorbent assay, a surrogate for in vivo clearance (Xu et. al (2013). Addressing polyspecificity of antibodies selected from an in vitro yeast presentation system: a FACS-based, high-throughput selection and analytical tool. Protein engineering design and selection, 26, 663-670).

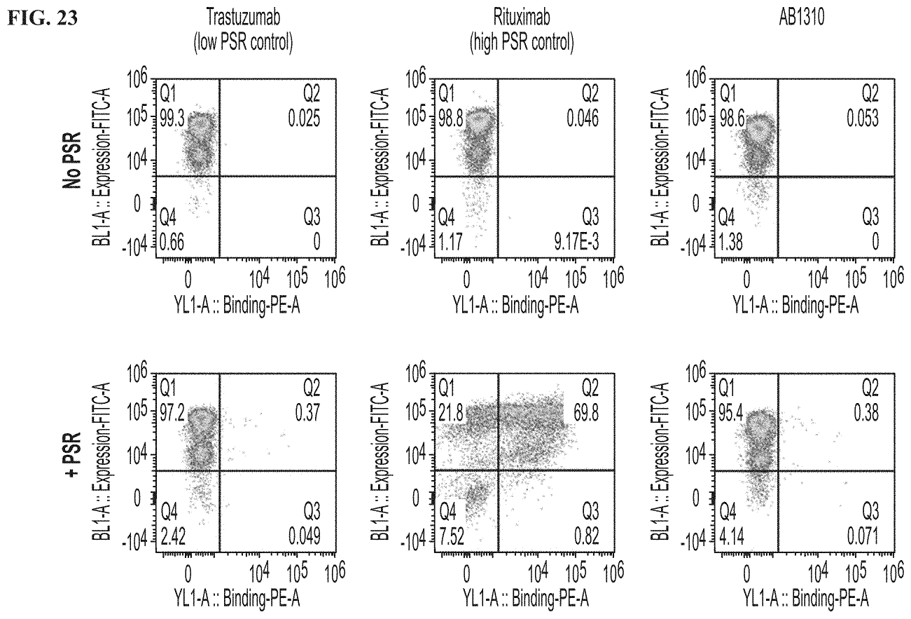

50 μL of 100 nM TriNKET® or control mAb in PBSF were incubated with pre-washed 5 μL protein A dyna beads slurry (Invitrogen, catalog #10001D) for 30 minutes at room temperature. TriNKET® or mAb bound magnetic beads were allowed to stand on a magnetic rack for 60 seconds and the supernatant was discarded. The bound beads were washed with 100 μL PBSF. Beads were incubated for 20 minutes on ice with 50 μL of biotinylated PSR reagent which was diluted 25-fold from the stock (Xu et. al., (2013) Protein engineering design and selection, 26, 663-670). Samples were put on the magnetic rack, supernatant discarded, and washed with 100 μL of PBSF. A secondary FACS reagent, to detect binding of biotinylated PSR reagent to TriNKETs® or control mAbs, was made as follows: 1:250 μL of Streptavidin-PE (Biologend, catalog #405204) and 1:100 donkey anti-human Fc were combined in PBSF. To each sample, 100 μL of the secondary reagents were added and allowed to incubate for 20 minutes on ice. The beads were washed twice with 100 μL PBSF, and samples were analyzed on a FACS Celesta (BD). AB1310/AB1783-TriNKET® was further shown to lack non-specific interactions by polyspecificity reagent (PSR) assay (FIG. 23).

### Example 2—Assessment of AB1310/AB1783-TriNKET® Functional and Physical Properties

### Manufacturability

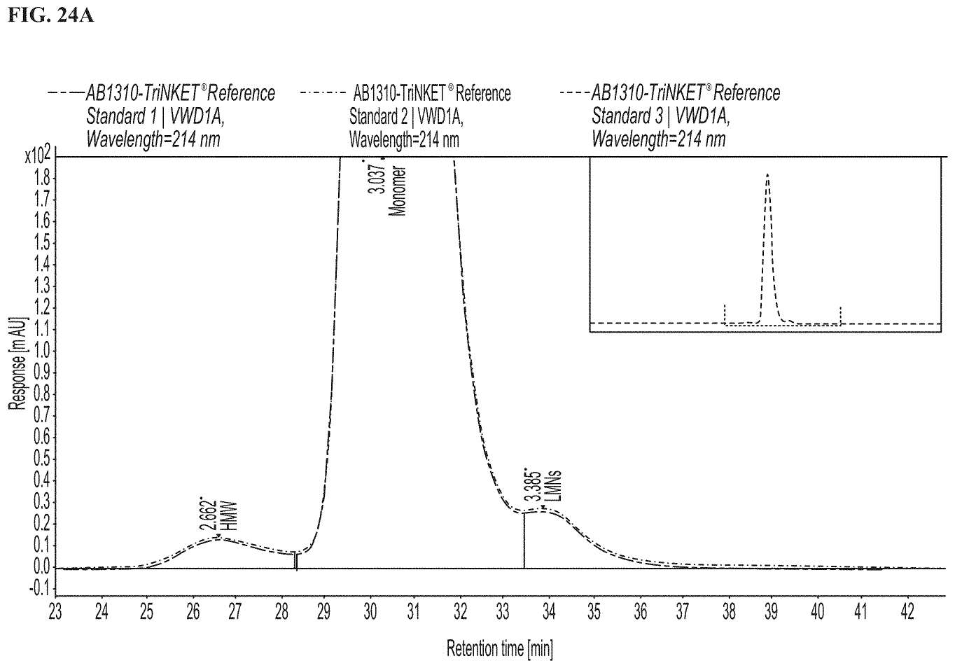

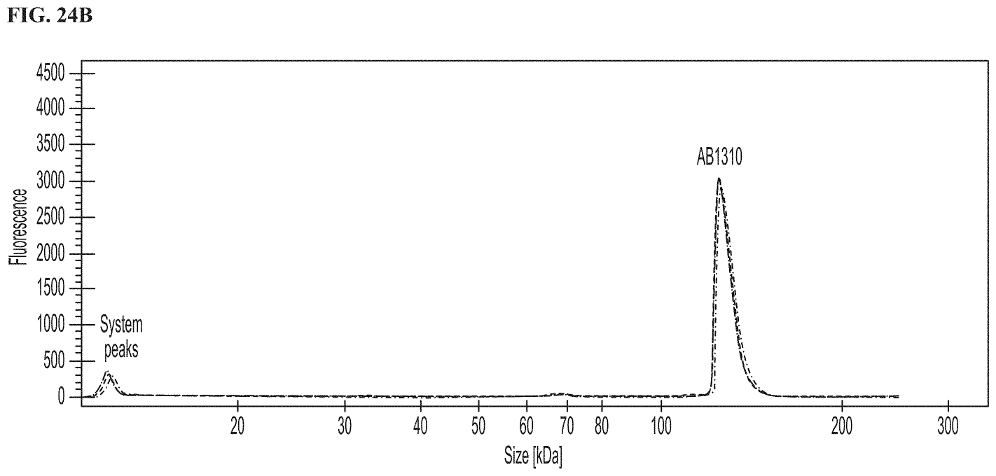

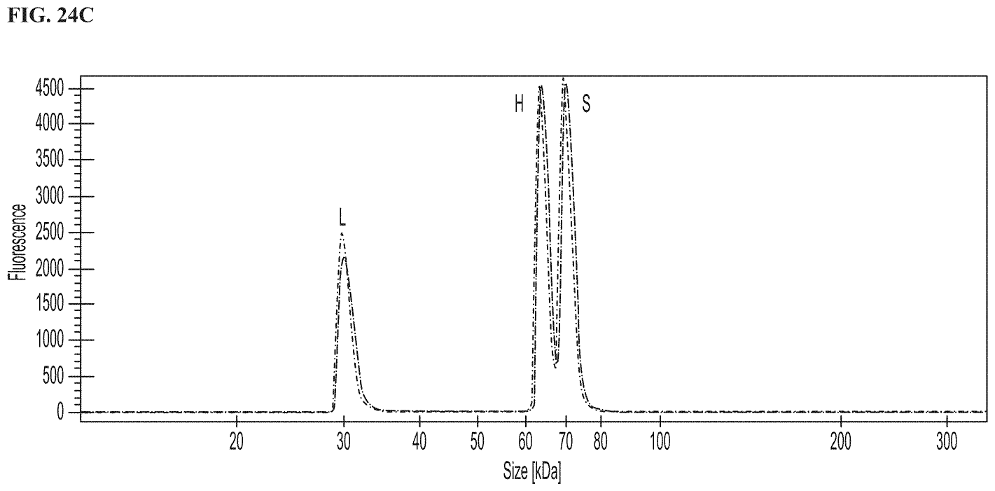

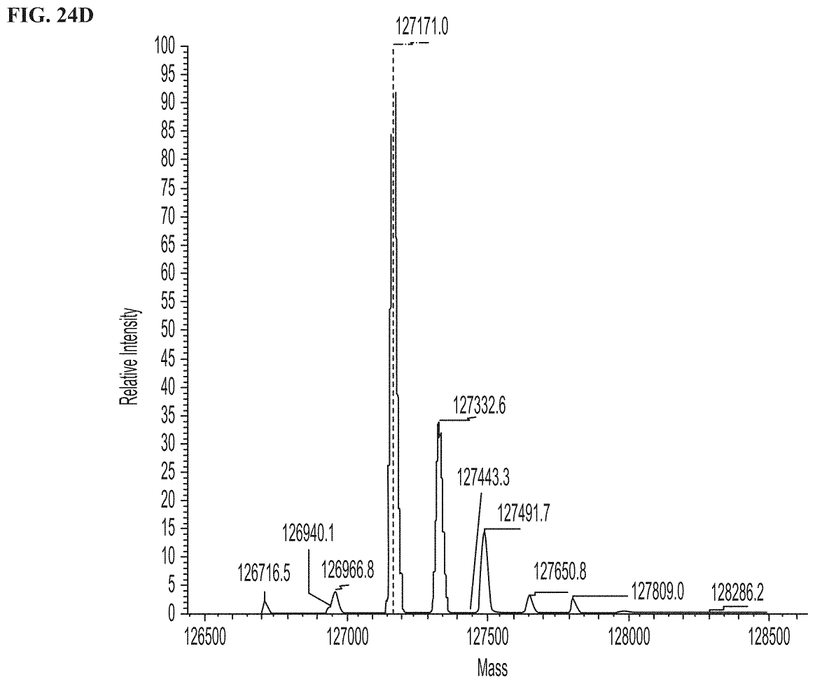

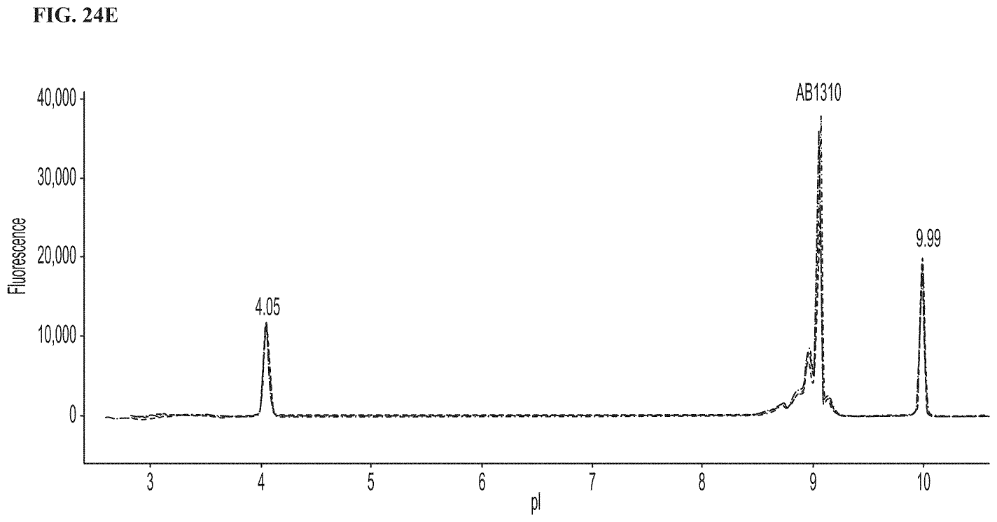

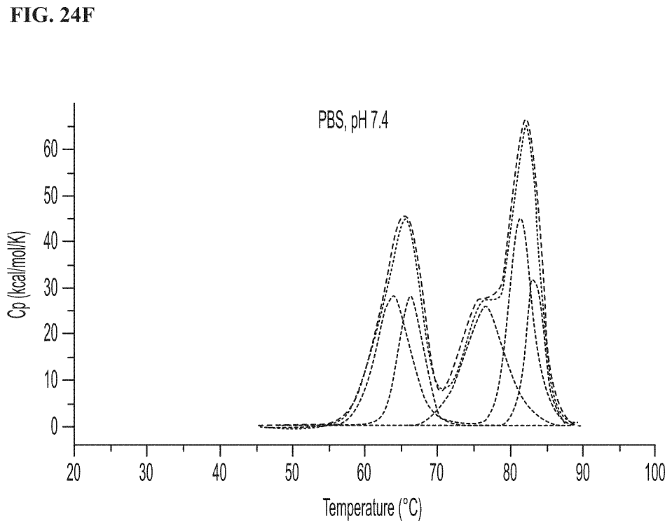

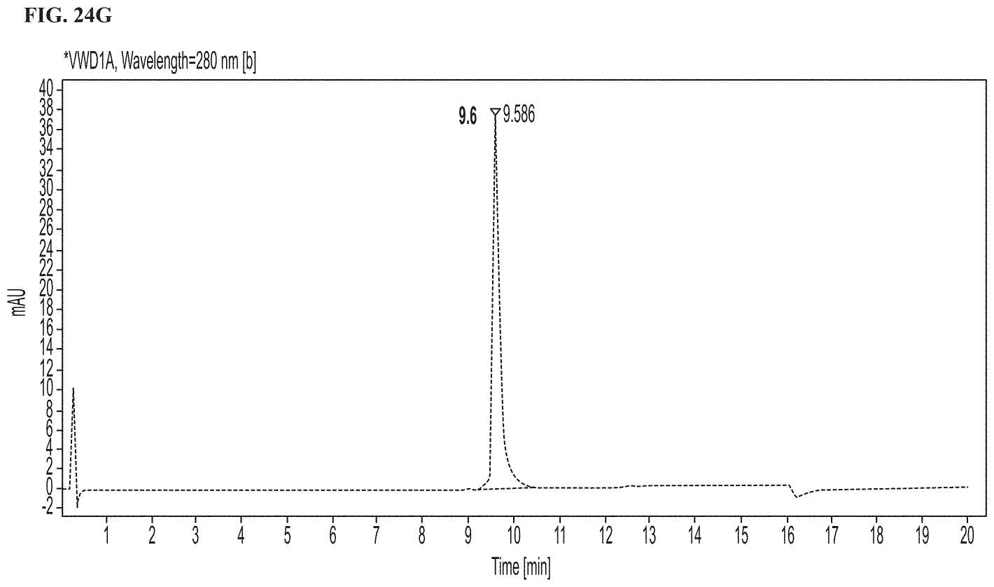

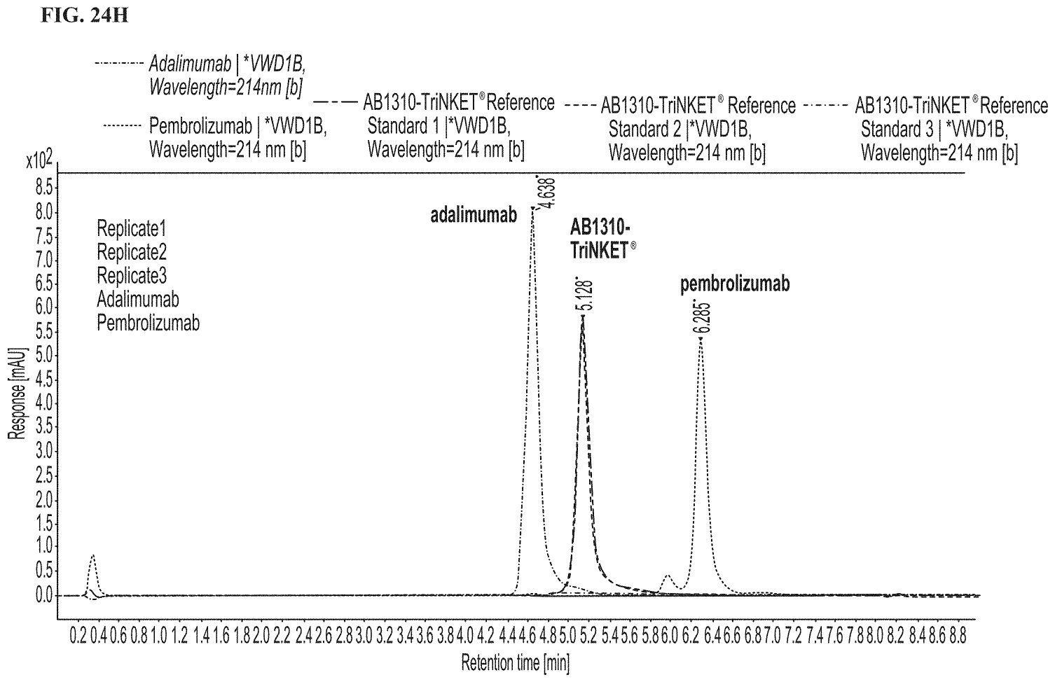

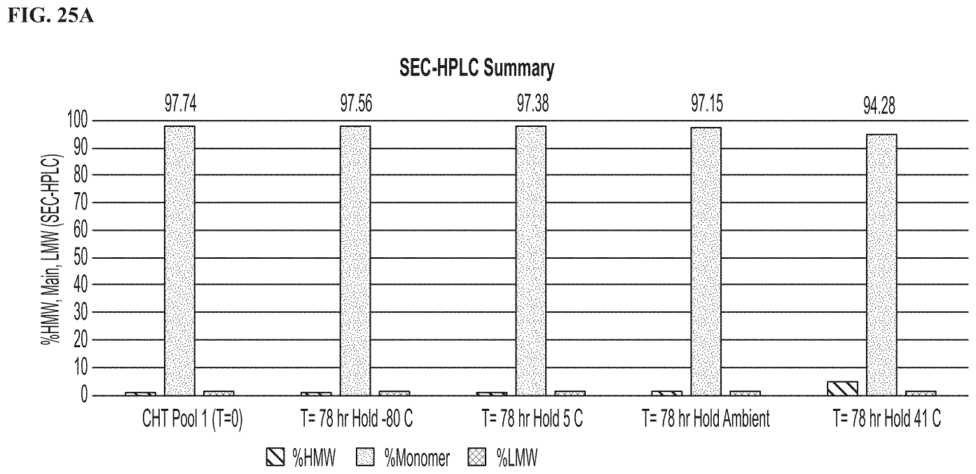

Manufacturability analysis showed that AB1310/AB1783-TriNKET® has favorable properties. The purity of AB1310/AB1783-TriNKET® was determined by size exclusion chromatography (SEC) (FIG. 24A) to monitor the formation of high molecular weight species (HMWS) and low molecular weight species (LMWS) as a function of storage conditions. Briefly, 5 μg of test material was injected onto an Agilent 1260 Infinity II high pressure liquid chromatography (HPLC) instrument with 1260 Quat Pump, 1260 Vialsampler, 1260 VWD. The sample was separated on a Waters Acquity BEH 200A SEC, 4.6 mm I.D.×15 cm, 1.7 μm column. SEC running buffer was PBS, pH 7.0, flowing at 0.40 ml/min. Absorbance was monitored at 214 nm, peak areas were manually integrated, and the percent of high molecular weight species (HMWS), low molecular weight species (LMWS), and monomer were reported.

Results were confirmed by non-reducing capillary electrophoresis (NR-CE) (FIG. 24B) and reducing capillary electrophoresis (R-CE) (FIG. 24C). The observed mass of purified AB1310/AB1783-TriNKET® was found to closely match the expected mass as measured by mass spectrometry (FIG. 24D). Results of mass spectrometry analysis of AB1310/AB1783-TriNKET® are summarized in Table 18.

TABLE 18
Mass Spectrometry Analysis of AB1310/AB1783
Intact Mass Results

| | |
|---|---|
| Observed Mass (Da) | 127,171.0 |
| Theoretical Mass (Da) | 127,170.1 |
| Mass Accuracy (ppm) | 7.1 |
| Modifications | 2 G0F |

AB1310/AB1783-TriNKET® was further characterized by capillary isoelectric focusing (cIEF) to assess its charge profile (FIG. 24E), differential scanning calorimetry (DSC) to assess its melting temperature (FIG. 24F), hydrophobic interaction chromatography (HIC) to assess its hydrophobicity (FIG. 24G). Hydrophobicity of AB1310/AB1783-TriNKET® as measured by HIC compared favorably to benchmark monoclonal antibodies adalimumab and pembrolizumab (FIG. 2411). Comparative HIC results are shown in Table 19.

For cIEF, briefly, samples were diluted to 1 mg/mL with MilliQ water, 15 μL of sample was added to 60 μL of master mix (water, methyl cellulose, Pharmalyte 3-10, arginine, pI markers 4.05 and 9.99), vortexed, and centrifuged briefly. 60 μL of sample was aspirated from the top of the solution and added to a 96-well plate and centrifuged before testing. The sample was separated for one minute at 1500 volts followed by 8 minutes at 3000 volts on a Maurice instrument (ProteinSimple, San Jose, Calif.).

For DSC, briefly, samples were prepared at 0.5 mg/mL in 1×PBS (Gibco, #10010-031) or alternative formulations. 325 μL was added to a 96-well deep well plate along with a matching buffer blank. Thermograms were generated using a MicroCal PEAQ DSC (Malvern, Pa.). Temperature was ramped from 20-100° C. at 60° C./hour. Raw thermograms were background subtracted, the baseline model was spline, and data were fitted using a non-two state model.

To perform HIC, briefly, injections of TriNKETs® (5 μg of protein) were injected onto an Agilent 1260 Infinity II high pressure liquid chromatography (HPLC) instrument equipped with a Sepax Proteomix HIC Butyl-NP5 5 um column at a flow rate of 1 mL/min and temperature of 25° C. Sample was eluted over a linear gradient from high salt buffer (100 mM sodium phosphate, 1.8 M ammonium sulfate, pH 6.5) to low salt buffer (100 mM sodium phosphate, pH 6.5) over the course of six minutes. Absorbance was monitored at both 214 and 280 nm, peaks were manually integrated, and retention times were reported

TABLE 19
Physical Properties of AB1310/AB1783-TriNKET ® and
other biologics

| Analyte | RT (min) |
|---|---|
| AB1310-TriNKET ® | 5.1 ± 0.00 |
| adalimumab | 4.6 |
| pembrolizumab | 6.3 |

### Stability Analysis of AB1310/AB1783-TriNKET®

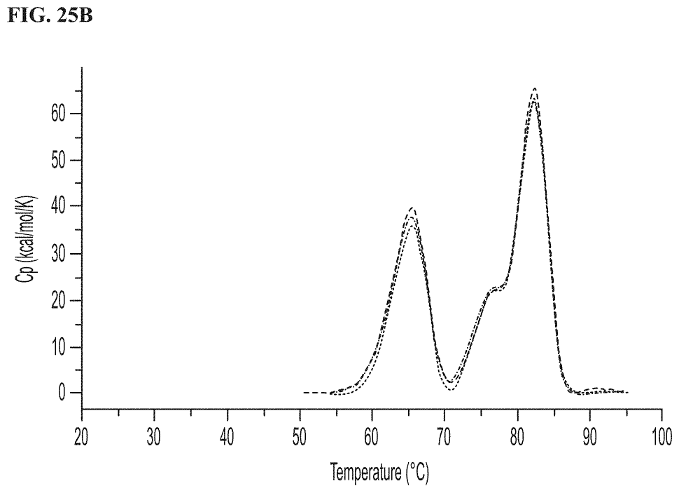

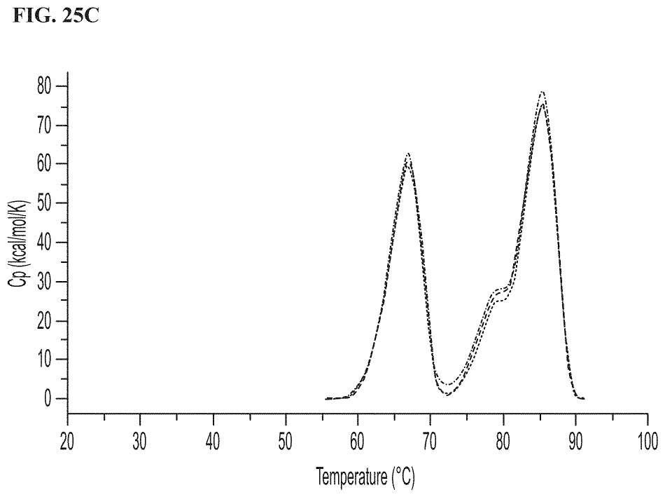

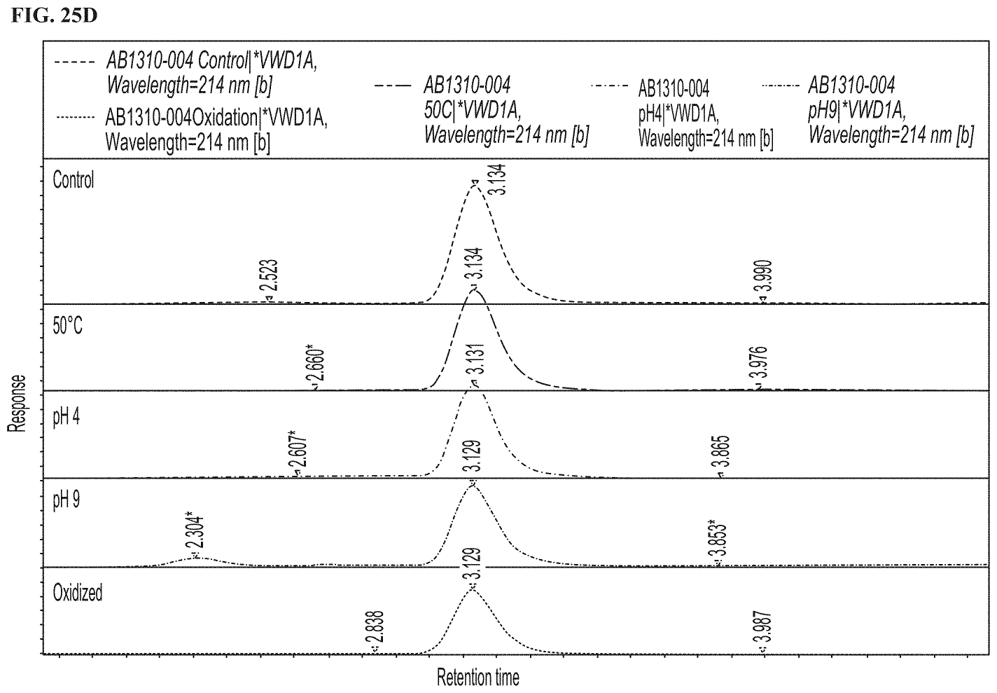

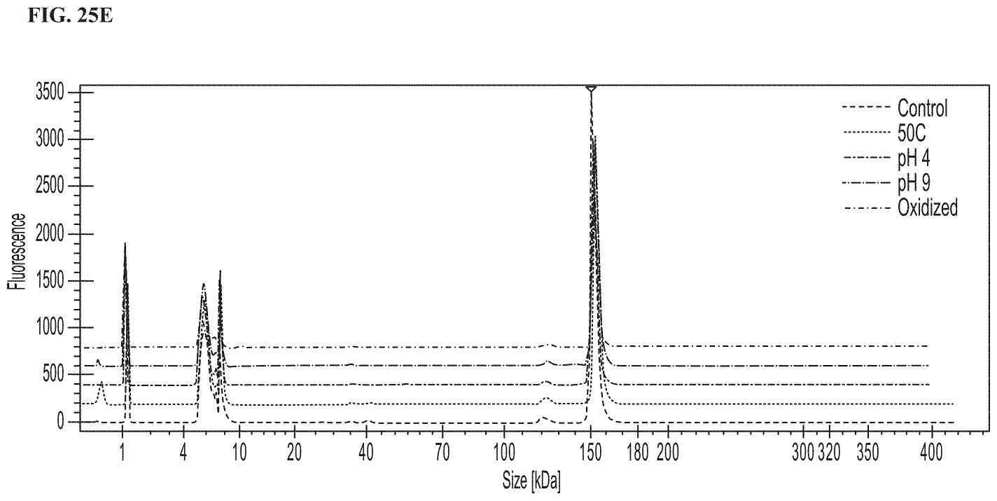

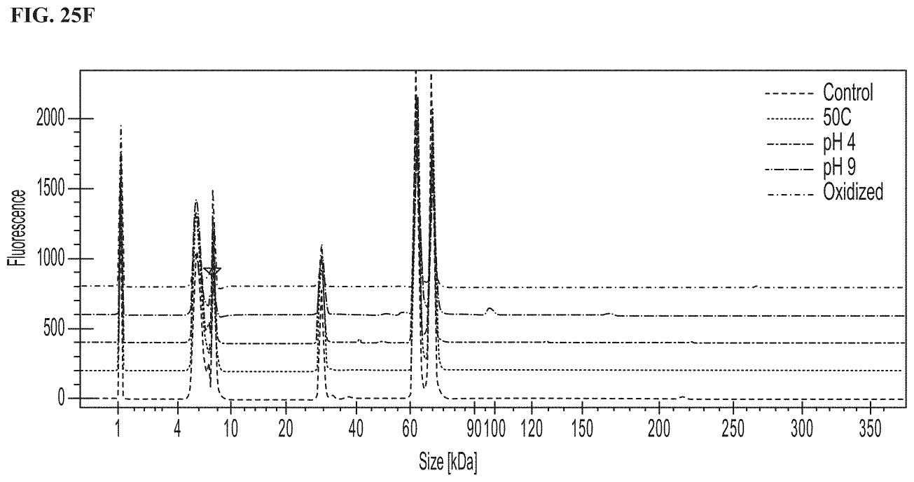

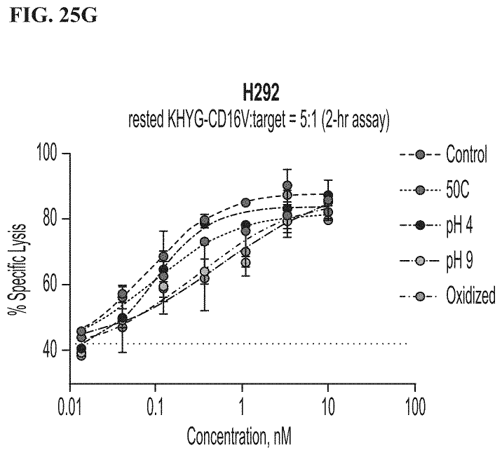

The stability of therapeutic proteins under various stresses (e.g. high temperature, low pH, high pH, and oxidizing conditions) is important to their development. The stability of AB1310-TriNKET® under various temperature conditions over 78 hours was assayed via SEC. Monomer purity of AB1310/AB1783-TriNKET® was not drastically altered after incubations at −80° C., 5° C., ambient temperature, or 41° C., indicating that AB1310/AB1783-TriNKET® retained proper folding (FIG. 25A). DSC analysis further showed that AB1310/AB1783-TriNKET® has high thermal stability in preformulation buffers PBS, pH 7.4 (FIG. 25B) and HST, pH 6.0 (FIG. 25C). DSC analysis of AB1310/AB1783-TriNKET® is summarized in Table 20.

TABLE 20
DSC Analysis of AB1310/AB1783-TriNKET ®
Thermal Stability in Preformulation Buffers

| | Tₒₙₛₑₜ | Tm₁ | Tm₂ | Tm₃ | Tm₄ |
|---|---|---|---|---|---|
| Buffer | (° C.) | (° C.) | (° C.) | (° C.) | (° C.) |
| PBS, pH 7.4 | 57.5 ± 0.5 | 63.7 ± 0.1 | 66.2 ± 0.1 | 76.8 ± 0.1 | 82.3 ± 0.0 |
| HST, pH 6.0 | 59.6 ± 0.2 | 66.2 ± 0.1 | 67.8 ± 0.1 | 78.9 ± 0.0 | 84.2 ± 0.0 |

SEC analysis also showed that AB1310/AB1783-TriNKET® retained proper folding after incubations at 50° C., under acidic conditions (pH 4), alkaline conditions (pH 9), and oxidizing conditions (FIG. 25D). NR-CE and R-CE analysis further showed that AB1310/AB1783-TriNKET® did not undergo degradation under these conditions (FIGS. 25E and 25F). Finally, AB1310/AB1783-TriNKET® maintained its ability to bind 5T4 and induce NK-cell mediated lysis of 5T4-expressing cells (FIG. 25G). Results of analyses of AB1310/AB1783-TriNKET® properties under stress conditions are summarized in Table 21.

TABLE 21
Analysis of AB1310/AB1783-TriNKET ® physical properties under stress conditions

| | | | | | | SEC | | | NR CE-SDS |
|---|---|---|---|---|---|---|---|---|---|
| | | | | | Temp | HMWS | Main | LMWS | LMWS |
| Analyte | Stress | Buffer | Time | | (° C.) | (%) | (%) | (%) | (%) |
| AB1310/ | Control | HST, pH 6 | 7 | days | 4° C. | 3.3 | 96.1 | 0.6 | 2.2 |
| AB1783- | pH | Tris, pH 9 | 7 | days | 40° C. | 13.0 | 86.6 | 0.4 | 4.6 |
| TriNKET ® | | Acetate pH 4 | 7 | days | 40° C. | 5.1 | 94.4 | 0.5 | 1.9 |
| | Temp. | HST, pH 6 | 7 | days | 50° C. | 2.2 | 97.1 | 0.7 | 3.1 |
| | Oxidation | PBS, 0.02% H₂O₂ | 24 | hrs | RT | 2.7 | 96.9 | 0.3 | 2.3 |

| | | | R CE- | | | | | |
|---|---|---|---|---|---|---|---|---|
| | NR CE-SDS | | SDS | h5T4 binding | | | Cytolysis | |
| | Main | HMWS | Purity | kₐ | k_{d} | k_{D} | Rel. | Rel. |
| Analyte | (%) | (%) | (%) | (1/Ms) | (1/s) | (nM) | potency | max |
| AB1310/ | 97.8 | N/A | 99.4 | 1.2E+05 | 9.1E−04 | 7.8 | 1.00 | 1.0 |
| AB1783- | 95.4 | N/A | 95.6 | 1.1E+05 | 1.6E−03 | 15.1* | 0.18** | 1.0 |
| TriNKET ® | 98.1 | N/A | 99.0 | 1.2E+05 | 9.1E−04 | 7.6 | 0.81 | 0.9 |
| | 96.9 | N/A | 98.2 | 1.1E+05 | 8.8E−04 | 7.6 | 0.87 | 0.9 |
| | 97.7 | N/A | 100.0 | 1.2E+05 | 8.6E−04 | 7.0 | 0.41** | 1.0 |

*heterogeneous binding, approximate value
**mean of 2 independent assay runs

Assessment of Surface Binding to h5T4 In Vitro

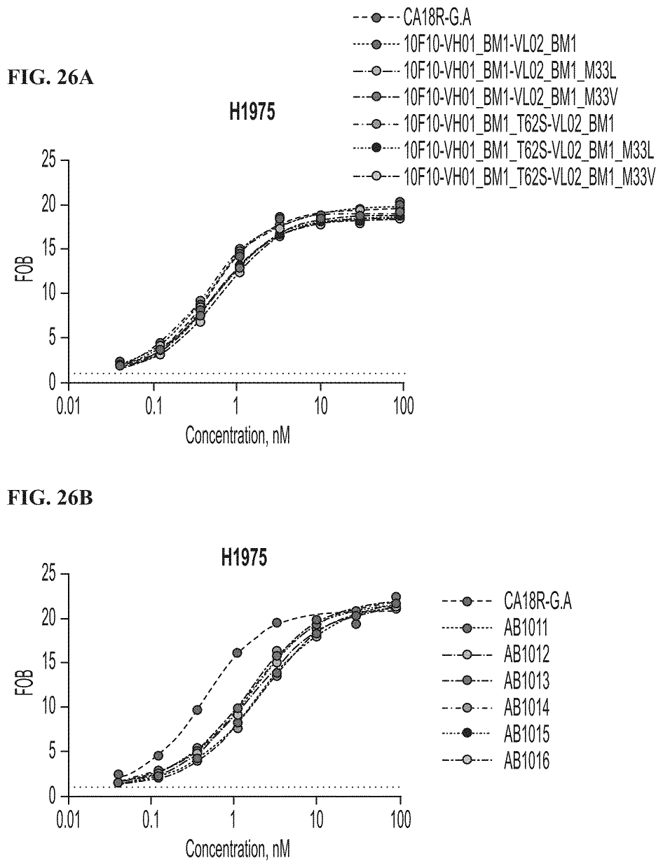

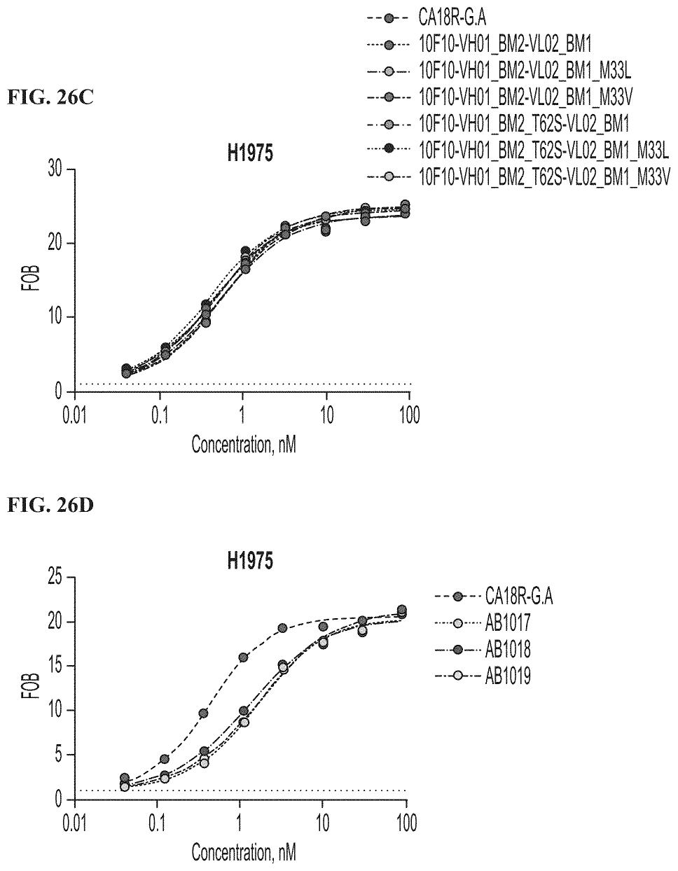

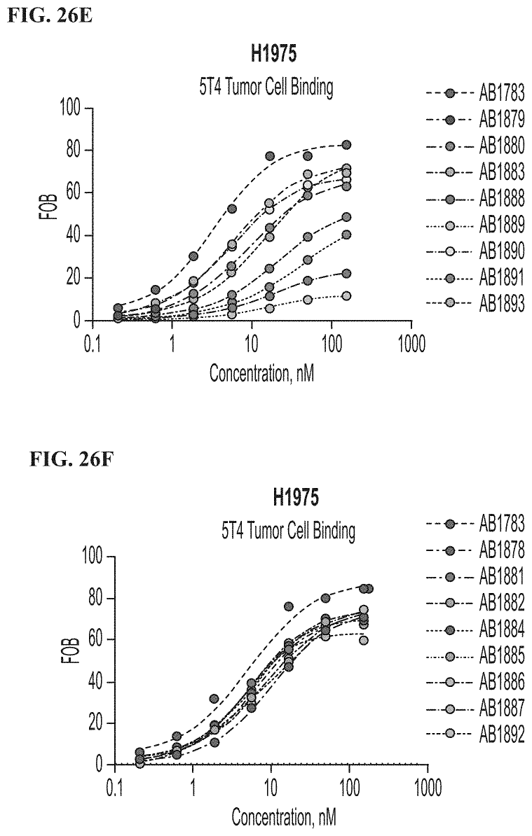

Identified 5T4-binding clones 10F10 and 11F09 were both shown to maintain binding to 5T4 tumor cells H1975 following humanization and sequence liability alteration (FIG. 26A-26D) using the binding affinity assay described above. FIG. 26E and FIG. 26F are graphs showing binding (fold over background (FOB)) of various concentrations of humanized 5T4 binders.

AB1310/AB1783-TriNKET® bound with single-digit nanomolar relative affinity (1.5-7.6 nM EC50 values) on a panel of tumor cell lines representing a range of 5T4 expression, and did not bind a 5T4 knockout line, demonstrating high affinity and specificity, shown in Table 22.

TABLE 22
Binding of AB1310/AB1783-TriNKET ®
to 5T4-expressing tumor cell lines

| Target | | 5T4 | Cell binding |
|---|---|---|---|
| Cell line | Origin | per cell | EC50 (nM) |
| KYSE-30 | HNSCC | 61,000 | 2.5 |
| HCC4006 | NSCLC-adeno | 53,000 | 7.6 |
| FaDu | HNSCC | 22,000 | 7.1 |
| H1975 | NSCLC - adeno | 22,000 | 1.9 |
| Detroit 562 | HNSCC | 21,000 | 2.7 |
| BT474 | BRC | 20,000 | 3.7 |
| 786-O | RCC | 18,000 | 2.7 |
| H1703 | NSCLC - SCC | 15,000 | 2.6 |
| HCT116 | CRC | 14,000 | 2.1 |
| H292 | NSCLC | 12,000 | 1.5 |
| HT29 | CRC | 5,000 | 4.1 |
| H2172 | NSCLC | 0 | N/A |
| H292-5T4KO | NSCLC | 0 | N/A |

### AB1310/AB1783-TriNKET® Mediated Cell Lysis

DELFIA cytotoxicity assays were performed to assess AB1310/AB1783-mediated cell lysis of tumor cell lines. Briefly, 5T4+ target cells were pelleted, and washed with 1×HBS. Target cells were resuspended in pre-warmed RPMI primary cell culture media at 106 cells/mL. BATDA reagent (bis(acetoxymethyl) 2,2′:6′,2″-terpyridine-6,6″-dicarboxylate) was diluted 1:400 into the cell suspension. Cells were mixed and incubated at 37° C. with 5% CO2 for 15 minutes. The labeled target cells were washed 3× with 1×HBS and resuspended at 5×104 cells/mL in RPMI primary cell culture media.

Rested human NK cells were removed from culture and pelleted, the cells were resuspended in RPMI primary cell culture media at 0.5×10⁶ cells/mL. 4× test articles were prepared in RPMI primary cell culture media. In a round bottom TC 96-well plate, 100 μl of labeled target cells, 50 μl of 4× TriNKET®/mAb, and 50 μl of effector cells were added. Control wells for background were prepared by pelleting labeled target cells, and 100 μl of the supernatant was added to background wells, containing 100 μl of RPMI primary cell culture media. Spontaneous release wells were prepared by adding 100 μl of labeled target cells to wells containing 100 μl of RPMI primary cell culture media. Maximum release wells were prepared by adding 100 μl of labeled target cells to wells containing 80 μl of RPMI primary cell culture media and 20 μl of 10% TritonX-100 solution. The assay plate was incubated at 37° C. with 5% CO2 for 2-3 hours.

The assay plate was removed from the incubator and the plate was centrifuged to pellet cells. 20 μl of supernatant was removed from each well and transferred to a clean 96-well yellow DELFIA assay plate. 200 μl of room temperature Europium solution was added to each well and the plate was placed on a plate shaker 15 minutes at 250 RPM.

AB1310/AB1783-TriNKET® enhanced NK cell-mediated lysis of tumor cell lines representing a range of indications and 5T4 expression levels, as shown in Table 23. AB1310/AB1783-TriNKET® enhanced NK mediated lysis with subnanomolar EC50 values ranging from 0.072 to 0.42 nM in cell lines representing a range of 5T4 expression. Similarly, AB1310/AB1783 enhanced NK-mediated lysis of primary CAFs, as shown in Table 24.

TABLE 23
NK cell-mediated lysis by AB1310/AB1783-TriNKET ®

| | | | AB1310/AB1783- | | | |
|---|---|---|---|---|---|---|
| | | | TriNKET ® | | 10F10 mAb | |
| | | | EC50 | Max enh. | EC50 | Max enh. |
| Target cell | Origin | 5T4/cell | (nM) | lysis (%) | (nM) | lysis (%) |
| KYSE-30 | HNSCC | 61,000 | 0.072 | 15 | 0.085 | 12 |
| HCC4006 | NSCLC- | 53,000 | 0.24 | 13 | 0.25 | 12 |
| | adeno | | | | | |
| FaDu | HNSCC | 22,000 | 0.19 | 31 | 0.17 | 12 |
| H1975 | NSCLC - | 22,000 | 0.29 | 11 | 0.18 | 3.5 |
| | adeno | | | | | |
| Detroit 562 | HNSCC | 21,000 | 0.23 | 19 | 0.14 | 11 |
| BT474 | BRC | 20,000 | 0.14 | 19 | 0.20 | 12 |
| 786-O | RCC | 18,000 | 0.19 | 40 | 0.16 | 13 |
| H1703 | NSCLC - | 15,000 | 0.14 | 29 | 0.047 | 7.6 |
| | squam | | | | | |
| HCT116 | CRC | 14,000 | 0.097 | 23 | 0.039 | 7.2 |
| H292 | NSCLC | 12,000 | 0.10 | 44 | 0.17 | 24 |
| HT29 | CRC | 5,000 | 0.42 | 17 | ND | 0.2 |

TABLE 24
NK cell-mediated lysis of CAFs by AB1310/AB1783-TriNKET ®

| | EC50 | Max | EC50 | Max |
|---|---|---|---|---|
| Sample | (nM) | (%) | (nM) | (%) |
| AB1310-TriNKET ® | 0.081 | 36 | 0.19 | 10 |
| Parental mAb | 0.12 | 30 | — | 3.6 |

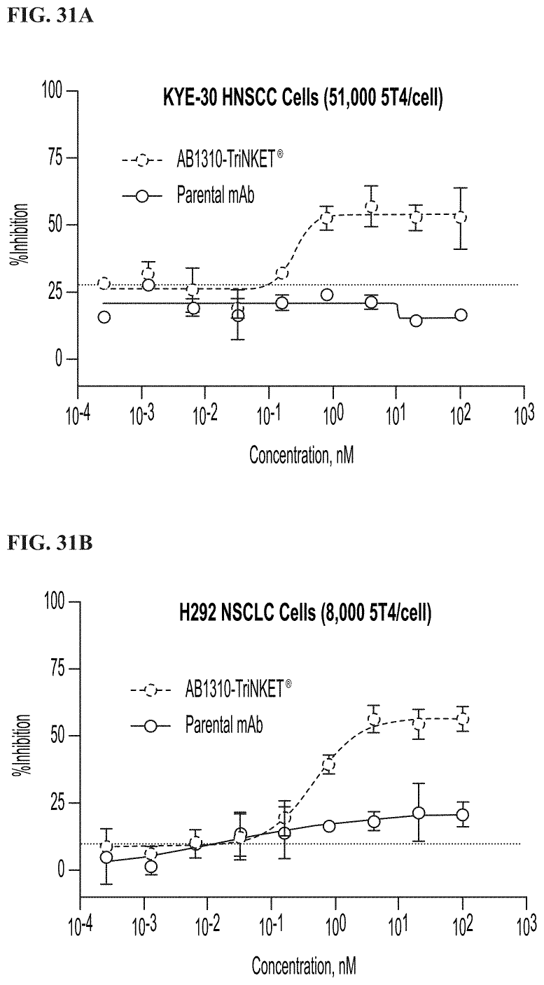

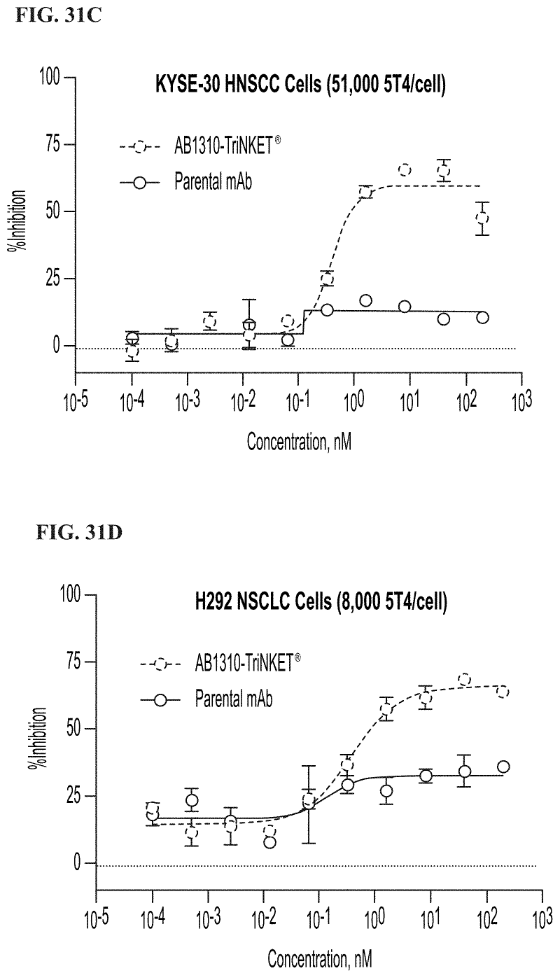

NK-cell mediated lysis enhanced by AB1310/AB1783-TriNKET® was sustained over a prolonged period. Two 5T4⁺ tumor cell lines (KYSE-30 and H292) expressing NucLight™ Green were incubated in 50% o human serum with rested primary NIK cells at a ratio of 5:1 NIK cells to tumor cells or with PBMCs at a ratio of 20:1 PBMCs to tumor cells. Cells were incubated in an IncuCyte® S3 for 72 hours. Percent inhibition was calculated as follows: % Inhibition=(1−((Green object count time X)/(green object count time zero)))*100%. AB1310/17830-TriNKET® enhanced killing of 5T4⁺ cells mediated by primary NK cells (FIG. 31A and FIG. 31B) or PBMCs (FIG. 31C and FIG. 31D) over the 72 hour incubation period.

Interaction of AB1310 AB1783-TriNKET® with NKG2D and CD16

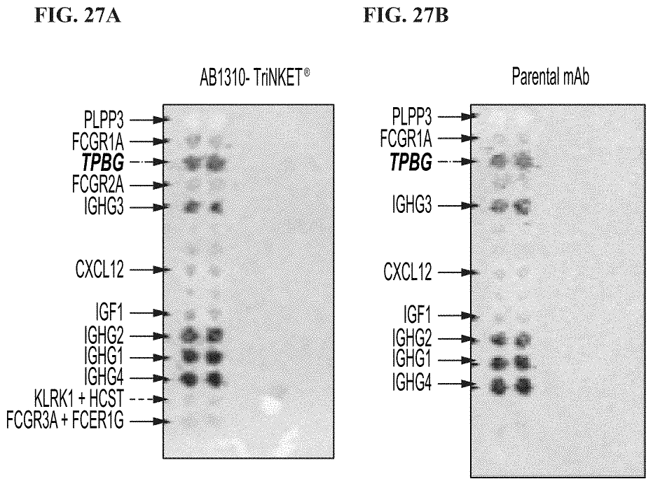

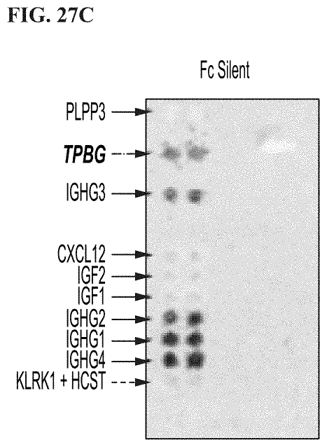

Binding of AB1310/AB1783-TriNKET® against plasma membrane proteins on HEK293 cell was assessed by protein array. As shown in FIG. 27A, AB1310/AB1783-TriNKET® specifically interacted with the primary target 5T4 (TPBG). Weak binding of AB1310/AB1783-TriNKET® to NKG2D-DAP10 was also observed (KLRK1+HCST). Specificity for 5T4 was maintained in the parental mAb (FIG. 27B), and binding to NKG2D was not observed. In an Fc-silent variant, in which silencing mutations were introduced to inhibit effector function of AB1310/AB1783-TriNKET®, binding to both 5T4 and NKG2D was maintained (FIG. 27C).

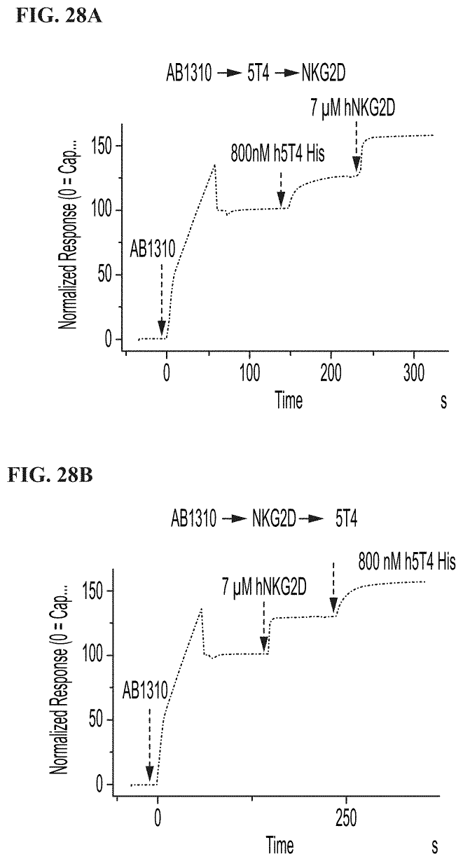

It was further demonstrated that AB1310/AB1783-TriNKET® achieved simultaneous co-engagement of 5T4 and NKG2D targeting arms, as shown in FIG. 28A (5T4 binding first, followed by NKG2D) and FIG. 28B (NKG2D binding first, followed by 5T4).

Briefly, AB1310/AB1783-TriNKET® was diluted in 1×HBS-EP+ buffer containing 0.1 mg/mL BSA and was captured on an anti-human Fc surface of CM5 chip at a flow rate of 5 μL/min for 60 sec to achieve capture level of 150-250 RU. The net difference between baseline signal and the signal after completion of AB1310-TriNKET® injection representing the amount of AB1310/AB1783-TriNKET® captured was recorded. h5T4-His (800 nM) or mFc-hNKG2D (7 μM) was injected over captured AB1310/AB1783 at 20 μL/min for 90 sec to reach saturation. This injection was immediately followed by an injection of pre-incubated mixture of h5T4-His (800 nM) and mFc-hNKG2D (7 μM) at a flow rate of 20 μL/min for 90 sec with the use of the A-B-A injection command in the Biacore 8K control software (second target was pre-mixed with the first target to assure all binding sites for the first target are occupied). The chip was regenerated by two 20 sec pulses of 10 mM Glycine (pH 1.7) at 100 μL/min. The experiment was conducted at 37° C. and 1×HBS-EP+ buffer containing 0.1 mg/mL BSA was used as a running buffer. Binding of each antigen, expressed in RU, was recorded as the net difference between the baseline signal prior and 12 seconds after the injection of individual antigen. An average relative binding ratio of each target bound to AB1310/AB1783-TriNKET® unoccupied with another target (injected first) was assigned a value of 1.0. An average relative binding stoichiometry of each target bound to captured AB1310/AB1783-TriNKET® that is already saturated with the other target (injected second) was expressed as a fraction of the full capacity binding to unoccupied AB1310/AB1783-TriNKET®. Stoichiometries are provided in Table 25.

Importantly, these results demonstrate that binding of 5T4 to AB1310 AB1783-TriNKET® does not impact binding of NKG2D, binding of NKG2D to AB1310 AB1783-TriNKET® does not impact binding of 5T4, and both binding arms can be engaged simultaneously

TABLE 25
5T4-NKG2D binding stoichiometries of
AB1310/AB1783-TriNKET ®

| | h5T4, | NKG2D, |
|---|---|---|
| | relative binding | relative binding |
| Experimental setup | stoichiometry | Stoichiometry |
| Target Bound to AB1310/AB1783- | 1.00 | 1.00 |
| TriNKET ® unoccupied with another | | |
| target (injected first) | | |
| Target Bound to AB1310/AB1783- | 1.04 ± 0.01 | 1.12 ± 0.03 |
| TriNKET ® saturated with another | | |
| target (injected second) | | |

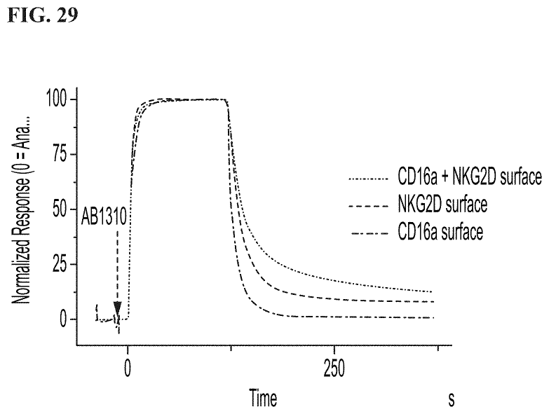

Synergistic binding of NKG2D and CD16a was assessed. Briefly, synergistic NKG2D and CD16a binding was evaluated by SPR as described above with modifications. hNKG2D alone, CD16a F158 allele alone and the mixture of hNKG2D and CD16a F158 were amino-coupled to the surface of the CM5 Series S Biacore chip. 1.5 μM AB1310/AB1783-TriNKET® was injected for 120 seconds at 20 μL/min. Dissociation phase was observed for 180 seconds when regeneration was not needed and 1200 seconds when natural regeneration of the surface (almost complete dissociation of analyte) was needed between the cycles at the same flow rate. 1×HBS-EP+ buffer was used as running and sample dilution buffer. Resulting sensorgrams were normalized to the “analyte binding late” report point and qualitatively assessed. Additionally, binding of AB1310/AB1783-TriNKET® displayed enhanced engagement by binding both NKG2D and CD16a, as demonstrated by the sensorgram in FIG. 29. This simultaneous engagement resulted in an avidity-improved off-rate.

### Activation of NK Cells by AB1310/AB1783-TriNKET®

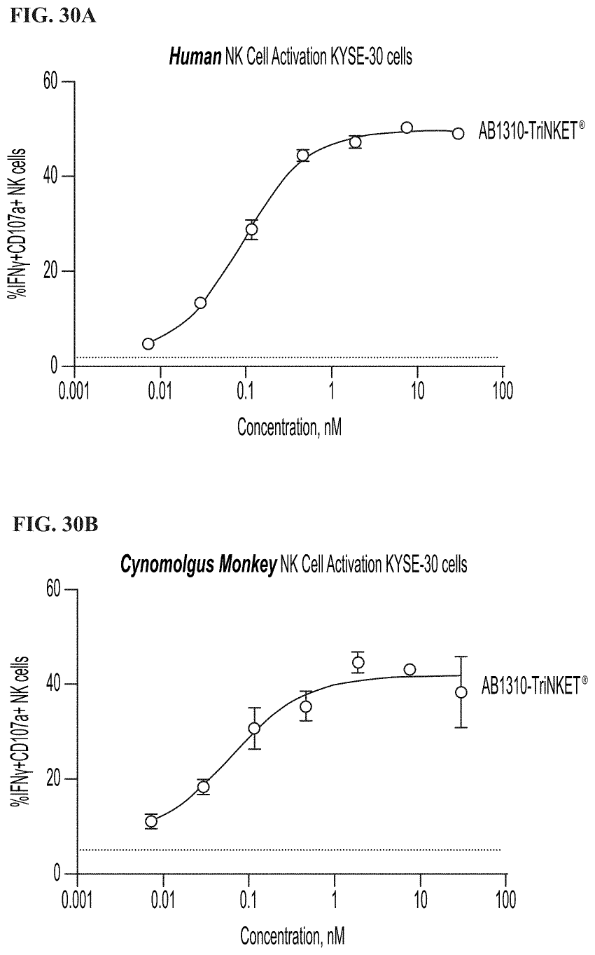

The ability of AB1310/AB1783-TriNKET® to activate human and cynomolgus monkey NK cells was assessed. Briefly, peripheral blood mononuclear cells (PBMCs) were isolated from human or cynomolgus peripheral blood buffy coats using density gradient centrifugation. NK cells (CD3⁻CD56⁺) were isolated using negative selection with magnetic beads from PBMCs, and the purity of the isolated NK cells was typically >95%. Isolated NK cells were then cultured in media containing 100 ng/mL IL-2 for 24-48 hours before they were transferred to the wells of a microplate to which the NKG2D-binding domains were adsorbed, and cultured in the media containing fluorophore-conjugated anti-CD107a antibody, brefeldin-A, and monensin. NK-cells were co-cultured with 5T4+-KYSE-30 cells. Following co-culture, NK cells were assayed by flow cytometry using fluorophore-conjugated antibodies against CD3, CD56 and IFN-gamma. CD107a and IFN-gamma staining were analyzed in CD3⁻CD56⁺ cells to assess NK cell activation. Similar levels of NK cell activation by were observed for both human (FIG. 30A) and cynomolgus (FIG. 30B) cells. Results are summarized in Table 26.

TABLE 26
NK Cell Activation by AB1310/AB1783-TriNKET ®

| | | | Human | | Cynomolgus monkey | |
|---|---|---|---|---|---|---|
| | | | EC50 | Max IFNγ+/ | EC50 | Max IFNγ+/ |
| Target cell | Origin | 5T4/cell | (nM) | CD107a+(%) | (nM) | CD107a+(%) |
| KYSE-30 | HNSCC | 61,000 | 0.13 ± 0.03 | 34 ± 11 | 0.06 ± 0.02 | 39 ± 9 |
| H292 | NSCLC | 12,000 | 0.35 ± 0.13 | 26 ± 12 | 0.06 ± 0.02 | 42 ± 12 |

### Binding Affinities of Various 5T4 TriNKETs®

Results from a binding assay comparing the binding affinities of different 5T4 TriNKETs® are provided below in Table 27 and Table 28.

TABLE 27

| | | Max Fold Over |
|---|---|---|
| TriNKET ® | EC50 (nM) | Background (FOB) |
| AB1783 | 3.424 | 82.8 |
| 10F10 F3′ LH H(T62S) CHO opt. | | |
| AB1879 | 9.183 | 62.9 |
| 11F09-IGHV3-48*01 BM1-YB | | |
| AB1880 | 21.74 | 48.5 |
| 11F09-IGHV3-21*05 BM2-YB | | |
| AB1883 | 6.019 | 69.4 |
| 11F09-IGHV3-11*01 BM2-YB | | |
| AB1888 | 18.51 | 22.1 |
| 11F09-IGHV3-21*05 BM1-YB | | |
| AB1889 | 20.68 | 11.7 |
| 11F09-IGHV3-48*01 BM2-YB | | |
| AB1890 | 5.616 | 65.9 |
| 11F09-IGHV3-23*03 BM1-YB | | |
| AB1891 | 50.54 | 40.1 |
| 11F09-IGHV3-11*01 BM1-YB | | |
| AB1893 | 15.86 | 70.8 |
| 11F09-IGHV3-23*03 BM2-YB | | |

TABLE 28

| | | Max Fold Over |
|---|---|---|
| TriNKET ® | EC50 (nM) | Background (FOB) |
| AB1783 | 5.33 | 85.0 |
| 10F10 F3′ LH H(T62S) CHO opt. | | |
| AB1878 | 10.09 | 71.6 |
| 10F10-IGHV3-21*05 BM1 | | |
| AB1881 | 7.278 | 72.5 |
| 10F10-IGHV3-23*03 BM2 | | |
| AB1882 | 8.551 | 71.8 |
| 10F10-IGHV3-48*01 BM1 | | |
| AB1884 | 6.075 | 70.5 |
| 10F10-IGHV3-48*01 BM2 | | |
| AB1885 | 5.365 | 68.0 |
| 10F10-IGHV3-11*01 BM2 | | |
| AB1886 | 5.652 | 73.5 |
| 10F10-IGHV3-11*01 BM1 | | |
| AB1887 | 10.21 | 75.0 |
| 10F10-IGHV3-21*05 BM2 | | |
| AB1892 | 4.207 | 60.0 |
| 10F10-IGHV3-23*03 BM1 | | |

### Example 3—Assessment of Formulations of AB1310/AB1783-TriNKET®

### pH Screening

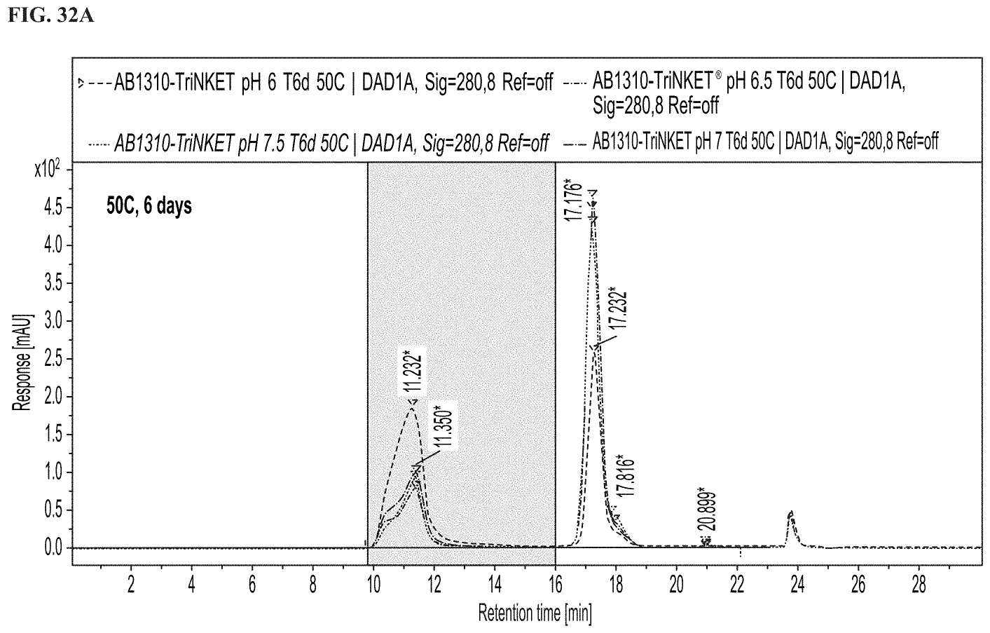

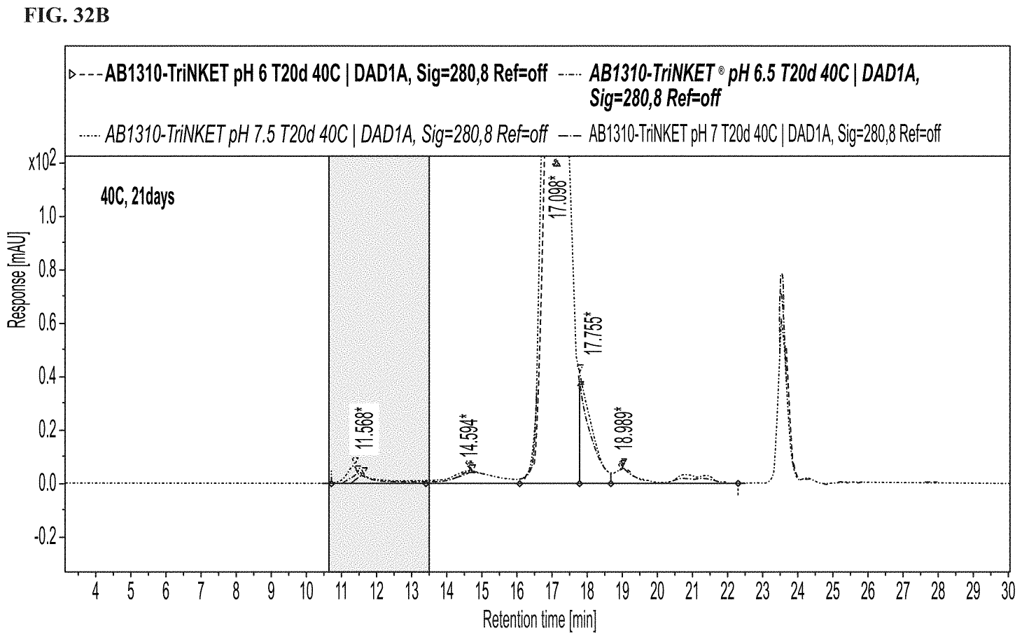

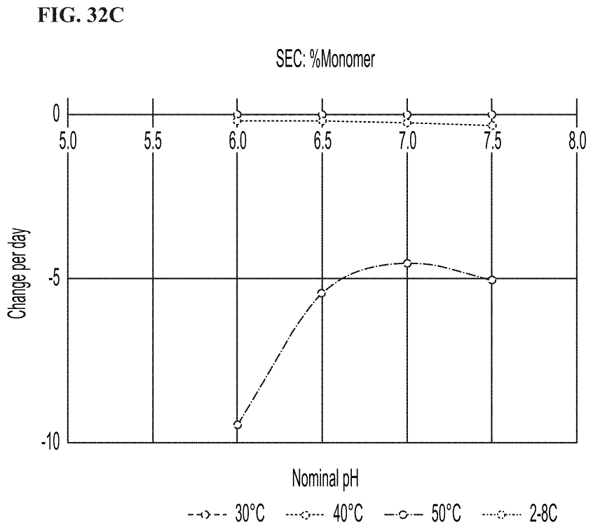

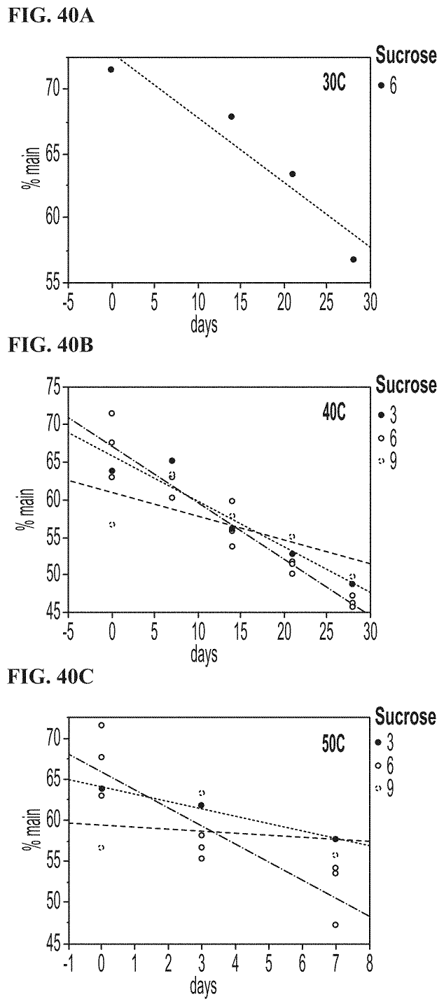

The purity of AB1310/AB1783-TriNKET® in formulations at various pH levels was determined by size exclusion chromatography (SEC) to monitor the formation of high molecular weight species (HMWS) and low molecular weight species (LMWS) over time as a function of storage conditions. Briefly, 5 μg of test material was injected onto an Agilent 1260 Infinity II high pressure liquid chromatography (HPLC) instrument with 1260 Quat Pump, 1260 Vialsampler, 1260 VWD. The sample was separated on a Waters Acquity BEH 200 Å SEC, 4.6 mm I.D.×15 cm, 1.7 μm column. SEC running buffer was PBS, pH 7.0, flowing at 0.40 ml/min. Absorbance was monitored at 214 nm, peak areas were manually integrated, and the percent of high molecular weight species (HMWS), low molecular weight species (LMWS), and monomer were reported. Results of SEC analysis following storage at 50° C. for 6 days (FIG. 32A) and 40° C. for 21 days (FIG. 32B) are shown. The slope of a linear regression of percentages of monomers are shown as a function of the pH of the specific buffers (FIG. 32C). These results demonstrated an optimal pH range from 6.5 to 7.0 for formulations including AB1310/AB1783-TriNKET®.

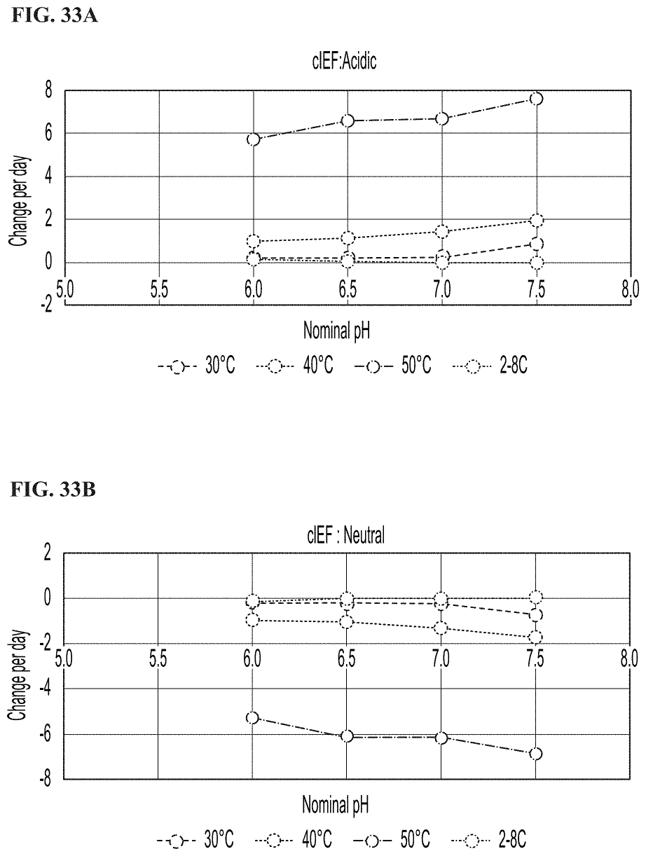

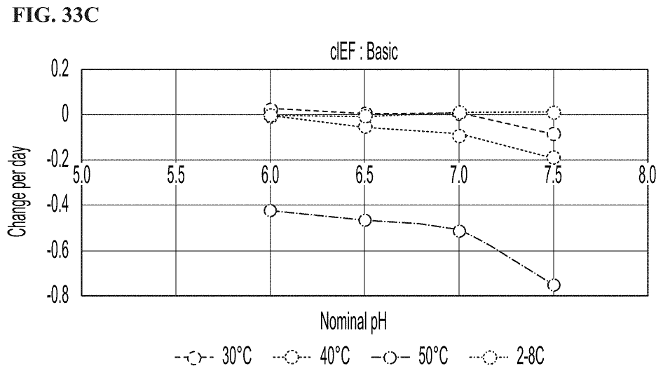

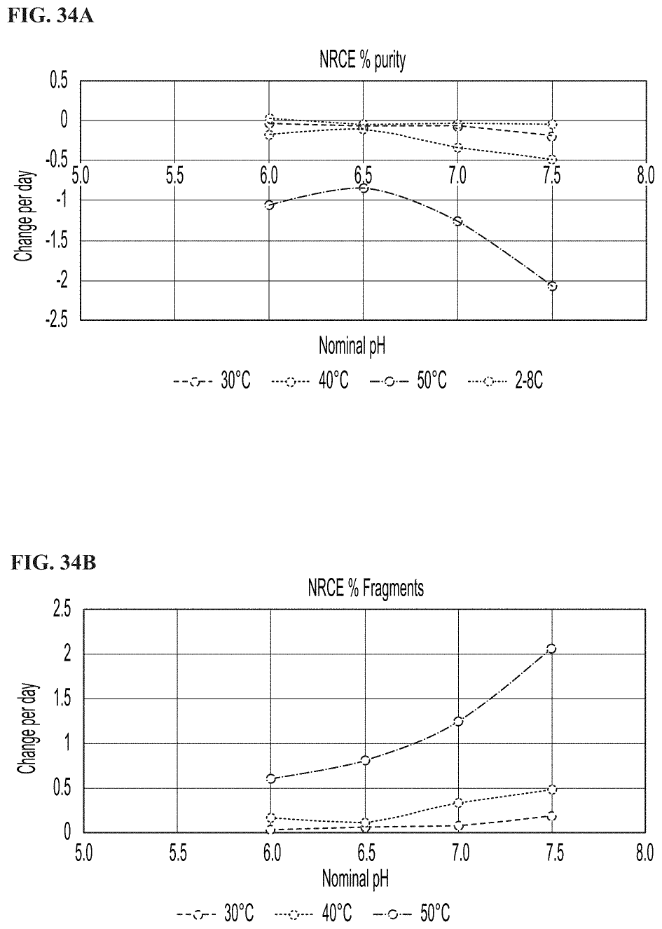

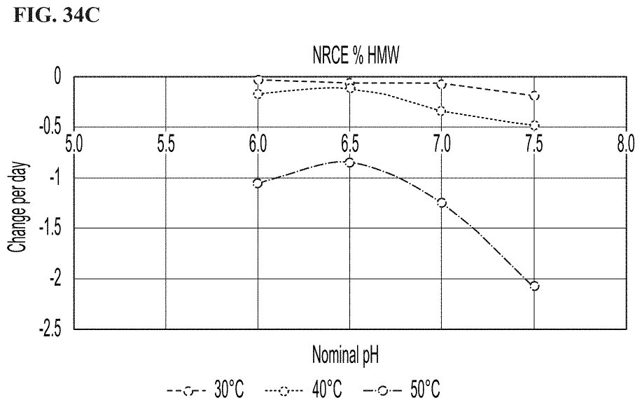

AB1310/AB1783-TriNKET® formulations were next characterized by capillary isoelectric focusing (cIEF) to assess charge profile. Changes in the acidic (FIG. 33A), neutral (FIG. 33B), and basic (FIG. 33C) regions of the electropherograms are shown. AB1310/AB1783-TriNKET® formulations were further examined by non-reducing capillary electrophoresis (NR-CE) to show purity of the AB1310/AB1783-TriNKET® (FIG. 34A), percentage of fragments (FIG. 34B), and formation of high molecular weight species (FIG. 34C). The combined results indicated an optimal pH range from 6.0 to 7.0.

### Excipient Screening

Formulations including different excipients and buffers were tested, including histidine or citrate buffers; sucrose, mannitol, or trehalose sugars; and effects of polysorbate 80. The compositions of tested buffers are shown in Table 29.

TABLE 29
Formulations Tested in Excipient Screening

| | AB1310/ | | | |
|---|---|---|---|---|
| | AB1783- | | | |
| Formulation | TriNKET ® | Buffer | Sugar | PS80 |
| B1 | 2 mg/mL | 20 mM | 6% Sucrose | 0.01% |
| | | Histidine | | |
| B2 | 2 mg/mL | 20 mM | 6% Sucrose | 0% |
| | | Histidine | | |
| B3 | 2 mg/mL | 20 mM Citrate | 6% Sucrose | 0.01% |
| B4 | 2 mg/mL | 20 mM Citrate | 6% Sucrose | 0% |
| B5 | 2 mg/mL | 20 mM Citrate | 4% Mannitol | 0.01% |
| B6 | 2 mg/mL | 20 mM Citrate | 6% Trehalose | 0.01% |
| B7 | 2 mg/mL | 20 mM Citrate | 5%/1% | 0.01% |
| | | | Sucrose/Mannitol | |

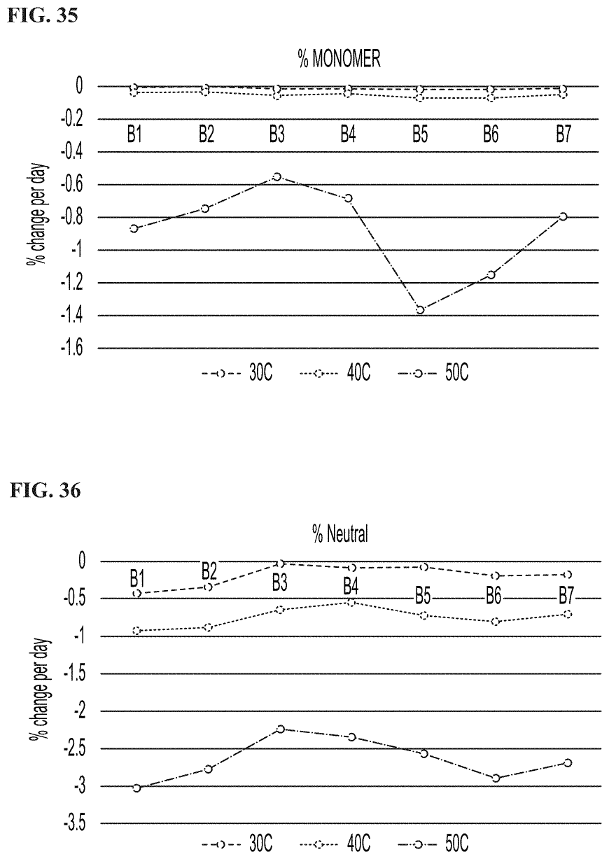

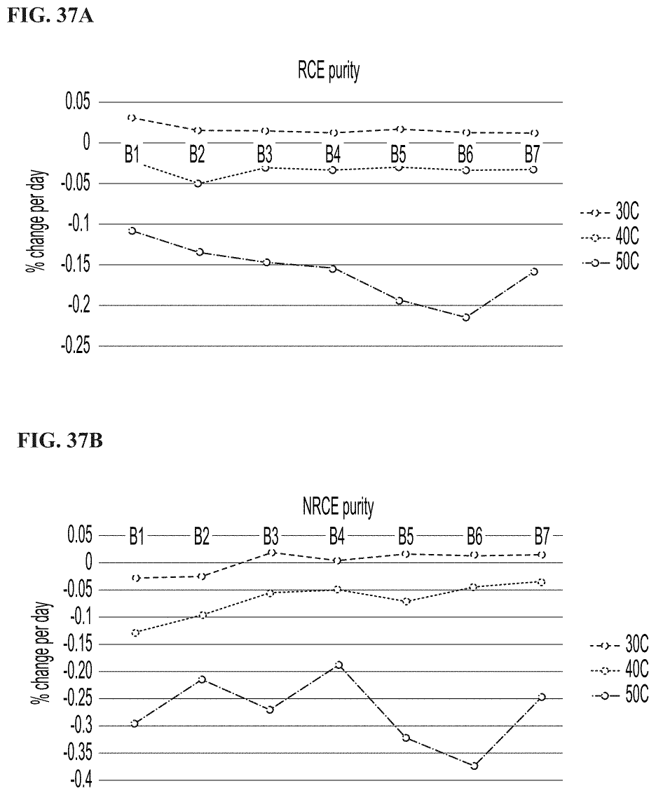

The various formulations were analyzed by SEC (FIG. 35), cIEF (FIG. 36), R-CE (FIG. 37A), and NR-CE (FIG. 37B). Results indicated that formulations containing citrate and sucrose conferred superior stability of the multispecific binding protein compared to formulations containing mannitol or trehalose instead of sucrose, or histidine buffer instead of citrate buffer.

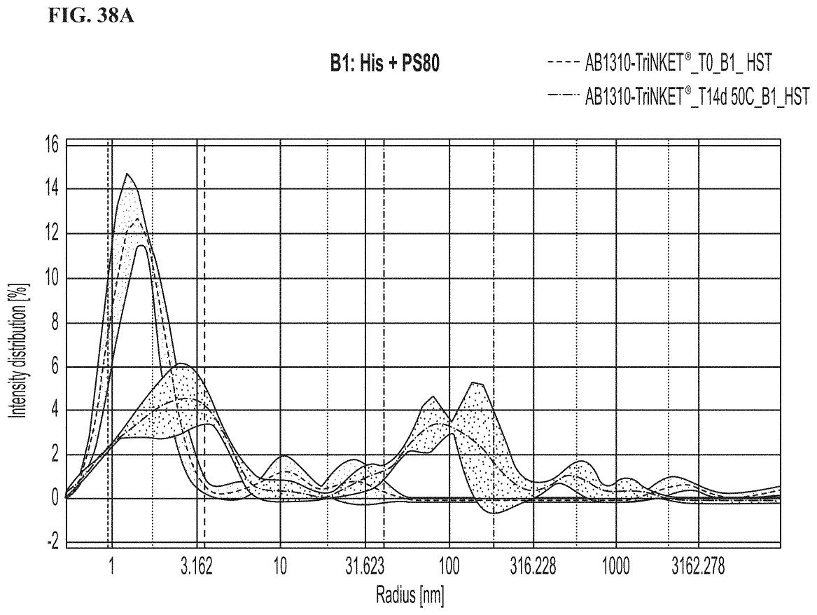

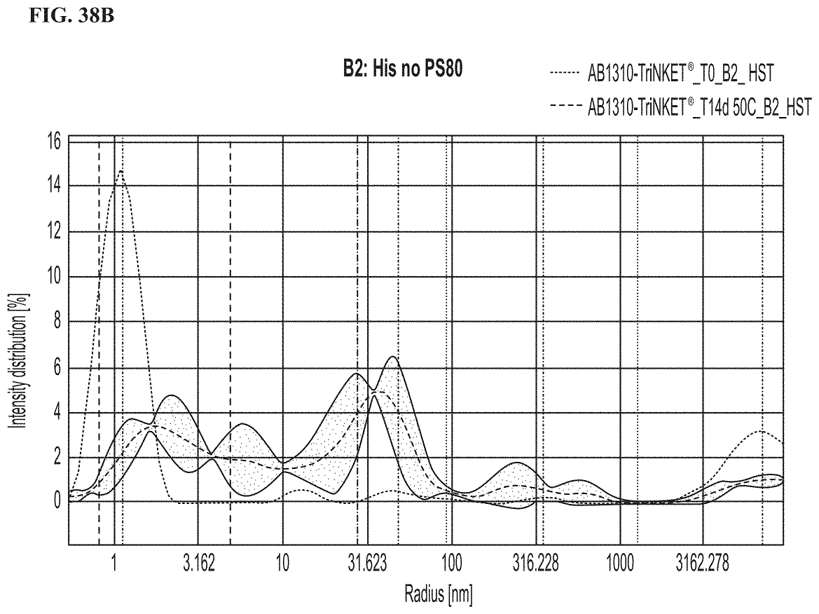

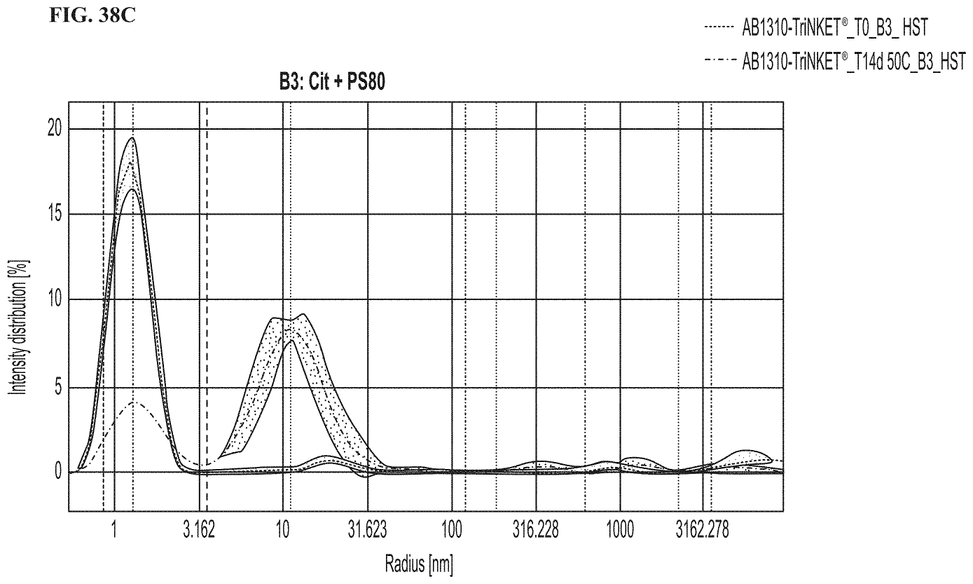

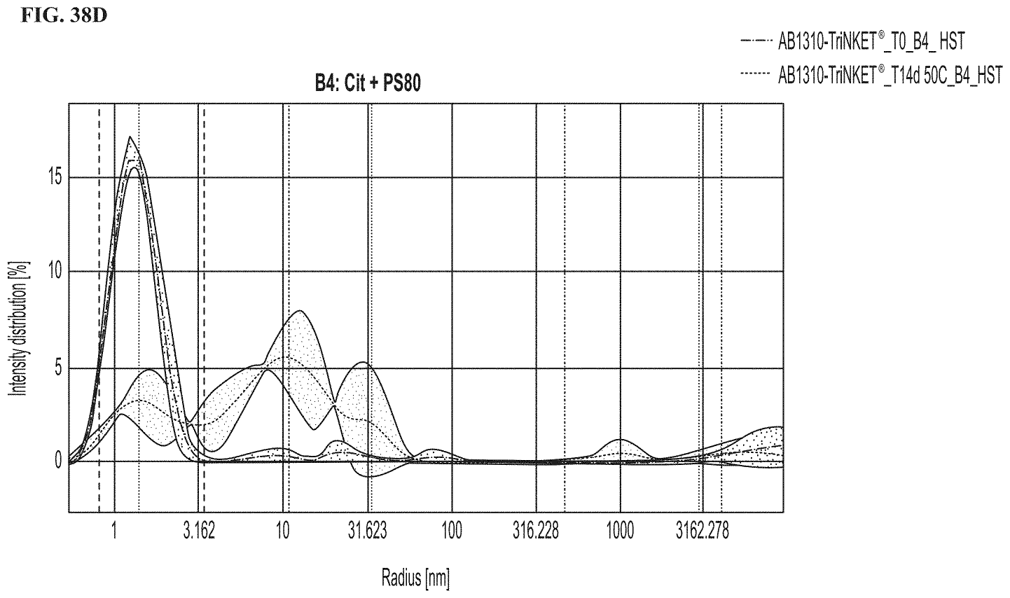

The self-interacting propensity of AB1310/AB1783-TriNKET® within the various formulations at baseline or temperature stress (50° C.) conditions was explored by dynamic light scattering (DLS). For DLS, briefly, k_{D} was determined using the Nanotemper Prometheus Panta, run in high sensitivity DLS mode. In short, samples were prepared in buffer and 10 L was loaded into three individual capillaries for analysis per concentration. The results were fit in the Panta Analysis software, and k_{D} values were calculated for each formulation individually. Tested formulations included: histidine buffer plus 0.01% polysorbate 80 (B1), histidine buffer with no polysorbate 8 (B2), citrate buffer with 0.01% polysorbate 80 (B3), and histidine buffer with no polysorbate 80 (B4). Results for the tested buffers are shown in FIGS. 38A-38D. Citrate-buffered formulations showed overall lower molecular weight and fewer total species compared to formulations including histidine buffers after incubation at 50° C.

### Range-Finding Study

Formulations of AB1310/AB1783-TriNKET® were tested to optimize the concentrations of sucrose and polysorbate 80. Tested formulations are shown in Table 30. Overall, there was no observed effect of polysorbate 80 on TriNKET® stability.

TABLE 30
Formulations Tested in Range-Finding Study

| AB1310/AB1783- | | | |
|---|---|---|---|
| TriNKET ® | Buffer | Sugar | PS80 |
| 15 mg/mL | 20 mM citrate, pH 6.5 | 6% Sucrose | 0.01% |
| 15 mg/mL | 20 mM citrate, pH 6.5 | 6% Sucrose | None |
| 15 mg/mL | 20 mM citrate, pH 6.5 | 6% Sucrose | 0.03% |
| 15 mg/mL | 20 mM citrate, pH 6.5 | 3% Sucrose | 0.01% |
| 15 mg/mL | 20 mM citrate, pH 6.5 | 9% Sucrose | 0.01% |

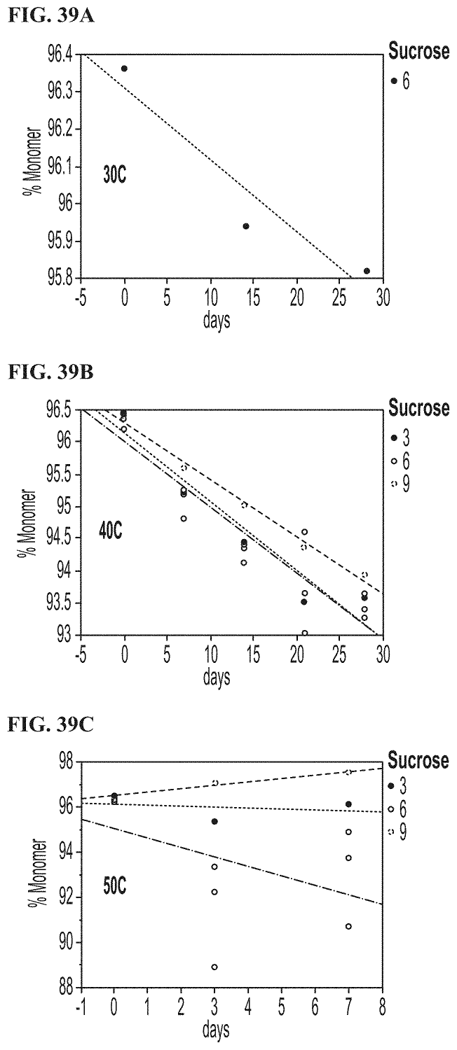

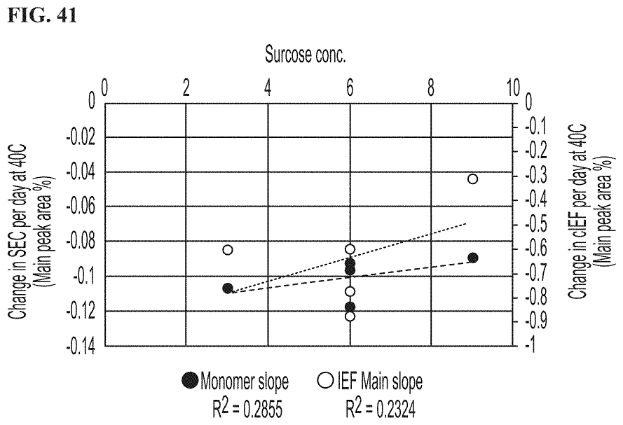

The various formulations were tested by SEC following 28 days of storage at 30° C. (FIG. 39A), 40° C. (FIG. 39B), and 50° C. (FIG. 39C) to observe the effects of sucrose concentration. After incubation at 40° C. a decrease in monomer from about 2.5% to 3.3% was observed across all formulations. The formulations were further analyzed by cIEF following 28 days of storage at 30° C. (FIG. 40A), 40° C. (FIG. 40B), and 50° C. (FIG. 40C) to observe the effects of sucrose concentration. Similar to the SEC results, a decrease in neutral species from about 9% to 24% was observed across all formulations after incubation at 40° C. Results for the two experiments are summarized in FIG. 41.

Overall, results suggested a formulation including 20 mM citrate buffer at pH 6.5 for buffering, 6% sucrose as tonicity modifier, and 0.01% polysorbate 80 as surfactant for AB1310/AB1783-TriNKET®.

### Example 4—CD8+ T Cell-Mediated Cytotoxicity Against 5T4+ Tumor Cells

This example shows that AB1310/AB1783-TriNKET® triggers tumor cell cytolysis by CD8+ T cells. The ability of AB1310/AB1783-TriNKET® to enhance CD8+ T cell mediated activity over multiple days in culture was investigated using IL-15 primed CD8+ T cells from 3 healthy donors for each of the two 5T4+ tumor cell lines and assessed using the INCUCYTE® Live-Cell Imager. AB1310/AB1783-TriNKET® stimulated dose-dependent increases in CD8+ T-cell lysis of 5T4-expressing tumor cells, while parental mAb showed no enhancement of CD8+ T-cell cytolysis over basal levels (FIG. 41; Table 30). A high concentration (50 nM) of TriNKET® control molecule that is unable to engage NKG2D (AB1310/AB1783-TriNKET®-NKG2Dsi) also failed to enhance lysis by CD8+ T cells.

### Methods

### Preparation of Reagents

Complete Roswell Park Memorial Institute (RPMI) primary cell culture media was prepared by adding 10% heat-inactivated fetal bovine serum (HI-FBS), 1× GlutaMAX™ 1× penicillin/streptomycin (Pen/Strep), and 50 μM of 2-mercaptoethanol (BME) to RPMI 1640 medium.

CD8+ T Cell Activation, Isolation, and Expansion from PBMCs

Frozen PBMCs were thawed and put into culture with 1-μg/mL Concanavalin A in RPMI primary cell culture media at 2.5×10⁶ cells/mL for 18 hours at 37° C. with 5% CO₂. Media was replaced, and cells were cultured with 5-ng/mL IL-2 for 2 days before subsequent replacement with 25-ng/mL IL-15 for 1 day. CD8+ T cells were purified using a negative selection technique with magnetic beads, according to manufacturer's instructions. Finally, CD8+ T cells were cultured in media containing 25-ng/mL IL-15 at 1×10⁶ cells/mL for 9 days, with media and cytokine replacement every 2 days, before use in the cytolysis assay.

### Long-Term CD8+ T Cell Cytotoxicity Incucyte® Assay

IL-15-activated CD8+ T cells were pelleted and resuspended in RPMI primary cell culture media at 2×10⁶ cells/mL, in preparation for a 20:1 effector-to-target cell ratio (E:T) and held at 37° C. with 5% CO₂ until ready for use. Human tumor cells stably expressing NucLight™ Green were rinsed gently with 1×phosphate-buffered saline (PBS), detached with TrypLE enzyme, pelleted, resuspended in complete RPMI primary cell culture media at 3×10⁴ cells/mL. Tumor cell suspension (100 μL) was distributed to each well of a sterile flat-bottom 96-well microplate. The plate was incubated at room temperature for 30 minutes before edge reservoirs were filled with 1.5 to 2.0 mL of 1×PBS. The plate was transferred to an incubator for a further 3.5-hour incubation at 37° C. with 5% CO₂. Dose titrations of 4× test articles were prepared in complete RPMI primary cell culture media, for final assay concentrations of AB1310/AB1783-TriNKET® and parental mAb ranging from 100 nM to 0.258 μM or 50 nM to 48.8 μM in 1:5 or 1:4 serial dilutions respectively. For AB1310/AB1783-TriNKET®-NKG2Dsi, either 1:5 serial dilutions were made ranging from 100 nM to 0.8 nM or a single concentration of 50 nM was used. The assay plate containing tumor cells was removed from incubator, and 50 μL of 4×test article dilutions and 50 μL of CD8+ T cell suspension were added to sample wells. RPMI primary cell culture media, and the CD8+ T cell suspension (50 μL each) were added to E:T-only, no treatment control wells, while tumor cell-only control wells simply received 100 μL of RPMI primary cell culture media. The plate was then transferred to an Incucyte® Live-Cell Imager for incubation at 37° C. with 5% CO₂ of at least 72 hours. Green fluorescent images were taken by the Incucyte® imager on a schedule of every 24 hours to assess the growth and survival of green tumor cells over time. Green tumor cells in each well counted by the Incucyte® software were normalized to counts from the same well from the initial timepoint (t0) to assess growth inhibition in a way that controlled for variability in cell seeding in the well imaging area. Each sample was run in duplicate or triplicate, and 2 images were taken per well, for 4 total images per sample condition at each time point. Tumor cell growth inhibition was interpreted as an indicator of CD8+ T cell-mediated activity stimulated by AB1310/AB1783-TriNKET®.

Percent (%) inhibition was calculated using the following formula:

% Inhibition=(T₇₂−S₇₂)/T₇₂*100

T₇₂: Average green count of tumor cell-only wells at 72 hours normalized to 0 hour time point
S₇₂: Average green count of sample well at 72 hours normalized to 0 hour time point

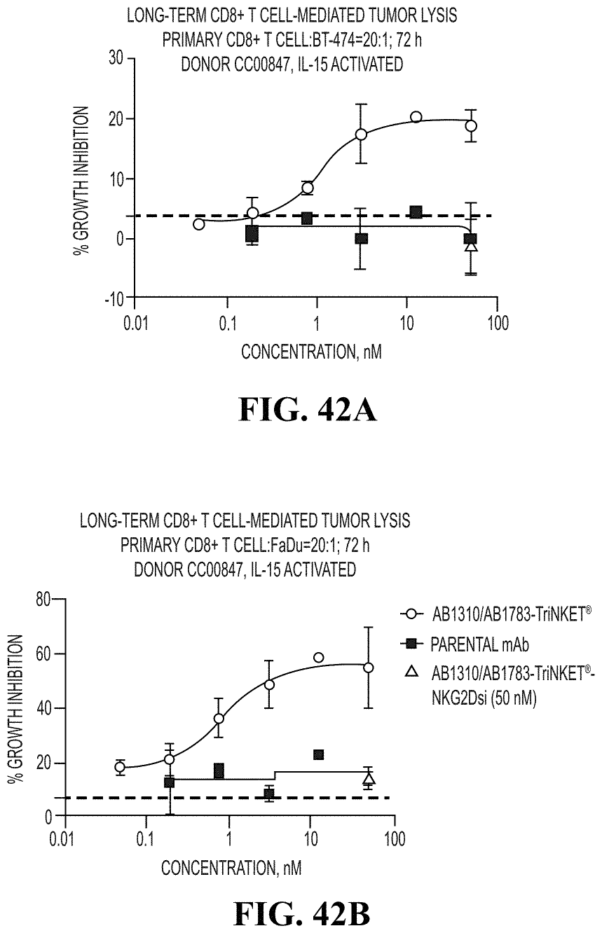

The % inhibition for each concentration was plotted against the test article concentration on a logarithmic axis, and the maximal enhancement of inhibition and the EC50 were calculated based on a 4-parameter nonlinear regression analysis using GraphPad Prism v9. The results are summarized in Table 31 and in FIG. 42.

TABLE 31
Summary of CD8+ T-Cell Lysis of 5T4+ Target
Cells by AB1310/AB1783-TriNKET ®

| | | | AB1310/AB1783 | | | |
|---|---|---|---|---|---|---|
| | | | TriNKET ® | | Parental mAb | |
| | | | | Max | | Max |
| | | Average | | enhanced | | enhanced |
| Cell | Origin; | 5T4 per | EC₅₀ | inhibition | EC₅₀ | inhibition |
| Line | Details | Cell | (nM) | (%) | (nM) | (%) |
| BT-474 | BRCA | 10,000 | 0.66 ± 0.43 | 24 ± 6 | NR | NR |
| FaDu | HNSCC | 13,000 | 0.59 ± 0.37 | 26 ± 12 | NR | NR |

BRCC: breast cancer; HNSCC: head and neck squamous cell carcinoma; E:T: effector-to-target ratio; EC50: concentration resulting in half-maximal response (based on 4-parameter, nonlinear regression curve fit); Max: maximal;
Note:
Mean ± standard deviation (SD) values were calculated using n number of healthy PBMC donors. NR: not recorded. Each sample was run in either duplicate or triplicate.

NKG2D is expressed on cytotoxic CD8+ T cells in addition to NK cells, and can CD8+ T cells be triggered directly by NKG2D stimulation when activated via its TCR and/or cytokines. The ability of AB1310/AB1783-TriNKET® to induce killing of 5T4-expressing tumor cells via cytokine-stimulated CD8+ T cells was assessed using in vitro-expanded, IL-15-primed CD8+ T cells co-cultured with 5T4-expressing tumor cells. AB1310/AB1783-TriNKET® enhanced the lysis of 5T4+ tumor cells by CD8+ T cells, which had been expanded and primed with IL-15, in a dose-responsive fashion. Parental mAb had no activity in this context, consistent with the lack of NKG2D engagement. In addition, AB1310/AB1783-TriNKET®-NKG2Dsi was unable to increase CD8+ T-cell lysis, confirming that AB1310/AB1783-TriNKET® triggers CD8+ T cells directly by NKG2D stimulation.

### Example 5—AB1310/AB1783-TriNKET® Stimulates 5T4-Dependent NK Cell-Mediated Cytotoxicity

This example shows the lytic activity of AB1310/AB1783-TriNKET® in long-term (˜72 hours) co-culture assays using primary human NK cells with 5T4+ human cancer cell lines or 5T4+ cancer associated fibroblasts (CAFs).

The ability of AB1310/AB1783-TriNKET® to enhance NK cell activity over multiple days in culture was investigated using overnight-rested primary human NK cells from 6 healthy donors on a panel of 4 5T4+ tumor cell lines and assessed using the IncuCyte® Live-Cell Imager. Donors expressing the high-affinity CD16a variant V158 (heterozygous (V/F)), as well as those with only the low-affinity CD16a variant (F/F), were represented among the donors used to characterize the activity of AB1310/AB1783-TriNKET®.

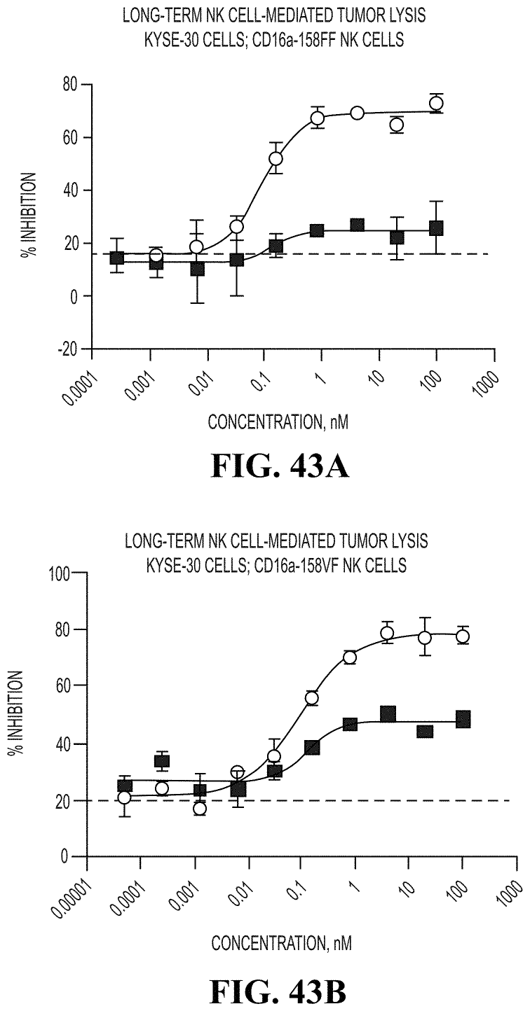

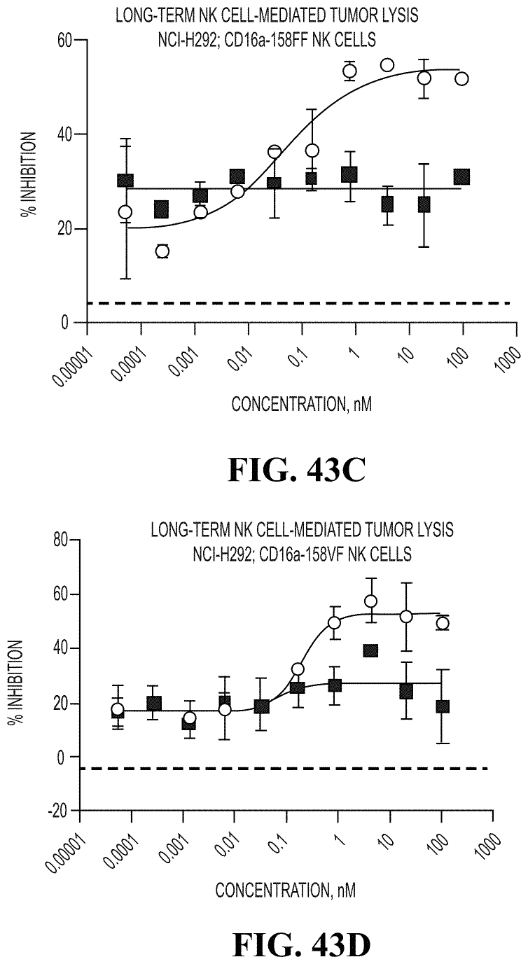

The robust NK cell-mediated lysis of tumor cells triggered by AB1310/AB1783-TriNKET® in short-term (2.5-hour) assays translated into substantial inhibition of 5T4+ tumor cell survival and growth detected by IncuCyte® imaging over a 3-day culture period (FIG. 43). Tumor cell lines representing indications of potential clinical interest were tested. AB1310/AB1783-TriNKET® demonstrated subnanomolar potency and enhanced maximal inhibition from 26% to 51% above basal inhibition mediated by NK cells against tumor cells alone (Table 32). Maximal inhibition achieved by AB1310/AB1783-TriNKET® exceeded that of parental mAb (2% to 15% above E:T background) for both CD16a V/F and F/F donors against each cell line.

### Methods

### Long-Term Cytotoxicity Incucyte® Assay

Thawed frozen human NK cells from 6 healthy human donors were put into culture at 10⁶ cells/mL in RPMI primary cell culture media overnight. Human tumor cells stably expressing NucLight™ Green were rinsed gently with 1×phosphate-buffered saline (PBS), detached with TrypLE enzyme, pelleted, resuspended in complete RPMI primary cell culture media at 3×10⁴ cells/mL. Tumor cell suspension (100 μL) was distributed to each well of a sterile flat-bottom 96-well microplate. The plate was incubated at room temperature for 30 minutes before edge reservoirs were filled with 1.5 to 2.0 mL of 1×PBS. The plate was transferred to an incubator for a further 3.5-hour incubation at 37° C. with 5% CO₂. Dose titrations of 4× test articles were prepared using 1:5 serial dilutions in complete RPMI primary cell culture media, for final assay concentrations of AB1310/AB1783-TriNKET® and parental mAb ranging from 100 nM to 0.25 μM or 100 nM to 0.05 μM or 100 nM to 1.2 pM. Rested NK cells were recovered from culture, pelleted, resuspended in complete RPMI primary cell culture media at 3×10⁵ cells/mL in preparation for a 5:1 effector to target ratio (E:T).

The assay plate containing tumor cells was removed from incubator, and 50 μL of 4× test article dilutions and 50 μL of NK cell suspension were added to sample wells. RPMI primary cell culture media, and the NK cell suspension (50 μL each) were added to E:T-only, no-treatment control wells, while tumor cell-only control wells simply received 100 μL of RPMI primary cell culture media. The plate was then transferred to an Incucyte® Live-Cell Imager for incubation at 37° C. with 5% CO₂ of at least 72 hours. Green fluorescent images were taken by the Incucyte® imager on a schedule of every 24 hours to assess the growth and survival of green tumor cells over time. Green tumor cells in each well counted by the Incucyte® software were normalized to counts from the same well from the initial timepoint (to) to assess growth inhibition in a way that controlled for variability in cell seeding in the well imaging area. Each sample was run in duplicate, and 2 images were taken per well, for 4 total images per sample condition at each time point. Tumor cell growth inhibition was interpreted as an indicator of NK cell-mediated activity stimulated by AB1310/AB1783-TriNKET® and parental mAb.

Percent (%) inhibition was calculated using the following formula:

% Inhibition=(T72−S72)/T72*100

T72: Average green count of tumor cell-only wells at 72 hours normalized to 0 hour time point
S72: Average green count of sample well at 72 hours normalized to 0 hour time point

The % inhibition for each concentration was plotted against the test article concentration on a logarithmic axis, and the maximal enhancement of inhibition and the EC₅₀ were calculated based on a 4-parameter nonlinear regression analysis using GraphPad Prism v9. The results are summarized in Table 32 and in FIG. 43.

TABLE 32
Summary of Long-Term NK Cell-Mediated Cytotoxicity of AB1310/AB1783-
TriNKET ® on 5T4+ Tumor Cell Lines

| | | AB1310/AB1783-TriNKET ® | | | Parental mAb | |
|---|---|---|---|---|---|---|
| | | | | Max | | Max |
| NucLight ™ | Average | | | Enhanced | | Enhanced |
| Green Cell | 5T4 per | EC50 | EC50 | Inhibition | EC50 | Inhibition |
| Line | Cell | (nM) | (nM) | (%) | (nM) | (%) |
| KYSE-30 | 51,000 | 0.087 ± 0.016 | 0.87 ± 0.86 | 51 ± 9 | 0.13 ± 0.01^{a} | 15 ± 5 |
| HCT 116 | 10,000 | 0.16 ± 0.07 | 0.37 ± 0.43 | 26 ± 8 | 0.0025³ | 11 ± 11 |
| NCI-H292 | 8,000 | 0.37 ± 0.43 | 2.9 ± 3.6 | 32 ± 5 | NR | 6.6 ± 5.6 |
| HT-29 | 4,000 | 1.3 ± 0.4 | 19 ± 25 | 32 ± 16 | NR | 2.3 ± 1.5 |

EC50: concentration resulting in half-maximal response (based on 4parameter, nonlinear regression curve fit); EC90: concentration resulting in 90% of maximal response (based on 4-parameter, nonlinear regression curve fit); Max: maximal; NR: not recorded.
Mean ± standard deviation (SD) values were calculated using an n as noted as the number of separate healthy NK cell donors with either a V/F or F/F CD16a genotype evaluated for each cell line. Each sample was run in duplicate.
^{a}Average EC50 value for 1 or 2 out of 3 donors because of a low response of the second or third donor, which could not be confidently fit to curve.

The robust NK cell-mediated lysis of tumor cells triggered by AB1310/AB1783-TriNKET® in short-term assays translated into substantial inhibition of 5T4-expressing tumor cell outgrowth. AB1310/AB1783-TriNKET® stimulated robust maximal inhibition of tumor cells with potency ranging from 0.087 to 1.322 nM. Additionally, AB1310/AB1783-TriNKET® demonstrated superior maximal tumor inhibition when compared to parental mAb, across 6 healthy human NK cell donors with either V/F or F/F CD16a genotype.

APPENDIX
Exemplary Multispecific Binding Protein Sequences
AB2092 - (hF3′.A49-M-I-YA.5T4-05H04-VH02_BM1-VL_BM1-
VH-LH-SS-scFv-YB)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTESSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
ENVLTQSPATLSLSPGERATLSCSAKSSVSYIHWYQQKPGQAPRLLIYDTSYLGSGIPARES
GSGSGTDYTLTISSLEPEDFAVYYCQQWSSYPYTFGCGTKVEIKGGGGSGGGGSGGGGSGGG
GSQVQLVQSGAEVKKPGASVKVSCKASGYKFTDYYMDWVRQAPGQCLEWIGYIFPNDASTTY
NEKFKGKATLTADKSTNTAYMELSSLRSEDTAVYYCARSRDADYWGQGTTVTVSSGSDKTHT
CPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPRVY
TLPPCRDELTKNQVSLTCLVKGFYPSDIAVEWESNGQPENNYKTTPPVLVSDGSFTLYSKLT
VDKSRWQQGNVFSCSVMHEALHNHYTQKSLSLSPG (SEQ ID NO: 220)
AB2093 - hF3′.A49-M-I-YA.5T4-05H04-VH02_BM1-VL_BM1-
VH-HL-SS-scFv-YB
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTESSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
QVQLVQSGAEVKKPGASVKVSCKASGYKFTDYYMDWVRQAPGQCLEWIGYIFPNDASTTYNE
KFKGKATLTADKSTNTAYMELSSLRSEDTAVYYCARSRDADYWGQGTTVTVSSGGGGSGGGG
SGGGGSGGGGSENVLTQSPATLSLSPGERATLSCSAKSSVSYIHWYQQKPGQAPRLLIYDTS
YLGSGIPARFSGSGSGTDYTLTISSLEPEDFAVYYCQQWSSYPYTFGCGTKVEIKGSDKTHT
CPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPRVY
TLPPCRDELTKNQVSLTCLVKGFYPSDIAVEWESNGQPENNYKTTPPVLVSDGSFTLYSKLT
VDKSRWQQGNVFSCSVMHEALHNHYTQKSLSLSPG (SEQ ID NO: 222)
AB2143 - hF3′.A49-M-I-YA.5T4-O5HO4-VHO2_BM1-VL_BM1-
VH-Q1E-HL-SS-scFv-YB
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTESSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
EVQLVQSGAEVKKPGASVKVSCKASGYKFTDYYMDWVRQAPGQCLEWIGYIFPNDASTTYNE
KFKGKATLTADKSTNTAYMELSSLRSEDTAVYYCARSRDADYWGQGTTVTVSSGGGGSGGGG
SGGGGSGGGGSENVLTQSPATLSLSPGERATLSCSAKSSVSYIHWYQQKPGQAPRLLIYDTS
YLGSGIPARFSGSGSGTDYTLTISSLEPEDFAVYYCQQWSSYPYTFGCGTKVEIKGSDKTHT
CPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPRVY
TLPPCRDELTKNQVSLTCLVKGFYPSDIAVEWESNGQPENNYKTTPPVLVSDGSFTLYSKLT
VDKSRWQQGNVFSCSVMHEALHNHYTQKSLSLSPG (SEQ ID NO: 223)
AB2509 - hF3.5T4-05H04-VH02_BM1-VH-YA.A49M-I-
scFvSSFc-YB
Chain L:
ENVLTQSPATLSLSPGERATLSCSAKSSVSYIHWYQQKPGQAPRLLIYDTSYLGSGIPARES
GSGSGTDYTLTISSLEPEDFAVYYCQQWSSYPYTFGGGTKVEIKRTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC (SEQ ID NO: 254)
Chain H:
QVQLVQSGAEVKKPGASVKVSCKASGYKFTDYYMDWVRQAPGQGLEWIGYIFPNDASTTYNE
KFKGKATLTADKSTNTAYMELSSLRSEDTAVYYCARSRDADYWGQGTTVTVSSASTKGPSVF
PLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVCTLPPSRDELTENQVSLTCLVKGFYP
SDIAVEWESNGQPENNYKTTPPVLDSDGSFELYSWLTVDKSRWQQGNVFSCSVMHEALHNHY
TQKSLSLSPG (SEQ ID NO: 253)
Chain S:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGCGTKVEIKGGGGSGGGGSGGGGSGG
GGSEVQLVESGGGLVKPGGSLRLSCAASGFTFSSYSMNWVRQAPGKCLEWVSSISSSSSYIY
YADSVKGRFTISRDNAKNSLYLQMNSLRAEDTAVYYCARGAPIGAAAGWFDPWGQGTLVTVS
SGSDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKG
QPREPRVYTLPPCRDELTKNQVSLTCLVKGFYPSDIAVEWESNGQPENNYKTTPPVLVSDGS
FTLYSKLTVDKSRWQQGNVFSCSVMHEALHNHYTQKSLSLSPG (SEQ ID NO: 252)
AB2511 - hF3.5T4-11F09-5T4.C01-VH_BM2-VK_BM1-
YA.A49M-I-scFvSSFc-YB
Chain L:
DIQLTQSPSSLSASVGDRVTITCRASQGVSTSTYTYMHWFQQKPGKAPKLLIKFASNLESGV
PSRFSGSGSGTDFTLTISSLQPEDFATYYCQHSWEIPWTFGGGTKVEIKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGEC (SEQ ID NO: 256)
Chain H:
EVQLVESGGGLVKPGGSLRLSCEASGFTFSDYGMHWVRQAPGKGLEWLAYISSGSSTFYYAD
TVKGRFTISRDNAKNSLYLQMNSLRAEDTAVYYCGSSQSYYRGTMDYWGQGTTVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVCTLPPSRDELTENQVSLTCLV
KGEYPSDIAVEWESNGQPENNYKTTPPVLDSDGSFFLYSWLTVDKSRWQQGNVFSCSVMHEA
LHNHYTQKSLSLSPG (SEQ ID NO: 255)
Chain S:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGCGTKVEIKGGGGSGGGGSGGGGSGG
GGSEVQLVESGGGLVKPGGSLRLSCAASGFTFSSYSMNWVRQAPGKCLEWVSSISSSSSYIY
YADSVKGRFTISRDNAKNSLYLQMNSLRAEDTAVYYCARGAPIGAAAGWFDPWGQGTLVTVS
SGSDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKG
QPREPRVYTLPPCRDELTKNQVSLTCLVKGFYPSDIAVEWESNGQPENNYKTTPPVLVSDGS
FTLYSKLTVDKSRWQQGNVFSCSVMHEALHNHYTQKSLSLSPG (SEQ ID NO: 252)
AB2512 - hF3.5T4-11F09-5T4.C01-VH_BM2-VK_BM1_M33L-
YA.A49M-I-scFvSSFc-YB
Chain L:
DIQLTQSPSSLSASVGDRVTITCRASQGVSTSTYTYLHWFQQKPGKAPKLLIKFASNLESGV
PSRFSGSGSGTDFTLTISSLQPEDFATYYCQHSWEIPWTFGGGTKVEIKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGEC (SEQ ID NO: 258)
Chain H:
EVQLVESGGGLVKPGGSLRLSCEASGFTFSDYGMHWVRQAPGKGLEWLAYISSGSSTFYYAD
TVKGRFTISRDNAKNSLYLQMNSLRAEDTAVYYCGSSQSYYRGTMDYWGQGTTVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVCTLPPSRDELTENQVSLTCLV
KGFYPSDIAVEWESNGQPENNYKTTPPVLDSDGSFFLYSWLTVDKSRWQQGNVFSCSVMHEA
LHNHYTQKSLSLSPG (SEQ ID NO: 257)
Chain S:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGCGTKVEIKGGGGSGGGGSGGGGSGG
GGSEVQLVESGGGLVKPGGSLRLSCAASGFTFSSYSMNWVRQAPGKCLEWVSSISSSSSYIY
YADSVKGRFTISRDNAKNSLYLQMNSLRAEDTAVYYCARGAPIGAAAGWFDPWGQGTLVTVS
SGSDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKG
QPREPRVYTLPPCRDELTKNQVSLTCLVKGFYPSDIAVEWESNGQPENNYKTTPPVLVSDGS
FTLYSKLTVDKSRWQQGNVFSCSVMHEALHNHYTQKSLSLSPG (SEQ ID NO: 252)
AB2513 - hF3.5T4-10F10-5T4.C01-VH01_BM1-VL02_BM1-
YA.A49M-I-scFvSSFc-YB
Chain L:
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGGGTKVEIKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGEC (SEQ ID NO: 260)
Chain H:
EVQLVESGGGLVKPGGSLRLSCAASRFTFSDFGMHWVRQAPGKGLEWVSYISSGSSTIYYAD
TVKGRFTISRDNAKNSLYLQMNSLRAEDTAVYYCASSQSYYRGTLDYWGQGTTVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVCTLPPSRDELTENQVSLTCLV
KGFYPSDIAVEWESNGQPENNYKTTPPVLDSDGSFFLYSWLTVDKSRWQQGNVFSCSVMHEA
LHNHYTQKSLSLSPG (SEQ ID NO: 259)
Chain S:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGCGTKVEIKGGGGSGGGGSGGGGSGG
GGSEVQLVESGGGLVKPGGSLRLSCAASGFTFSSYSMNWVRQAPGKCLEWVSSISSSSSYIY
YADSVKGRFTISRDNAKNSLYLQMNSLRAEDTAVYYCARGAPIGAAAGWFDPWGQGTLVTVS
SGSDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKG
QPREPRVYTLPPCRDELTKNQVSLTCLVKGFYPSDIAVEWESNGQPENNYKTTPPVLVSDGS
FTLYSKLTVDKSRWQQGNVFSCSVMHEALHNHYTQKSLSLSPG (SEQ ID NO: 252)
AB2514 - hF3.5T4-10F10-5T4.C01-VH01_BM1_T62S-
VL02_BM1-YA.A49M-I-scFvSSFc-YB
Chain L:
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGGGTKVEIKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGEC (SEQ ID NO: 262)
Chain H:
EVQLVESGGGLVKPGGSLRLSCAASRFTFSDFGMHWVRQAPGKGLEWVSYISSGSSTIYYAD
SVKGRFTISRDNAKNSLYLQMNSLRAEDTAVYYCASSQSYYRGTLDYWGQGTTVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVCTLPPSRDELTENQVSLTCLV
KGFYPSDIAVEWESNGQPENNYKTTPPVLDSDGSFFLYSWLTVDKSRWQQGNVFSCSVMHEA
LHNHYTQKSLSLSPG (SEQ ID NO: 261)
Chain S:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGCGTKVEIKGGGGSGGGGSGGGGSGG
GGSEVQLVESGGGLVKPGGSLRLSCAASGFTFSSYSMNWVRQAPGKCLEWVSSISSSSSYIY
YADSVKGRFTISRDNAKNSLYLQMNSLRAEDTAVYYCARGAPIGAAAGWFDPWGQGTLVTVS
SGSDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKG
QPREPRVYTLPPCRDELTKNQVSLTCLVKGFYPSDIAVEWESNGQPENNYKTTPPVLVSDGS
FTLYSKLTVDKSRWQQGNVFSCSVMHEALHNHYTQKSLSLSPG (SEQ ID NO: 252)
AB1878 - hF3′.A49-M-I-YA.5T4-10F10-IGHV3-
21*05-BM1-YB (alternative humanization of
clone 10E10)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTFSSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLVESGGGLVKPGGSLRLSCAASRFTFSDFGMHWVRQAPGKCLEWVSYISSGS
STIYYADTVKGRFTISRDNAKNSLYLQMNSLRAEDTAVYYCASSQSYYRGTLDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 224)
AB1881 - hF3′.A49-M-I-YA.5T4-10F10-IGHV3-
23*03-BM2-YB (alternative humanization of
clone 10F10)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTFSSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLLESGGGLVQPGGSLRLSCAASRFTFSDFGMHWVRQAPGKCLEWIAYISSGS
STIYYADTVKGRFTISRDNAKNTLYLQMNSLRAEDTAVYYCASSQSYYRGTLDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 225)
AB1882 - hF3′.A49-M-I-YA.5T4-10F10-IGHV3-
48*01-BM1-YB (alternative humanization of
clone 10F10)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTFSSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLVESGGGLVQPGGSLRLSCAASRFTFSDFGMHWVRQAPGKCLEWVSYISSGS
STIYYADTVKGRFTISRDNAKNSLYLQMNSLRAEDTAVYYCASSQSYYRGTLDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 226)
AB1884 - hF3′.A49-M-I-YA.5T4-10F10-IGHV3-
48*01-BM2-YB (alternative humanization of
clone 10F10)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTFSSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLVESGGGLVQPGGSLRLSCAASRFTFSDFGMHWVRQAPGKCLEWIAYISSGS
STIYYADTVKGRFTISRDNAKNSLYLQMNSLRAEDTAVYYCASSQSYYRGTLDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 227)
AB1885 - hF3′.A49-M-I-YA.5T4-10F10-IGHV3-
11*01-BM2-YB (alternative humanization of
clone 10F10)
Chain L:
DIQMTQSPSSVSASVGDRVTITCRASQGISSWLAWYQQKPGKAPKLLIYAASSLQSGVPSRE
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTESSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSQVQLVESGGGLVKPGGSLRLSCAASRFTFSDFGMHWVRQAPGKCLEWIAYISSGS
STIYYADTVKGRFTISRDNAKNSLYLQMNSLRAEDTAVYYCASSQSYYRGTLDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 230)
AB1886 - hF3′.A49-M-I-YA.5T4-10F10-IGHV3-
11*01-BM1-YB (alternative humanization of
clone 10E10)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTESSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLVESGGGLVKPGGSLRLSCAASRFTFSDFGMHWIRQAPGKCLEWVSYISSGS
STIYYADTVKGRFTISRDNAKNSLYLQMNSLRAEDTAVYYCASSQSYYRGTLDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 231)
AB1887 - hF3′.A49-M-I-YA.5T4-10F10-IGHV3-
21*05-BM2-YB (alternative humanization of
clone 10F10)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTESSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLVESGGGLVKPGGSLRLSCAASRFTFSDFGMHWVRQAPGKCLEWIAYISSGS
STIYYADTVKGRFTISRDNAKNSLYLQMNSLRAEDTAVYYCASSQSYYRGTLDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 234)
AB1892 - hF3′.A49-M-I-YA.5T4-10F10-IGHV3-
23*03-BM1-YB (alternative humanization of
clone 10E10)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTFSSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQSVTTSNYNYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLLESGGGLVQPGGSLRLSCAASRFTFSDFGMHWVRQAPGKCLEWVSYISSGS
STIYYADTVKGRFTISRDNSKNTLYLQMNSLRAEDTAVYYCASSQSYYRGTLDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 235)
AB1319 - hF3′.A49-M-I-YA.5T4-11F09-5T4.C01-
VH_BM2-VK_BM1-LH-scFv-SS-Fc-LH-YB
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTFSSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQGVSTSTYTYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLVESGGGLVKPGGSLRLSCEASGFTFSDYGMHWVRQAPGKCLEWLAYISSGS
STFYYADTVKGRFTISRDNAKNSLYLQMNSLRAEDTAVYYCGSSQSYYRGTMDYWGQGTTVT
VSSASDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 237)
AB1879 - hF3′.A49-M-I-YA.5T4-11F09-IGHV3-
48*01-BM1-YB (alternative humanization of
clone 11F09)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTFSSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQGVSTSTYTYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLVESGGGLVQPGGSLRLSCAASGFTFSDYGMHWVRQAPGKCLEWVSYISSGS
STFYYADTVKGRFTISRDNAKNSLYLQMNSLRAEDTAVYYCGSSQSYYRGTMDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 238)
AB1880 - hF3′.A49-M-I-YA.5T4-11F09-IGHV3-
21*05-BM2-YB (alternative humanization of
clone 11F09)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTESSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQGVSTSTYTYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLVESGGGLVKPGGSLRLSCEASGFTFSDYGMHWVRQAPGKCLEWLAYISSGS
STFYYADTVKGRFTISRDNAKNSLYLQMNSLRAEDTAVYYCGSSQSYYRGTMDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 239)
AB1883 - hF3′.A49-M-I-YA.5T4-11F09-IGHV3-
11*01-BM2-YB (alternative humanization of
clone 11F09)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNEYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTESSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQGVSTSTYTYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSQVQLVESGGGLVKPGGSLRLSCAASGFTFSDYGMHWVRQAPGKCLEWLAYISSGS
STFYYADTVKGRFTISRDNAKNSLYLQMNSLRAEDTAVYYCGSSQSYYRGTMDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 240)
AB1888 - hF3′.A49-M-I-YA.5T4-11F09-IGHV3-
21*05-BM1-YB (alternative humanization of
clone 11F09)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTESSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQGVSTSTYTYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLVESGGGLVKPGGSLRLSCAASGFTFSDYGMHWVRQAPGKCLEWVSYISSGS
STFYYADTVKGRFTISRDNAKNSLYLQMNSLRAEDTAVYYCGSSQSYYRGTMDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 241)
AB1889 - hF3′.A49-M-I-YA.5T4-11F09-IGHV3-
48*01-BM2-YB (alternative humanization of
clone 11F09)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTFSSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQGVSTSTYTYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLVESGGGLVQPGGSLRLSCAASGFTFSDYGMHWVRQAPGKCLEWLAYISSGS
STFYYADTVKGRFTISRDNAKNSLYLQMNSLRAEDTAVYYCGSSQSYYRGTMDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 244)
AB1890 - hF3′.A49-M-I-YA.5T4-11F09-IGHV3-
23*03-BM1-YB (alternative humanization of
clone 11F09)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTFSSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQGVSTSTYTYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLLESGGGLVQPGGSLRLSCAASGFTFSDYGMHWVRQAPGKCLEWVSYISSGS
STFYYADTVKGRFTISRDNSKNTLYLQMNSLRAEDTAVYYCGSSQSYYRGTMDYWGQGTTVT
VSSGSDKTHTCPPCPAPELLGGPSVELEPPKPKDTLMISRTPEVTCVVVDVSHEDPEVKENW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 286)
AB1891 - hF3′.A49-M-I-YA.5T4-11F09-IGHV3-
11*01-BM1-YB (alternative humanization of
clone 11F09)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTFSSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQGVSTSTYTYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLVESGGGLVKPGGSLRLSCAASGFTFSDYGMHWIRQAPGKCLEWVSYISSGS
STFYYADTVKGRFTISRDNAKNSLYLQMNSLRAEDTAVYYCGSSQSYYRGTMDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 287)
AB1893 - hF3′.A49-M-I-YA.5T4-11F09-IGHV3-
23*03-BM2-YB (alternative humanization of
clone 11F09)
Chain L:
DIQMTQSPSSVSASVGDRVTITCRASQGISSWLAWYQQKPGKAPKLLIYAASSLQSGVPSRF
SGSGSGTDFTLTISSLQPEDFATYYCQQGVSFPRTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC (SEQ ID NO: 200)
Chain H:
EVQLVESGGGLVKPGGSLRLSCAASGFTESSYSMNWVRQAPGKGLEWVSSISSSSSYIYYAD
SVKGRFTISRDNAKNSLYLQMNSLRAEDTAVYYCARGAPIGAAAGWFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRDELTENQVSLTC
LVKGFYPSDIAVEWESNGQPENNYKTTPPVLDSDGSFFLYSWLTVDKSRWQQGNVFSCSVMH
EALHNHYTQKSLSLSPG (SEQ ID NO: 199)
Chain S:
DIQLTQSPSSLSASVGDRVTITCRASQGVSTSTYTYMHWFQQKPGKAPKLLIKFASNLESGV
PSRFSGSGSGTDFTLTISSLQPEDFATYYCQHSWEIPWTFGCGTKVEIKGGGGSGGGGSGGG
GSGGGGSEVQLLESGGGLVQPGGSLRLSCAASGFTFSDYGMHWVRQAPGKCLEWLAYISSGS
STFYYADTVKGRFTISRDNAKNTLYLQMNSLRAEDTAVYYCGSSQSYYRGTMDYWGQGTTVT
VSSGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPRVYTLPPCRDELTKNQVSLTCLVKGFYPSDIAVEWESNGQPENNYKTTPPVLVSD
GSFTLYSKLTVDKSRWQQGNVFSCSVMHEALHNHYTQKSLSLSPG (SEQ ID NO: 249)

### INCORPORATION BY REFERENCE

Unless stated to the contrary, the entire disclosure of each of the patent documents and scientific articles referred to herein is incorporated by reference for all purposes. Sequences listed in the accompanying Appendix filed concurrently with this application are incorporated by reference as part of the disclosure described herein for all purposes.

### EQUIVALENTS

The present application may be embodied in other specific forms without departing from the spirit or essential characteristics thereof. The foregoing embodiments are therefore to be considered in all respects illustrative rather than limiting the application described herein. Various structural elements of the different embodiments and various disclosed method steps may be utilized in various combinations and permutations, and all such variants are to be considered forms of the disclosure. Scope of the present application is thus indicated by the appended claims rather than by the foregoing description, and all changes that come within the meaning and range of equivalency of the claims are intended to be embraced therein.

What is claimed is:

1. A protein comprising:
(a) a first antigen-binding site comprising a heavy chain variable domain (VH) and a light chain variable domain (VL) of an anti-NKG2D antibody;
(b) a second antigen-binding site comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 138, 139, and 140, respectively, and the VL comprises CDR1, CDR2, and CDR3 sequences comprising the amino acid sequences of SEQ ID NOs: 141, 142, and 143, respectively; and
(c) an antibody Fc domain or a portion thereof sufficient to bind CD16.

2. A protein comprising:
(a) a first antigen-binding site comprising a heavy chain variable domain (VH) and a light chain variable domain (VL) of an anti-NKG2D antibody;
(b) a second antigen-binding site comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 472, 474, and 140, respectively, and the VL comprises CDR1, CDR2, and CDR3 sequences comprising the amino acid sequences of SEQ ID NOs: 141, 142, and 143, respectively, wherein the CDRs are according to Kabat numbering scheme; and
(c) an antibody Fc domain or a portion thereof sufficient to bind CD16.

3. A protein comprising:
(a) a first antigen-binding site comprising a heavy chain variable domain (VH) and a light chain variable domain (VL) of an anti-NKG2D antibody;
(b) a second antigen-binding site comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 138, 482 and 483, respectively, and the VL comprises CDR1, CDR2, and CDR3 sequences comprising the amino acid sequences of SEQ ID NOs: 484, 485 and 486, respectively, wherein the CDRs are according to Chothia numbering scheme; and
(c) an antibody Fc domain or a portion thereof sufficient to bind CD16.

4. A protein comprising:
(a) a first antigen-binding site comprising a heavy chain variable domain (VH) and a light chain variable domain (VL) of an anti-NKG2D antibody;
(b) a second antigen-binding site comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 499, 500 and 501, respectively, and the VL comprises CDR1, CDR2, and CDR3 sequences comprising the amino acid sequences of SEQ ID NOs: 502, 485 and 143, respectively, wherein the CDRs are according to IMGT numbering scheme; and
(c) an antibody Fc domain or a portion thereof sufficient to bind CD16.

5. A protein comprising:
(a) a first antigen-binding site comprising a heavy chain variable domain (VH) and a light chain variable domain (VL) of an anti-NKG2D antibody;
(b) a second antigen-binding site comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 516, 521 and 518, respectively, and the VL comprises CDR1, CDR2, and CDR3 sequences comprising the amino acid sequences of SEQ ID NOs: 519, 522 and 486, respectively, wherein the CDRs are according to Honegger numbering scheme; and
(c) an antibody Fc domain or a portion thereof sufficient to bind CD16.

6. The protein of any one of claims 1-5, wherein the first antigen-binding site comprising the VH and the VL of the anti-NKG2D antibody is a Fab fragment, and the second antigen-binding site comprising the VH and the VL of the anti-5T4 antibody is an scFv.

7. The protein of any one of claims 1-5, wherein the first antigen-binding site comprising the VH and the VL of the anti-NKG2D antibody is an scFv, and the second antigen-binding site comprising the VH and the VL of the anti-5T4 antibody is a Fab fragment.

8. The protein of any one of claims 1 to 7, further comprising an additional antigen-binding site comprising a VH and a VL of an anti-5T4 antibody.

9. The protein of claim 8, wherein the first antigen-binding site comprising the VH and the VL of the anti-NKG2D antibody is an scFv, and the second and the additional antigen-binding sites comprising the VH and the VL of the anti-5T4 antibody are each a Fab fragment.

10. The protein of claim 8, wherein the first antigen-binding site comprising the VH and the VL of the anti-NKG2D antibody is an scFv, and the second and the additional antigen-binding sites comprising the VH and the VL of the anti-5T4 antibody are each an scFv.

11. The protein of any one of claims 8 to 10, wherein the amino acid sequences of the second and the additional antigen-binding sites are identical.

12. The protein of any one of claims 7 and 10-11, wherein the scFv comprising the VH and the VL of the anti-NKG2D antibody is linked to an antibody constant domain or a portion thereof sufficient to bind CD16 via a hinge comprising Ala-Ser or Gly-Ser

13. The protein of any one of claims 6 and 10-12, wherein each scFv comprising the VH and the VL of the anti-5T4 antibody is linked to an antibody constant domain or a portion thereof sufficient to bind CD16 via a hinge comprising Ala-Ser or Gly-Ser.

14. The protein of any one of claims 6 and 10-13, wherein each scFv comprising the VH and the VL of the anti-5T4 antibody is linked to an antibody constant domain or a portion thereof sufficient to bind CD16 via a hinge comprising Ala-Ser.

15. The protein of claim 12 or claim 13, wherein the hinge further comprises an amino acid sequence Thr-Lys-Gly.

16. The protein of any one of claims 7 and 9-15, wherein within the scFv comprising the VH and the VL of the anti-NKG2D antibody, the VH of the scFv forms a disulfide bridge with the VL of the scFv.

17. The protein of any one of claims 6 and 10-16, wherein within each scFv comprising the VH and the VL of the anti-5T4 antibody, the VH of the scFv forms a disulfide bridge with the VL of the scFv.

18. The protein of claim 16 or claim 17, wherein the disulfide bridge is formed between C44 of the VH and C100 of the VL, numbered under the Kabat numbering scheme.

19. The protein of any one of claims 7 and 9-17, wherein within the scFv comprising the VH and the VL of the anti-NKG2D antibody, the VH is linked to the VL via a flexible linker.

20. The protein of any one of claims 6 and 10-19, wherein within each scFv comprising the VH and the VL of the anti-5T4 antibody, the VH is linked to the VL via a flexible linker.

21. The protein of claim 19 or claim 20, wherein the flexible linker comprises (G₄S)₄ (SEQ ID NO:119).

22. The protein of any one of claims 7 and 9-21, wherein within the scFv comprising the VH and the VL of the anti-NKG2D antibody, the VH is positioned at the C-terminus of the VL.

23. The protein of any one of claims 6 and 10-22, wherein within each scFv comprising the VH and the VL of the anti-5T4 antibody, the VH is positioned at the C-terminus of the VL.

24. The protein of any one of claims 7 and 9-21, wherein within the scFv comprising the VH and the VL of the anti-NKG2D antibody, the VH is positioned at the N-terminus of the VL.

25. The protein of any one of claims 6, 10-22 and 24, wherein within each scFv comprising the VH and the VL of the anti-5T4 antibody, the VH is positioned at the N-terminus of the VL.

26. The protein of any one of claims 6, 13-15, 17-18, 20-21, 23 and 25, wherein the Fab fragment comprising the VH and the VL of the anti-NKG2D antibody is not positioned between an antigen-binding site and the Fc or the portion thereof.

27. The protein of any one of claims 7, 9, 11-12, 15-16, 18-19, 21-22 and 24, wherein no Fab fragment comprising the VH and the VL of the anti-5T4 antibody is positioned between an antigen-binding site and the Fc or the portion thereof.

28. The protein of any one of claims 1-27, wherein the first antigen-binding site binds human NKG2D.

29. The protein of any one of claims 1-28, wherein the second antigen-binding site binds human 5T4.

30. The protein of any one of claims 1-29, wherein the second antigen-binding site binds human 5T4 within an LRR1 domain.

31. The protein of any one of claims 1-30, wherein the CD16 is human CD16.

32. The protein of claim 31, wherein the human CD16 is human CD16a (FcγRIIIa).

33. The protein of any one of claims 1-32, wherein the first antigen-binding site comprising the VH and the VL of the anti-NKG2D antibody comprises a VH comprising CDR1, CDR2, and CDR3 comprising the amino acid sequences of SEQ ID NOs: 81, 82, and 112, respectively; and a VL comprising CDR1, CDR2, and CDR3 comprising the amino acid sequences of SEQ ID NOs: 86, 77, and 87, respectively.

34. A protein comprising:
(a) a first antigen-binding site comprising a VH and a VL of an anti-NKG2D antibody, wherein the VH comprises CDR1, CDR2, and CDR3 sequences represented by the amino acid sequences of SEQ ID NOs: 81, 82, and 112, respectively, and the VL comprises CDR1, CDR2, and CDR3 sequences represented by the amino acid sequences of SEQ ID NOs: 86, 77, and 87, respectively;
(b) a second antigen-binding site comprising a VH and a VL of an anti-5T4 antibody, wherein the VH comprises CDR1, CDR2, and CDR3 sequences represented by the amino acid sequences of SEQ ID NOs: 138, 139, and 140, respectively, and the VL comprises CDR1, CDR2, and CDR3 sequences represented by the amino acid sequences of SEQ ID NOs: 141, 142, and 143, respectively; and
(c) an antibody Fc domain or a portion thereof sufficient to bind CD16.

35. The protein of any one of claims 1-34, wherein the first antigen-binding site comprising the VH and the VL of the anti-NKG2D antibody comprises a VH comprising CDR1, CDR2, and CDR3 sequences represented by the amino acid sequences of SEQ ID NOs: 81, 82, and 97, respectively; and a VL comprising CDR1, CDR2, and CDR3 sequences represented by the amino acid sequences of SEQ ID NOs: 86, 77, and 87, respectively.

36. A protein comprising:
(a) a first antigen-binding site comprising a VH and a VL of an anti-NKG2D antibody, wherein the VH comprises CDR1, CDR2, and CDR3 sequences represented by the amino acid sequences of SEQ ID NOs: 81, 82, and 112, respectively, and the VL comprises CDR1, CDR2, and CDR3 sequences represented by the amino acid sequences of SEQ ID NOs: 86, 77, and 87, respectively;
(b) a second antigen-binding site comprising a VH and a VL of an anti-5T4 antibody, wherein the VH comprises CDR1, CDR2, and CDR3 sequences represented by the amino acid sequences of SEQ ID NOs: 472, 474 and 140, respectively, and the VL comprises CDR1, CDR2, and CDR3 sequences represented by the amino acid sequences of SEQ ID NOs: 141, 142, and 143, respectively, wherein the numbering is according to Kabat numbering scheme; and
(c) an antibody Fc domain or a portion thereof sufficient to bind CD16.

37. The protein of any one of claims 1-33 and 36, wherein the first antigen-binding site comprising the VH and the VL of the anti-NKG2D antibody comprises a VH comprising CDR1, CDR2, and CDR3 sequences represented by the amino acid sequences of SEQ ID NOs: 81, 82, and 97, respectively; and a VL comprising CDR1, CDR2, and CDR3 sequences represented by the amino acid sequences of SEQ ID NOs: 86, 77, and 87, respectively.

38. A protein comprising:
(a) a first antigen-binding site comprising a VH and a VL of an anti-NKG2D antibody, wherein the VH comprises CDR1, CDR2, and CDR3 sequences represented by the amino acid sequences of SEQ ID NOs: 381, 390 and 391, respectively, and the VL comprises CDR1, CDR2, and CDR3 sequences represented by the amino acid sequences of SEQ ID NOs: 392, 385 and 393, respectively;
(b) a second antigen-binding site comprising a VH and a VL of an anti-5T4 antibody, wherein the VH comprises CDR1, CDR2, and CDR3 sequences represented by the amino acid sequences of SEQ ID NOs: 138, 482 and 483, respectively, and the VL comprises CDR1, CDR2, and CDR3 sequences represented by the amino acid sequences of SEQ ID NOs: 484, 485 and 486, respectively, wherein the numbering is according to Chothia numbering scheme; and
(c) an antibody Fc domain or a portion thereof sufficient to bind CD16.

39. The protein of any one of claims 1-33 and 38, wherein the first antigen-binding site comprising the VH and the VL of the anti-NKG2D antibody comprises a VH comprising CDR1, CDR2, and CDR3 sequences represented by the amino acid sequences of SEQ ID NOs: 381, 390 and 395, respectively; and a VL comprising CDR1, CDR2, and CDR3 sequences represented by the amino acid sequences of SEQ ID NOs: 392, 385 and 393, respectively.

40. A protein comprising:
(a) a first antigen-binding site comprising a VH and a VL of an anti-NKG2D antibody, wherein the VH comprises CDR1, CDR2, and CDR3 sequences represented by the amino acid sequences of SEQ ID NOs: 422, 423 and 111, respectively, and the VL comprises CDR1, CDR2, and CDR3 sequences represented by the amino acid sequences of SEQ ID NOs: 424, 385 and 87, respectively;
(b) a second antigen-binding site comprising a VH and a VL of an anti-5T4 antibody, wherein the VH comprises CDR1, CDR2, and CDR3 sequences represented by the amino acid sequences of SEQ ID NOs: 499, 500 and 501, respectively, and the VL comprises CDR1, CDR2, and CDR3 sequences represented by the amino acid sequences of SEQ ID NOs: 502, 485 and 143, respectively, wherein the numbering is according to IMGT numbering scheme; and
(c) an antibody Fc domain or a portion thereof sufficient to bind CD16.

41. The protein of any one of claims 1-33 and 40, wherein the first antigen-binding site comprising the VH and the VL of the anti-NKG2D antibody comprises a VH comprising CDR1, CDR2, and CDR3 sequences represented by the amino acid sequences of SEQ ID NOs: 422, 423 and 96, respectively; and a VL comprising CDR1, CDR2, and CDR3 sequences represented by the amino acid sequences of SEQ ID NOs: 424, 385 and 87, respectively.

42. A protein comprising:
(a) a first antigen-binding site comprising a VH and a VL of an anti-NKG2D antibody, wherein the VH comprises CDR1, CDR2, and CDR3 sequences represented by the amino acid sequences of SEQ ID NOs: 462, 463 and 464, respectively, and the VL comprises CDR1, CDR2, and CDR3 sequences represented by the amino acid sequences of SEQ ID NOs: 465, 459 and 393;
(b) a second antigen-binding site comprising a VH and a VL of an anti-5T4 antibody, wherein the VH comprises CDR1, CDR2, and CDR3 sequences represented by the amino acid sequences of SEQ ID NOs: 516, 521 and 518, respectively, and the VL comprises CDR1, CDR2, and CDR3 sequences represented by the amino acid sequences of SEQ ID NOs: 519, 522 and 486, wherein the numbering is according to Honegger numbering scheme; and
(c) an antibody Fc domain or a portion thereof sufficient to bind CD16.

43. The protein of any one of claims 1-33 and 42, wherein the first antigen-binding site comprising the VH and the VL of the anti-NKG2D antibody comprises a VH comprising CDR1, CDR2, and CDR3 sequences represented by the amino acid sequences of SEQ ID NOs: 462, 463 and 467, respectively; and a VL comprising CDR1, CDR2, and CDR3 sequences represented by the amino acid sequences of SEQ ID NOs: 465, 459 and 393, respectively.

44. The protein of any one of claims 1-43, wherein the antibody Fc domain comprises a first antibody Fc domain polypeptide or a portion thereof sufficient to bind CD16 and a second antibody Fc domain polypeptide or a portion thereof sufficient to bind CD16.

45. The protein of any one of claims 1-44, wherein the VH or the VL of the anti-NKG2D antibody is fused to the N-terminus of the first antibody Fc domain polypeptide or portion thereof sufficient to bind CD16, and the VH or the VL of the anti-5T4 antibody is fused to the N-terminus of the second antibody Fc domain polypeptide or portion thereof sufficient to bind CD16.

46. The protein of any one of claims 1-45, wherein the VH of the anti-NKG2D antibody is fused to the N-terminus of the first antibody Fc domain polypeptide or portion thereof sufficient to bind CD16, and the VH of the anti-5T4 antibody is fused to the N-terminus of the second antibody Fc domain polypeptide or portion thereof sufficient to bind CD16.

47. A protein comprising:
(a) a first antigen-binding site comprising a VH and a VL of an anti-NKG2D antibody, wherein the VH comprises CDR1, CDR2, and CDR3 sequences represented by the amino acid sequences of SEQ ID NOs: 81, 82, and 97, respectively, and the VL comprises CDR1, CDR2, and CDR3 sequences represented by the amino acid sequences of SEQ ID NOs: 86, 77, and 87;
(b) a second antigen-binding site comprising a VH and a VL of an anti-5T4 antibody, wherein the VH comprises CDR1, CDR2, and CDR3 sequences represented by the amino acid sequences of SEQ ID NOs: 138, 139, and 140, respectively, and the VL comprises CDR1, CDR2, and CDR3 sequences represented by the amino acid sequences of SEQ ID NOs: 141, 142, and 143; and
(c) an antibody Fc domain, comprising a first antibody Fc domain polypeptide or a portion thereof sufficient to bind CD16, and a second antibody Fc domain polypeptide or a portion thereof sufficient to bind CD16; and
wherein the VH or the VL of the anti-NKG2D antibody is fused to the N-terminus of the first antibody Fc domain polypeptide or portion thereof sufficient to bind CD16, and the VH or the VL of the anti-5T4 antibody is fused to the N-terminus of the second antibody Fc domain polypeptide or portion thereof sufficient to bind CD16.

48. A protein comprising:
(a) a first antigen-binding site comprising a VH and a VL of an anti-NKG2D antibody, wherein the VH comprises CDR1, CDR2, and CDR3 sequences represented by the amino acid sequences of SEQ ID NOs: 81, 82, and 97, respectively, and the VL comprises CDR1, CDR2, and CDR3 sequences represented by the amino acid sequences of SEQ ID NOs: 86, 77, and 87;
(b) a second antigen-binding site comprising a VH and a VL of an anti-5T4 antibody, wherein the VH comprises CDR1, CDR2, and CDR3 sequences represented by the amino acid sequences of SEQ ID NOs: 138, 139, and 140, respectively, and the VL comprises CDR1, CDR2, and CDR3 sequences represented by the amino acid sequences of SEQ ID NOs: 141, 142, and 143; and
(c) an antibody Fc domain, comprising a first antibody Fc domain polypeptide or a portion thereof, and a second antibody Fc domain polypeptide or a portion thereof, wherein the first antibody Fc domain polypeptide or portion thereof and the second antibody Fc domain polypeptide or portion thereof together are capable of binding CD16; and
wherein the VH or the VL of the anti-NKG2D antibody is fused to the N-terminus of the first antibody Fc domain polypeptide or portion thereof sufficient to bind CD16, and the VH or the VL of the anti-5T4 antibody is fused to the N-terminus of the second antibody Fc domain polypeptide or portion thereof sufficient to bind CD16.

49. The protein of any one of claims 1-48, wherein the first antigen-binding site comprises a VH comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:95 and a VL comprising an amino acid sequence at least 90%, e.g., at least 91%, at least 92%, at least 93%, at least 94%, at least 95%, at least 96%, at least 97%, at least 98%, at least 99% or 100%, identical to SEQ ID NO:85.

50. The protein of any one of claims 1-49, wherein the first antigen-binding site comprises a VH comprising an amino acid sequence at least 95% identical to SEQ ID NO:95 and a VL comprising an amino acid sequence at least 95% identical to SEQ ID NO:85.

51. The protein of any one of claims 1-50, wherein the first antigen-binding site comprises a VH comprising an amino acid sequence at least 96% identical to SEQ ID NO:95 and a VL comprising an amino acid sequence at least 96% identical to SEQ ID NO:85.

52. The protein of any one of claims 1-51, wherein the first antigen-binding site comprises a VH comprising an amino acid sequence at least 97% identical to SEQ ID NO:95 and a VL comprising an amino acid sequence at least 97% identical to SEQ ID NO:85.

53. The protein of any one of claims 1-52, wherein the first antigen-binding site comprises a VH comprising an amino acid sequence at least 98% identical to SEQ ID NO:95 and a VL comprising an amino acid sequence at least 98% identical to SEQ ID NO:85.

54. The protein of any one of claims 1-53, wherein the first antigen-binding site comprises a VH comprising an amino acid sequence at least 99% identical to SEQ ID NO:95 and a VL comprising an amino acid sequence at least 99% identical to SEQ ID NO:85.

55. The protein of any one of claims 1-54, wherein the first antigen-binding site comprises a VH comprising an amino acid sequence of SEQ ID NO:95 and a VL comprising an amino acid sequence of SEQ ID NO:85.

56. The protein of any one of claims 1-55, wherein the second antigen-binding site comprises a VH at least 90%, e.g., at least 91%, at least 92%, at least 93%, at least 94%, at least 95%, at least 96%, at least 97%, at least 98%, at least 99% or 100%, identical to SEQ ID NO:144 and a VL at least 90%, e.g., at least 91%, at least 92%, at least 93%, at least 94%, at least 95%, at least 96%, at least 97%, at least 98%, at least 99% or 100%, identical to SEQ ID NO:145.

57. The protein of any one of claims 1-55, wherein the second antigen-binding site comprises a VH comprising the amino acid sequence at least 90%, e.g., at least 91%, at least 92%, at least 93%, at least 94%, at least 95%, at least 96%, at least 97%, at least 98%, at least 99% or 100%, identical to SEQ ID NO:263 and a VL comprising the amino acid sequence at least 90%, e.g., at least 91%, at least 92%, at least 93%, at least 94%, at least 95%, at least 96%, at least 97%, at least 98%, at least 99% or 100%, identical to SEQ ID NO:145.

58. The protein of claim 56 or claim 57, wherein the second antigen-binding site comprises a VH with a G44C substitution relative to SEQ ID NO:144 and a VL with a G100C substitution relative to SEQ ID NO:145, numbered under the Kabat numbering scheme.

59. The protein of any one of claims 1-57, wherein the second antigen-binding site comprises a VH comprising the amino acid sequence of SEQ ID NO:144 and a VL comprising the amino acid sequence of SEQ ID NO:145, or a VH comprising the amino acid sequence at least 90%, e.g., at least 91%, at least 92%, at least 93%, at least 94%, at least 95%, at least 96%, at least 97%, at least 98%, at least 99% or 100%, identical to SEQ ID NO:146 and a VL comprising the amino acid sequence at least 90%, e.g., at least 91%, at least 92%, at least 93%, at least 94%, at least 95%, at least 96%, at least 97%, at least 98%, at least 99% or 100%, identical to SEQ ID NO:147.

60. The protein of any one of claims 1-55 and 59, wherein the second antigen-binding site comprises a VH comprising the amino acid sequence at least 90%, e.g., at least 91%, at least 92%, at least 93%, at least 94%, at least 95%, at least 96%, at least 97%, at least 98%, at least 99% or 100%, identical to SEQ ID NO:146 and a VL comprising the amino acid sequence at least 90%, e.g., at least 91%, at least 92%, at least 93%, at least 94%, at least 95%, at least 96%, at least 97%, at least 98%, at least 99% or 100%, identical to SEQ ID NO:147.

61. The protein of any one of claims 1-55 and 59-60, wherein the second antigen-binding site comprises a VH comprising the amino acid sequence at least 95% identical to SEQ ID NO:146 and a VL comprising the amino acid sequence at least 95% identical to SEQ ID NO: 147.

62. The protein of any one of claims 1-55 and 59-61, wherein the second antigen-binding site comprises a VH comprising the amino acid sequence at least 96% identical to SEQ ID NO:146 and a VL comprising the amino acid sequence at least 96% identical to SEQ ID NO: 147.

63. The protein of any one of claims 1-55 and 59-62, wherein the second antigen-binding site comprises a VH comprising the amino acid sequence at least 97% identical to SEQ ID NO:146 and a VL comprising the amino acid sequence at least 97% identical to SEQ ID NO: 147.

64. The protein of any one of claims 1-55 and 59-63, wherein the second antigen-binding site comprises a VH comprising the amino acid sequence at least 98% identical to SEQ ID NO:146 and a VL comprising the amino acid sequence at least 98% identical to SEQ ID NO: 147.

65. The protein of any one of claims 1-55 and 59-64, wherein the second antigen-binding site comprises a VH comprising the amino acid sequence at least 99% identical to SEQ ID NO:146 and a VL comprising the amino acid sequence at least 99% identical to SEQ ID NO: 147.

66. The protein of any one of claims 1-55 and 59-65, wherein the second antigen-binding site comprises a VH comprising the amino acid sequence of SEQ ID NO:146 and a VL comprising the amino acid sequence of SEQ ID NO:147.

67. The protein of any one of claims 1-59, wherein the second antigen-binding site comprises a VH comprising the amino acid sequence of SEQ ID NO:144 and a VL comprising the amino acid sequence of SEQ ID NO:145.

68. The protein of any one of claims 1, 6, 13-15, 17-18, 20-21, 23, 25-26, 28-55 and 59-66, wherein the second antigen-binding site comprises a single-chain fragment variable (scFv), and wherein the scFv comprises a VH comprising the amino acid sequence of SEQ ID NO:146 and a VL comprising the amino acid sequence of SEQ ID NO:147.

69. The protein of any one of claims 1, 6, 13-15, 17-18, 20-21, 23, 25-26, 28-55 and 59-66, wherein the second antigen-binding site comprises a single-chain fragment variable (scFv), and wherein the scFv comprises an amino acid sequence at least 90% identical, e.g., at least 91%, 92%, 93%, 94%, 95%, 96%, 97%, 98% or 99% identical, to a sequence selected from the group consisting of SEQ ID NOs: 148 and 149.

70. The protein of any one of claims 1, 6, 13-15, 17-18, 20-21, 23, 25-26, 28-55 and 59-66, wherein the second antigen-binding site comprises an scFv and the scFv comprises an amino acid sequence at least 90% identical, e.g., at least 91%, 92%, 93%, 94%, 95%, 96%, 97%, 98% or 99% identical, to SEQ ID NO:148.

71. The protein of any one of claims 1, 6, 13-15, 17-18, 20-21, 23, 25-26, 28-55, 59-66 and 70, wherein the second antigen-binding site comprises an scFv and the scFv comprises an amino acid sequence at least 95% identical to SEQ ID NO:148.

72. The protein of any one of claims 1, 6, 13-15, 17-18, 20-21, 23, 25-26, 28-55, 59-66 and 70-71, wherein the second antigen-binding site comprises an scFv and the scFv comprises an amino acid sequence at least 96% identical to SEQ ID NO:148.

73. The protein of any one of claims 1, 6, 13-15, 17-18, 20-21, 23, 25-26, 28-55, 59-66 and 70-72, wherein the second antigen-binding site comprises an scFv and the scFv comprises an amino acid sequence at least 97% identical to SEQ ID NO:148.

74. The protein of any one of claims 1, 6, 13-15, 17-18, 20-21, 23, 25-26, 28-55, 59-66 and 70-73, wherein the second antigen-binding site comprises an scFv and the scFv comprises an amino acid sequence at least 98% identical to SEQ ID NO:148.

75. The protein of any one of claims 1, 6, 13-15, 17-18, 20-21, 23, 25-26, 28-55, 59-66 and 70-74, wherein the second antigen-binding site comprises an scFv and the scFv comprises an amino acid sequence at least 99% identical to SEQ ID NO:148.

76. The protein of any one of claims 1, 6, 13-15, 17-18, 20-21, 23, 25-26, 28-55, 59-66 and 70-75, wherein the second antigen-binding site comprises an scFv and the scFv comprising an amino acid sequence of SEQ ID NO:148.

77. The protein of any one of claims 1, 6, 13-15, 17-18, 20-21, 23, 25-26, 28-55, 59-66 and 70-76, wherein the protein comprises an amino acid sequence at least 90% identical, e.g., at least 91%, 92%, 93%, 94%, 95%, 96%, 97%, 98% or 99% identical, to SEQ ID NO:198.

78. The protein of any one of claims 1, 6, 13-15, 17-18, 20-21, 23, 25-26, 28-55, 59-66 and 70-77, wherein the protein comprises an amino acid sequence at least 95% identical to SEQ ID NO:198.

79. The protein of any one of claims 1, 6, 13-15, 17-18, 20-21, 23, 25-26, 28-55, 59-66 and 70-78, wherein the protein comprises an amino acid sequence at least 96% identical to SEQ ID NO:198.

80. The protein of any one of claims 1, 6, 13-15, 17-18, 20-21, 23, 25-26, 28-55, 59-66 and 70-79, wherein the protein comprises an amino acid sequence at least 97% identical to SEQ ID NO:198.

81. The protein of any one of claims 1, 6, 13-15, 17-18, 20-21, 23, 25-26, 28-55, 59-66 and 70-80, wherein the protein comprises an amino acid sequence at least 98% identical to SEQ ID NO:198.

82. The protein of any one of claims 1, 6, 13-15, 17-18, 20-21, 23, 25-26, 28-55, 59-66 and 70-81, wherein the protein comprises an amino acid sequence at least 99% identical to SEQ ID NO:198.

83. The protein of any one of claims 1, 6, 13-15, 17-18, 20-21, 23, 25-26, 28-55, 59-66 and 70-82, wherein the protein comprises an amino acid sequence of SEQ ID NO:198.

84. A protein comprising:
(a) a first antigen-binding site comprising a VH and a VL of an anti-NKG2D antibody, wherein the VH comprises the amino acid sequence of SEQ ID NO:95 and the VL comprises the amino acid sequence of SEQ ID NO:85;
(b) a second antigen-binding site comprising a VH and a VL of an anti-5T4 antibody, wherein the VH comprises the amino acid sequence of SEQ ID NO:146 and the VL comprises the amino acid sequence of SEQ ID NO:147; and
(c) an antibody Fc domain, comprising a first antibody Fc domain polypeptide or a portion thereof sufficient to bind CD16, and a second antibody Fc domain polypeptide or a portion thereof sufficient to bind CD16.

85. A protein comprising:
(a) a first antigen-binding site comprising a VH and a VL of an anti-NKG2D antibody, wherein the VH comprises the amino acid sequence of SEQ ID NO:95 and the VL comprises the amino acid sequence of SEQ ID NO:85;
(b) a second antigen-binding site comprising the amino acid sequence of SEQ ID NO:148; and
(c) an antibody Fc domain, comprising a first antibody Fc domain polypeptide or a portion thereof sufficient to bind CD16, and a second antibody Fc domain polypeptide or a portion thereof sufficient to bind CD16.

86. A protein comprising:
(a) a first antigen-binding site comprising a heavy chain variable domain (VH) and a light chain variable domain (VL) of an anti-NKG2D antibody;
(b) a second antigen-binding site comprising a VH comprising an CDR1, a CDR2, and a CDR3 sequence selected from Table 2 and a VL comprising a CDR1, a CDR2, and a CDR3 sequence selected from Table 2, or a VH comprising a CDR1, a CDR2, and a CDR3 sequence selected from Table 12 and a VL comprising a CDR1, a CDR2, and a CDR3 comprising the amino acid sequences of SEQ ID NOs: 189, 190, and 143, respectively; and
(c) an antibody Fc domain or a portion thereof sufficient to bind CD16.

87. The protein of any one of claims 1-86, wherein the antibody Fc domain is a human IgG1 antibody Fc domain.

88. The protein of claim 87, wherein the antibody Fc domain or the portion thereof comprises an amino acid sequence at least 90%, at least 91%, at least 92%, at least 93%, at least 94%, at least 95%, at least 96%, at least 97%, at least 98%, at least 99% or 100%, identical to SEQ ID NO:118.

89. The protein of any one of claims 87-88, wherein the antibody Fc domain or the portion thereof comprises an amino acid sequence at least 95% identical to SEQ ID NO:118.

90. The protein of any one of claims 87-89, wherein the antibody Fc domain or the portion thereof comprises an amino acid sequence at least 96% identical to SEQ ID NO:118.

91. The protein of any one of claims 87-90, wherein the antibody Fc domain or the portion thereof comprises an amino acid sequence at least 97% identical to SEQ ID NO:118.

92. The protein of any one of claims 87-91, wherein the antibody Fc domain or the portion thereof comprises an amino acid sequence at least 98% identical to SEQ ID NO:118.

93. The protein of any one of claims 87-92, wherein at least one polypeptide chain of the antibody Fc domain comprises one or more mutations, relative to SEQ ID NO:118, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system.

94. The protein of any one of claims 87-93, wherein at least one polypeptide chain of the antibody Fc domain comprises one or more mutations, relative to SEQ ID NO:118, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, F405L, Y407A, Y407I, Y407V, K409F, K409W, K409D, K409R, T411D, T411E, K439D, and K439E, numbered according to the EU numbering system.

95. The protein of any one of claims 87-94, wherein one polypeptide chain of the antibody heavy chain constant region comprises one or more mutations, relative to SEQ ID NO:118, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:118, at one or more positions selected from Q347, Y349, L351, S354, E356, E357, S364, T366, L368, K370, N390, K392, T394, D399, D401, F405, Y407, K409, T411, and K439, numbered according to the EU numbering system.

96. The protein of claim 95, wherein one polypeptide chain of the antibody heavy chain constant region comprises K360E and K409W substitutions relative to SEQ ID NO:118; and the other polypeptide chain of the antibody heavy chain constant region comprises Q347R, D399V and F405T substitutions relative to SEQ ID NO:118, numbered according to the EU numbering system.

97. The protein of claim 96, wherein the VH of the anti-NKG2D antibody is fused to the N-terminus of a first antibody Fc domain polypeptide comprising K360E and K409W substitutions relative to SEQ ID NO:118, and the VH of the anti-5T4 antibody is fused to the N-terminus of a second antibody Fc domain polypeptide comprising Q347R, D399V and F405T substitutions relative to SEQ ID NO:118, numbered according to the EU numbering system.

98. The protein of any one of claims 96-97, wherein the first antibody Fc domain polypeptide and the second antibody Fc domain polypeptide form a heterodimer.

99. The protein of any one of claims 96-98, wherein heterodimer formation is facilitated by the K360E and K409W substitutions in the first antibody Fc domain polypeptide and the Q347R, D399V and F405T substitutions in the second antibody Fc domain polypeptide.

100. The protein of claim 95, wherein one polypeptide chain of the antibody heavy chain constant region comprises an F405L substitution relative to SEQ ID NO:118; and the other polypeptide chain of the antibody heavy chain constant region comprises a K409R substitution relative to SEQ ID NO:118, numbered according to the EU numbering system.

101. The protein of any one of claims 95-100, wherein one polypeptide chain of the antibody heavy chain constant region comprises a Y349C substitution relative to SEQ ID NO:118; and the other polypeptide chain of the antibody heavy chain constant region comprises an S354C substitution relative to SEQ ID NO:118, numbered according to the EU numbering system.

102. The protein of claim 101, wherein the VH of the anti-NKG2D antibody is fused to the N-terminus of a first antibody Fc domain polypeptide comprising a Y349C substitution relative to SEQ ID NO:118, and the VH of the anti-5T4 antibody is fused to the N-terminus of a second antibody Fc domain polypeptide comprising a S354C substitution relative to SEQ ID NO:118, numbered according to the EU numbering system.

103. The protein of any one of claims 101-102, wherein the first antibody Fc domain polypeptide forms a disulfide bridge with the second antibody Fc domain polypeptide.

104. The protein of any one of claims 101-103, wherein the disulfide bridge is formed between the Y349C substitution in the first antibody Fc domain polypeptide and the S354C substitution in the second antibody Fc domain polypeptide, numbered according to the EU numbering system.

105. The protein of any one of claims 1, 6, 13-15, 17-18, 20-21, 23, 25-26, 28-55, 59-66 and 70-104, wherein the protein comprises:
(a) a first polypeptide comprising an amino acid sequence at least 90%, at least 91%, at least 92%, at least 93%, at least 94%, at least 95%, at least 96%, at least 97%, at least 98%, at least 99% or 100%, identical to SEQ ID NO:198;
(b) a second polypeptide comprising an amino acid sequence at least 90%, at least 91%, at least 92%, at least 93%, at least 94%, at least 95%, at least 96%, at least 97%, at least 98%, at least 99% or 100%, identical to SEQ ID NO:199; and
(c) a third polypeptide comprising an amino acid sequence at least 90%, at least 91%, at least 92%, at least 93%, at least 94%, at least 95%, at least 96%, at least 97%, at least 98%, at least 99% or 100%, identical to SEQ ID NO:200.

106. The protein of any one of claims 1, 6, 13-15, 17-18, 20-21, 23, 25-26, 28-55, 59-66 and 70-105, wherein the protein comprises:
(a) a first polypeptide comprising an amino acid sequence at least 95% identical to SEQ ID NO:198;
(b) a second polypeptide comprising an amino acid sequence at least 95% identical to SEQ ID NO:199; and
(c) a third polypeptide comprising an amino acid sequence at least 95% identical to SEQ ID NO:200.

107. The protein of any one of claims 1, 6, 13-15, 17-18, 20-21, 23, 25-26, 28-55, 59-66 and 70-106, wherein the protein comprises:
(a) a first polypeptide comprising the amino acid sequence of SEQ ID NO:198;
(b) a second polypeptide comprising the amino acid sequence of SEQ ID NO:199; and
(c) a third polypeptide comprising the amino acid sequence of SEQ ID NO:200.

108. A protein comprising:
(a) a first polypeptide comprising an amino acid sequence at least 90%, at least 91%, at least 92%, at least 93%, at least 94%, at least 95%, at least 96%, at least 97%, at least 98%, at least 99% or 100%, identical to SEQ ID NO:198;
(b) a second polypeptide comprising an amino acid sequence at least 90%, at least 91%, at least 92%, at least 93%, at least 94%, at least 95%, at least 96%, at least 97%, at least 98%, at least 99% or 100%, identical to SEQ ID NO:199; and
(c) a third polypeptide comprising an amino acid sequence at least 90%, at least 91%, at least 92%, at least 93%, at least 94%, at least 95%, at least 96%, at least 97%, at least 98%, at least 99% or 100%, identical to SEQ ID NO:200.

109. The protein of any one of claims 105-108, wherein:
i) a human NKG2D-binding site is formed by a VH in SEQ ID NO:199 (SEQ ID NO:95) and a VL in SEQ ID NO:200 (SEQ ID NO:85),
ii) a human 5T4-binding site is formed by a VH in SEQ ID NO:198 (SEQ ID NO:146) and a VL in SEQ ID NO:198 (SEQ ID NO:147), and
iii) a human CD16a-binding site is formed by an Fc binding domain polypeptide in SEQ ID NO:198 and an Fc binding domain polypeptide in SEQ ID NO:199.

110. The protein of any one of claims 105-109, wherein:
i) a disulfide bridge is formed between C44 in SEQ ID NO:146 and C100 in SEQ ID NO:147, numbered under the Kabat numbering scheme,
ii) a disulfide bridge is formed between C349 in SEQ ID NO:199 and C354 in SEQ ID NO:198, numbered according to the EU numbering system, and
iii) a heterodimer is formed between an Fc domain in SEQ ID NO:198 and an Fc domain in SEQ ID NO:199.

111. The protein of any one of claims 105-110, wherein the protein is a trispecific antibody.

112. The protein of any one of claims 105-111, wherein the trispecific antibody is capable of binding to human NKG2D and human CD16a on the surface of an NK cell and to human 5T4 on the surface of a tumor cell.

113. A trispecific antibody comprising:
(a) a human NKG2D-binding site which is a Fab fragment comprising a VH and VL, wherein the VH comprises CDR1, CDR2, and CDR3 sequences represented by the amino acid sequences of SEQ ID NOs: 81, 82, and 97, respectively, and the VL comprises CDR1, CDR2, and CDR3 sequences represented by the amino acid sequences of SEQ ID NOs: 86, 77, and 87, respectively,
(b) a human 5T4-binding site which is an scFv comprising a VH and a VL, wherein the VH comprises CDR1, CDR2, and CDR3 sequences comprising the amino acid sequences of SEQ ID NOs: 138, 139, and 140, respectively, and the VL comprises CDR1, CDR2, and CDR3 sequences comprising the amino acid sequences of SEQ ID NOs: 141, 142, and 143, respectively,
wherein the VH is positioned at the C-terminus of the VL,
wherein the VH is linked to the VL via a flexible linker comprising (G₄S)₄ (SEQ ID NO:119), and
wherein a disulfide bridge is formed between C44 of the VH and C100 of the VL, numbered under the Kabat numbering scheme, and
(c) a human CD16a-binding site which is a human IgG1 antibody Fc domain comprising:
(i) a first Fc domain polypeptide that comprises an amino acid sequence at least 95% identical to SEQ ID NO:118 and comprising Y349C, K360E, and K409W substitutions relative to SEQ ID NO:118, numbered according to the EU numbering system, and
(ii) a second Fc domain polypeptide that comprises an amino acid sequence at least 95% identical to SEQ ID NO:118 and comprising Q347R, S354C, D399V and F405T substitutions relative to SEQ ID NO:118, numbered according to the EU numbering system,
wherein a disulfide bridge is formed between C349 of (i) and C354 of (ii), and
wherein (i) and (iii) form a heterodimer,
wherein the VH of (a) is fused to the N-terminus of the first Fc domain polypeptide, and the VH of (b) is fused to the N-terminus of the second Fc domain polypeptide via a hinge comprising Ala-Ser.

114. The trispecific antibody of claim 113, wherein:
the VH of (a) comprises the amino acid sequence of SEQ ID NO:95,
the VL of (a) comprises the amino acid sequence of SEQ ID NO:85,
the VH of (b) comprises the amino acid sequence of SEQ ID NO:146,
the VL of (b) comprises the amino acid sequence of SEQ ID NO:147,
the first Fc domain polypeptide comprises an amino acid sequence at least 98% identical to SEQ ID NO:118, and
the second Fc domain polypeptide comprises an amino acid sequence at least 98% identical to SEQ ID NO:118.

115. The trispecific antibody of claim 114, wherein (b) comprises the amino acid sequence of SEQ ID NO:148.

116. The trispecific antibody of any one of claims 113-115, wherein the trispecific antibody comprises:
(a) a first polypeptide comprising the amino acid sequence of SEQ ID NO:198;
(b) a second polypeptide comprising the amino acid sequence of SEQ ID NO:199; and
(c) a third polypeptide comprising the amino acid sequence of SEQ ID NO:200.

117. A pharmaceutical composition comprising a protein or an antibody according to any one of claims 1-116 and a pharmaceutically acceptable carrier.

118. A cell comprising one or more nucleic acids encoding a protein or antibody according to any one of claims 1-116.

119. A pharmaceutical formulation comprising:
a) a protein comprising:
i) a first antigen-binding site comprising a heavy chain variable domain (VH) and a light chain variable domain (VL) of an anti-NKG2D antibody;
ii) a second antigen-binding site comprising a VH and a VL of an anti-5T4 antibody; and
iii) an antibody Fc domain or a portion thereof sufficient to bind CD16;
b) a buffer comprising citrate;
c) sucrose; and
d) a polysorbate,
wherein the pH of the pharmaceutical formulation is 6.0 to 7.0.

120. The pharmaceutical formulation of claim 119, wherein the concentration of the protein in the pharmaceutical formulation is 1 mg/mL to 125 mg/mL.

121. The pharmaceutical formulation of claim 119 or claim 120, wherein the concentration of the protein in the pharmaceutical formulation is 2 mg/mL to 100 mg/mL.

122. The pharmaceutical formulation of any one of claims 119-121, wherein the concentration of the protein in the pharmaceutical formulation is 5 mg/mL to 50 mg/mL.

123. The pharmaceutical formulation of any one of claims 119-122, wherein the concentration of the protein in the pharmaceutical formulation is 7.5 mg/mL to 25 mg/mL.

124. The pharmaceutical formulation of any one of claims 119-123, wherein the concentration of the protein in the pharmaceutical formulation is 10 mg/mL to 20 mg/mL.

125. The pharmaceutical formulation of any one of claims 119-124, wherein the concentration of the protein in the pharmaceutical formulation is about 15 mg/mL.

126. The pharmaceutical formulation of any one of claims 119-125, wherein the concentration of citrate in the pharmaceutical formulation is 15 mM to 25 mM.

127. The pharmaceutical formulation of any one of claims 119-126, wherein the concentration of citrate in the pharmaceutical formulation is 17.5 mM to 22.5 mM.

128. The pharmaceutical formulation of any one of claims 119-127, wherein the concentration of citrate in the pharmaceutical formulation is about 20 mM.

129. The pharmaceutical formulation of any one of claims 119-128, wherein the buffer comprising citrate in the pharmaceutical formulation comprises sodium citrate, citric acid, or a combination thereof.

130. The pharmaceutical formulation of any one of claims 119-129, wherein the buffer in the pharmaceutical formulation comprises a combination of sodium citrate and citric acid.

131. The pharmaceutical formulation of claims 129-130, wherein the concentration of sodium citrate in the pharmaceutical formulation is 17 mM to 21 mM.

132. The pharmaceutical formulation of any one of claims 129-131, wherein the concentration of sodium citrate in the pharmaceutical formulation is about 18.9 mM.

133. The pharmaceutical formulation of any one of claims 129-132, wherein the concentration of citric acid in the pharmaceutical formulation is 0.5 mM to 1.5 mM.

134. The pharmaceutical formulation of any one of claims 129-133, wherein the concentration of citric acid in the pharmaceutical formulation is about 1.1 mM.

135. The pharmaceutical formulation of any one of claims 119-134, wherein the pH of the buffer in the pharmaceutical formulation is 6.0 to 7.0.

136. The pharmaceutical formulation of any one of claims 119-135, wherein the pH of the buffer in the pharmaceutical formulation is 6.5.

137. The pharmaceutical formulation of any one of claims 119-136, wherein the concentration of sucrose in the pharmaceutical formulation is 170 mM to 180 mM.

138. The pharmaceutical formulation of any one of claims 119-137, wherein the concentration of sucrose in the pharmaceutical formulation is 172.5 mM to 177.5 mM.

139. The pharmaceutical formulation of any one of claims 119-138, wherein the concentration of sucrose in the pharmaceutical formulation is about 175.2 mM.

140. The pharmaceutical formulation of any one of claims 119-139, wherein the polysorbate in the pharmaceutical formulation is polysorbate 80.

141. The pharmaceutical formulation of any one of claims 119-140, wherein the concentration of the polysorbate in the pharmaceutical formulation is 0.05 mg/mL to 0.15 mg/mL.

142. The pharmaceutical formulation of any one of claims 119-141, wherein the concentration of the polysorbate in the pharmaceutical formulation is about 0.1 mg/mL.

143. The pharmaceutical formulation of any one of claims 119-142, wherein the pH of the pharmaceutical formulation is 6.5.

144. A vial comprising a pharmaceutical formulation comprising:
a) a protein comprising:
i) a first antigen-binding site comprising a heavy chain variable domain (VH) and a light chain variable domain (VL) of an anti-NKG2D antibody;
ii) a second antigen-binding site comprising a VH and a VL of an anti-5T4 antibody; and
iii) an antibody Fc domain or a portion thereof sufficient to bind CD16;
b) a buffer comprising citrate;
c) sucrose; and
d) a polysorbate,
wherein the pH of the pharmaceutical formulation is 6.0 to 7.0.

145. The vial of claim 144, wherein the vial comprises 100 mg to 200 mg of the protein.

146. The vial of claim 144 or claim 145, wherein the vial comprises about 150 mg of the protein.

147. The vial of any one of claims 144-146, wherein the vial comprises 50 mg to 60 mg of sodium citrate.

148. The vial of any one of claims 144-147, wherein the vial comprises about 55.5 mg of sodium citrate.

149. The vial of any one of claims 144-148, wherein the vial comprises 1.5 mg to 3 mg of citric acid.

150. The vial of any one of claims 144-149, wherein the vial comprises about 2.3 mg of citric acid.

151. The vial of any one of claims 144-150, wherein the vial comprises 500 mg to 700 mg of sucrose.

152. The vial of any one of claims 144-151, wherein the vial comprises about 600 mg of sucrose.

153. The vial of any one of claims 144-152, wherein the polysorbate in the pharmaceutical formulation is polysorbate 80.

154. The vial of claim 153, wherein the vial comprises 0.5 mg to 1.5 mg of polysorbate 80.

155. The vial of claim 153 or claim 154, wherein the vial comprises about 1 mg of polysorbate 80.

156. The vial of any one of claims 144-155, wherein the pH of the pharmaceutical formulation is 6.5.

157. The vial of any one of claims 144-156, wherein the vial comprises about 10 mL of the pharmaceutical formulation.

158. The pharmaceutical formulation of any one of claims 119-143, or the vial of any one of claims 144-157, wherein more than 93% of the protein has native conformation as determined by size-exclusion chromatography, after incubation at 50° C. for 28 days.

159. The pharmaceutical formulation of any one of claims 119-143 and 158, or the vial of any one of claims 144-158, wherein the protein comprises:
(a) a first antigen-binding site comprising a heavy chain variable domain (VH) and a light chain variable domain (VL) of an anti-NKG2D antibody;
(b) a second antigen-binding site comprising a VH and a VL of an anti-5T4 antibody, wherein the VH comprises complementarity-determining region 1 (CDR1), complementarity-determining region 2 (CDR2), and complementarity-determining region 3 (CDR3) sequences comprising the amino acid sequences of SEQ ID NOs: 138, 139, and 140, respectively, and the VL comprises CDR1, CDR2, and CDR3 sequences comprising the amino acid sequences of SEQ ID NOs: 141, 142, and 143, respectively; and
(c) an antibody Fc domain or a portion thereof sufficient to bind CD16.

160. The pharmaceutical formulation of any one of claims 119-143 and 158-159, or the vial of any one of claims 144-159, wherein the first antigen-binding site comprising the VH and the VL of the anti-NKG2D antibody is a Fab fragment, and the second antigen-binding site comprising the VH and the VL of the anti-5T4 antibody is an scFv.

161. The pharmaceutical formulation of any one of claims 119-143 and 158-159, or the vial of any one of claims 144-159, wherein the first antigen-binding site comprising the VH and the VL of the anti-NKG2D antibody is an scFv, and the second antigen-binding site comprising the VH and the VL of the anti-5T4 antibody is a Fab fragment.

162. The pharmaceutical formulation of any one of claims 119-143 and 158-159, or the vial of any one of claims 144-159, wherein the protein further comprises an additional antigen-binding site comprising a VH and a VL of an anti-5T4 antibody.

163. The pharmaceutical formulation or the vial of claim 162, wherein the first antigen-binding site comprising the VH and the VL of the anti-NKG2D antibody is an scFv, and the second and the additional antigen-binding sites comprising the VH and the VL of the anti-5T4 antibody are each a Fab fragment.

164. The pharmaceutical formulation or the vial of claim 162, wherein the first antigen-binding site comprising the VH and the VL of the anti-NKG2D antibody is an scFv, and the second and the additional antigen-binding sites comprising the VH and the VL of the anti-5T4 antibody are each an scFv.

165. The pharmaceutical formulation or the vial of any one of claims 162-164, wherein the amino acid sequences of the second and the additional antigen-binding sites are identical.

166. The pharmaceutical formulation or the vial of any one of claims 161 and 164-165, wherein the scFv comprising the VH and the VL of the anti-NKG2D antibody is linked to an antibody constant domain or a portion thereof sufficient to bind CD16 via a hinge comprising Ala-Ser or Gly-Ser.

167. The pharmaceutical formulation or the vial of any one of claims 160 and 164-166, wherein each scFv comprising the VH and the VL of the anti-5T4 antibody is linked to an antibody constant domain or a portion thereof sufficient to bind CD16 via a hinge comprising Ala-Ser or Gly-Ser.

168. The pharmaceutical formulation or the vial of any one of claims 160 and 164-167, wherein each scFv comprising the VH and the VL of the anti-5T4 antibody is linked to an antibody constant domain or a portion thereof sufficient to bind CD16 via a hinge comprising Ala-Ser.

169. The pharmaceutical formulation or the vial of any one of claims 166-168, wherein the hinge further comprises an amino acid sequence Thr-Lys-Gly.

170. The pharmaceutical formulation or the vial of any one of claims 161 and 163-169, wherein within the scFv comprising the VH and the VL of the anti-NKG2D antibody, the VH of the scFv forms a disulfide bridge with the VL of the scFv.

171. The pharmaceutical formulation or the vial of any one of claims 160 and 164-170, wherein within each scFv comprising the VH and the VL of the anti-5T4 antibody, the VH of the scFv forms a disulfide bridge with the VL of the scFv.

172. The pharmaceutical formulation or the vial of claim 170 or claim 171, wherein the disulfide bridge is formed between C44 of the VH and C100 of the VL, numbered under the Kabat numbering scheme.

173. The pharmaceutical formulation or the vial of any one of claims 161 and 163-172, wherein within the scFv comprising the VH and the VL of the anti-NKG2D antibody, the VH is linked to the VL via a flexible linker.

174. The pharmaceutical formulation or the vial of any one of claims 160 and 164-173, wherein within each scFv comprising the VH and the VL of the anti-5T4 antibody, the VH is linked to the VL via a flexible linker.

175. The pharmaceutical formulation or the vial of claim 173-174, wherein the flexible linker comprises (G₄S)₄ (SEQ ID NO:119).

176. The pharmaceutical formulation or the vial of any one of claims 161 and 163-175, wherein within the scFv comprising the VH and the VL of the anti-NKG2D antibody, the VH is positioned at the C-terminus of the VL.

177. The pharmaceutical formulation or the vial of any one of claims 159 and 164-176, wherein within each scFv comprising the VH and the VL of the anti-5T4 antibody, the VH is positioned at the C-terminus of the VL.

178. The pharmaceutical formulation or the vial of any one of claims 161 and 163-175 and 177, wherein within the scFv comprising the VH and the VL of the anti-NKG2D antibody, the VH is positioned at the N-terminus of the VL.

179. The pharmaceutical formulation or the vial of any one of claims 160, 164-176 and 178, wherein within each scFv comprising the VH and the VL of the anti-5T4 antibody, the VH is positioned at the N-terminus of the VL.

180. The pharmaceutical formulation or the vial of any one of claims 160, 167-168, 171, 172, 174, 175, 177, and 179, wherein the Fab fragment comprising the VH and the VL of the anti-NKG2D antibody is not positioned between an antigen-binding site and the Fc or the portion thereof.

181. The pharmaceutical formulation or the vial of any one of claims 161, 163, 165-167, 170, 172, 173, 175, 176, and 178, wherein no Fab fragment comprising the VH and the VL of the anti-5T4 antibody is positioned between an antigen-binding site and the Fc or the portion thereof.

182. The pharmaceutical formulation or the vial of any one of claims 159-181, wherein the first antigen-binding site binds human NKG2D.

183. The pharmaceutical formulation or the vial of any one of claims 159-182, wherein the second antigen-binding site binds human 5T4.

184. The pharmaceutical formulation or the vial of any one of claims 159-183, wherein the second antigen-binding site binds human 5T4 within an LRR1 domain.

185. The pharmaceutical formulation or the vial of any one of claims 159-184, wherein the first antigen-binding site comprising the VH and the VL of the anti-NKG2D antibody comprises a VH comprising CDR1, CDR2, and CDR3 comprising the amino acid sequences of SEQ ID NOs: 81, 82, and 112, respectively, and a VL comprising CDR1, CDR2, and CDR3 comprising the amino acid sequences of SEQ ID NOs: 86, 77, and 87, respectively.

186. The pharmaceutical formulation of any one of claims 119-143 and 158 or the vial of any one of claims 144-158, wherein the protein comprises:
(a) a first antigen-binding site comprising a VH and a VL of an anti-NKG2D antibody, wherein the VH comprises CDR1, CDR2, and CDR3 sequences represented by the amino acid sequences of SEQ ID NOs: 81, 82, and 112, respectively, and the VL comprises CDR1, CDR2, and CDR3 sequences represented by the amino acid sequences of SEQ ID NOs: 86, 77, and 87, respectively;
(b) a second antigen-binding site comprising a VH and a VL of an anti-5T4 antibody, wherein the VH comprises CDR1, CDR2, and CDR3 sequences represented by the amino acid sequences of SEQ ID NOs: 138, 139, and 140, respectively, and the VL comprises CDR1, CDR2, and CDR3 sequences represented by the amino acid sequences of SEQ ID NOs: 141, 142, and 143, respectively; and
(c) an antibody Fc domain or a portion thereof sufficient to bind CD16.

187. The pharmaceutical formulation or the vial of any one of claims 159-185, wherein the first antigen-binding site comprising the VH and the VL of the anti-NKG2D antibody comprises a VH comprising CDR1, CDR2, and CDR3 sequences represented by the amino acid sequences of SEQ ID NOs: 81, 82, and 97, respectively, and a VL comprising CDR1, CDR2, and CDR3 sequences represented by the amino acid sequences of SEQ ID NOs: 86, 77, and 87, respectively.

188. The pharmaceutical formulation of any one of claims 119-143 and 158 or the vial of any one of claims 144-158, wherein the protein comprises:
(a) a first antigen-binding site comprising a VH and a VL of an anti-NKG2D antibody, wherein the VH comprises CDR1, CDR2, and CDR3 sequences represented by the amino acid sequences of SEQ ID NOs: 81, 82, and 97, respectively, and the VL comprises CDR1, CDR2, and CDR3 sequences represented by the amino acid sequences of SEQ ID NOs: 86, 77, and 87, respectively;
(b) a second antigen-binding site comprising a VH and a VL of an anti-5T4 antibody, wherein the VH comprises CDR1, CDR2, and CDR3 sequences represented by the amino acid sequences of SEQ ID NOs: 138, 139, and 140, respectively, and the VL comprises CDR1, CDR2, and CDR3 sequences represented by the amino acid sequences of SEQ ID NOs: 141, 142, and 143, respectively; and
(c) an antibody Fc domain, comprising a first antibody Fc domain polypeptide or a portion thereof sufficient to bind CD16, and a second antibody Fc domain polypeptide or a portion thereof sufficient to bind CD16; and
wherein the VH or the VL of the anti-NKG2D antibody is fused to the N-terminus of the first antibody Fc domain polypeptide or portion thereof sufficient to bind CD16, and the VH or the VL of the anti-5T4 antibody is fused to the N-terminus of the second antibody Fc domain polypeptide or portion thereof sufficient to bind CD16.

189. The pharmaceutical formulation or the vial of any one of claims 159-188, wherein the first antigen-binding site comprises a VH comprising an amino acid sequence at least 90%, at least 91%, at least 92%, at least 93%, at least 94%, at least 95%, at least 96%, at least 97%, at least 98%, at least 99% or 100%, identical to SEQ ID NO:95 and a VL comprising an amino acid sequence at least 90%, at least 91%, at least 92%, at least 93%, at least 94%, at least 95%, at least 96%, at least 97%, at least 98%, at least 99% or 100%, identical to SEQ ID NO:85.

190. The pharmaceutical formulation or the vial of any one of claims 159-189, wherein the first antigen-binding site comprises a VH comprising an amino acid sequence of SEQ ID NO:95 and a VL comprising an amino acid sequence of SEQ ID NO:85.

191. The pharmaceutical formulation or the vial of any one of claims 159-190, wherein the second antigen-binding site comprises a VH at least 90%, at least 91%, at least 92%, at least 93%, at least 94%, at least 95%, at least 96%, at least 97%, at least 98%, at least 99% or 100%, identical to SEQ ID NO:144 and a VL at least 90%, at least 91%, at least 92%, at least 93%, at least 94%, at least 95%, at least 96%, at least 97%, at least 98%, at least 99% or 100%, identical to SEQ ID NO:145.

192. The pharmaceutical formulation or the vial of any one of claims 159-190, wherein the second antigen-binding site comprises a VH at least 90%, at least 91%, at least 92%, at least 93%, at least 94%, at least 95%, at least 96%, at least 97%, at least 98%, at least 99% or 100%, identical to SEQ ID NO:263 and a VL at least 90%, at least 91%, at least 92%, at least 93%, at least 94%, at least 95%, at least 96%, at least 97%, at least 98%, at least 99% or 100%, identical to SEQ ID NO:145.

193. The pharmaceutical formulation or the vial of claim 191 or 192, wherein the second antigen-binding site comprises a VH with a G44C substitution relative to SEQ ID NO:144 or SEQ ID NO:263 and a VL with a G100C substitution relative to SEQ ID NO:145, numbered under the Kabat numbering scheme.

194. The pharmaceutical formulation or the vial of any one of claims 159-192, wherein the second antigen-binding site comprises a VH comprising the amino acid sequence of SEQ ID NO:144 and a VL comprising the amino acid sequence of SEQ ID NO:145, or a VH comprising an amino acid sequence at least 90%, at least 91%, at least 92%, at least 93%, at least 94%, at least 95%, at least 96%, at least 97%, at least 98%, at least 99% or 100%, identical to SEQ ID NO:146 and a VL comprising an amino acid sequence at least 90%, at least 91%, at least 92%, at least 93%, at least 94%, at least 95%, at least 96%, at least 97%, at least 98%, at least 99% or 100%, identical to SEQ ID NO:147.

195. The pharmaceutical formulation or the vial of any one of claims 159-194, wherein the second antigen-binding site comprises a VH comprising the amino acid sequence of SEQ ID NO:146 and a VL comprising the amino acid sequence of SEQ ID NO:147.

196. The pharmaceutical formulation or the vial of any one of 159-192 and 194, wherein the second antigen-binding site comprises a VH comprising the amino acid sequence of SEQ ID NO:144 and a VL comprising the amino acid sequence of SEQ ID NO:145.

197. The pharmaceutical formulation or the vial of any one of claims 159, 160, 167-169, 171-172, 174-175, 177, 179-180, and 182-195, wherein the second antigen-binding site comprises a single-chain fragment variable (scFv), and wherein the scFv comprises a VH comprising the amino acid sequence of SEQ ID NO:146 and a VL comprising the amino acid sequence of SEQ ID NO:147.

198. The pharmaceutical formulation or the vial of any one of claims 159, 160, 167-169, 171-172, 174-175, 177, 179-180, and 182-195, wherein the second antigen-binding site comprises a single-chain fragment variable (scFv), and wherein the scFv comprises an amino acid sequence at least 90%, at least 91%, at least 92%, at least 93%, at least 94%, at least 95%, at least 96%, at least 97%, at least 98%, at least 99% or 100%, identical to a sequence selected from the group consisting of SEQ ID NOs: 148 and 149.

199. The pharmaceutical formulation or the vial of any one of claims 159, 160, 167-169, 171-172, 174-175, 177, 179-180, 182-195 and 197-198, wherein the second antigen-binding site comprises an scFv and the scFv comprises an amino acid sequence at least 90%, at least 91%, at least 92%, at least 93%, at least 94%, at least 95%, at least 96%, at least 97%, at least 98%, at least 99% or 100%, identical to SEQ ID NO:148.

200. The pharmaceutical formulation or the vial of any one of claims 159, 160, 167-169, 171-172, 174-175, 177, 179-180, 182-195 and 197-199, wherein the second antigen-binding site comprises an scFv and the scFv comprises an amino acid sequence of SEQ ID NO:148.

201. The pharmaceutical formulation or the vial of any one of claims 159, 160, 167-169, 171-172, 174-175, 177, 179-180, 182-195 and 197-200, wherein the protein comprises an amino acid sequence of SEQ ID NO:198.

202. A pharmaceutical formulation comprising:
a. a protein comprising:
i. a first antigen-binding site comprising a VH and a VL of an anti-NKG2D antibody, wherein the VH comprises the amino acid sequence of SEQ ID NO:95 and the VL comprises the amino acid sequence of SEQ ID NO:85;
ii. a second antigen-binding site comprising a VH and a VL of an anti-5T4 antibody, wherein the VH comprises the amino acid sequence of SEQ ID NO:146 and the VL comprises the amino acid sequence of SEQ ID NO:147; and
iii. an antibody Fc domain, comprising a first antibody Fc domain polypeptide or a portion thereof sufficient to bind CD16, and a second antibody Fc domain polypeptide or a portion thereof sufficient to bind CD16;
b. a buffer comprising citrate;
c. sucrose; and
d. a polysorbate,
wherein the pH of the pharmaceutical formulation is 6.0 to 7.0.

203. A pharmaceutical formulation comprising:
a. a protein comprising:
i. a first antigen-binding site comprising a VH and a VL of an anti-NKG2D antibody, wherein the VH comprises the amino acid sequence of SEQ ID NO:95 and the VL comprises the amino acid sequence of SEQ ID NO:85;
ii. a second antigen-binding site comprising the amino acid sequence of SEQ ID NO:148; and
iii. an antibody Fc domain, comprising a first antibody Fc domain polypeptide or a portion thereof sufficient to bind CD16, and a second antibody Fc domain polypeptide or a portion thereof sufficient to bind CD16;
b. a buffer comprising citrate;
c. sucrose; and
d. a polysorbate,
wherein the pH of the pharmaceutical formulation is 6.0 to 7.0.

204. A vial comprising a pharmaceutical formulation comprising:
a. a protein comprising:
i. a first antigen-binding site comprising a VH and a VL of an anti-NKG2D antibody, wherein the VH comprises the amino acid sequence of SEQ ID NO:95 and the VL comprises the amino acid sequence of SEQ ID NO:85;
ii. a second antigen-binding site comprising a VH and a VL of an anti-5T4 antibody, wherein the VH comprises the amino acid sequence of SEQ ID NO:146 and the VL comprises the amino acid sequence of SEQ ID NO:147; and
iii. an antibody Fc domain, comprising a first antibody Fc domain polypeptide or a portion thereof sufficient to bind CD16, and a second antibody Fc domain polypeptide or a portion thereof sufficient to bind CD16;
b. a buffer comprising citrate;
c. sucrose; and
d. a polysorbate,
wherein the pH of the pharmaceutical formulation is 6.0 to 7.0.

205. A vial comprising a pharmaceutical formulation comprising:
a. a protein comprising:
i. a first antigen-binding site comprising a VH and a VL of an anti-NKG2D antibody, wherein the VH comprises the amino acid sequence of SEQ ID NO:95 and the VL comprises the amino acid sequence of SEQ ID NO:85;
ii. a second antigen-binding site comprising the amino acid sequence of SEQ ID NO:148; and
iii. an antibody Fc domain, comprising a first antibody Fc domain polypeptide or a portion thereof sufficient to bind CD16, and a second antibody Fc domain polypeptide or a portion thereof sufficient to bind CD16;
b. a buffer comprising citrate;
c. sucrose; and
d. a polysorbate,
wherein the pH of the pharmaceutical formulation is 6.0 to 7.0.

206. The pharmaceutical formulation of any one of claims 119-143 and 158 or the vial of any one of claims 144-158, wherein the protein comprises:
(a) a first antigen-binding site comprising a heavy chain variable domain (VH) and a light chain variable domain (VL) of an anti-NKG2D antibody;
(b) a second antigen-binding site comprising a VH comprising an CDR1, a CDR2, and a CDR3 sequence selected from Table 2 and a VL comprising a CDR1, a CDR2, and a CDR3 sequence selected from Table 2, or a VH comprising a CDR1, a CDR2, and a CDR3 sequence selected from Table 12 and a VL comprising a CDR1, a CDR2, and a CDR3 comprising the amino acid sequences of SEQ ID NOs: 189, 190, and 143, respectively; and
(c) an antibody Fc domain or a portion thereof sufficient to bind CD16.

207. The pharmaceutical formulation or the vial of any one of claims 159-206, wherein the antibody Fc domain is a human IgG1 antibody Fc domain.

208. The pharmaceutical formulation or the vial of claim 207, wherein the antibody Fc domain or the portion thereof comprises an amino acid sequence at least 90%, at least 91%, at least 92%, at least 93%, at least 94%, at least 95%, at least 96%, at least 97%, at least 98%, at least 99% or 100%, identical to SEQ ID NO:118.

209. The pharmaceutical formulation or the vial of claim 207 or claim 208, wherein at least one polypeptide chain of the antibody Fc domain comprises one or more mutations, relative to SEQ ID NO:118, at one or more positions selected from Q347, Y349, L351, 5354, E356, E357, K360, Q362, S364, T366, L368, K370, N390, K392, T394, D399, S400, D401, F405, Y407, K409, T411, and K439, numbered according to the EU numbering system.

210. The pharmaceutical formulation or the vial of any one of claims 207-209, wherein at least one polypeptide chain of the antibody Fc domain comprises one or more mutations, relative to SEQ ID NO:118, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, F405L, Y407A, Y407I, Y407V, K409F, K409W, K409D, K409R, T411D, T411E, K439D, and K439E, numbered according to the EU numbering system.

211. The pharmaceutical formulation or the vial of any one of claims 207-210, wherein one polypeptide chain of the antibody heavy chain constant region comprises one or more mutations, relative to SEQ ID NO:118, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:118, at one or more positions selected from Q347, Y349, L351, S354, E356, E357, S364, T366, L368, K370, N390, K392, T394, D399, D401, F405, Y407, K409, T411, and K439, numbered according to the EU numbering system.

212. The pharmaceutical formulation or the vial of claim 211, wherein one polypeptide chain of the antibody heavy chain constant region comprises K360E and K409W substitutions relative to SEQ ID NO:118; and the other polypeptide chain of the antibody heavy chain constant region comprises Q347R, D399V and F405T substitutions relative to SEQ ID NO:118, numbered according to the EU numbering system.

213. The pharmaceutical formulation or the vial of claim 211, wherein one polypeptide chain of the antibody heavy chain constant region comprises an F405L substitution relative to SEQ ID NO:118; and the other polypeptide chain of the antibody heavy chain constant region comprises a K409R substitution relative to SEQ ID NO:118, numbered according to the EU numbering system.

214. The pharmaceutical formulation or the vial of any one of claims 211-213, wherein one polypeptide chain of the antibody heavy chain constant region comprises a Y349C substitution relative to SEQ ID NO:118; and the other polypeptide chain of the antibody heavy chain constant region comprises an S354C substitution relative to SEQ ID NO:118, numbered according to the EU numbering system.

215. The pharmaceutical formulation of any one of claims 119-143 and 158 or the vial of any one of claims 144-158, wherein the protein comprises:
(i) a first polypeptide comprising an amino acid sequence at least 90%, at least 91%, at least 92%, at least 93%, at least 94%, at least 95%, at least 96%, at least 97%, at least 98%, at least 99% or 100%, identical to SEQ ID NO:198;
(ii) a second polypeptide comprising an amino acid sequence at least 90%, at least 91%, at least 92%, at least 93%, at least 94%, at least 95%, at least 96%, at least 97%, at least 98%, at least 99% or 100%, identical to SEQ ID NO:199; and
(ii) a third polypeptide comprising an amino acid sequence at least 90%, at least 91%, at least 92%, at least 93%, at least 94%, at least 95%, at least 96%, at least 97%, at least 98%, at least 99% or 100%, identical to SEQ ID NO:200.

216. A method of enhancing tumor cell death, the method comprising exposing the tumor cell and a natural killer cell to an effective amount of the protein of any one of claims 1-112, the trispecific antibody of any one of claims 113-116, the pharmaceutical composition of claim 117, or the pharmaceutical formulation of any one of claims 119-143, 158-203, and 206-215.

217. A method of enhancing cancer-associated fibroblast (CAF) cell death, the method comprising exposing the CAF and a natural killer cell to an effective amount of the protein of any one of claims 1-112, the trispecific antibody of any one of claims 113-116, the pharmaceutical composition of claim 117, or the pharmaceutical formulation of any one of claims 119-143, 158-203, and 206-215.

218. A method of treating cancer, the method comprising administering to a subject in need thereof an effective amount of the protein of any one of claims 1-112, the trispecific antibody of any one of claims 113-116, the pharmaceutical composition of claim 117, or the pharmaceutical formulation of any one of claims 119-143, 158-203, and 206-215.

219. The method of claim 218, wherein the cancer is selected from the group consisting of colorectal cancer, ovarian cancer, non-small cell lung cancer, renal cancer, breast cancer, endometrial cancer, squamous cell carcinoma, head and neck squamous cell carcinoma, uterine cancer, pancreatic cancer, mesothelioma, and gastric cancer.

220. The method of claim 219, wherein the cancer is hormone receptor positive (HR+) breast cancer.

221. The method of any one of claims 218-220, wherein the cancer is a metastatic cancer.

222. The method of any one of claims 218-221, wherein the subject is refractory to chemotherapy.

223. The method of any one of claims 218-222, wherein the method increases overall survival and/or progression free survival in the subject.

224. The method of any one of claims 218-223, wherein 5T4 is expressed by cancer cells.

225. The method of any one of claims 218-224, wherein 5T4 is expressed by cancer-associated fibroblasts.

226. The method of any one of claims 224-225, wherein 5T4 is expressed at high levels relative to normal cells.

227. The method of any one of claims 224-225, wherein 5T4 is expressed at low levels relative to normal cells.

228. The protein according to any one of claims 1-112, wherein the protein is a purified protein.

229. The trispecific antibody according to any one of claims 113-116, wherein the trispecific antibody is a purified trispecific antibody.

230. The protein of claim 228 or the trispecific antibody of claim 229, wherein the protein or the trispecific antibody is purified using a method selected from the group consisting of: centrifugation, depth filtration, cell lysis, homogenization, freeze-thawing, affinity purification, gel filtration, ion exchange chromatography, hydrophobic interaction exchange chromatography, and mixed-mode chromatography.


SEQUENCE LISTING (552 sequences)

SEQ ID NO: 1 - protein, 117 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 9 aa, synthetic construct
GSFSGYYWS

SEQ ID NO: 3 - protein, 16 aa, synthetic construct
EIDHSGSTNYNPSLKS

SEQ ID NO: 4 - protein, 11 aa, synthetic construct
ARARGPWSFDP

SEQ ID NO: 5 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 6 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 7 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 8 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 9 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 10 - protein, 117 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 11 - protein, 107 aa, synthetic construct
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 12 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 13 - protein, 107 aa, synthetic construct
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 14 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 15 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 16 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 17 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 18 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 19 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 20 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 21 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 22 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 23 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 24 - protein, 107 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 25 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 26 - protein, 125 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 27 - protein, 9 aa, synthetic construct
GTFSSYAIS

SEQ ID NO: 28 - protein, 5 aa, synthetic construct
SYAIS

SEQ ID NO: 29 - protein, 17 aa, synthetic construct
GIIPIFGTANYAQKFQG

SEQ ID NO: 30 - protein, 18 aa, synthetic construct
ARGDSSIRHAYYYYGMDV

SEQ ID NO: 31 - protein, 16 aa, synthetic construct
GDSSIRHAYYYYGMDV

SEQ ID NO: 32 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 33 - protein, 17 aa, synthetic construct
KSSQSVLYSSNNKNYLA

SEQ ID NO: 34 - protein, 7 aa, synthetic construct
WASTRES

SEQ ID NO: 35 - protein, 9 aa, synthetic construct
QQYYSTPIT

SEQ ID NO: 36 - protein, 121 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 37 - protein, 11 aa, synthetic construct
GSISSSSYYWG

SEQ ID NO: 38 - protein, 7 aa, synthetic construct
SSSYYWG

SEQ ID NO: 39 - protein, 16 aa, synthetic construct
SIYYSGSTYYNPSLKS

SEQ ID NO: 40 - protein, 13 aa, synthetic construct
ARGSDRFHPYFDY

SEQ ID NO: 41 - protein, 11 aa, synthetic construct
GSDRFHPYFDY

SEQ ID NO: 42 - protein, 107 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 43 - protein, 11 aa, synthetic construct
RASQSVSRYLA

SEQ ID NO: 44 - protein, 7 aa, synthetic construct
DASNRAT

SEQ ID NO: 45 - protein, 9 aa, synthetic construct
QQFDTWPPT

SEQ ID NO: 46 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 47 - protein, 126 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 48 - protein, 19 aa, synthetic construct
ARRGRKASGSFYYYYGMDV

SEQ ID NO: 49 - protein, 113 aa, synthetic construct [sequence omitted]

SEQ ID NO: 50 - protein, 17 aa, synthetic construct
ESSQSLLNSGNQKNYLT

SEQ ID NO: 51 - protein, 9 aa, synthetic construct
QNDYSYPYT

SEQ ID NO: 52 - protein, 126 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 53 - protein, 9 aa, synthetic construct
YTFTSYYMH

SEQ ID NO: 54 - protein, 5 aa, synthetic construct
SYYMH

SEQ ID NO: 55 - protein, 17 aa, synthetic construct
IINPSGGSTSYAQKFQG

SEQ ID NO: 56 - protein, 19 aa, synthetic construct
ARGAPNYGDTTHDYYYMDV

SEQ ID NO: 57 - protein, 17 aa, synthetic construct
GAPNYGDTTHDYYYMDV

SEQ ID NO: 58 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 59 - protein, 11 aa, synthetic construct
RASQSVSSNLA

SEQ ID NO: 60 - protein, 7 aa, synthetic construct
GASTRAT

SEQ ID NO: 61 - protein, 9 aa, synthetic construct
QQYDDWPFT

SEQ ID NO: 62 - protein, 124 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 63 - protein, 9 aa, synthetic construct
YTFTGYYMH

SEQ ID NO: 64 - protein, 5 aa, synthetic construct
GYYMH

SEQ ID NO: 65 - protein, 17 aa, synthetic construct
WINPNSGGTNYAQKFQG

SEQ ID NO: 66 - protein, 17 aa, synthetic construct
ARDTGEYYDTDDHGMDV

SEQ ID NO: 67 - protein, 15 aa, synthetic construct
DTGEYYDTDDHGMDV

SEQ ID NO: 68 - protein, 107 aa, synthetic construct
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 69 - protein, 9 aa, synthetic construct
QQDDYWPPT

SEQ ID NO: 70 - protein, 121 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 71 - protein, 9 aa, synthetic construct
FTFSSYAMS

SEQ ID NO: 72 - protein, 17 aa, synthetic construct
AISGSGGSTYYADSVKG

SEQ ID NO: 73 - protein, 14 aa, synthetic construct
AKDGGYYDSGAGDY

SEQ ID NO: 74 - protein, 12 aa, synthetic construct
DGGYYDSGAGDY

SEQ ID NO: 75 - protein, 107 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 76 - protein, 11 aa, synthetic construct
RASQGIDSWLA

SEQ ID NO: 77 - protein, 7 aa, synthetic construct
AASSLQS

SEQ ID NO: 78 - protein, 9 aa, synthetic construct
QQGVSYPRT

SEQ ID NO: 79 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 80 - protein, 9 aa, synthetic construct
FTFSSYSMN

SEQ ID NO: 81 - protein, 5 aa, synthetic construct
SYSMN

SEQ ID NO: 82 - protein, 17 aa, synthetic construct
SISSSSSYIYYADSVKG

SEQ ID NO: 83 - protein, 15 aa, synthetic construct
ARGAPMGAAAGWFDP

SEQ ID NO: 84 - protein, 13 aa, synthetic construct
GAPMGAAAGWFDP

SEQ ID NO: 85 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 86 - protein, 11 aa, synthetic construct
RASQGISSWLA

SEQ ID NO: 87 - protein, 9 aa, synthetic construct
QQGVSFPRT

SEQ ID NO: 88 - protein, 249 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPMGAAAGWFDPWGQGTLVTVSS

SEQ ID NO: 89 - protein, 125 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 90 - protein, 18 aa, synthetic construct
AREGAGFAYGMDYYYMDV

SEQ ID NO: 91 - protein, 16 aa, synthetic construct
EGAGFAYGMDYYYMDV

SEQ ID NO: 92 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 93 - protein, 11 aa, synthetic construct
RASQSVSSYLA

SEQ ID NO: 94 - protein, 9 aa, synthetic construct
QQSDNWPFT

SEQ ID NO: 95 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 96 - protein, 15 aa, synthetic construct
ARGAPIGAAAGWFDP

SEQ ID NO: 97 - protein, 13 aa, synthetic construct
GAPIGAAAGWFDP

SEQ ID NO: 98 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 99 - protein, 15 aa, synthetic construct
ARGAPQGAAAGWFDP

SEQ ID NO: 100 - protein, 13 aa, synthetic construct
GAPQGAAAGWFDP

SEQ ID NO: 101 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 102 - protein, 15 aa, synthetic construct
ARGAPLGAAAGWFDP

SEQ ID NO: 103 - protein, 13 aa, synthetic construct
GAPLGAAAGWFDP

SEQ ID NO: 104 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 105 - protein, 15 aa, synthetic construct
ARGAPFGAAAGWFDP

SEQ ID NO: 106 - protein, 13 aa, synthetic construct
GAPFGAAAGWFDP

SEQ ID NO: 107 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 108 - protein, 15 aa, synthetic construct
ARGAPVGAAAGWFDP

SEQ ID NO: 109 - protein, 13 aa, synthetic construct
GAPVGAAAGWFDP

SEQ ID NO: 110 - protein, 122 aa, synthetic construct
  VARIANT 102: note = M, L, I, V, Q, or F
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 111 - protein, 15 aa, synthetic construct
  VARIANT 6: note = M, L, I, V, Q, or F
ARGAPXGAAAGWFDP

SEQ ID NO: 112 - protein, 13 aa, synthetic construct
  VARIANT 4: note = M, L, I, V, Q, or F
GAPXGAAAGWFDP

SEQ ID NO: 113 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 114 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 115 - protein, 5 aa, synthetic construct
SYAMS

SEQ ID NO: 116 - protein, 115 aa, synthetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 117 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 118 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 20 aa, synthetic construct
GGGGSGGGGSGGGGSGGGGS

SEQ ID NO: 120 - protein, 40 aa, synthetic construct
  VARIANT 1..40: note = This sequence may encompass 1-20 GS repeating units
GSGSGSGSGSGSGSGSGSGSGSGSGSGSGSGSGSGSGSGS

SEQ ID NO: 121 - protein, 60 aa, synthetic construct
  VARIANT 1..60: note = This sequence may encompass 1-20 GGS repeating units
GGSGGSGGSGGSGGSGGSGGSGGSGGSGGSGGSGGSGGSGGSGGSGGSGGSGGSGGSGGS

SEQ ID NO: 122 - protein, 80 aa, synthetic construct
  VARIANT 1..80: note = This sequence may encompass 1-20 GGGS repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 123 - protein, 80 aa, synthetic construct
  VARIANT 1..80: note = This sequence may encompass 1-20 GGSG repeating units
GGSGGGSGGGSGGGSGGGSGGGSGGGSGGGSGGGSGGGSGGGSGGGSGGGSGGGSGGGSGGGSGGGSGGGSGGGSGGGSG

SEQ ID NO: 124 - protein, 100 aa, synthetic construct
  VARIANT 1..100: note = This sequence may encompass 1-20 GGSGG repeating units
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 125 - protein, 100 aa, synthetic construct
  VARIANT 1..100: note = This sequence may encompass 1-20 GGGGS repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 126 - protein, 20 aa, synthetic construct
GSGSGSGSGSGSGSGSGSGS

SEQ ID NO: 127 - protein, 30 aa, synthetic construct
GGSGGSGGSGGSGGSGGSGGSGGSGGSGGS

SEQ ID NO: 128 - protein, 40 aa, synthetic construct
GGGSGGGSGGGSGGGSGGGSGGGSGGGSGGGSGGGSGGGS

SEQ ID NO: 129 - protein, 40 aa, synthetic construct
GGSGGGSGGGSGGGSGGGSGGGSGGGSGGGSGGGSGGGSG

SEQ ID NO: 130 - protein, 50 aa, synthetic construct
GGSGGGGSGGGGSGGGGSGGGGSGGGGSGGGGSGGGGSGGGGSGGGGSGG

SEQ ID NO: 131 - protein, 50 aa, synthetic construct
GGGGSGGGGSGGGGSGGGGSGGGGSGGGGSGGGGSGGGGSGGGGSGGGGS

SEQ ID NO: 132 - protein, 15 aa, synthetic construct
GGGGSGGGGSGGGGS

SEQ ID NO: 133 - protein, 100 aa, synthetic construct; identical to SEQ ID NO: 125

SEQ ID NO: 134 - protein, 100 aa, synthetic construct; identical to SEQ ID NO: 124

SEQ ID NO: 135 - protein, 8 aa, synthetic construct
SGSGGGGS

SEQ ID NO: 136 - protein, 120 aa, synthetic construct
EVQLQESGGGLVKPGGSRRLSCAASRFTFSDFGMHWVRQAPEKGLEWIAYISSGSSTIYYADTVKGRFTISRDNAKNTLFLQMTSLRSEDTAMYYCASSQSYYRGTLDYWGQGTSVTVSS

SEQ ID NO: 137 - protein, 111 aa, synthetic construct
DIVLTQSPASLAVTLGQRATISCRASQSVTTSNYNYMHWFHQKPGQPPKLLIKFASNLESGVPARFSGSGSGTDFTLNIHPVEEEDIATYYCQHSWEIPWTFGGGTKLEIK

SEQ ID NO: 138 - protein, 7 aa, synthetic construct
RFTFSDF

SEQ ID NO: 139 - protein, 6 aa, synthetic construct
SSGSST

SEQ ID NO: 140 - protein, 11 aa, synthetic construct
SQSYYRGTLDY

SEQ ID NO: 141 - protein, 15 aa, synthetic construct
RASQSVTTSNYNYMH

SEQ ID NO: 142 - protein, 7 aa, synthetic construct
FASNLES

SEQ ID NO: 143 - protein, 9 aa, synthetic construct
QHSWEIPWT

SEQ ID NO: 144 - protein, 120 aa, synthetic construct
EVQLVESGGGLVKPGGSLRLSCAASRFTFSDFGMHWVRQAPGKGLEWVSYISSGSSTIYYADSVKGRFTISRDNAKNSLYLQMNSLRAEDTAVYYCASSQSYYRGTLDYWGQGTTVTVSS

SEQ ID NO: 145 - protein, 111 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGGGTKVEIK

SEQ ID NO: 146 - protein, 120 aa, synthetic construct
EVQLVESGGGLVKPGGSLRLSCAASRFTFSDFGMHWVRQAPGKCLEWVSYISSGSSTIYYADSVKGRFTISRDNAKNSLYLQMNSLRAEDTAVYYCASSQSYYRGTLDYWGQGTTVTVSS

SEQ ID NO: 147 - protein, 111 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIK

SEQ ID NO: 148 - protein, 251 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASRFTFSDFGMHWVRQAPGKCLEWVSYISSGSSTIYYADSVKGRFTISRDNAKNSLYLQMNSLRAEDTAVYYCASSQSYYRGTLDYWGQGTTVTVSS

SEQ ID NO: 149 - protein, 251 aa, synthetic construct
EVQLVESGGGLVKPGGSLRLSCAASRFTFSDFGMHWVRQAPGKCLEWVSYISSGSSTIYYADSVKGRFTISRDNAKNSLYLQMNSLRAEDTAVYYCASSQSYYRGTLDYWGQGTTVTVSSGGGGSGGGGSGGGGSGGGGSDIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIK

SEQ ID NO: 150 - protein, 115 aa, synthetic construct
QVQLVQSGAEVKKPGASVKVSCKASGYKFTDYYMDWVRQAPGQGLEWIGYIFPNDASTTYNEKFKGKATLTADKSTNTAYMELSSLRSEDTAVYYCARSRDADYWGQGTTVTVSS

SEQ ID NO: 151 - protein, 106 aa, synthetic construct
ENVLTQSPATLSLSPGERATLSCSAKSSVSYIHWYQQKPGQAPRLLIYDTSYLGSGIPARFSGSGSGTDYTLTISSLEPEDFAVYYCQQWSSYPYTFGGGTKVEIK

SEQ ID NO: 152 - protein, 7 aa, synthetic construct
GYKFTDY

SEQ ID NO: 153 - protein, 6 aa, synthetic construct
SRDADY

SEQ ID NO: 154 - protein, 10 aa, synthetic construct
SAKSSVSYIH

SEQ ID NO: 155 - protein, 7 aa, synthetic construct
DTSYLGS

SEQ ID NO: 156 - protein, 9 aa, synthetic construct
QQWSSYPYT

SEQ ID NO: 157 - protein, 115 aa, synthetic construct
EVQLVQSGAEVKKPGASVKVSCKASGYKFTDYYMDWVRQAPGQGLEWIGYIFPNDASTTYNEKFKGKATLTADKSTNTAYMELSSLRSEDTAVYYCARSRDADYWGQGTTVTVSS

SEQ ID NO: 158 - protein, 6 aa, synthetic construct
FPNDAS

SEQ ID NO: 159 - protein, 241 aa, synthetic construct
QVQLVQSGAEVKKPGASVKVSCKASGYKFTDYYMDWVRQAPGQCLEWIGYIFPNDASTTYNEKFKGKATLTADKSTNTAYMELSSLRSEDTAVYYCARSRDADYWGQGTTVTVSSGGGGSGGGGSGGGGSGGGGSENVLTQSPATLSLSPGERATLSCSAKSSVSYIHWYQQKPGQAPRLLIYDTSYLGSGIPARFSGSGSGTDYTLTISSLEPEDFAVYYCQQWSSYPYTFGCGTKVEIK

SEQ ID NO: 160 - protein, 241 aa, synthetic construct
EVQLVQSGAEVKKPGASVKVSCKASGYKFTDYYMDWVRQAPGQCLEWIGYIFPNDASTTYNEKFKGKATLTADKSTNTAYMELSSLRSEDTAVYYCARSRDADYWGQGTTVTVSSGGGGSGGGGSGGGGSGGGGSENVLTQSPATLSLSPGERATLSCSAKSSVSYIHWYQQKPGQAPRLLIYDTSYLGSGIPARFSGSGSGTDYTLTISSLEPEDFAVYYCQQWSSYPYTFGCGTKVEIK

SEQ ID NO: 161 - protein, 120 aa, synthetic construct
EVQLVESGGGLVKPGGSLRLSCEASGFTFSDYGMHWVRQAPGKGLEWLAYISSGSSTFYYADTVKGRFTISRDNAKNSLYLQMNSLRAEDTAVYYCGSSQSYYRGTMDYWGQGTTVTVSS

SEQ ID NO: 162 - protein, 111 aa, synthetic construct
DIQLTQSPSSLSASVGDRVTITCRASQGVSTSTYTYLHWFQQKPGKAPKLLIKFASNLESGVPSRFSGSGSGTDFTLTISSLQPEDFATYYCQHSWEIPWTFGGGTKVEIK

SEQ ID NO: 163 - protein, 7 aa, synthetic construct
GFTFSDY

SEQ ID NO: 164 - protein, 11 aa, synthetic construct
SQSYYRGTMDY

SEQ ID NO: 165 - protein, 15 aa, synthetic construct
RASQGVSTSTYTYLH

SEQ ID NO: 166 - protein, 120 aa, synthetic construct
EVQLVESGGGLVKPGGSLRLSCAASRFTFSDFGMHWVRQAPGKGLEWVSYISSGSSTIYYADTVKGRFTISRDNAKNSLYLQMNSLRAEDTAVYYCASSQSYYRGTLDYWGQGTTVTVSS

SEQ ID NO: 167 - protein, 251 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASRFTFSDFGMHWVRQAPGKCLEWVSYISSGSSTIYYADTVKGRFTISRDNAKNSLYLQMNSLRAEDTAVYYCASSQSYYRGTLDYWGQGTTVTVSS

SEQ ID NO: 168 - protein, 120 aa, synthetic construct
EVQLLESGGGLVQPGGSLRLSCAASRFTFSDFGMHWVRQAPGKGLEWIAYISSGSSTIYYADTVKGRFTISRDNAKNTLYLQMNSLRAEDTAVYYCASSQSYYRGTLDYWGQGTTVTVSS

SEQ ID NO: 169 - protein, 251 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLLESGGGLVQPGGSLRLSCAASRFTFSDFGMHWVRQAPGKCLEWIAYISSGSSTIYYADTVKGRFTISRDNAKNTLYLQMNSLRAEDTAVYYCASSQSYYRGTLDYWGQGTTVTVSS

SEQ ID NO: 170 - protein, 120 aa, synthetic construct
EVQLVESGGGLVQPGGSLRLSCAASRFTFSDFGMHWVRQAPGKGLEWVSYISSGSSTIYYADTVKGRFTISRDNAKNSLYLQMNSLRAEDTAVYYCASSQSYYRGTLDYWGQGTTVTVSS

SEQ ID NO: 171 - protein, 251 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVQPGGSLRLSCAASRFTFSDFGMHWVRQAPGKCLEWVSYISSGSSTIYYADTVKGRFTISRDNAKNSLYLQMNSLRAEDTAVYYCASSQSYYRGTLDYWGQGTTVTVSS

SEQ ID NO: 172 - protein, 120 aa, synthetic construct
QVQLVESGGGLVKPGGSLRLSCAASRFTFSDFGMHWVRQAPGKGLEWIAYISSGSSTIYYADTVKGRFTISRDNAKNSLYLQMNSLRAEDTAVYYCASSQSYYRGTLDYWGQGTTVTVSS

SEQ ID NO: 173 - protein, 251 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSQVQLVESGGGLVKPGGSLRLSCAASRFTFSDFGMHWVRQAPGKCLEWIAYISSGSSTIYYADTVKGRFTISRDNAKNSLYLQMNSLRAEDTAVYYCASSQSYYRGTLDYWGQGTTVTVSS

SEQ ID NO: 174 - protein, 120 aa, synthetic construct
EVQLVESGGGLVKPGGSLRLSCAASRFTFSDFGMHWVRQAPGKGLEWIAYISSGSSTIYYADTVKGRFTISRDNAKNSLYLQMNSLRAEDTAVYYCASSQSYYRGTLDYWGQGTTVTVSS

SEQ ID NO: 175 - protein, 251 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASRFTFSDFGMHWVRQAPGKCLEWIAYISSGSSTIYYADTVKGRFTISRDNAKNSLYLQMNSLRAEDTAVYYCASSQSYYRGTLDYWGQGTTVTVSS

SEQ ID NO: 176 - protein, 120 aa, synthetic construct
EVQLVESGGGLVQPGGSLRLSCAASGFTFSDYGMHWVRQAPGKGLEWVSYISSGSSTFYYADTVKGRFTISRDNAKNSLYLQMNSLRAEDTAVYYCGSSQSYYRGTMDYWGQGTTVTVSS

SEQ ID NO: 177 - protein, 111 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGGGTKVEIK

SEQ ID NO: 178 - protein, 15 aa, synthetic construct
RASQGVSTSTYTYMH

SEQ ID NO: 179 - protein, 251 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVQPGGSLRLSCAASGFTFSDYGMHWVRQAPGKCLEWVSYISSGSSTFYYADTVKGRFTISRDNAKNSLYLQMNSLRAEDTAVYYCGSSQSYYRGTMDYWGQGTTVTVSS

SEQ ID NO: 180 - protein, 120 aa, synthetic construct
QVQLVESGGGLVKPGGSLRLSCAASGFTFSDYGMHWVRQAPGKGLEWLAYISSGSSTFYYADTVKGRFTISRDNAKNSLYLQMNSLRAEDTAVYYCGSSQSYYRGTMDYWGQGTTVTVSS

SEQ ID NO: 181 - protein, 251 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSQVQLVESGGGLVKPGGSLRLSCAASGFTFSDYGMHWVRQAPGKCLEWLAYISSGSSTFYYADTVKGRFTISRDNAKNSLYLQMNSLRAEDTAVYYCGSSQSYYRGTMDYWGQGTTVTVSS

SEQ ID NO: 182 - protein, 120 aa, synthetic construct
EVQLLESGGGLVQPGGSLRLSCAASGFTFSDYGMHWVRQAPGKGLEWVSYISSGSSTFYYADTVKGRFTISRDNSKNTLYLQMNSLRAEDTAVYYCGSSQSYYRGTMDYWGQGTTVTVSS

SEQ ID NO: 183 - protein, 251 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLLESGGGLVQPGGSLRLSCAASGFTFSDYGMHWVRQAPGKCLEWVSYISSGSSTFYYADTVKGRFTISRDNSKNTLYLQMNSLRAEDTAVYYCGSSQSYYRGTMDYWGQGTTVTVSS

SEQ ID NO: 184 - protein, 117 aa, synthetic construct
EVQLQESGAEMVRPGASVKLSCKASGYTFTSYWMHWVKQRPGQGLEWIGKIDSSDSKTQYNQKFKDKATLTVDKSSITVYMELNSLTSEDSAVYYCARGGYLWFAYWGQGTLVTVSA

SEQ ID NO: 185 - protein, 111 aa, synthetic construct
DIVMTQSPASLAVSLGQRATISCRASQSVSTSTYSYMHWYQQKPGQSPKLLITYASNLESGVPARFSGSGSGTDFTLNIHPVEEEDIATYYCQHSWEIPWTFGGGTKLEIK

SEQ ID NO: 186 - protein, 7 aa, synthetic construct
GYTFTSY

SEQ ID NO: 187 - protein, 6 aa, synthetic construct
DSSDSK

SEQ ID NO: 188 - protein, 8 aa, synthetic construct
GGYLWFAY

SEQ ID NO: 189 - protein, 15 aa, synthetic construct
RASQSVSTSTYSYMH

SEQ ID NO: 190 - protein, 7 aa, synthetic construct
YASNLES

SEQ ID NO: 191 - protein, 117 aa, synthetic construct
QVQLVQSGAEVKKPGASVKVSCKASGYTFTSYWMHWVRQAPGQGLEWMGKIDSSDSKTQYNQKFKDRVTMTRDTSTSTVYMELSSLRSEDTAVYYCARGGYLWFAYWGQGTLVTVSS

SEQ ID NO: 192 - protein, 111 aa, synthetic construct
DIVMTQSPDSLAVSLGERATINCRASQSVSTSTYSYMHWYQQKPGQPPKLLIYYASNLESGVPDRFSGSGSGTDFTLTISSLQAEDVAVYYCQHSWEIPWTFGGGTKVEIK

SEQ ID NO: 193 - protein, 248 aa, synthetic construct
QVQLVQSGAEVKKPGASVKVSCKASGYTFTSYWMHWVRQAPGQGLEWMGKIDSSDSKTQYNQKFKDRVTMTRDTSTSTVYMELSSLRSEDTAVYYCARGGYLWFAYWGQGTLVTVSSGGGGSGGGGSGGGGSGGGGSDIVMTQSPDSLAVSLGERATINCRASQSVSTSTYSYMHWYQQKPGQPPKLLIYYASNLESGVPDRFSGSGSGTDFTLTISSLQAEDVAVYYCQHSWEIPWTFGGGTKVEIK

SEQ ID NO: 194 - protein, 248 aa, synthetic construct
DIVMTQSPDSLAVSLGERATINCRASQSVSTSTYSYMHWYQQKPGQPPKLLIYYASNLESGVPDRFSGSGSGTDFTLTISSLQAEDVAVYYCQHSWEIPWTFGGGTKVEIKGGGGSGGGGSGGGGSGGGGSQVQLVQSGAEVKKPGASVKVSCKASGYTFTSYWMHWVRQAPGQGLEWMGKIDSSDSKTQYNQKFKDRVTMTRDTSTSTVYMELSSLRSEDTAVYYCARGGYLWFAYWGQGTLVTVSS

SEQ ID NO: 195 - protein, 116 aa, synthetic construct
QVQLVQSGAEVKKPGASVKVSCKASGYTFTSYWMHWVRQAPGQGLEWMGKIDSSDSKTQYNQKFKDRVTMTVDTSSSTVYMELSSLRSEDTAVYYCARGGYLWFAYWGQGTLVTVS

SEQ ID NO: 196 - protein, 111 aa, synthetic construct
DIVMTQSPASLAVSLGERATINCRASQSVSTSTYSYMHWYQQKPGQPPKLLITYASNLESGVPDRFSGSGSGTDFTLTISSLQAEDVATYYCQHSWEIPWTFGGGTKVEIK

SEQ ID NO: 197 - protein, 420 aa, synthetic construct
MPGGCSRGPAAGDGRLRLARLALVLLGWVSSSSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 198 - protein, 479 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASRFTFSDFGMHWVRQAPGKCLEWVSYISSGSSTIYYADSVKGRFTISRDNAKNSLYLQMNSLRAEDTAVYYCASSQSYYRGTLDYWGQGTTVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 199 - protein, 451 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 200 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 479 aa, synthetic construct
EVQLVESGGGLVKPGGSLRLSCAASRFTFSDFGMHWVRQAPGKCLEWVSYISSGSSTIYYADSVKGRFTISRDNAKNSLYLQMNSLRAEDTAVYYCASSQSYYRGTLDYWGQGTTVTVSSGGGGSGGGGSGGGGSGGGGSDIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 202 - protein, 251 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCEASGFTFSDYGMHWVRQAPGKCLEWLAYISSGSSTFYYADTVKGRFTISRDNAKNSLYLQMNSLRAEDTAVYYCGSSQSYYRGTMDYWGQGTTVTVSS

SEQ ID NO: 203 - protein, 7 aa, synthetic construct
GYTFGSY

SEQ ID NO: 204 - protein, 6 aa, synthetic construct
DASTEK

SEQ ID NO: 205 - protein, 8 aa, synthetic construct
GGYLWFQY

SEQ ID NO: 206 - protein, 7 aa, synthetic construct
GYLFTSY

SEQ ID NO: 207 - protein, 6 aa, synthetic construct
SVSDAK

SEQ ID NO: 208 - protein, 8 aa, synthetic construct
GGYLWFKY

SEQ ID NO: 209 - protein, 6 aa, synthetic construct
DARSAK

SEQ ID NO: 210 - protein, 7 aa, synthetic construct
GYRFTSY

SEQ ID NO: 211 - protein, 6 aa, synthetic construct
DASSAK

SEQ ID NO: 212 - protein, 7 aa, synthetic construct
GYGFTSY

SEQ ID NO: 213 - protein, 6 aa, synthetic construct
DARTAK

SEQ ID NO: 214 - protein, 8 aa, synthetic construct
GGYLWYAY

SEQ ID NO: 215 - protein, 6 aa, synthetic construct
DASDAK

SEQ ID NO: 216 - protein, 8 aa, synthetic construct
GGYLWYHY

SEQ ID NO: 217 - protein, 8 aa, synthetic construct
GGYLWYSY

SEQ ID NO: 218 - protein, 7 aa, synthetic construct
GYSFTSY

SEQ ID NO: 219 - protein, 8 aa, synthetic construct
GGHLWYAY

SEQ ID NO: 220 - protein, 469 aa, synthetic construct
ENVLTQSPATLSLSPGERATLSCSAKSSVSYIHWYQQKPGQAPRLLIYDTSYLGSGIPARFSGSGSGTDYTLTISSLEPEDFAVYYCQQWSSYPYTFGCGTKVEIKGGGGSGGGGSGGGGSGGGGSQVQLVQSGAEVKKPGASVKVSCKASGYKFTDYYMDWVRQAPGQCLEWIGYIFPNDASTTYNEKFKGKATLTADKSTNTAYMELSSLRSEDTAVYYCARSRDADYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 221 - protein, 241 aa, synthetic construct
ENVLTQSPATLSLSPGERATLSCSAKSSVSYIHWYQQKPGQAPRLLIYDTSYLGSGIPARFSGSGSGTDYTLTISSLEPEDFAVYYCQQWSSYPYTFGCGTKVEIKGGGGSGGGGSGGGGSGGGGSQVQLVQSGAEVKKPGASVKVSCKASGYKFTDYYMDWVRQAPGQCLEWIGYIFPNDASTTYNEKFKGKATLTADKSTNTAYMELSSLRSEDTAVYYCARSRDADYWGQGTTVTVSS

SEQ ID NO: 222 - protein, 469 aa, synthetic construct
QVQLVQSGAEVKKPGASVKVSCKASGYKFTDYYMDWVRQAPGQCLEWIGYIFPNDASTTYNEKFKGKATLTADKSTNTAYMELSSLRSEDTAVYYCARSRDADYWGQGTTVTVSSGGGGSGGGGSGGGGSGGGGSENVLTQSPATLSLSPGERATLSCSAKSSVSYIHWYQQKPGQAPRLLIYDTSYLGSGIPARFSGSGSGTDYTLTISSLEPEDFAVYYCQQWSSYPYTFGCGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 223 - protein, 469 aa, synthetic construct
EVQLVQSGAEVKKPGASVKVSCKASGYKFTDYYMDWVRQAPGQCLEWIGYIFPNDASTTYNEKFKGKATLTADKSTNTAYMELSSLRSEDTAVYYCARSRDADYWGQGTTVTVSSGGGGSGGGGSGGGGSGGGGSENVLTQSPATLSLSPGERATLSCSAKSSVSYIHWYQQKPGQAPRLLIYDTSYLGSGIPARFSGSGSGTDYTLTISSLEPEDFAVYYCQQWSSYPYTFGCGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 224 - protein, 479 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASRFTFSDFGMHWVRQAPGKCLEWVSYISSGSSTIYYADTVKGRFTISRDNAKNSLYLQMNSLRAEDTAVYYCASSQSYYRGTLDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 225 - protein, 479 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLLESGGGLVQPGGSLRLSCAASRFTFSDFGMHWVRQAPGKCLEWIAYISSGSSTIYYADTVKGRFTISRDNAKNTLYLQMNSLRAEDTAVYYCASSQSYYRGTLDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 226 - protein, 479 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVQPGGSLRLSCAASRFTFSDFGMHWVRQAPGKCLEWVSYISSGSSTIYYADTVKGRFTISRDNAKNSLYLQMNSLRAEDTAVYYCASSQSYYRGTLDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 227 - protein, 479 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVQPGGSLRLSCAASRFTFSDFGMHWVRQAPGKCLEWIAYISSGSSTIYYADTVKGRFTISRDNAKNSLYLQMNSLRAEDTAVYYCASSQSYYRGTLDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 228 - protein, 120 aa, synthetic construct
EVQLVESGGGLVQPGGSLRLSCAASRFTFSDFGMHWVRQAPGKGLEWIAYISSGSSTIYYADTVKGRFTISRDNAKNSLYLQMNSLRAEDTAVYYCASSQSYYRGTLDYWGQGTTVTVSS

SEQ ID NO: 229 - protein, 251 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVQPGGSLRLSCAASRFTFSDFGMHWVRQAPGKCLEWIAYISSGSSTIYYADTVKGRFTISRDNAKNSLYLQMNSLRAEDTAVYYCASSQSYYRGTLDYWGQGTTVTVSS

SEQ ID NO: 230 - protein, 479 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSQVQLVESGGGLVKPGGSLRLSCAASRFTFSDFGMHWVRQAPGKCLEWIAYISSGSSTIYYADTVKGRFTISRDNAKNSLYLQMNSLRAEDTAVYYCASSQSYYRGTLDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 231 - protein, 479 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASRFTFSDFGMHWIRQAPGKCLEWVSYISSGSSTIYYADTVKGRFTISRDNAKNSLYLQMNSLRAEDTAVYYCASSQSYYRGTLDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 232 - protein, 121 aa, synthetic construct
EVQLVESGGGLVKPGGSLRLSCAASRFTFSDFGMHWIRQAPGKGLEWVSYISSGSSTIYYADTVKGRFTISRDNAKNSLYLQMNSLRAEDTAVYYCASSQSYYRGTLDYWGQGTTVTVSSG

SEQ ID NO: 233 - protein, 251 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASRFTFSDFGMHWIRQAPGKCLEWVSYISSGSSTIYYADTVKGRFTISRDNAKNSLYLQMNSLRAEDTAVYYCASSQSYYRGTLDYWGQGTTVTVSS

SEQ ID NO: 234 - protein, 479 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASRFTFSDFGMHWVRQAPGKCLEWIAYISSGSSTIYYADTVKGRFTISRDNAKNSLYLQMNSLRAEDTAVYYCASSQSYYRGTLDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 235 - protein, 479 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLLESGGGLVQPGGSLRLSCAASRFTFSDFGMHWVRQAPGKCLEWVSYISSGSSTIYYADTVKGRFTISRDNSKNTLYLQMNSLRAEDTAVYYCASSQSYYRGTLDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 236 - protein, 120 aa, synthetic construct
EVQLLESGGGLVQPGGSLRLSCAASRFTFSDFGMHWVRQAPGKGLEWVSYISSGSSTIYYADTVKGRFTISRDNSKNTLYLQMNSLRAEDTAVYYCASSQSYYRGTLDYWGQGTTVTVSS

SEQ ID NO: 237 - protein, 479 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCEASGFTFSDYGMHWVRQAPGKCLEWLAYISSGSSTFYYADTVKGRFTISRDNAKNSLYLQMNSLRAEDTAVYYCGSSQSYYRGTMDYWGQGTTVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 238 - protein, 479 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVQPGGSLRLSCAASGFTFSDYGMHWVRQAPGKCLEWVSYISSGSSTFYYADTVKGRFTISRDNAKNSLYLQMNSLRAEDTAVYYCGSSQSYYRGTMDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 239 - protein, 479 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCEASGFTFSDYGMHWVRQAPGKCLEWLAYISSGSSTFYYADTVKGRFTISRDNAKNSLYLQMNSLRAEDTAVYYCGSSQSYYRGTMDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 240 - protein, 479 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSQVQLVESGGGLVKPGGSLRLSCAASGFTFSDYGMHWVRQAPGKCLEWLAYISSGSSTFYYADTVKGRFTISRDNAKNSLYLQMNSLRAEDTAVYYCGSSQSYYRGTMDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 241 - protein, 479 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASGFTFSDYGMHWVRQAPGKCLEWVSYISSGSSTFYYADTVKGRFTISRDNAKNSLYLQMNSLRAEDTAVYYCGSSQSYYRGTMDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 242 - protein, 120 aa, synthetic construct
EVQLVESGGGLVKPGGSLRLSCEASGFTFSDYGMHWVRQAPGKGLEWVSYISSGSSTFYYADTVKGRFTISRDNAKNSLYLQMNSLRAEDTAVYYCGSSQSYYRGTMDYWGQGTTVTVSS

SEQ ID NO: 243 - protein, 251 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCEASGFTFSDYGMHWVRQAPGKCLEWVSYISSGSSTFYYADTVKGRFTISRDNAKNSLYLQMNSLRAEDTAVYYCGSSQSYYRGTMDYWGQGTTVTVSS

SEQ ID NO: 244 - protein, 479 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVQPGGSLRLSCAASGFTFSDYGMHWVRQAPGKCLEWLAYISSGSSTFYYADTVKGRFTISRDNAKNSLYLQMNSLRAEDTAVYYCGSSQSYYRGTMDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 245 - protein, 120 aa, synthetic construct
EVQLVESGGGLVQPGGSLRLSCAASGFTFSDYGMHWVRQAPGKGLEWLAYISSGSSTFYYADTVKGRFTISRDNAKNSLYLQMNSLRAEDTAVYYCGSSQSYYRGTMDYWGQGTTVTVSS

SEQ ID NO: 246 - protein, 251 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVQPGGSLRLSCAASGFTFSDYGMHWVRQAPGKCLEWLAYISSGSSTFYYADTVKGRFTISRDNAKNSLYLQMNSLRAEDTAVYYCGSSQSYYRGTMDYWGQGTTVTVSS

SEQ ID NO: 247 - protein, 120 aa, synthetic construct
EVQLVESGGGLVKPGGSLRLSCAASGFTFSDYGMHWIRQAPGKGLEWVSYISSGSSTFYYADTVKGRFTISRDNAKNSLYLQMNSLRAEDTAVYYCGSSQSYYRGTMDYWGQGTTVTVSS

SEQ ID NO: 248 - protein, 251 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASGFTFSDYGMHWIRQAPGKCLEWVSYISSGSSTFYYADTVKGRFTISRDNAKNSLYLQMNSLRAEDTAVYYCGSSQSYYRGTMDYWGQGTTVTVSS

SEQ ID NO: 249 - protein, 479 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLLESGGGLVQPGGSLRLSCAASGFTFSDYGMHWVRQAPGKCLEWLAYISSGSSTFYYADTVKGRFTISRDNAKNTLYLQMNSLRAEDTAVYYCGSSQSYYRGTMDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 250 - protein, 120 aa, synthetic construct
EVQLLESGGGLVQPGGSLRLSCAASGFTFSDYGMHWVRQAPGKGLEWLAYISSGSSTFYYADTVKGRFTISRDNAKNTLYLQMNSLRAEDTAVYYCGSSQSYYRGTMDYWGQGTTVTVSS

SEQ ID NO: 251 - protein, 251 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLLESGGGLVQPGGSLRLSCAASGFTFSDYGMHWVRQAPGKCLEWLAYISSGSSTFYYADTVKGRFTISRDNAKNTLYLQMNSLRAEDTAVYYCGSSQSYYRGTMDYWGQGTTVTVSS

SEQ ID NO: 252 - protein, 477 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPIGAAAGWFDPWGQGTLVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 253 - protein, 444 aa, synthetic construct
QVQLVQSGAEVKKPGASVKVSCKASGYKFTDYYMDWVRQAPGQGLEWIGYIFPNDASTTYNEKFKGKATLTADKSTNTAYMELSSLRSEDTAVYYCARSRDADYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 254 - protein, 213 aa, synthetic construct
ENVLTQSPATLSLSPGERATLSCSAKSSVSYIHWYQQKPGQAPRLLIYDTSYLGSGIPARFSGSGSGTDYTLTISSLEPEDFAVYYCQQWSSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255 - protein, 449 aa, synthetic construct
EVQLVESGGGLVKPGGSLRLSCEASGFTFSDYGMHWVRQAPGKGLEWLAYISSGSSTFYYADTVKGRFTISRDNAKNSLYLQMNSLRAEDTAVYYCGSSQSYYRGTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 256 - protein, 218 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 449 aa, synthetic construct; identical to SEQ ID NO: 255

SEQ ID NO: 258 - protein, 218 aa, synthetic construct
DIQLTQSPSSLSASVGDRVTITCRASQGVSTSTYTYLHWFQQKPGKAPKLLIKFASNLESGVPSRFSGSGSGTDFTLTISSLQPEDFATYYCQHSWEIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 259 - protein, 449 aa, synthetic construct
EVQLVESGGGLVKPGGSLRLSCAASRFTFSDFGMHWVRQAPGKGLEWVSYISSGSSTIYYADTVKGRFTISRDNAKNSLYLQMNSLRAEDTAVYYCASSQSYYRGTLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 260 - protein, 218 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 261 - protein, 449 aa, synthetic construct
EVQLVESGGGLVKPGGSLRLSCAASRFTFSDFGMHWVRQAPGKGLEWVSYISSGSSTIYYADSVKGRFTISRDNAKNSLYLQMNSLRAEDTAVYYCASSQSYYRGTLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 262 - protein, 218 aa, synthetic construct; identical to SEQ ID NO: 260

SEQ ID NO: 263 - protein, 120 aa, synthetic construct
  VARIANT 1: note = E or Q
  VARIANT 5: note = V or L
  VARIANT 13: note = K or Q
  VARIANT 48: note = V or I
  VARIANT 49: note = S or A
  VARIANT 63: note = S or T
  VARIANT 78: note = S or T
XVQLXESGGGLVXPGGSLRLSCAASRFTFSDFGMHWVRQAPGKGLEWXXYISSGSSTIYYADXVKGRFTISRDNAKNXLYLQMNSLRAEDTAVYYCASSQSYYRGTLDYWGQGTTVTVSS

SEQ ID NO: 264 - protein, 251 aa, synthetic construct
  VARIANT 1: note = E or Q
  VARIANT 5: note = V or L
  VARIANT 13: note = K or Q
  VARIANT 48: note = V or I
  VARIANT 49: note = S or A
  VARIANT 63: note = S or T
  VARIANT 78: note = S or T
XVQLXESGGGLVXPGGSLRLSCAASRFTFSDFGMHWVRQAPGKGLEWXXYISSGSSTIYYADXVKGRFTISRDNAKNXLYLQMNSLRAEDTAVYYCASSQSYYRGTLDYWGQGTTVTVSSGGGGSGGGGSGGGGSGGGGSDIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGGGTKVEIK

SEQ ID NO: 265 - protein, 251 aa, synthetic construct
  VARIANT 132: note = E or Q
  VARIANT 136: note = V or L
  VARIANT 144: note = K or Q
  VARIANT 179: note = V or I
  VARIANT 180: note = S or A
  VARIANT 194: note = S or T
  VARIANT 209: note = S or T
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGGGTKVEIKGGGGSGGGGSGGGGSGGGGSXVQLXESGGGLVXPGGSLRLSCAASRFTFSDFGMHWVRQAPGKGLEWXXYISSGSSTIYYADXVKGRFTISRDNAKNXLYLQMNSLRAEDTAVYYCASSQSYYRGTLDYWGQGTTVTVSS

SEQ ID NO: 266 - protein, 120 aa, synthetic construct
EVQLVESGGGLVKPGGSLRLSCAASGFTFSDYGMHWVRQAPGKGLEWVSYISSGSSTFYYADTVKGRFTISRDNAKNSLYLQMNSLRAEDTAVYYCGSSQSYYRGTMDYWGQGTTVTVSS

SEQ ID NO: 267 - protein, 111 aa, synthetic construct
DIQLTQSPSSLSASVGDRVTITCRASQGVSTSTYTYVHWFQQKPGKAPKLLIKFASNLESGVPSRFSGSGSGTDFTLTISSLQPEDFATYYCQHSWEIPWTFGGGTKVEIK

SEQ ID NO: 268 - protein, 15 aa, synthetic construct
RASQGVSTSTYTYVH

SEQ ID NO: 269 - protein, 120 aa, synthetic construct
EVQLVESGGGLVKPGGSLRLSCAASGFTFSDYGMHWVRQAPGKGLEWVSYISSGSSTFYYADTVKGRFTISRDNAKNSLYLQMNSLRAEDTAVYYCGSSQSYYRGTIDYWGQGTTVTVSS

SEQ ID NO: 270 - protein, 11 aa, synthetic construct
SQSYYRGTIDY

SEQ ID NO: 271 - protein, 251 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASGFTFSDYGMHWVRQAPGKCLEWVSYISSGSSTFYYADTVKGRFTISRDNAKNSLYLQMNSLRAEDTAVYYCGSSQSYYRGTMDYWGQGTTVTVSS

SEQ ID NO: 272 - protein, 251 aa, synthetic construct
DIQLTQSPSSLSASVGDRVTITCRASQGVSTSTYTYLHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASGFTFSDYGMHWVRQAPGKCLEWVSYISSGSSTFYYADTVKGRFTISRDNAKNSLYLQMNSLRAEDTAVYYCGSSQSYYRGTMDYWGQGTTVTVSS

SEQ ID NO: 273 - protein, 251 aa, synthetic construct
DIQLTQSPSSLSASVGDRVTITCRASQGVSTSTYTYVHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASGFTFSDYGMHWVRQAPGKCLEWVSYISSGSSTFYYADTVKGRFTISRDNAKNSLYLQMNSLRAEDTAVYYCGSSQSYYRGTMDYWGQGTTVTVSS

SEQ ID NO: 274 - protein, 251 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASGFTFSDYGMHWVRQAPGKCLEWVSYISSGSSTFYYADTVKGRFTISRDNAKNSLYLQMNSLRAEDTAVYYCGSSQSYYRGTIDYWGQGTTVTVSS

SEQ ID NO: 275 - protein, 251 aa, synthetic construct
DIQLTQSPSSLSASVGDRVTITCRASQGVSTSTYTYLHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASGFTFSDYGMHWVRQAPGKCLEWVSYISSGSSTFYYADTVKGRFTISRDNAKNSLYLQMNSLRAEDTAVYYCGSSQSYYRGTIDYWGQGTTVTVSS

SEQ ID NO: 276 - protein, 251 aa, synthetic construct
DIQLTQSPSSLSASVGDRVTITCRASQGVSTSTYTYVHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASGFTFSDYGMHWVRQAPGKCLEWVSYISSGSSTFYYADTVKGRFTISRDNAKNSLYLQMNSLRAEDTAVYYCGSSQSYYRGTIDYWGQGTTVTVSS

SEQ ID NO: 277 - protein, 251 aa, synthetic construct; identical to SEQ ID NO: 202

SEQ ID NO: 278 - protein, 251 aa, synthetic construct
DIQLTQSPSSLSASVGDRVTITCRASQGVSTSTYTYLHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCEASGFTFSDYGMHWVRQAPGKCLEWLAYISSGSSTFYYADTVKGRFTISRDNAKNSLYLQMNSLRAEDTAVYYCGSSQSYYRGTMDYWGQGTTVTVSS

SEQ ID NO: 279 - protein, 251 aa, synthetic construct
DIQLTQSPSSLSASVGDRVTITCRASQGVSTSTYTYVHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCEASGFTFSDYGMHWVRQAPGKCLEWLAYISSGSSTFYYADTVKGRFTISRDNAKNSLYLQMNSLRAEDTAVYYCGSSQSYYRGTMDYWGQGTTVTVSS

SEQ ID NO: 280 - DNA, 7537 bp, synthetic construct
  source 1..7537: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttgccaccatggacatgagagtgcccgctcaactgctgggactgctgctgctttggcttcctggcgctagatgcgacatccagctgacacagagccctagcagcctgtctgcctctgtgggcgacagagtgaccatcacctgtagagccagccagagcgtgaccaccagcaactacaactacatgcactggttccagcagaagcccggcaaggcccctaagctgctgatcaagttcgccagcaacctggaaagcggcgtgcccagcagattttctggcagcggctctggcaccgacttcaccctgaccatatctagcctgcagcctgaggacttcgccacctactactgtcagcacagctgggagatcccttggacctttggctgtggcaccaaggtggaaatcaaaggcggcggaggatctggcggaggtggaagcggaggcggaggaagcggtggcggcggatctgaagttcagctggttgaaagtggcggaggcctggttaagcctggcggatctctgagactgagctgtgccgcctccagattcaccttcagcgacttcggaatgcactgggtccgacaggcccctggcaaatgtctggaatgggtgtcctacatcagcagcggcagcagcaccatctactacgccgatagcgtgaagggcagattcacaatcagccgggacaacgccaagaacagcctgtacctgcagatgaactccctgagagccgaggacaccgccgtgtactattgtgccagcagccagtcctactaccggggcacactggattattggggccagggcacaaccgtgacagtgtccagcgccagcgataagacccacacctgtcctccatgtcctgctccagaactgctcggcggaccttccgtgttcctgtttcctccaaagcctaaggacaccctgatgatctccagaacacccgaagtgacctgcgtggtggtggatgtgtctcacgaggaccctgaagtgaagttcaattggtacgtggacggcgtggaagtgcacaatgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctcgcgagcctagagtgtataccttgcctccatgccgggacgagctgaccaagaatcaggtgtccctgacctgcctggtcaagggcttctacccttccgatatcgccgtggaatgggagagcaatggccagcctgagaacaactacaagaccacacctcctgtgctggtgtccgacggcagctttaccctgtacagcaagctgacagtggacaagagcagatggcagcagggcaacgtgttcagctgcagcgtgatgcacgaggccctgcacaaccactacacccagaagtccctgagcctgtctcctggatgataatctagaaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 281 - DNA, 1530 bp, synthetic construct
  source 1..1530: mol_type = other DNA
aagcttaccgccaccatggacatgcgcgtccccgcgcagctcctcggcctcctgctgctctggctccccggcgcccgctgcgacatccaactgacccagtccccgtcctccctgagcgcatctgtcggcgaccgggtgaccatcacctgccgggcctcgcagtccgtgaccacgtcgaactacaactacatgcactggttccagcagaagcccggtaaggcgcccaagctgctcatcaagttcgcgtccaacctggagtcgggcgtgccgtcgcggttctccggctccggcagcggcaccgacttcacgctgacgatctcgtccctccagcccgaggacttcgccacgtactactgccagcactcctgggagatcccgtggacgttcgggtgcggcaccaaggtcgagatcaagggaggcgggggcagtgggggcggaggctctgggggcggcggcagtggcggcggcggctcggaggtccagctggtagagtccggaggcggcctcgtgaagccgggcggctccctgcgcctgtcctgcgcggccagccgtttcaccttctcggacttcgggatgcattgggtgcgccaggccccaggcaagtgcctggagtgggtttcctacatctcgtccggctcgagtaccatctattacgccgacagcgtcaagggacgcttcacgatctcgcgggacaacgccaagaacagcctttacctgcagatgaacagcctgcgcgcggaggacaccgccgtctactactgcgcctcgtcccagagctattaccggggtaccctcgactactggggccaggggacgacggtgaccgtcagctccgccagcgacaagacccacacgtgccccccgtgcccggccccggagctgctgggaggcccgtccgtgttcctgttccccccgaagcccaaggacaccctgatgatctcgcgcaccccggaggtgacctgcgtagtcgtcgacgtgtcccacgaggaccccgaggtgaagttcaactggtacgtggacggcgtcgaggtccacaacgcgaagaccaagccgcgcgaggagcagtacaacagcacctaccgggtcgtgagcgtcctgaccgtgctgcaccaagactggctcaacggtaaggagtacaagtgtaaggtgagcaacaaggccctccctgccccgattgagaagaccatctcgaaggctaagggccagccgcgggagccccgggtgtacacgctcccgccctgccgggacgaactgaccaaaaaccaggtgtccctgacgtgtctggtgaagggcttctacccctccgacatcgccgtcgagtgggagtccaatggtcagcccgagaacaactacaagacgaccccgcccgtgctggtgagcgacgggtccttcaccctgtattccaagctcacggtcgacaagtcccgctggcagcagggcaacgtcttctcgtgctccgtgatgcacgaggccctccacaaccactacacccagaagtcactgtcactctcccccggctgataatctaga

SEQ ID NO: 282 - DNA, 7441 bp, synthetic construct
  source 1..7441: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcgccaccatgggctggtcctgcatcatcctgtttctggtggccacagccacaggcgtgcactctgaggtgcagctggttgaatctggcggcggacttgtgaagcctggcggatctctgagactgagctgtgccgccagcggcttcacctttagcagctacagcatgaactgggtccgacaggcccctggcaaaggccttgaatgggtgtccagcatcagcagcagctccagctacatctactacgccgacagcgtgaagggcagattcaccatcagccgggacaacgccaagaacagcctgtacctgcagatgaactccctgagagccgaggacaccgccgtgtactattgtgctagaggcgcccctattggagccgccgctggatggttcgatccttggggacagggaaccctggtcaccgtttcttctgccagcacaaagggccctagcgttttcccactggctcccagcagcaagagcacaagcggaggaacagctgccctgggctgtctggtcaaggactactttcctgagcctgtgaccgtgtcctggaacagcggagcactgactagcggcgtgcacacatttccagccgtgctgcaaagcagcggcctgtactctctgagcagcgtcgtgacagtgcctagcagctctctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcctgctcctatcgagaaaaccatcagcaaggccaagggccagcctcgcgaacctcaagtctgtacactgcctcctagccgggatgagctgaccgagaatcaggtgtccctgacctgtctcgtgaagggcttctacccctccgatatcgccgtggaatgggagagcaatggccagccagagaacaactacaagacaacccctcctgtgctggacagcgacggctcattcttcctgtacagctggctgaccgtggacaagtccagatggcagcagggcaacgtgttctcctgcagcgtgatgcacgaggccctgcacaaccactacacccagaagtccctgtctctgagcccaggctaaggatccaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 283 - DNA, 6727 bp, synthetic construct
  source 1..6727: mol_type = other DNA
ctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgccaccatggacatgagagttccagctcagctgctgggcctgctgctgctttggcttcctggcgctagatgcgacatccagatgacacagagccccagctccgtgtctgcctctgtgggagacagagtgaccatcacctgtagagccagccagggcatctcttcttggctggcctggtatcagcagaagcctggcaaggcccctaagctgctgatctatgccgctagctctctgcagtctggcgtgccctctagattttctggcagcggctctggcaccgacttcaccctgaccatatctagcctgcagcctgaggacttcgccacctactattgtcagcagggcgtgtcctttccacggacctttggcggcggaacaaaggtggaaatcaagcggacagtggccgctcctagcgtgttcatctttccacctagcgacgagcagctgaagtccggcacagcctctgttgtgtgcctgctgaacaacttctaccccagagaagccaaggtgcagtggaaggtggacaatgccctgcagagcggcaacagccaagagagcgtgacagagcaggacagcaaggactccacctacagcctgagcagcaccctgacactgagcaaggccgactacgagaagcacaaagtgtacgcctgcgaagtgacccaccagggcctttctagccctgtgaccaagagcttcaaccggggcgagtgttgaaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcg

SEQ ID NO: 284 - DNA, 7456 bp, synthetic construct
  source 1..7456: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttaccgccaccatggacatgagagtgcccgctcagctgctgggactgctgttgttgtggttgcctggcgctagatgcgaggtgcagctggttgaatctggcggcggacttgtgaagcctggcggatctctgagactgtcttgtgccgcctccggcttcaccttctccagctactctatgaactgggtccgacaggcccctggcaaaggactggaatgggtgtcctctatctcctccagctcctcctacatctactacgccgactccgtgaagggcagattcaccatcagccgggacaacgccaagaactccctgtacctgcagatgaacagcctgagagccgaggacaccgccgtgtactattgtgctagaggcgctcctatcggcgctgccgctggatggttcgatccttggggacagggaaccctggtcaccgtgtcctctgcttctaccaagggacccagcgtgttccctctggctccttccagcaagtctacctctggcggaacagctgctctgggctgcctggtcaaggactactttcctgagcctgtgaccgtgtcttggaactctggcgctctgacatccggcgtgcacacatttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaatgccaagaccaagcctagagaggaacagtacaactccacctacagagtggtgtccgtgctgaccgtgctgcaccaggattggctgaacggcaaagagtacaagtgcaaggtgtccaacaaggccctgcctgctccaatcgaaaagaccatctccaaggccaagggccagcctcgggaacctcaagtctgtaccttgcctcctagccgggatgagctgaccgagaatcaggtgtccctgacctgcctcgtgaagggattctacccctccgatatcgccgtggaatgggagtctaatggccagccagagaacaactacaagacaacccctcctgtgctggactccgacggctccttctttctgtactcctggctgacagtggacaagtccagatggcagcagggcaacgtgttctcctgctccgtgatgcacgaggccctgcacaatcactacacccagaagtccctgtctctgtcccctggctgatgatctagaaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 285 - DNA, 6745 bp, synthetic construct
  source 1..6745: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttaccgccaccatggacatgagagtgcccgctcagctgctgggactgctgttgttgtggttgcctggcgctagatgcgacatccagatgacccagtctccatcctccgtgtctgcctctgtgggcgacagagtgaccatcacctgtagagccagccagggcatctcttcttggctggcctggtatcagcagaagcctggcaaggctcccaagctgctgatctacgctgcttccagtctgcagtctggcgtgccctccagattttccggctctggctctggcaccgactttaccctgacaatctccagcctgcagcctgaggacttcgccacctactattgtcagcagggcgtgtcctttcctcggacctttggcggaggcaccaaggtggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgctctgcagtccggcaactcccaagagtccgtgaccgagcaggactccaaggactctacctacagcctgtcctccacactgaccctgtccaaggccgactacgagaagcacaaggtgtacgcctgcgaagtgacccatcagggcctgtctagccctgtgaccaagtctttcaaccggggcgagtgctgatgatctagaaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 286 - protein, 479 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLLESGGGLVQPGGSLRLSCAASGFTFSDYGMHWVRQAPGKCLEWVSYISSGSSTFYYADTVKGRFTISRDNSKNTLYLQMNSLRAEDTAVYYCGSSQSYYRGTMDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 287 - protein, 479 aa, synthetic construct
DIQLTQSPSSLSASVGDRVTITCRASQGVSTSTYTYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLVESGGGLVKPGGSLRLSCAASGFTFSDYGMHWIRQAPGKCLEWVSYISSGSSTFYYADTVKGRFTISRDNAKNSLYLQMNSLRAEDTAVYYCGSSQSYYRGTMDYWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 288 - protein, 249 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPIGAAAGWFDPWGQGTLVTVSS

SEQ ID NO: 289 - protein, 111 aa, synthetic construct
DIQLTQSPSSLSASVGDRVTITCRASQSVTTSNYNYLHWFQQKPGKAPKLLIKFASNLESGVPSRFSGSGSGTDFTLTISSLQPEDFATYYCQHSWEIPWTFGGGTKVEIK

SEQ ID NO: 290 - protein, 15 aa, synthetic construct
RASQSVTTSNYNYLH

SEQ ID NO: 291 - protein, 111 aa, synthetic construct
DIQLTQSPSSLSASVGDRVTITCRASQSVTTSNYNYVHWFQQKPGKAPKLLIKFASNLESGVPSRFSGSGSGTDFTLTISSLQPEDFATYYCQHSWEIPWTFGGGTKVEIK

SEQ ID NO: 292 - protein, 15 aa, synthetic construct
RASQSVTTSNYNYVH

SEQ ID NO: 293 - protein, 251 aa, synthetic construct
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSEVQLLESGGGLVQPGGSLRLSCAASRFTFSDFGMHWVRQAPGKCLEWVSYISSGSSTIYYADTVKGRFTISRDNSKNTLYLQMNSLRAEDTAVYYCASSQSYYRGTLDYWGQGTTVTVSS

SEQ ID NO: 294 - protein, 248 aa, synthetic construct
QVQLVQSGAEVKKPGASVKVSCKASGYTFTSYWMHWVRQAPGQCLEWMGKIDSSDSKTQYNQKFKDRVTMTRDTSTSTVYMELSSLRSEDTAVYYCARGGYLWFAYWGQGTLVTVSSGGGGSGGGGSGGGGSGGGGSDIVMTQSPDSLAVSLGERATINCRASQSVSTSTYSYMHWYQQKPGQPPKLLIYYASNLESGVPDRFSGSGSGTDFTLTISSLQAEDVAVYYCQHSWEIPWTFGCGTKVEIK

SEQ ID NO: 295 - protein, 248 aa, synthetic construct
DIVMTQSPDSLAVSLGERATINCRASQSVSTSTYSYMHWYQQKPGQPPKLLIYYASNLESGVPDRFSGSGSGTDFTLTISSLQAEDVAVYYCQHSWEIPWTFGCGTKVEIKGGGGSGGGGSGGGGSGGGGSQVQLVQSGAEVKKPGASVKVSCKASGYTFTSYWMHWVRQAPGQCLEWMGKIDSSDSKTQYNQKFKDRVTMTRDTSTSTVYMELSSLRSEDTAVYYCARGGYLWFAYWGQGTLVTVSS

SEQ ID NO: 296 - protein, 251 aa, synthetic construct
  VARIANT 1: note = E or Q
  VARIANT 5: note = V or L
  VARIANT 13: note = K or Q
  VARIANT 48: note = V or I
  VARIANT 49: note = S or A
  VARIANT 63: note = S or T
  VARIANT 78: note = S or T
XVQLXESGGGLVXPGGSLRLSCAASRFTFSDFGMHWVRQAPGKCLEWXXYISSGSSTIYYADXVKGRFTISRDNAKNXLYLQMNSLRAEDTAVYYCASSQSYYRGTLDYWGQGTTVTVSSGGGGSGGGGSGGGGSGGGGSDIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIK

SEQ ID NO: 297 - protein, 251 aa, synthetic construct
  VARIANT 132: note = E or Q
  VARIANT 136: note = V or L
  VARIANT 144: note = K or Q
  VARIANT 179: note = V or I
  VARIANT 180: note = S or A
  VARIANT 194: note = S or T
  VARIANT 209: note = S or T
DIQLTQSPSSLSASVGDRVTITCRASQSVTTSNYNYMHWFQQKPGKAPKLLIKFASNLESGVPSRFSGSGSGTDFTLTISSLQPEDFATYYCQHSWEIPWTFGCGTKVEIKGGGGSGGGGSGGGGSGGGGSXVQLXESGGGLVXPGGSLRLSCAASRFTFSDFGMHWVRQAPGKCLEWXXYISSGSSTIYYADXVKGRFTISRDNAKNXLYLQMNSLRAEDTAVYYCASSQSYYRGTLDYWGQGTTVTVSS

SEQ ID NO: 298 - protein, 5 aa, synthetic construct
GYYWS

SEQ ID NO: 299 - protein, 9 aa, synthetic construct
ARGPWSFDP

SEQ ID NO: 300 - protein, 11 aa, synthetic construct
RASQSISSWLA

SEQ ID NO: 301 - protein, 7 aa, synthetic construct
KASSLES

SEQ ID NO: 302 - protein, 9 aa, synthetic construct
QQYNSYPIT

SEQ ID NO: 303 - protein, 12 aa, synthetic construct
RASQSVSSSYLA

SEQ ID NO: 304 - protein, 7 aa, synthetic construct
GASSRAT

SEQ ID NO: 305 - protein, 9 aa, synthetic construct
QQYGSSPIT

SEQ ID NO: 306 - protein, 11 aa, synthetic construct
RASQSIGSWLA

SEQ ID NO: 307 - protein, 8 aa, synthetic construct
QQYHSFYT

SEQ ID NO: 308 - protein, 8 aa, synthetic construct
QQSNSYYT

SEQ ID NO: 309 - protein, 8 aa, synthetic construct
QQYNSYPT

SEQ ID NO: 310 - protein, 9 aa, synthetic construct
ARGPWGFDP

SEQ ID NO: 311 - protein, 11 aa, synthetic construct
RTSQSISSYLN

SEQ ID NO: 312 - protein, 9 aa, synthetic construct
QQSYDIPYT

SEQ ID NO: 313 - protein, 9 aa, synthetic construct
QQYGSFPIT

SEQ ID NO: 314 - protein, 9 aa, synthetic construct
QQSKEVPWT

SEQ ID NO: 315 - protein, 8 aa, synthetic construct
QQYNSFPT

SEQ ID NO: 316 - protein, 8 aa, synthetic construct
QQYDIYPT

SEQ ID NO: 317 - protein, 8 aa, synthetic construct
QQYDSYPT

SEQ ID NO: 318 - protein, 8 aa, synthetic construct
QQYGSFPT

SEQ ID NO: 319 - protein, 8 aa, synthetic construct
QQYQSFPT

SEQ ID NO: 320 - protein, 8 aa, synthetic construct
QQYSSFST

SEQ ID NO: 321 - protein, 8 aa, synthetic construct
QQYESYST

SEQ ID NO: 322 - protein, 8 aa, synthetic construct
QQYDSFIT

SEQ ID NO: 323 - protein, 8 aa, synthetic construct
QQYQSYPT

SEQ ID NO: 324 - protein, 8 aa, synthetic construct
QQYHSFPT

SEQ ID NO: 325 - protein, 9 aa, synthetic construct
QQYELYSYT

SEQ ID NO: 326 - protein, 8 aa, synthetic construct
QQYDTFIT

SEQ ID NO: 327 - protein, 8 aa, synthetic construct
EQYDSYPT

SEQ ID NO: 328 - protein, 17 aa, synthetic construct
RGRKASGSFYYYYGMDV

SEQ ID NO: 329 - protein, 7 aa, synthetic construct
GGSFSGY

SEQ ID NO: 330 - skipped

SEQ ID NO: 331 - protein, 7 aa, synthetic construct
RGPWSFD

SEQ ID NO: 332 - protein, 7 aa, synthetic construct
SQSISSW

SEQ ID NO: 333 - skipped

SEQ ID NO: 334 - protein, 6 aa, synthetic construct
YNSYPI

SEQ ID NO: 335 - protein, 8 aa, synthetic construct
SQSVSSSY

SEQ ID NO: 336 - skipped

SEQ ID NO: 337 - protein, 6 aa, synthetic construct
YGSSPI

SEQ ID NO: 338 - protein, 7 aa, synthetic construct
SQSIGSW

SEQ ID NO: 339 - protein, 5 aa, synthetic construct
YHSFY

SEQ ID NO: 340 - protein, 5 aa, synthetic construct
SNSYY

SEQ ID NO: 341 - protein, 5 aa, synthetic construct
YNSYP

SEQ ID NO: 342 - protein, 7 aa, synthetic construct
SQSISSY

SEQ ID NO: 343 - skipped

SEQ ID NO: 344 - protein, 6 aa, synthetic construct
SYDIPY

SEQ ID NO: 345 - protein, 6 aa, synthetic construct
YGSFPI

SEQ ID NO: 346 - protein, 6 aa, synthetic construct
SKEVPW

SEQ ID NO: 347 - protein, 5 aa, synthetic construct
YNSFP

SEQ ID NO: 348 - protein, 5 aa, synthetic construct
YDIYP

SEQ ID NO: 349 - protein, 5 aa, synthetic construct
YDSYP

SEQ ID NO: 350 - protein, 5 aa, synthetic construct
YGSFP

SEQ ID NO: 351 - protein, 5 aa, synthetic construct
YQSFP

SEQ ID NO: 352 - protein, 5 aa, synthetic construct
YSSFS

SEQ ID NO: 353 - protein, 5 aa, synthetic construct
YESYS

SEQ ID NO: 354 - protein, 5 aa, synthetic construct
YDSFI

SEQ ID NO: 355 - protein, 5 aa, synthetic construct
YQSYP

SEQ ID NO: 356 - protein, 5 aa, synthetic construct
YHSFP

SEQ ID NO: 357 - protein, 6 aa, synthetic construct
YELYSY

SEQ ID NO: 358 - protein, 5 aa, synthetic construct
YDTFI

SEQ ID NO: 359 - protein, 7 aa, synthetic construct
GGTFSSY

SEQ ID NO: 360 - protein, 4 aa, synthetic construct
PIFG

SEQ ID NO: 361 - protein, 14 aa, synthetic construct
DSSIRHAYYYYGMD

SEQ ID NO: 362 - protein, 13 aa, synthetic construct
SQSVLYSSNNKNY

SEQ ID NO: 363 - protein, 6 aa, synthetic construct
YYSTPI

SEQ ID NO: 364 - protein, 9 aa, synthetic construct
GGSISSSSY

SEQ ID NO: 365 - skipped

SEQ ID NO: 366 - protein, 9 aa, synthetic construct
SDRFHPYFD

SEQ ID NO: 367 - protein, 7 aa, synthetic construct
SQSVSRY

SEQ ID NO: 368 - skipped

SEQ ID NO: 369 - protein, 6 aa, synthetic construct
FDTWPP

SEQ ID NO: 370 - protein, 15 aa, synthetic construct
GRKASGSFYYYYGMD

SEQ ID NO: 371 - protein, 13 aa, synthetic construct
SQSLLNSGNQKNY

SEQ ID NO: 372 - protein, 6 aa, synthetic construct
DYSYPY

SEQ ID NO: 373 - protein, 4 aa, synthetic construct
PSGG

SEQ ID NO: 374 - protein, 15 aa, synthetic construct
APNYGDTTHDYYYMD

SEQ ID NO: 375 - protein, 7 aa, synthetic construct
SQSVSSN

SEQ ID NO: 376 - protein, 6 aa, synthetic construct
YDDWPF

SEQ ID NO: 377 - protein, 7 aa, synthetic construct
GYTFTGY

SEQ ID NO: 378 - protein, 4 aa, synthetic construct
PNSG

SEQ ID NO: 379 - protein, 13 aa, synthetic construct
TGEYYDTDDHGMD

SEQ ID NO: 380 - protein, 6 aa, synthetic construct
DDYWPP

SEQ ID NO: 381 - protein, 7 aa, synthetic construct
GFTFSSY

SEQ ID NO: 382 - protein, 4 aa, synthetic construct
GSGG

SEQ ID NO: 383 - protein, 10 aa, synthetic construct
GGYYDSGAGD

SEQ ID NO: 384 - protein, 7 aa, synthetic construct
SQGIDSW

SEQ ID NO: 385 - skipped

SEQ ID NO: 386 - protein, 6 aa, synthetic construct
GVSYPR

SEQ ID NO: 387 - protein, 14 aa, synthetic construct
GAGFAYGMDYYYMD

SEQ ID NO: 388 - protein, 7 aa, synthetic construct
SQSVSSY

SEQ ID NO: 389 - protein, 6 aa, synthetic construct
SDNWPF

SEQ ID NO: 390 - protein, 4 aa, synthetic construct
SSSS

SEQ ID NO: 391 - protein, 11 aa, synthetic construct
  VARIANT 3: note = M, L, I, V, Q, or F
APXGAAAGWFD

SEQ ID NO: 392 - protein, 7 aa, synthetic construct
SQGISSW

SEQ ID NO: 393 - protein, 6 aa, synthetic construct
GVSFPR

SEQ ID NO: 394 - protein, 11 aa, synthetic construct
APMGAAAGWFD

SEQ ID NO: 395 - protein, 11 aa, synthetic construct
APIGAAAGWFD

SEQ ID NO: 396 - protein, 11 aa, synthetic construct
APQGAAAGWFD

SEQ ID NO: 397 - protein, 11 aa, synthetic construct
APLGAAAGWFD

SEQ ID NO: 398 - protein, 11 aa, synthetic construct
APFGAAAGWFD

SEQ ID NO: 399 - protein, 11 aa, synthetic construct
APVGAAAGWFD

SEQ ID NO: 400 - protein, 8 aa, synthetic construct
GGSFSGYY

SEQ ID NO: 401 - protein, 7 aa, synthetic construct
IDHSGST

SEQ ID NO: 402 - protein, 6 aa, synthetic construct
QSISSW

SEQ ID NO: 403 - protein, 6 aa, synthetic construct
QSIGSW

SEQ ID NO: 404 - protein, 11 aa, synthetic construct
ARARGPWGFDP

SEQ ID NO: 405 - protein, 6 aa, synthetic construct
QSISSY

SEQ ID NO: 406 - protein, 8 aa, synthetic construct
GGTFSSYA

SEQ ID NO: 407 - protein, 8 aa, synthetic construct
IIPIFGTA

SEQ ID NO: 408 - protein, 12 aa, synthetic construct
QSVLYSSNNKNY

SEQ ID NO: 409 - protein, 10 aa, synthetic construct
GGSISSSSYY

SEQ ID NO: 410 - protein, 7 aa, synthetic construct
IYYSGST

SEQ ID NO: 411 - protein, 6 aa, synthetic construct
QSVSRY

SEQ ID NO: 412 - protein, 12 aa, synthetic construct
QSLLNSGNQKNY

SEQ ID NO: 413 - protein, 8 aa, synthetic construct
GYTFTSYY

SEQ ID NO: 414 - protein, 8 aa, synthetic construct
INPSGGST

SEQ ID NO: 415 - protein, 6 aa, synthetic construct
QSVSSN

SEQ ID NO: 416 - protein, 8 aa, synthetic construct
GYTFTGYY

SEQ ID NO: 417 - protein, 8 aa, synthetic construct
INPNSGGT

SEQ ID NO: 418 - protein, 8 aa, synthetic construct
GFTFSSYA

SEQ ID NO: 419 - protein, 8 aa, synthetic construct
ISGSGGST

SEQ ID NO: 420 - protein, 6 aa, synthetic construct
QGIDSW

SEQ ID NO: 421 - protein, 6 aa, synthetic construct
QSVSSY

SEQ ID NO: 422 - protein, 8 aa, synthetic construct
GFTFSSYS

SEQ ID NO: 423 - protein, 8 aa, synthetic construct
ISSSSSYI

SEQ ID NO: 424 - protein, 6 aa, synthetic construct
QGISSW

SEQ ID NO: 425 - protein, 10 aa, synthetic construct
VYGGSFSGYY

SEQ ID NO: 426 - protein, 16 aa, synthetic construct
IDHSGSTNYNPSLKSR

SEQ ID NO: 427 - protein, 8 aa, synthetic construct
ARGPWSFD

SEQ ID NO: 428 - protein, 8 aa, synthetic construct
ASQSISSW

SEQ ID NO: 429 - protein, 12 aa, synthetic construct
KASSLESGVPSR

SEQ ID NO: 430 - protein, 9 aa, synthetic construct
ASQSVSSSY

SEQ ID NO: 431 - protein, 12 aa, synthetic construct
GASSRATGIPDR

SEQ ID NO: 432 - protein, 8 aa, synthetic construct
ASQSIGSW

SEQ ID NO: 433 - protein, 8 aa, synthetic construct
ARGPWGFD

SEQ ID NO: 434 - protein, 8 aa, synthetic construct
TSQSISSY

SEQ ID NO: 435 - protein, 12 aa, synthetic construct
WASTRESGVPDR

SEQ ID NO: 436 - protein, 10 aa, synthetic construct
ASGGTFSSYA

SEQ ID NO: 437 - protein, 17 aa, synthetic construct
IIPIFGTANYAQKFQGR

SEQ ID NO: 438 - protein, 15 aa, synthetic construct
GDSSIRHAYYYYGMD

SEQ ID NO: 439 - protein, 14 aa, synthetic construct
SSQSVLYSSNNKNY

SEQ ID NO: 440 - protein, 12 aa, synthetic construct
VSGGSISSSSYY

SEQ ID NO: 441 - protein, 16 aa, synthetic construct
IYYSGSTYYNPSLKSR

SEQ ID NO: 442 - protein, 10 aa, synthetic construct
GSDRFHPYFD

SEQ ID NO: 443 - protein, 8 aa, synthetic construct
ASQSVSRY

SEQ ID NO: 444 - protein, 12 aa, synthetic construct
DASNRATGIPAR

SEQ ID NO: 445 - protein, 16 aa, synthetic construct
RGRKASGSFYYYYGMD

SEQ ID NO: 446 - protein, 14 aa, synthetic construct
SSQSLLNSGNQKNY

SEQ ID NO: 447 - protein, 10 aa, synthetic construct
ASGYTFTSYY

SEQ ID NO: 448 - protein, 17 aa, synthetic construct
INPSGGSTSYAQKFQGR

SEQ ID NO: 449 - protein, 16 aa, synthetic construct
GAPNYGDTTHDYYYMD

SEQ ID NO: 450 - protein, 8 aa, synthetic construct
ASQSVSSN

SEQ ID NO: 451 - protein, 12 aa, synthetic construct
GASTRATGIPAR

SEQ ID NO: 452 - protein, 10 aa, synthetic construct
ASGYTFTGYY

SEQ ID NO: 453 - protein, 17 aa, synthetic construct
INPNSGGTNYAQKFQGR

SEQ ID NO: 454 - protein, 14 aa, synthetic construct
DTGEYYDTDDHGMD

SEQ ID NO: 455 - protein, 10 aa, synthetic construct
ASGFTFSSYA

SEQ ID NO: 456 - protein, 17 aa, synthetic construct
ISGSGGSTYYADSVKGR

SEQ ID NO: 457 - protein, 11 aa, synthetic construct
DGGYYDSGAGD

SEQ ID NO: 458 - protein, 8 aa, synthetic construct
ASQGIDSW

SEQ ID NO: 459 - protein, 12 aa, synthetic construct
AASSLQSGVPSR

SEQ ID NO: 460 - protein, 15 aa, synthetic construct
EGAGFAYGMDYYYMD

SEQ ID NO: 461 - protein, 8 aa, synthetic construct
ASQSVSSY

SEQ ID NO: 462 - protein, 10 aa, synthetic construct
ASGFTFSSYS

SEQ ID NO: 463 - protein, 17 aa, synthetic construct
ISSSSSYIYYADSVKGR

SEQ ID NO: 464 - protein, 12 aa, synthetic construct
  VARIANT 4: note = M, L, I, V, Q, or F
GAPXGAAAGWFD

SEQ ID NO: 465 - protein, 8 aa, synthetic construct
ASQGISSW

SEQ ID NO: 466 - protein, 12 aa, synthetic construct
GAPMGAAAGWFD

SEQ ID NO: 467 - protein, 12 aa, synthetic construct
GAPIGAAAGWFD

SEQ ID NO: 468 - protein, 12 aa, synthetic construct
GAPQGAAAGWFD

SEQ ID NO: 469 - protein, 12 aa, synthetic construct
GAPLGAAAGWFD

SEQ ID NO: 470 - protein, 12 aa, synthetic construct
GAPFGAAAGWFD

SEQ ID NO: 471 - protein, 12 aa, synthetic construct
GAPVGAAAGWFD

SEQ ID NO: 472 - protein, 5 aa, synthetic construct
DFGMH

SEQ ID NO: 473 - protein, 17 aa, synthetic construct
YISSGSSTIYYADTVKG

SEQ ID NO: 474 - protein, 17 aa, synthetic construct
YISSGSSTIYYADSVKG

SEQ ID NO: 475 - protein, 17 aa, synthetic construct
  VARIANT 14: note = S or T
YISSGSSTIYYADXVKG

SEQ ID NO: 476 - protein, 5 aa, synthetic construct
DYYMD

SEQ ID NO: 477 - protein, 17 aa, synthetic construct
YIFPNDASTTYNEKFKG

SEQ ID NO: 478 - protein, 5 aa, synthetic construct
DYGMH

SEQ ID NO: 479 - protein, 17 aa, synthetic construct
YISSGSSTFYYADTVKG

SEQ ID NO: 480 - protein, 5 aa, synthetic construct
SYWMH

SEQ ID NO: 481 - protein, 17 aa, synthetic construct
KIDSSDSKTQYNQKFKD

SEQ ID NO: 482 - protein, 4 aa, synthetic construct
SGSS

SEQ ID NO: 483 - protein, 9 aa, synthetic construct
QSYYRGTLD

SEQ ID NO: 484 - protein, 11 aa, synthetic construct
SQSVTTSNYNY

SEQ ID NO: 485 - skipped

SEQ ID NO: 486 - protein, 6 aa, synthetic construct
SWEIPW

SEQ ID NO: 487 - protein, 4 aa, synthetic construct
PNDA

SEQ ID NO: 488 - protein, 4 aa, synthetic construct
RDAD

SEQ ID NO: 489 - protein, 6 aa, synthetic construct
KSSVSY

SEQ ID NO: 490 - skipped

SEQ ID NO: 491 - protein, 6 aa, synthetic construct
WSSYPY

SEQ ID NO: 492 - protein, 9 aa, synthetic construct
QSYYRGTMD

SEQ ID NO: 493 - protein, 11 aa, synthetic construct
SQGVSTSTYTY

SEQ ID NO: 494 - protein, 9 aa, synthetic construct
QSYYRGTID

SEQ ID NO: 495 - protein, 4 aa, synthetic construct
SSDS

SEQ ID NO: 496 - protein, 6 aa, synthetic construct
GYLWFA

SEQ ID NO: 497 - protein, 11 aa, synthetic construct
SQSVSTSTYSY

SEQ ID NO: 498 - skipped

SEQ ID NO: 499 - protein, 8 aa, synthetic construct
RFTFSDFG

SEQ ID NO: 500 - protein, 8 aa, synthetic construct
ISSGSSTI

SEQ ID NO: 501 - protein, 13 aa, synthetic construct
ASSQSYYRGTLDY

SEQ ID NO: 502 - protein, 10 aa, synthetic construct
QSVTTSNYNY

SEQ ID NO: 503 - protein, 8 aa, synthetic construct
GYKFTDYY

SEQ ID NO: 504 - protein, 8 aa, synthetic construct
IFPNDAST

SEQ ID NO: 505 - protein, 8 aa, synthetic construct
ARSRDADY

SEQ ID NO: 506 - protein, 5 aa, synthetic construct
SSVSY

SEQ ID NO: 507 - protein, 8 aa, synthetic construct
GFTFSDYG

SEQ ID NO: 508 - protein, 8 aa, synthetic construct
ISSGSSTF

SEQ ID NO: 509 - protein, 13 aa, synthetic construct
GSSQSYYRGTMDY

SEQ ID NO: 510 - protein, 10 aa, synthetic construct
QGVSTSTYTY

SEQ ID NO: 511 - protein, 13 aa, synthetic construct
GSSQSYYRGTIDY

SEQ ID NO: 512 - protein, 8 aa, synthetic construct
GYTFTSYW

SEQ ID NO: 513 - protein, 8 aa, synthetic construct
IDSSDSKT

SEQ ID NO: 514 - protein, 10 aa, synthetic construct
ARGGYLWFAY

SEQ ID NO: 515 - protein, 10 aa, synthetic construct
QSVSTSTYSY

SEQ ID NO: 516 - protein, 10 aa, synthetic construct
ASRFTFSDFG

SEQ ID NO: 517 - protein, 17 aa, synthetic construct
ISSGSSTIYYADTVKGR

SEQ ID NO: 518 - protein, 10 aa, synthetic construct
SQSYYRGTLD

SEQ ID NO: 519 - protein, 12 aa, synthetic construct
ASQSVTTSNYNY

SEQ ID NO: 520 - protein, 12 aa, synthetic construct
FASNLESGVPAR

SEQ ID NO: 521 - protein, 17 aa, synthetic construct
ISSGSSTIYYADSVKGR

SEQ ID NO: 522 - protein, 12 aa, synthetic construct
FASNLESGVPSR

SEQ ID NO: 523 - protein, 17 aa, synthetic construct
  VARIANT 13: note = S or T
ISSGSSTIYYADXVKGR

SEQ ID NO: 524 - protein, 10 aa, synthetic construct
ASGYKFTDYY

SEQ ID NO: 525 - protein, 17 aa, synthetic construct
IFPNDASTTYNEKFKGK

SEQ ID NO: 526 - protein, 5 aa, synthetic construct
SRDAD

SEQ ID NO: 527 - protein, 7 aa, synthetic construct
AKSSVSY

SEQ ID NO: 528 - protein, 12 aa, synthetic construct
DTSYLGSGIPAR

SEQ ID NO: 529 - protein, 10 aa, synthetic construct
ASGFTFSDYG

SEQ ID NO: 530 - protein, 17 aa, synthetic construct
ISSGSSTFYYADTVKGR

SEQ ID NO: 531 - protein, 10 aa, synthetic construct
SQSYYRGTMD

SEQ ID NO: 532 - protein, 12 aa, synthetic construct
ASQGVSTSTYTY

SEQ ID NO: 533 - protein, 10 aa, synthetic construct
SQSYYRGTID

SEQ ID NO: 534 - protein, 10 aa, synthetic construct
ASGYTFTSYW

SEQ ID NO: 535 - protein, 17 aa, synthetic construct
IDSSDSKTQYNQKFKDK

SEQ ID NO: 536 - protein, 7 aa, synthetic construct
GGYLWFA

SEQ ID NO: 537 - protein, 12 aa, synthetic construct
ASQSVSTSTYSY

SEQ ID NO: 538 - protein, 12 aa, synthetic construct
YASNLESGVPAR

SEQ ID NO: 539 - protein, 17 aa, synthetic construct
IDSSDSKTQYNQKFKDR

SEQ ID NO: 540 - protein, 12 aa, synthetic construct
YASNLESGVPDR

SEQ ID NO: 541 - protein, 15 aa, synthetic construct
  VARIANT 14: note = L, M, or V
RASQSVTTSNYNYXH

SEQ ID NO: 542 - protein, 12 aa, synthetic construct
  VARIANT 11: note = A or S
FASNLESGVPXR

SEQ ID NO: 543 - protein, 15 aa, synthetic construct
  SITE 1: note = Acetylated arginine
  SITE 15: note = Arginine amide
RRCPLYISYDPVCRR

SEQ ID NO: 544 - protein, 19 aa, synthetic construct
  SITE 1: note = Acetylated arginine
  SITE 19: note = Arginine amide
RRRRCPLYISYDPVCRRRR

SEQ ID NO: 545 - DNA, 1437 bp, synthetic construct
  source 1..1437: mol_type = other DNA
gacatccagctgacacagagccctagcagcctgtctgcctctgtgggcgacagagtgaccatcacctgtagagccagccagagcgtgaccaccagcaactacaactacatgcactggttccagcagaagcccggcaaggcccctaagctgctgatcaagttcgccagcaacctggaaagcggcgtgcccagcagattttctggcagcggctctggcaccgacttcaccctgaccatatctagcctgcagcctgaggacttcgccacctactactgtcagcacagctgggagatcccttggacctttggctgtggcaccaaggtggaaatcaaaggcggcggaggatctggcggaggtggaagcggaggcggaggaagcggtggcggcggatctgaagttcagctggttgaaagtggcggaggcctggttaagcctggcggatctctgagactgagctgtgccgcctccagattcaccttcagcgacttcggaatgcactgggtccgacaggcccctggcaaatgtctggaatgggtgtcctacatcagcagcggcagcagcaccatctactacgccgatagcgtgaagggcagattcacaatcagccgggacaacgccaagaacagcctgtacctgcagatgaactccctgagagccgaggacaccgccgtgtactattgtgccagcagccagtcctactaccggggcacactggattattggggccagggcacaaccgtgacagtgtccagcgccagcgataagacccacacctgtcctccatgtcctgctccagaactgctcggcggaccttccgtgttcctgtttcctccaaagcctaaggacaccctgatgatctccagaacacccgaagtgacctgcgtggtggtggatgtgtctcacgaggaccctgaagtgaagttcaattggtacgtggacggcgtggaagtgcacaatgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctcgcgagcctagagtgtataccttgcctccatgccgggacgagctgaccaagaatcaggtgtccctgacctgcctggtcaagggcttctacccttccgatatcgccgtggaatgggagagcaatggccagcctgagaacaactacaagaccacacctcctgtgctggtgtccgacggcagctttaccctgtacagcaagctgacagtggacaagagcagatggcagcagggcaacgtgttcagctgcagcgtgatgcacgaggccctgcacaaccactacacccagaagtccctgagcctgtctcctgga

SEQ ID NO: 546 - DNA, 1353 bp, synthetic construct
  source 1..1353: mol_type = other DNA
gaggtgcagctggttgaatctggcggcggacttgtgaagcctggcggatctctgagactgagctgtgccgccagcggcttcacctttagcagctacagcatgaactgggtccgacaggcccctggcaaaggccttgaatgggtgtccagcatcagcagcagctccagctacatctactacgccgacagcgtgaagggcagattcaccatcagccgggacaacgccaagaacagcctgtacctgcagatgaactccctgagagccgaggacaccgccgtgtactattgtgctagaggcgcccctattggagccgccgctggatggttcgatccttggggacagggaaccctggtcaccgtttcttctgccagcacaaagggccctagcgttttcccactggctcccagcagcaagagcacaagcggaggaacagctgccctgggctgtctggtcaaggactactttcctgagcctgtgaccgtgtcctggaacagcggagcactgactagcggcgtgcacacatttccagccgtgctgcaaagcagcggcctgtactctctgagcagcgtcgtgacagtgcctagcagctctctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcctgctcctatcgagaaaaccatcagcaaggccaagggccagcctcgcgaacctcaagtctgtacactgcctcctagccgggatgagctgaccgagaatcaggtgtccctgacctgtctcgtgaagggcttctacccctccgatatcgccgtggaatgggagagcaatggccagccagagaacaactacaagacaacccctcctgtgctggacagcgacggctcattcttcctgtacagctggctgaccgtggacaagtccagatggcagcagggcaacgtgttctcctgcagcgtgatgcacgaggccctgcacaaccactacacccagaagtccctgtctctgagcccaggc

SEQ ID NO: 547 - DNA, 642 bp, synthetic construct
  source 1..642: mol_type = other DNA
gacatccagatgacacagagccccagctccgtgtctgcctctgtgggagacagagtgaccatcacctgtagagccagccagggcatctcttcttggctggcctggtatcagcagaagcctggcaaggcccctaagctgctgatctatgccgctagctctctgcagtctggcgtgccctctagattttctggcagcggctctggcaccgacttcaccctgaccatatctagcctgcagcctgaggacttcgccacctactattgtcagcagggcgtgtcctttccacggacctttggcggcggaacaaaggtggaaatcaagcggacagtggccgctcctagcgtgttcatctttccacctagcgacgagcagctgaagtccggcacagcctctgttgtgtgcctgctgaacaacttctaccccagagaagccaaggtgcagtggaaggtggacaatgccctgcagagcggcaacagccaagagagcgtgacagagcaggacagcaaggactccacctacagcctgagcagcaccctgacactgagcaaggccgactacgagaagcacaaagtgtacgcctgcgaagtgacccaccagggcctttctagccctgtgaccaagagcttcaaccggggcgagtgt

SEQ ID NO: 548 - DNA, 1437 bp, synthetic construct
  source 1..1437: mol_type = other DNA
gacatccaactgacccagtccccgtcctccctgagcgcatctgtcggcgaccgggtgaccatcacctgccgggcctcgcagtccgtgaccacgtcgaactacaactacatgcactggttccagcagaagcccggtaaggcgcccaagctgctcatcaagttcgcgtccaacctggagtcgggcgtgccgtcgcggttctccggctccggcagcggcaccgacttcacgctgacgatctcgtccctccagcccgaggacttcgccacgtactactgccagcactcctgggagatcccgtggacgttcgggtgcggcaccaaggtcgagatcaagggaggcgggggcagtgggggcggaggctctgggggcggcggcagtggcggcggcggctcggaggtccagctggtagagtccggaggcggcctcgtgaagccgggcggctccctgcgcctgtcctgcgcggccagccgtttcaccttctcggacttcgggatgcattgggtgcgccaggccccaggcaagtgcctggagtgggtttcctacatctcgtccggctcgagtaccatctattacgccgacagcgtcaagggacgcttcacgatctcgcgggacaacgccaagaacagcctttacctgcagatgaacagcctgcgcgcggaggacaccgccgtctactactgcgcctcgtcccagagctattaccggggtaccctcgactactggggccaggggacgacggtgaccgtcagctccgccagcgacaagacccacacgtgccccccgtgcccggccccggagctgctgggaggcccgtccgtgttcctgttccccccgaagcccaaggacaccctgatgatctcgcgcaccccggaggtgacctgcgtagtcgtcgacgtgtcccacgaggaccccgaggtgaagttcaactggtacgtggacggcgtcgaggtccacaacgcgaagaccaagccgcgcgaggagcagtacaacagcacctaccgggtcgtgagcgtcctgaccgtgctgcaccaagactggctcaacggtaaggagtacaagtgtaaggtgagcaacaaggccctccctgccccgattgagaagaccatctcgaaggctaagggccagccgcgggagccccgggtgtacacgctcccgccctgccgggacgaactgaccaaaaaccaggtgtccctgacgtgtctggtgaagggcttctacccctccgacatcgccgtcgagtgggagtccaatggtcagcccgagaacaactacaagacgaccccgcccgtgctggtgagcgacgggtccttcaccctgtattccaagctcacggtcgacaagtcccgctggcagcagggcaacgtcttctcgtgctccgtgatgcacgaggccctccacaaccactacacccagaagtcactgtcactctcccccggc

SEQ ID NO: 549 - DNA, 1353 bp, synthetic construct
  source 1..1353: mol_type = other DNA
gaggtgcagctggttgaatctggcggcggacttgtgaagcctggcggatctctgagactgtcttgtgccgcctccggcttcaccttctccagctactctatgaactgggtccgacaggcccctggcaaaggactggaatgggtgtcctctatctcctccagctcctcctacatctactacgccgactccgtgaagggcagattcaccatcagccgggacaacgccaagaactccctgtacctgcagatgaacagcctgagagccgaggacaccgccgtgtactattgtgctagaggcgctcctatcggcgctgccgctggatggttcgatccttggggacagggaaccctggtcaccgtgtcctctgcttctaccaagggacccagcgtgttccctctggctccttccagcaagtctacctctggcggaacagctgctctgggctgcctggtcaaggactactttcctgagcctgtgaccgtgtcttggaactctggcgctctgacatccggcgtgcacacatttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaatgccaagaccaagcctagagaggaacagtacaactccacctacagagtggtgtccgtgctgaccgtgctgcaccaggattggctgaacggcaaagagtacaagtgcaaggtgtccaacaaggccctgcctgctccaatcgaaaagaccatctccaaggccaagggccagcctcgggaacctcaagtctgtaccttgcctcctagccgggatgagctgaccgagaatcaggtgtccctgacctgcctcgtgaagggattctacccctccgatatcgccgtggaatgggagtctaatggccagccagagaacaactacaagacaacccctcctgtgctggactccgacggctccttctttctgtactcctggctgacagtggacaagtccagatggcagcagggcaacgtgttctcctgctccgtgatgcacgaggccctgcacaatcactacacccagaagtccctgtctctgtcccctggc

SEQ ID NO: 550 - DNA, 642 bp, synthetic construct
  source 1..642: mol_type = other DNA
gacatccagatgacccagtctccatcctccgtgtctgcctctgtgggcgacagagtgaccatcacctgtagagccagccagggcatctcttcttggctggcctggtatcagcagaagcctggcaaggctcccaagctgctgatctacgctgcttccagtctgcagtctggcgtgccctccagattttccggctctggctctggcaccgactttaccctgacaatctccagcctgcagcctgaggacttcgccacctactattgtcagcagggcgtgtcctttcctcggacctttggcggaggcaccaaggtggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgctctgcagtccggcaactcccaagagtccgtgaccgagcaggactccaaggactctacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 551 - protein, 7 aa, synthetic construct
RGPWGFD

SEQ ID NO: 552 - protein, 7 aa, synthetic construct
QSVSSSY